(19) United States
(12) Patent Application Publication
(10) Pub. No.: US 2025/0186606 A1
(43) Pub. Date: Jun. 12, 2025

(54) MODULAR LINKER COMPOUND FOR TARGET-BINDING DRUG CONJUGATES

(71) Applicant: Heidelberg Pharma Research GmbH, Ladenburg (DE)

(72) Inventors: Christoph Mueller, Ladenburg (DE); Hendrik Gruss, Ladenburg (DE); Torsten Hechler, Ladenburg (DE); Valentin Petrich, Ladenburg (DE)

(21) Appl. No.: 18/972,131

(22) Filed: Dec. 6, 2024

(30) Foreign Application Priority Data
Dec. 6, 2023 (EP) 23214774.4

(51) Int. Cl. A61K 47/68; A61K 47/54; A61K 47/65; A61K 47/69

(52) U.S. Cl. CPC A61K 47/6889; A61K 47/545; A61K 47/65; A61K 47/68031; A61K 47/68037; A61K 47/6809; A61K 47/6849; A61K 47/6871; A61K 47/6951

(57) ABSTRACT

The present disclosure relates to modular linker compounds for target-binding drug conjugates (TBDCs) and in particular to modular linker compounds for antibody-drug-conjugates (ADCs). The present disclosure further relates to methods of synthesizing the modular linker compound, to target-binding drug conjugates comprising the modular linker compound of the present disclosure as well as to pharmaceutical compositions comprising target-binding drug conjugates compound of the present disclosure. Embodiments of the present disclosure have been particularly developed as target-binding drug conjugates for use in the treatment of cancer and will be described hereinafter with reference to this application. However, it will be appreciated that the present disclosure is not limited to this particular field of use.

### CROSS REFERENCE TO RELATED APPLICATIONS

This application is a nonprovisional application which claims priority to European Patent Application No. 23214774.4, filed on 6 Dec. 2023, the entire contents of which are incorporated herein by reference.

### BACKGROUND

### Field

The present disclosure relates to modular linker compounds for target-binding drug conjugates (TBDCs) and in particular to modular linker compounds for antibody-drug conjugates (ADCs). The present disclosure further relates to methods of synthesizing the modular linker compound, to target-binding drug conjugates comprising the modular linker compound of the present disclosure as well as to pharmaceutical compositions comprising target-binding drug conjugates compound of the present disclosure. Embodiments of the present disclosure have been particularly developed as target-binding drug conjugates for use in the treatment of cancer and will be described hereinafter with reference to this application. However, it will be appreciated that the present disclosure is not limited to this particular field of use.

Aside from surgical resection and radiation therapy, chemotherapy remains the therapeutic option predominantly utilized in the treatment of cancer. However, it is also well-established that chemotherapy-through its limited selectivity and specificity towards cancer cells-typically offers only a small therapeutic window for the administration of highly toxic doses of chemotherapeutic agents, which regularly lead to severe side effects for the patients through the toxic effect on non-cancer cells, while still only conveying a low efficacy towards malign cancer cells.

Targeted drug therapy solutions have been developed to increase the specific delivery of cytotoxic drug substances directly to malign cancer cells. In particular, target-binding drug conjugates based on the principles of target sequence or epitope recognition and binding known from immunoglobulins/antibodies provide new treatment options that allow for the delivery of drug substances to malign cancer cells with high specificity and with very limited or no effect on non-cancer cells.

Target-binding drug conjugates (TBDCs), and in particular antibody-drug conjugates (ADCs), typically consist of a target-binding moiety covalently linked to cytotoxic drug substances via a synthetic linker. While (a) the target-binding moiety ensures the high degree of specificity and (b) the cytotoxic drug substance ensures the effect on the cancer cells once delivered, it is (c) the linker compound that substantially affects the therapeutic index, efficacy and pharmacokinetics of such conjugates.

In particular, the linker compound must (i) ensure high plasma stability of the conjugate in circulation to prevent premature drug release and consequential undesired systemic off-target effects; (ii) maintain the properties of the target-binding moiety as well as of the cytotoxic drug substance, (iii) provide a high degree of aqueous solubility of the conjugate to allow for the delivery of hydrophobic/lipophilic drug substances and to prevent aggregation of the TBDCs in a pharmaceutical composition; and (iv) ensure appropriate exposure of the targeted cancer cells to the cytotoxic drug substance to maximize the therapeutic effect.

The lipophilic nature of many cytotoxic drug substances can adversely affect the properties of a TBDC to the extent that the payloads are not efficiently delivered to the target cells. Modulating TBDC properties by affixing polar groups in linkers may reduce aggregation during conjugation and improve physiochemical properties of the ADCs leading to improved pharmacokinetics (PK) and an improved therapeutic index of the ADC (see, for example, Nature Biotechnology, 2015, 33, 733-735). Modulation of the linker to effect a change in the polarity or charge of the final metabolite may improve activity toward multidrug resistant (MDR) cells owing to better retention of the payloads inside the cells (see, for example, Cancer Res; 70 (6) Mar. 15, 2010, p2528). The chemical moieties on linkers, e.g., polyethylene glycol (PEG), sugar, or sulfuric acid groups (See, for example, WO2014062697, US20100323973), not only affect the conjugation efficiency and the ease of production of ADCs, but also often remain as part of the metabolites and thus affect the ADC's activity and safety.

With respect to the delivery of the cytotoxic drug substance at the target site, non-cleavable as well as cleavable linkers have been developed. Non-cleavable linkers, e.g. comprising thioether or maleimido-caproyl linkages, typically require internalisation and subsequent lysosomal enzymatic degradation of the entire TBDC by the target cell to release the cytotoxic drug substance. Cleavable linkers can be divided into the groups of chemically cleavable linkers and enzymatically cleavable linkers. Chemically cleavable linkers typically rely on pH sensitivity (lysosomal/endosomal release of cytotoxic drug substance) or reduction sensitivity (cytoplasmic release of cytotoxic drug substance) while various enzymatically cleavable are known that rely on enzymatic release of the cytotoxic drug substance, typically within the lysosomal compartment of the target cell. A combination of a cleavable peptide structure and a self-immolative spacer between the targeting-binding antibody and the drug of an ADC, for example, has been described to be applicable to a payload carrying a primary or secondary amine. Jeffrey et al. 2005 (Design, Synthesis, and in Vitro Evaluation of Dipeptide-Based Antibody Minor Groove Binder Conjugates. J. Med. Chem. Vol. 48:1344-1358). Notwithstanding, in many instances, the cytotoxic drug substance released still contains linker-derived adducts. In some instances, clever design of the cleavable linkage allows for a positive modification of the drug substance. In others, efficacy of the drug substance is negatively impacted by residual linker adducts.

However, the therapeutic efficacy of a TBDC is not only dependent on the appropriate release of the cytotoxic drug substance but also on the effective bio-conjugation connecting the drug substance to the target-binding moiety and, further, on the number molecules of the cytotoxic drug substance successfully linked to a single target-binding moiety. Given that TBDCs have been developed as ADCs primarily so far, this is known as the Drug-Antibody-Ratio (DAR). A low DAR is known to decrease TBDC efficacy and requires a highly toxic drug substance to be conjugated, whereas a high DAR has been linked to potential TBDC instability leading to increased systemic effects, a reduced TBDC half-life and a general alteration of the pharmacokinetics of the molecule. However, given that stochastic chemistries have typically been applied to conjugate drug substances to antibodies, even FDA-approved ADCs are composed of a mixture of conjugates, which are heterogeneous with respect to their DAR.

In view of prior art there is a need in the art for improved and widely applicable linker compounds for target-binding drug conjugates (TBDCs), which mediate stable conjugation of the cytotoxic drug substance to the target-binding moiety, while allowing for easy adaptation of a TBDC's Drug-Antibody-Ratio (DAR) and/or aqueous solubility depending on the cytotoxicity and hydrophobicity of the drug substance to be conjugated, respectively.

It is an object of the present disclosure to overcome or ameliorate at least one of the disadvantages of the prior art, or to provide a useful alternative. In particular, it is an object of the present disclosure to provide improved linker compounds for target-binding drug conjugates (TBDCs), in particular for antibody-drug conjugates (ADCs).

### SUMMARY

As indicated above, the present disclosure aims to provide linker compounds for target-binding drug conjugates (TBDCs), which are easily adaptable with respect to their Drug-Antibody-Ratio (DAR) and/or aqueous solubility depending on the cytotoxicity and hydrophobicity of the drug substance to be conjugated, respectively, while ensuring a homogeneousDegree-of-Labelling (DOL).

The here-described linker compounds provide a high degree of adaptability due to their modular architecture. Specifically, the modular linker compound of the present disclosure comprises at least one functional unit of a cleavable peptide sequence connected to a self-immolative spacer, to which both a solubility enhancing group and the cytotoxic drug substance are bound in physical proximity. Further, a chemical spacer connects this functional unit to a multimeric core comprising one or more branching units, wherein the multimeric core is customisable with respect to the number of functional units the modular linker compound of the present disclosure can carry and may further even accommodate a further direct linkage to a second cytotoxic drug substance outside of the functional unit described directly above.

In the modular linker compound, one branching unit is linked to a terminal, reactive moiety via a further chemical spacer, wherein the reactive moiety is suitable for reliable conjugation of the modular linker compound to a selected target-binding moiety. Typically, this target-binding moiety relies on the immunoglobulin principle to recognize and bind a target sequence or epitope of an antigen with high specificity.

Accordingly, in a first aspect the present disclosure relates to a modular linker compound of Formula (I)

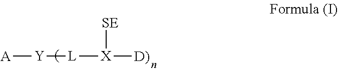

or a pharmaceutically acceptable salt or solvate thereof, wherein:
X is a self-immolative spacer

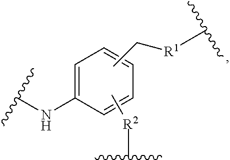

wherein:
R¹ covalently links X to D in para, meta or ortho to the amino group of X and is selected from a group consisting of a bond,

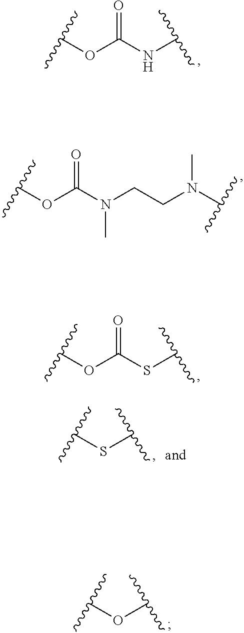

R² connects X to SE and is

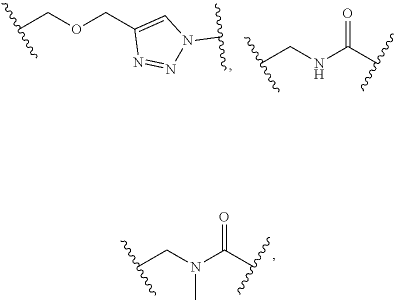

### or —O—;

D is a pharmaceutically active substance;
SE is a solubility enhancing group;
L is a spacer comprising a peptide covalently linked to X;
n is an integer of at least 2, optionally n is an integer from 2 to 5, e.g. 2, 3, 4 or 5;
Y is a branching unit,
A is a reactive moiety for conjugation to a target-binding moiety.

In some embodiments, L is

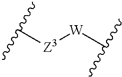

wherein:
W is a cleavable and non-cleavable peptide sequence connecting Z³ and X and is selected from the group consisting of:
Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gln, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gln, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and
Z³ is a bond or a spacer comprising a C₁₋₂₀ alkyl chain or a 1 to 10 polyethylene glycol (PEG)₁₋₁₀ or a C₂-amide-PEG₁₋₁₀,
wherein (PEG)₁₋₁₀ is

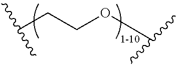

and C₂-amide-PEG₁₋₁₀ is

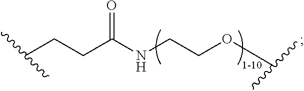

optionally L is

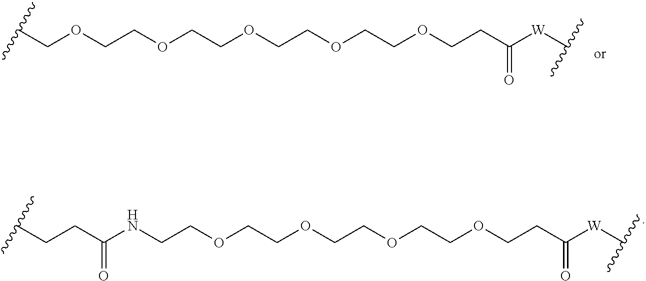

In a specific embodiment, if D, in the modular linker compound of Formula (Ia), has a ClogP of lower than −1

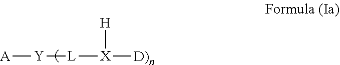

or a pharmaceutically acceptable salt or solvate thereof R² is H and no SE is attached to the modular linker compound.

(PEG)₁₋₁₀ and C₂-amide-PEG₁₋₁₀ as disclosed expressively include chain lengths of 1, 2, 3, 4, 5, 6, 7, 8, 9 and 10.

In a second aspect, the present disclosure relates to a method for synthesizing a modular linker compound of Formula (I) according to the first aspect, the method comprising:
(i) Contacting a first compound comprising:
   an immolative moiety,
   a cleavable peptide sequence W,
   an amine protecting group Pg¹, which is covalently linked to W,
   a pharmaceutically active substance D and
   a first complementary group Cg¹
with a second compound comprising
   a solubility enhancing group SE and
   a second complementary group Cg²
to form an intermediate of Formula II:

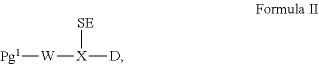

wherein SE is connected to X via R² and wherein R² is formed by the reaction of Cg¹ and Cg²;
(ii) Removing the amine protecting group Pg¹ from Formula II to expose a primary amine;
(iii) Contacting the primary amine with a third compound comprising
   a spacer Z³,
   an amine reactive group, and
   a third complementary group Cg³
to form the intermediate of Formula III:

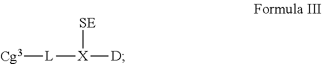

wherein L is

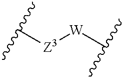

(iv) Contacting the intermediate of Formula III with a branched fourth compound comprising
   a fourth complementary group Cg⁴ and
   a fifth complementary group Cg⁵,
wherein Cg³ reacts with Cg⁴ to form Z²;
(v) Contacting the product of (iv) with a fifth compound comprising
   a reactive moiety A for conjugation to a target-binding moiety and
   a sixth complementary group Cg⁶
wherein reaction of Cg⁵ and Cg⁶ forms Z¹, to form the modular linker compound of Formula I:

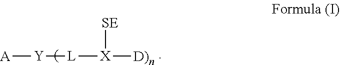

In a specific embodiment of the second aspect, if D has a ClogP of lower than −1, the present disclosure relates to a method for synthesizing a modular linker compound of Formula (Ia) according to the first aspect, the method comprising:
(i) Selecting a first compound comprising:
   an immolative moiety,
   a cleavable peptide sequence W,
   an amine protecting group Pg¹, which is covalently linked to W,
   a pharmaceutically active substance D
as an intermediate of Formula IIa:

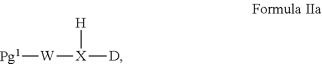

(ii) Removing the amine protecting group Pg¹ from Formula IIa to expose a primary amine;
(iii) Contacting the primary amine with a third compound comprising
   a spacer Z³,
   an amine reactive group, and
   a third complementary group Cg³
to form the intermediate of Formula IIIa:

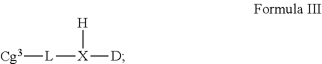

wherein L is

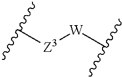

(iv) Contacting the intermediate of Formula IIIa with a branched fourth compound comprising
   a fourth complementary group Cg⁴ and
   a fifth complementary group Cg⁵,
wherein Cg³ reacts with Cg⁴ to form Z²;
(v) Contacting the product of (iv) with a fifth compound comprising
   a reactive moiety A for conjugation to a target-binding moiety and
   a sixth complementary group Cg⁶
wherein reaction of Cg⁵ and Cg⁶ forms Z¹, to form the modular linker compound of Formula Ia:

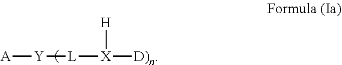

In a third aspect, the present disclosure relates to a target-binding drug conjugate (TBDC) comprising at least one reaction product of the modular linker compound according to the first aspect and a correspondingly reactive group of a target-binding moiety, wherein the at least one reaction product is the product of a reaction between A of the modular linker compound and the correspondingly reactive group of the target-binding moiety, which covalently couples the modular linker compound to the target-binding moiety. In some embodiments, the correspondingly reactive group of the target-binding moiety is a thiol group.

In some embodiments, the target-binding moiety of the target-binding drug conjugate of the third aspect specifically recognizes and binds a target sequence or epitope of a cancer-specific or cancer-associated antigen of Guanylate Cyclase C (GUCY2C), Cluster of Differentiation 33 (CD33), Cluster of Differentiation 30 (CD30), Cluster of Differentiation (CD5), Cluster of Differentiation 19 (CD19), Cluster of Differentiation 20 (CD20), Cluster of Differentiation 37 (CD37), Cluster of Differentiation 45 (CD45), Cluster of Differentiation 47 (CD47), Cluster of Differentiation 56 (CD56), Cluster of Differentiation 70 (CD70), Cluster of Differentiation 79b (CD79b), Cluster of Differentiation 117 (CD117/c-Kit), Cluster of Differentiation 123 (CD123/IL3RA), Cluster of Differentiation 137 (CD137/4-1BB), Cluster of Differentiation 138 (CD138), Cluster of Differentiation 269 (CD269/B-cell maturation antigen, BCMA), claudin 18.2, CEA (Carcinoembryonic antigen), Epithelial Cell Adhesion Molecule (EpCam), DKK1 (Dickkopf-related protein 1), glypican-3, muscin-1 (MUC-1/CD227), Mucin 5AC (MUC5AC), Carbonic Anhydrase 9 (CAIX), C—X—C Motif Chemokine Receptor 1 (CXCR1, CD181), C—X—C Motif Chemokine Receptor 2 (CXCR2/CD182), human epidermal growth factor receptor 2 (HER2), Cluster of Differentiation 22 (CD22), Nectin Cell Adhesion Molecule 4 (Nectin 4), Tumor-associated calcium signal transducer 2 (Trop-2), Delta-like 3 (DLL3), Transmembrane glycoprotein NMB (gpNMB), prostate-specific membrane antigen (PSMA), Hepatocyte growth factor receptor (c-Met, CD151), Solute Carrier Family 39 Member 6 (LIV-1), Folate receptor 1 (FOLR1), Mesothelin, Carbonic anhydrase 6 (CA6), epidermal growth factor receptor (EGFR), or Ectonucleotide pyrophosphatase/phosphodiesterase family member 3 (ENPP3), Mucin 17 (MUC17), claudin 23 (CLDN23).

In some embodiments, the target-binding moiety of the target-binding drug conjugate of the third aspect is any one of Adecatumumab (anti-EpCam), Amatuximab, (anti-mesothelin), Amivantamab (anti-EGFR), Besilesomab (anti-CEA), blinatumomab (anti-CD19), brentuximab (anti-CD30), Cantuzumab, Cibisatamab (anti-CEACAM5), Cirmtuzumab (anti-ROR1), cetuximab (anti-EGFR), Clivatuzumab (anti-MUC1), Gatipotuzumab (anti-MUC1), Coltuximab (anti-CD19), Daratumumab (anti-CD38), Duligotuzumab (anti-ERBB3), Edrecolomab (anti-EpCam), Enfortumab (anti-Nectin-4), Enoblituzumab (anti-CD276), Ensituximab (anti-MUC5AC), Epratuzumab (anti-CD22), Farletuzumab (anti-FOLR1), Igovomab (anti-CA-125), Inebilizumab (anti-CD19), Iratumumab (anti-CD30), Labetuzumab (anti-CEA), Margetuximab (anti-HER2/neu), Matuzumab (anti-EGFR), Modotuximab (anti-EGFR extracellular domain III), Naptumomab (anti-5T4), Naratuximab (anti-CD37), Necitumumab (anti-EGFR), Nimotuzumab (anti-EGFR), Obinutuzumab (anti-CD20), Ocaratuzumab (anti-CD20), Ocrelizumab (anti-CD20), Ofatumumab (anti-CD20), Otlertuzumab (anti-CD37), Ontuxizumab (anti-TEM1), Pertuzumab (anti-HER2/neu), Polatuzumab (anti-CD79b), Rituximab (anti-CD20), Rovalpituzumab (anti-DLL3), Sacituzumab (anti-TROP-2), Seribantumab (anti-ERBB3/HER3), Tafasitamab (anti-CD19), Tetulomab (anti-CD37), Timigutuzumab (anti-HER2), Trastuzumab (anti-HER2), Ublituximab (anti-CD20), or Zolbetuximab (anti-Claudin 18.2). In some embodiments, the aforementioned target-binding moieties (e.g. antibodies) have been recombinantly engineered to comprise at least one amino acid substitution Fc domain in which an amino acid of the Fc domain has been replaced by the amino acid cysteine. Said cysteine-engineered antibodies are particularly advantageous for use with the modular linker compound according to Formula (I) of the present disclosure, because they allow for a site-specific conjugation between the cysteine-engineered antibody and the modular linker compound of Formula (I) resulting homogenous antibody-drug conjugates with a degree of labelling of about 2 (e.g. from about 1.8, 1.9, 1.95 to about 2.0, 2.05, 2.1, 2.2, or from about 1.98, 2.0 to about 2.1, 2.15, 2.2). Corresponding protocols on the generation of cysteine-substituted or cysteine-engineered antibodies have been disclosed in WO2016040856A2, or in Junutula, et al., 2008b Nature Biotech., 26 (8): 925-932 (both documents incorporated by reference). The preferred cysteine substitution in the Fc region of the antibodies as disclosed above is D265C IgG1 Fc domains or corresponding positions in IgG2, IgG4 antibodies (numbering according to EU numbering system, Edelman et al., Proc Natl Acad Sci USA. 1969 May; 63 (1): 78-85) as disclosed in WO2016142049A1.

Optionally, the above antibodies or target-binding moieties have been engineered to further comprise the amino acid substitutions L234A, L235A (numbering according to EU numbering system) which reduce the interaction of the Fc domain of the respective antibody with the FcgRI, FcgRIIa, FcgRIIb and FcgRIIIa receptors. In some embodiments, the above antibodies may comprise amino acid substitutions such as N297A, P329G, G236R, or L234F, L235E in combination with D265C that reduce the interaction with the FcgRI, FcgRIIa, FcgRIIb and FcgRIIIa receptors.

In a fourth aspect, the target-binding drug conjugate of the third aspect is for use as a medicament, optionally for use in the treatment of cancer.

Accordingly, this fourth aspect of the present disclosure also encompasses methods of treating cancer, wherein the method comprises administering the target-binding drug conjugate of the third aspect to a subject in need thereof and/or use of the target-binding drug conjugate of the third aspect in the manufacture of a medicament for the treatment of cancer.

In some embodiments, the cancer is typically characterized by the expression of at least one of the cancer-specific or cancer-associated antigens described above.

Accordingly, in a fifth aspect, the present disclosure relates to a pharmaceutical composition comprising the target-binding drug conjugate according to the third or fourth aspect.

In a further aspect, the present disclosure also relates to a modular linker compound, optionally of the first aspect, when synthesized by the method of the second aspect.

In yet a further aspect, the present disclosure also relates to use of a modular linker compound according to the first aspect in the manufacture of a target-binding drug conjugate according to the fourth aspect.

### BRIEF DESCRIPTION OF THE DRAWINGS

Embodiments of the present disclosure will now be described, by way of example only, with reference to the accompanying drawings in which:

FIG. 1 In vitro cytotoxicity of linker-payload conjugate (IV.1) on (A) SKBR3 cells, (B) JIMT-1 cells. T-LALA-D265C denotes a trastuzumab variant which comprises the L234A, L235A and D265C mutations in its Fc region (numbering according to EU numbering system). T-LALA-D265C-va-PAB-exa denotes a control DAR2 exatecan ADC which comprises a self-immolative valine-alanine dipeptide linker lacking a solubility enhancing group.

FIG. 2 In vitro cytotoxicity of anti-GCC ADCs comprising the linker-payload conjugates (IV.4), (IV.6) on (A) HEK cells expressing human GUCY2C (HEK293-GUCY2C-HDP-2B3) cells, or (B) HEK wildtype cells. The cysteine adduct of linker payload (IV.6) was used as free compound.

FIG. 3 In vitro cytotoxicity of anti-GCC ADCs comprising the linker-payload conjugates (IV.5), (IV.6) on (A) HEK cells expressing human GUCY2C (HEK293-GUCY2C-HDP-2B3 cells, or (B) HEK293 wildtype cells. SEC indicates purification of the ADC by size-exclusion chromatography.

FIG. 4 In vitro cytotoxicity of anti-GCC ADCs comprising the linker-payload conjugates (IV.8), (IV.17) and (IV.19) on (A) HEK cells overexpressing human GUCY2C (HEK293-GUCY2C-HDP-2B3) cells, or (B) HEK293 wildtype cells. SEC indicates purification of ADC by size-exclusion chromatography. “N/A” indicates that no EC50 calculation was done.

FIG. 5 In vitro cytotoxicity of anti-GCC ADCs comprising the linker-payload conjugates (IV.9), (IV.12) and (IV.18) on (A) HEK cells overexpressing human GUCY2C (HEK293-GUCY2C-HDP-2B3) cells, or (B) HEK293 wildtype cells.

FIG. 6 In vitro cytotoxicity of anti-GCC ADCs comprising the linker-payload conjugates (IV.9), (IV.12) and (IV.18) on (A) HEK cells overexpressing human GUCY2C (HEK293-GUCY2C-HDP-2B3 cells, or (B) HEK293 wildtype (wt) cells.

FIG. 7 In vitro cytotoxicity of anti-GCC ADCs comprising the linker-payload conjugates (IV.18), (IV.16) and (IV.6) on (A) HEK cells overexpressing human GUCY2C (HEK293-GUCY2C-HDP-2B3 cells, or (B) HEK293 wildtype cells. SEC indicates purification of ADC by size-exclusion chromatography.

FIG. 8 shows mean tumor volume [mm³] depicted from day 0 to day 94 post group allocation in mice treated either with vehicle control, with the Exatecan-TBDC of the present disclosure IV.6 or with an Deruxtecan-ADC, as a single dose of 25 mg/kg on day 0 or 1×/week for 3 weeks (n=10 animals/group, mean depicted±SD). “hD” denotes high DAR (DAR10) ADCs. Exatecan-TBDC-IV.6 administered as single dose or multiple doses is equally effective in reducing tumor burden in the animals compared to a hD deruxtecan-based ADC against the same target.

FIG. 9 shows mean tumor volume [mm³] depicted from day 0 to day 67 post group allocation in mice treated either with vehicle control or with the Exatecan-TBDCs of the present disclosure IV.2 and IV.11, as a single dose of 15 mg/kg on day 1 (n=10 animals/group, mean depicted±SD).

FIG. 10 shows mean tumor volume [mm³] depicted from day 0 to day 61 post group allocation in mice treated either with vehicle control or with the Exatecan-TBDC of the present disclosure IV.6 as a single dose of 15 mg/kg on day 1 (n=10 animals/group, mean depicted±SD).

FIG. 11 shows mean tumor volume [mm³] depicted from day 0 to day 70 post group allocation in mice treated either with vehicle control or with the Exatecan-TBDC of the present disclosure IV.2 20 mg/kg, either as a single dose or multiple doses (n=9 animals/group, mean depicted±SD).

FIG. 12 shows mean tumor volume [mm³] depicted from day 0 to day 70 post group allocation in mice treated either with vehicle control or with the Exatecan-TBDC of the present disclosure IV.5 20 mg/kg, either as a single dose or multiple doses (n=9 animals/group, mean depicted±SD).

FIG. 13 shows mean tumor volume [mm³] depicted from day 0 to day 74 post group allocation in mice treated either with vehicle control or with the Exatecan-TBDCs of the present disclosure IV.16 or IV.3 15 mg/kg as a single dose (n=10 animals/group, mean depicted±SD).

FIG. 14 shows mean tumor volume [mm³] depicted from day 0 to day 66 post group allocation in mice treated either with vehicle control or with the Exatecan-TBDC of the present disclosure IV.9 either as a single dose of 15 mg/kg on day 1 or 1×/week for 3 weeks with 10 or 15 mg/kg (n=10 animals/group, mean depicted±SD).

FIG. 15 shows mean tumor volume [mm³] depicted from day 0 to day 66 post group allocation in mice treated either with vehicle control or with the Exatecan-TBDCs of the present disclosure IV.1 or IV.20 as a single dose of 15 mg/kg on day 1 (n=10 animals/group, mean depicted±SD).

FIG. 16 shows mean tumor volume [mm³] depicted from day 0 to day 66 post group allocation in mice treated either with vehicle control or with the Exatecan-TBDC of the present disclosure IV.8 as a single dose of 15 mg/kg on day 1 (n=10 animals/group, mean depicted #SD).

FIG. 17 shows mean tumor volume [mm³] depicted from day 0 to day 66 post group allocation in mice treated either with vehicle control or with the Exatecan-TBDC of the present disclosure IV.18 as a single dose of 15 mg/kg on day 1 (n=10 animals/group, mean depicted #SD).

FIG. 18 shows mean tumor volume [mm³] depicted from day 0 to day 22 post group allocation in mice treated either with vehicle control or with the Exatecan-TBDCs of the present disclosure IV.9, IV.8, IV.12 or IV.18 or IV.20 1×/week for 3 weeks with 5 mg/kg each (n=10 animals, mean depicted±SD). “hD” denotes high DAR (DAR10). Exatecan-TBDCs of the present disclosure comprising compounds (IV.9), (IV.12), (IV.18), and (IV.22) reduce tumor burden more efficiently than a hD deruxtecan-based ADC against the same target at the same total dose of ADC (or Exatecan-TBDCs) administered, while Exatecan-TBDCs of the present disclosure comprising compound (IV.8) is equally efficacious. This indicates that a lower dose of (IV.8), (IV.9), (IV.12), (IV.18), and (IV.22) is equally or more efficacious in reducing tumor burden in the animals than a higher dose of deruxtecan-payload.

FIG. 19 shows mean tumor volume [mm³] depicted from day 0 to day 22 post group allocation in mice treated either with vehicle control or with the Exatecan-TBDCs of the present disclosure IV.9, IV.8, IV.12 or IV.18 or IV.20 1×/week for 3 weeks with 2.5 mg/kg each (n=10 animals, mean depicted±SD). The results indicate that a lower dose of (IV.8), (IV.9), (IV.12), (IV.18), and (IV.22) is equally or more efficacious in reducing tumor burden in the animals than a higher dose of deruxtecan-payload.

FIG. 20 shows mean tumor volume [mm³] depicted from day 0 to day 100 post group allocation, measured by caliper. Mice were treated with vehicle control, IV.9, IV.8, IV.12, IV.18 or IV.22 1×/week for 3 weeks with 5 mg/kg each (n=10 animals, mean depicted±SD).

FIG. 21 shows mean tumor volume [mm³] depicted from day 0 to day 100 post group allocation, measured by caliper. Mice were treated with vehicle control, IV.9, IV.8, IV.12, IV.18 or IV.22 1×/week for 3 weeks with 2.5 mg/kg each (n=10 animals, mean depicted±SD).

FIG. 22 shows overall survival depicted from day 0 to day 100 post group allocation. Survival was monitored daily. Mice were treated with vehicle control, IV.9, IV.8, IV.12, IV.18 or IV.22 1×/week for 3 weeks with 5 mg/kg each (n=10 animals, mean depicted±SD).

FIG. 23 shows overall survival depicted from day 0 to day 100 post group allocation. Survival was monitored daily. Mice were treated with vehicle control, IV.9, IV.8, IV.12, IV.18 or IV.22 1×/week for 3 weeks with 2.5 mg/kg each (n=10 animals, mean depicted±SD).

FIG. 24 shows mean tumor volume [mm³] depicted from day 0 to day 87 post group allocation, measured by caliper. Mice were treated with vehicle control, IV.9, IV.8, IV.12, IV.18 or IV.22 with a single intravenous dose of 10 mg/kg (n=10 animals, mean depicted±SD).

FIG. 25 shows mean tumor volume [mm³] depicted from day 0 to day 87 post group allocation, measured by caliper. Mice were treated with vehicle control, IV.9, IV.8, IV.12, IV.18 or IV.22 with a single intravenous dose of 5 mg/kg (n=10 animals, mean depicted±SD).

FIG. 26 shows overall survival depicted from day 0 to day 87 post group allocation. Survival was monitored daily. Mice were treated with vehicle control, IV.9, IV.8, IV.12, IV.18 or IV.22 with a single intravenous dose of 10 mg/kg (n=10 animals, mean depicted±SD).

FIG. 27 shows overall survival depicted from day 0 to day 87 post group allocation. Survival was monitored daily. Mice were treated with vehicle control, IV.9, IV.8, IV.12, IV.18 or IV.22 with a single intravenous dose of 5 mg/kg (n=10 animals, mean depicted±SD).

FIG. 28 shows that in a 96 h BrdU ELISA on Her2 expressing NCI-N87 cells, anti-Her2 conjugates showed full blown cytotoxicity with EC50 values between 1.749×10-11 M (T-LALA-D265C-Compound IV.27) and 3.279×10-12 M (T-LALA-D265C-Compound IV.25).

FIG. 29 shows that in a 96 h BrdU ELISA on target negative MDA-MB-231 cells, anti-Her2 conjugates with no cytotoxicity.

FIG. 30 shows that in a coculture of Trop2 expressing NCI-N87 cells and TLR7 expressing reporter cell line (HEK-hTLR7), both anti-Trop2 TLR7 conjugates showed strong and dose dependent TLR7 activation.

FIG. 31A shows conjugates produced with Linker/Payload (L/P) with solubility enhancer exhibited much fewer aggregates in the elution profile. The derivatives were purified by preparative FPLC using HiLoad 16/600 Superdex 200 pg prepacked XK16 columns, equilibrated with 1×PBS, pH7.4, with a flow rate of 1.6 mL/min. The elution profile at 280 nm shows a main peak corresponding to monomers eluting at around 80 min, while aggregates and other high molecular weight species (HMWS) elute earlier, between 60 and 70 min.

FIG. 31B shows conjugates produced with Linker/Payload (L/P) without solubility enhancer. The derivatives were purified by preparative FPLC using HiLoad 16/600 Superdex 200 pg prepacked XK16 columns, equilibrated with 1×PBS, pH7.4, with a flow rate of 1.6 mL/min. The elution profile at 280 nm shows a main peak corresponding to monomers eluting at around 80 min, while aggregates and other high molecular weight species (HMWS) elute earlier, between 60 and 70 min.

FIG. 32 shows the conjugation taking place by coupling a terminal thiol-reactive moiety such as a maleimide residue of the drug-carrying linker (“linker-payload”) to the free SH group of a genetically-engineered cysteine residue in the Fc region of the antibody.

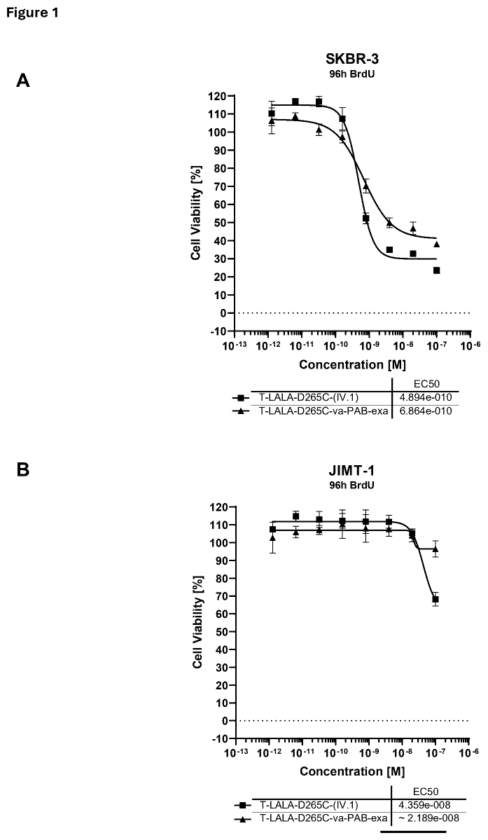

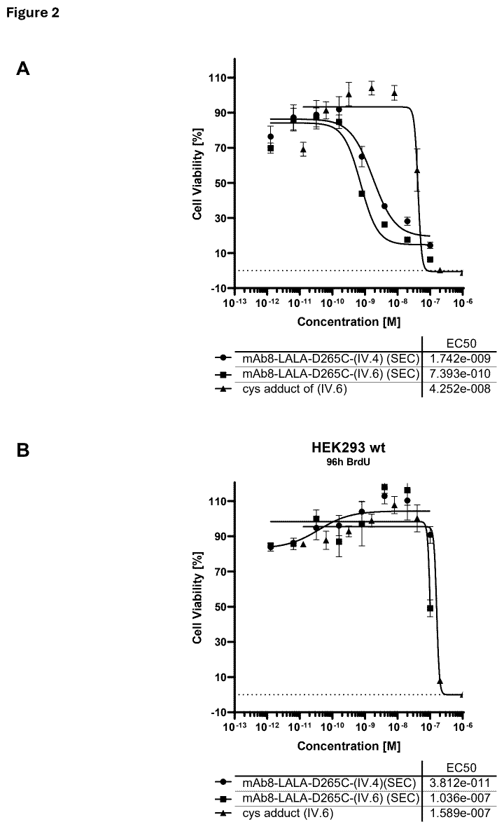

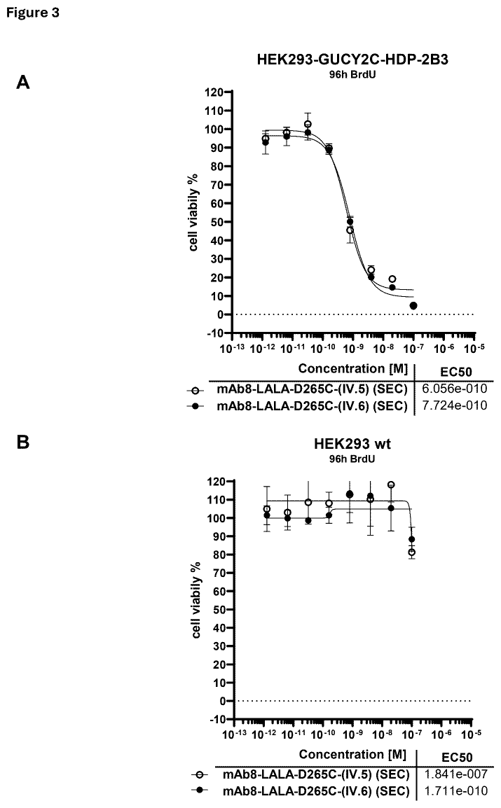

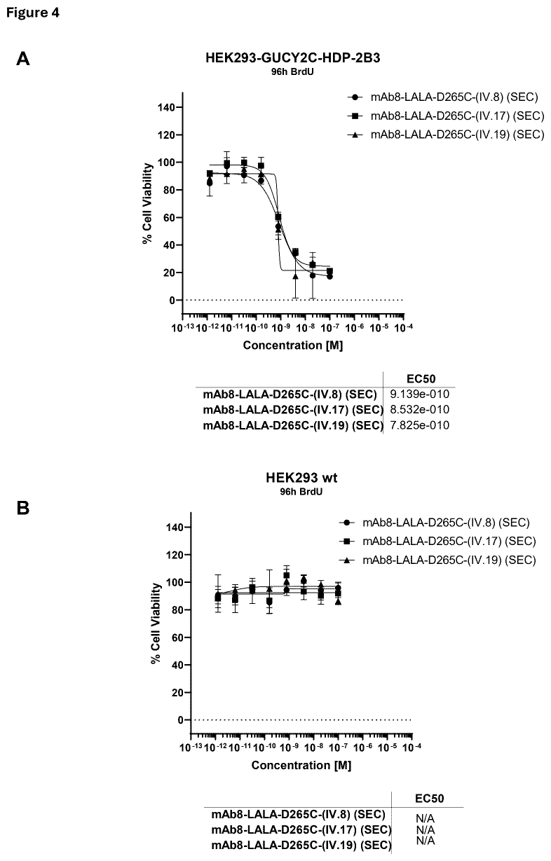

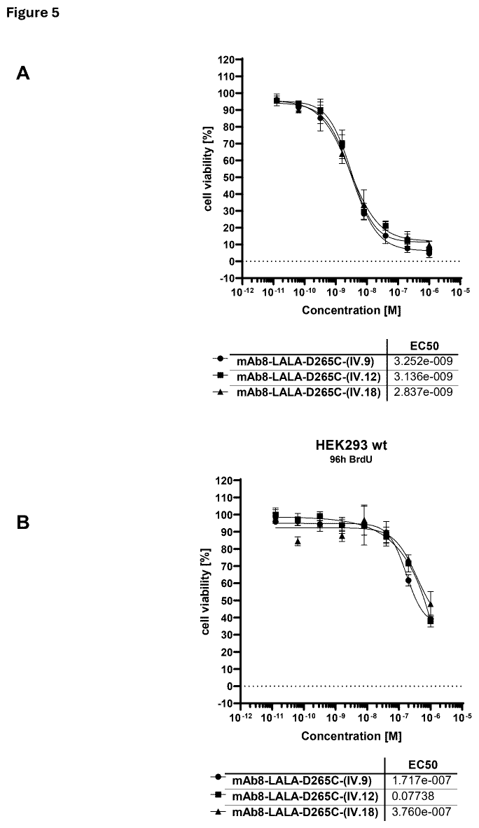

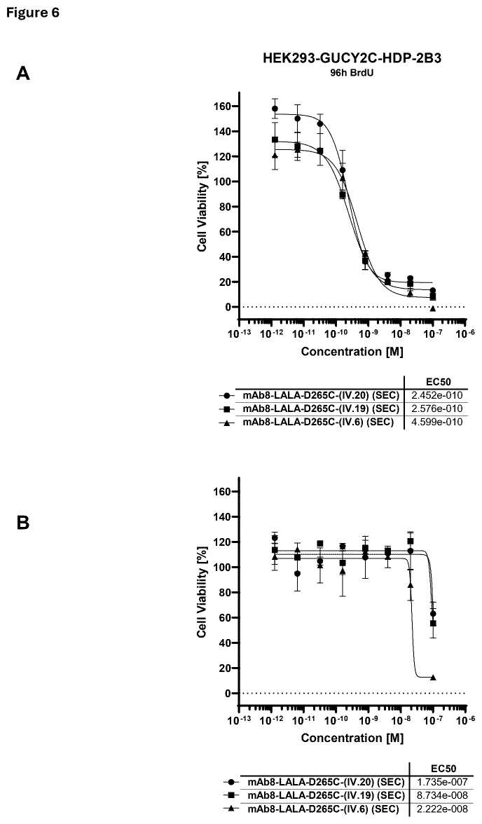

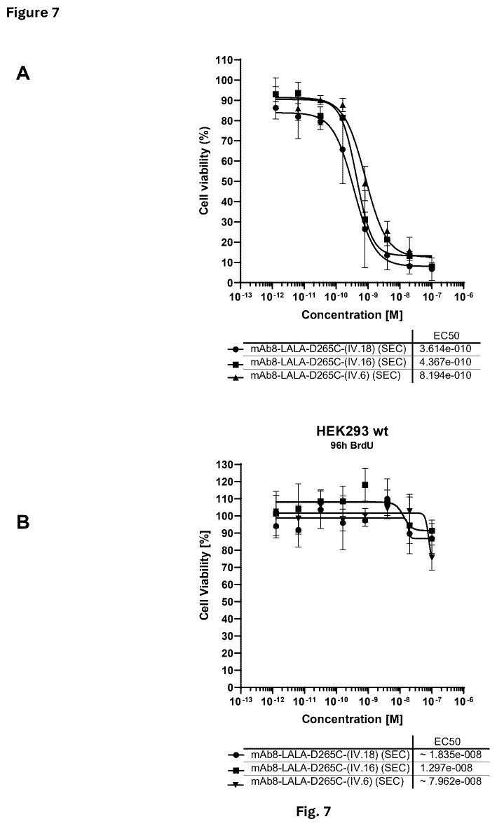

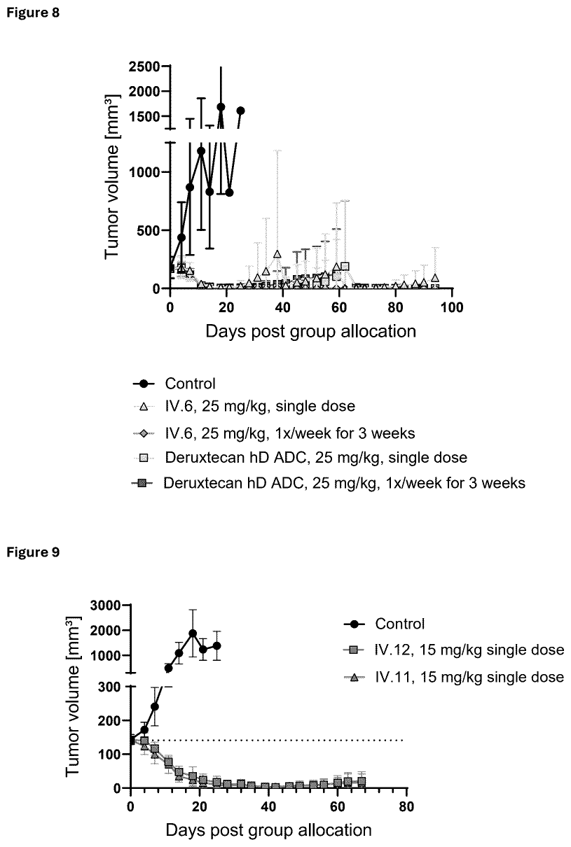

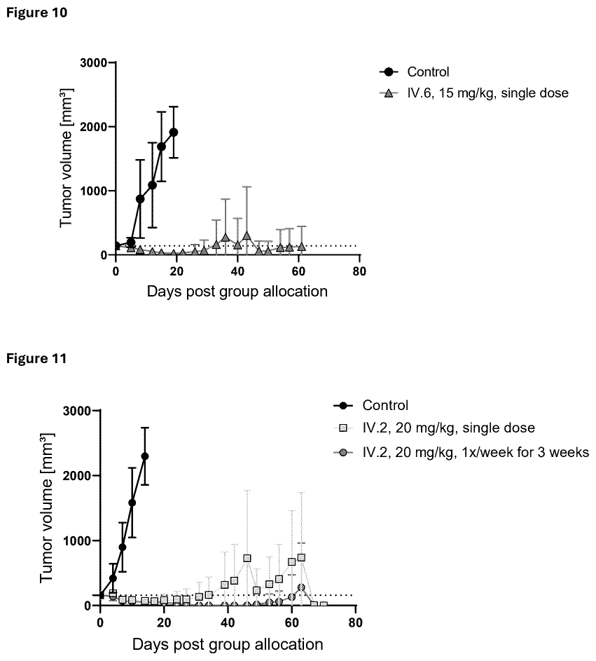

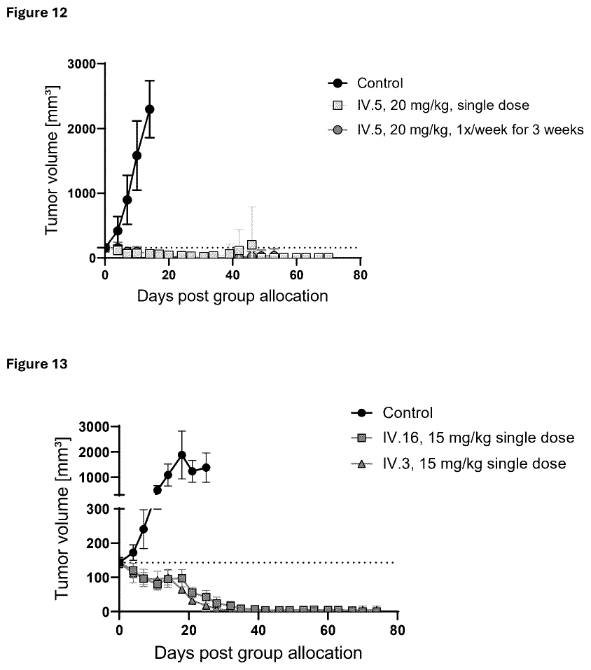

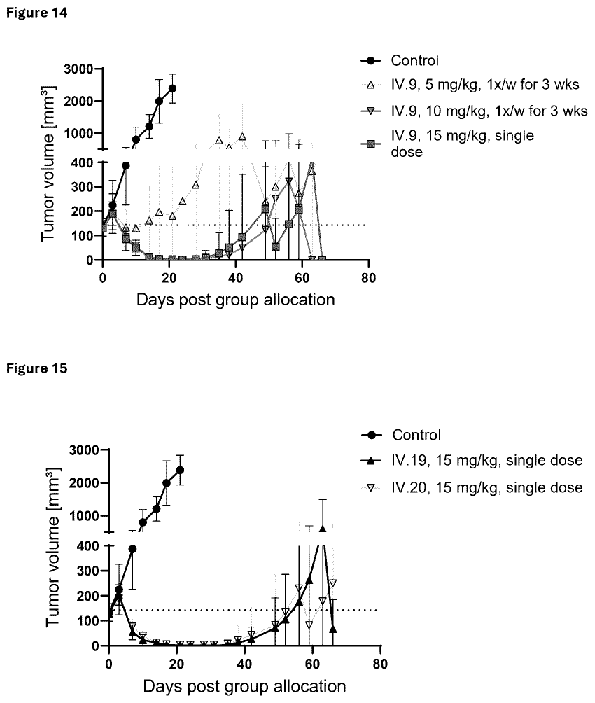

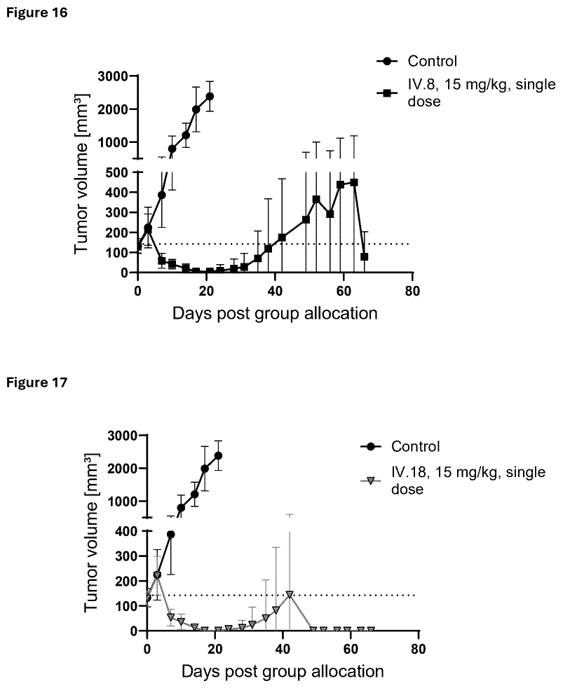

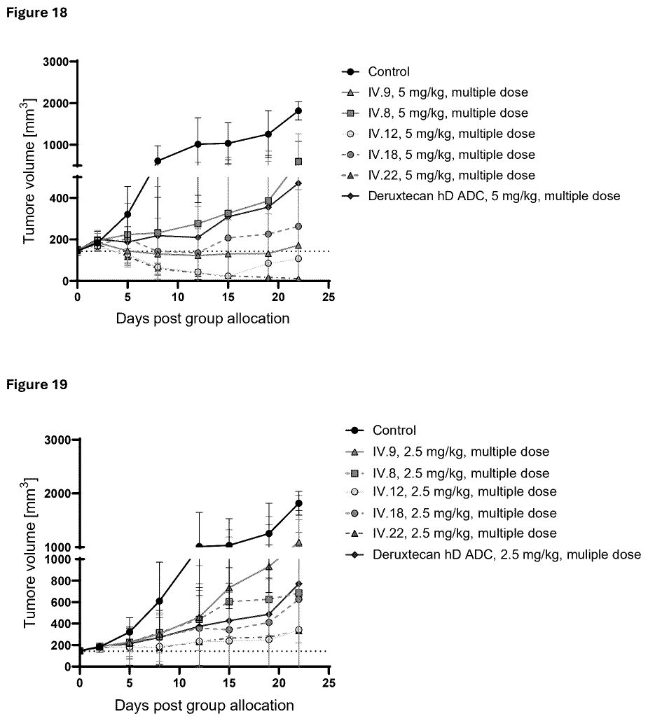

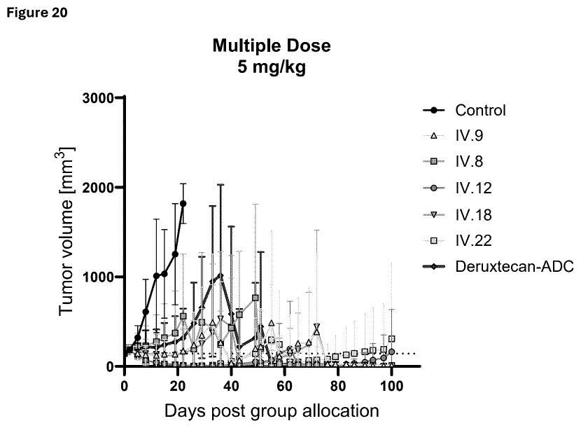

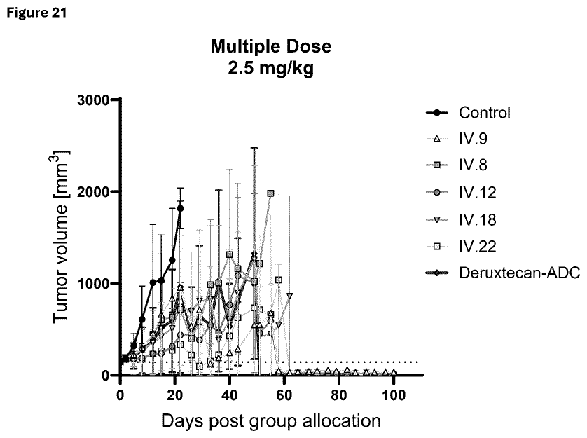

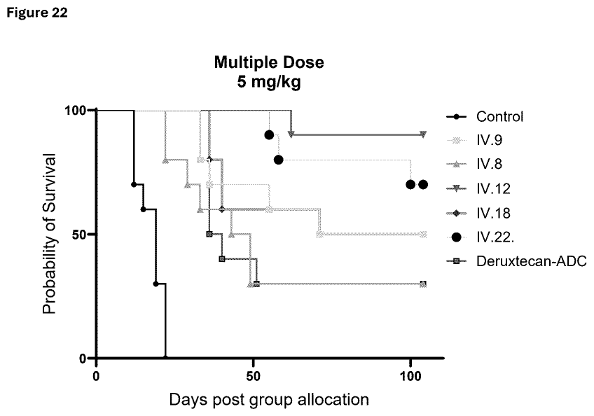

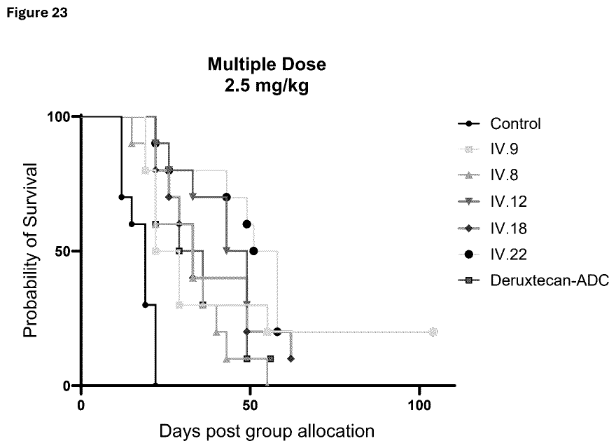

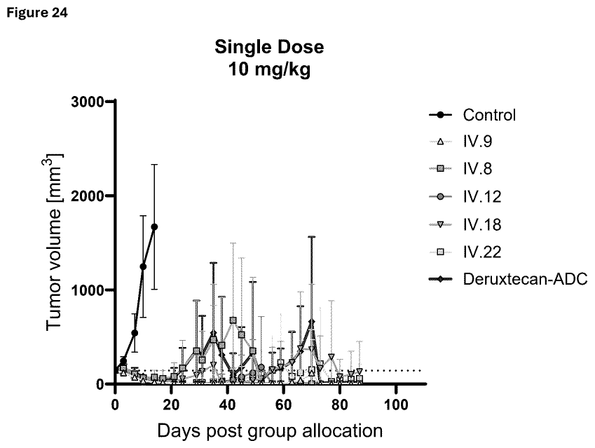

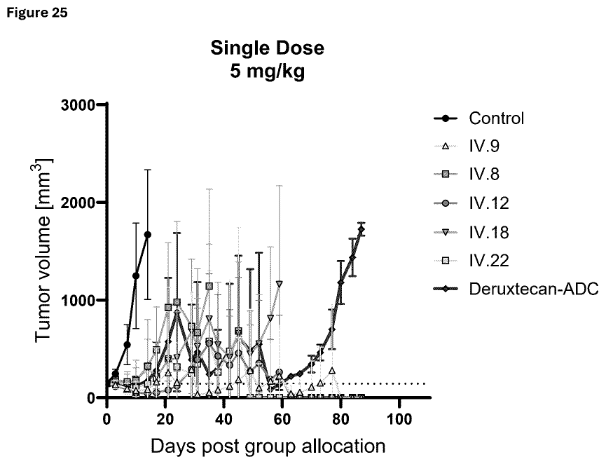

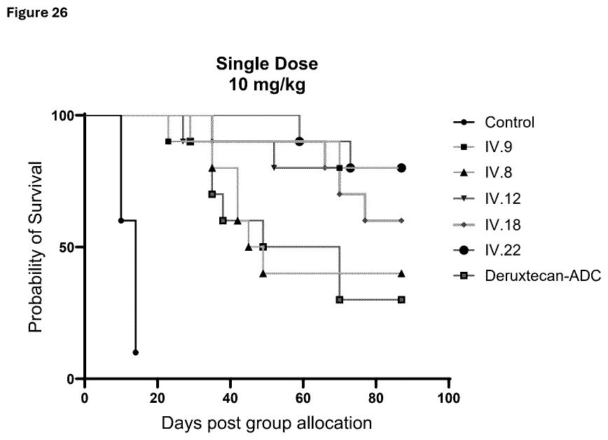

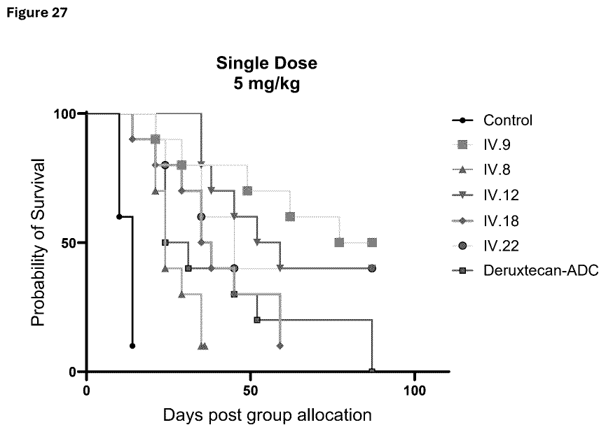

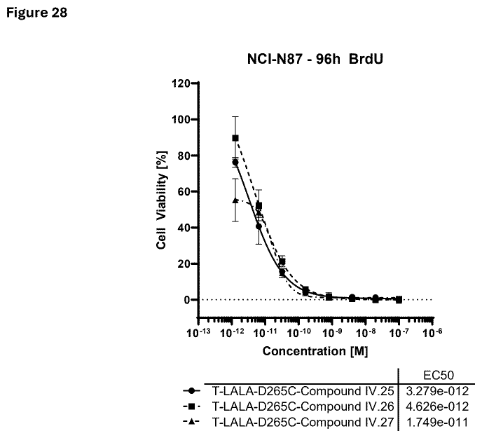

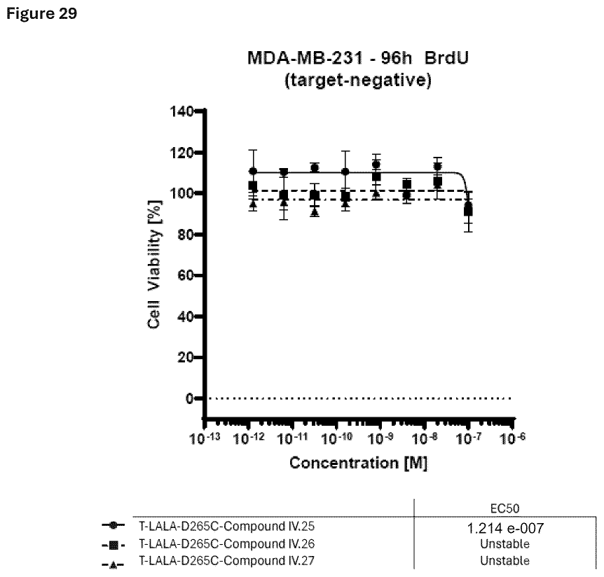

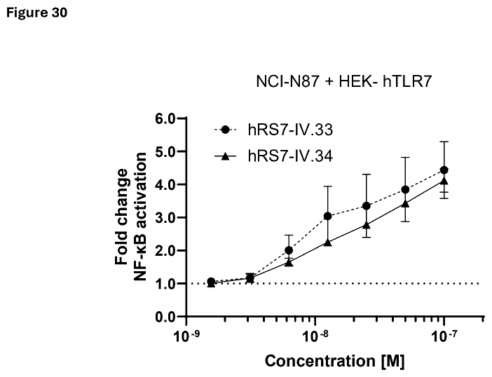

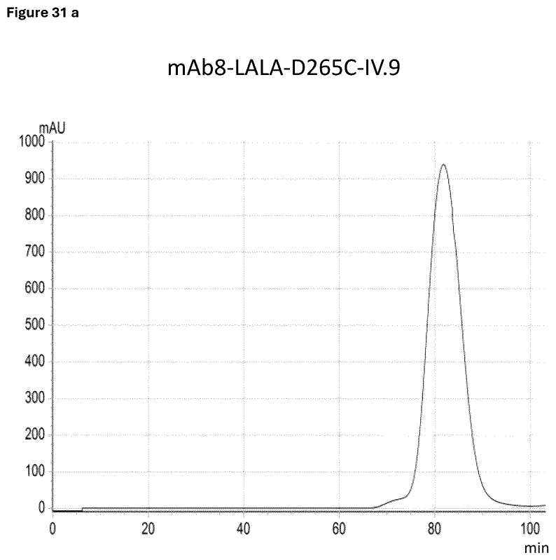

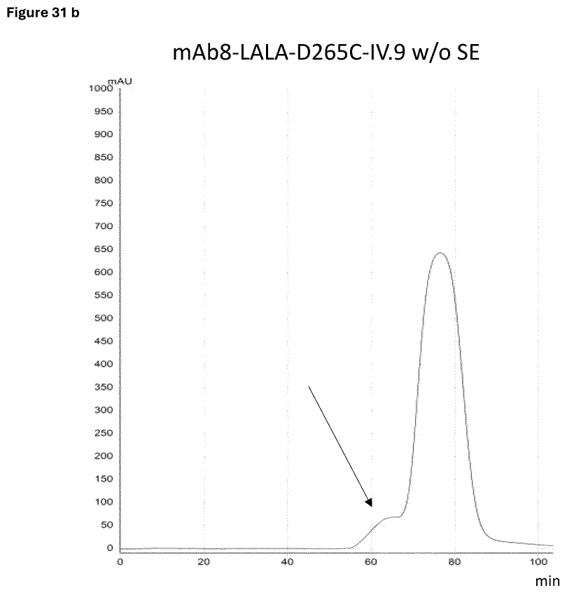

### DETAILED DESCRIPTION

Before the present disclosure is described in detail below, it is to be understood that this disclosure is not limited to the particular methodology, protocols and reagents described herein as these may vary. It is also to be understood that the terminology used herein is for the purpose of describing particular embodiments only and is not intended to limit the scope of the present disclosure which will be limited only by the appended claims. Unless defined otherwise, all technical and scientific terms used herein have the same meaning as commonly understood by one of ordinary skill in the art.

Optionally, the terms used herein are defined as described in “A multilingual glossary of biotechnological terms: (IUPAC Recommendations)”, Leuenberger, H. G. W, Nagel, B. and Kolbl, H. eds. (1995), Helvetica Chimica Acta, CH-4010 Basel, Switzerland. The definitions of the chemical groups as used herein unless specified otherwise shall have the meaning and be defined as provided in “Compendium of Chemical Terminology” (“Gold Book”) published by the International Union of Pure and 13 Applied Chemistry (IUPAC) version 2.3.3, goldbook.iupac.org, ISBN: 0-9678550-9-8), the content of which is hereby incorporated by reference.

Notwithstanding, in order to provide a clear and consistent understanding of the specification and claims, as well as of the scope to be given to certain terms, the following definitions are provided.

### Definitions

The term “modular linker compound” in the context of the present disclosure refers to a linker that comprises at least one molecule of a cytotoxic drug substance (also referred to simply as drug or payload), several further structural as well as functional modules (also referred to as spacers or functional units or groups), including at least one solubility enhancing group and a branching unit, as well as a reactive group suitable to stably connect the modular linker compound to a target-binding moiety such as an antibody or a TCR.

The term “solubility enhancing group” in the context of the present disclosure refers to a module within the modular linker compound, which at least counters—at best negates—hydrophobic/lipophilic properties of a cytotoxic drug substance such as to provide the modular linker compound comprising a specific payload with a high degree of aqueous solubility. A variety of solubility enhancing groups suitable for the incorporation into the modular linker compound of the present disclosure are well-known to the skilled person and include, without limitation: (a) polar functional groups, such as hydroxyl (—OH), amine (—NH₂), and carboxyl (—COOH) groups; polar sulfonate (—SO₃H) groups; polymeric chains, such as hydrophilic polyethylene glycol (PEG) chains; surfactant groups, such as alkyl chains; aromatic ring structures; halogen substituents such as the addition of fluorine or chlorine; ester (—COO—) or ether (—O—) groups; and cyclodextrins/cyclic oligosaccharides.

In the context of the present disclosure, the term “polyalcohol(s) made from carbohydrate(s)” relates to a group of carbohydrate-derived alcohols. The term “polyalcohol”, “polyol” and “sugar alcohols” are used synonymously.

In the context of the present disclosure, the term “PEG” refers to polyethylene glycol, the term “(PEG)₁₋₁₀” refers to a linker with 1 to 10 ethylene glycol moieties,

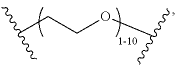

e.g. 1, 2, 3, 4, 5, 6, 7, 8, 9 and 10 ethylene glycol moieties.
The term “C₂-amide-PEG₁₋₁₀” refers to a linker with 1 to 10 ethylene glycol moieties covalently attached to a propylamide group,

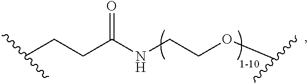

e.g. 1, 2, 3, 4, 5, 6, 7, 8, 9 and 10 ethylene glycol moieties.

In the context of the present disclosure, the term “self-immolative spacer” refers to a spacer that is stably connected to the cytotoxic drug substance but undergoes triggered self-immolation, i.e. decomposition, such that its connection to the cytotoxic drug substance is abolished and the drug substance is released without any adducts or traces of the self-immolative spacer itself. In some embodiments, a change in pH can trigger self-immolation of the spacer and release of the payload. In other embodiments, the self-immolative spacer is also connected to an enzymatically cleavable peptide sequence and enzymatic cleavage of the peptide triggers self-immolation of the spacer and release of the payload. An illustrative example of an enzymatically triggered self-immolative spacer is a p-aminobenzyl (PAB) spacer connected to an enzymatically cleavable Val-Ala dipeptide.

In the context of the present disclosure, the term “cleavable linker compound” refers to a linker compound that is cleavable (i) by an enzyme, or (ii) in a reducing environment. Further, the terms “enzymatically cleavable” or “cleavable by an enzyme” or the likes convey that the peptide, structure, sequence or spacer in question can be cleaved by an enzyme, particularly by a lysosomal protease, such as Cathepsin B, resulting in the release of the cytotoxic drug substance/payload.

In the context of the present disclosure, the term “branching unit” refers to a structure within a modular linker compound that allows for a furcation of the compound into a branched structure, wherein each branch is typically connected to at least one molecule of the cytotoxic drug substance via a self-immolative spacer. Therefore, a modular linker compound typically comprises as many drug molecules as branches. As such, a modular linker compound comprising between 1 and 10, e. g., 1, 2, 3, 4, 5, 6, 7, 8, 9 and 10, optionally between 2 and 8, 2 and 6 or 2 and 4 branches, typically comprises between 1 and 10, e. g. 1, 2, 3, 4, 5, 6, 7, 8, 9 and 10, optionally between 2 and 8, 2 and 6 or 2 and 4 cytotoxic drug substance molecules, respectively.

In the context of the present disclosure, the term “reactive moiety for conjugation to a target-binding moiety” refers to a chemically reactive group within the linker that can react with a correspondingly reactive group of the target-binding moiety under suitable conditions such that the resulting reaction product covalently conjugates the remaining linker compound to the target-binding moiety.

In the context of the present disclosure, the term “reaction product of the modular linker compound and a correspondingly reactive group of a target-binding moiety” refers a moiety formed as the result of a reaction between the reactive moiety of the modular linker compound of the present disclosure and a correspondingly reactive group of a target-binding moiety. For example, if the reactive moiety of the modular linker compound of the present disclosure is a maleimide, the reaction product resulting from the reaction of the maleimide with the sulphur atom of the cysteine residue of the target-binding moiety, is a succinimidyl thioether or a derivative thereof. Further, if the reactive moiety of the modular linker compound of the present disclosure is a group that selectively reacts with the thiol group of a free cysteine of the target-binding moiety, i.e. if the reactive moiety of the modular linker compound is a “thiol-reactive moiety”, the reaction product is typically selected from: thiol-substituted acetamide; thiol-substituted succinimide; thiol-substituted succinamic acid; thiol-substituted heteroaryl, particularly thiol-substituted benzothiazole, thiol-substituted phenyltetrazole and thiol-substituted phenyloxadiazole; and a disulphide, wherein one sulphur atom is derived from a cysteine residue of the target-binding moiety.

In the context of the present disclosure, the term “target-binding moiety” refers to a polypeptide molecule that specifically recognizes and binds a target sequence or epitope of an antigen relying on the immunoglobulin (Ig) concept of target/epitope recognition known from molecules of the conventional Ig format (i.e. IgG, IgD, IgE, IgA and/or IgM). Accordingly, in particular embodiments, a target-binding moiety may comprise, at least, complementarity-determining regions (CDRs) of an Ig sufficient to confer target/epitope specificity and, in some instances, the target-binding moiety may comprise both variable heavy (V_{H}) and variable light (V_{L}) chain sequences of an Ig to confer target/epitope specificity. In some embodiments, a target-binding moiety may be an Ig as such, e.g. an IgG. This, however, does not mean that the target-binding moiety must still be in this Ig format. Instead, the target-binding moiety can also be an Ig fragment or derivative thereof, e.g. a single-chain variable fragment (scFv) or an antigen-binding fragment (Fab-obtained through papain cleavage of an IgG; F(ab′)—obtained through pepsin cleavage of an IgG; and/or F(ab′)2—obtained pepsin cleavage of an IgG and subsequent β-mercaptoethanol treatment; etc.). Likewise, immunoreactive molecules of the present disclosure can be a new antibody format, for example and without limitation, a bi- or tri-specific antibody construct, a Diabody, a Camelid Antibody, a Domain Antibody, a Nanobody, a bivalent homodimer with two chains consisting of scFvs, a shark antibody, an antibody consisting of new-world primate framework plus non-new world primate CDR or a dimerised construct comprising CH₃+V_{L}+V_{H}.

In particular embodiments, a target-binding moiety includes T cell receptors (TCRs). TCRs are disulfide-linked membrane-anchored heterodimeric proteins that normally consist of the highly variable alpha (a) and beta (b) chains expressed as part of a complex with the invariant CD3 chain molecules on the surface of T cells. An important step in the process of forming the TCR heterodimer is called “pairing”. T cells expressing paired receptors are referred to as a: b (or ab) T cells, although a minority of T cells express an alternate receptor, formed by variable gamma (g) and delta (d) chains, referred to as gd T cells. Each chain of the TCR is composed of two extracellular domains: A variable (V) region and a constant (C) region, both belonging to the immunoglobulin superfamily (IgSF) domain forming antiparallel b-sheets. The constant region is proximal to the cell membrane, followed by a transmembrane region and a short cytoplasmic tail, while the variable region binds to the peptide/MHC complex of an antigen presenting cell.

As used herein, a target-binding moiety is considered to “specifically recognise and bind” a target sequence or epitope of an antigen, if it has a dissociation constant KD to the antigen of 100 μM or less, optionally 50 μM or less, optionally 30 μM or less, optionally 20 μM or less, optionally 10 μM or less, optionally 5 μM or less, optionally 1 μM or less, optionally 900 nM or less, optionally 800 nM or less, optionally 700 nM or less, optionally 600 nM or less, optionally 500 nM or less, optionally 400 nM or less, optionally 300 nM or less, optionally 200 nM or less, even optionally 100 nM or less, even optionally 90 nM or less, even optionally 80 nM or less, even optionally 70 nM or less, even optionally 60 nM or less, even optionally 50 nM or less, even optionally 40 nM or less, even optionally 30 nM or less, even optionally 20 nM or less, and even optionally 10 nM or less.

In the context of the present disclosure, the term a “pharmaceutically acceptable salt or solvates” includes acid addition salts, formed with inorganic acids such as hydrochloric acid, hydro bromic acid, sulphuric acid, nitric acid, phosphoric acid, and the like; or formed with organic acids such as glycolic acid, pyruvic acid, lactic acid, malonic acid, malic acid, inaleic acid, fumaric acid, tartaric acid, citric acid, 3-(4-hydroxybenzoyl)benzoic acid, cinnamic acid, mandelic acid, methane sulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, lauryl sulphuric acid, gluconic acid, glutamic acid, salicylic acid, muconic acid, and the like. The term further includes basic addition salts formed with the conjugate bases of any one of the above-listed inorganic acids, wherein the conjugate bases comprise a cationic component selected from Na⁺, K⁺, Mg²⁺, Ca²⁺, and NHgR′_{4-g}⁺; in which R′ is a C₁₋₃ alkyl and g is a number selected from among 0, 1, 2, 3, or 4 as well as solvent addition forms (i.e, solvates) of the respective acid addition salt.

As used herein, “treat”, “treating” or “treatment” of a disease or disorder means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

As used herein, a “patient” means any mammal or bird who may benefit from a treatment with the target-binding drug conjugates described herein. Optionally, a “patient” is selected from the group consisting of laboratory animals (e.g. mouse or rat), domestic animals (including e.g. guinea pig, rabbit, chicken, pig, sheep, goat, camel, cow, horse, donkey, cat, or dog), or primates including human beings. It is particularly preferred that the “patient” is a human being.

A “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

In addition to the above definitions, and unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

Further, reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to and are not intended to imply that the objects so described must be in a given sequence, either temporally, spatially, in ranking, or in any other manner.

As used herein, the term “exemplary” is used in the sense of providing examples, as opposed to indicating quality. That is, an “exemplary embodiment” is an embodiment provided as an example, as opposed to necessarily being an embodiment of exemplary quality.

As used herein, the partition coefficient, abbreviated PC, is defined as a particular ratio of the concentrations of a solute between two solvents (a biphase of liquid phases), specifically for un-ionized solutes, and the logarithm of the ratio is thus clog P. When one of the solvents is water and the other one is n-octanol, the log P value is a measure of lipophilicity or hydrophobicity. (see Comer J, Tam K (2001). “Lipophilicity Profiles: Theory and Measurement”, In Testa B, van de Waterbed H, Folkers G, Guy R, Comer J, Tam K (eds.). Pharmacokinetic Optimization in Drug Research: Biological, Physicochemical, and Computational Strategies. Weinheim: Wiley-VCH. pp. 275-304.)

Several documents are cited throughout the text of this specification. Each of the documents cited herein (including all patents, patent applications, scientific publications, manufacturer's specifications, instructions, GenBank Accession Number sequence submissions etc.), whether supra- or infra, is hereby incorporated by reference in its entirety to the extent possible under the respective patent law.

### Modular Linker Compounds

The inventors of the present disclosure have developed a modular linker compound that comprises modules (i.e. also referred to as groups, units, elements, etc.) which have been implicated with respect to target-binding drug conjugate (TBDC) function and efficacy, and which can readily be assembled in a combinatorial fashion to provide the modular linker compound.

The modular architecture of the modular linker compound allows for a high degree of flexibility with respect to selected modules such that the modular linker compound can accommodate various physico-chemical properties of relevant cytotoxic drug substances but also of the target-binding moiety to which the modular linker compound is to be conjugated. Therefore, it was found that, in a relatively simple fashion, the modular linker compound of the present disclosure allows for the generation a TBDC targeting a selected cancer-associated or cancer-specific antigen and comprising the cytotoxic drug substance of choice in the treatment of the particular cancer type.

In addition, and as will become apparent form the further description below, through the conjugation chemistries employed in the modular linker compound the generation of TBDCs with both homogeneous Degree-of-Labeling (DOL) and Drug-Antibody-Ratio (DAR) is readily achievable.

In a first aspect, the present disclosure relates to a modular linker compound of Formula (I)

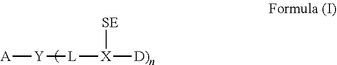

or a pharmaceutically acceptable salt or solvate thereof;
wherein:
X is a self-immolative spacer

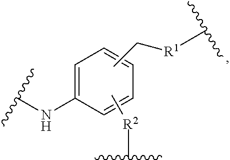

wherein:
R¹ covalently links X to D in para, meta or ortho to the amino group of X and is a bond,

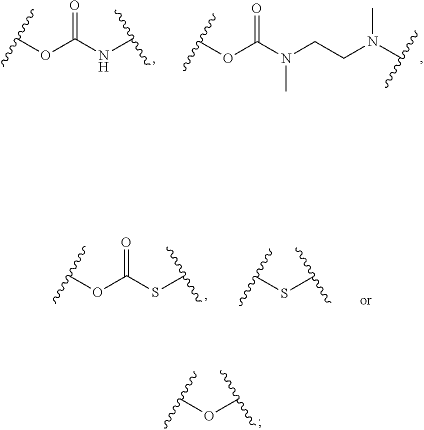

and
R² connects X to SE and is

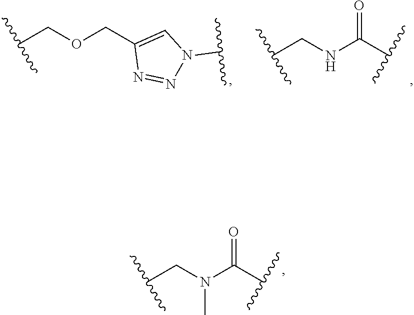

### or —O—;

D is a pharmaceutically active substance;
SE is a solubility enhancing group;
L is a spacer comprising a peptide covalently linked to X;
n is an integer of at least 2;
Y is a branching unit; and
A is a reactive moiety for conjugation to a target-binding moiety.

The component

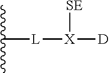

of Formula (I) is repeated at least twice in the modular linker compound of this first aspect (i.e. n is an integer of at least 2), but optionally between 2 and 5 times such as 2, 3, 4 or 5 times. This advantageous increase in the DOL of a single modular linker compound is dependent on the multimeric core of the compound defined by the degree of branching seen in the branching unit Y.

As defined above, the self-immolative spacer of the modular linker compound of the present disclosure is a para- or ortho-aminobenzyl spacer, which is stably connected to the cytotoxic drug substance D as well as to the solubility enhancing group SE, and which undergoes triggered self-immolation, i.e. decomposition, such that at least the cytotoxic drug substance D is released as a metabolite of the self-immolation reaction without any chemical moieties from the linker compound remaining attached to the drug. Specifically, in some embodiments, the amino group of a para-aminobenzyl spacer serves as the electron donor in the electron cascade of the 1,6-self-immolation reaction releasing the cytotoxic drug substance D. Advantageously, this traceless release mechanism ensures that the cytotoxic drug substance is not altered through its connection to and subsequent release from the linker compound.

In a specific embodiment of the first aspect, if the ClogP of the payload (D) is −1 or lower, the present disclosure relates to a modular linker compound of Formula (Ia)

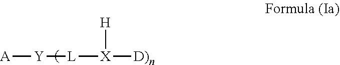

or a pharmaceutically acceptable salt or solvate thereof;
wherein:
X is a self-immolative spacer

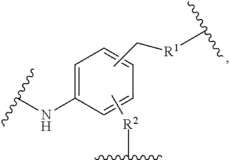

wherein:
R¹ covalently links X to D in para, meta or ortho to the amino group of X and is a bond,

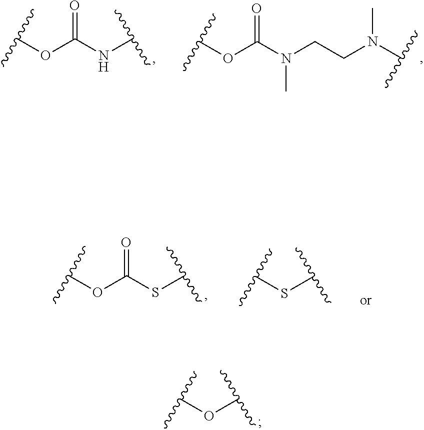

and
R² is H
D is a pharmaceutically active substance;
L is a spacer comprising a peptide covalently linked to X;
n is an integer of at least 2;
Y is a branching unit; and
A is a reactive moiety for conjugation to a target-binding moiety.

The component

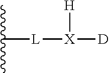

of Formula (Ia) is repeated at least twice in the modular linker compound of this first aspect (i.e. n is an integer of at least 2), but optionally between 2 and 5 times such as 2, 3, 4 or 5 times. This advantageous increase in the DOL of a single modular linker compound is dependent on the multimeric core of the compound defined by the degree of branching seen in the branching unit Y.

As defined above, the self-immolative spacer of the modular linker compound of the present disclosure is a para- or ortho-aminobenzyl spacer, which is stably connected to the cytotoxic drug substance D as well as to the solubility enhancing group SE, and which undergoes triggered self-immolation, i.e. decomposition, such that at least the cytotoxic drug substance D is released as a metabolite of the self-immolation reaction without any chemical moieties from the linker compound remaining attached to the drug. Specifically, in some embodiments, the amino group of a para-aminobenzyl spacer serves as the electron donor in the electron cascade of the 1,6-self-immolation reaction releasing the cytotoxic drug substance D. Advantageously, this traceless release mechanism ensures that the cytotoxic drug substance is not altered through its connection to and subsequent release from the linker compound.

In some embodiments, where the solubility enhancing group SE is also linked to in the para- or an ortho-position of the aminobenzyl spacer, the self-immolation reaction also leads to the release of the solubility enhancing group. However, as will be appreciated, in contrast to the release of the cytotoxic drug substance D, release of the solubility enhancing group is not of particular relevance to the functionality and efficacy of a TBDC based on the described modular linker compound. In contrast, a modular linker compound comprising the SE group linked to the self-immolative spacer in the meta position of the aminobenzyl spacer, and the cytotoxic drug substance linked in the adjacent para position of the aminobenzyl spacer. Without wanting to be bound by theory, the close proximity of the solubility enhancing group SE and the cytotoxic drug substance D is likely to lead to a better masking of the lipophilic/hydrophobic properties of the drug substance such as to further increase solubility of the modular linker compound.

In preferred embodiments, the immolative spacer X is one of X1.1 to X1.3, X2.1 to X2.3, X3.1 to X3.3 and X4.1 to X4.3 comprising one of the specific combinations of R¹ and R² as shown in the table directly below:

| immolative spacer X | R¹ (in para) | R² (in meta) |
|---|---|---|
| 1.1 | direct bond to D | 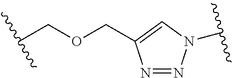 [SMILES: *COCc1cn(*)nn1] |
| 1.2 | direct bond to D | 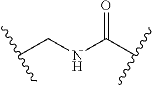 [SMILES: *CNC(*)=O] |
| 1.3 | direct bond to D | 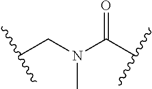 [SMILES: *CN(C)C(*)=O] |
| 2.1 | 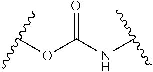 [SMILES: *NC(=O)O*] | 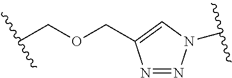 [SMILES: *COCc1cn(*)nn1] |
| 2.2 | 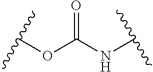 [SMILES: *NC(=O)O*] | 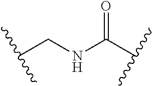 [SMILES: *CNC(*)=O] |
| 2.3 | 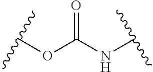 [SMILES: *NC(=O)O*] | 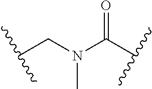 [SMILES: *CN(C)C(*)=O] |
| 3.1 | 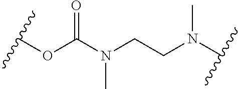 [SMILES: *OC(=O)N(C)CCN(*)C] | 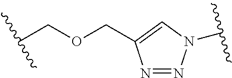 [SMILES: *COCc1cn(*)nn1] |
| 3.2 | 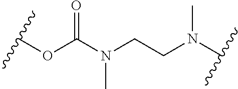 [SMILES: *OC(=O)N(C)CCN(*)C] | 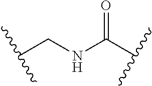 [SMILES: *CNC(*)=O] |
| 3.3 | 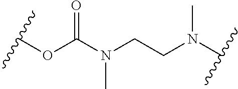 [SMILES: *OC(=O)N(C)CCN(*)C] | 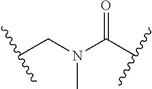 [SMILES: *CN(C)C(*)=O] |
| 4.1 | 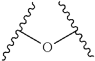 [SMILES: *O*] | 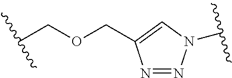 [SMILES: *COCc1cn(*)nn1] |
| 4.2 | 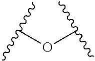 [SMILES: *O*] | 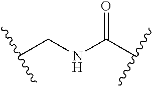 [SMILES: *CNC(*)=O] |
| 4.3 | 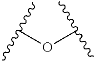 [SMILES: *O*] | 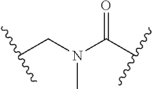 [SMILES: *CN(C)C(*)=O] |
| 5.1 | 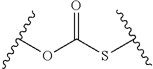 [SMILES: *OC(=O)S*] | 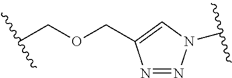 [SMILES: *COCc1cn(*)nn1] |
| 5.2 | 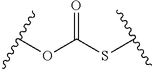 [SMILES: *OC(=O)S*] | 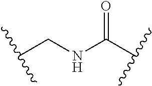 [SMILES: *CNC(*)=O] |
| 5.3 | 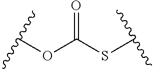 [SMILES: *OC(=O)S*] | 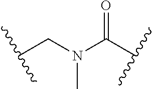 [SMILES: *CN(C)C(*)=O] |
| 6.1 | 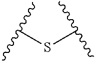 [SMILES: *S*] | 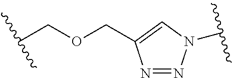 [SMILES: *COCc1cn(*)nn1] |
| 6.2 | 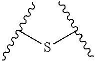 [SMILES: *S*] | 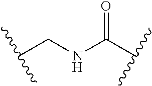 [SMILES: *CNC(*)=O] |
| 6.3 | 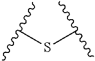 [SMILES: *S*] | 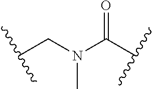 [SMILES: *CN(C)C(*)=O] |
| Immolative spacer X | R¹ (in meta) | R² (in ortho) |
| 7.1 | direct bond to D | 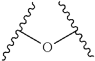 [SMILES: *O*] |
| 7.2 | 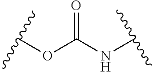 [SMILES: *NC(=O)O*] | 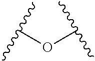 [SMILES: *O*] |
| 7.3 | 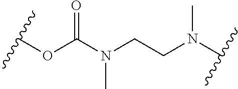 [SMILES: *OC(=O)N(C)CCN(*)C] | 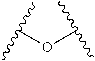 [SMILES: *O*] |
| 7.4 | 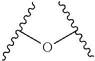 [SMILES: *O*] | 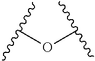 [SMILES: *O*] |
| 7.5 | 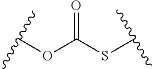 [SMILES: *OC(=O)S*] | 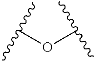 [SMILES: *O*] |
| 7.6 | 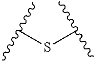 [SMILES: *S*] | 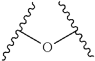 [SMILES: *O*] |

In one or more of the above embodiments L is

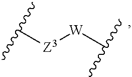

wherein:
W is a cleavable or non-cleavable peptide sequence selected from the group consisting of: Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gln, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gln, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and wherein Z³ is a bond or a spacer comprising a C₁₋₂₀ alkyl chain or a 1 to 10 polyethylene glycol (PEG)₁₋₁₀ or a C₂-amide-PEG₁₋₁₀.

Optionally L is

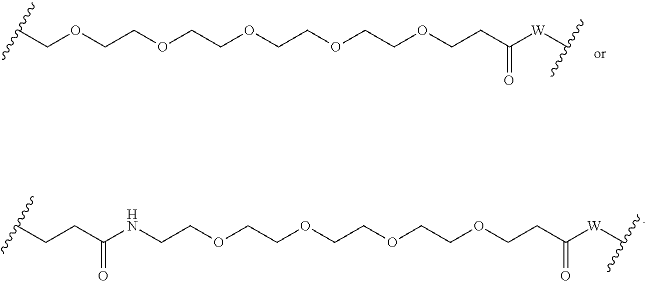

The cleavable peptide sequence W of L above is a target for a lysosomal protease, such that a TBDC internalized after recognizing and binding to the target sequence or target antigen and comprising the modular linker compound of the present disclosure becomes a substrate for a lysosomal protease. In human lysosomes, the predominant proteases are cysteine proteases of the cathepsin family, including cathepsin B, C, D, H, L, S and Z. Other relevant human lysosomal proteases involved in lysosomal protein degradation include: Legumain (also known as asparaginyl endopeptidase (AEP)), which is an asparagine endopeptidase; Pepstatin-insensitive protease 1, Pepstatin-sensitive protease A and Pepstatin-insensitive protease B, all of which are aspartic proteases. Optionally, the peptide sequence W is cleavable by cathepsin B. Once the peptide sequence W of L is cleaved, the electron cascade of the self-immolation reaction is triggered, leading to the traceless release of the drug substance from the self-immolative spacer X.

The cleavable peptide sequence W is the sequence targeted by the lysosomal proteases discussed above and may comprise non-proteinogenic amino acids such as citrulline (Cit) or modified amino acids such as the butyl ester of cysteine (cBu) unnatural, amino acid isomers such as isoaspartic acid-glutamic acid (iGlu) or N-acetyllysine (AcLys). As described, once cleaved, the electron cascade that leads to the elimination of the self-immolative spacer and the traceless release of the drug substance D is initiated. In a preferred embodiment, the dipeptide valine-alanine (Val-Ala) constitutes W. In an alternative preferred embodiment, the dipeptide valine-citrulline (Val-Cit) constitutes W. In yet another preferred embodiment, the dipeptide phenylalanine-lysine (Phe-Lys) constitutes W. In yet another preferred embodiment, W comprises the tripeptide glutamic acid-valine-alanine (Glu-Val-Ala) or glutamic acid-valine-citrulline (Glu-Val-Cit).

In some embodiments, Z³ is a bond directly connecting L to the branching unit Y. In alternative embodiments, Z³ comprises: a C₁₋₂₀ alkyl chain, optionally a C₂₋₁₈ or a C₄₋₁₆ or a C₆₋₁₄ or a C₈₋₁₂ alkyl chain; or a 1 to 10 polyethylene glycol (PEG)₁₋₁₀, optionally a PEG₂₋₈ or a PEG₄₋₆ or a PEG₅; or a C₂-amide-PEG₁₋₁₀, optionally a C₂-amide-PEG₂₋₈ or a C₂-amide-PEG₂₋₆ or a C₂-amide-PEG₄.

As such, in one preferred embodiment, L is

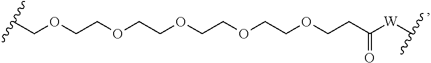

wherein W is Val-Ala.

In another preferred embodiment, L is

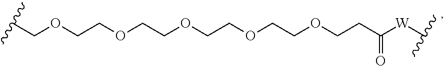

wherein W is Val-Cit.

In another preferred embodiment, L is

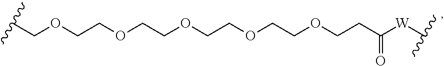

wherein W is Phe-Lys.

In another preferred embodiment, Lis

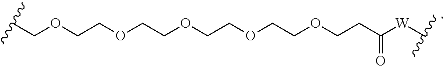

wherein W is Glu-Val-Ala.

In another preferred embodiment, L is

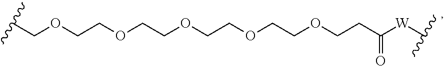

wherein W is Glu-Val-Cit.

In some embodiments, Y of the modular linker compound of the first aspect is:

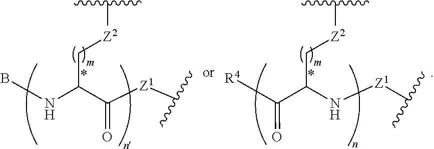

In such embodiments:
m is an integer from 1 to 6, optionally 1, 2, 3, 4, 5 or 6;
Z¹ connects Y to A and is

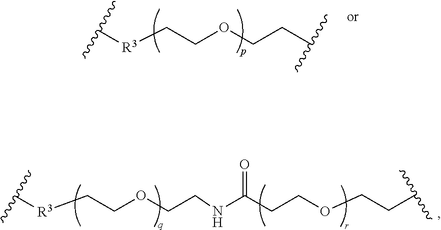

wherein
R³ is —O—, —NH—, —C(═O)—, —C(═O)N(CH₃)—, —C(═O) NH—, —NHC(═O)—, or —N(CH₃) C(═O)—, and
p is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14 or 8 to 12,
q is an integer from 1 to 8, such as 2, 3, 4, 5, 6, 7 or 8,
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
Z² connects Y to L and selected from the group consisting of:

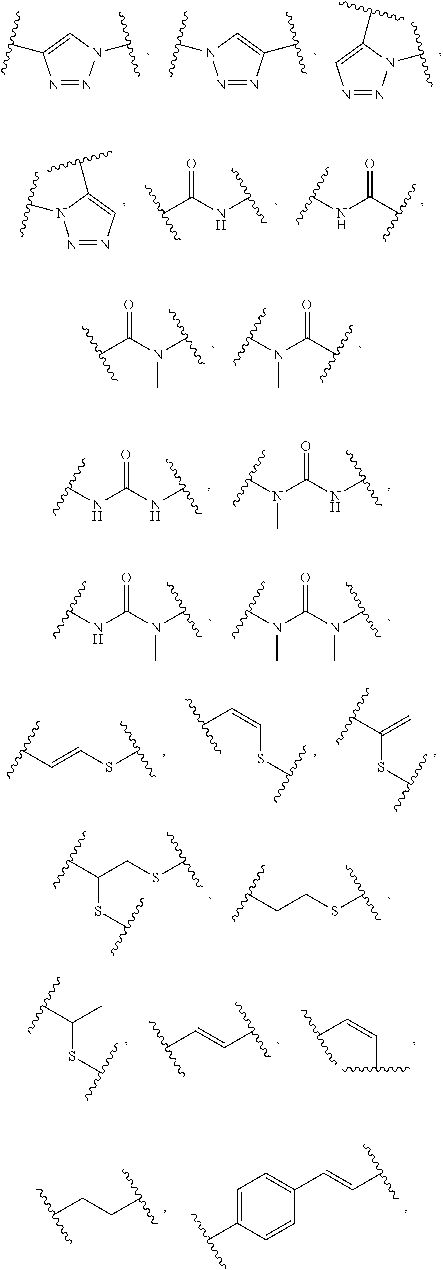

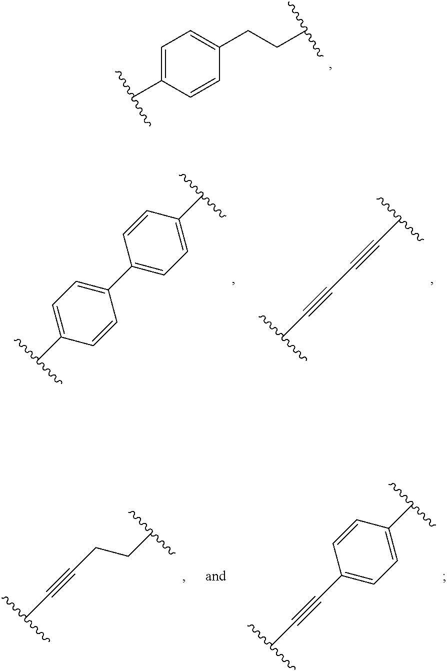 [SMILES: *CCc1ccc(*)cc1 ; *c1ccc(-c2ccc(*)cc2)cc1 ; *C#CC#C* ; *C#CCC* ; *C#Cc1ccc(*)cc1]

B is:
   a cap, optionally an acyl group, and n′ is n; or

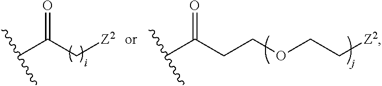

wherein
i is an integer from 1 to 20,
j is an integer from 1 to 10, and
n′ is n minus 1; or

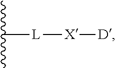

wherein
D′ is a pharmaceutically active substance different from D; and
X′ is self-immolative spacer

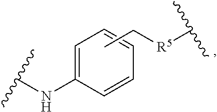

wherein:
R⁵ covalently links X′ to D′ in para or ortho to the amino group of X′ and is a bond,

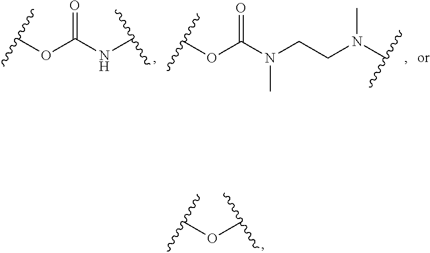

and n′ is n; and
R⁴ is OH or NH₂.

In a preferred embodiment Z² is a 1,2,3-triazole, an amide, or an urea derivative selected from the group consisting of

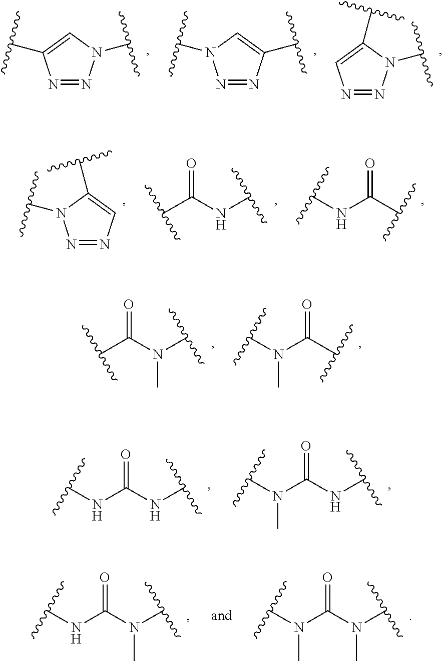

If Z² is selected from the group consisting of

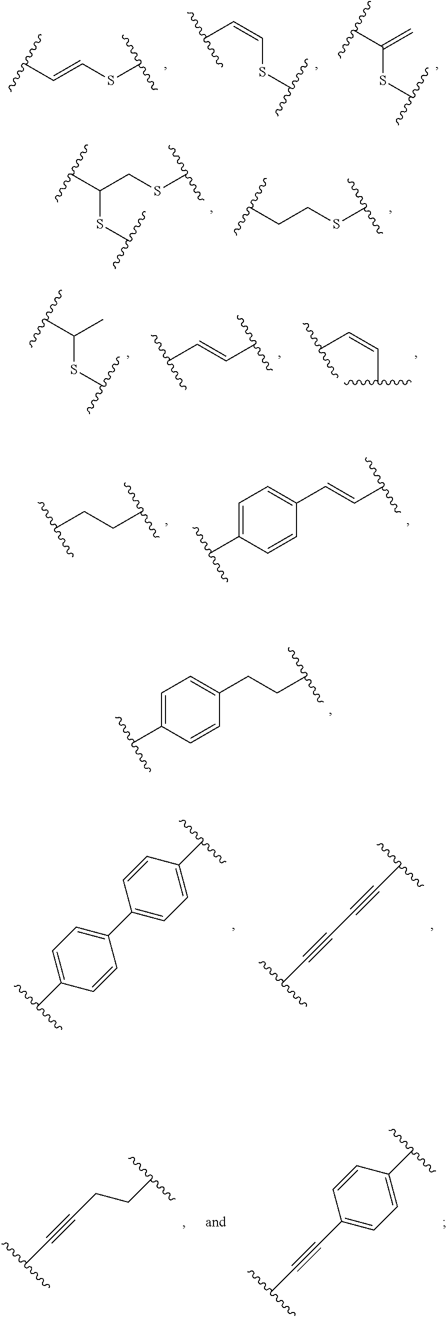

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In some embodiments, wherein B is

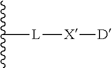

Z³ in L may further comprise a terminal carbonyl moiety, such as a dicarboxylic acid, for linkage of L to the amine group of Y.

Regardless of whether B is
a cap, optionally an acyl group, where n′ is n; or

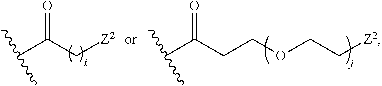

wherein
i is an integer from 1 to 20, such as 1, 2, 3, 4, 5, 6, 7, 8, 9, 10, 11, 12, 13, 14, 15, 16, 17, 18, 19 and 20,
j is an integer from 1 to 10, such as 1, 2, 3, 4, 5, 6, 7, 8, 9 and 10, and
n′ is n minus 1; or
a

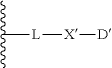

unit,
B constitutes the terminus of the final branching unit Y in the multimeric core of the modular linker compound in these embodiments.

As shown above, the multimeric core can comprise n′=2 to 5, optionally 2, 3, 4 or 5, branching units Y, wherein the branching unit proximal to A is connected to A via Z¹ and the final branching unit (i.e. the branching unit distal to A) comprises B. Each branching unit Y in this embodiment is indirectly linked to one molecule of a pharmaceutical drug substance D via spacer Z², spacer L and the immolative spacer X.

As such, the degree of loading (DOL) of the modular linker compound comprising a multimeric core comprising n′=5 branching units Y is either 5, if B is a cap structure, or 6, if B is

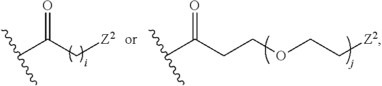

wherein
i is an integer from 1 to 20,
j is an integer from 1 to 10, and
n′ is n minus 1; or
a

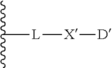

unit.

Consequently: when n′=4, the DOL is either 4 or 5, respectively; when n′=3, the DOL is either 3 or 4, respectively; and when n′=2, the DOL is either 2 or 3, respectively. Optionally, n′=2, B is a terminal acyl group, such as an acetyl group, as a cap and the DOL of the modular linker compound is 2.

In a particularly preferred embodiment, the branching unit Y is

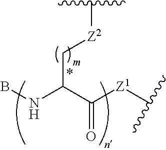

wherein:
B is an acyl cap, optionally an acetyl group,
n′=2,
Z² is the 1, 2, 3 triazole

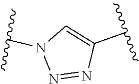 [SMILES: *c1cn(*)nn1]

and m=4, and
Z¹ is

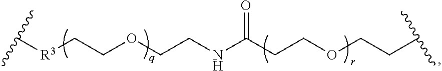

wherein
R³ is —NH—, q=3 and r=4.

In alternative embodiments, branching unit Y of the modular linker compound of the first aspect is:

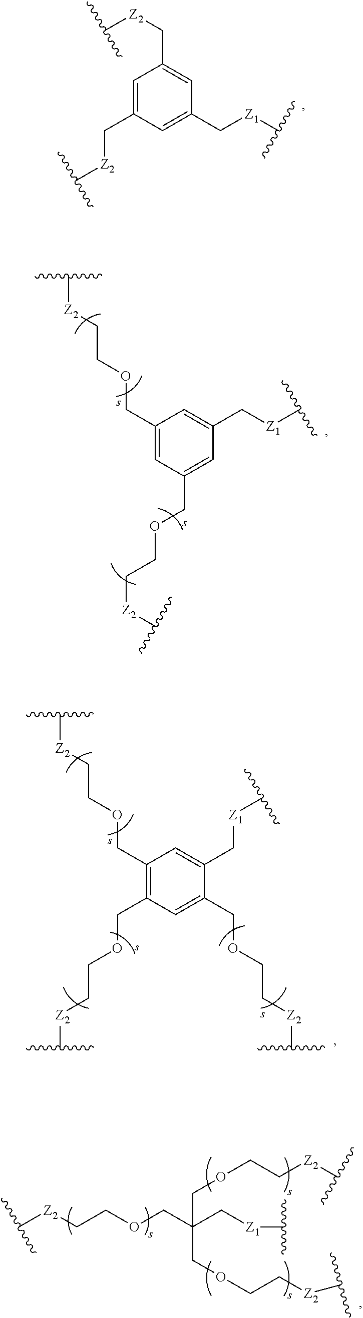

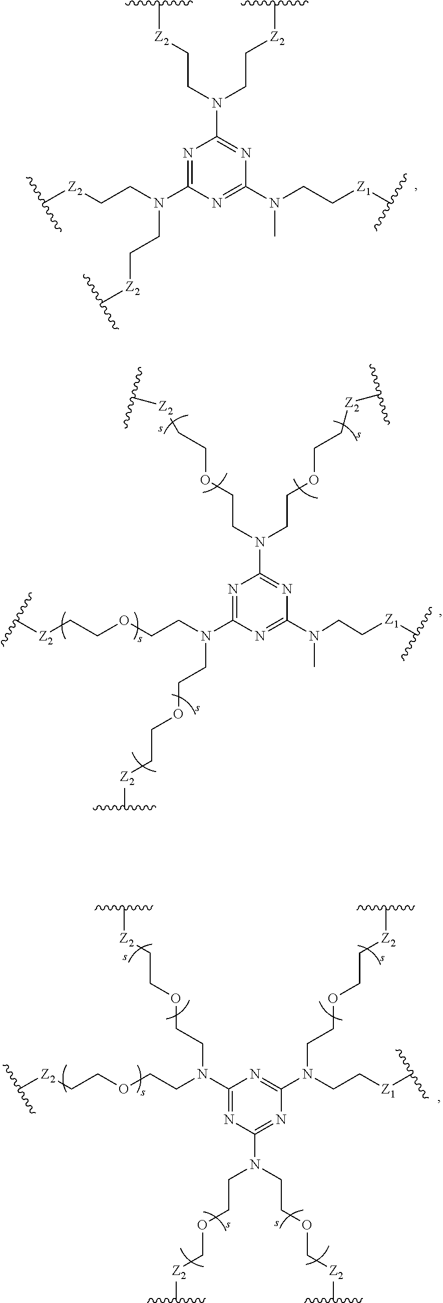

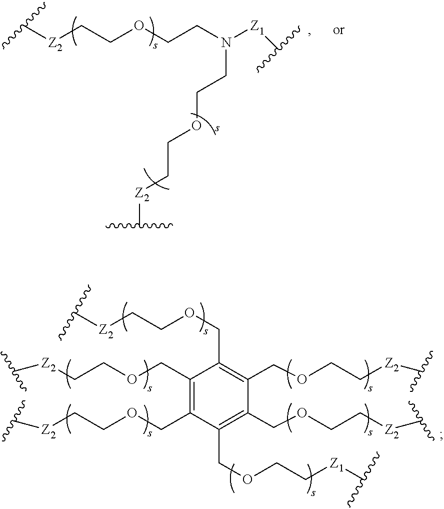

Z¹ connects Y to A and is

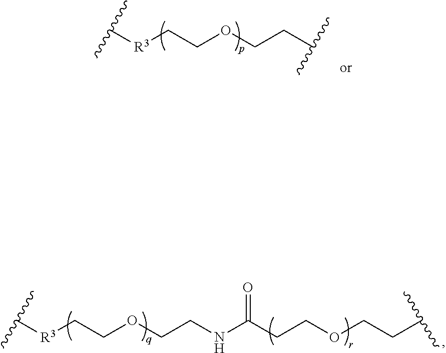

wherein
R³ is —O—, —NH—, —C(═O)—, —C(═O)N(CH₃)—, —C(═O) NH—, —NHC(═O)—, or —N(CH₃) C(═O)—,
p is an integer from 1 to 24, optionally from 4 to 24, optionally from 8 to 24 such as 8, 9, 10, 11, 12, 13, 14, 15, 16, 17, 18, 19, 20, 21, 22, 23 or 24;
q is an integer from 1 to 8, such as 1, 2, 3, 4, 5, 6, 7 or 8;
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
s is an integer from 0 to 4, such as 0, 1, 2, 3, or 4;
Z² connects Y to L and is selected from the group consisting of:

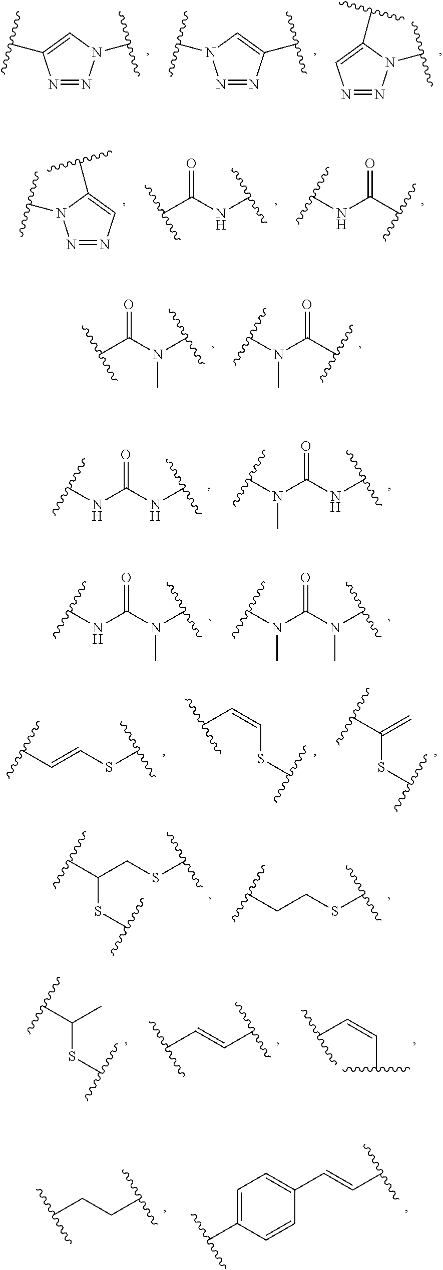

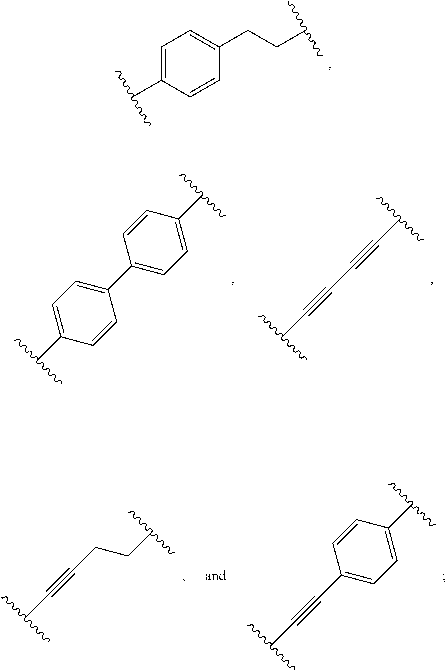 [SMILES: *CCc1ccc(*)cc1 ; *c1ccc(-c2ccc(*)cc2)cc1 ; *C#CC#C* ; *C#CCC* ; *C#Cc1ccc(*)cc1]

In a preferred embodiment Z² connects Y to L and is a 1,2,3-triazole, an amide, an amide, or an urea derivative, selected from the group consisting of:

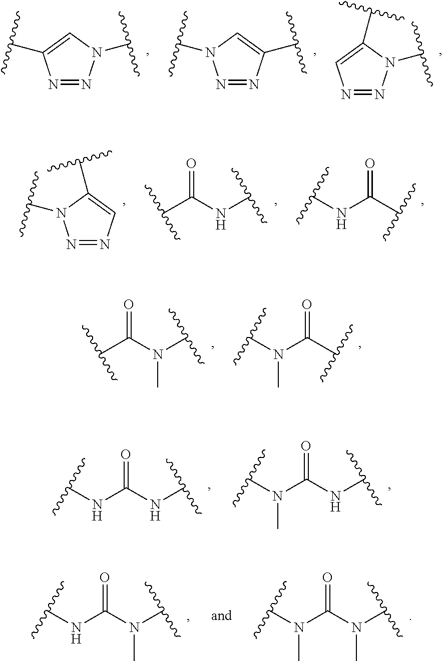

If Z² is selected from the group consisting

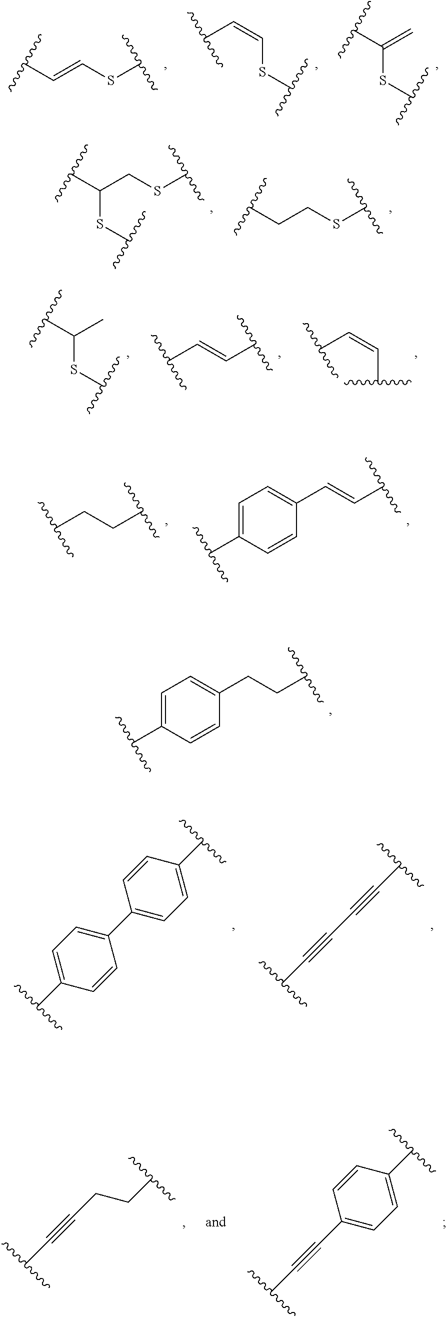

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

Depending on which of the alternative branching units Y shown above are employed, the DOL of the modular linker compound of the present disclosure can be 2, 3, 4, or 5.

Advantageously, the modular linker compounds of the present disclosure allow for variability with respect to the cytotoxic drug substance of the TBDC such that burying mechanisms of action may be used depending on the type of cancerous target cells and that the therapeutic outcome desired. Generally, the pharmaceutically active substances D and D′ are sufficiently potent to kill or inhibit the growth of the cancerous target cells at relatively low concentrations (e.g. at or below about 1 μM, 100 nM, 10 nM, 1 nM, 100 pM, 10 pM, 1 pM), while having a low immunogenicity profile, such as to ensure patient tolerance. Importantly, they are typically stable in systemic circulation and remain attached to the linker compound.

Accordingly, in some embodiments of the modular linker compound of the present disclosure, the pharmaceutically active substances D and D′, i.e. the cytotoxic drug substance or substances incorporated into the modular linker compound with the DOL described above and to be delivered to the cancerous target cells, are independently selected from the group consisting of: camptothecins, optionally exatecan, SN38, and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; toll-like receptor 7 (TLR7) agonists; nicotinamide phosphoribosyltransferase (NAMPT) inhibitor (NAMPTᵢ): pyrrolobenzodiazepines (PBDs); duocarmycins; anthracyclines, optionally doxorubicin; amatoxins, optionally alpha-amanitin, beta-amanitin, amanin and amaninamide; cryptophycins; phalloidins; seco-cyclopropylpyrroloindoline (seco-CPI); seco-cyclopropylbenzoindoline (seco-CBI); taxols; vinblastines; HDAC inhibitors; colchicines; eribulins; methotrexate (MTX); triptolide; and derivatives thereof.

As also mentioned above, many of these substances may nevertheless have an unfavorably low solubility in aqueous solutions such as physiological/systemic solutions. Therefore, in order to avoid undesirable aggregation of TBDCs comprising the modular linker compounds of the present disclosure, the modular linker compound necessarily comprises solubility enhancing group SE. Solubility enhancing group SE may be an alpha-cyclodextrin, a beta-cyclodextrin, a gamma-cyclodextrin, a polysarcosine, a PEG, a galactoside, a glucuronide, or a polyalcohol made from carbohydrates.

Cyclodextrins are cyclic oligosaccharides composed of glucose units arranged in a toroid (doughnut) shape. They have a hydrophobic interior and a hydrophilic exterior. This unique structure allows them to form inclusion complexes with hydrophobic or poorly soluble molecules and when a hydrophobic/lipophilic pharmaceutical drug substance is complexed with a cyclodextrin, its solubility in an aqueous solution is typically increased. Accordingly, a hydrophobic pharmaceutically active drug substance can be encapsulated within the hydrophobic pocket of the cyclodextrin, shielding it from the surrounding aqueous environment thereby enhancing its solubility. Notwithstanding, cyclodextrins are chemically compatible with a wide range of pharmaceutically active drug substances and typically are non-reactive with respect to molecules encapsulated via the formation of an inclusion complex. This allows for the maintenance of complex stability and integrity of the pharmaceutically active drug substance at the same time.

Further, cyclodextrins have been widely used in pharmaceuticals, food as well as cosmetics for many years and it is established that their overall safety and biocompatibility profiles are favourable.

Accordingly, cyclodextrins are particularly suitable solubility enhancing groups SE in the context of the here-described linker compounds. For example, alpha-cyclodextrins (comprising six glucose molecules), beta-cyclodextrins (comprising seven glucose molecules) and gamma-cyclodextrins (comprising eight glucose molecules), when attached to a PEG chain either directly or via a spacer Z⁴ such as:

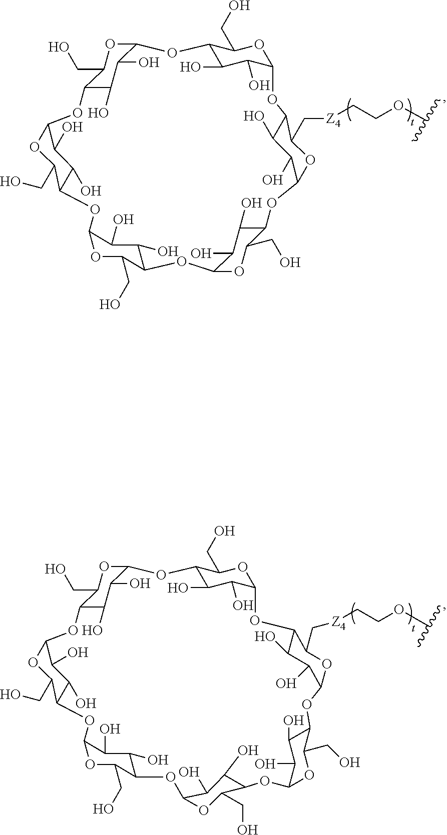

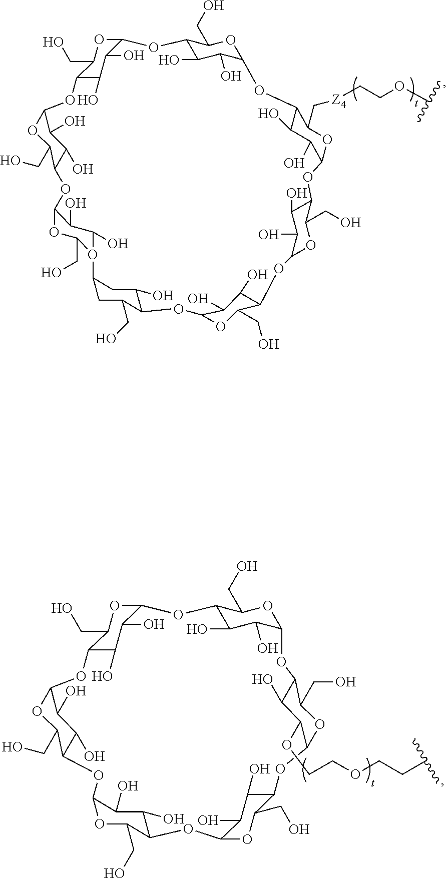

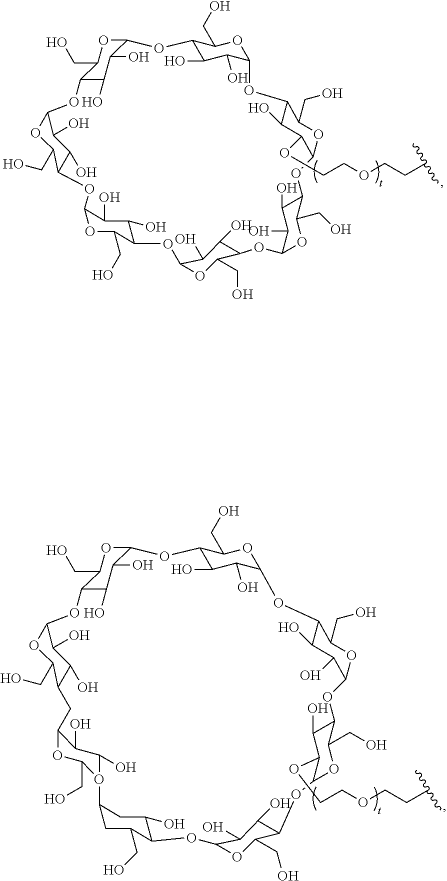

wherein Z⁴ is

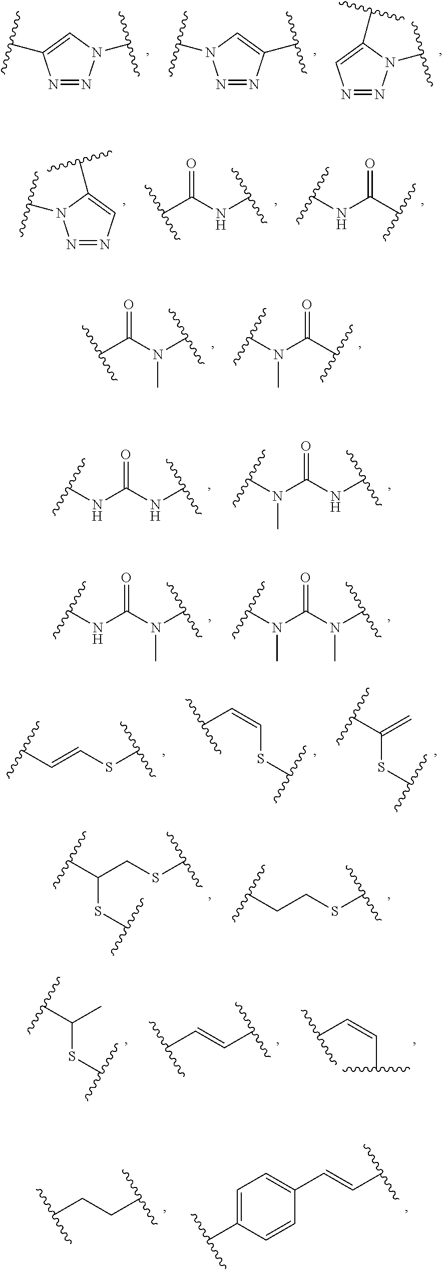

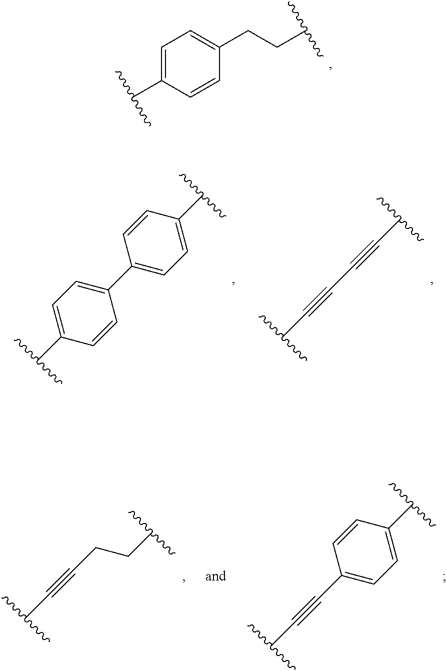 [SMILES: *CCc1ccc(*)cc1 ; *c1ccc(-c2ccc(*)cc2)cc1 ; *C#CC#C* ; *C#CCC* ; *C#Cc1ccc(*)cc1]

and wherein the PEG chain comprises between 2 and 19 PEGs, i.e, wherein t is 2 to 18, 4 to 16, 6 to 14 and most optionally 8, 9, 10, 11 or 12,
constitute suitable variance of the solubility enhancing group SE.

In a preferred embodiment Z⁴ is a 1,2,3-triazole, an amide, an amide, or an urea derivative, selected from the group consisting of:

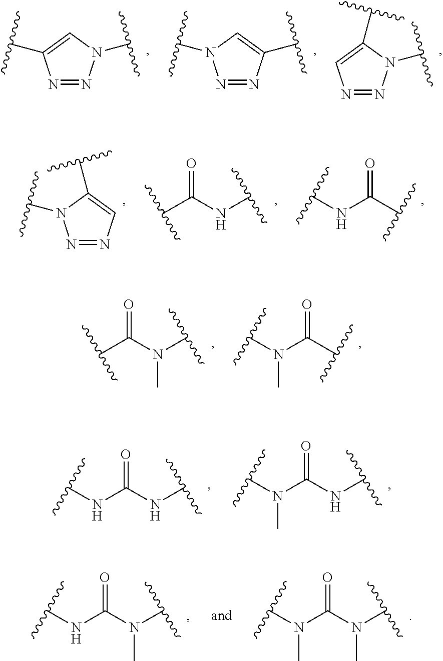

If Z⁴ is selected from the group consisting of

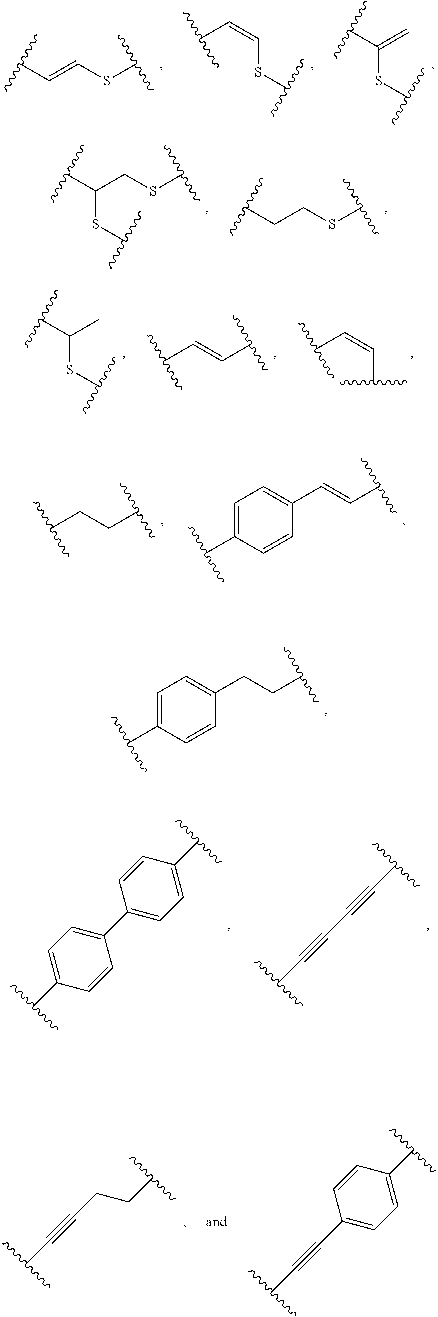

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In an embodiment where the pharmaceutical drug substance D within a

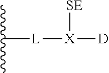

unit is a camptothecin, it is particularly advantageous to link a cyclodextrin enhancing group of the above illustrated types to the self-immolative spacer X via an 8 to 12 membered PEG chain, i.e. where t=8 to 12 (e.g. 8, 9, 10, 11 or 12). Such PEG chain lengths favour a conformational arrangement of the linker compound that allows for an inclusion complex placing the lipophilic drug substance into the pocket of the cyclodextrin bearing structure substantially negating the hydrophobic/lipophilic properties of the camptothecin.

Other suitable solubility enhancing groups include other polar groups such as
a polysarcosine

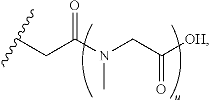

wherein u is at least 1, optionally 2, 3, 4, 5, 6, 7, 8, 9, 10, 12 or 14;
a polysarcosine amide

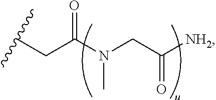

wherein u is at least 1, optionally 2, 3, 4, 5, 6, 7, 8, 9, 10, 12 or 14;
a PEG chain

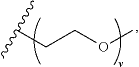

wherein v is 4 to 24 such as 4 to 22, 4 to 20, 4 to 18, 4 to 16, 4 to 14, 4 to 12, 4 to 10, or 4, 5, 6, 7, 8, 9, 10, 11 or 12;
a

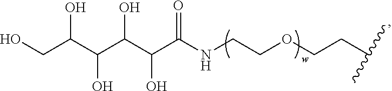

a PEG-glucamide

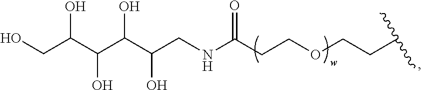

a

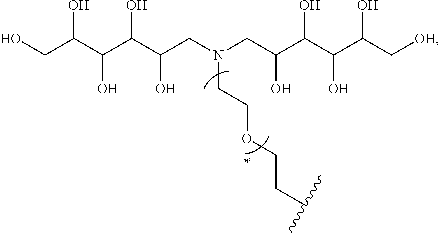

a

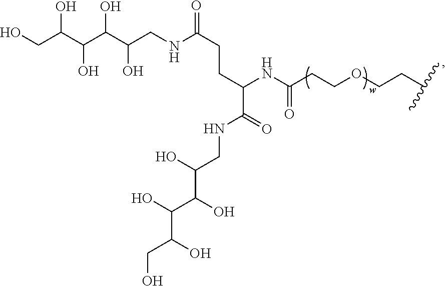

a

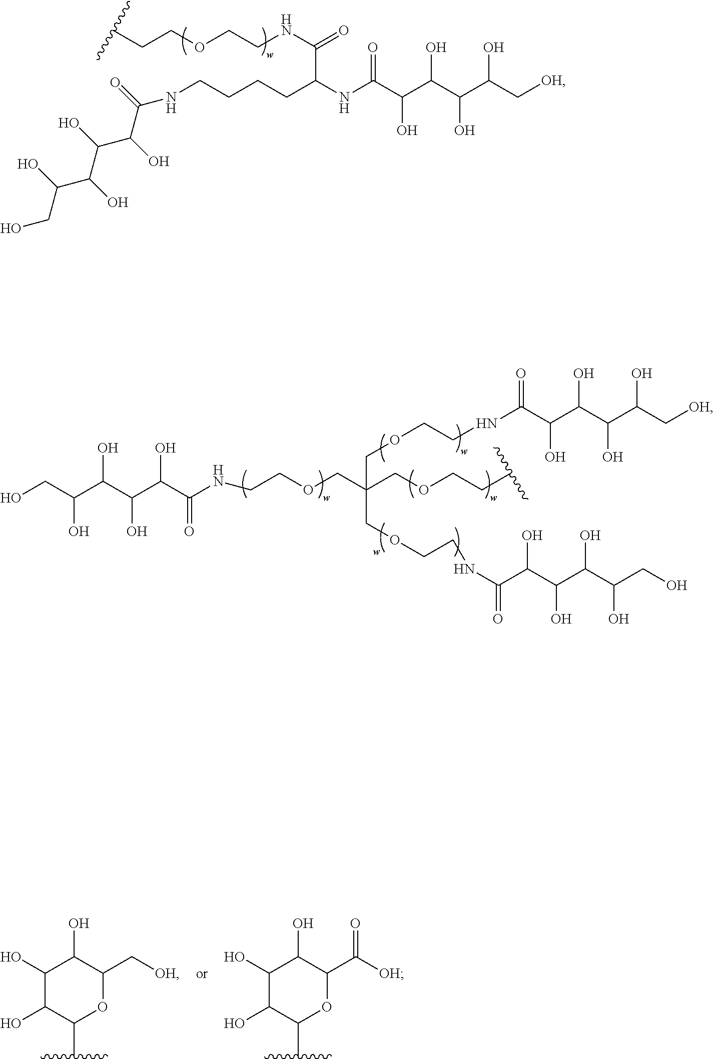

wherein w is 2 to 24 such as 2 to 22, 2 to 20, 2 to 18, 2 to 16, 2 to 14, 2 to 12, 2 to 10, or 2, 3, 4, 5, 6, 7, 8, 9, 10, 11 or 12.

The terminal reactive group A of a modular linker compound of the present disclosure ensures specific and controlled conjugation of the linker compound to the target-binding moiety such that a stable TBDC can be generated. In this regard, various reactive groups A have proven useful. For example, in some embodiments, the reactive group A is a thiol-reactive group such as
a 3-maleimidopropanamide group

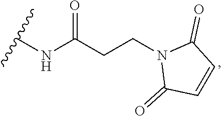 [SMILES: *NC(=O)CCN1C(=O)C=CC1=O]

a 6-maleimidohexanamide group or

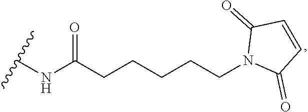 [SMILES: *NC(=O)CCCCCN1C(=O)C=CC1=O]

a 3-amino-2-maleimidopropanamide group

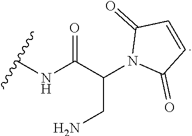 [SMILES: *NC(=O)C(CN)N1C(=O)C=CC1=O]

Thiol reactive groups have been shown to provide robust and versatile means for conjugation of linker compounds to TBDCs. They allow for site-specific conjugation of via correspondingly reactive thiol groups in the target-binding moiety. Specifically, they allow for conjugation of the modular linker compound of the present disclosure to a naturally occurring sulfhydryl moiety in the target-binding moiety or to a sulfhydryl moiety, which has been introduced into the target-binding moiety by genetic engineering as described in e.g. Junutula Nat Biotechnol. 2008 August; 26 (8): 925-32. Optionally, the modular linker compound disclosed herein is conjugated via one of the above-shown thiol-reactive groups to sulfhydryl moieties that have been introduced into the Fc region of target binding moiety to which the linker is conjugated by genetic engineering as disclosed herein above, for example in an antibody comprising a D265C (according to EU numbering, Edelman et al., Proc. Natl. Acad. Sci. USA; 63 (1969) 78-85) substitution.

Beneficially, thiol reactions are highly efficient and typically proceed rapidly under mild conditions. This means that thiol-reactive groups can form conjugates quickly and with a high degree of completeness. Further, the thioether bonds formed between thiol groups and thiol-reactive groups are stable under physiological conditions. While they are less prone to hydrolysis or degradation compared to some other types of chemical bonds, further stability of the conjugate can be achieved through incorporation of a basic amino group adjacent to the maleimide as described in Lyon et al. Nat. Biotechnol. 2014, 32, 1059-1062.

Further, thiol-reactive chemistry is highly flexible, offering variety of thiol-reactive groups to be incorporated into the modular linker compound, is generally biocompatible (i.e. does not introduce highly toxic or immunogenic components into a TBDC), and allows for precise control over the drug-to-antibody ratio (DAR) in a TBDC, which is key in optimizing therapeutic efficacy and minimizing off-target effects as described above.

In alternative embodiments, reactive group A can be a 3-(2-((1,2-dimethylhydrazineyl)methyl)-1H-indol-1-yl) propanamide or 7-aza-derivative for conjugation to formylgycine moiety in a target binding moiety via the Hydrazino-Pictet-Spengler (HIPS) Ligation

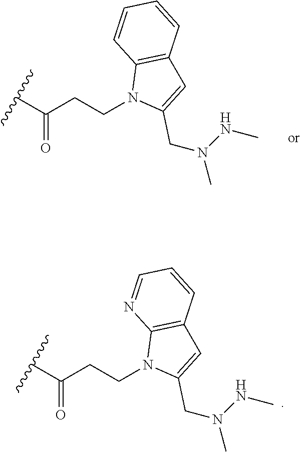 [SMILES: *C(=O)CCn1c(CN(C)NC)cc2ccccc21 ; *C(=O)CCn1c(CN(C)NC)cc2cccnc21]

In alternative embodiments, reactive group A can be a compound selected from DBCO (azadibenzocyclooctyne) or BCN (a bicyclo[6.1.0]non-4-yn-9-ylcarbonyl) which are strained cycloalkynes, intended for conjugation to azide containing or modified antibodies e.g. via 4-azido-phenylalanine or azide modified glycan side chains, for example, “SiteClick”. In particular, the reactive group A can be

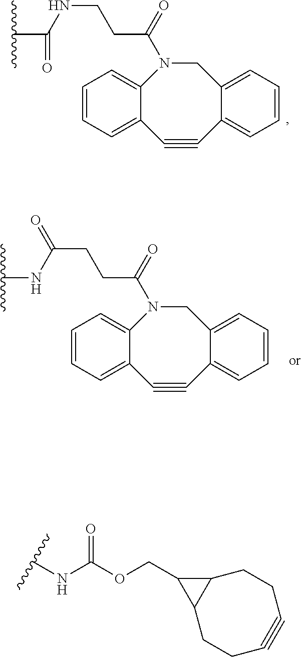 [SMILES: *C(=O)NCCC(=O)N1Cc2ccccc2C#Cc2ccccc21 ; *NC(=O)CCC(=O)N1Cc2ccccc2C#Cc2ccccc21 ; *NC(=O)OCC1C2CCC#CCCC21]

In further alternative embodiments, reactive group A can be a glycine derivative, which is particularly useful for enzymatic coupling reactions using Sortase A. Exemplary glycine derivatives may, be a polyglycine comprising between 2-5, e.g. 2, 3, 4 and 5, glycine residues:

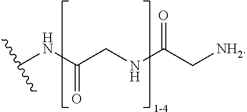

Glycine is a particularly small amino acid and its incorporation into the modular linker compound of the present disclosure and, ultimately into the TBDC to be prepared, minimizes steric hindrance while allowing for flexible attachment without disrupting the structure of either the linker compound or the target-binding moiety.

In further alternative embodiments, reactive group A can be a primary amine, e.g., a lysine group

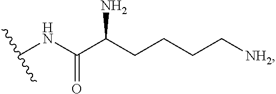 [SMILES: *NC(=O)[C@@H](N)CCCCN]

or any group comprising a primary amine (e.g. amino-modified PEG). Such conjugations may e.g. be done using transglutaminases as described in, for example, WO2015/162563 A1.

Using a lysine group allows for controlled conjugation of the modular linker compound to selected correspondingly reactive sites in the target-binding moiety (for example, an antibody modified to contain site specific maleimide or N-hydroxysuccinimide (NHS) ester adducts) via their reaction with the primary amine group (—NH₂) of the lysine.

The versatility of lysine residues as conjugation sites in target-binding moieties such as antibodies has made them valuable in ADC development because they have long been utilized for controlled (yet stochastic) attachment of drugs or other therapeutic molecules to antibodies. However, specifically modifying particular residues within a target-binding moieties backbone structure such as to present specific attachment points for lysine carrying linker compounds has proven useful in generating TBDCs with a specific DOL and DAR.

In preferred embodiments, the modular linker compounds of the present disclosure are selected from

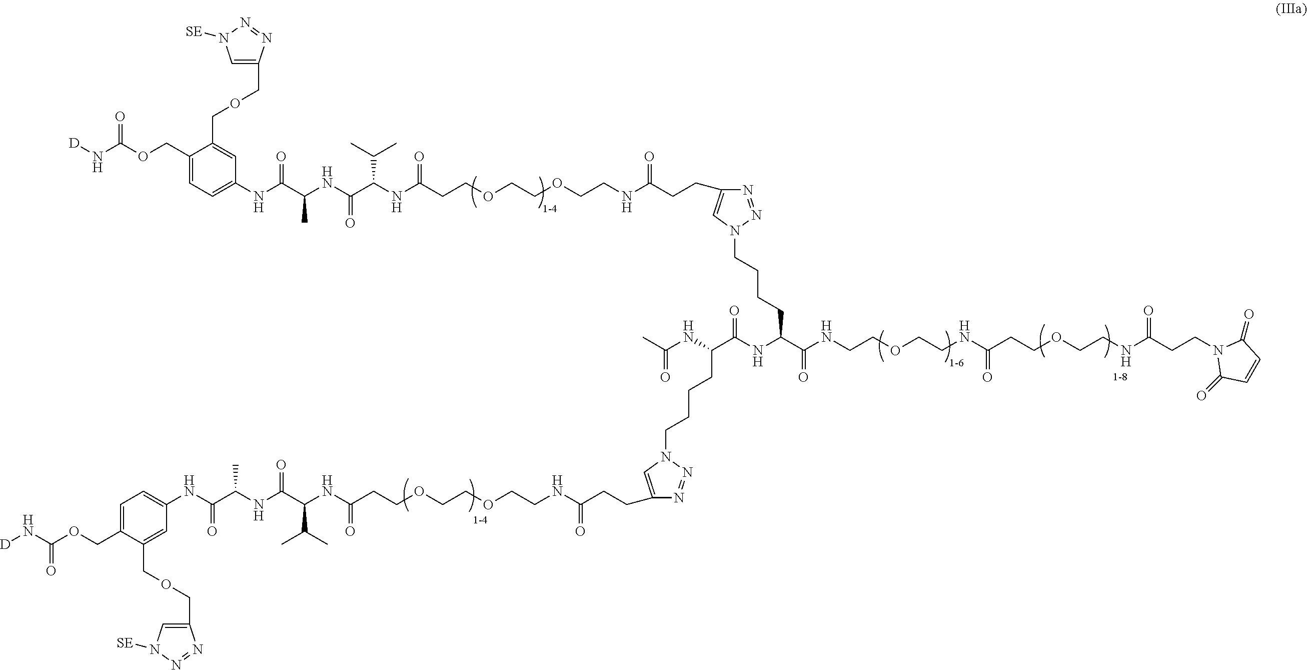

wherein,
SE is a solubility enhancing group as disclosed herein above,
D is pharmaceutically active substance as disclosed above, optionally D is selected from camptothecins, optionally exatecan, SN38, and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; toll-like receptor 7 (TLR7) agonists; nicotinamide phosphoribosyltransferase (NAMPT) inhibitor (NAMPT) i: duocarmycins; anthracyclines, optionally doxorubicin; amatoxins optionally alpha-amanitin, beta-amanitin, amanin and amaninamide; phalloidins; seco-cyclopropylpyrroloindoline (seco-CPI); seco-cyclopropylbenzoindoline (seco-CBI); taxols; vinblastines; colchicines; triptolide; and derivatives thereof.

In more preferred embodiments, the modular linker compounds of the present disclosure are selected from

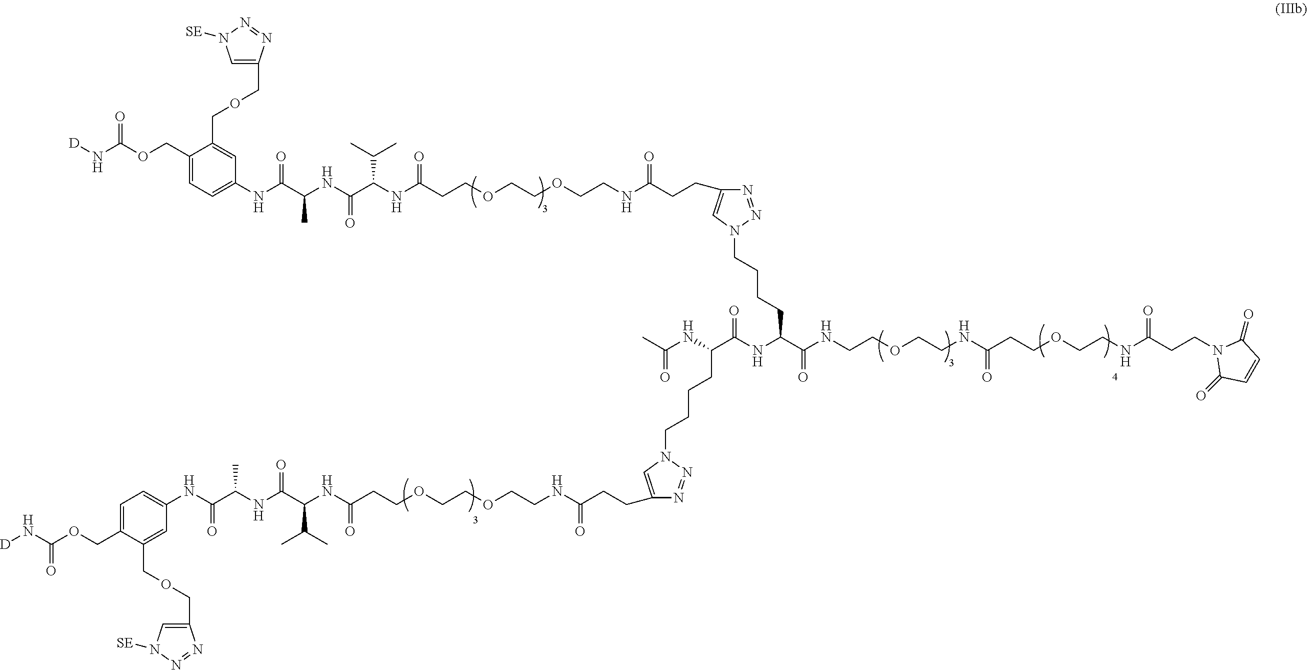

wherein
SE is a solubility enhancing group as disclosed herein above,
D is pharmaceutically active substance as disclosed above, optionally D is selected from camptothecins, optionally exatecan, SN38, and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; toll-like receptor 7 (TLR7) agonists; nicotinamide phosphoribosyltransferase (NAMPT) inhibitor (NAMPT) i: duocarmycins; anthracyclines, optionally doxorubicin; amatoxins optionally alpha-amanitin, beta-amanitin, amanin and amaninamide; phalloidins; seco-cyclopropylpyrroloindoline (seco-CPI); seco-cyclopropylbenzoindoline (seco-CBI); taxols; vinblastines; colchicines; triptolide; and derivatives thereof.

According to even more preferred embodiments, the modular linker compounds of the present disclosure are selected from the group comprising:

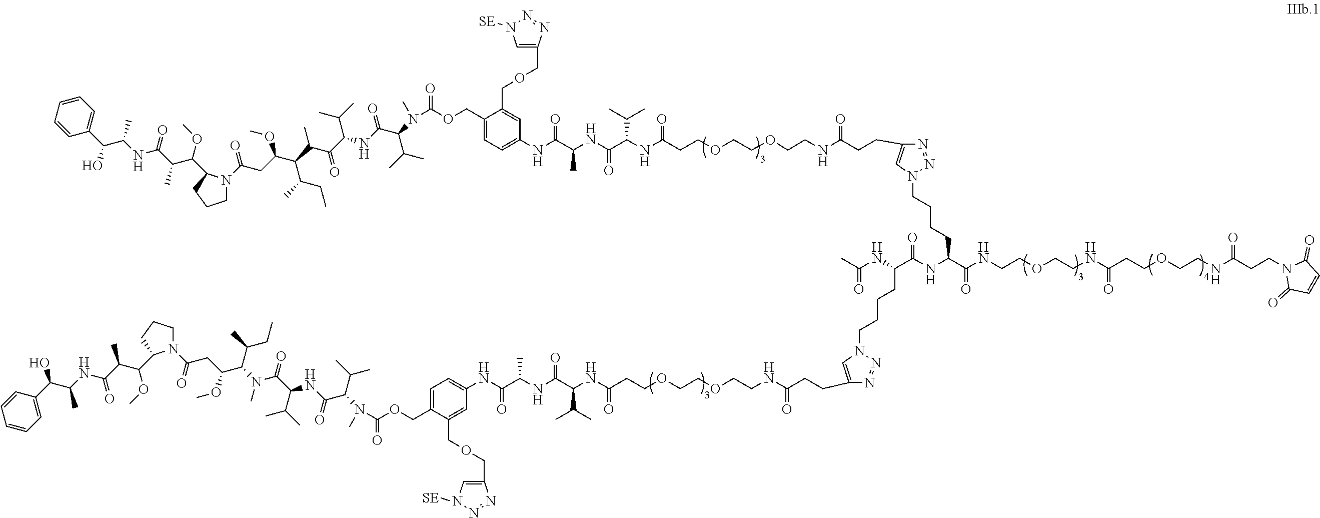

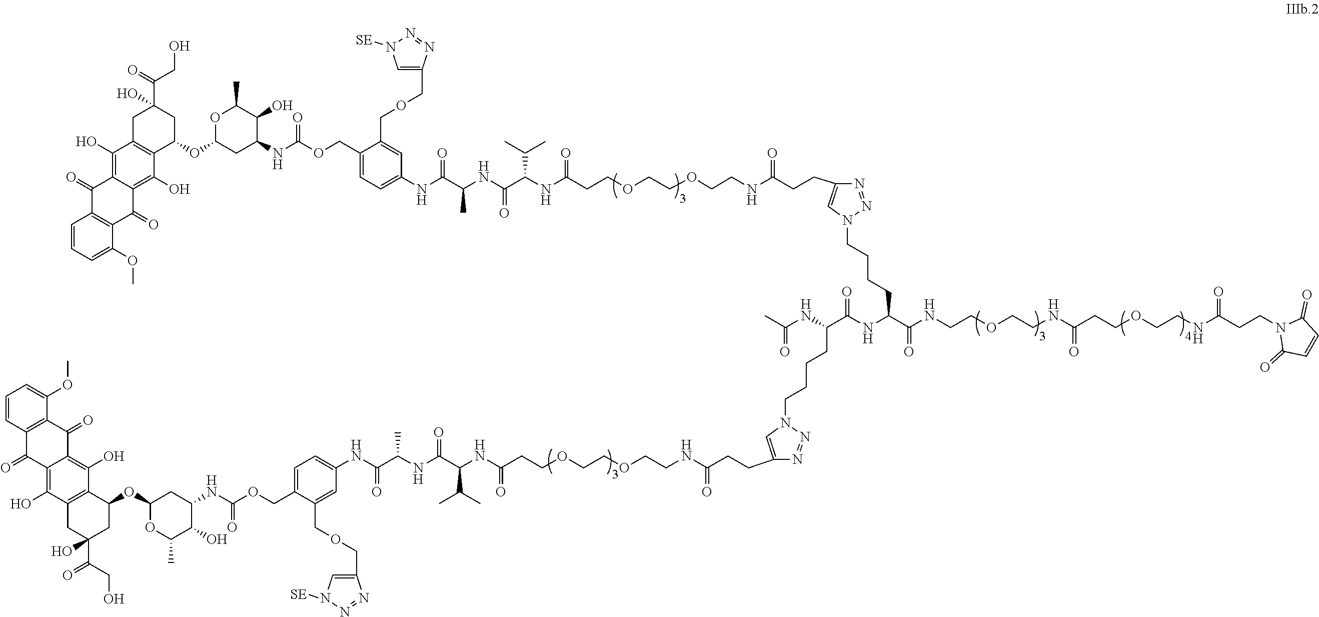

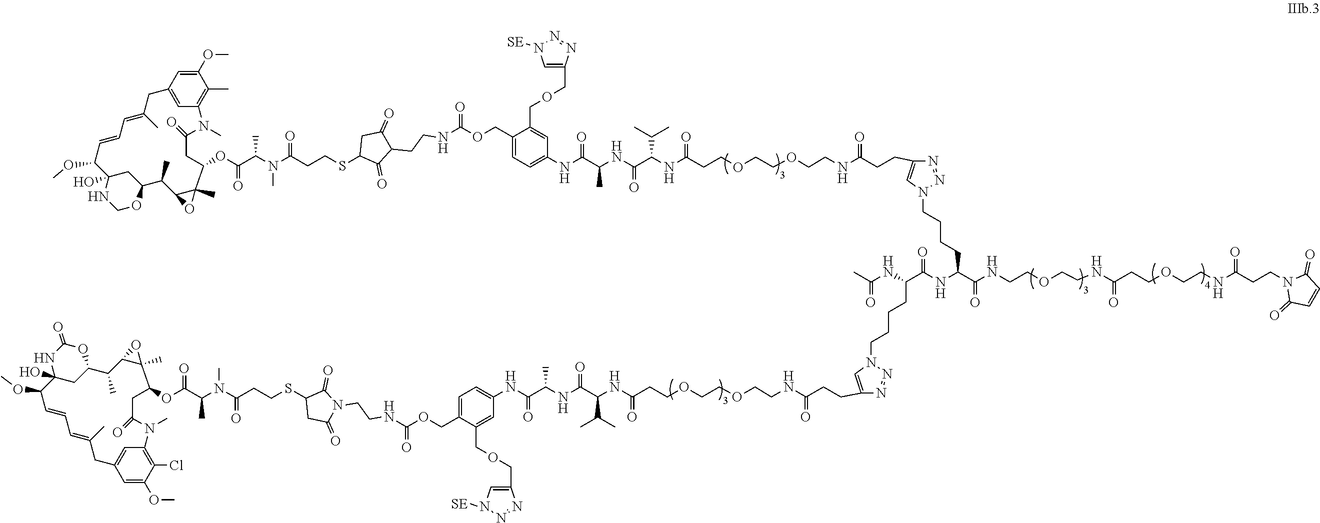

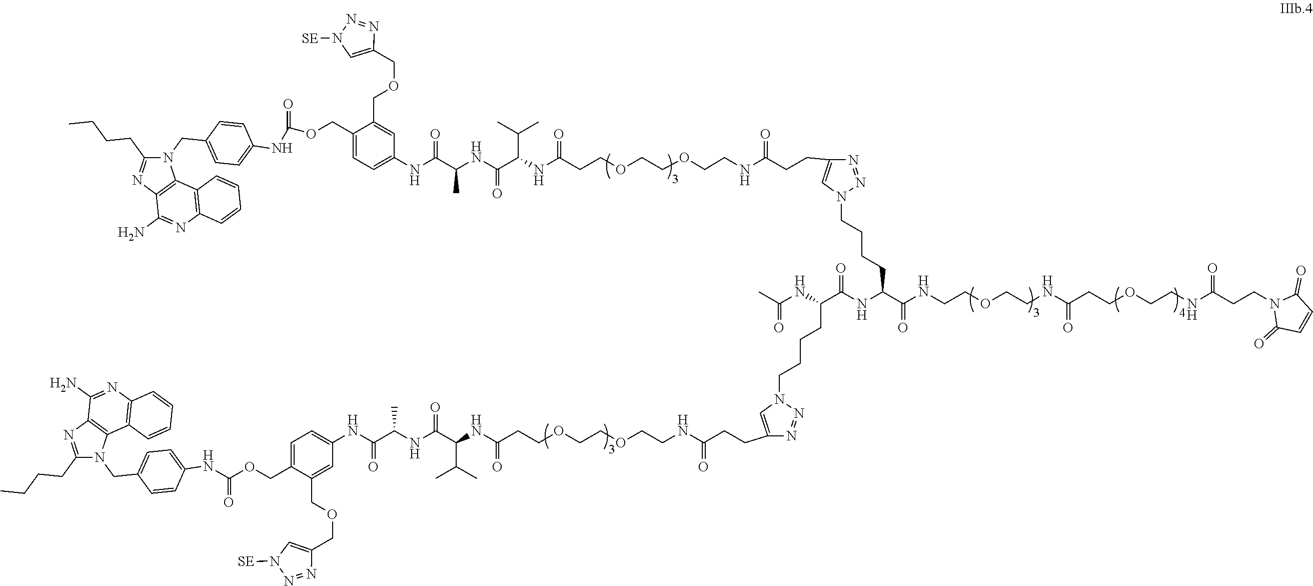

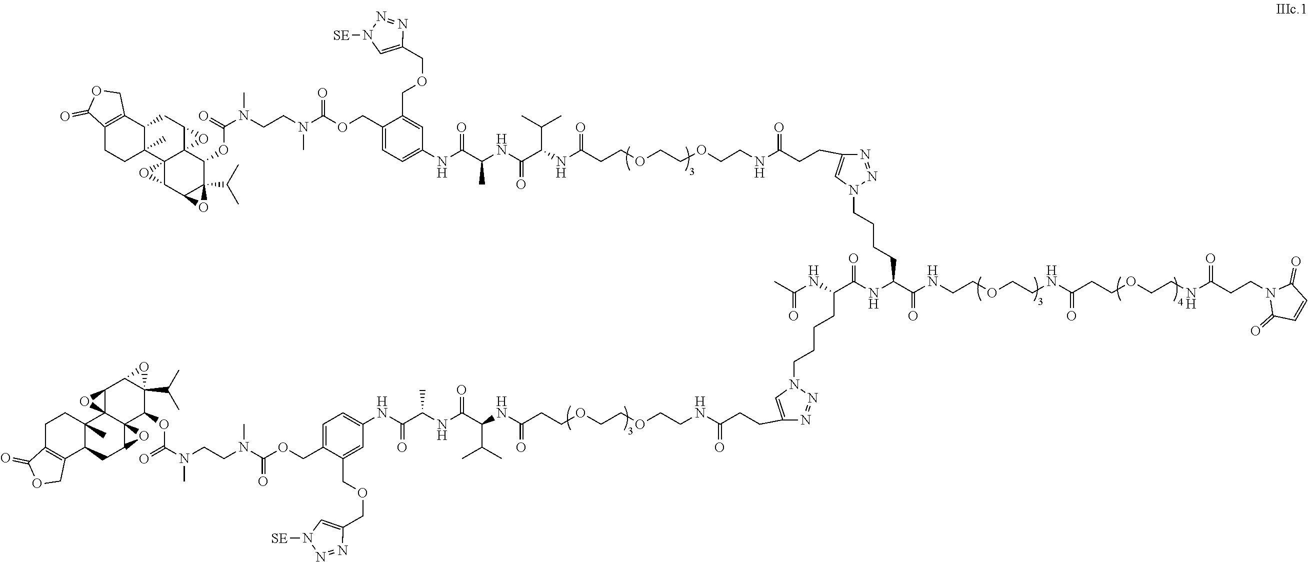

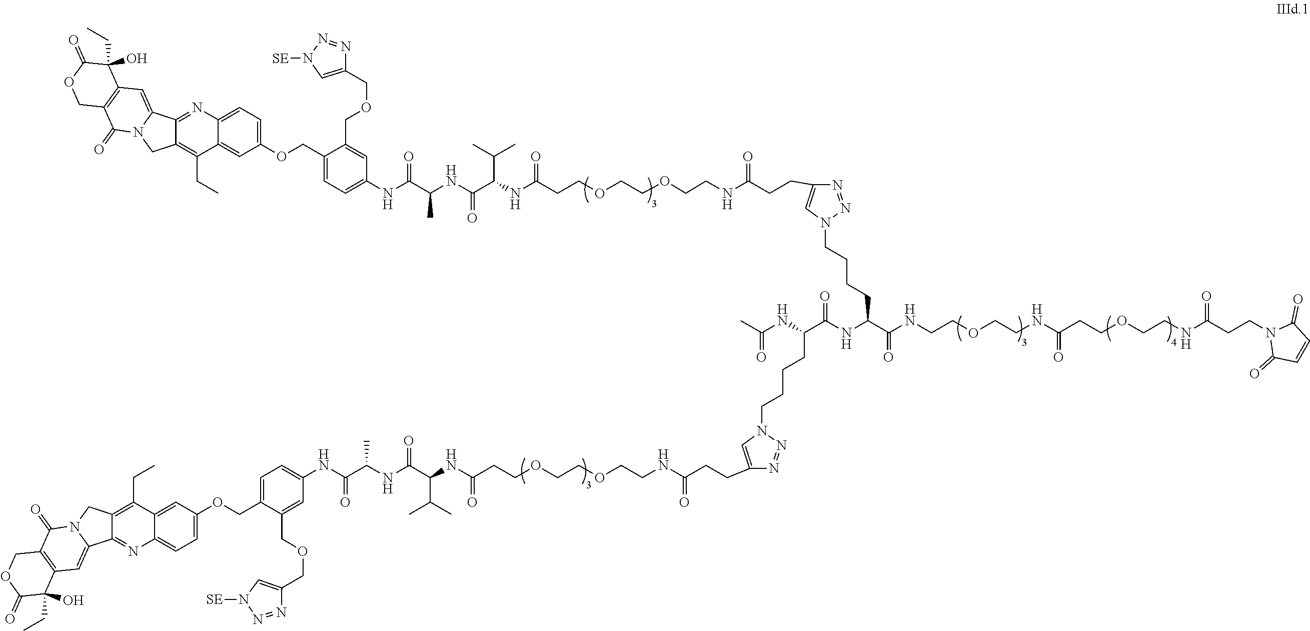

wherein SE is selected from
   a polysarcosine

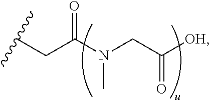

wherein u is at least 1, optionally 2, 3, 4, 5, 6, 7, 8, 9, 10; 12, or 14,
a polysarcosine amide

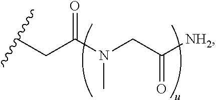

wherein u is at least 1, wherein u is at least 1, optionally 2, 3, 4, 5, 6, 7, 8, 9, 10; 12, or 14;
a PEG chain

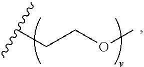

wherein v is 4 to 24 such as 4 to 22, 4 to 20, 4 to 18, 4 to 16, 4 to 14, 4 to 12, 4 to 10, or 4, 5, 6, 7, 8, 9, 10, 11, 12, or 14;
a

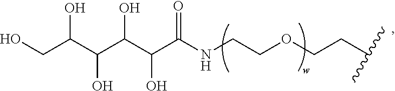

a PEG-glucamide

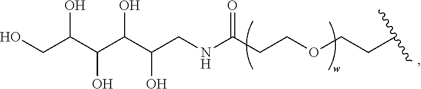

a,

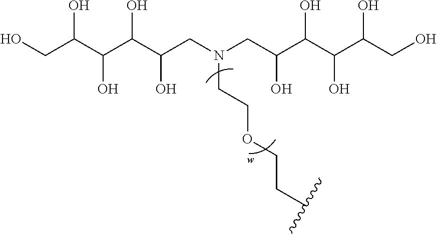

a

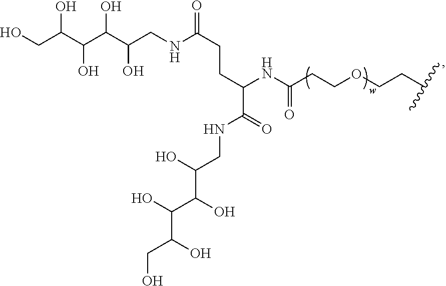

a

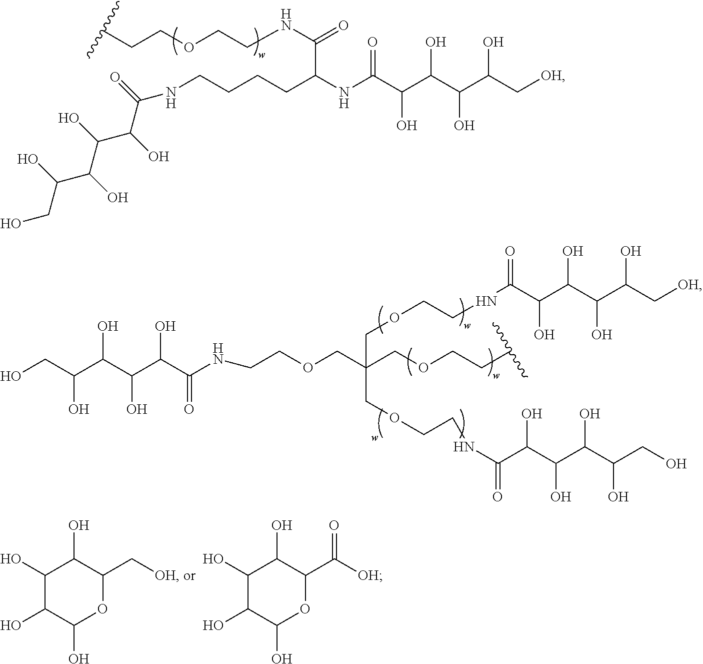

wherein w is 2 to 24 such as 2 to 22, 2 to 20, 2 to 18, 2 to 16, 2 to 14, 2 to 12, 2 to 10, or 2, 3, 4, 5, 6, 7, 8, 9, 10, 11 or 12, or
   alpha-cyclodextrin, beta-cyclodextrin or gamma-cyclodextrin, attached to a PEG chain either directly or via a spacer Z⁴ such as

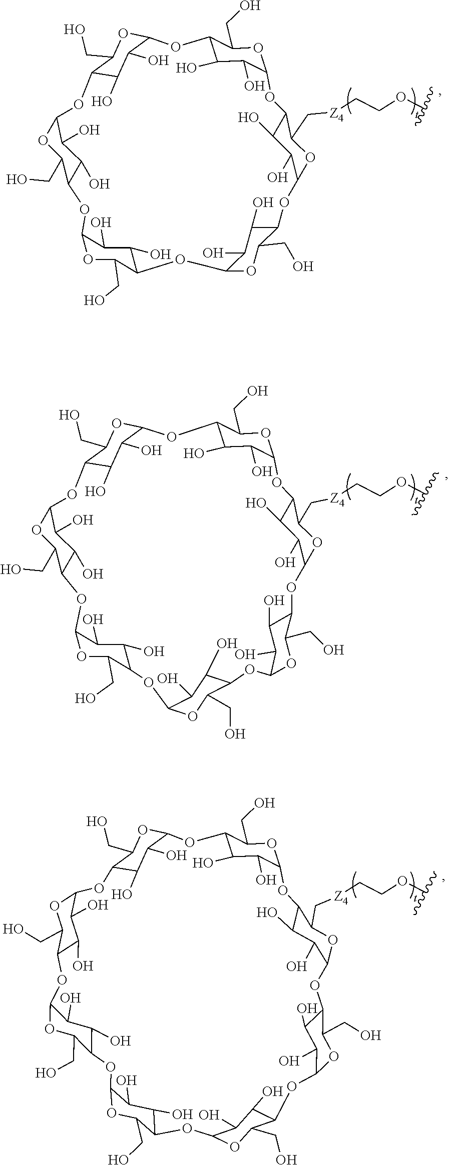

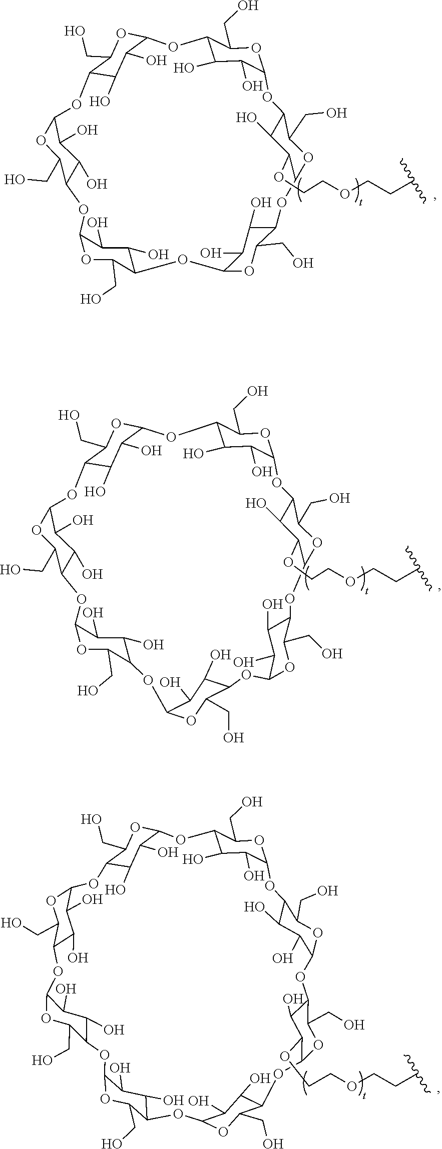

wherein Z⁴ is

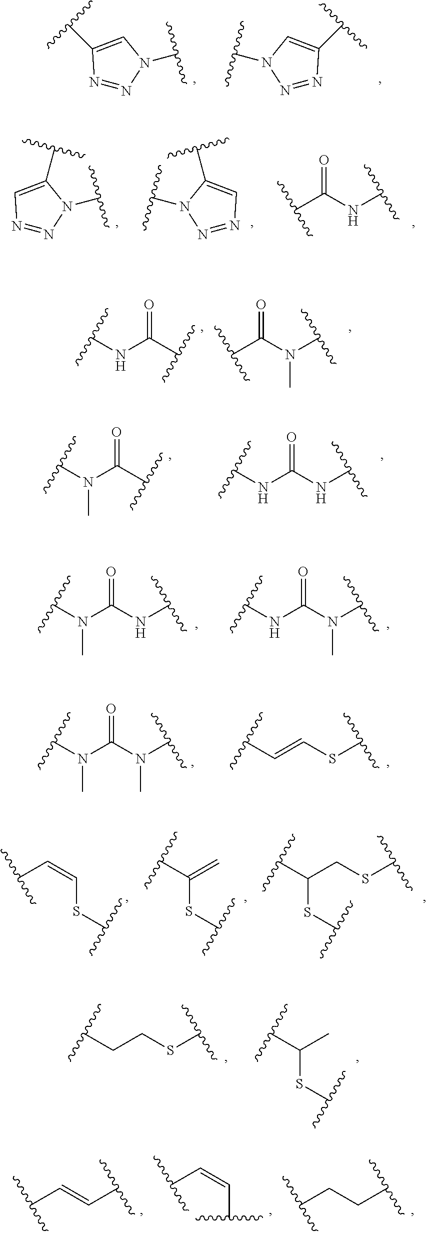

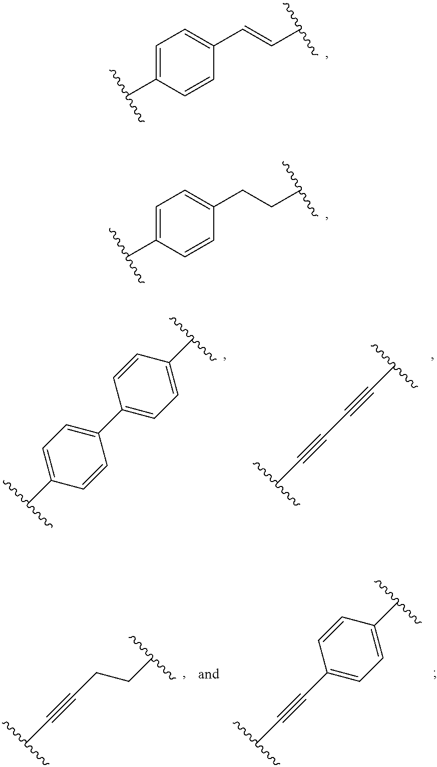 [SMILES: */C=C/c1ccc(*)cc1 ; *CCc1ccc(*)cc1 ; *c1ccc(-c2ccc(*)cc2)cc1 ; *C#CC#C* ; *C#CCC* ; *C#Cc1ccc(*)cc1]

and wherein the PEG chain comprises between 2 and 19 PEGs, i.e, wherein t is 2 to 18, 4 to 16, 6 to 14 and most optionally 8 to 12.

In a preferred embodiment Z⁴ is a 1,2,3-triazole, an amide, an amide, or an urea derivative, selected from the group consisting of:

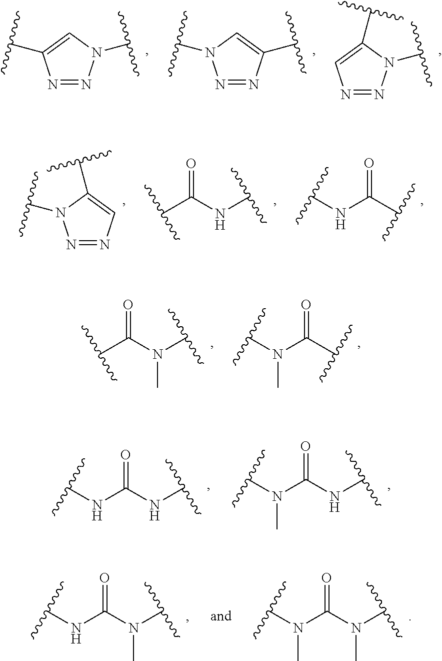

If Z⁴ is selected from the group consisting of

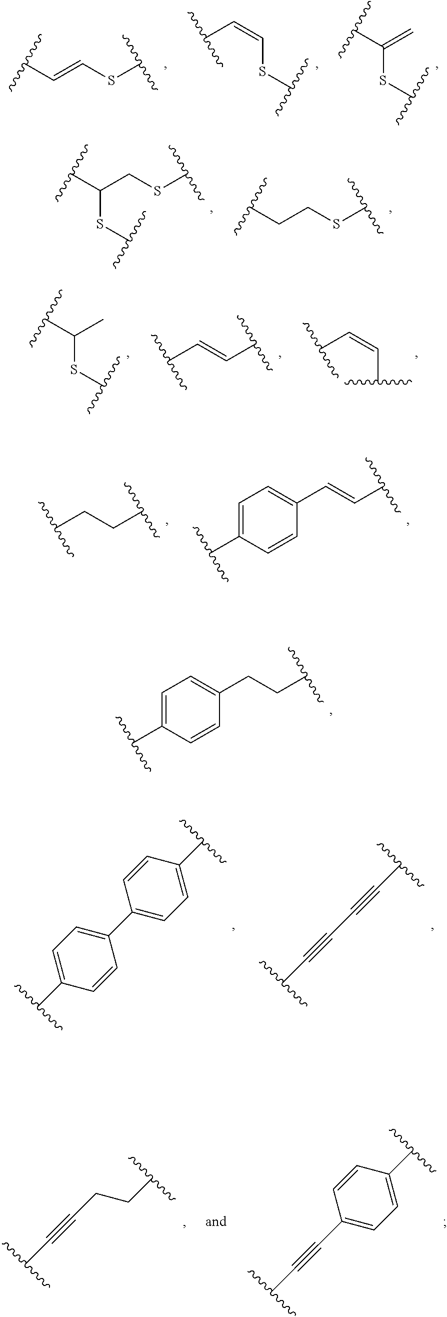

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In some embodiments, the solubility enhancing group SE for the above modular linker compounds (IIIb.1) to (IIIb.6) is selected from

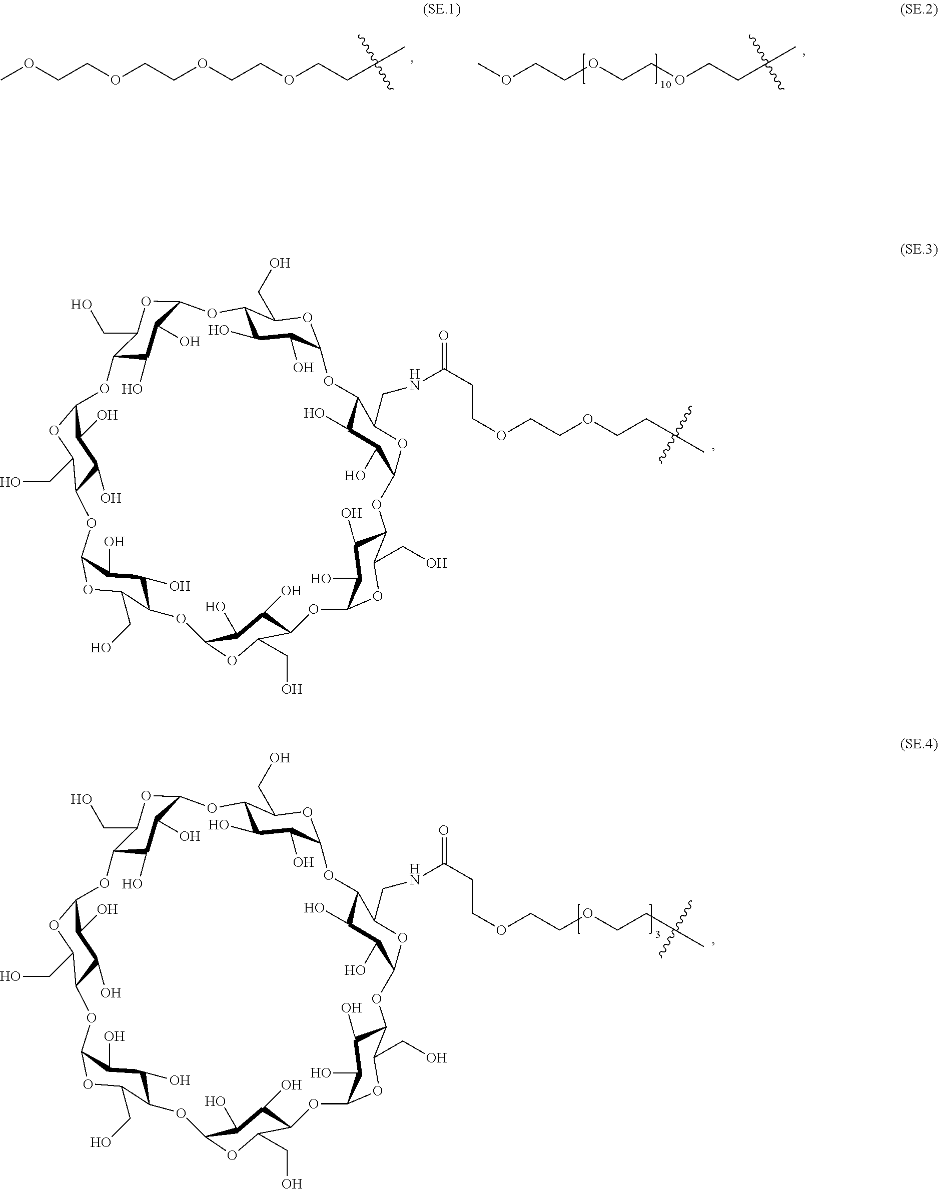

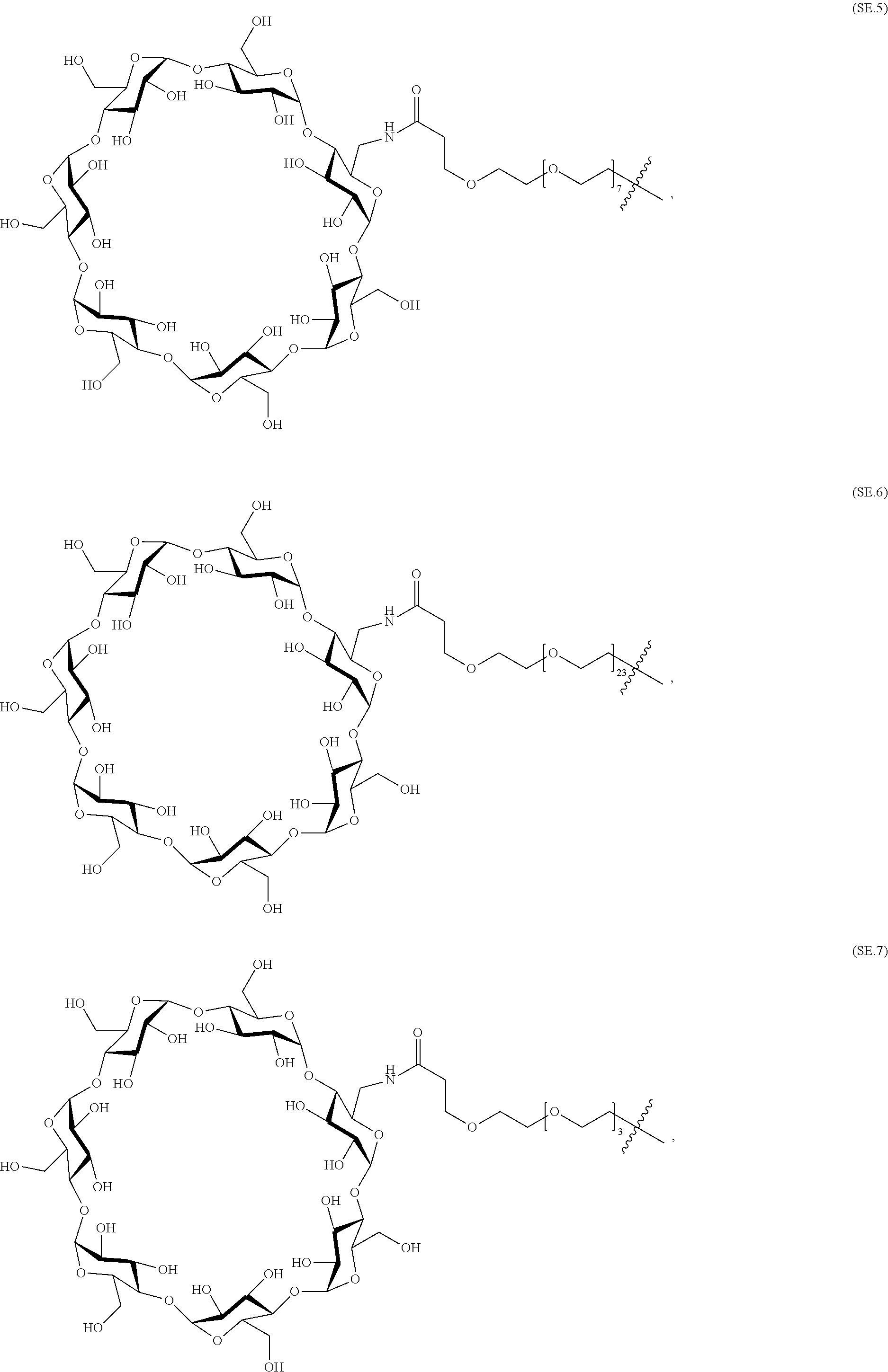

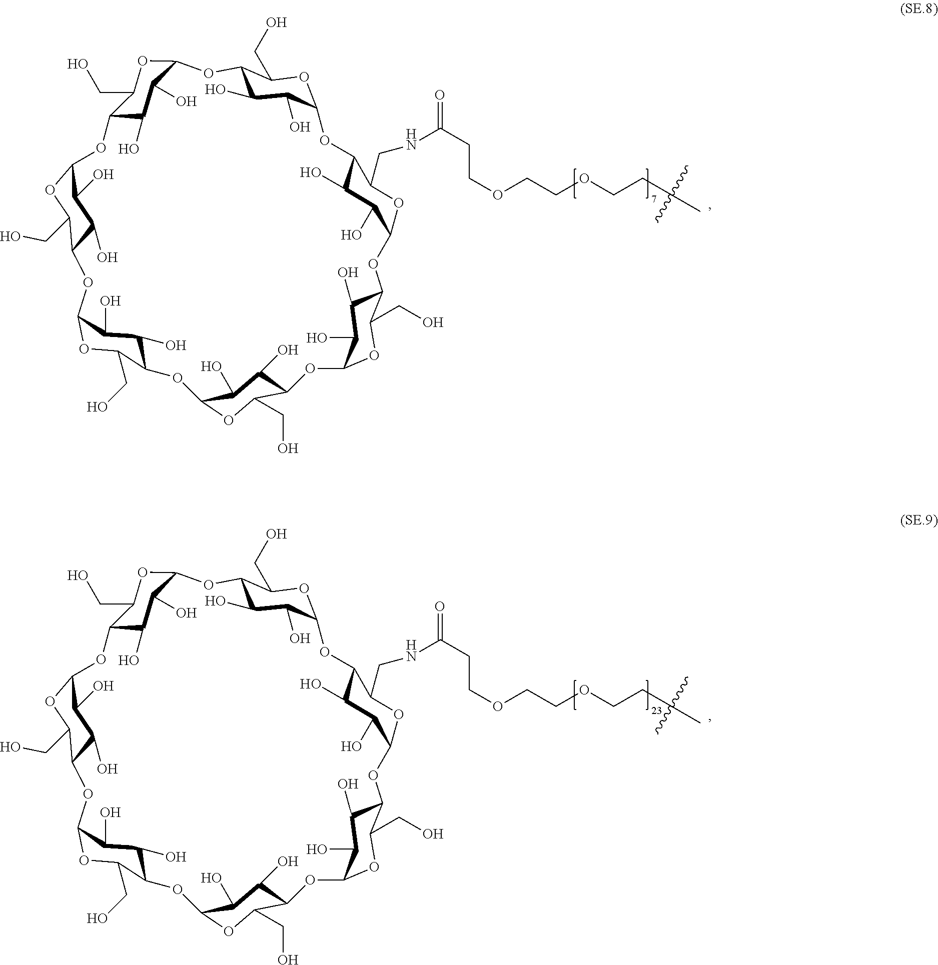

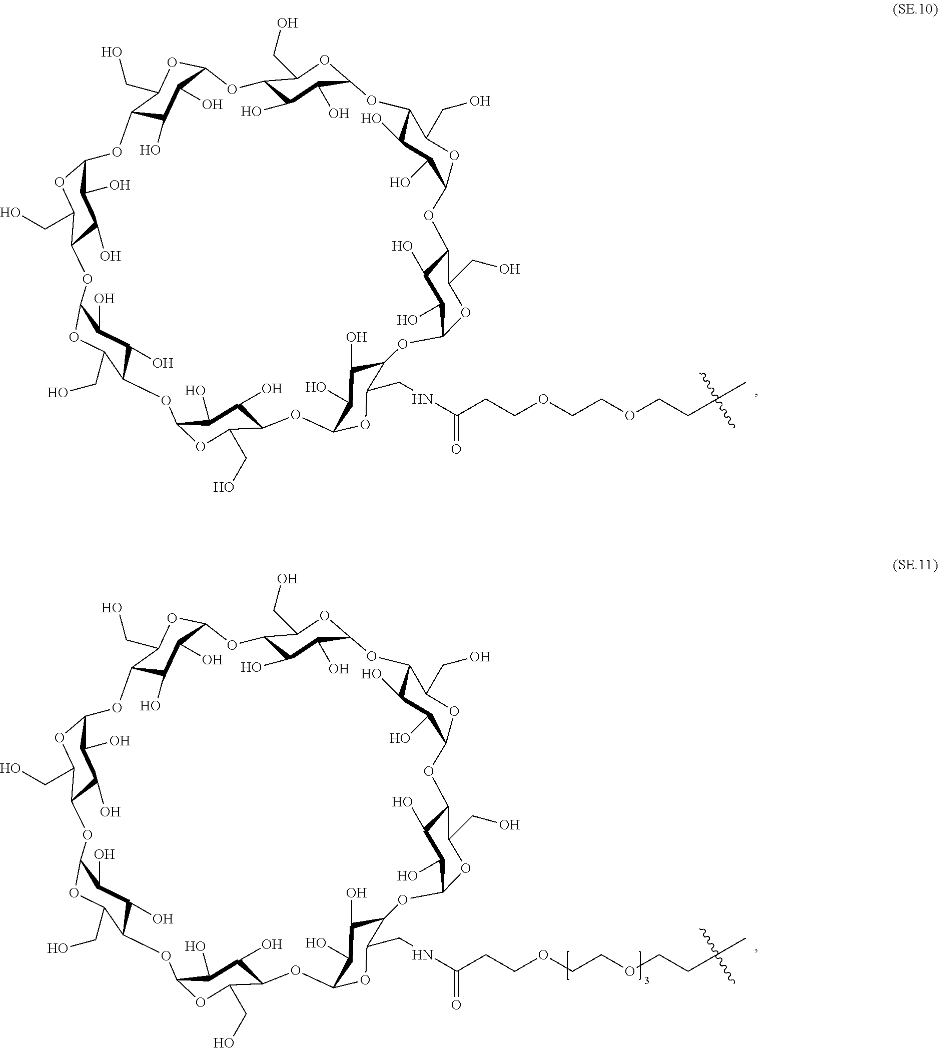

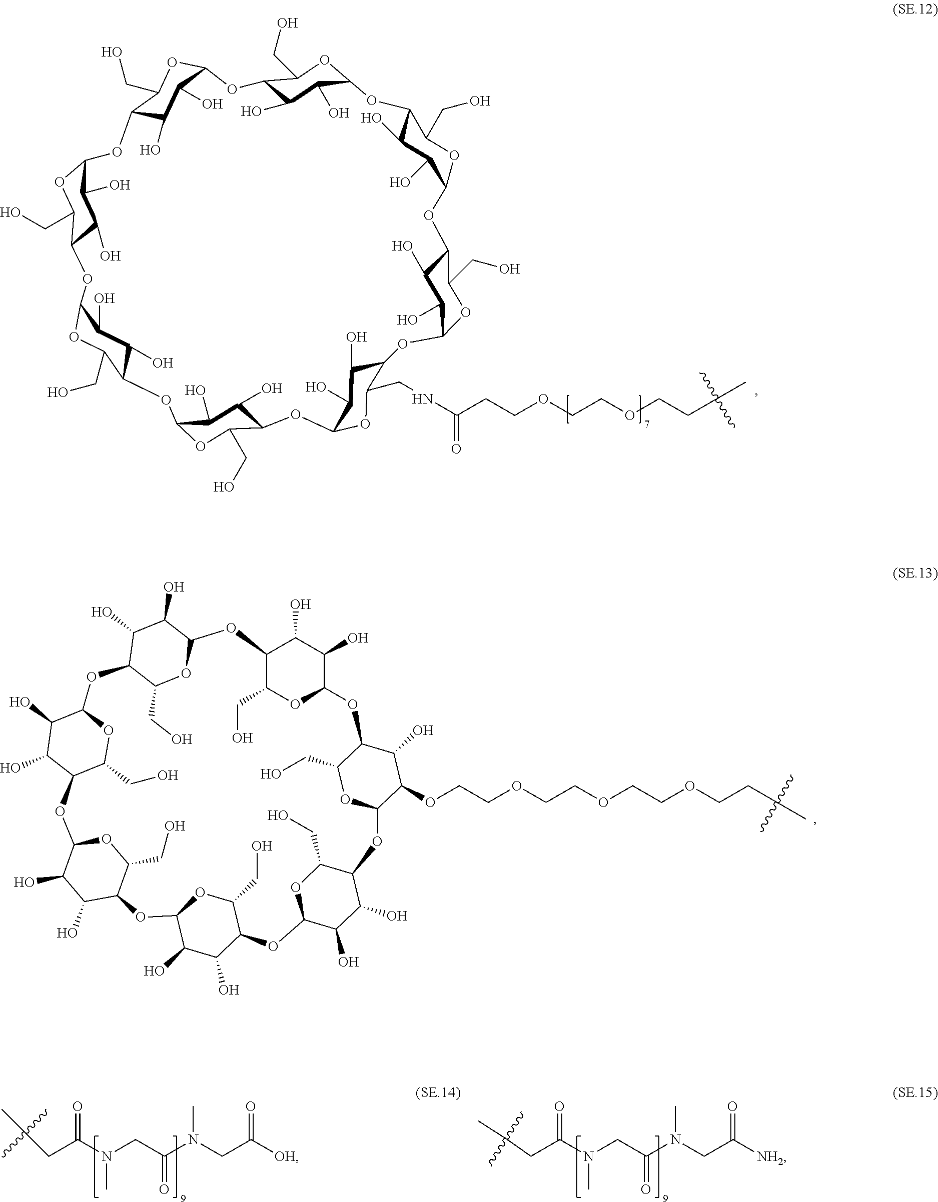

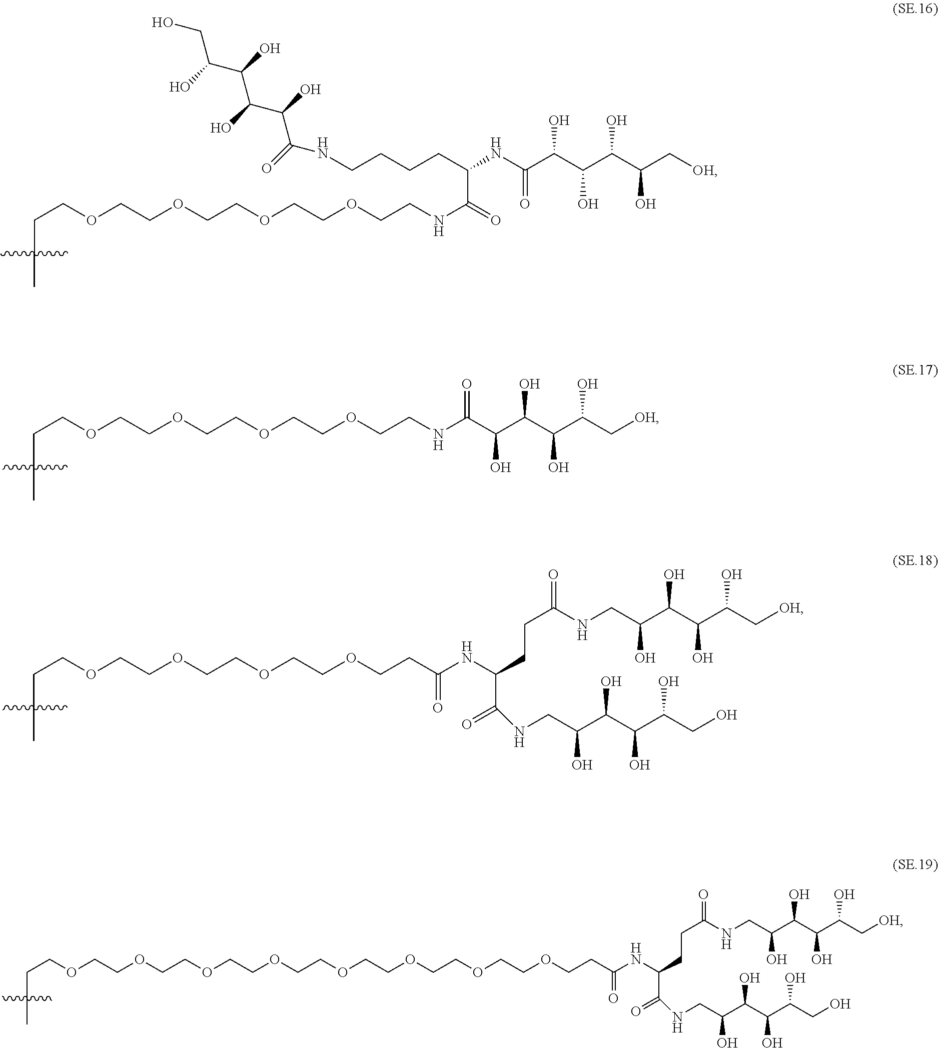 [SMILES: *CCOCCOCCOCCOCCNC(=O)[C@H](CCCCNC(=O)[C@H](O)[C@@H](O)[C@H](O)[C@H](O)CO)NC(=O)[C@H](O)[C@@H](O)[C@H](O)[C@H](O)CO ; *CCOCCOCCOCCOCCNC(=O)[C@H](O)[C@@H](O)[C@H](O)[C@H](O)CO ; *CCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO ; *CCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO]

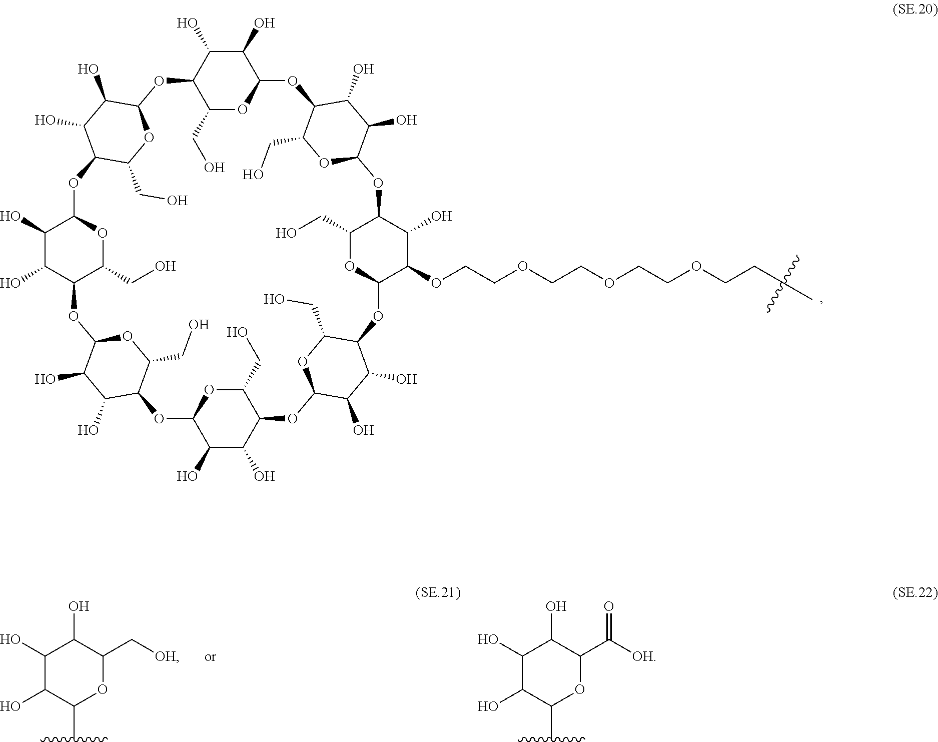 [SMILES: *CCOCCOCCOCCO[C@H]1[C@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@@H]([C@@H]1O)[C@@H](CO)O2)O[C@@H]9CO)O[C@@H]8CO)O[C@@H]7CO)O[C@@H]6CO)O[C@@H]5CO)O[C@@H]4CO)O[C@@H]3CO ; *C1OC(CO)C(O)C(O)C1O ; *C1OC(C(=O)O)C(O)C(O)C1O]

In some embodiments the modular linker compounds of the present disclosure are:

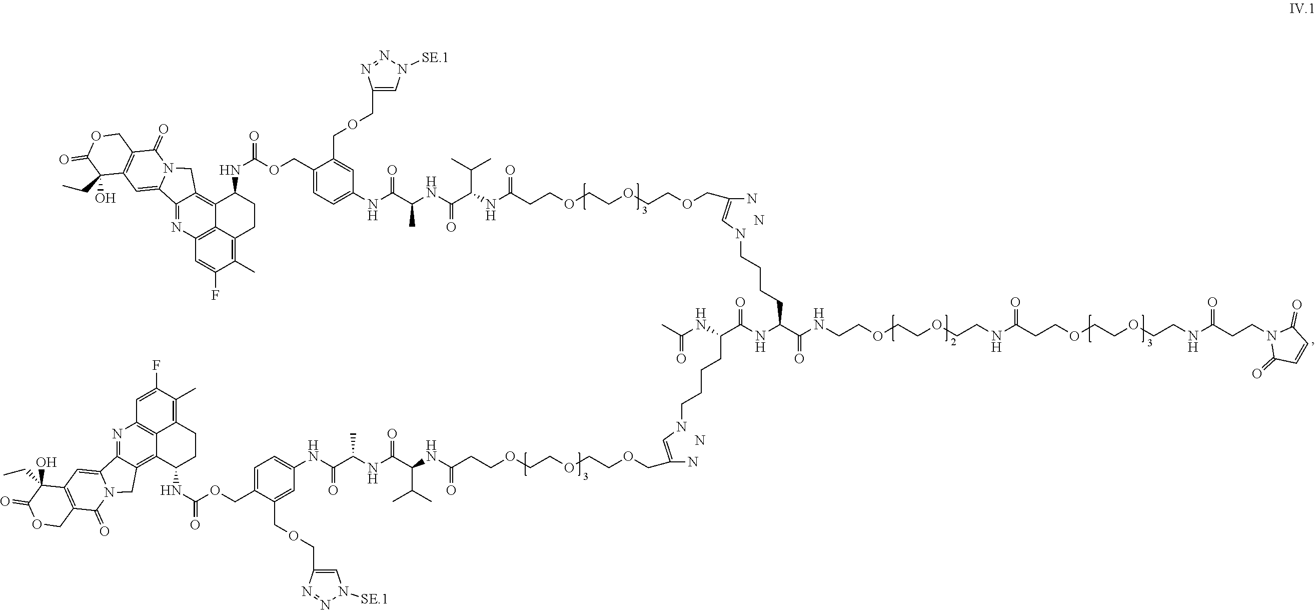

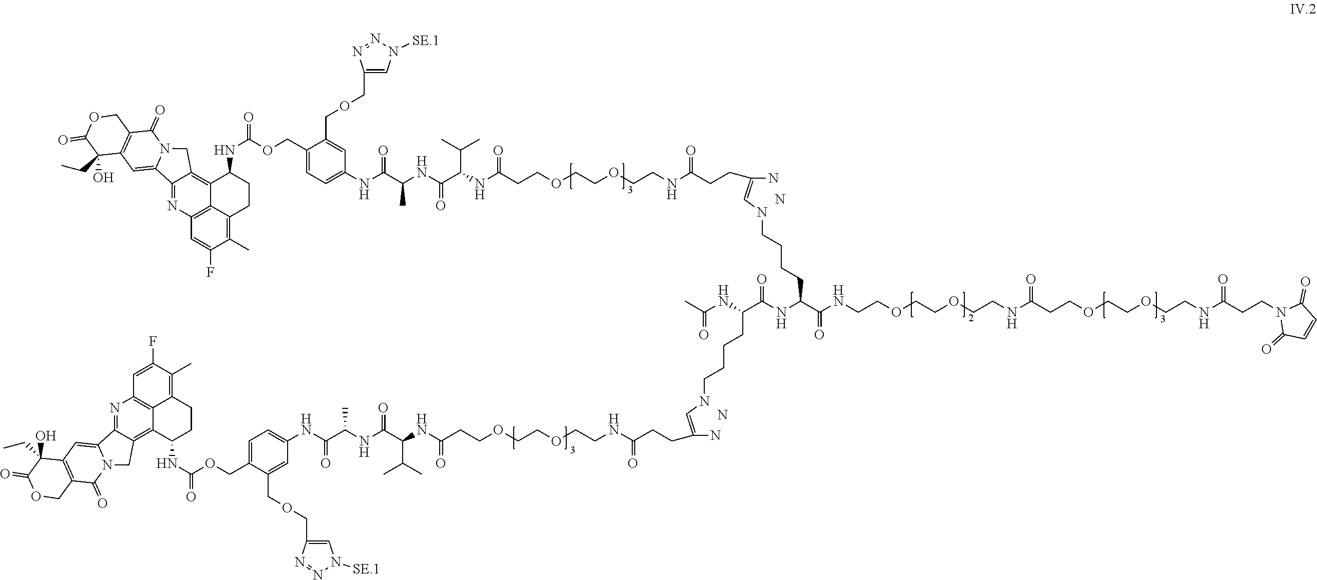

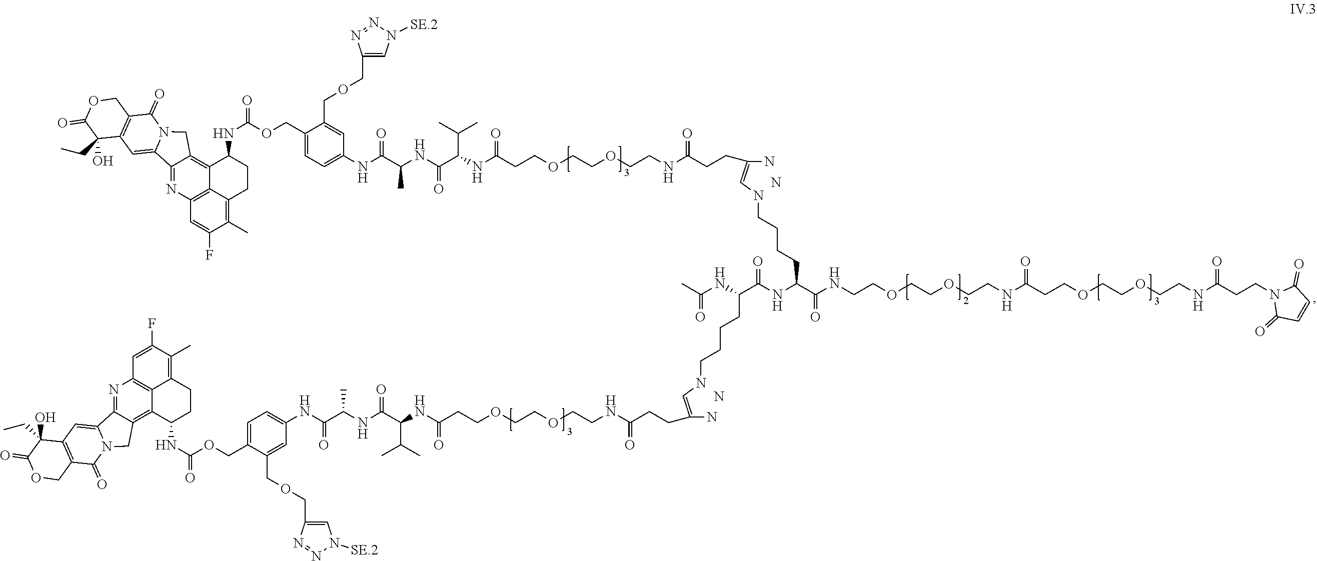

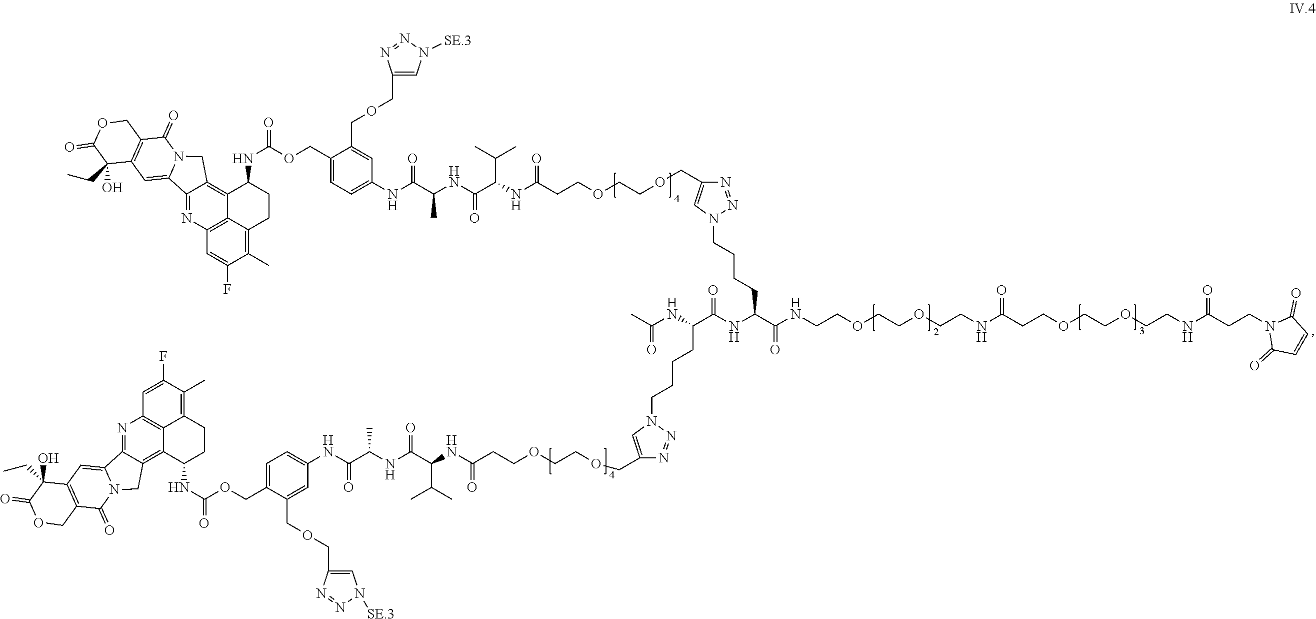

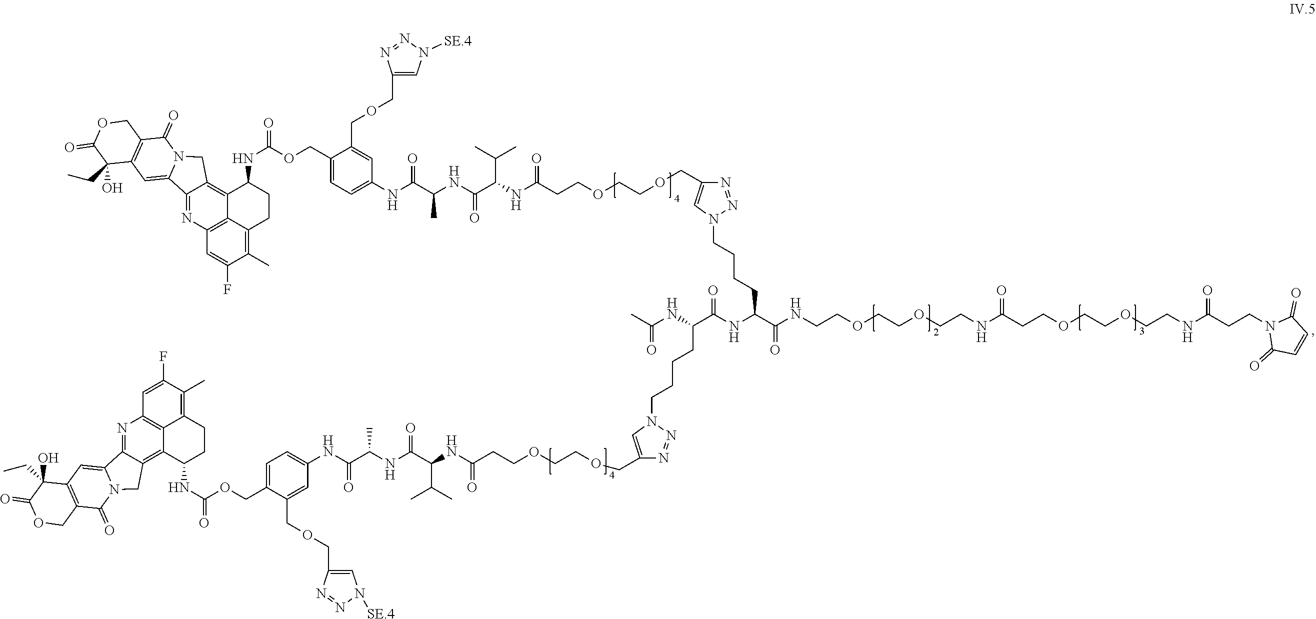

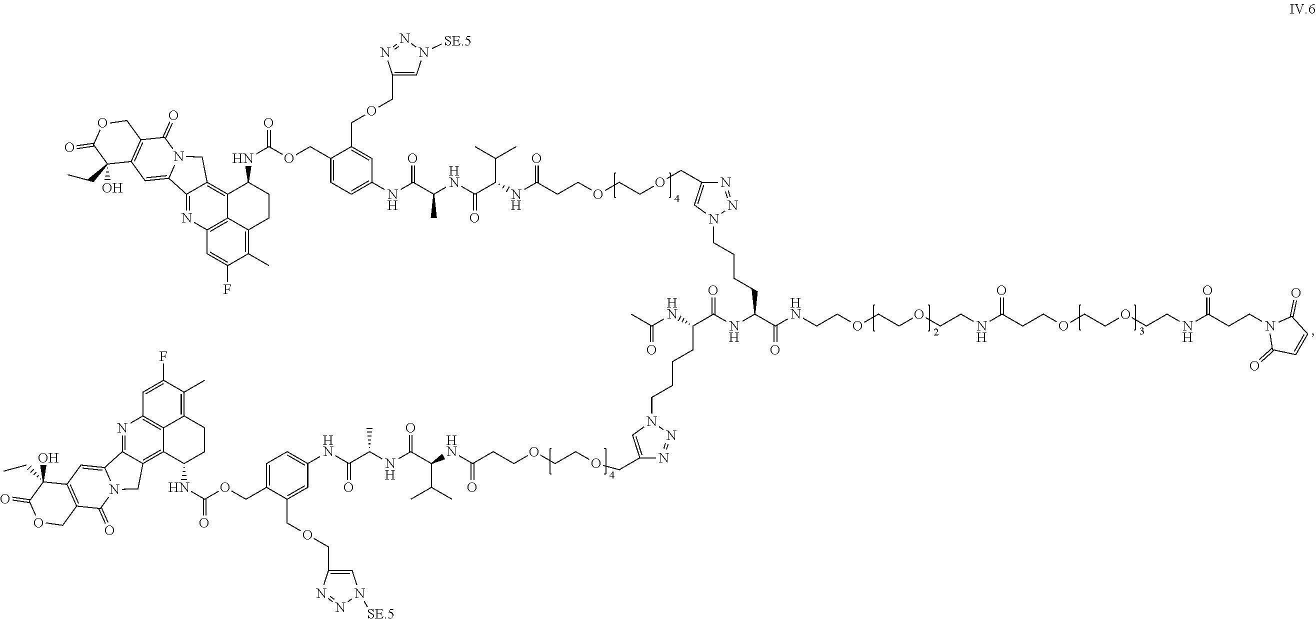

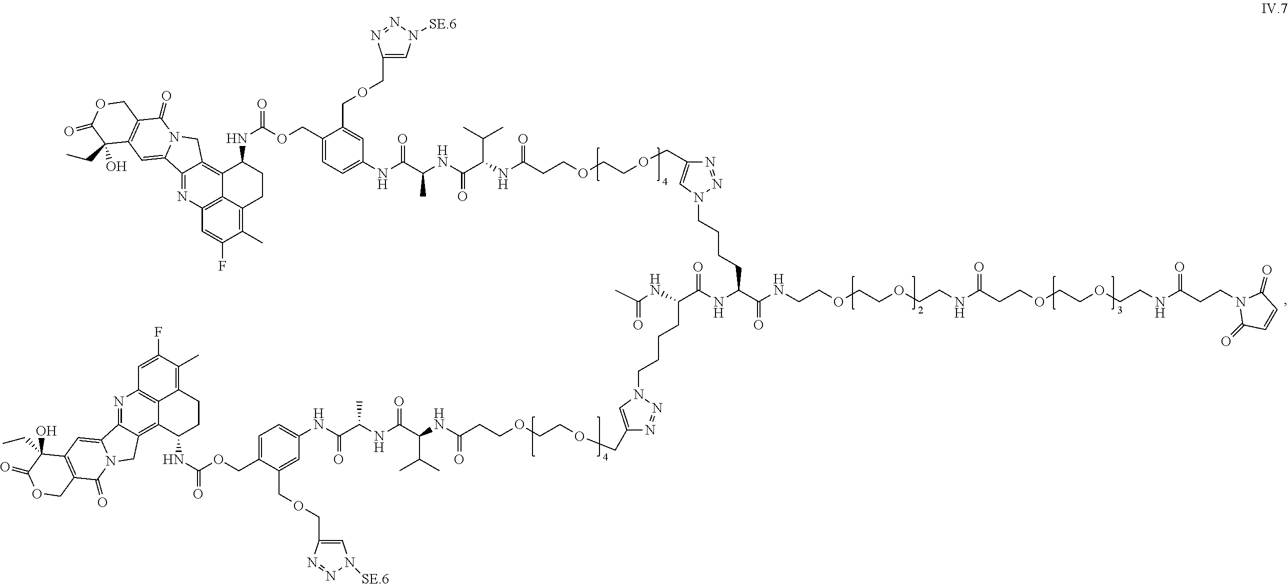

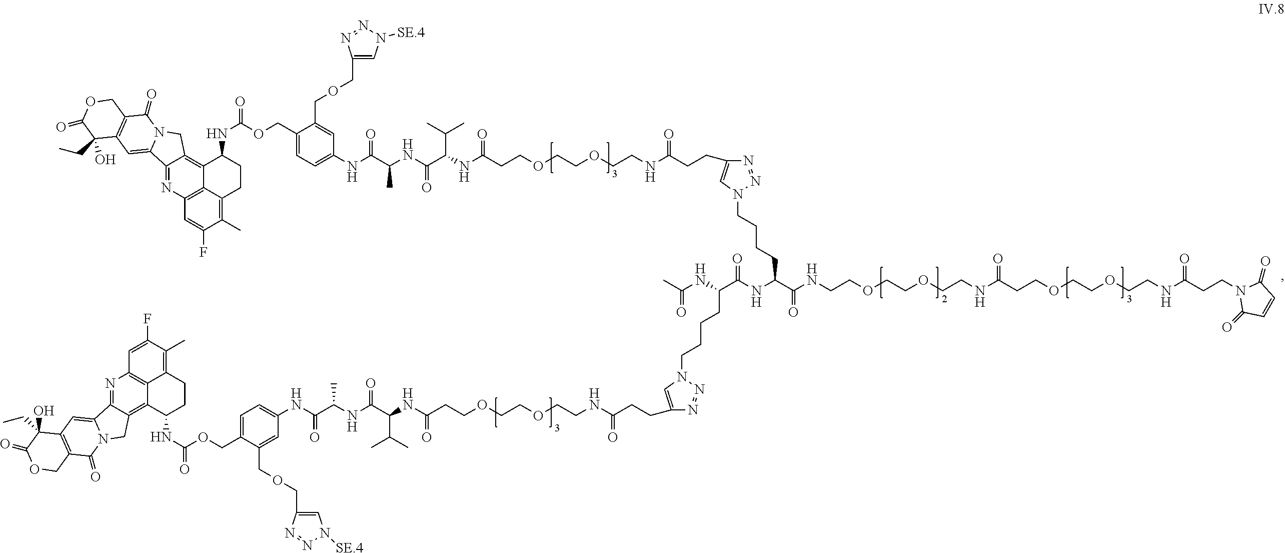

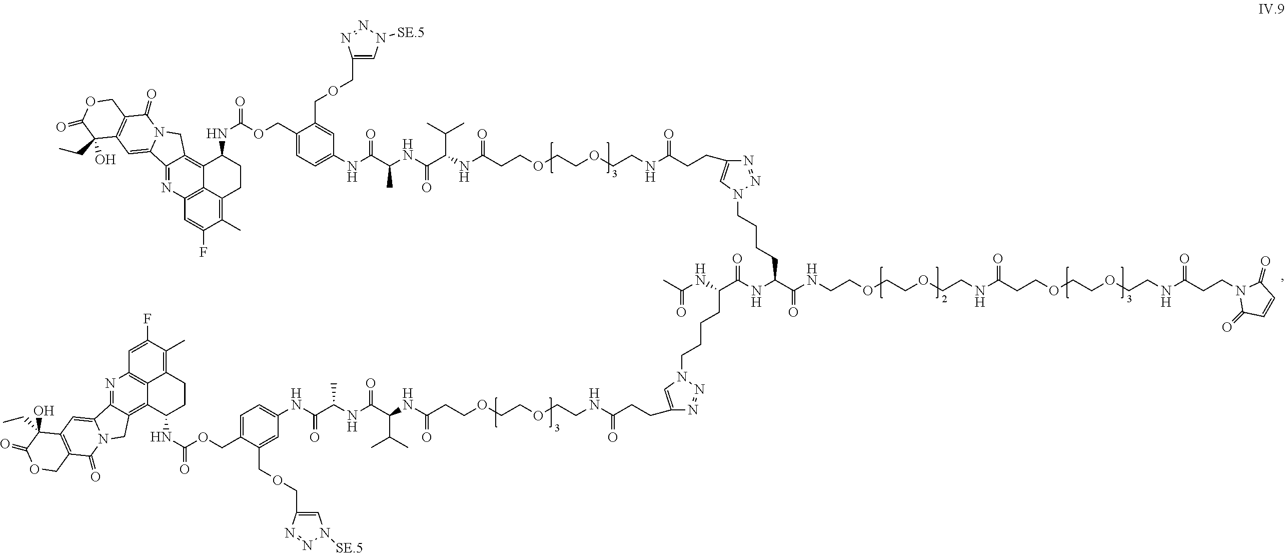

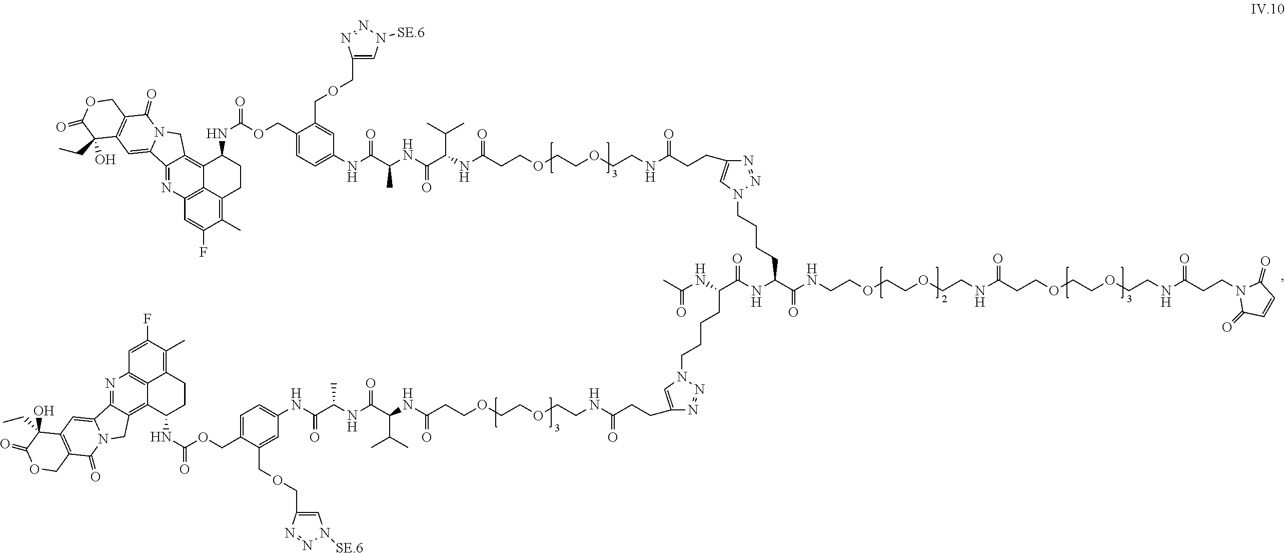

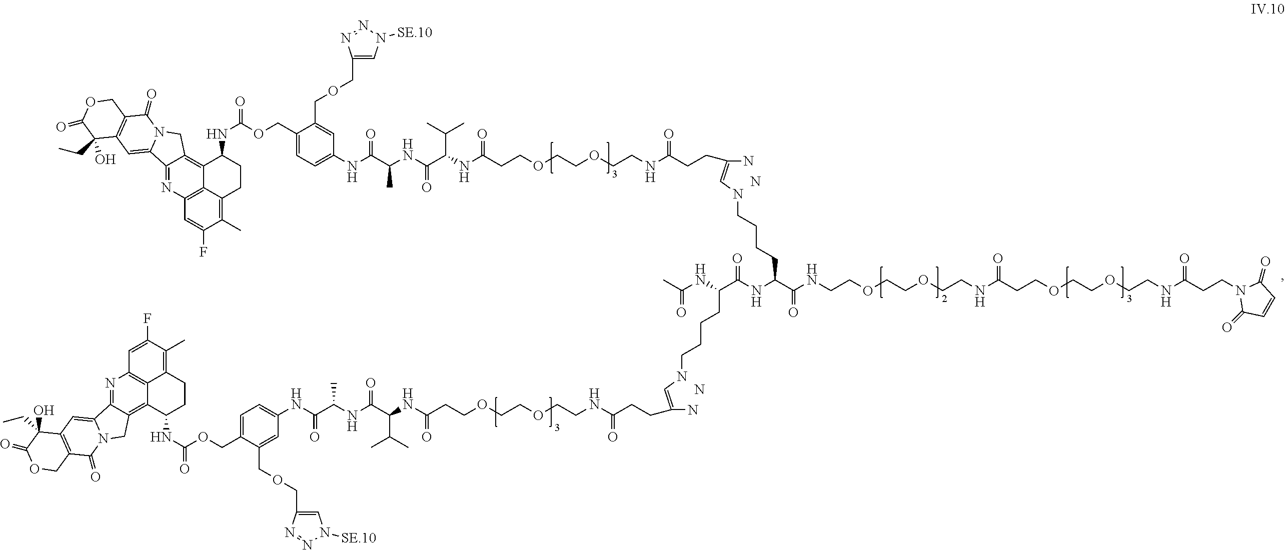

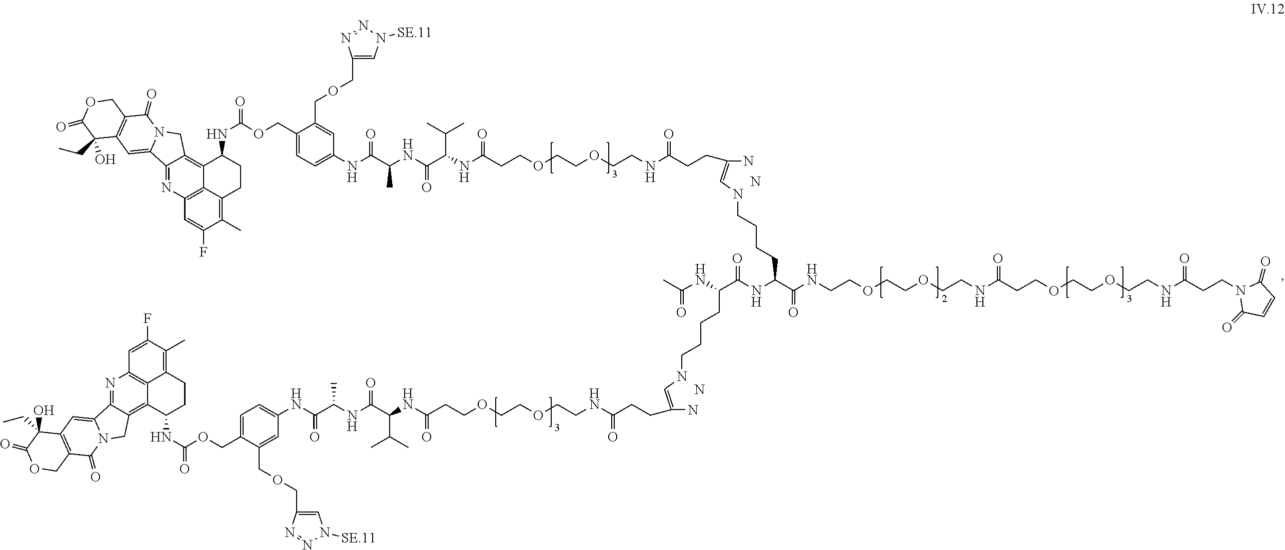

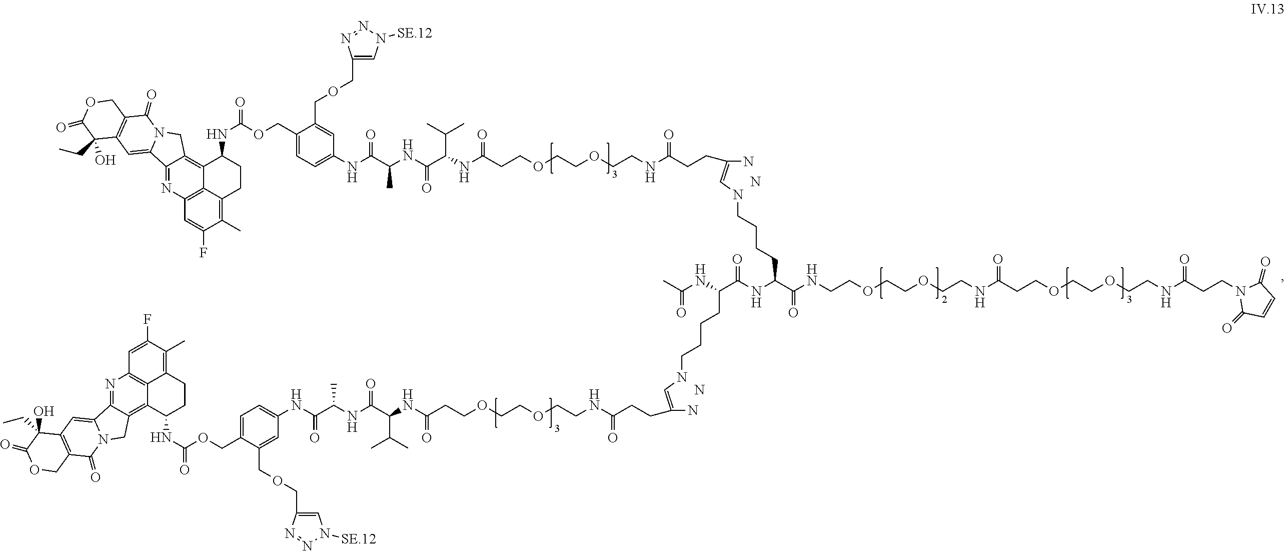

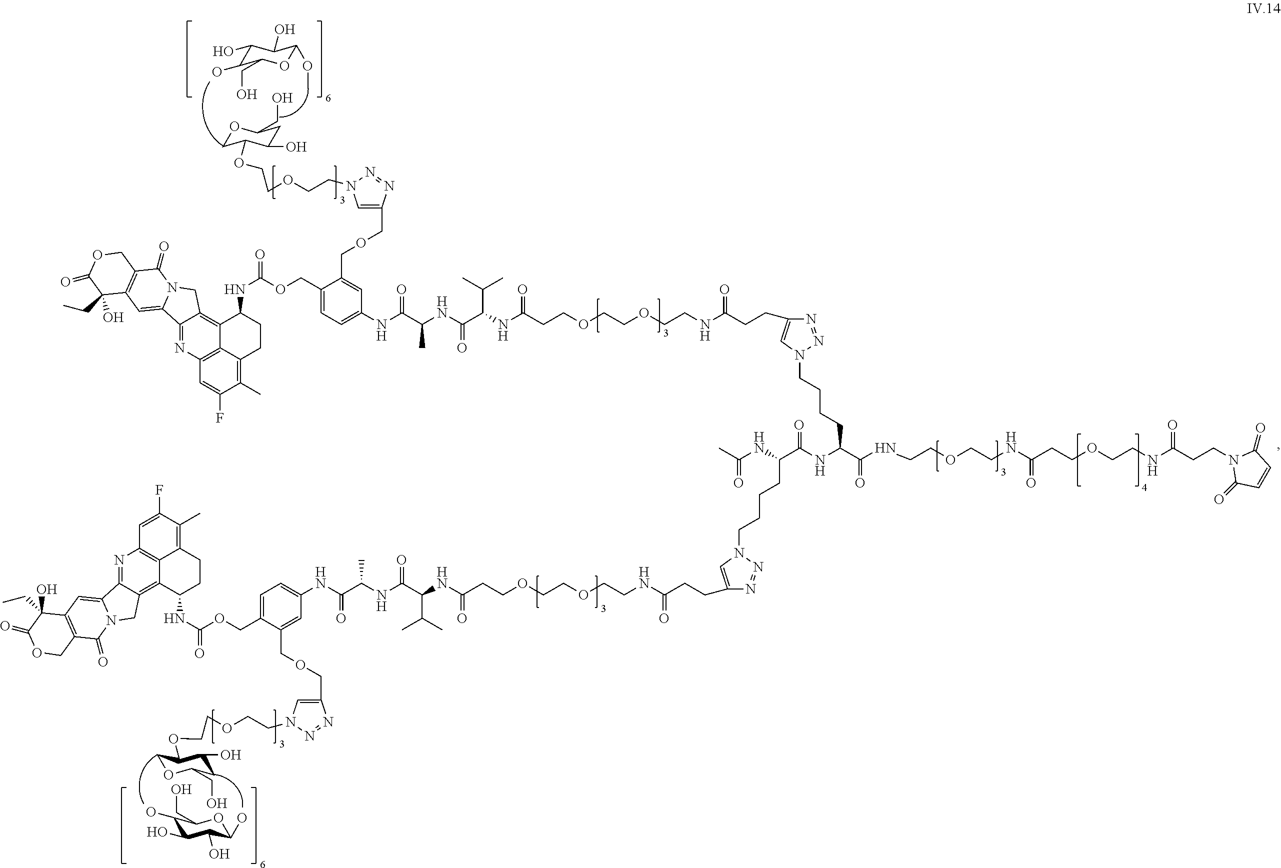

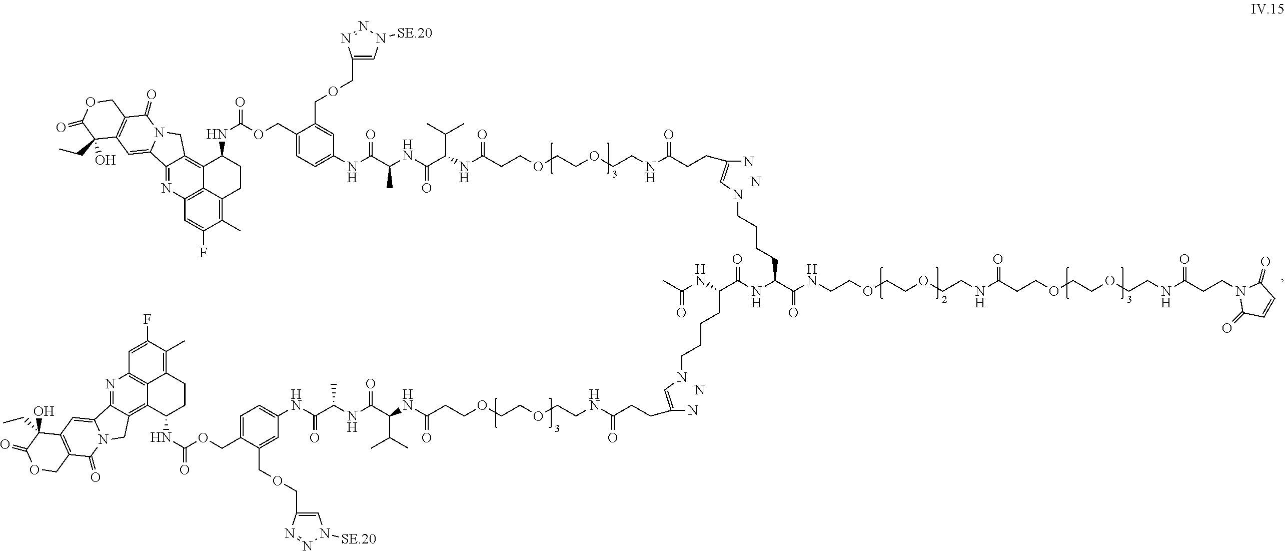

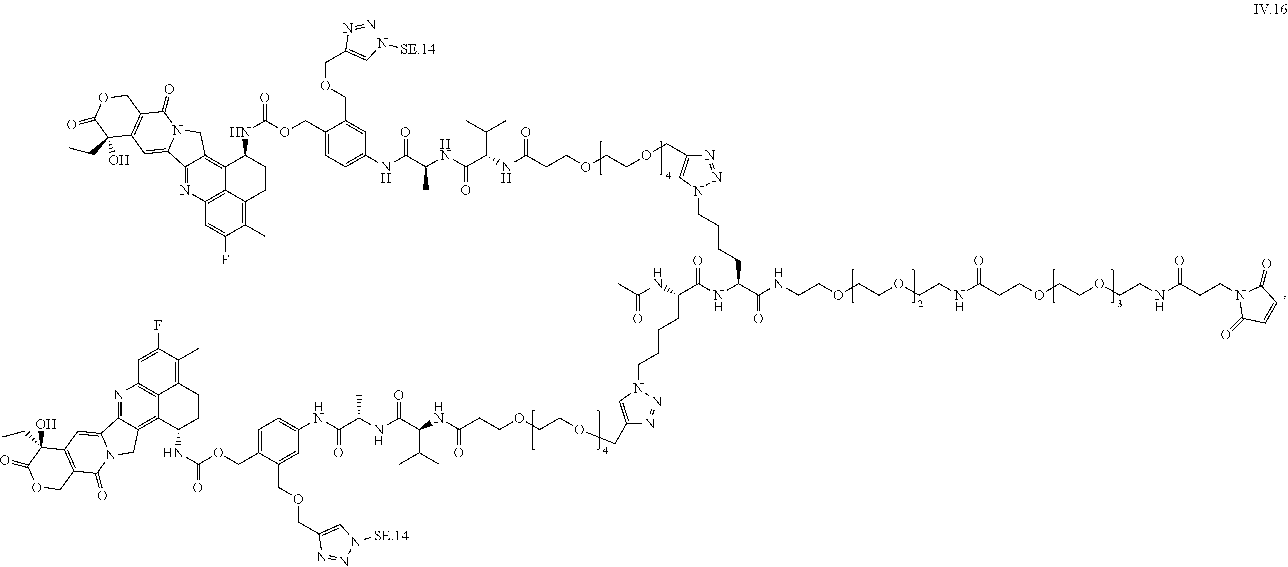

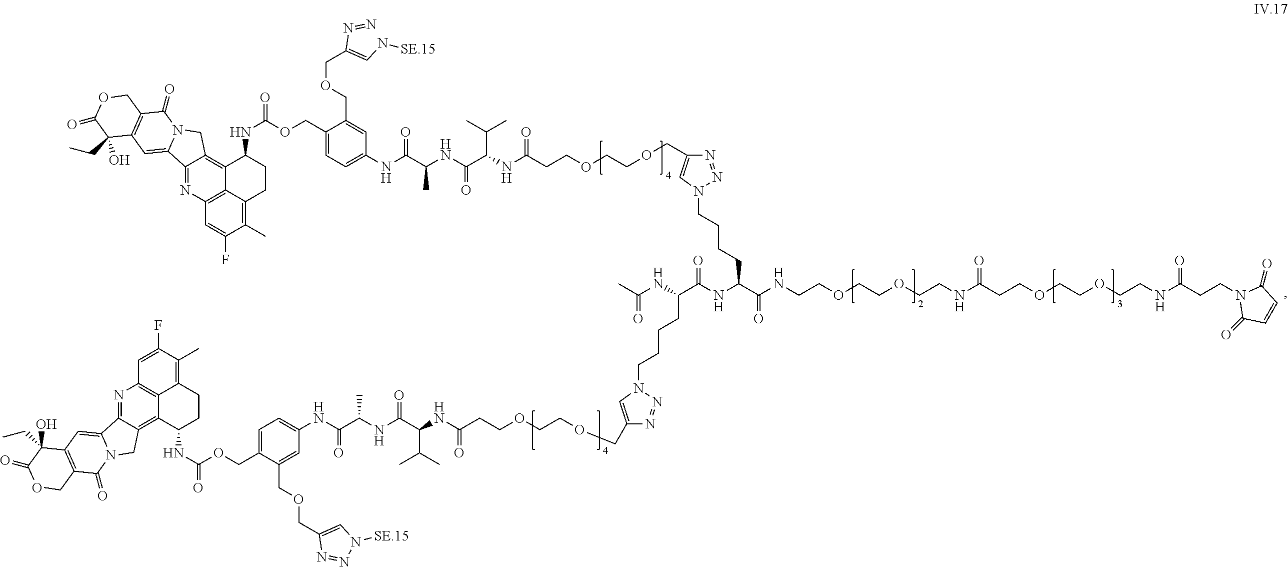

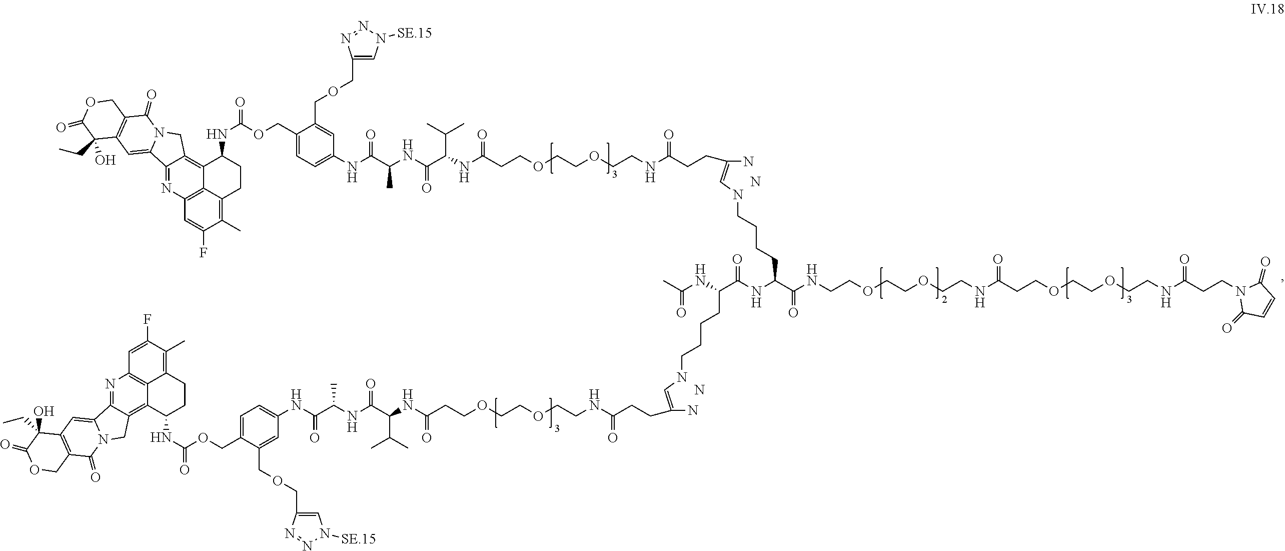

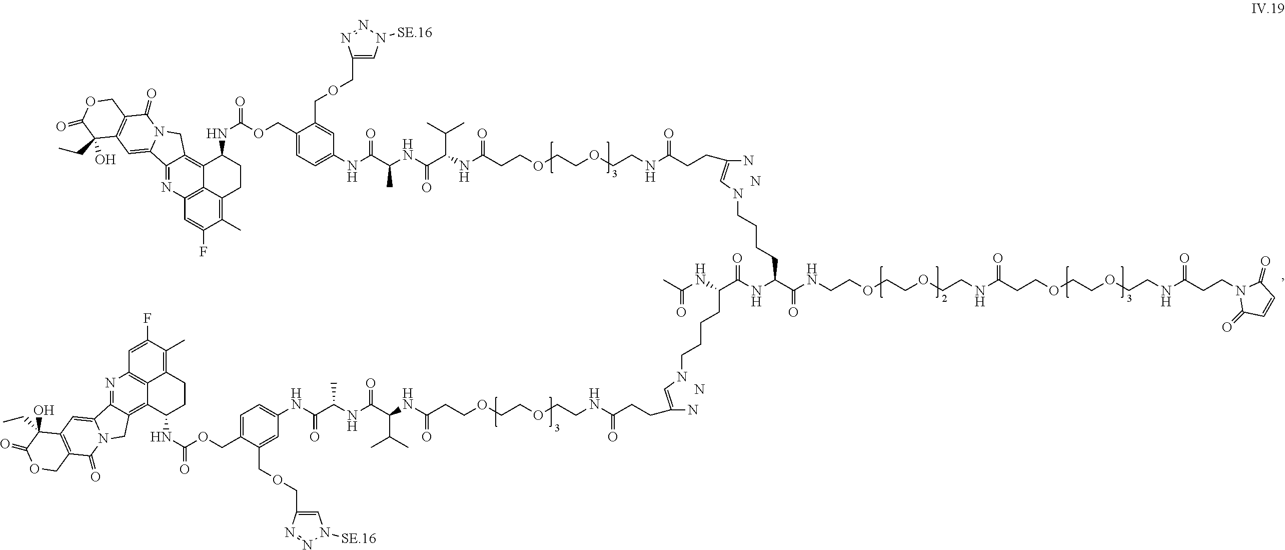

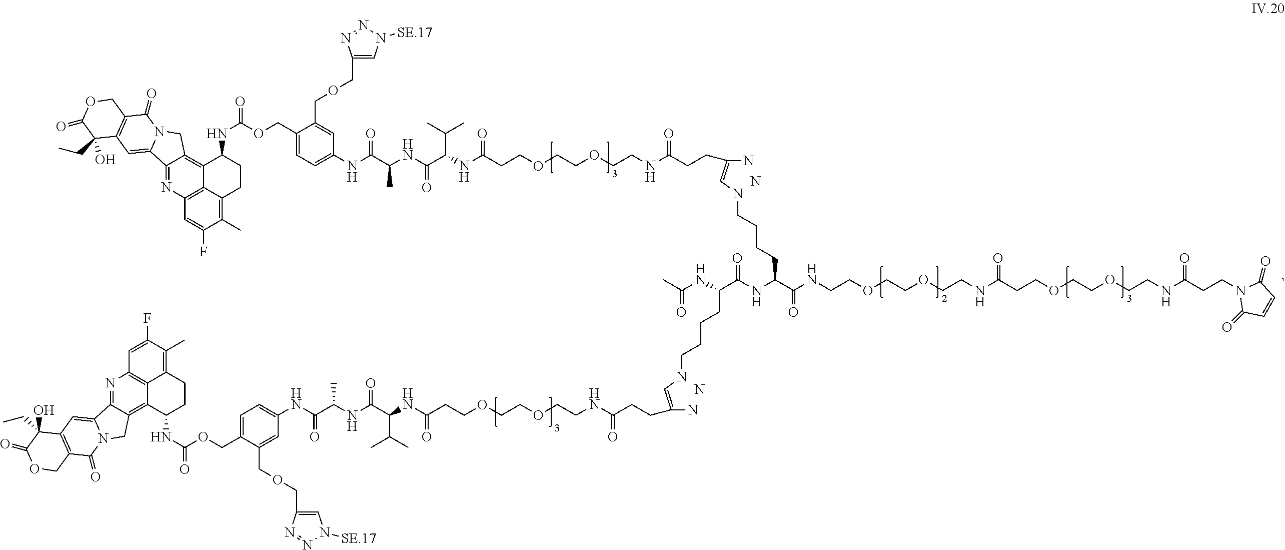

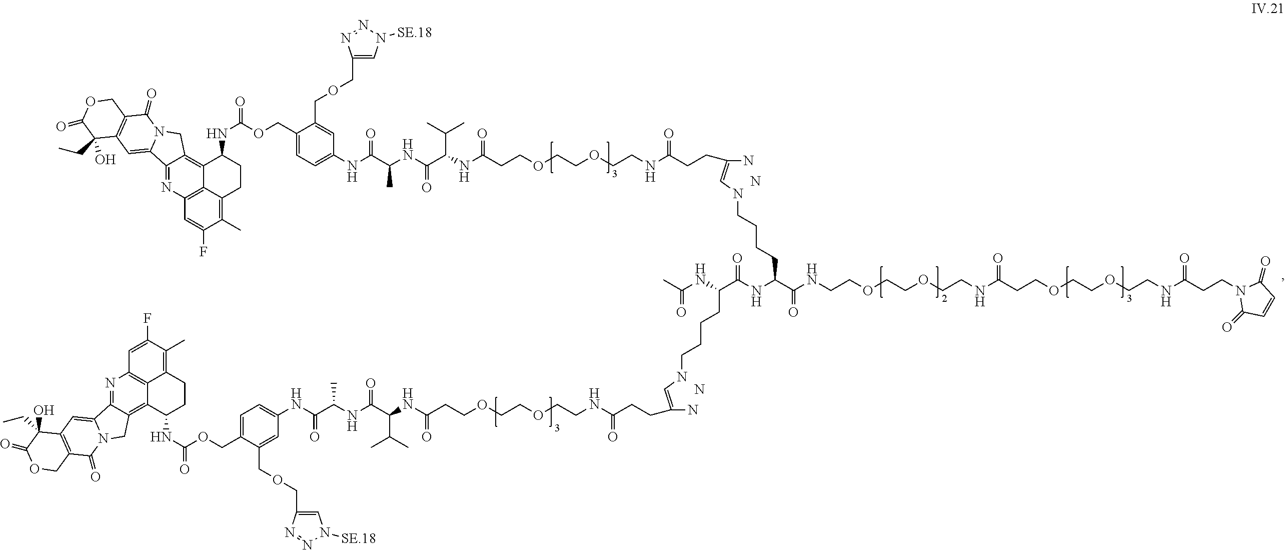

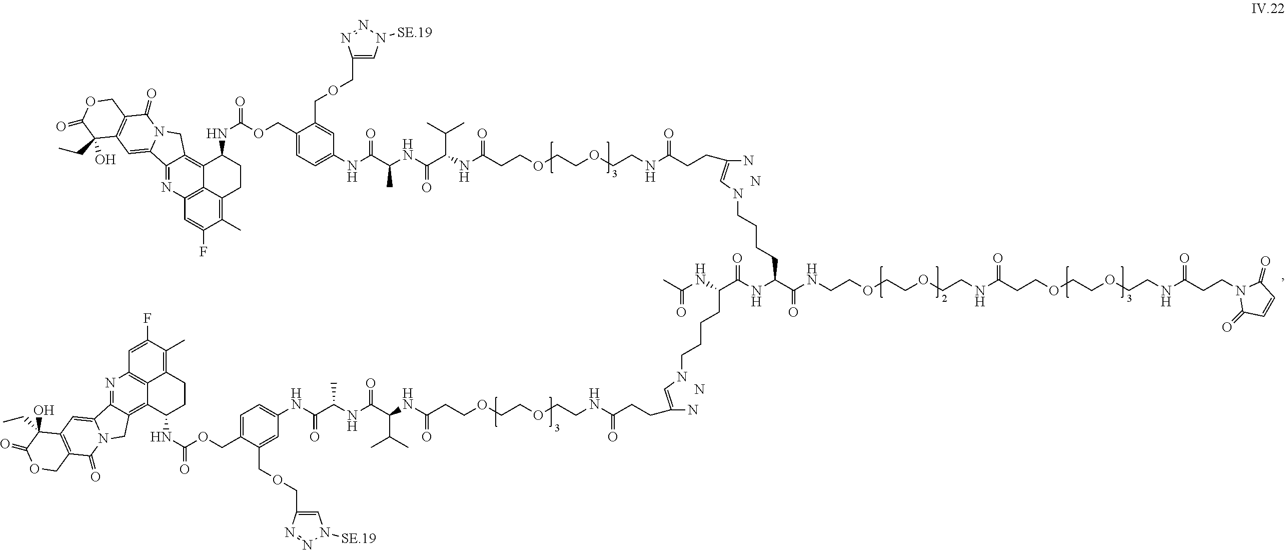

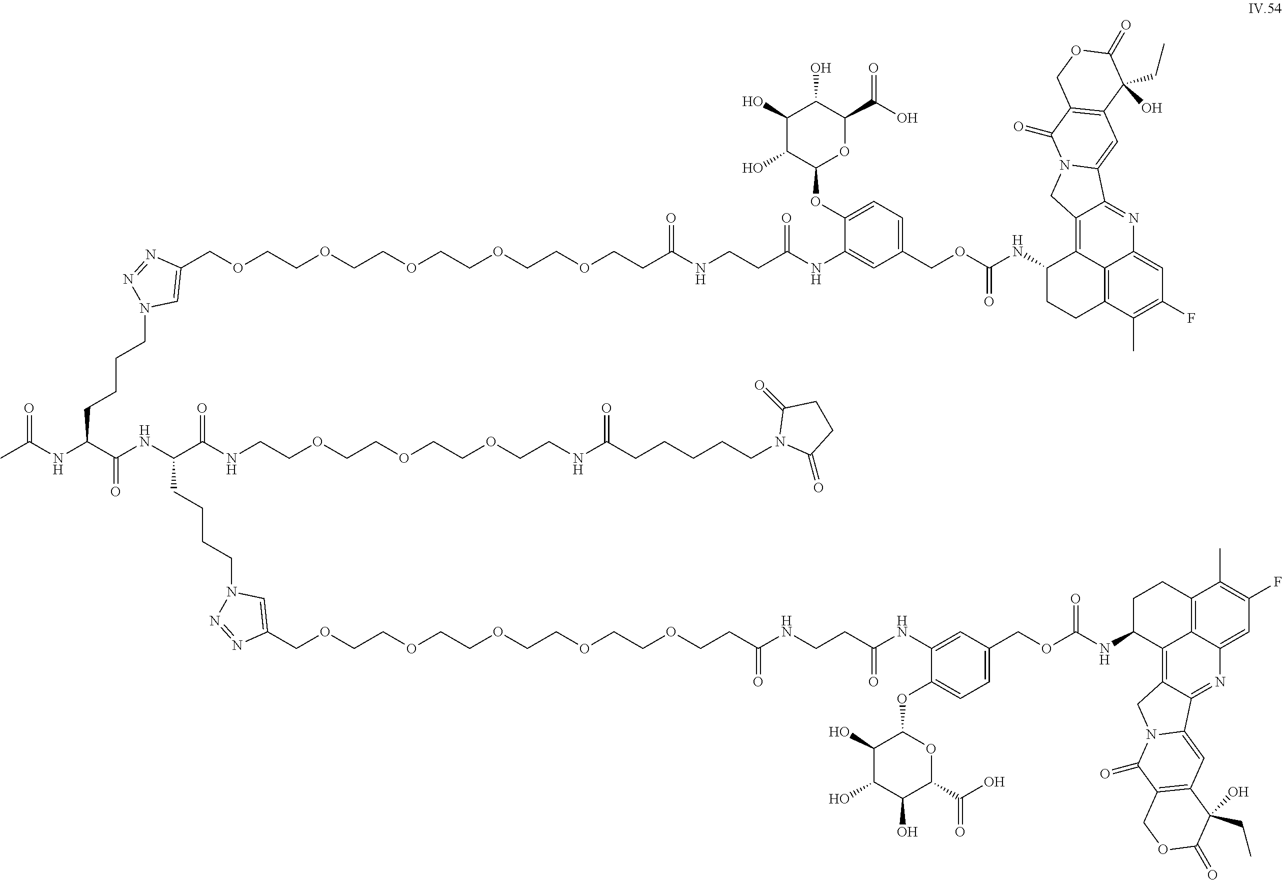 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CCNC(=O)CCOCCOCCOCCOCCOCc2cn(CCCC[C@H](NC(C)=O)C(=O)N[C@@H](CCCCn4cc(COCCOCCOCCOCCOCCC(=O)NCCC(=O)Nc5cc(COC(=O)N[C@H]6CCc7c(C)c(F)cc8nc9c(c6c78)Cn6c-9cc7c(c6=O)COC(=O)[C@]7(O)CC)ccc5O[C@@H]5O[C@H](C(=O)O)[C@@H](O)[C@H](O)[C@H]5O)nn4)C(=O)NCCOCCOCCOCCNC(=O)CCCCCN4C(=O)CCC4=O)nn2)c1)CC3]

The inventors have further realized that the modular linker compound of the first aspect can be synthesized in a particularly advantageous high-yield method utilizing building blocks, which reliably can be assembled into the modular linker compounds of the present disclosure via robust, predictable and orthogonal CLICK-chemistry reactions.

Accordingly, in a second aspect, the present disclosure relates to a method for synthesizing a modular linker compound of Formula (I) according to the first aspect, the method comprising:
(i) Contacting a first compound comprising:
   an immolative moiety,
   a cleavable peptide sequence W,
   an amine protecting group Pg¹, which is covalently linked to W,
   a pharmaceutically active substance D and
   a first complementary group Cg¹
with a second compound comprising
   a solubility enhancing group SE and
   a second complementary group Cg²
to form an intermediate of Formula II:

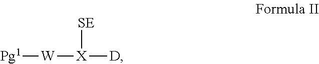

wherein SE is connected to X via R² and wherein R² is formed by the reaction of Cg¹ and Cg²;
(ii) Removing the amine protecting group Pg¹ from Formula II to expose a primary amine;
(iii) Contacting said primary amine with a third compound comprising
   a spacer Z³,
   an amine reactive group, and
   a third complementary group Cg³
to form the intermediate of Formula III:

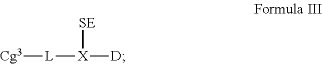

wherein L is

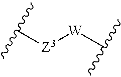

(iv) Contacting the intermediate of Formula III with a branched fourth compound comprising
   a fourth complementary group Cg⁴ and
   a fifth complementary group Cg⁵,
wherein Cg³ reacts with Cg⁴ to form Z²;
(v) Contacting the product of (iv) with a fifth compound comprising
   a reactive moiety A for conjugation to an target-binding moiety and
   a sixth complementary group Cg⁶
wherein reaction of Cg⁵ and Cg⁶ forms Z¹, to form the modular linker compound of Formula I:

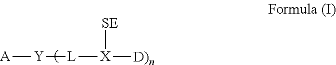

In embodiments of the method for synthesizing a modular linker compound of Formula (I) of the second aspect (including in the subsequently described embodiments of this method of the second aspect),
Cg¹ can be an alkyne group and Cg² can be an azide group, or
Cg¹ can be a primary or secondary amine group and Cg² can be a carboxylic acid, carboxylic ester optionally N-hydroxysuccinimide ester, tetrafluorophenyl ester, pentafluorophenyl ester, or N-hydroxyphtalimide ester, carboxylic anhydride, acyl halide or amide group.

Further, in embodiments of the method for synthesizing a modular linker compound of Formula (I) of the second aspect (including in the subsequently described embodiments of this method of the second aspect),
Cg³ can be an alkyne group and Cg⁴ can be an azide group;
Cg³ can be an azide group and Cg⁴ can be an alkyne group;
Cg³ can be an primary or secondary amine group and Cg⁴ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group; or
Cg³ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group and Cg⁴ can be a primary or secondary amine group.

In further embodiments of the method for synthesizing a modular linker compound of Formula (I) of the second aspect (including in the subsequently described embodiments of this method of the second aspect),
Cg⁵ can be a primary or secondary amine group and Cg⁶ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group; or
Cg⁵ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group and Cg⁶ can be a primary or secondary amine group.

In some embodiments of the method for synthesizing a modular linker compound of Formula (I) of the second aspect (including in the subsequently described embodiments of this method of the second aspect), the amine protecting group Pg¹ can be a carbobenzyloxy (Cbz), tert-butyloxycarbonyl (Boc), 9-fluorenylmethyloxycarbonyl (Fmoc), acetyl (Ac), benzoyl (Bz), benzyl (Bn), p-bethoxybenzyl (PMB), p-toluenesulfonyl (Ts) or a carbamate group.

As will be described in more detail in the Examples below, in some embodiments, the well-known copper-catalyzed azide-alkyne cycloaddition click chemistry reaction (CuAAC reaction) is employed highly efficiently connect various building blocks of the modular linker compound of Formula (I) during the steps of the method for synthesizing the modular linker compound of the second aspect.

In this method for synthesizing a modular linker compound of Formula (I),
X is a self-immolative spacer

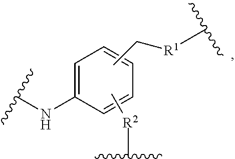

wherein:
R¹ covalently links X to D in para, meta or ortho to the amino group of X and is a bond or

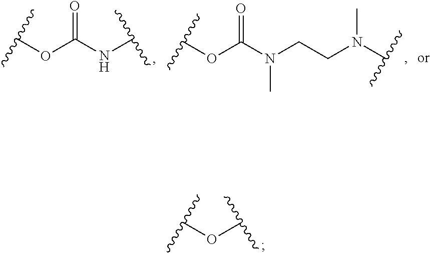

and
R² connects X to SE and is

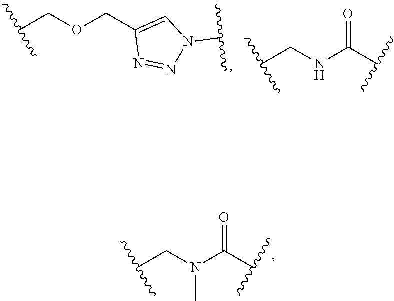

### or —O—;

In some embodiments, the cleavable or the non-cleavable peptide sequence W is selected from the group consisting of:
Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gln, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gln, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and
wherein Z³ is a bond or a spacer comprising a C₁₋₂₀ alkyl chain or a 1 to 10 polyethylene glycol (PEG)₁₋₁₀ or a C₂-amide-PEG₁₋₁₀;
wherein L optionally is

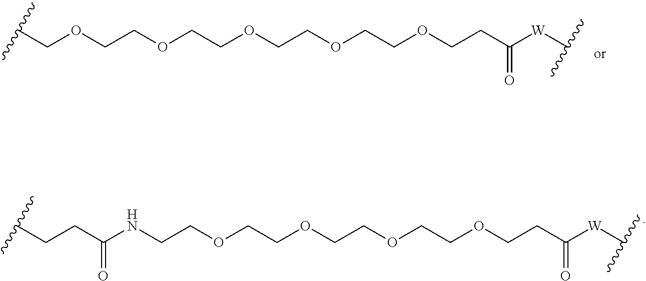

As described above, the branching unit Y of the modular linker compound of Formula (I)

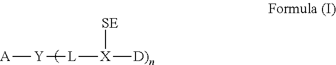

being synthesized in the method of the second aspect is:

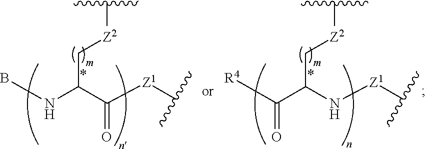

m is an integer from 1 to 6;
Z¹ connects Y to A and is

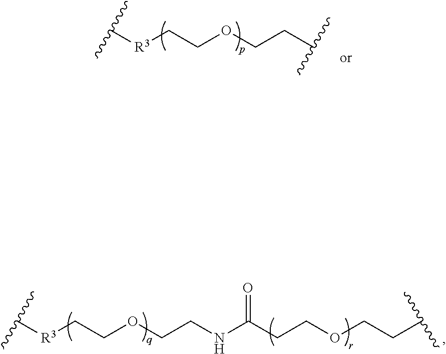

wherein
R³ is —O—, —NH—, —C(═O)—, —C(═O)N(CH₃)—, —C(═O) NH—, —NHC(═O)— or —N(CH₃) C(═O)—, and
p is an integer from 1 to 24, optionally from 4 to 24, optionally from 8 to 24, such as 8, 9, 10, 11, 12, 13, 14, 15, 16, 17, 18, 19, 20, 21, 22, 23 or 24;
q is an integer from 1 to 8, such as 1, 2, 3, 4, 5, 6, 7 or 8;
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
Z² connects Y to L and is a 1,2,3-triazole, an amide, or an urea derivative, selected from the group consisting of:

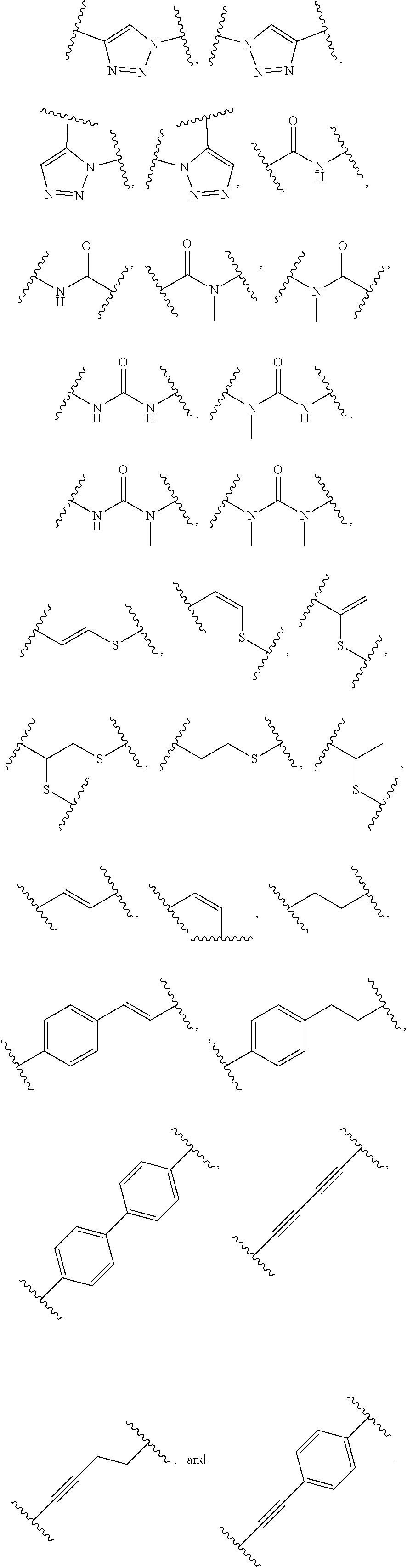

B is:
a cap, optionally an acyl group, and n′ is n; or

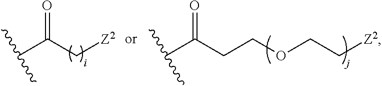

wherein
i is an integer from 1 to 20,
j is an integer from 1 to 10, and
n′ is n minus 1; or

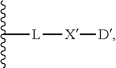

wherein
D′ is a pharmaceutically active substance different from D; and
X′ is self-immolative spacer

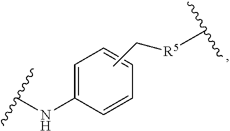

wherein:
R⁵ covalently links X′ to D′ in para or ortho to the amino group of X′ and is a bond,

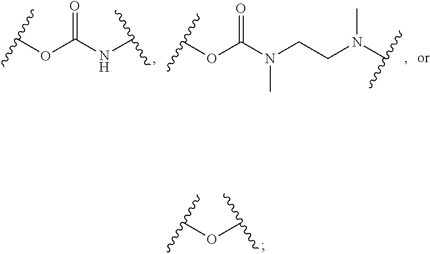

and n′ is n;
R⁴ is OH or NH₂.

If Z⁴ is selected from the group consisting of

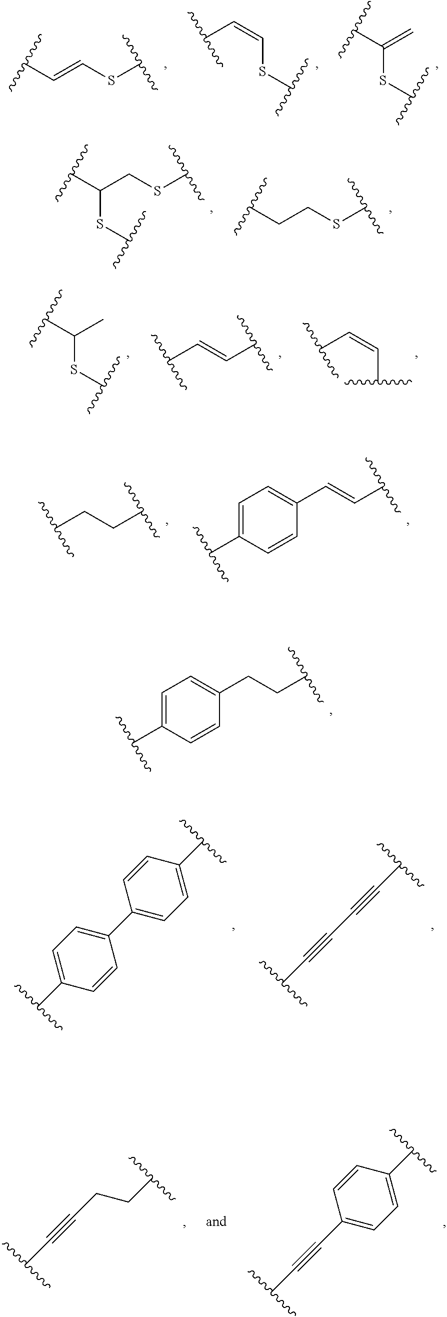

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In embodiments where B is

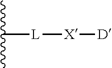

Z³ in L may further comprise a terminal carbonyl moiety, such as a dicarboxylic acid, for linkage of L to the amine group of Y.

Regardless of whether B is
a cap, optionally an acyl group, where n′ is n; or

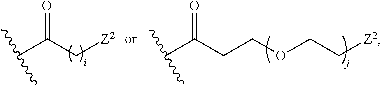

wherein
i is an integer from 1 to 20,
j is an integer from 1 to 10, and
n′ is n minus 1; or
a

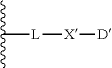

unit,
B constitutes the terminus of the final branching unit Y in multimeric core of the modular linker compound of Formula (I) synthesized in these embodiments of the second aspect.

As described above, the multimeric core in a modular linker compound of Formula (I) synthesized by the method of the second aspect can comprise n=2 to 5, optionally 2, 3, 4 or 5, branching units Y, wherein the branching unit proximal to A is connected to A via Z¹ and the final branching unit (i.e. the branching unit distal to A) comprises B. Each branching unit Y in this embodiment is indirectly linked to one molecule of a pharmaceutical drug substance D via spacer Z², spacer L and the immolative spacer X.

In alternative embodiments, branching unit Y of the modular linker compound of the Formula (I) being synthesized in this method of the second aspect is:

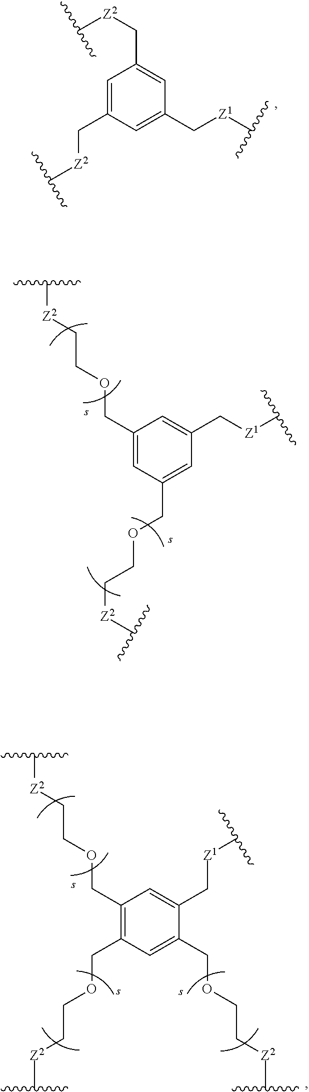

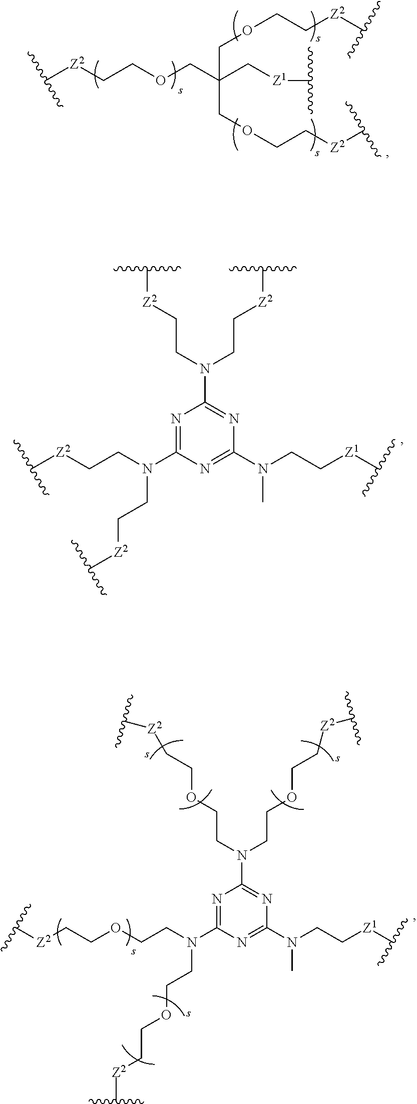

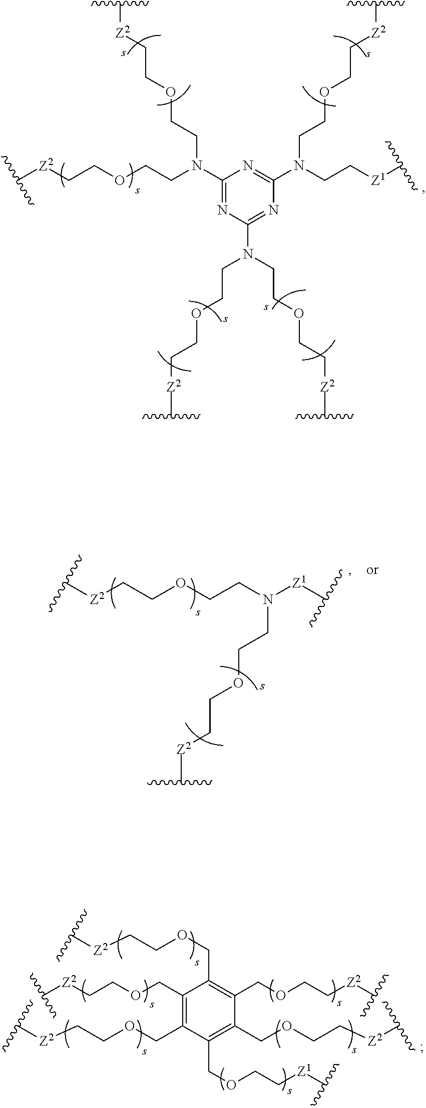

Z¹ connects Y to A and is

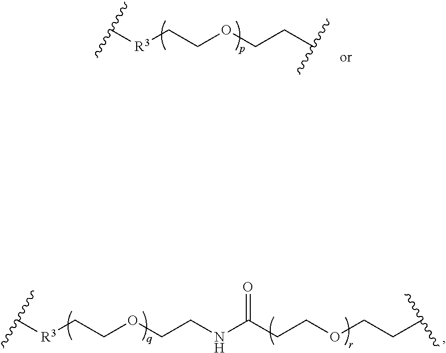

wherein
R³ is —O—, —NH—, —C(═O)—, —C(═O)N(CH₃)—, —C(═O) NH—, —NHC(═O)—, or —N(CH₃) C(═O)—,
p is an integer from 1 to 24, optionally from 4 to 24, optionally from 8 to 24, such as 8, 9, 10, 11, 12, 13, 14, 15, 16, 17, 18, 19, 20, 21, 22, 23 or 24;
q is an integer from 1 to 8, such as 1, 2, 3, 4, 5, 6, 7 or 8;
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
s is an integer from 0 to 4, such as 0, 1, 2, 3, or 4;
Z² connects Y to L and is a 1,2,3-triazole, an amide, an amide, or an urea derivative, selected from the group consisting of:

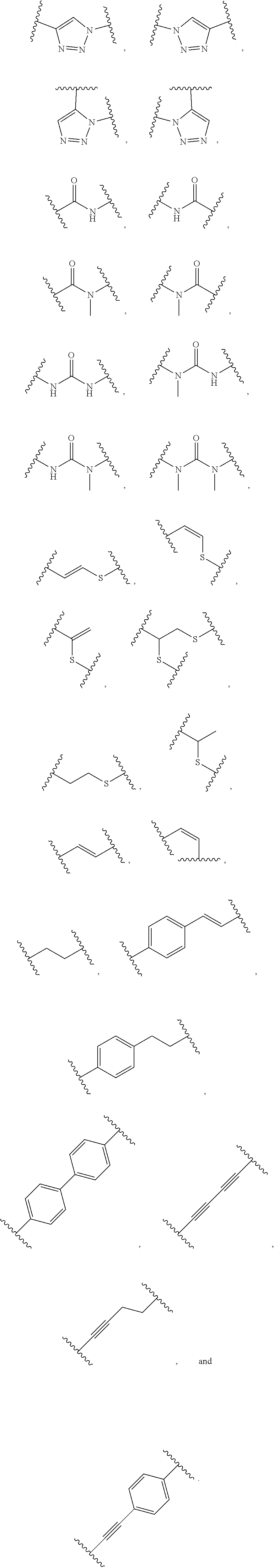

If Z⁴ is selected from the group consisting of

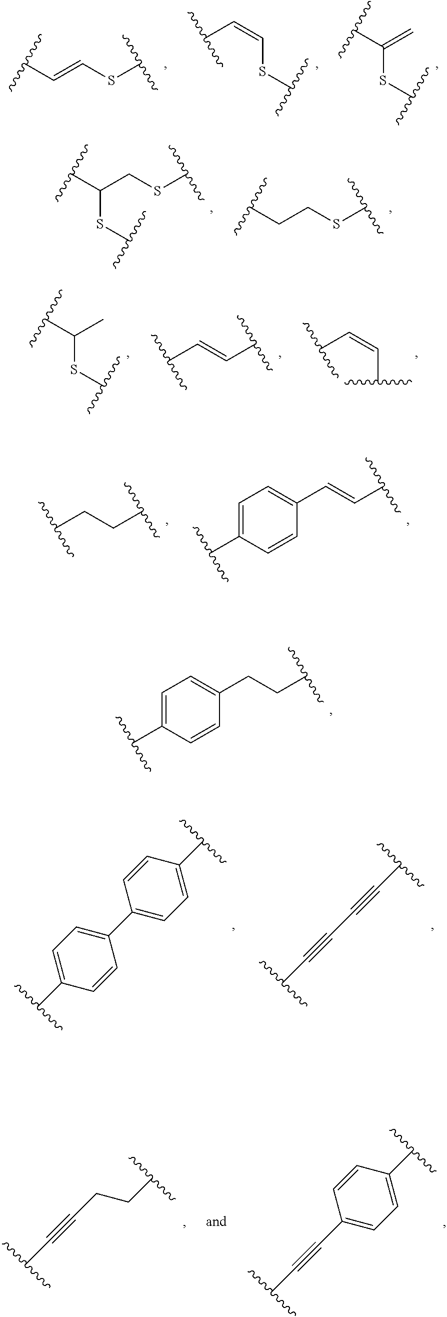

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

Depending on which of the alternative branching units Y shown above are employed, the DOL of the modular linker compound of Formula (I) can be 2, 3, 4, or 5.

Accordingly, in some embodiments of the method for synthesizing a modular linker compound of the present disclosure, the pharmaceutically active substances D and D′, i.e. the cytotoxic drug substance or substances incorporated into the modular linker compound with the DOL described above and to be delivered to the cancerous target cells, are independently selected from the group consisting of: camptothecins, optionally exatecan, SN38, and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; toll-like receptor 7 (TLR7) agonists; nicotinamide phosphoribosyltransferase (NAMPT) inhibitor (NAMPTᵢ): pyrrolobenzodiazepines (PBDs); duocarmycins; anthracyclines, optionally doxorubicin; amatoxins, optionally alpha-amanitin, beta amanitin, amanin and amaninamide; cryptophycins; phalloidins; seco-cyclopropylpyrroloindoline (seco-CPI); seco-cyclopropylbenzoindoline (seco-CBI); taxols; vinblastines; colchicines; eribulins; methotrexate (MTX); triptolide; and derivatives thereof.

In furtherance of the above, cyclodextrins are particularly suitable solubility enhancing groups SE in the context of the here-described linker compounds and their method of synthesis. For example, alpha-cyclodextrins (comprising six glucose molecules), beta-cyclodextrins and gamma-cyclodextrins, when attached to a PEG chain either directly or via a spacer Z⁴ such as:

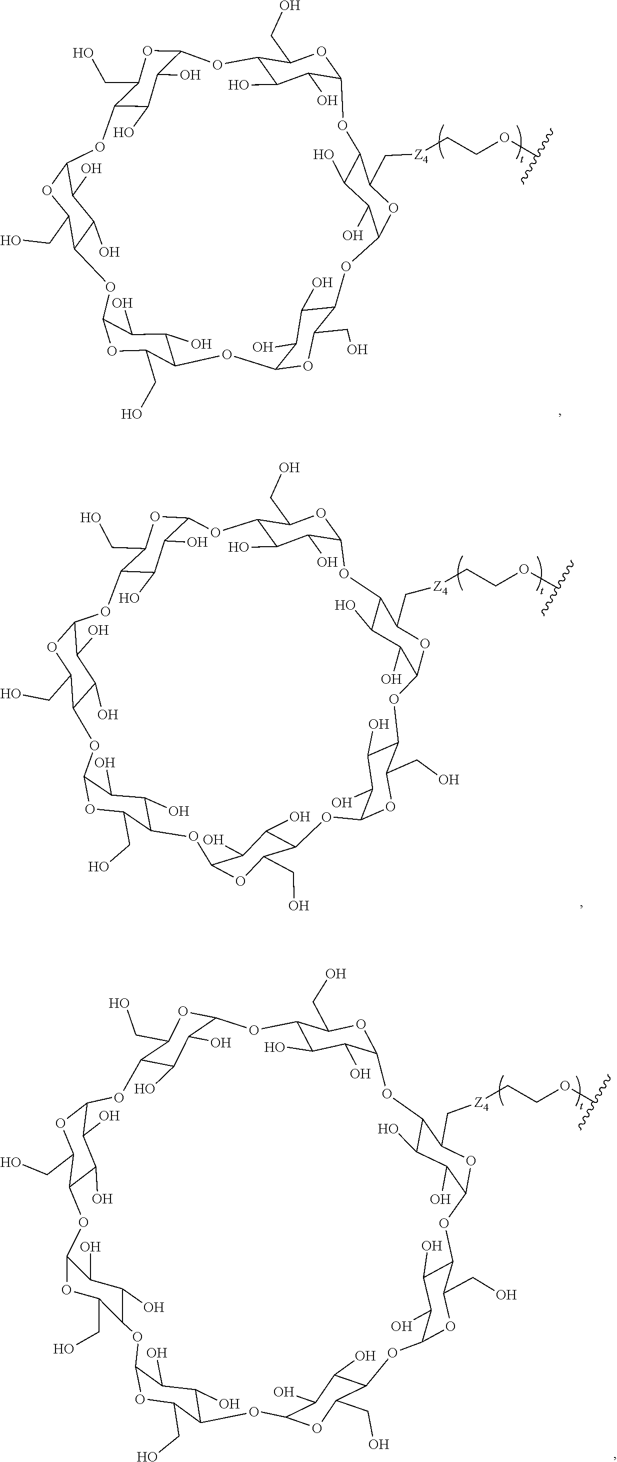

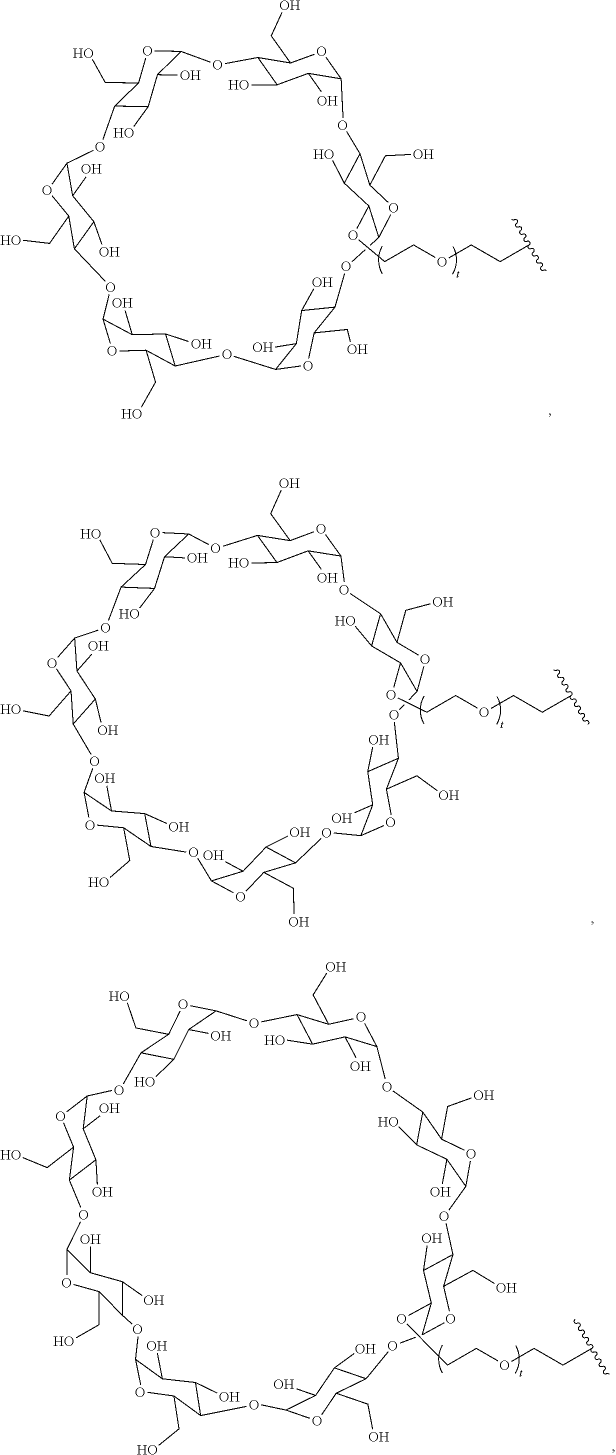

wherein Z⁴ is

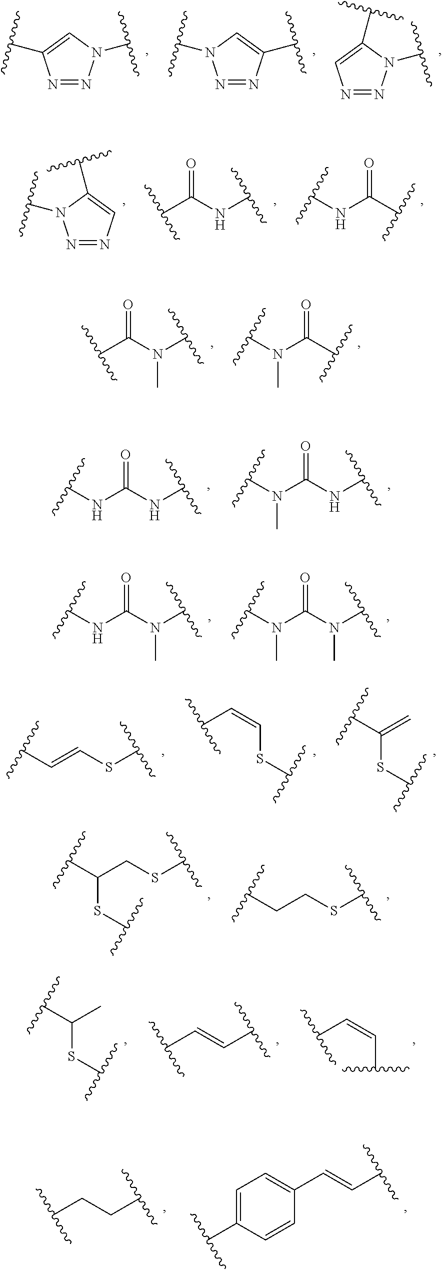

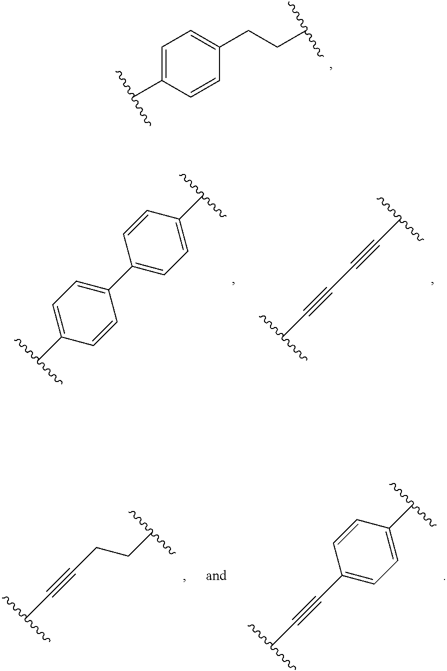 [SMILES: *CCc1ccc(*)cc1 ; *c1ccc(-c2ccc(*)cc2)cc1 ; *C#CC#C* ; *C#CCC* ; *C#Cc1ccc(*)cc1]

and wherein the PEG chain comprises between 2 and 19 PEGs, i.e, wherein t is 2 to 18, 4 to 16, 6 to 14 and most optionally 8 to 12,
constitute suitable variance of the solubility enhancing group SE.

If Z⁴ is selected from the group consisting of

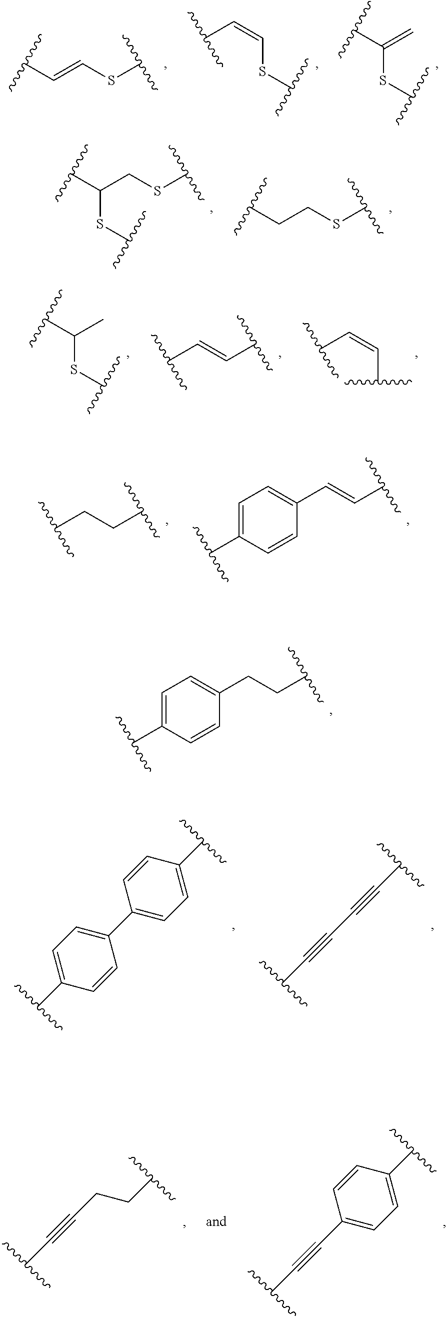

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In an embodiment where the pharmaceutical drug substance D within a

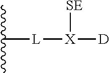

unit of a modular linker compound being synthesized in the method of the second aspect is a camptothecin, it is particularly advantageous to link a cyclodextrin enhancing group of the above illustrated types to the self-immolative spacer X via a 8 to 12 membered PEG chain, i.e. where t=8 to 12. Such PEG chain lengths favour a conformational arrangement of the linker compound that allows for the of an inclusion complex placing the lipophilic drug substance into the pocket of the cyclodextrin bring structure substantially negating the hydrophobic/lipophilic properties of the camptothecin.

Other suitable solubility enhancing groups of a modular linker compound being synthesized in the method of the second aspect include other polar groups such as
a polysarcosine

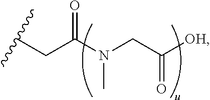

wherein u is at least 1, optionally 2, 3, 4, 5, 6, 7, 8, 9, 10, 12 or 14,
a polysarcosine amide

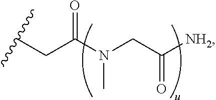

wherein u is at least 1, optionally 2, 3, 4, 5, 6, 7, 8, 9, 10, 12 or 14,
a PEG chain

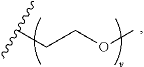

wherein v is 4 to 24 such as 4 to 22, 4 to 20, 4 to 18, 4 to 16, 4 to 14, 4 to 12, 4 to 10, or 4, 5, 6, 7, 8, 9, 10, 11 or 12,
a

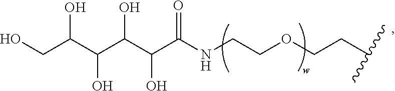

a PEG-glucamide

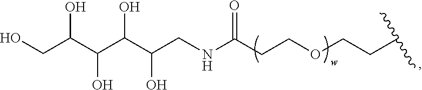

a

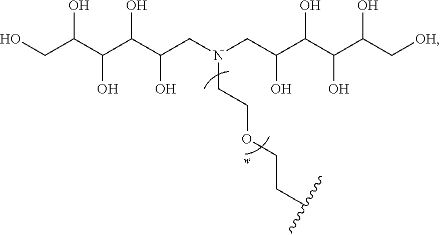

a

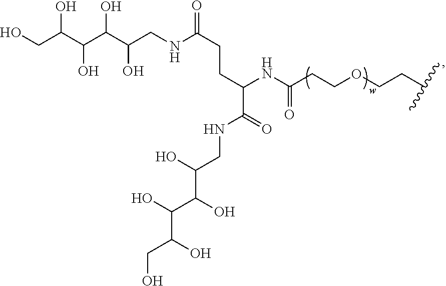

a

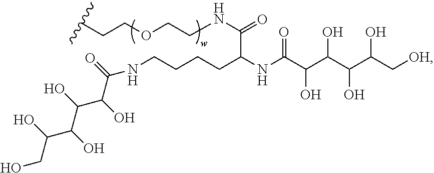

or a

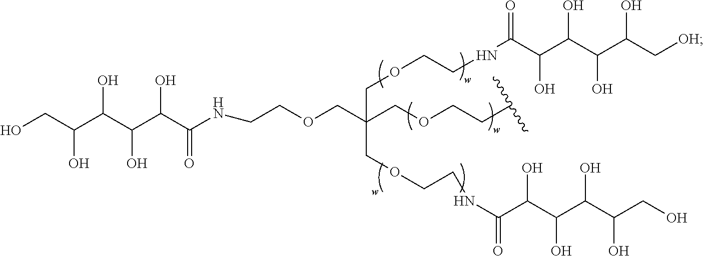

wherein w is 2 to 24 such as 2 to 22, 2 to 20, 2 to 18, 2 to 16, 2 to 14, 2 to 12, 2 to 10, or 2, 3, 4, 5, 6, 7, 8, 9, 10, 11 or 12.

As described above, the terminal reactive group A of a modular linker compound of Formula (I), i.e. of a modular linker compound being synthesized in the method of the second aspect, ensures specific and controlled conjugation of the of the linker compound to the target-binding moiety such that a stable TBDC can be generated.

In this regard, various reactive groups A have proven useful. For example, in some embodiments, the reactive group A is a thiol-reactive group such as
a 3-maleimidopropanamide group

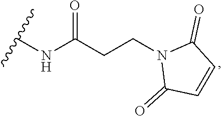 [SMILES: *NC(=O)CCN1C(=O)C=CC1=O]

a 6-maleimidohexanamide group

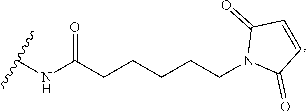 [SMILES: *NC(=O)CCCCCN1C(=O)C=CC1=O]

or
a 3-amino-2-maleimidopropanamide group

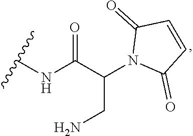 [SMILES: *NC(=O)C(CN)N1C(=O)C=CC1=O]

In alternative embodiments, reactive group A can be a 3-(2-((1,2-dimethylhydrazineyl)methyl)-1H-indol-1-yl) propanamide or 7-aza-derivative for conjugation to formylgycine moiety in a target binding moiety via the Hydrazino-Pictet-Spengler (HIPS) Ligation

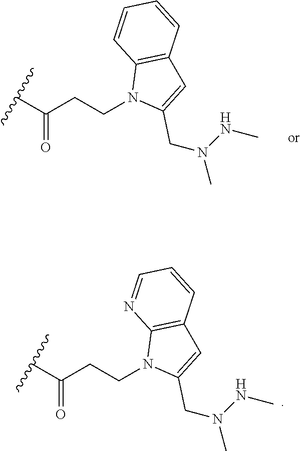 [SMILES: *C(=O)CCn1c(CN(C)NC)cc2ccccc21 ; *C(=O)CCn1c(CN(C)NC)cc2cccnc21]

In alternative embodiments, reactive group A can be a triazine compound allowing for a triazine-based conjugation of the linker compound to amine groups (primarily in lysine residues) in the target-binding moiety. In particular, reactive group A can be

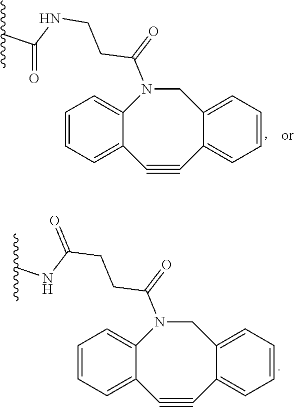 [SMILES: *C(=O)NCCC(=O)N1Cc2ccccc2C#Cc2ccccc21 ; *NC(=O)CCC(=O)N1Cc2ccccc2C#Cc2ccccc21]

In further alternative embodiments, reactive group A can be a glycine derivative, which is e.g, particularly useful for enzymatic coupling reactions using Sortase A. Exemplary glycine derivatives maybe a polyglycine comprising between 2-5 glycine residues:

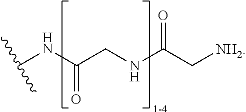

In further alternative embodiments, reactive group A can be a primary amine, e.g. lysine group

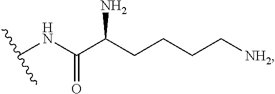 [SMILES: *NC(=O)[C@@H](N)CCCCN]

or any group comprising a primary amine (e.g. amino-modified PEG). Such conjugations may e.g. be done using transglutaminases as described in, for example, WO2015/162563 A1.

In further alternative embodiments, reactive group A can be a bicyclo[6.1.0]non-4-yn-9-ylcarbonyl, BCN, group

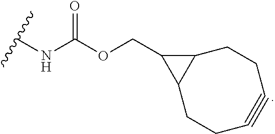 [SMILES: *NC(=O)OCC1C2CCC#CCCC21]

BCN can react with molecules containing strained alkene or alkyne bonds, such as tetrazines or other cycloalkynes, through a bioorthogonal reaction known as the inverse-electron-demand Diels-Alder (IEDDA) reaction. This reaction allows for the selective and efficient conjugation of the modular linker compound to the target-binding moiety, providing a means to create TBDCs with improved specificity and reduced off-target effects. BCN and its derivatives have gained popularity in the development of ADCs due to their bioorthogonal nature and ability to facilitate precise drug conjugation to antibodies.

Accordingly, in a specific embodiment of the third aspect, if the clogP of the pharmaceutical active compound is lower than −1, the present disclosure relates to a method for synthesizing a modular linker compound of Formula (Ia) according to the first aspect, the method comprising:
(i) Selecting a compound comprising:
   an immolative moiety,
   a cleavable peptide sequence W,
   an amine protecting group Pg¹, which is covalently linked to W,
   a pharmaceutically active substance D as an intermediate of Formula IIa:

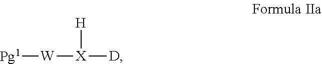

(ii) Removing the amine protecting group Pg¹ from Formula IIa to expose a primary amine;
(iii) Contacting said primary amine with a third compound comprising
   a spacer Z³,
   an amine reactive group, and
   a third complementary group Cg³
to form the intermediate of Formula IIIa:

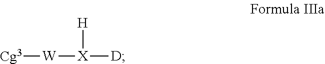

wherein L is

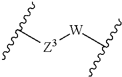

(iv) Contacting the intermediate of Formula IIIa with a branched fourth compound comprising
   a fourth complementary group Cg⁴ and
   a fifth complementary group Cg⁵,
wherein Cg³ reacts with Cg⁴ to form Z²;
(v) Contacting the product of (iv) with a fifth compound comprising
   a reactive moiety A for conjugation to a target-binding moiety and
   a sixth complementary group Cg⁶
wherein reaction of Cg⁵ and Cg⁶ forms Z¹, to form the modular linker compound of Formula Ia:

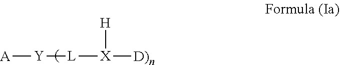

In embodiments of the method for synthesizing a modular linker compound of Formula (Ia) of the third aspect (including in the subsequently described embodiments of this method of the third aspect), (including in the subsequently described embodiments of this method of the third aspect),
Cg³ can be an alkyne group and Cg⁴ can be an azide group;
Cg³ can be an azide group and Cg⁴ can be an alkyne group;
Cg³ can be an primary or secondary amine group and Cg⁴ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group; or
Cg³ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group and Cg⁴ can be a primary or secondary amine group.

In further embodiments of the method for synthesizing a modular linker compound of Formula (Ia) of the third aspect (including in the subsequently described embodiments of this method of the second aspect),
Cg⁵ can be a primary or secondary amine group and Cg⁶ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group; or
Cg⁵ can be a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group and Cg⁶ can be a primary or secondary amine group.

In some embodiments of the method for synthesizing a modular linker compound of Formula (Ia) of the third aspect (including in the subsequently described embodiments of this method of the third aspect), the amine protecting group Pg¹ can be a carbobenzyloxy (Cbz), tert-butyloxycarbonyl (Boc), 9-fluorenylmethyloxycarbonyl (Fmoc), acetyl (Ac), benzoyl (Bz), benzyl (Bn), p-bethoxybenzyl (PMB), p-toluenesulfonyl (Ts) or a carbamate group.

As will be described in more detail in the Examples below, in some embodiments, the well-known copper-catalyzed azide-alkyne cycloaddition click chemistry reaction (CuAAC reaction) is employed highly efficiently connect various building blocks of the modular linker compound of Formula (Ia) during the steps of the method for synthesizing the modular linker compound of the second aspect.

In this method for synthesizing a modular linker compound of Formula (Ia),
X is a self-immolative spacer

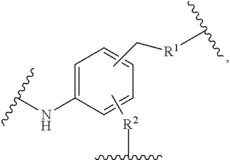

wherein:
R¹ covalently links X to D in para, meta or ortho to the amino group of X,
is a bond or

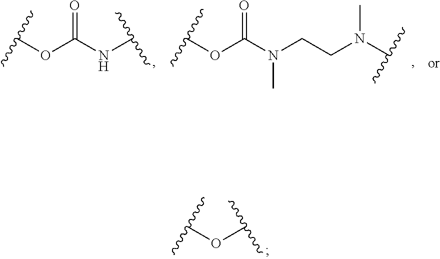

and
R² is H.

W is a cleavable and non-cleavable peptide sequence connecting Z³ and X and is selected from the group consisting of:
Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gln, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gln, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and wherein Z³ is a bond or a spacer comprising a C₁₋₂₀ alkyl chain or a 1 to 10 polyethylene glycol (PEG)₁₋₁₀ or a C₂-amide-PEG₁₋₁₀;
wherein L optionally is

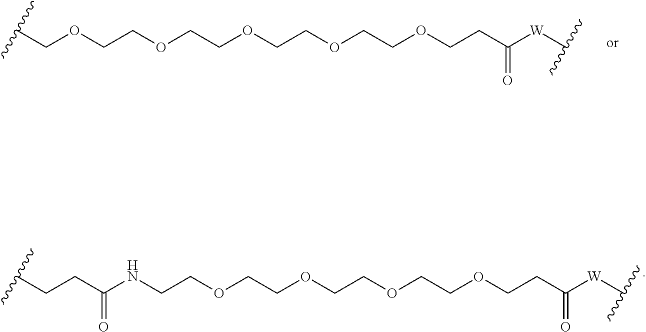

As described above, the branching unit Y of the modular linker compound of Formula (I)

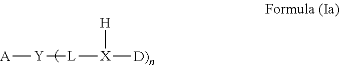

being synthesised in the method of the third aspect is:

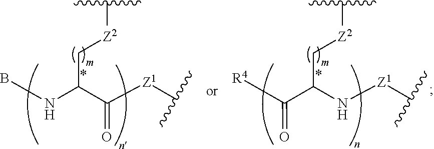

m is an integer from 1-6;
Z¹ connects Y to A and is

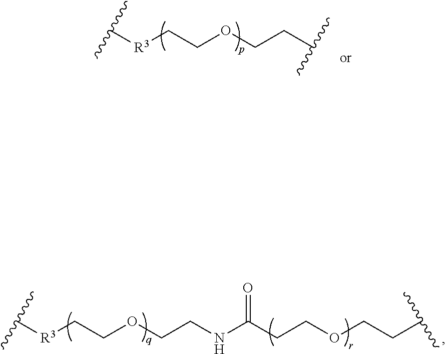

wherein
R³ is —O—, —NH—, —C(═O)—, —C(═O)N(CH₃)—, —C(═O) NH—, —NHC(═O)— or —N(CH₃) C(═O)—, and
p is an integer from 1 to 24, optionally from 4 to 24, optionally from 8 to 24, such as 8, 9, 10, 11, 12, 13, 14, 15, 16, 17, 18, 19, 20, 21, 22, 23 or 24;
q is an integer from 1 to 8, such as 1, 2, 3, 4, 5, 6, 7 or 8;
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
Z² connects Y to L and is a 1,2,3-triazole, an amide, or an urea derivative, selected from the group consisting of:

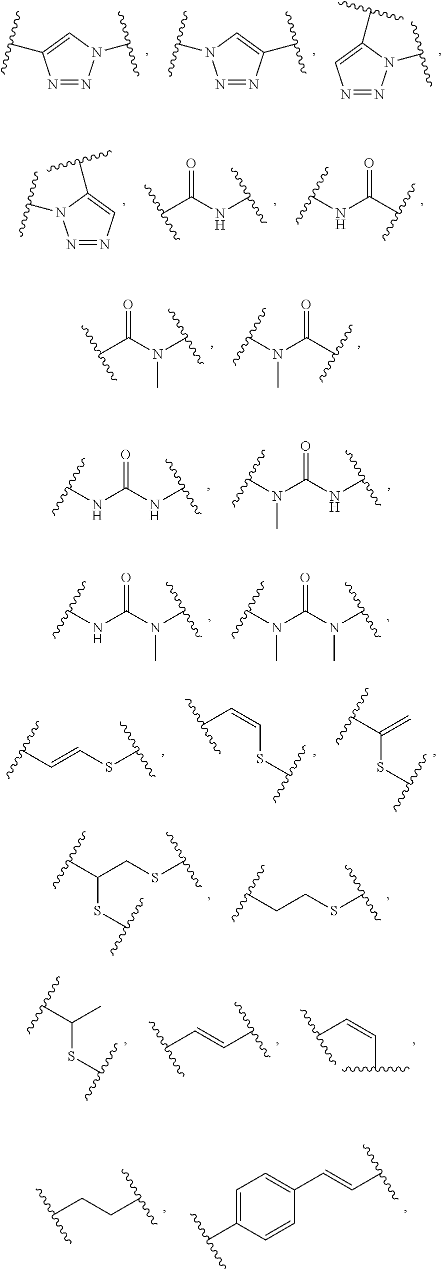

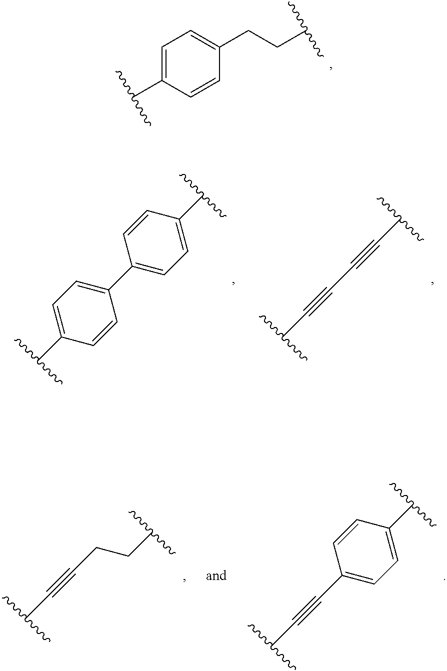 [SMILES: *CCc1ccc(*)cc1 ; *c1ccc(-c2ccc(*)cc2)cc1 ; *C#CC#C* ; *C#CCC* ; *C#Cc1ccc(*)cc1]

B is:
a cap, optionally an acyl group, and n′ is n; or

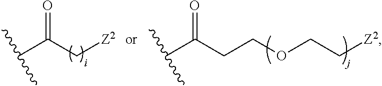

wherein
i is an integer from 1 to 20,
j is an integer from 1 to 10, and
n′ is n minus 1; or

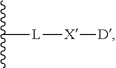

wherein
D′ is a pharmaceutically active substance different from D; and
X′ is self-immolative spacer

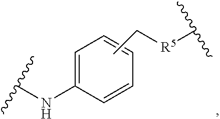

wherein:
R⁵ covalently links X′ to D′ in para or ortho to the amino group of X′ and is a bond,

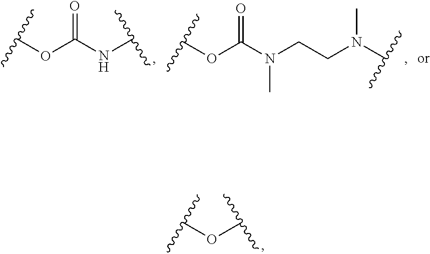

and n′ is n;
R⁴ is OH or NH₂.

If Z⁴ is selected from the group consisting of

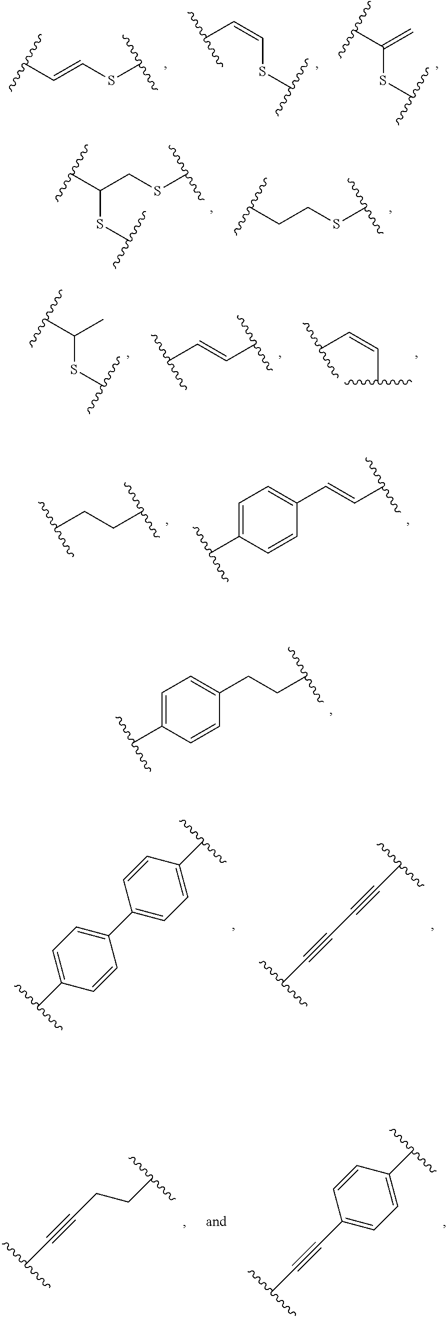

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

In embodiments where B is

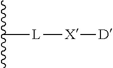

Z³ in L may further comprise a terminal carbonyl moiety, such as a dicarboxylic acid, for linkage of L to the amine group of Y.

Regardless of whether B is
a cap, optionally an acyl group, where n′ is n; or

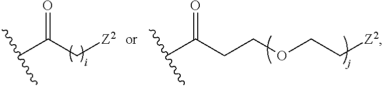

wherein
i is an integer from 1 to 20,
j is an integer from 1 to 10, and
n′ is n minus 1; or
a

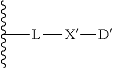

unit,
B constitutes the terminus of the final branching unit Y in multimeric core of the modular linker compound of Formula (Ia) synthesized in these embodiments of the second aspect.

As described above, the multimeric core in a modular linker compound of Formula (Ia) synthesized by the method of the third aspect can comprise n=2 to 5, optionally 2, 3, 4 or 5, branching units Y, wherein the branching unit proximal to A is connected to A via Z¹ and the final branching unit (i.e. the branching unit distal to A) comprises B. Each branching unit Y in this embodiment is indirectly linked to one molecule of a pharmaceutical drug substance D via spacer Z², spacer L and the immolative spacer X.

In alternative embodiments, branching unit Y of the modular linker compound of the Formula (Ia) being synthesized in this method of the third aspect is:

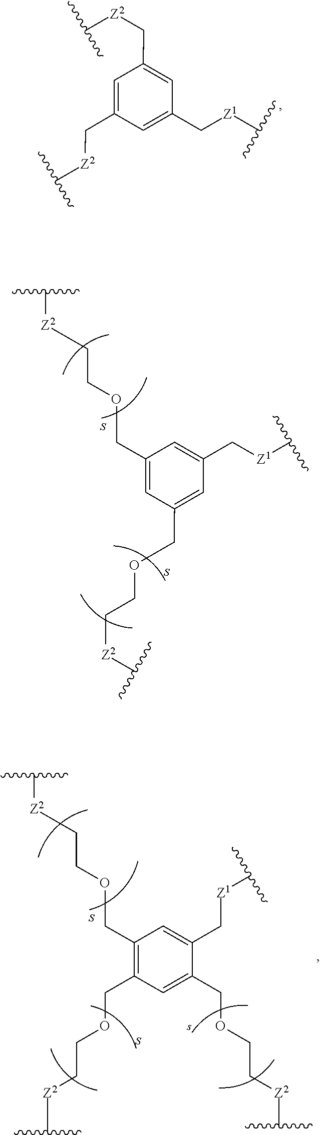

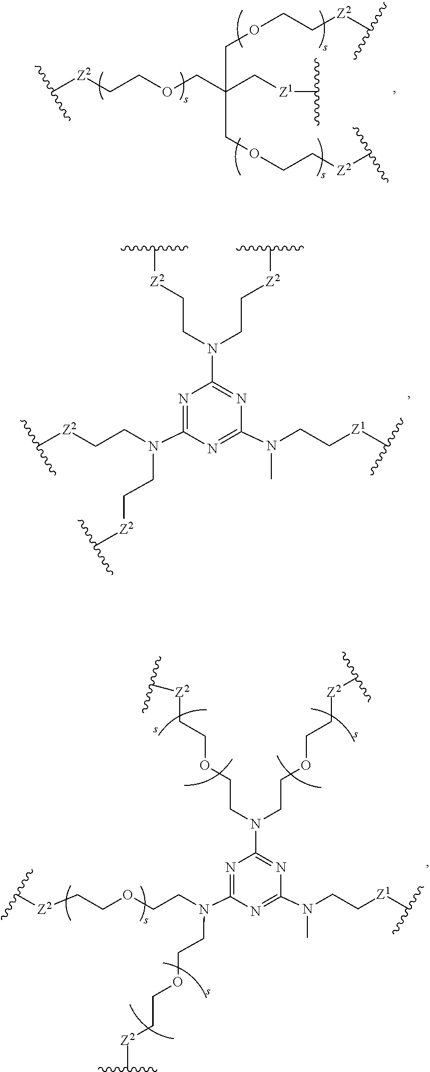

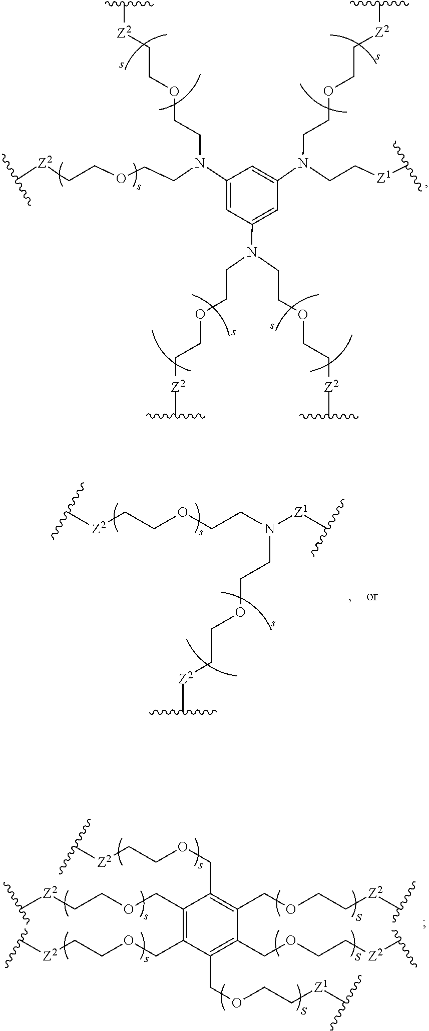

Z¹ connects Y to A and is

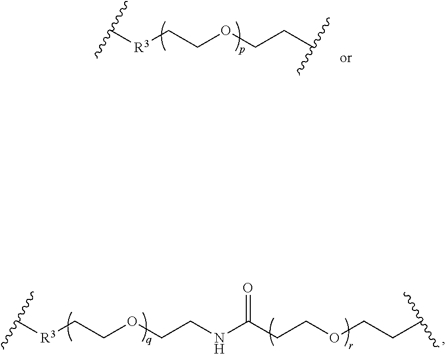

wherein
R³ is —O—, —NH—, —C(═O)—, —C(═O)N(CH₃)—, —C(═O) NH—, —NHC(═O)—, or —N(CH₃) C(═O)—,
p is an integer from 1 to 24, optionally from 4 to 24, optionally from 8 to 24, such as 8, 9, 10, 11, 12, 13, 14, 15, 16, 17, 18, 19, 20, 21, 22, 23 or 24;
q is an integer from 1 to 8, such as 1, 2, 3, 4, 5, 6, 7 or 8;
r is an integer from 1 to 24, such as 2 to 22, 2 to 20, 2 to 18, 2 to 16, 2 to 14, 2 to 12, 2 to 10, 2 to 8, 2 to 6, 2 to 4, 4 to 16, 6 to 14, 8 to 12, 2, 3, 4, 5, 6, 7 or 8;
s is an integer from 0 to 4, such as 0, 1, 2, 3, or 4;
Z² connects Y to L and is a 1,2,3-triazole, an amide, or an urea derivative, selected from the group consisting of:

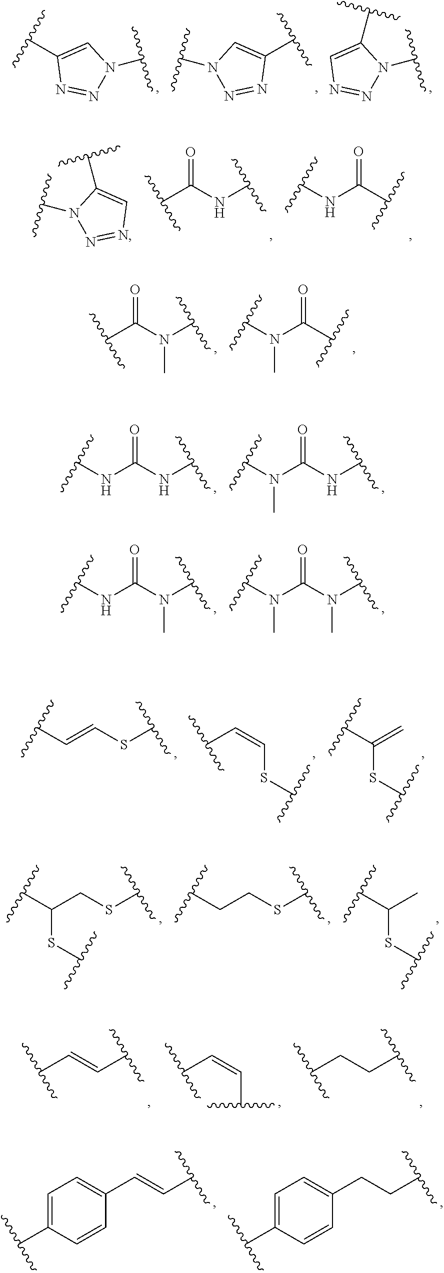

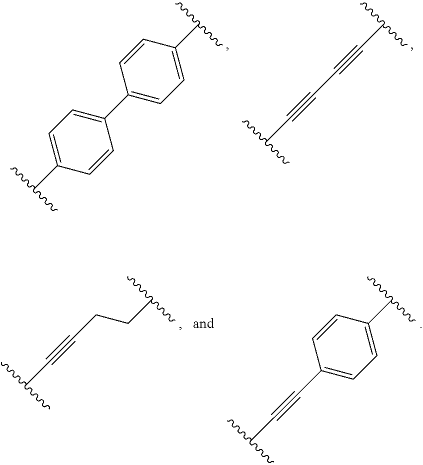

If Z⁴ is selected from the group consisting of

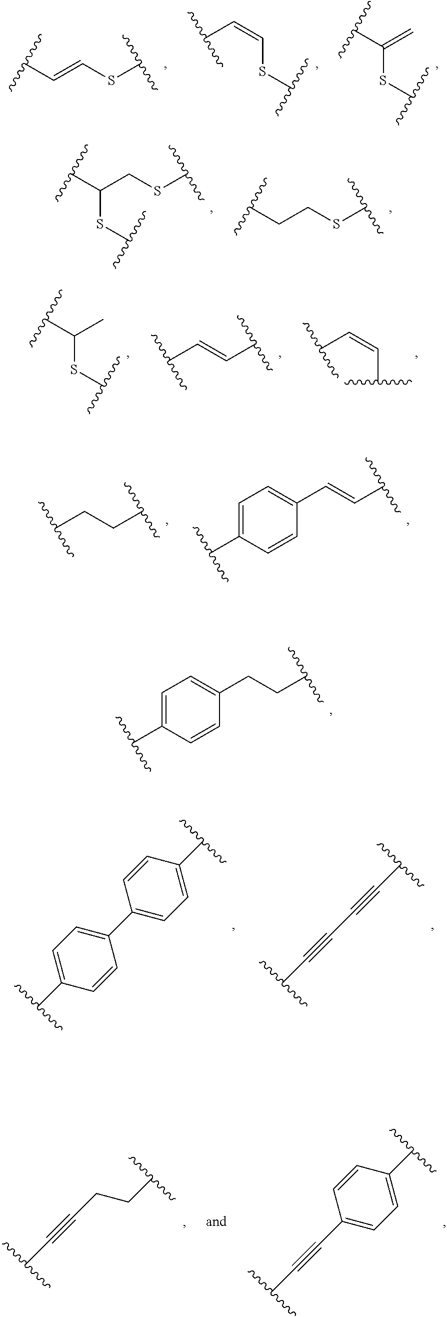

it can be attached by or used in thiol-yne/ene-reactions, cross metathesis and cross-coupling reactions as for example Heck-, Kumada-, Negishi-, Hiyama-, Stille-, Suzuki-, Sonogashira- and Glaser coupling.

Depending on which of the alternative branching units Y shown above are employed, the DOL of the modular linker compound of Formula (I) can be 2, 3, 4, or 5.

As described above, the terminal reactive group A of a modular linker compound of Formula (Ia), i.e. of a modular linker compound being synthesized in the method of the third aspect, ensures specific and controlled conjugation of the of the linker compound to the target-binding moiety such that a stable TBDC can be generated.

In this regard, various reactive groups A have proven useful. For example, in some embodiments, the reactive group A is a thiol-reactive group such as
a 3-maleimidopropanamide group

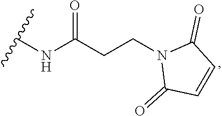 [SMILES: *NC(=O)CCN1C(=O)C=CC1=O]

a 6-maleimidohexanamide group

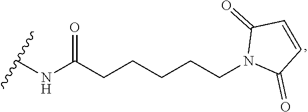 [SMILES: *NC(=O)CCCCCN1C(=O)C=CC1=O]

or
a 3-amino-2-maleimidopropanamide group

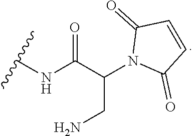 [SMILES: *NC(=O)C(CN)N1C(=O)C=CC1=O]

In alternative embodiments, reactive group A can be a 3-(2-((1,2-dimethylhydrazineyl)methyl)-1H-indol-1-yl) propanamide or 7-aza-derivative for conjugation to formylgycine moiety in a target binding moiety via the Hydrazino-Pictet-Spengler (HIPS) Ligation

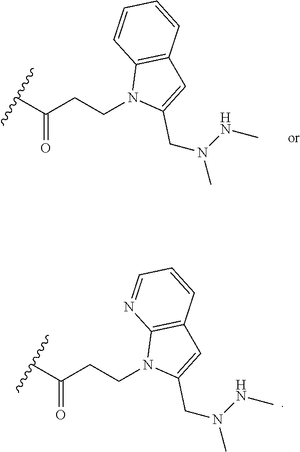 [SMILES: *C(=O)CCn1c(CN(C)NC)cc2ccccc21 ; *C(=O)CCn1c(CN(C)NC)cc2cccnc21]

In alternative embodiments, reactive group A can be a triazine compound allowing for a triazine-based conjugation of the linker compound to amine groups (primarily in lysine residues) in the target-binding moiety. In particular, reactive group A can be

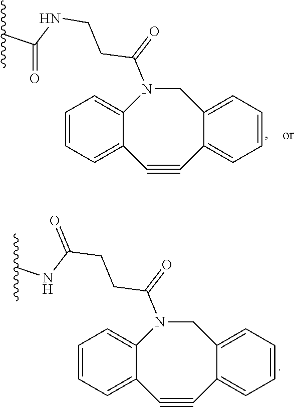 [SMILES: *C(=O)NCCC(=O)N1Cc2ccccc2C#Cc2ccccc21 ; *NC(=O)CCC(=O)N1Cc2ccccc2C#Cc2ccccc21]

In further alternative embodiments, reactive group A can be a glycine derivative, which is e.g, particularly useful for enzymatic coupling reactions using Sortase A. Exemplary glycine derivatives maybe a polyglycine comprising between 2-5 glycine residues:

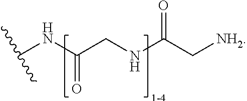

In further alternative embodiments, reactive group A can be a primary amine, e.g. lysine group

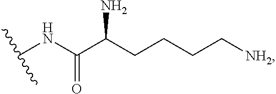 [SMILES: *NC(=O)[C@@H](N)CCCCN]

or any group comprising a primary amine (e.g. amino-modified PEG). Such conjugations may e.g. be done using transglutaminases as described in, for example, WO2015/162563 A1.

In further alternative embodiments, reactive group A can be a bicyclo[6.1.0]non-4-yn-9-ylcarbonyl, BCN, group

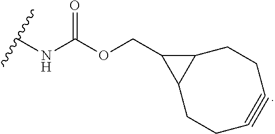 [SMILES: *NC(=O)OCC1C2CCC#CCCC21]

BCN can react with molecules containing strained alkene or alkyne bonds, such as tetrazines or other cycloalkynes, through a bioorthogonal reaction known as the inverse-electron-demand Diels-Alder (IEDDA) reaction. This reaction allows for the selective and efficient conjugation of the modular linker compound to the target-binding moiety, providing a means to create TBDCs with improved specificity and reduced off-target effects. BCN and its derivatives have gained popularity in the development of ADCs due to their bioorthogonal nature and ability to facilitate precise drug conjugation to antibodies.

Specifically preferred embodiments of the modular linker compounds of the present disclosure obtainable by the method of the third aspect are the compounds IV.1 to IV.22 shown above as well as direct end products of preferred embodiments of the method of the third aspect in the Examples further below.

In a fourth aspect, the present disclosure relates to a target-binding drug conjugate (TBDC) comprising at least one reaction product of the modular linker compound according to the first aspect and a correspondingly reactive group of a target-binding moiety, wherein the at least one reaction product is the product of a reaction between A of the modular linker compound and the correspondingly reactive group of the target-binding moiety, which covalently couples the modular linker compound to the target-binding moiety, optionally the correspondingly reactive group of the target-binding moiety is a thiol group.

Optionally, the TBDC of the fourth aspect comprises more than one of said reaction products such that more than one modular linker compound, optionally between 2 to 10 such as 2, 3, 4, 5, 6, 7, 8, 9, or 10, is coupled to said target-binding moiety.

As described above, each of the modular linker compounds of the fourth aspect comprises at least two

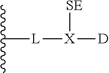

units and, thereby, each modular linker compound has at least a DOL of two (2). In embodiments of the fourth aspect, where the TBDC comprises more than one of the above reaction products, the DOL of the modular linker compound is multiplied by the number of reaction products. For example, if a TBDC comprises four (4) of said reaction products of the reactive group A of modular linker compound according to the fourth aspect with a DOL of two (2) and four (4) correspondingly reactive groups of the target-binding moiety, the TBDC will comprise four (4) such modular linker compounds and will, thereby have a DAR of 8.

In a further aspect, the present disclosure also relates to a modular linker compound, optionally, of the first aspect, when synthesized by the method of the second aspect.

In yet a further aspect, the present disclosure also relates to the use of a modular linker compound according to the first aspect in the manufacture of a target-binding drug conjugate according to the fourth aspect.

As will be appreciated generally, depending on the mechanism of action and potency of the pharmaceutically active drug substance D of the modular linker compound comprised within the TBDC and the specific therapeutic outcome sought, the modular linker compound of the present disclosure allows for the preparation of TBDCs with variable DAR scores in a relatively straightforward, i.e. combinatorial manner. This is also specifically shown in the Examples further below.

The target-binding moiety is a moiety, which specifically recognizes and binds a target sequence or epitope of an antigen. As defined further above, the target-binding moiety relies on the immunoglobulin (Ig; i.e. antibody) concept of target/epitope recognition known from molecules of the conventional Ig format (i.e. IgG, IgD, IgE, IgA and/or IgM) for the specific recognition and binding of the target sequence or target epitope of an antigen.

Accordingly, the target-binding moiety may be:
an antibody, such as a monoclonal antibody;
a functional antibody fragment or antibody derivative, such as a single-chain variable fragment (scFv), an antigen-binding fragment such as a Fab fragment, a F(ab′), fragment, a F(ab′)2 fragment or a bi- or tri-specific antibody construct;
a Diabody;
a Camelid antibody;
a Domain antibody;
a Nanobody;
a bivalent homodimer with two chains consisting of scFvs;
a shark antibody;
an antibody consisting of new-world primate framework sequences plus non-new world primate complementarity-determining regions (CDR); or
a dimerised construct comprising a constant heavy chain 3 (CH₃) domain, a variable light chain (V_{L}) and a variable heavy chain (V_{H})
a TCR.

Optionally, the target-binding moiety of the TBDC of the forth aspect specifically recognizes and binds a target sequence or epitope of a cancer-specific or cancer-associated antigen of Guanylate Cyclase C (GUCY2C), Cluster of Differentiation 33 (CD33), Cluster of Differentiation 30 (CD30), Cluster of Differentiation (CD5), Cluster of Differentiation 20 (CD20), Cluster of Differentiation 45 (CD45), Cluster of Differentiation 117 (CD117/c-Kit), Cluster of Differentiation 123 (CD123/IL3RA), Cluster of Differentiation 137 (CD137/4-1BB), Cluster of Differentiation 269 (CD269/B-cell maturation antigen, BCMA), claudin 18.2, CEA (Carcinoembryonic antigen), Epithelial Cell Adhesion Molecule (EpCam), DKK1 (Dickkopf-related protein 1), glypican-3, muscin-1 (MUC-1/CD227), Mucin 5AC (MUC5AC), Carbonic Anhydrase 9 (CAIX), C—X—C Motif Chemokine Receptor 1 (CXCR1, CD181), C—X—C Motif Chemokine Receptor 2 (CXCR2/CD182), human epidermal growth factor receptor 2 (HER2), Cluster of Differentiation 22 (CD22), Cluster of Differentiation 79b (CD79b), Nectin Cell Adhesion Molecule 4 (Nectin 4), Tumor-associated calcium signal transducer 2 (Trop-2), Cluster of Differentiation 19 (CD19), Delta-like 3 (DLL3), Transmembrane glycoprotein NMB (gpNMB), prostate-specific membrane antigen (PSMA), Hepatocyte growth factor receptor (c-Met, CD 151), Solute Carrier Family 39 Member 6 (LIV-1), Folate receptor 1 (FOLR1), Cluster of Differentiation 56 (CD56), Cluster of Differentiation 138 (CD138), Mesothelin, Carbonic anhydrase 6 (CA6), epidermal growth factor receptor (EGFR), Cluster of Differentiation 37 (CD37), Ectonucleotide pyrophosphatase/phosphodiesterase family member 3 (ENPP3).

Typically, the target-binding moiety of the TBDC is an antibody, optionally a monoclonal antibody (mAb). As is well established with respect to antibody-mediated cancer therapies, the high specificity of an mAb for single target sequence or epitope of a cancer-specific or cancer-associated antigen greatly reduces off-target effects. In addition, pharmacokinetic properties, including half-life, distribution and clearance rates can readily be established for mAbs, such that the appropriate TBDC (in this case an ADC) can be generated to ensure optimal delivery of the pharmaceutically active drug substance to the cancerous target cells. In addition, many mAbs directed at target sequences or epitopes of cancer-specific or cancer-associated antigens, are commercially available have undergone extensive clinical validation.

In preferred embodiments, the target-binding moiety according to the present disclosure is a monoclonal antibody, selected from a chimeric, humanized or human monoclonal antibody, optionally the target-binding moiety is a chimeric, humanized or human IgG1, or IgG4 monoclonal antibody.

Accordingly, in preferred embodiments of this forth aspect of the present disclosure, the target-binding moiety of the TBDC is any one of Adecatumumab (anti-EpCam), Amatuximab, (anti-mesothelin), Amivantamab (anti-EGFR), Besilesomab (anti-CEA), blinatumomab (anti-CD19), brentuximab (anti-CD30), Cantuzumab, Cibisatamab (anti-CEACAM5), Cirmtuzumab (anti-ROR1), cetuximab (anti-EGFR), Clivatuzumab (anti-MUC1), Gatipotuzumab (anti-MUC1), Coltuximab (anti-CD19), Daratumumab (anti-CD38), Duligotuzumab (anti-ERBB3), Edrecolomab (anti-EpCam), Enfortumab (anti-Nectin-4), Enoblituzumab (anti-CD276), Ensituximab (anti-MUC5AC), Epratuzumab (anti-CD22), Farletuzumab (anti-FOLR1), Igovomab (anti-CA-125), Inebilizumab (anti-CD19), Iratumumab (anti-CD30), Labetuzumab (anti-CEA), Margetuximab (anti-HER2/neu), Matuzumab (anti-EGFR), Modotuximab (anti-EGFR extracellular domain III), Naptumomab (anti-5T4), Naratuximab (anti-CD37), Necitumumab (anti-EGFR), Nimotuzumab (anti-EGFR), Obinutuzumab (anti-CD20), Ocaratuzumab (anti-CD20), Ocrelizumab (anti-CD20), Ofatumumab (anti-CD20), Otlertuzumab (anti-CD37), Ontuxizumab (anti-TEM1), Pertuzumab (anti-HER2/neu), Polatuzumab (anti-CD79b), Rituximab (anti-CD20), Rovalpituzumab (anti-DLL3), Sacituzumab (anti-TROP-2), Seribantumab (anti-ERBB3/HER3), Tafasitamab (anti-CD19), Tetulomab (anti-CD37), Timigutuzumab (anti-HER2), Trastuzumab (anti-HER2), Ublituximab (anti-CD20), or Zolbetuximab (anti-Claudin 18.2).

Additionally, in specific embodiments of the TBDC of the fourth aspect, the commercially available mAbs may have been modified by genetic engineering, for example as described in e.g. Junutula Nat Biotechnol. 2008 August; 26 (8): 925-32. Particular modifications include the incorporation of a cysteine residue in each of the heavy chains of the mAb, thereby introducing additional sulfhydryl moieties in target-binding moiety for conjugation of the modular linker compounds of the first aspect when the reactive group a of the linker compound is a thiol-reactive group. Such incorporation can readily be achieved through amino acid substitutions A118C, S239C and/or D265C (according to EU numbering system) in each of an mAb's heavy chains.

Optionally the mAb of this specific embodiment of the TBDC of the fourth aspect comprises the D265C substitution in each of its heavy chains and is conjugated to at least one modular linker compound of the first aspect via of the engineered cysteine residues of said target-binding moiety. Optionally, one modular linker compound of the first aspect is conjugated to each one of the two engineered cysteine residues at position 265 in each of the mAb's heavy chains. As described above, if the so-conjugated modular linker of the first aspect has a DOL of two (2) the TBDC has a DAR of four (4). With the variability of the modular linker compounds of the present disclosure, ADCs comprising mAbs with two engineered cysteine residues at the respective position 265 in each of the heavy chains to have a DAR between four (4) and sixteen (16). In some instances, it may be preferable for the TBDC of the fourth aspect, optionally, to also comprise the mutations L234A, L235A (numbering according to EU numbering) to reduce Fcγ receptor interaction and thereby uptake of the TBDC into an immune cell (for example, macrophages) (see e.g. Liu et al. Antibodies (2020), 9, 64.) Accordingly, in some embodiments, the TBDC of the present disclosure comprises an antibody (for example, IgG1, or IgG4 human or humanized antibody) which comprises in each of its heavy chain Fc regions the mutations L234A, L235A and D265C.

Of course, in a fifth aspect, the TBDC of the fourth aspect is specifically designed for use as a medicament, optionally for use in the treatment of cancer. Accordingly, this fourth aspect of the present disclosure also encompasses methods of treating cancer, wherein the method comprises administering the target-binding drug conjugate of the third aspect to a subject in need thereof and/or use of the target-binding drug conjugate of the third aspect in the manufacture of a medicament for the treatment of cancer.

In some embodiments, the cancer is typically characterized by the expression of at least one of the cancer-specific or cancer-associated antigens described above. Consequently, a TBDC comprising a target-binding moiety specifically directed at recognizing and binding the respective target sequence or epitope of said antigen can be used to specifically target the cells of said cancer and, ultimately, can be used in the treatment of the respective cancer.

Optionally the target sequence or epitope is present on the surface of one or more tumor cell types or tumor-associated cells in an increased concentration and/or in a different steric configuration as compared to the surface of non-tumor cells. Optionally, the antigen or epitope is present on the surface of one or more tumor or tumor stroma cell types, but not on the surface of non-tumor cells.

In a sixth aspect, the present disclosure relates to a pharmaceutical composition comprising the TBDC according to the third or fourth aspect.

In particular embodiments, the pharmaceutical composition is used in the form of a systemically administered medicament. This includes parenterals, which comprise among others injectables and infusions. Injectables are formulated either in the form of ampoules or as so called ready-for-use injectables, e.g. ready-to-use syringes or single-use syringes and aside from this in puncturable flasks for multiple withdrawal. The administration of injectables can be in the form of subcutaneous (s.c), intramuscular (i.m.), intravenous (i.v.) or intracutaneous (i.e.) application. In particular, it is possible to produce the respectively suitable injection formulations as a suspension of crystals, a solution, a nanoparticular or a colloid dispersed system like, e.g. hydrosols.

Injectable formulations can further be produced as concentrates, which can be dissolved or dispersed with aqueous isotonic diluents. The infusion can also be prepared in the form of isotonic solutions, fatty emulsions, liposomal formulations and micro-emulsions. Similar to injectables, infusion formulations can also be prepared in the form of concentrates for dilution. Injectable formulations can also be applied in the form of permanent infusions both in in-patient and ambulant therapy, e.g. by way of mini-pumps.

It is possible to add to parenteral drug formulations, for example, albumin, plasma, expander, surface-active substances, organic diluents, pH-influencing substances, complexing substances or polymeric substances, in particular as substances to influence the adsorption of the TBDCs of the present disclosure to proteins or polymers or they can also be added with the aim to reduce the adsorption of the TBDCs of the present disclosure to materials like injection instruments or packaging-materials, for example, plastic or glass.

Adjuvants and carriers added during the production of the pharmaceutical compositions of the present disclosure formulated as parenterals are optionally aqua sterilisata (sterilized water), pH value influencing substances like, e.g. organic or inorganic acids or bases as well as salts thereof, buffering substances for adjusting pH values, substances for isotonization like e.g. sodium chloride, sodium hydrogen carbonate, glucose and fructose, tensides and surfactants, respectively, and emulsifiers like, e.g. partial esters of fatty acids of polyoxyethylene sorbitans (for example, Tween®) or, e.g. fatty acid esters of polyoxyethylenes (for example, Cremophor®), fatty oils like, e.g. peanut oil, soybean oil or castor oil, synthetic esters of fatty acids like, e.g. ethyl oleate, isopropyl myristate and neutral oil (for example, Miglyol®) as well as polymeric adjuvants like, e.g. gelatine, dextran, polyvinylpyrrolidone, additives which increase the solubility of organic solvents like, e.g. propylene glycol, ethanol, N,N-dimethylacetamide, propylene glycol or complex forming substances like, e.g. citrate and urea, preservatives like, e.g. benzoic acid hydroxypropyl ester and methyl ester, benzyl alcohol, antioxidants like e.g. sodium sulfite and stabilizers like e.g. EDTA.

When formulating the pharmaceutical compositions of the present disclosure as suspensions in a preferred embodiment thickening agents to prevent the setting of the TBDCs of the present disclosure or, tensides and polyelectrolytes to assure the resuspendability of sediments and/or complex forming agents like, for example, EDTA are added. It is also possible to achieve complexes of the active ingredient with various polymers. Examples of such polymers are polyethylene glycol, polystyrene, carboxymethyl cellulose, Pluronics® or polyethylene glycol sorbit fatty acid ester. The TBDCs of the present disclosure can also be incorporated in liquid formulations in the form of inclusion compounds e.g. with cyclodextrins. In particular embodiments dispersing agents can be added as further adjuvants. For the production of lyophilisates scaffolding agents like mannite, dextran, saccharose, human albumin, lactose, PVP or varieties of gelatine can be used.

### EXAMPLES

The present disclosure is further described by the following non-limiting Examples.

### General Information

While the present disclosure has been illustrated and described in detail in the drawings and foregoing description, such illustrations and descriptions are to be considered illustrative or exemplary and not restrictive; the present disclosure is not limited to the disclosed embodiments.

All amino acid sequences disclosed herein are shown from N-terminus to C-terminus; all nucleic acid sequences disclosed herein are shown 5′->3′.

Chemicals and solvents were obtained from commercial suppliers such ABCR, Fisher Scientific, Merck Chemicals, Carl Roth, Sigma-Aldrich, VWR International, Biozol Diagnostika, TCI Deutschland, IRIS Biotech, AnalytiChem, Hycultec, Hölzel Diagnostika, Bachem, Thermo Fisher, Activate Scientific, AxisPharm, Biosynth or BLD Pharmatech and were used without further purification. The water used for reactions, workups and purifications was purified by an ELGA Purelab flex 2 pure water system. TLC was performed with silica gel 60-coated polyester sheets (Macherey-Nagel) and spots visualised by irradiation (λ=254 nm), or ninhydrin, molybdate or KMNO4 staining solutions.

### General Experimental Methods

### Analytical HPLC

Analytical RP-HPLC was carried out on a VWR-Hitachi Chromaster system equipped with a diode array detector 5430, autosampler 5260, quaternary pump 5160, column oven 6310 and a Luna® C18 (2) 5 μm 100 Å 250×4.6 mm, Kinetex® EVO-C18 5 μm 100 Å 250×4.6 mm and ACE C18-PFP 5 μm 100 Å 250×4.6 mm at 25° C. with a flowrate between 1-1.4 mL/min using linear gradient elution of acetonitrile in water. The specified parameters are stated below.

### Analytical UHPLC

Analytical RP-UHPLC was carried out on a VWR-Hitachi Chromaster Ultra Rs system equipped with a diode array detector 6430, autosampler 6270, binary pump 6170, column oven 6310 and a Kinetex® EVO-C18 1.7 μm 100 Å 100×2.1 mm with a flowrate of 0.65 mL/min using linear gradient elution of acetonitrile in water. The specified parameters are stated below.

### Method A

| Column | Phenomenex Luna C18(2), 100 Å, 250 × 4.6 mm, | | |
|---|---|---|---|
| | 5 or 10 μm | | |
| Mobile Phase | A: 0.05% TFA in water | | |
| | B: Acetonitrile | | |
| Gradient | Time [min] | A [%] | B [%] |
| | 0.0 | 95 | 5 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 210, 260 nm |
| Flow Rate | 1.4 mL/min |
| Column Temperature | 25° C. |
| Run Time | 22 min |

### Method B

| Column | Phenomenex Luna C18(2), 100 Å, 250 × 4.6 mm, | | |
|---|---|---|---|
| | 5 or 10 μm | | |
| Mobile Phase | A: 0.05% TFA in water | | |
| | B: Acetonitrile | | |
| Gradient | Time [min] | A [%] | B [%] |
| | 0.0 | 95 | 5 |
| | 0.5 | 70 | 30 |
| | 15.5 | 50 | 50 |
| | 16.0 | 0 | 100 |
| | 19.0 | 0 | 100 |
| | 19.5 | 95 | 5 |
| | 22.0 | 95 | 5 |

| | |
|---|---|
| Wavelengths | 210, 260 nm |
| Flow Rate | 1.4 mL/min |
| Column Temperature | 25° C. |
| Run Time | 22 min |

### Method C

| Column | Phenomenex Kinetex EVO-18, 100 Å, 250 × | | |
|---|---|---|---|
| | 4.6 mm, 5 μm | | |
| Mobile Phase | A: 0.05% TFA in water | | |
| | B: Acetonitrile | | |
| Gradient | Time [min] | A [%] | B [%] |
| | 0.0 | 95 | 5 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 246, 210 nm |
| Flow Rate | 1.4 mL/min |
| Column Temperature | 25° C. |
| Run Time | 22 min |

### Method D

| Column | Phenomenex Kinetex EVO-18, 100 Å, 250 × | | |
|---|---|---|---|
| | 4.6 mm, 5 μm | | |
| Mobile Phase | A: 0.05% TFA in water | | |
| | B: Acetonitrile | | |
| Gradient | Time [min] | A [%] | B [%] |
| | 0.0 | 95 | 5 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 210, 260 nm |
| Flow Rate | 1.4 mL/min |
| Column Temperature | 25° C. |
| Run Time | 22 min |

### Method E

| Column | Avantor ACE C18-PFP, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 2.0 | 70 | 30 |
| | 26.0 | 40 | 60 |
| | 26.5 | 0 | 100 |
| | 28.0 | 0 | 100 |
| | 28.5 | 95 | 5 |
| | 30.0 | 95 | 5 |

| | |
|---|---|
| Wavelengths | 210, 380 nm |
| Flow Rate | 1 mL/min |
| Column | 25° C. |
| Temperature | |
| Run Time | 30 min |

### Method F

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 2.0 | 70 | 30 |
| | 26.0 | 40 | 60 |
| | 26.5 | 0 | 100 |
| | 28.0 | 0 | 100 |
| | 28.5 | 95 | 5 |
| | 30.0 | 95 | 5 |

| | |
|---|---|
| Wavelengths | 210, 380 nm |
| Flow Rate | 1.4 mL/min |
| Column Temperature | 25° C. |
| Run Time | 30 min |

### Method G

| Column | Phenomenex Kinetex EVO-18, 100 Å, 250 × 4.6 mm, 5 μm | | |
|---|---|---|---|
| Mobile | A: Water | | |
| Phase | B: Acetonitrile | | |
| | Time [min] | A [%] | B [%] |
| Gradient | 0.0 | 95 | 5 |
| | 14.8 | 50 | 50 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 190, 210 nm |
| Flow Rate | 1.4 mL/min |
| Column Temperature | 25° C. |
| Run Time | 22 min |

### Method H

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 14.8 | 50 | 50 |
| | 15.0 | 0 | 100 |
| | 18.0 | 0 | 100 |
| | 18.5 | 95 | 5 |
| | 22.00 | 95 | 5 |

| | |
|---|---|
| Wavelengths | 210, 246 nm |
| Flow Rate | 1.4 mL/min |
| Column Temperature | 25° C. |
| Run Time | 22 min |

### Method I

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 210, 305 nm |
| Flow Rate | 1.4 mL/min |
| Column Temperature | 25° C. |
| Run Time | 22 min |

### Method J

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 26.0 | 15 | 85 |
| | 26.5 | 0 | 100 |
| | 28.0 | 0 | 100 |
| | 28.5 | 95 | 5 |
| | 30 | 95 | 5 |

| | |
|---|---|
| Wavelengths | 210, 380 nm |
| Flow Rate | 1.4 mL/min |
| Column Temperature | 25° C. |
| Run Time | 30 min |

### Method K

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 14.8 | 70 | 30 |
| | 15.0 | 0 | 100 |
| | 18.0 | 0 | 100 |
| | 18.5 | 95 | 5 |
| | 22.0 | 95 | 5 |

| | |
|---|---|
| Wavelengths | 210, 230 nm |
| Flow Rate | 1.4 mL/min |
| Column Temperature | 25° C. |
| Run Time | 22 min |

### Method L

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 25.0 | 40 | 60 |
| | 25.2 | 0 | 100 |
| | 27.8 | 0 | 100 |
| | 28.0 | 95 | 5 |
| | 30.0 | 95 | 5 |

| | |
|---|---|
| Wavelengths | 210, 260 nm |
| Flow Rate | 1.4 mL/min |
| Column Temperature | 25° C. |
| Run Time | 30 min |

### Method M

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 15.0 | 80 | 20 |
| | 15.2 | 0 | 100 |
| | 18.0 | 0 | 100 |
| | 18.5 | 95 | 5 |
| | 22.0 | 95 | 5 |

| | |
|---|---|
| Wavelengths | 205, 220 nm |
| Flow Rate | 1.4 mL/min |
| Column Temperature | 25° C. |
| Run Time | 22 min |

### Method N

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 0.5 | 80 | 20 |
| | 15.5 | 60 | 40 |
| | 16.0 | 0 | 100 |
| | 19.0 | 0 | 100 |
| | 19.5 | 95 | 5 |
| | 22.0 | 95 | 5 |

| | |
|---|---|
| Wavelengths | 210, 260 nm |
| Flow Rate | 1.4 mL/min |
| Column Temperature | 25° C. |
| Run Time | 22 min |

### Method O

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 0.5 | 80 | 20 |
| | 15.5 | 60 | 40 |
| | 16.0 | 0 | 100 |
| | 19.0 | 0 | 100 |
| | 19.5 | 95 | 5 |
| | 22.0 | 95 | 5 |

| | |
|---|---|
| Wavelengths | 210, 260 nm |
| Flow Rate | 1.4 mL/min |
| Column Temperature | 25° C. |
| Run Time | 22 min |

### Method P

| Column | Phenomenex Luna C18(2), 100 Å, 250 × 4.6 mm, 5 μm or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 30 | 70 |
| | 15.0 | 0 | 100 |
| | 18.0 | 0 | 100 |
| | 18.5 | 30 | 70 |
| | 22.0 | 30 | 70 |

| | |
|---|---|
| Wavelengths | 210, 220 nm |
| Flow Rate | 1.4 mL/min |
| Column Temperature | 25° C. |
| Run Time | 22 min |

### Method Q

| Column | Phenomenex Kinetex EVO-18, 100 Å, 100 × 2.1 mm, 1.7 μm | | |
|---|---|---|---|
| Mobile | A: 20 mM ammonium formate (pH = 4) in water | | |
| Phase | B: Acetonitrile | | |
| | Time [min] | A [%] | B [%] |
| Gradient | 0.0 | 95 | 5 |
| | 3.2 | 0 | 100 |
| | 3.95 | 0 | 100 |
| | 4.15 | 95 | 5 |
| | 5 | 95 | 5 |

| | |
|---|---|
| Wavelengths | 210, 260 nm |
| Flow Rate | 0.65 mL/min |
| Column Temperature | 25° C. |
| Run Time | 5 min |

### Method R

| Column | Phenomenex Kinetex EVO-18, 100 Å, 100 × 2.1 mm, 1.7 μm | | |
|---|---|---|---|
| Mobile | A: 20 mM ammonium formate (pH = 4) in water | | |
| Phase | B: Acetonitrile | | |
| | Time [min] | A [%] | B [%] |
| Gradient | 0.0 | 95 | 5 |
| | 6.0 | 0 | 100 |
| | 6.95 | 0 | 100 |
| | 7.15 | 95 | 5 |
| | 8 | 95 | 5 |

| | |
|---|---|
| Wavelengths | 210, 260 nm |
| Flow Rate | 0.65 mL/min |
| Column Temperature | 25° C. |
| Run Time | 8 min |

### Method S

| Column | Phenomenex Kinetex EVO-18, 100 Å, 100 × 2.1 mm, 1.7 μm | | |
|---|---|---|---|
| Mobile | A: 20 mM ammonium formate (pH = 4) in water | | |
| Phase | B: Acetonitrile | | |
| | Time [min] | A [%] | B [%] |
| Gradient | 0.0 | 95 | 5 |
| | 6.0 | 15 | 85 |
| | 6.2 | 0 | 100 |
| | 6.95 | 95 | 100 |
| | 7.15 | 95 | 5 |
| | 8.0 | 95 | 5 |

| | |
|---|---|
| Wavelengths | 210, 260 nm |
| Flow Rate | 0.65 mL/min |
| Column Temperature | 25° C. |
| Run Time | 8 min |

### Method T

| Column | Phenomenex Kinetex EVO-18, 100 Å, 100 × 2.1 mm, 1.7 μm | | |
|---|---|---|---|
| Mobile | A: 20 mM ammonium formate (pH = 4) in water | | |
| Phase | B: Acetonitrile | | |
| | Time [min] | A [%] | B [%] |
| Gradient | 0.0 | 95 | 5 |
| | 0.09 | 80 | 20 |
| | 4.6 | 60 | 40 |
| | 5 | 0 | 100 |
| | 6 | 0 | 100 |
| | 6.5 | 95 | 5 |
| | 8 | 95 | 5 |

| | |
|---|---|
| Wavelengths | 210, 260 nm |
| Flow Rate | 0.65 mL/min |
| Column Temperature | 25° C. |
| Run Time | 8 min |

### Method U

| Column | Phenomenex Kinetex EVO-18, 100 Å, 100 × 2.1 mm, 1.7 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 0.2 | 70 | 30 |
| | 6.0 | 40 | 60 |
| | 6.2 | 0 | 100 |
| | 6.95 | 0 | 100 |
| | 7.15 | 95 | 5 |
| | 8 | 95 | 5 |

| | |
|---|---|
| Wavelengths | 210, 260 nm |
| Flow Rate | 0.65 mL/min |
| Column Temperature | 25° C. |
| Run Time | 8 min |

### Method V

| Column | Phenomenex Kinetex EVO-18, 100 Å, 100 × 2.1 mm, 1.7 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 1.7 | 0 | 100 |
| | 2.15 | 0 | 100 |
| | 2.35 | 95 | 5 |
| | 3.0 | 95 | 5 |

| | |
|---|---|
| Wavelengths | 210, 380 nm |
| Flow Rate | 0.65 mL/min |
| Column Temperature | 25° C. |
| Run Time | 3 min |

### Method W

| Column | Phenomenex Kinetex EVO-18, 100 Å, 100 × 2.1 mm, 1.7 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 3.2 | 15 | 85 |
| | 3.4 | 0 | 100 |
| | 3.95 | 95 | 100 |
| | 4.15 | 95 | 5 |
| | 5.0 | 95 | 5 |

| | |
|---|---|
| Wavelengths | 210, 380 nm |
| Flow Rate | 0.65 mL/min |
| Column Temperature | 25° C. |
| Run Time | 5 min |

### Method X

| Column | Interchim Uptisphere Strategy, 100 Å, 150 × 4.6 mm, 3 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 9.1 | 0 | 100 |
| | 12.6 | 0 | 100 |
| | 13.0 | 95 | 5 |
| | 15.4 | 95 | 5 |

| | |
|---|---|
| Wavelengths | 210, 264 nm |
| Flow Rate | 1 mL/min |
| Column Temperature | 25° C. |
| Run Time | 15.4 min |

### Method Y

| Column | Interchim Uptisphere Strategy, 100 Å, 150 × 4.6 mm, 3 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 10.5 | 60 | 40 |
| | 10.6 | 0 | 100 |
| | 12.6 | 0 | 100 |
| | 13.0 | 95 | 5 |
| | 15.4 | 95 | 5 |

| | |
|---|---|
| Wavelengths | 210, 264 nm |
| Flow Rate | 1 mL/min |
| Column Temperature | 25° C. |
| Run Time | 15.4 min |

### Method Z

| Column | Interchim Uptisphere Strategy, 100 Å, 150 × 4.6 mm, 3 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 4.0 | 75 | 25 |
| | 9.5 | 43 | 57 |
| | 10.0 | 0 | 100 |
| | 13.0 | 0 | 100 |
| | 13.4 | 95 | 5 |
| | 15.0 | 95 | 5 |

| | |
|---|---|
| Wavelengths | 210, 264 nm |
| Flow Rate | 1 mL/min |
| Column Temperature | 25° C. |
| Run Time | 15 min |

### Method AA

| Column | Interchim Uptisphere Strategy, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 14.0 | 18.8 | 81.2 |
| | 14.5 | 0 | 100 |
| | 17.0 | 0 | 100 |
| | 17.5 | 95 | 5 |
| | 20.0 | 95 | 5 |

| | |
|---|---|
| Wavelengths | 210, 264 nm |
| Flow Rate | 1.4 mL/min |
| Column Temperature | 25° C. |
| Run Time | 20 min |

### Method AB

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 15.0 | 40 | 60 |
| | 15.2 | 0 | 100 |
| | 18.0 | 0 | 100 |
| | 18.5 | 95 | 5 |
| | 22.0 | 95 | 5 |

| | |
|---|---|
| Wavelengths | 210, 240 nm |
| Flow Rate | 1.4 mL/min |
| Column Temperature | 25° C. |
| Run Time | 22 min |

### Method AC

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 15.0 | 50 | 50 |
| | 15.2 | 0 | 100 |
| | 18.0 | 0 | 100 |
| | 18.5 | 95 | 5 |
| | 22.0 | 95 | 5 |

| | |
|---|---|
| Wavelengths | 210, 240 nm |
| Flow Rate | 1.4 mL/min |
| Column Temperature | 25° C. |
| Run Time | 22 min |

### Method AD

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 5.0 | 80 | 20 |
| | 26.0 | 60 | 40 |
| | 26.5 | 0 | 100 |
| | 28.0 | 0 | 100 |
| | 28.5 | 95 | 5 |
| | 30.0 | 95 | 5 |

| | |
|---|---|
| Wavelengths | 210, 380 nm |
| Flow Rate | 1.4 mL/min |
| Column Temperature | 25° C. |
| Run Time | 30 min |

### Method AE

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 0.5 | 80 | 20 |
| | 20.5 | 53.3 | 47.7 |
| | 21.0 | 0 | 100 |
| | 24.0 | 0 | 100 |
| | 24.5 | 95 | 5 |
| | 27.0 | 95 | 5 |

| | |
|---|---|
| Wavelengths | 210, 250 nm |
| Flow Rate | 1.4 mL/min |
| Column Temperature | 25° C. |
| Run Time | 27 min |

### Method AF

| Column | Phenomenex Luna C18(2), 100 Å, 250 × 4.6 mm, 5 or 10 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 25.0 | 40 | 60 |
| | 25.5 | 0 | 100 |
| | 27.5 | 0 | 100 |
| | 28.0 | 95 | 5 |
| | 30.0 | 95 | 5 |

| | |
|---|---|
| Wavelengths | 210, 290, 305 nm |
| Flow Rate | 1.4 mL/min |
| Column Temperature | 25° C. |
| Run Time | 30 min |

### Method AG

| Column | Phenomenex Kinetex EVO-18, 100 Å, 250 × 4.6 mm, 5 μm | | |
|---|---|---|---|
| Mobile | A: 0.05% TFA in water | | |
| Phase | B: Acetonitrile | | |
| | Time [min] | A [%] | B [%] |
| Gradient | 0.0 | 95 | 5 |
| | 0.5 | 80 | 20 |
| | 26 | 53.3 | 40 |
| | 26.5 | 0 | 100 |
| | 28 | 0 | 100 |
| | 28.5 | 95 | 5 |
| | 30.0 | 95 | 5 |

| | |
|---|---|
| Wavelengths | 210, 264. 380 nm |
| Flow Rate | 1.4 mL/min |
| Column Temperature | 25° C. |
| Run Time | 30 min |

### NMR Spectroscopy

¹H and spectra were recorded at 500 MHz at room temperature and were referenced to trimethylsilane (TMS). Chemical shifts (8) are reported in parts per million (ppm) from low field to high field. Coupling constants (J) are reported in Hertz (Hz) and abbreviations indicating multiplicity are used as follows: s=singlet, d=doublet, t=triplet, dd=doublet of doublet, m=multiplet.

### Mass Spectrometry (MS)

MS measurements were carried out on an Advion expression® CMS. Samples were submitted by direct injection with a syringe. For ionization, an electrospray ionization source (ESI) or atmospheric pressure ionization source (APCI) was used. Analyses were performed in positive ion mode. For ESI, the capillary temperature was set at 250° C. The source gas temperatures were set at 250 and 300° C., respectively with a flow of 4 and 5 L/min, respectively. Nebulizing gas flow was set to 0.5 L/min. The electrospray voltage was set at 3.5 kV. For APCI, the capillary temperature was set at 250° C., source gas temperature at 350° C. and APCI corona discharge at 5 μA was used.

### Preparative RP-HPLC Purification

Preparative RP-HPLC purification was carried out on an Agilent 1260 Infinity II system equipped with a binary pump, multisampler, VWD, fraction collector and column organizer. As columns, Phenomenx Luna® C18 (2) 10 μm 100 Å 250×21.2 mm, Kinetex® EVO-C18 5 μm 100 Å 250×21.2 mm and ACE C18-PFP 5 μm 100 Å 250×21.2 mm with flow rates between 24-30 mL/min using linear gradient elution of acetonitrile in water (+0.05% TFA). In case acid (e.g. TFA) was used as solvent additive during purification, compounds comprising basic groups such as amines were obtained as the corresponding salts (e.g. TFA salt).

### Flash Chromatography

Purification by flash chromatography on silica was performed using a Teledyne ISCO CombiFlash NextGen 300+ equipped with UV and ELSD detection and RediSep® single-use normal-phase silica columns with mesh between 230 and 400.

### General Procedure 1 (GP1): CuAAC Click Reaction for Attachment of Solubility Enhancer

To a stirred solution of azide (0.9-1.5 equiv.) and alkyne (1 equiv.) in DMF was added DMF:water (5:1 or 10:1) containing tris[(1-benzyl-1H-1,2,3-triazol-4-yl)methyl]amin) (TBTA, 0.125-0.625 equiv.) or tris(3-hydroxypropyltriazolylmethyl)amine (THPTA, 0.125-2 equiv.), copper (II) sulfate pentahydrate (0.1-1 equiv.) and Na-ascorbate (0.25-3.0 equiv.). The resulting reaction mixture was allowed to stir at room temperature until completion. Upon completion, the reaction mixture was purified directly without any pretreatment by prep HPLC to yield the product after lyophilization.

### General Procedure 2 (GP2): CuAAC Click Reaction on Polyazide Scaffold

To a stirred solution of polyazide (1 equiv.) and alkyne (1.05-1.25 equiv. per azide moiety) in DMF was added water containing Tris(3-hydroxypropyltriazolylmethyl)amine (THPTA, 0.63-1.35 equiv. per azide moiety), copper (II) sulfate pentahydrate (0.525-0.65 equiv. per azide moiety) and Na-ascorbate (1-2.15 equiv. per azide moiety). The resulting ratio of DMF/water was 5:1 or 10:1. The resulting reaction mixture was allowed to stir at room temperature until completion. Upon completion, the reaction mixture was purified directly without any pretreatment by prep HPLC to yield the multimeric linker-payload after lyophilization.

### General Procedure 3 (GP3): Attachment of Intermediate 3.1 or 3.2

Fmoc-protected linker-payload was treated with diethylamine (Et₂N, 20-24 equiv.) in DMF at room temperature and the reaction progress followed by RP-HPLC. The solvent was removed in vacuo and the residue co-evaporated once with DMF. Then, the residue was dissolved in DMF under argon and a solution of intermediate 3.1 or 3.2 (1.1-1.5 equiv.) in DMF was added followed by N-ethyldiisopropylamine (DIPEA, 1.4-3 equiv.). The solution was stirred at room temperature and the progress followed by RP-HPLC. Upon completion, the solvent was evaporated, and the crude product purified by RP-HPLC.

### General Procedure 4 (GP4): Attachment of Maleimide

To a stirred solution of multimeric linker-payload in dry DMF was added Mal-PEG (4)-NHS ester (1-4 equiv.) and DIPEA (1-4 equiv.). The reaction mixture was stirred at room temperature for 30 min. Upon completion, the reaction mixture was purified directly without any pretreatment by prep HPLC to yield the final compound after lyophilization.

### General Procedure 5 (GP5): Attachment of PEG-Azide to Mono-6-Amino-6-Deoxy-Cyclodextrin

Azido-PEG-NHS ester (1 equiv.) was dissolved in water or DMF and a solution of mono-6-amino-6-deoxy-cyclodextrin (1 equiv.) in water or DMF was added. In case DMF was used as solvent, DIPEA (1.5-2 equiv.) was added if necessary. The resulting reaction mixture was allowed to stir at room temperature until completion and monitored by RP-HPLC. If necessary, another portion of mono-6-amino-6-deoxy-cyclodextrin (0.2 equiv.) was added. Upon completion, the reaction mixture was freeze dried and used as crude without further purification in case water was used as solvent. In case DMF was used as solvent, the product was isolated by precipitation from MTBE. In case of mono-6-amino-6-deoxy-beta-cyclodextrin use, commercially available mono-6-amino-6-deoxy-beta-cyclodextrin was employed. In case of mono-6-amino-6-deoxy-gamma-cyclodextrin use, the material was synthesized from gamma-cyclodextrin according to Tang, W., Ng, S.-C. Nat. Protoc. 2008, 3, 691-697.

### ADC Generation—Conjugation of Linker Payloads to Antibodies

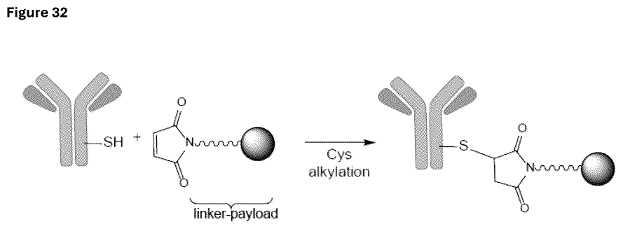

Antibodies were conjugated to the drug-carrying linker conjugates by means of the so-called Thiomab technology. In this approach, the conjugation takes place by coupling a terminal thiol-reactive moiety such as a maleimide residue of the drug-carrying linker to the free SH group of a genetically-engineered cysteine residue in the Fc region of the antibody, as shown in FIG. 32. The principles of this conjugation method are disclosed in Junutula et al. Nat Biotechnol. 2008 August; 26 (8): 925-32, the content of which is incorporated herein by reference.

The anti-GUCY2C antibodies used in the present experiments comprise a D265C substitution in both Fc domains, in order to provide a cysteine residue that has a free SH group. The respective technology is disclosed in WO2016/142049 A1, the content of which is incorporated herein by reference.

### Example 1—Synthesis of Modular Linker Compounds

### Example 1.1—Syntheses of Linker Intermediates

### Step 1: Synthesis of 4-nitro-2-((prop-2-yn-1-yloxy)methyl)benzoic acid

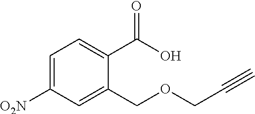 [SMILES: C#CCOCc1cc([N+](=O)[O-])ccc1C(=O)O]

To a solution of Methyl-2-(bromomethyl)-4-nitrobenzoate (CAS-Nr. 133446-99-8, 2 g, 7.30 mmol, 1 equiv.) in 10 mL acetonitrile were added prop-2-yn-ol (2.16 mL, 36.5 mmol, 5 equiv.) and Cs₂CO₃ (5.96 g, 18.3 mmol, 2.5 equiv.) at room temperature. The mixture was stirred at 80° C. for 3 h and the progress monitored by RP-HPLC. The reaction mixture was filtered. The filtrate was diluted with ethyl acetate (20 mL) and washed with water (10 mL) and extracted with NaHCO₃ (2×10 mL). For precipitation, the combined aqueous layers were acidified to pH 2 by using 2 M HCl (50 mL). After filtration, the solid obtained was taken up in 1,4-dioxane and lyophilized overnight to yield 4-nitro-2-((prop-2-yn-1-yloxy)methyl)benzoic acid as pale red solid (1.44 g, 83%). ¹H NMR (500 MHZ, DMSO) δ 8.36 (d, J=2.4 Hz, 1H), 8.22 (dd, J=8.6, 2.5 Hz, 1H), 8.09 (d, J=8.5 Hz, 1H), 4.94 (s, 2H), 4.36 (d, J=2.4 Hz, 2H), 3.53 (t, J=2.4 Hz, 1H). HPLC (method A): t_{R}=11.3 min.

### Step 2: Synthesis of Methyl-4-nitro-2-((prop-2-yn-1-yloxy)methyl)benzoate

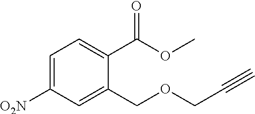 [SMILES: C#CCOCc1cc([N+](=O)[O-])ccc1C(=O)OC]

To a solution of 4-nitro-2-((prop-2-yn-1-yloxy)methyl)benzoic acid (1.44 g, 6.12 mmol, 1 equiv.) in 30 mL dry methanol was added SOCl₂ (3.56 mL, 49 mmol, 8 equiv.) at 0° C. The mixture was stirred at 70° C. for 3.5 h and the progress monitored by RP-HPLC. Upon completion, the solvent and the volatiles were removed under reduced pressure. The obtained residue was taken up in 1,4-dioxane and lyophilized overnight to yield methyl-4-nitro-2-((prop-2-yn-1-yloxy)methyl)benzoate (1.52 g, 99%) as pale brown solid. ¹H NMR (500 MHZ, DMSO) δ 8.37 (dd, J=2.3, 1.1 Hz, 1H), 8.28-8.22 (m, 1H), 8.08 (d, J=8.5 Hz, 1H), 4.92 (d, J=0.8 Hz, 2H), 4.34 (d, J=2.4 Hz, 2H), 3.90 (s, 3H), 3.49 (t, J=2.4 Hz, 1H). HPLC (method A): t_{R}=13.1 min.

### Step 3: Synthesis of methyl 4-amino-2-((prop-2-yn-1-yloxy)methyl)benzoate

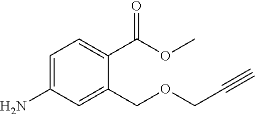 [SMILES: C#CCOCc1cc(N)ccc1C(=O)OC]

To a stirred suspension of methyl-4-nitro-2-((prop-2-yn-1-yloxy)methyl)benzoate (1.52 g, 6.1 mmol, 1 equiv.) in a mixture of EtOH (15 mL) and water (7.5 mL) was added iron powder (2.73 g, 48.8 mmol, 8 equiv.) and NH₄Cl (2.61 g, 48.8 mmol, 8 equiv.) at room temperature. The resulting mixture was allowed to stir at 80° C. for 4 h and the progress monitored by RP-HPLC. After total consumption of the starting material, the reaction was cooled to rt and filtered through celite and washed with ethyl acetate (15 mL). The filtrate was washed with sat. sodium bicarbonate solution, water and brine (each 15 mL). The aqueous layers were extracted with ethyl acetate (15) mL. The combined organic layers were dried over MgSO₄. After filtration the solvent was removed under reduced pressure to obtain 4-amino-2-((prop-2-yn-1-yloxy)methyl)benzoate as red solid (1.29 g, 96%) after drying in vacuo. ¹H NMR (500 MHZ, CDCl₃) δ 7.87 (d, J=8.5 Hz, 1H), 6.99 (dt, J=2.2, 1.0 Hz, 1H), 6.59 (dd, J=8.5, 2.5 Hz, 1H), 5.00 (s, 2H), 4.31 (d, J=2.4 Hz, 2H), 3.84 (s, 3H), 2.48 (t, J=2.4 Hz, 1H). HPLC (method A): t_{R}=10.5 min.

### Step 4: Synthesis of (4-amino-2-((prop-2-yn-1-yloxy)methyl)phenyl) methanol

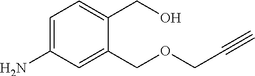

To a cooled solution (0° C.) of dry THF (13 mL) was added LiAlH₄ (1 M in THF, 12.01 mmol, 12.01 mL, 2.5 equiv.) slowly. A solution of 4-amino-2-((prop-2-yn-1-yloxy)methyl)benzoate (1.06 g, 4.8 mmol, 1 equiv.) in dry THF (10 mL) was added dropwise at 0° C. After complete addition, the resulting mixture was allowed to stir overnight at rt. The reaction progress was monitored by RP-HPLC. The reaction mixture was poured into a vigorous stirred 1 M potassium sodium tartrate solution (120 mL) and stirred for another 30 min at rt. The reaction mixture was extracted with ethyl acetate (3×30 mL). The combined organic layers were washed with water and brine (each 30 mL). The organic layer was dried over MgSO₄. The solvent was removed under reduced pressure to obtain crude product. Purification by ISCO SiO₂ flash chromatography (0-100% ethyl acetate in hexane) yielded (4-amino-2-((prop-2-yn-1-yloxy)methyl)phenyl) methanol (0.65 g, 71%). ¹H NMR (500 MHZ, DMSO) δ 6.99 (d, J=8.1 Hz, 1H), 6.58 (d, J=2.4 Hz, 1H), 6.45 (dd, J=8.0, 2.4 Hz, 1H), 4.93 (s, 2H), 4.62-4.53 (m, 1H), 4.47 (s, 2H), 4.35 (d, J=5.4 Hz, 2H), 4.15 (d, J=2.4 Hz, 2H), 3.42 (t, J=2.4 Hz, 1H). HPLC (method A): t_{R}=4.8 min. MS (ESI): found m/z=174.2 [M−H₂O+H]⁺, calcd. m/z=174.1

### Step 5a: Synthesis Fmoc-Val-Ala-OH

The synthesis of Fmoc-Val-Ala-OH was performed as disclosed in WO 2017/149077.

### Step 5b: Synthesis Boc-Val-Ala-OH

The title compound is commercially available. CAS: 70396-18-8

### Example 1.2—Syntheses of Linker-Payload Intermediates

### Step 6a: Synthesis of Intermediate 1.1

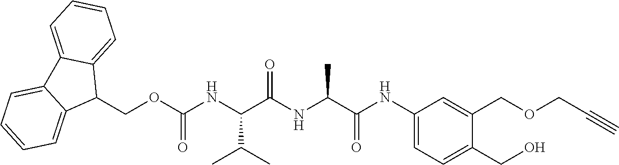 [SMILES: C#CCOCc1cc(NC(=O)[C@H](C)NC(=O)[C@@H](NC(=O)OCC2c3ccccc3-c3ccccc32)C(C)C)ccc1CO]

To a stirred solution of Fmoc-Val-Ala-OH (1.43 g, 3.41 mmol, 1 equiv.) and (4-amino-2-((prop-2-yn-1-yloxy)methyl)phenyl) methanol (0.65 g, 3.41 mmol, 1 equiv.) in dry THF (20 mL) was added N-Ethoxycarbonyl-2-ethoxy-1,2-dihydroquinoline (EEDQ, 1.05 g, 4.1 mmol, 1.2 equiv.). at room temperature. The resulting mixture was stirred at room temperature in the absence of light for 5 d. The solvent was removed under reduced pressure. The resulting crude intermediate was purified by flash chromatography on silica (gradient from 0-5% MeOH in DCM) to yield intermediate 1.1 as colorless solid (1.25 g, 63%). HPLC (method A): t_{R}=12.9 min. MS (ESI): found m/z=601.2 [M+NH₄]⁺, calcd. m/z=601.3

### Step 7: Synthesis of Intermediate 1.2

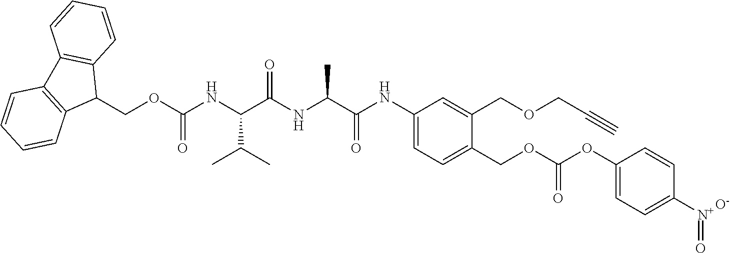

To a stirred solution of intermediate 1.1 (0.72 g, 1.24 mmol) in dry DMF (15 mL) were added bis-4-nitrophenyl-carbonate (0.75 g, 2.48 mmol, 2 equiv.) and N-ethyldiisopropylamine (DIPEA, 3.1 mmol, 0.53 ml, 2.5 equiv.) under an argon atmosphere. The reaction was allowed to stir at room temperature for 4 h. After completion, the solvent was removed under reduced pressure. The crude intermediate was purified by flash chromatography on silica (gradient from 0-100% ethylacetate in n-hexane) to yield intermediate 1.2 (0.75 g, 81%) as pale yellow solid after lyophilization from 1,4-dioxane:water (4:1). HPLC (method C): t_{R}=13.4 min. MS (ESI): found m/z=771.2 [M+Na]⁺, calcd. m/z=771.3

### Step 8a: Synthesis of Intermediate 1.3

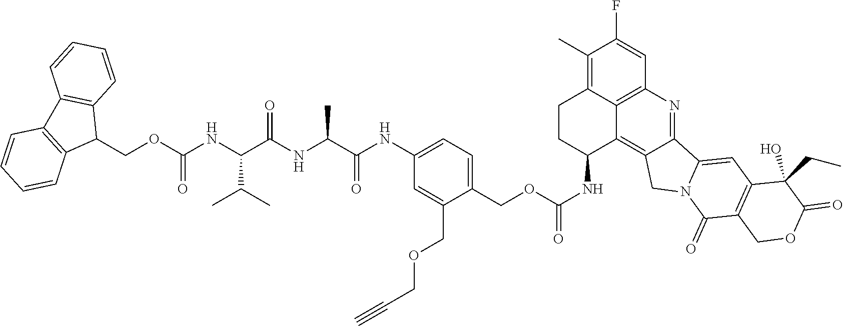 [SMILES: C#CCOCc1cc(NC(=O)[C@H](C)NC(=O)[C@@H](NC(=O)OCC2c3ccccc3-c3ccccc32)C(C)C)ccc1COC(=O)N[C@H]1CCc2c(C)c(F)cc3nc4c(c1c23)Cn1c-4cc2c(c1=O)COC(=O)[C@]2(O)CC]

To a stirring suspension of exatecan mesylate (0.58 g, 1.1 mmol, 1 equiv.) in dry DMF (20 mL) was added a freshly prepared solution of intermediate 1.2 (0.82 g; 1.1 mmol, 1 equiv.) and 1-hydroxybenzotriazole (HOBt, 0.042 g, 0.275 mmol, 0.25 equiv.) in dry DMF (20 mL). DIPEA was added to the resulting mixture (0.28 g, 2.16 mmol, 0.37 mL, 2 equiv.) and 2,6-lutidine (141 mmol, 16.3 mL, 129 equiv.). The reaction mixture was stirred at room temperature overnight and the progress monitored by RP-HPLC. The reaction mixture was concentrated under reduced pressure. Purification by flash chromatography on silica (gradient from 0-5% MeOH in DCM) and lyophilization from 1,4-dioxane:water (4:1) yielded intermediate 1.3 (1.08 g, 95%) as gray solid. HPLC (method D): t_{R}=13.0 min. MS (ESI): found m/z=1045.3 [M+H]⁺, calcd. m/z=1045.4

### Step 6b: Synthesis of Intermediate 1.4

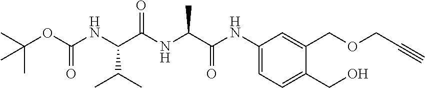 [SMILES: C#CCOCc1cc(NC(=O)[C@H](C)NC(=O)[C@@H](NC(=O)OC(C)(C)C)C(C)C)ccc1CO]

To a stirred solution of Boc-Val-Ala-OH (0.57 g, 1.98 mmol, 1 equiv.) and (4-amino-2-((prop-2-yn-1-yloxy)methyl)phenyl) methanol (0.36 g, 1.89 mmol, 1.05 equiv.) in dry THF (9 mL) was added N-Ethoxycarbonyl-2-ethoxy-1,2-dihydroquinoline (EEDQ, 0.56 g, 2.27 mmol, 1.2 equiv.). at room temperature. The resulting mixture was stirred at room temperature in the absence of light for 5 h. The solvent was removed under reduced pressure. The resulting crude intermediate was purified by ISCO SiO₂ chromatography (0-50% hexane/ethyl acetate) to yield intermediate 1.4 as colorless solid (0.81 g, 93%). ¹H NMR (500 MHZ, DMSO) δ 9.89 (s, 1H), 7.98 (q, J=7.1 Hz, 1H), 7.54 (d, J=7.5 Hz, 2H), 7.32 (d, J=8.5 Hz, 1H), 4.95 (t, J=5.4 Hz, 1H), 4.55 (s, 2H), 4.48 (d, J=5.2 Hz, 2H), 4.43 (t, J=7.0 Hz, 1H), 4.19 (d, J=2.4 Hz, 2H), 3.83 (s, 1H), 3.44 (t, J=2.4 Hz, 1H), 1.97 (s, 1H), 1.39 (s, 9H), 1.30 (d, J=7.0 Hz, 3H), 0.85 (dd, J=23.2, 6.8 Hz, 6H). HPLC (method A): t_{R}=10.9 min. MS (ESI): found m/z=461.9 [M+H]⁺, calcd. m/z=462.26

### Step 7b: Synthesis of Intermediate 1.5

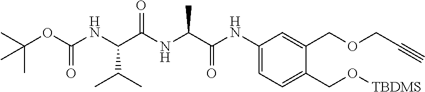

To a stirred solution of intermediate 1.4 (0.3 g, 0.65 mmol, 1 equiv.) and imidazole (0.07 g, 0.96 mmol, 1.5 equiv.) in dry DMF (2 mL) was added a solution of tert-butyldimethylsilyl chloride (0.12 g, 0.81 mmol, 1.25 equiv.) in dry DMF (1 mL) dropwise. The reaction was stirred at room temperature and monitored by TLC and RP-HPLC. After 2 h the solvent was removed under reduced pressure. The residue was taken up in 100 mL ethyl acetate and washed with 0.2 N citric acid, sat. sodium bicarbonate, and brine (each 100 mL). The organic layer was dried over MgSO₄. After filtration the solvent was removed under reduced pressure. The resulting crude intermediate was purified by ISCO SiO₂ chromatography (0-50% hexane/ethyl acetate) to yield intermediate 1.5 as colorless solid (0.22 g, 58%). HPLC (method P): t_{R}=16.7 min. MS (ESI): found m/z=576.0 [M+H]⁺, calcd. m/z=576.35

### Step 8b: Synthesis of Intermediate 1.6

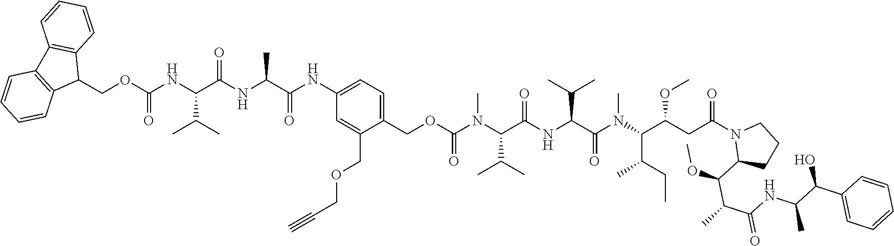 [SMILES: C#CCOCc1cc(NC(=O)[C@H](C)NC(=O)[C@@H](NC(=O)OCC2c3ccccc3-c3ccccc32)C(C)C)ccc1COC(=O)N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Intermediate 1.6 was synthesized as described for intermediate 1.3 but using MMAE and the reaction mixture was stirred for 45 h. Intermediate 1.6 (175.37 mg, 56%) was obtained as colorless solid. UHPLC (method R with TFA as additive at 290 nm): t_{R}=4.9 min. MS (ESI): found m/z=1327.8 [M+H]⁺, calcd. m/z=1327.75

### Step 8c: Synthesis of Intermediate 1.7

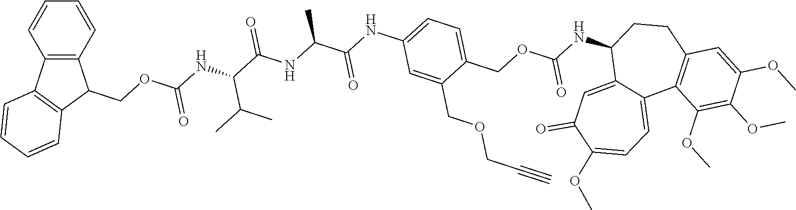 [SMILES: C#CCOCc1cc(NC(=O)[C@H](C)NC(=O)[C@@H](NC(=O)OCC2c3ccccc3-c3ccccc32)C(C)C)ccc1COC(=O)N[C@H]1CCc2cc(OC)c(OC)c(OC)c2-c2ccc(OC)c(=O)cc21]

Intermediate 1.7 was synthesized as described for intermediate 1.3. However, RP-HPLC analysis of the reaction mixture indicated that the reaction went to completion after 3.5 h stirring. Intermediate 1.7 (182.24 mg, 67%) was obtained as yellowish solid. UHPLC (method R with TFA as additive at 290 nm): t_{R}=4.1 min. MS (ESI): found m/z=967.2 [M+H]⁺, calcd. m/z=967.41

### Step 8d: Synthesis of Intermediate 1.8

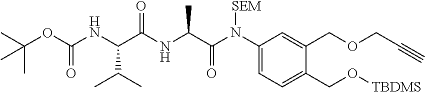

To a cooled solution (0° C.) of intermediate 1.5 (0.21 g, 0.38 mmol, 1 equiv.) in dry THF (7.6 mL) was added a solution of Lithium 1,1,1-trimethyl-N-(trimethylsilyl) silanaminide (LiHMDS, 1 M in THF, 0.57 mL, 0.57 mmol, 1.5 equiv.) dropwise. After complete addition, the resulting mixture was allowed to stir for 10 min. Afterwards 2-(trimethylsilyl)ethoxymethyl chloride (SEM-Cl, 0.13 mL, 0.76 mmol, 2 equiv.) was added and the reaction mixture was allowed to stir for another 15 min at 0° C. The ice bath was removed, and the reaction mixture was stirred for additional 30 min from 0° C. to room temperature. Upon completion of the reaction, saturated sodium citrate solution (2.5 mL, pH=6.4) was added. For phase separation the resulting mixture was diluted with 20 mL ethyl acetate. The organic layer was washed with sodium citrate buffer (pH=4.76), sat. sodium bicarbonate, and brine (each 2.5 mL). The organic layer was dried over MgSO₄. After filtration the solvent was removed under reduced pressure. The resulting crude intermediate was purified by ISCO SiO₂ chromatography (0-50% hexane/tert-butyl methyl ether) to yield intermediate 1.8 as colorless solid (0.25 g, 94%). HPLC (method P): t_{R}=16.1 min. MS (ESI): found m/z=706.1 [M+H]⁺, calcd. m/z=706.43

### Step 8c: Synthesis of Intermediate 1.9

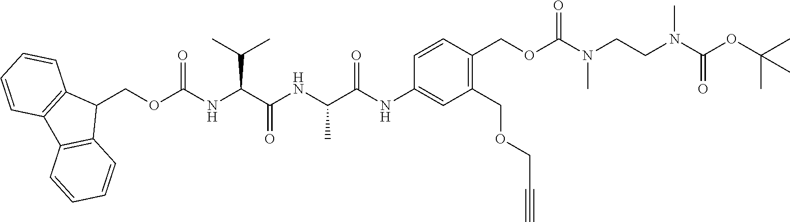 [SMILES: C#CCOCc1cc(NC(=O)[C@H](C)NC(=O)[C@@H](NC(=O)OCC2c3ccccc3-c3ccccc32)C(C)C)ccc1COC(=O)N(C)CCN(C)C(=O)OC(C)(C)C]

To a stirred solution of intermediate 1.2 (1 g, 1.34 mmol, 1 equiv.) in dry DMF (35 mL) was added tert-butyl methyl (2-(methylamino)ethyl)carbamate (0.3 g, 1.6 mmol, 1.2 equiv.). at room temperature. The resulting mixture was stirred at room temperature under argon atmosphere for 1 h. The reaction mixture was concentrated under reduced pressure. Then, diluted with 75 mL ethyl acetate/THF 5:1 and washed with 10% citric acid, 5% sodium bicarbonate, and brine (each 50 mL). The organic layer was dried over MgSO₄. After filtration the solvent was removed under reduced pressure. The resulting crude intermediate was purified by ISCO SiO₂ chromatography (0-100% hexane/ethyl acetate). After evaporation of the volatiles, the residue was taken up in 1,4-dioxane and lyophilized to yield intermediate 1.9 as colorless solid (0.95 g, 89%). HPLC (method A): t_{R}=15.2 min. MS (ESI): found m/z=798.2 [M+H]⁺, calcd. m/z=798.41

### Step 9a: Synthesis of Intermediate 1.10

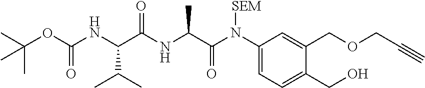 [SMILES: C#CCOCc1cc(N(COCC[Si](C)(C)C)C(=O)[C@H](C)NC(=O)[C@@H](NC(=O)OC(C)(C)C)C(C)C)ccc1CO]

To a stirred solution of intermediate 1.8 (0.25 g, 0.36 mmol, 1 equiv.) in dry THF (3.6 mL) was added a solution of N,N,N-tributylbutan-1-aminium fluoride (TBAF, 1 M in THF, 0.43 mL, 0.43 mmol, 1.2 equiv.). Upon completion (˜15 min), the reaction mixture was subjected to ISCO SiO₂ chromatography (0-100% hexane/tert-butyl methyl ether) to yield intermediate 1.10 as colorless solid (0.23 g, quant.). HPLC (method A): t_{R}=14.6 min. MS (ESI): found m/z=591.9 [M+H]⁺, calcd. m/z=592.34

### Step 9b: Synthesis of Intermediate 1.11

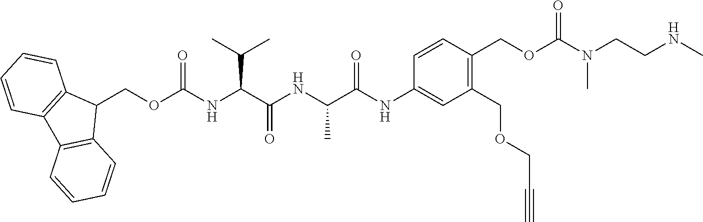 [SMILES: C#CCOCc1cc(NC(=O)[C@H](C)NC(=O)[C@@H](NC(=O)OCC2c3ccccc3-c3ccccc32)C(C)C)ccc1COC(=O)N(C)CCNC]

To a stirred solution of intermediate 1.9 (0.27 g, 0.33 mmol, 1 equiv.) in dry methylene chloride (3 mL) was added TFA (0.43 mL, 6.61 mmol, 19.75 equiv.). at room temperature. The resulting mixture was stirred at room temperature. Upon completion (˜2 h), the reaction mixture was added to 13 mL ice cold MTBE. The resulting precipitate was isolated by centrifugation, resuspended in 10 mL cold MTBE and centrifuged again. The crude product (0.24 g) was taken up in 5 mL DMF and purified by prep. RP-HPLC to yield 190.4 mg (70%) of intermediate 1.11 (TFA-Salt) as colorless solid. HPLC (method A): t_{R}=15.2 min. MS (ESI): found m/z=798.2 [M+H]⁺, calcd. m/z=798.41

### Step 10a: Synthesis of Intermediate 1.12

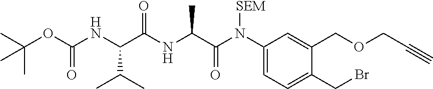 [SMILES: C#CCOCc1cc(N(COCC[Si](C)(C)C)C(=O)[C@H](C)NC(=O)[C@@H](NC(=O)OC(C)(C)C)C(C)C)ccc1CBr]

To a cooled solution (5° C.) of intermediate 1.10 (0.13 g, 0.22 mmol, 1 equiv.) in dry methylene chloride (1.3 mL) in inert atmosphere was added methanesulfonic anhydride (dissolved in 0.7 mL dry DCM, 0.046 g, 0.26 mmol, 1.2 equiv.) and DIPEA (0.09 mL, 0.53 mmol, 2.4 equiv.). The reaction was monitored by TLC and RP-HPLC. After 3 h the sulfonation was complete (if necessary, additional methanesulfonic anhydride can be added). A solution of LiBr (0.095 g, 1.11 mmol, 5 eq.) in 1.7 mL dry THF was added, and reaction mixture was allowed to stir for another 10 min at 5° C. The cooling bath was removed, and reaction was stirred until completion at room temperature. Upon completion (˜2 h), 0.2 M sodium citrate solution (5 mL, pH=6.4) was added. For phase separation the resulting mixture was diluted with 50 mL MTBE. The organic layer was washed with 0.2 M sodium citrate buffer (5 mL, pH=4.76), sat. sodium bicarbonate, and brine (each 50 mL). The organic layer was dried over MgSO₄. After filtration the solvent was removed under reduced pressure. The resulting crude intermediate was purified by flash chromatography (SiO₂, 0-100% hexane/tert-butyl methyl ether). The solvent was removed, and the residue was taken up in 1,4-dioxane and lyophilized to yield intermediate 1.12 as colorless lyophilizate (0.052 g, 36%). HPLC (method P): t_{R}=9.3 min. MS (ESI): found m/z=653.8 [M+H]⁺, calcd. m/z=654.26

### Step 10b: Synthesis of Intermediate 1.13

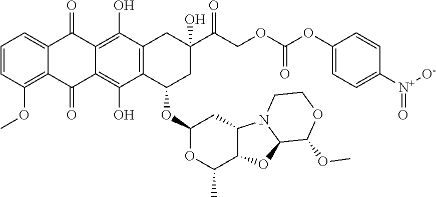 [SMILES: COc1cccc2c1C(=O)c1c(O)c3c(c(O)c1C2=O)C[C@@](O)(C(=O)COC(=O)Oc1ccc([N+](=O)[O-])cc1)C[C@@H]3O[C@H]1C[C@H]2[C@H](O[C@@H]3[C@@H](OC)OCCN32)[C@H](C)O1]

The intermediate was prepared as described in Holte, et al., Bioorg. & Med. Chem., Vol. 30, 24, 2020, 127640. The target product was obtained in 17% yield as red solid. MS (ESI): found m/z=806.8 [M+H]⁺, calcd. m/z=807.22 [M+H]⁺.

### Step 10c: Synthesis of Intermediate 1.14 (Deacetylcolchicine

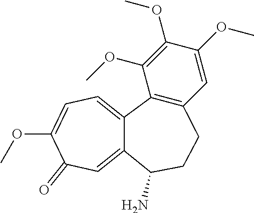

The intermediate was prepared as described in WO 2016/019340. The target product was obtained in 99% yield (over 3 steps) as yellowish solid. UHPLC (method V): t_{R}=1.0 min. ¹H NMR (500 MHZ, DMSO) 8.46 (s, 3H), 7.20-7.13 (m, 2H), 7.10 (d, J=11.2 Hz, 1H), 6.81 (s, 1H), 3.91 (s, 3H), 3.85 (s, 3H), 3.83-3.74 (m, 1H), 3.78 (s, 3H), 3.61 (s, 3H), 2.66 (d, J=6.8 Hz, 1H), 2.34-2.26 (m, 2H), 1.93 (dd, J=11.8, 5.4 Hz, 1H). MS (APCI): found m/z=357.8 [M+H]⁺, calcd. m/z=358.16

### Step 11a: Synthesis of Intermediate 1.15

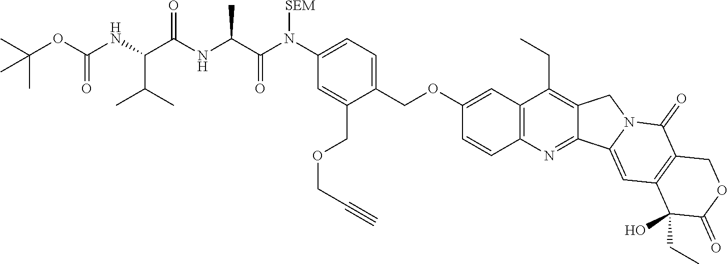 [SMILES: C#CCOCc1cc(N(COCC[Si](C)(C)C)C(=O)[C@H](C)NC(=O)[C@@H](NC(=O)OC(C)(C)C)C(C)C)ccc1COc1ccc2nc3c(c(CC)c2c1)Cn1c-3cc2c(c1=O)COC(=O)[C@]2(O)CC]

SN-38 (0.036 g, 0.092 mmol, 1 equiv.) was suspended in 1.6 mL dry DMF. To 0.8 mL dry DMF was added potassium tert-butoxide (KOtBu, 1 M in THF, 0.14 mL, 0.14 mmol, 1.5 equiv.). The resulting KOtBu-solution was added dropwise at room temperature to the reaction flask un an argon atmosphere. After 5 min intermediate 1.12 (0.06 g, 0.092 mmol, 1 equiv.) was dissolved in 0.6 mL dry DMF and added dropwise to reaction mixture. The solvent was removed under reduced pressure. The resulting crude intermediate was purified by flash chromatography (SiO₂, 0-50% hexane/ethyl acetate) to yield intermediate 1.9 as colorless solid (0.04 g, 52%). HPLC (method A (380 m)): t_{R}=15.9 min. MS (ESI): found m/z=966.2 [M+H]⁺, calcd. m/z=966.47

### Step 11b: Synthesis of Intermediate 1.16

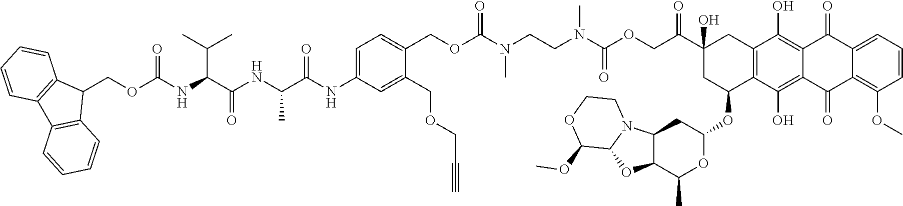 [SMILES: C#CCOCc1cc(NC(=O)[C@H](C)NC(=O)[C@@H](NC(=O)OCC2c3ccccc3-c3ccccc32)C(C)C)ccc1COC(=O)N(C)CCN(C)C(=O)OCC(=O)[C@]1(O)Cc2c(O)c3c(c(O)c2[C@@H](O[C@H]2C[C@H]4[C@H](O[C@@H]5[C@@H](OC)OCCN54)[C@H](C)O2)C1)C(=O)c1c(OC)cccc1C3=O]

To stirred solution of intermediate 1.13 (31.62 mg, 0.039 mmol, 1 equiv.) in 0.54 mL dry DMF was added 0.54 mL dry DMF containing intermediate 1.11 (47.55 mg, 0.059 mmol, 1.5 equiv.) and DIPEA (13.62 μL, 0.078 mmol, 2 equiv.) under an argon atmosphere. The resulting mixture was stirred at room temperature for 1.5 h. Upon completion, the reaction mixture was directly purified without any pretreatment by prep. HPLC to yield 26.9 mg (50%) of intermediate 1.16 as red solid. HPLC (method A without additive): t_{R}=15.8 min. MS (ESI): found m/z=1365.4 [M+H]⁺, calcd. m/z=1365.55

### Synthesis of SN-38-PNP

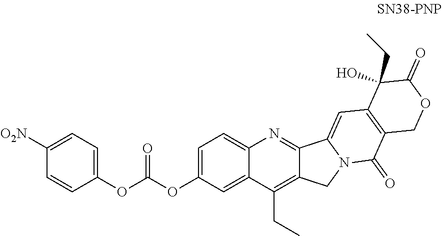 [SMILES: CCc1c2c(nc3ccc(OC(=O)Oc4ccc([N+](=O)[O-])cc4)cc13)-c1cc3c(c(=O)n1C2)COC(=O)[C@]3(O)CC]

To a suspension SN-38 (200 mg, 0.51 mmol, 1 equiv.) and 4-nitrophenyl chloroformiate (123.3 mg, 0.61 mmol, 1.2 equiv.) in THF (12 mL) was added DIPEA (129.6 μL, 0.74 mmol, 1.5 equiv.) at 0° C. under argon. The resulting solution was warmed to room temperature and was stirred for additional 1.5 h. The solution was concentrated in vacuo and the resulting residue purified by prep HPLC affording SN-38-PNP (169.4 mg, 60%) as yellowish solid. C₂₉H₂₃N₃O₉. HPLC (method A): t_{R}=10.0 min. MS (ESI): found m/z=558.0, calcd. m/z=558.1 [M+H]⁺.

### Synthesis of Intermediate 1.17

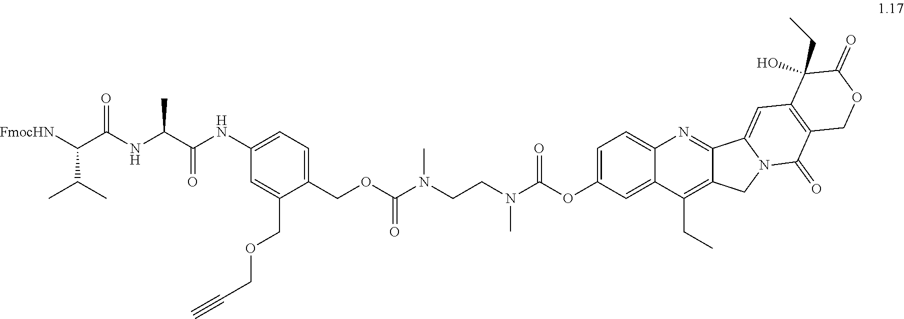

To a solution of intermediate 1.11 (69.6 mg, 85.7 μmol, 1 equiv.) and SN-38-PNP (47.8 mg, 85.7 μmol, 1 equiv.) in DMF (2 mL) was added DIPEA (29.9 μL, 171.4 μmol, 2 equiv.) under argon. The resulting solution was stirred at room temperature for 2.25 h and purified by prep HPLC affording intermediate 1.17 (78 mg, 82%) as yellowish solid. C₆₂H₆₅N₇O₁₃. HPLC (method A): t_{R}=14.0 min. MS (ESI): found m/z=1168.8, calcd. m/z=1116.5 [M+H]⁺.

### NAMPTi

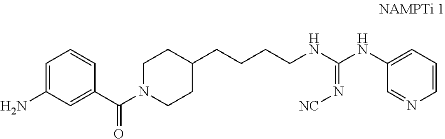 [SMILES: N#C/N=C(\NCCCCC1CCN(C(=O)c2cccc(N)c2)CC1)Nc1cccnc1]

NAMPTi 1 was synthesized as described in WO2018201087.

### Synthesis of Intermediate 1.18

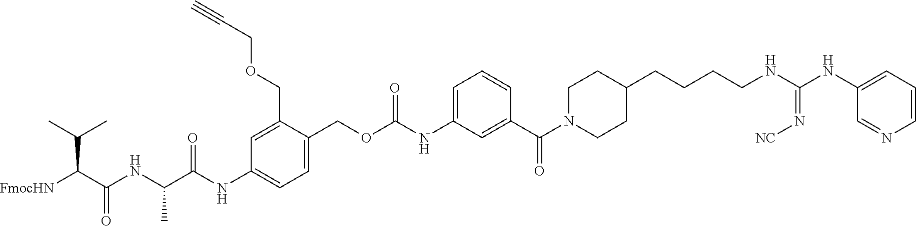

Intermediate 1.18 was synthesized as described for intermediate 1.3 but using NAMPTi 1 (150 mg, 0.36 mmol, 1 equiv.), intermediate 1.2 (402 mg; 0.54 mmol, 1.5 equiv.), 1-hydroxy-7-azabenzotriazole (HOAt, 600 μL of a 0.6 M solution in DMF, 0.36 mmol, 1 equiv.) and 2,6-lutidine (5.5 mL, 45 mmol, 125 equiv.) in dry DMF (9.52 mL). The pH of the reaction mixture was regularly adjusted with DIPEA maintaining a pH of 8. The reaction mixture was stirred at room temperature for 6 d and purification by preparative HPLC afforded intermediate 1.18 (120.6 mg, 32%) as colorless solid. C₅₈H₆₄N₁₀O₈. HPLC (method AA): t_{R}=12.8 min. MS (ESI): found m/z=1029.7 [M+H]⁺, calcd. m/z=1029.5.

### TLR7 Agonists

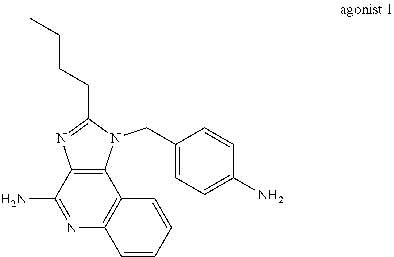 [SMILES: CCCCc1nc2c(N)nc3ccccc3c2n1Cc1ccc(N)cc1]

Agonist 1 was synthesized as described in WO2022187809.

### Synthesis of Intermediate 1.19

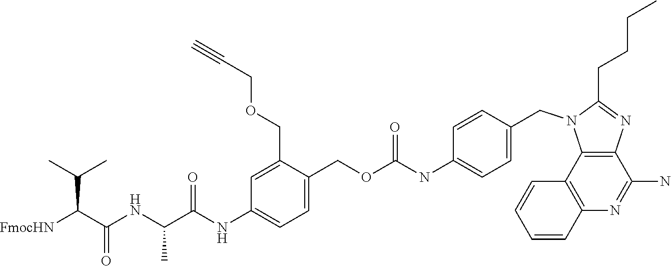

Intermediate 1.19 was synthesized as described for intermediate 1.3 but using agonist 1 and was stirred for 5 d. Intermediate I.19 (184.1 mg, 33%) was obtained as yellowish solid. C₅₆H₅₈N₈O₇. HPLC (method A): t_{R}=12.0 min. MS (ESI): found m/z=955.5 [M+4H]⁴⁺, calcd. m/z=955.4 [M+4H]⁴⁺.

### Synthesis of 1.20

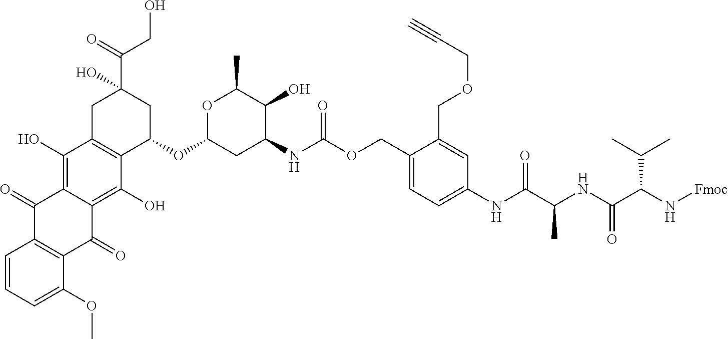

Intermediate 1.20 was synthesized as described for intermediate 1.3 from intermediate 1.2 (258 mg, 0.344 mmol, 1 equiv.) and doxorubicin hydrochloride (200 mg, 1.0 eq.). Intermediate 1.20 (395 mg, 97%) was obtained as red solid. HPLC method A (480 nm): t_{R}=14.0 min. MS (ESI): found m/z=1173.3 [M+Na]⁺, calcd. m/z=1175.41

### Synthesis of 1.21

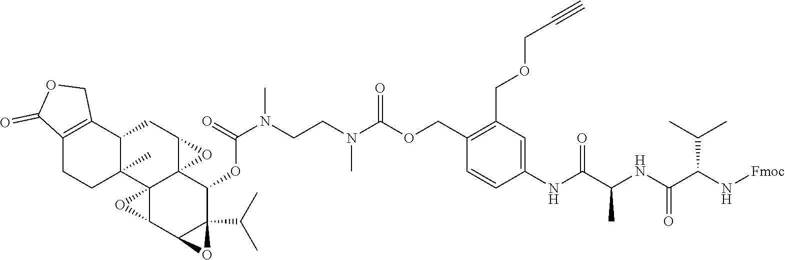

To the 4-nitrophenyl-carbonate derivative of tripolide, described in WO2022040487 (44.60 mg, 84.87 μmol, 1 equiv.) and intermediate 1.11 (103.35 mg, 127.30 μmol, 1.5 equiv.) in 1 mL dry DMF was added DIPEA (28.9 μL, 169.74 μmol, 2 equiv.) and the mixture was stirred under argon atmosphere at room temperature. After complete conversion (1 h 45 min) the solution was acidified with TFA (13 μL. 2 equiv.) filtrated (0.2 μm Nylon) and purified by prep. HPLC to result Intermediate 1.21 (72.95 mg, 79%) as colorless solid. HPLC method A (260 nm): t_{R}=13.4 min. MS (ESI): found m/z=1084.8 [M+Na]⁺, calcd. m/z=1084.5

### Intermediate 1.22

### Step 1

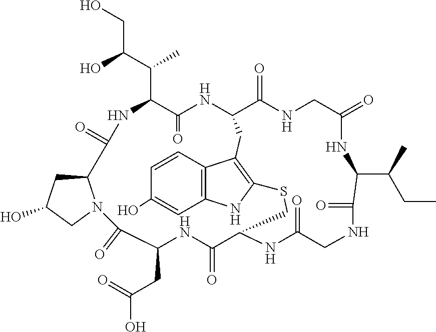 [SMILES: CC[C@H](C)[C@@H]1NC(=O)CNC(=O)[C@@H]2Cc3c([nH]c4cc(O)ccc34)SC[C@H](NC(=O)CNC1=O)C(=O)N[C@@H](CC(=O)O)C(=O)N1C[C@H](O)C[C@H]1C(=O)N[C@@H]([C@@H](C)[C@@H](O)CO)C(=O)N2]

S-deoxy-b-amanitin [CAS: 2417149-39-2] was synthesized according to Lutz et al. Angewandte Chemie, International Edition (2020), 59 (28), 11390-11393.

### Step 2

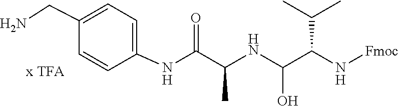

Fmoc-Val-Ala-(4-amionomethyl)-anilide Linker [CAS: 2803767-21-5] was synthesized according to WO 2022/155347

### Step 3

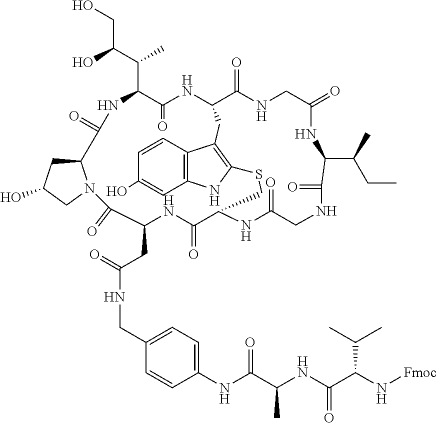

To the product of step 1, S-deoxy-b-amanitin (94 mg, 104 μmol) dissolved in 3 mL dry DMF was added 1-hydroxybenzotriazole hydrate (159 mg, 1040 μmol, 10 equiv.) and N,N′-diisopropylcarbodiimide (162 μL, 1040 μmol, 10 equiv.) and the mixture stirred for 1 h followed by addition of step 2 product (98 g, 156 μmol, 1.5 equiv.) and DIPEA (53 μL, 312 μmol, 3 equiv.) and stirring was continued overnight. The reaction mixture was subsequently added dropwise to 40 mL ice-cooled MTBE and the resulting precipitate was isolated by centrifugation, washed with 40 mL fresh MTBE and dried in vaccuo.

The crude product was further purified on prep. HPLC to result in 115 mg (79%) step 3 product.

### Step 4

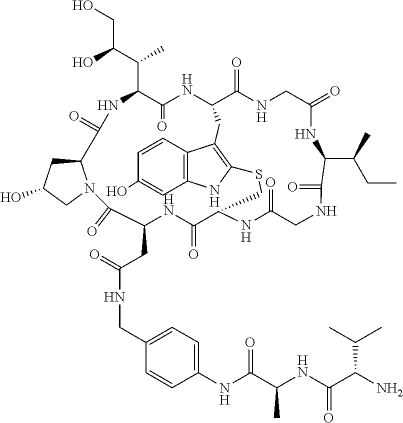 [SMILES: CC[C@H](C)[C@@H]1NC(=O)CNC(=O)[C@@H]2Cc3c([nH]c4cc(O)ccc34)SC[C@H](NC(=O)CNC1=O)C(=O)N[C@@H](CC(=O)NCc1ccc(NC(=O)[C@H](C)NC(=O)[C@@H](N)C(C)C)cc1)C(=O)N1C[C@H](O)C[C@H]1C(=O)N[C@@H]([C@@H](C)[C@@H](O)CO)C(=O)N2]

Step 3 product (67.02 mg, 47.85 μmol) was dissolved in 3 mL DMF and 100 μL (960 μmol) diethylamine was added and the reaction stirred for 1 h at room temperature. The volatiles were evaporated in vaccuo and the remaining were co-evaporated with 3 mL DMF. The crude was then dissolved in 1 mL of methanol and added dropwise to 40 mL ice-cooled MTBE and the resulting precipitate was isolated by centrifugation, washed with 40 mL fresh MTBE and dried in vaccuo. The intermediate 1.22 (69.5 mg) was used without further purification.

### Example 1.3—Synthesis of Intermediate 2.1 & 2.2

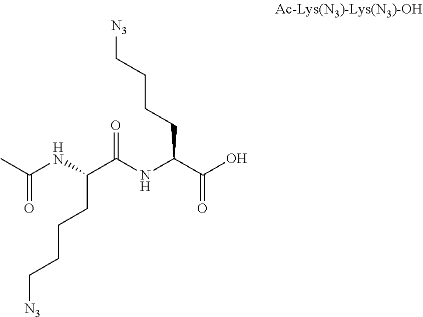 [SMILES: CC(=O)N[C@@H](CCCCN=[N+]=[N-])C(=O)N[C@@H](CCCCN=[N+]=[N-])C(=O)O]

2-chlorotrityl resin (4 g) were swollen for 10 min in abs. DCM (20 mL). The suspension was filtered, a solution of Fmoc-Lys(N₃)—OH (1.578 g, 4 mmol, 1 equiv.), DIPEA (3.48 mL, 20 mmol, 5 equiv.) in abs. DCM (20 mL) added and shaken for 2 h at room temperature. The suspension was filtered, the resin washed with DMF (3×1 min) and the capping solution (20 mL, MeOH/DIPEA/DCM 1/1/8) was added and shaken for 10 min at room temperature. The suspension was filtered and the capping procedure repeated once. The resin was washed with DMF (3×1 min) and DCM (3×1 min) and the resin dried in vacuo. The loading of the resin was determined as described in J. Pept. Sci. 2017, 23, 757-762.

For the next step, the resin was swollen in DMF for 10 min. The suspension was filtered and a Fmoc-deprotection solution of 20% piperidine in DMF added to the resin. The suspension was incubated for 20 min, filtered and the deprotection procedure repeated once. Then, the resin was washed with DMF (3×1 min) and DCM (3×1 min). The resin was swollen in DMF for 10 min, filtered and a solution of Fmoc-Lys(N₃)—OH (3 equiv.), TBTU (3 equiv.) and DIPEA (6 equiv.) in DMF added. The suspension was shaken for 2 h at room temperature. The resin was washed with DMF (3×1 min) and DCM (3×1 min) and the Fmoc-group removed with 20% piperidine as described above. For acetylation, the resin was swollen in DMF for 10 min, the suspension filtered and a solution of acetic anhydride (3 equiv.) and pyridine (3 equiv.) in DMF added. The suspension was incubated for 2 h at room temperature, filtered, the resin washed with DMF (3×1 min) and DCM (3×1 min) and dried in vacuo.

For cleavage, the resin was treated with a solution of hexafluoroisopropanol in DCM (1:4, 20 mL) for 15 min. The solution is collected and the resin treated again with hexafluoroisopropanol in DCM (1:4, 20 mL). The resin is washed with hexafluoroisopropanol in DCM (1:4) and the combined filtrates concentrated in vacuo. The resulting crude peptide was purified by RP-HPLC and Ac-Lys(N₃)-Lys(N₃)—OH (731 mg, 50%) obtained as colorless lyophilizate.

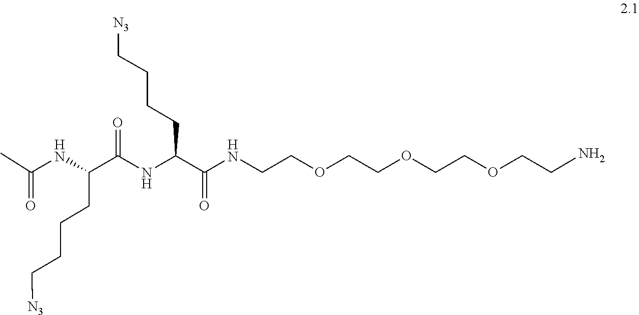 [SMILES: CC(=O)N[C@@H](CCCCN=[N+]=[N-])C(=O)N[C@@H](CCCCN=[N+]=[N-])C(=O)NCCOCCOCCOCCN]

Ac-Lys(N₃)-Lys(N₃)—OH (731 mg, 1.98 mmol, 1 equiv.) and HOBt (910 mg, 5.94 mmol, 3 equiv.) were dissolved in DMF (8 mL). DIC (920 μL, 5.9 mmol, 3 equiv.) was added, the solution stirred for 5 min at room temperature after which a solution of Boc-Amino-PEG (3)-Amine (CAS-Nr. 101187-40-0, 868 mg, 2.97 mmol, 1.5 equiv.) in DMF (4 mL) was added followed by DIPEA (1.04 mL, 5.98 mmol, 3 equiv.). The reaction was stirred at room temperature and followed by RP-HPLC. After 20 h, the solution was concentrated and the residue dissolved in ethyl acetate. The organic layer was washed with 0.2 M citric acid (1×50 mL), sodium bicarbonate solution (3×50 mL) and brine. The organic layer was dried over magnesium sulfate and concentrated in vacuo giving 1.428 g of crude product. Purification by RP-HPLC followed by freeze drying in the presence of HCl (1 equiv.) for two times afforded intermediate 2.1 (705 mg, 66%) as HCl-salt. HPLC (method H): t_{R}=11.2 min. MS (ESI): found m/z=543.3 [M+H]⁺, calcd. m/z=543.3 [M+H]⁺.

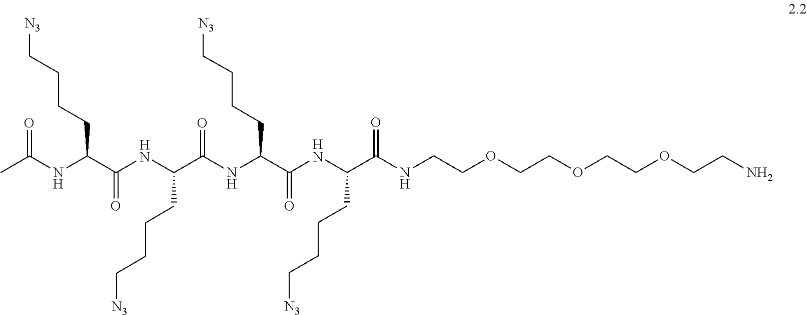 [SMILES: CC(=O)N[C@@H](CCCCN=[N+]=[N-])C(=O)N[C@@H](CCCCN=[N+]=[N-])C(=O)N[C@@H](CCCCN=[N+]=[N-])C(=O)N[C@@H](CCCCN=[N+]=[N-])C(=O)NCCOCCOCCOCCN]

Intermediate 2.2 was synthesized as described for intermediate 2.1 but with additional two more cycles of Lys(N₃)-coupling obtaining a tetrapeptide. Purification by RP-HPLC followed by freeze drying afforded intermediate 2.2 (139 mg, 37%) as sticky colorless solid. HPLC (method H): t_{R}=9.3 min. MS (ESI): found m/z=851.6 [M+H]⁺, calcd. m/z=851.51 [M+H]⁺.

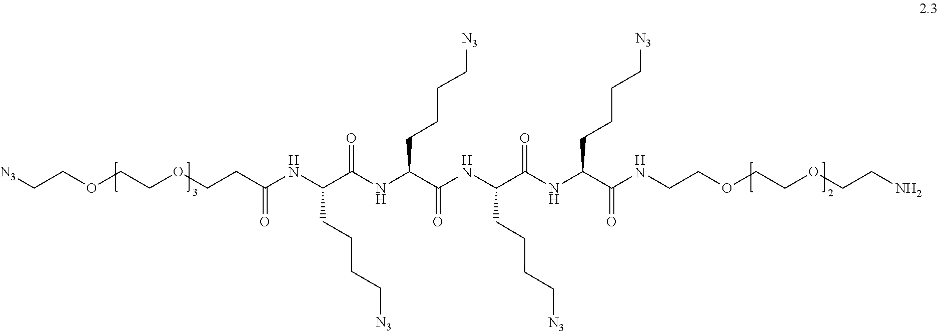

Intermediate 2.3 was synthesized as described for intermediate 2.1 but with additional two more cycles of Lys(N₃)-coupling obtaining a tetrapeptide and replacing acetic anhydride with azido-dPEG4-NHS ester [944251-24-5] for N-terminal capping. Purification by RP-HPLC followed by freeze drying afforded intermediate 2.3 as sticky colorless solid. HPLC (method A): t_{R}=10.1 min. MS (ESI): found m/z=1082.67 [M+H]⁺, calcd. m/z=1082.63 [C43H80N21O12]⁺.

Intermediate 2.4 was synthesized in two steps:

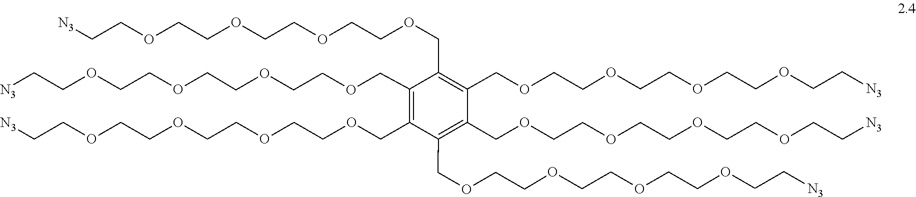 [SMILES: [N-]=[N+]=NCCOCCOCCOCCOCc1c(COCCOCCOCCOCCN=[N+]=[N-])c(COCCOCCOCCOCCN=[N+]=[N-])c(COCCOCCOCCOCCN=[N+]=[N-])c(COCCOCCOCCOCCN=[N+]=[N-])c1COCCOCCOCCOCCN=[N+]=[N-]]

Step 1: Under argon atmosphere 128 mg (4.06 mmol) sodium hydride (60% suspension in mineral oil) was washed two times with 5 mL dry THF and suspended again in 5 mL THF. A solution of 653 mg (2901 μmol=8 eq.) azido-PEG₄-alcohol [CAS 86770-67-4, TCI, A2294] in 5 mL THF was added dropwise and stirred at room temperature until gas evolution stopped (15 min). Solid hexakis(bromomethyl)benzene, 231 mg (363 mmol, CAS: 3095-73-6, TCI H0901) added in portions and the mixture was stirred overnight. The reaction mixture was poured in 20 ml saturated ammonium chloride solution, diluted with 20 mL ethyl acetate. The organic phase was separated and washed with 20 mL brine, dried (MgSO₄) and evaporated to dryness. The crude was purified on silica gel with a gradient of 0 to 50% acetone in hexane resulting in 392 mg (79%) hexaazide as slightly yellowish oil. MS (ESI): found m/z=1482.67 [M+NH₄]⁺, calcd. m/z=1482.81. ¹H NMR (500 MHZ, D6-DMSO) δ 4.59 (s, 16H), 3.63 (dd, J=6.1, 3.7 Hz, 15H), 3.61-3.48 (m, 72H), 3.39-3.33 (m, 14H). 13C NMR (126 MHZ, D6-DMSO) δ 137.25, 69.75, 69.73, 69.70, 69.65, 69.62, 69.45, 69.12, 65.60, 49.93.

Step 2: Step 1 product, 139 mg (95 μmol) was dissolved in 4.5 mL THF and triphenylphosphine, 25 mg (95 μmol) was added. After dissolution of the phosphine 0.5 ml water was added and the mixture was stirred at room temperature overnight in an open vessel and evaporated to dryness subsequently. The crude was freed from triphenylphosphine oxide and a small amount of starting material on silica gel with a Gradient of 0 to 10% methanol in DCM+0.1% triethylamine. The product containing fractions were evaporated and purified on prep. HPLC to result in 29.6 mg (20%) intermediate 2.4. HPLC (method A) t_{R}=11.2 min. MS (ESI): found m/z=1439.75 [M+2H]²⁺, calcd. m/z=1439.80 [C₆₀H₁₁₁N₁₆O₂₄]²⁺.

### Intermediate 2.5

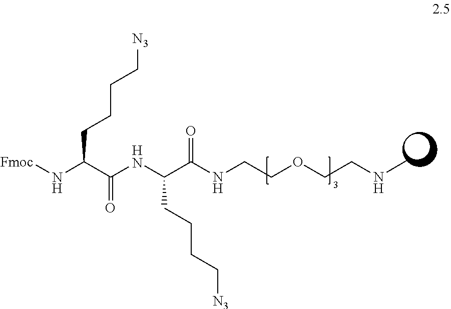

2-chlorotrityl resin (1 g) were swollen for 10 min in abs. DCM (5 mL). The suspension was filtered, a solution of 1,11-Diamino-3,6,9-trioxaundecane (531 mg, 2.76 mmol, 2 equiv.) in abs. DCM (5 mL) added and shaken for 2 h at room temperature. The suspension was filtered, the resin was washed with DCM (3×1 min) and the capping solution (5 mL, MeOH/DIPEA/DCM 1/1/8) was added and shaken for 10 min at room temperature. The suspension was filtered and the capping procedure repeated once. The resin was washed with DMF (3×1 min) and DCM (3×1 min) and the resin dried in vacuo.

For the next step, the resin was swollen in DMF for 10 min. The suspension was filtered and a Fmoc-deprotection solution of 20% piperidine in DMF added to the resin. The suspension was incubated for 20 min, filtered and the deprotection procedure repeated once. Then, the resin was washed with DMF (3×1 min) and DCM (3×1 min). The resin was swollen in DMF for 10 min, filtered and a solution of Fmoc-Lys(N₃)—OH (3 equiv.), TBTU (3 equiv.) and DIPEA (6 equiv.) in DMF added. The suspension was shaken for 2 h at room temperature. The resin was washed with DMF (3×1 min) and DCM (3×1 min) and the Fmoc-group removed with 20% piperidine as described above. The coupling procedure was repeated with additional Fmoc-Lys(N₃)—OH (3 equiv.) followed by washing with DMF (3×1 min) and DCM (3×1 min) and drying in vacuo to give 1.584 g resin loaded intermediate 2.5. The loading was determined as described in J. Pept. Sci. 2017, 23, 757-762 to result in a degree of loading 0.2 mmol/g.

A test cleavage was performed with 200 mg resin treaded two times with 1 mL DCM/TFA 95:5 for 15 min. The cleavage solutions were combined, evaporated and purified on prep. HPLC to result in 28 mg Fmoc protected diazide. HPLC (method AF): t_{R}=17.3 min. MS (ESI): found m/z=723.4 [M+H]⁺, calcd. m/z=723.4 [C₃₅H₅₁N₁₀O₇]⁺.

### Intermediate 2.6

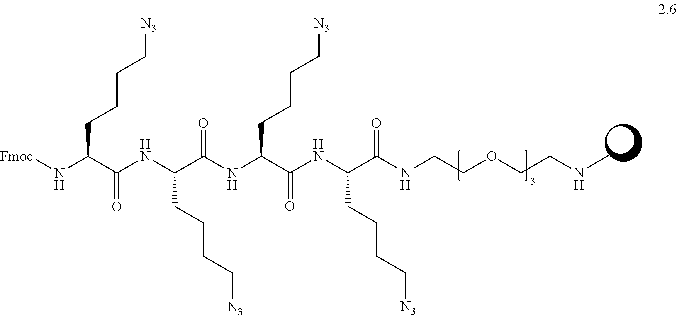

By repeating the procedure of intermediate 2.5 but with two additional coupling steps with Fmoc-Lys(N₃)—OH, resin loaded with intermediate 2.6 (1.626 g) was obtained with a degree of loading of 0.20 mmol/g.

A test cleavage was performed with 200 mg resin resulting in 48.36 mg Fmoc protected tetraazide. HPLC (method AF): t_{R}=19.9 min. MS (ESI): found m/z=1031.5 [M+H]⁺, calcd. m/z=1031.6 [C₄₇H₇₁N₁₈O₉]⁺.

### Intermediate 2.7

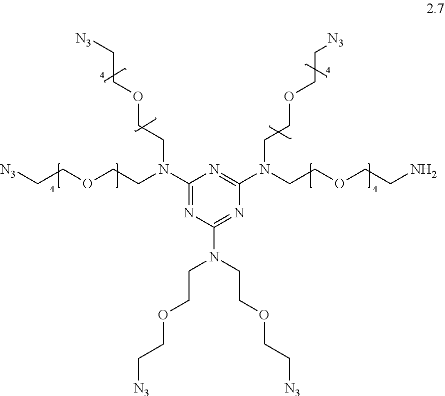

Intermediate 2.7 was synthesized in 7 steps:

### Step 1

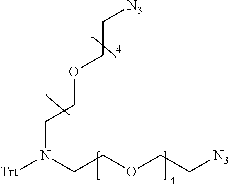

To a solution of 2,2′-(tritylazanediyl) diethanol (CAS Nr.: 23202-47-3, 1.89 g, 5.47 mmol, 1.0 equiv.) in 15 ml abs. DMF was added sodium hydride (60% dispersion in mineral oil, 656 mg, 3 equiv.) in portions and the mixture was stirred for 1 h until gas evolution ceased. A solution of azide-PEG4-Tos (CAS Nr.: 168640-82-2, 4.50 g, 12.0 mmol, 2.2 equiv.) in 5 ml DMF was added dropwise and the mixture was stirred overnight followed by 3 h at 50° C. Then the reaction was quenched with 50 ml 0.1M potassium phosphate buffer, pH 6 and the mixture was extracted with 75 ml ethyl acetate. The organic phase was washed with brine, dried (MgSO₄) and evaporated. The crude was purified on silica gel with a gradient from 0 to 50% acetone in hexane to result in 3.20 g (78% yield) step 1 product as a colorless oil. ¹H NMR (500 MHZ, CDCl₃) δ 7.55-7.45 (m, 5H), 7.26-7.20 (m, 5H), 7.17-7.10 (m, 3H), 3.67-3.58 (m, 28H), 3.55-3.50 (m, 4H), 3.37 (dd, J=5.5, 4.6 Hz, 4H), 2.54 (t, J=7.0 Hz, 4H).

¹³C NMR (126 MHZ, CDCl₃) δ 143.74, 129.31, 127.52, 125.99, 78.66, 71.20, 70.66, 70.63, 70.60, 70.52, 70.37, 70.00, 52.86, 50.64.

### Step 2

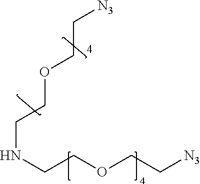

To step 1 product (2.16 g, 2.88 mmol) dissolved in 10 ml DCM/methanol 9:1 was added 500 μl formic acid and stirred for 5 min before 10 g diatomaceous earth was added and all volatiles were evaporated in vaccuo. The product was purified on silica gel with a gradient from 0 to 5% methanol in DCM with 0.5% ammonia as modifier to result in 1.325 g (91%) step 2 product as colorless oil. MS (ESI): found m/z=508.3 [M+H]⁺, calcd. m/z=508.3 [C₂₀H₄₂N₇O₈]⁺. ¹H NMR (500 MHZ, DMSO) δ 3.63-3.59 (m, 4H), 3.58-3.47 (m, 24H), 3.44 (t, J=5.6 Hz, 4H), 3.41-3.36 (m, 4H), 3.25 (s, 1H), 2.66 (t, J=5.7 Hz, 4H). ¹³C NMR (126 MHZ, DMSO) δ 70.08, 69.74, 69.71, 69.70, 69.68, 69.62, 69.54, 69.13, 49.94, 48.65.

### Step 3

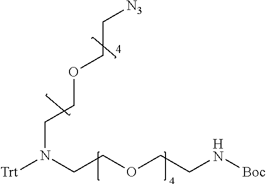

To a solution of step 1 product (1.015 g, 1.355 mmol, 1 equiv.) in 22.5 ml THF was added triphenyl phosphine (350 mg, 1 equiv.) and the mixture was stirred at room temperature until complete dissolution of the phosphine. Water (2.5 ml) was added subsequently, and the solution was stirred at room temperature for 3 days followed by addition of Diboc (591 mg, 2.71 mmol, 2 equiv.) and DIPEA (461 μl, 2.71 mmol, 2 equiv.) and stirring continued for 1 h, before diatomaceous earth was added, and the volatiles were evaporated, and the residue was dried thoroughly in high vacuum.

Purification on silica gel with a gradient from 0 to 37% acetone in hexane resulting in 502 mg (45%) step 3 product as colorless oil. MS (ESI): found m/z=824.0 [M+H]⁺, calcd. m/z=824.5 [C₄₄H₆₆N₅O₁₀]⁺. ¹H NMR (500 MHZ, CDCl₃) δ 7.52-7.47 (m, 6H), 7.23 (dd, J=8.3, 7.2 Hz, 6H), 7.17-7.10 (m, 3H), 5.03 (s, 1H), 3.69-3.58 (m, 26H), 3.56-3.49 (m, 6H), 3.39-3.34 (m, 2H), 3.30 (d, J=5.5 Hz, 2H), 2.54 (t, J=7.0 Hz, 4H), 1.44 (s, 9H). ¹³C NMR (126 MHZ, CDCl₃) δ 155.97, 143.73, 129.31, 127.53, 126.00, 79.12, 78.66, 71.20, 70.67, 70.63, 70.60, 70.55, 70.53, 70.49, 70.37, 70.21, 70.19, 70.01, 52.85, 50.64, 40.32, 28.41.

### Step 4

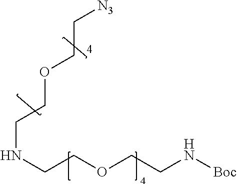

By applying the procedure of step 2, to step 3 product, 342 mg (91%) step 4 product were achieved as colorless oil. MS (ESI): found m/z=582.4 [M+H]⁺, calcd. m/z=582.4 [C₂₅H₅₂N₅O₁₀]⁺. ¹H NMR (500 MHZ, CDCl₃) δ 5.12 (s, 1H), 3.72-3.58 (m, 31H), 3.54 (t, J=5.2 Hz, 2H), 3.42-3.37 (m, 2H), 3.32 (q, J=5.4 Hz, 2H), 2.83 (t, J=5.4 Hz, 4H), 1.44 (s, 9H). ¹³C NMR (126 MHZ, CDCl₃) δ 156.00, 79.09, 70.67, 70.63, 70.60, 70.54, 70.50, 70.48, 70.32, 70.21, 70.01, 50.64, 49.14, 40.31, 28.40.

### Step 5

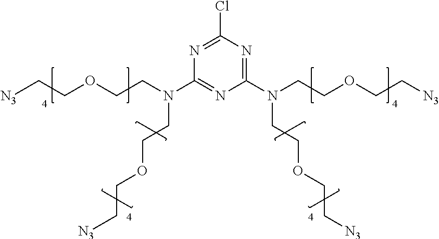

Cyanuric acid (86 mg, 0.467 mmol, 1.0 equiv.) and DIPEA (1.675 ml, 9.85 mmol, 21 eq.) were dissolved in 15 ml acetonitrile and the mixture was added to step 2 product (500 mg, 0.985 mmol, 2.1 eq.) and stirred at room temperature. After 3 h ethyl acetate was added, and acetonitrile was evaporated in vaccuo. The remaining aqueous phase was extracted two times with ethyl acetate (20 ml each) and the combined organic phases were washed with brine, dried (MgSO₄) and evaporated to dryness.

The crude was purified on silica gel with a gradient from 0 to 50% acetone in hexane to result in 403 mg (77%) step 5 product as colorless oil. MS (ESI): found m/z=1126.6 [M+H]⁺, calcd. m/z=1126.6 [C₄₃H₈₁ClN₁₇O₁₆]⁺. ¹H NMR (500 MHZ, CDCl₃) δ 3.78 (t, J=5.7 Hz, 4H), 3.73 (t, J=5.9 Hz, 4H), 3.69-3.56 (m, 64H), 3.44-3.34 (m, 8H). ¹³C NMR (126 MHZ, CDCl₃) δ 168.80, 164.53, 70.64, 70.63, 70.61, 70.58, 70.57, 70.55, 70.54, 70.51, 70.25, 70.00, 69.26, 68.88, 50.62, 48.12, 47.77.

### Step 6

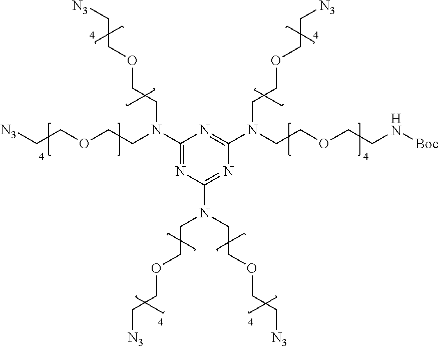

Step 6 product (145 mg, 129 μmol, 1.0 equiv.) and step 4 product (151 mg, 258 μmol, 2.0 equiv.) were dissolved in 4.5 ml acetonitrile, DIPEA (217 μl. 1.29 μmol, 10 eq.) was added and the mixture was heated to reflux for 3 d. After cooling, sufficient diatomaceous earth was added to soak all reaction mixture, and all volatiles were evaporated and the remaining was dried in high vacuum. The crude was purified on silica gel with a gradient from 0 to 50% acetone in hexane to result in 161 mg (75%) step 7 product as colorless oil. MS (ESI): found m/z=1671.9 [M+H]⁺, calcd. m/z=1672.0 [C₆₈H₁₃₁N₂₂O₂₆]⁺.

### Step 7

Step 6 product (57 mg, 34 μM) was dissolved in 250 μl DCM and 57 μl trifluoroacetic acid was added. The mixture was stirred at room temperature overnight bevor evaporation of the volatiles. The crude was purified by prep. HPLC to yield 40 mg (70%) of intermediate 2.7 as colorless oil. HPLC (method A): t_{R}=9.3 min. MS (ESI): found m/z=1571.9 [M+H]⁺, calcd. m/z=1571.9 [C₆₃H₁₂₃N₂₂O₂₄]⁺.

### Example 1.4—Synthesis of Intermediate 3.1

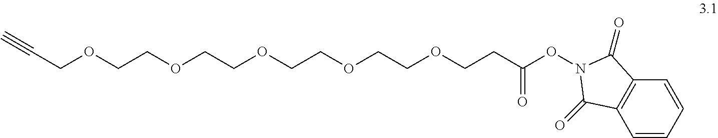 [SMILES: C#CCOCCOCCOCCOCCOCCC(=O)ON1C(=O)c2ccccc2C1=O]

EDC×HCl (485.8 mg, 2.53 mmol, 1.3 equiv.) and DIPEA (1.09 mL, 6.24 mmol, 3.2 equiv.) were added to a solution of Propargyl-PEG (5)-acid (CAS-Nr. 1245823-51-1, 593.1 mg, 1.94 mmol, 1 equiv.) in abs. THF (8.9 mL). A solution of N-hydroxyphthalimide (413.4 mg, 2.53 mmol, 1.3 equiv.) in abs. THF (8.9 mL) was added dropwise for 10 minutes. The reaction was stirred at room temperature and monitored by TLC and RP-HPLC. After 21 h, the solution was diluted with ethyl acetate (60 mL) and the organic layer washed with 1N HCl (30 mL), sodium bicarbonate solution (30 mL) and brine (30 mL). The organic layer was dried over magnesium sulfate and concentrated in vacuo. The resulting crude product (724.7 mg) was purified by flash chromatography on silica using a gradient from n-hexane to ethyl acetate. Intermediate 3.1 (457.3 mg, 52%) was obtained as yellowish oil. C₂₂H₂₇NO₉. ¹H NMR (500 MHZ, CDCl₃): δ (ppm)=7.89 (dd, J=5.5, 3.1 Hz, 2H), 7.80 (dd, J=5.5, 3.1 Hz, 2H), 4.21 (d, J=2.3 Hz, 2H), 3.89 (t, J=6.4 Hz, 2H), 3.73-3.63 (m, 17H), 2.97 (t, J=6.4 Hz, 2H), 2.44 (t, J=2.4 Hz, 1H). ¹³C NMR (126 MHZ, CDCl₃): δ (ppm)=167.65, 161.73, 134.73, 128.82, 123.92, 79.61, 74.48, 70.69, 70.58, 70.54, 70.53, 70.49, 70.46, 70.33, 69.04, 65.76, 58.33, 32.14. HPLC (method I): t_{R}=11.7 min. MS (ESI): found m/z=472.2 [M+Na]⁺, calcd. m/z=472.2 [M+Na]⁺.

### Example 1.5—Synthesis of Intermediate 3.2

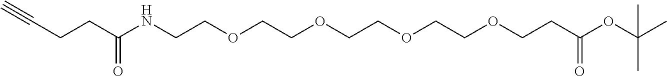 [SMILES: C#CCCC(=O)NCCOCCOCCOCCOCCC(=O)OC(C)(C)C]

To a solution of pent-4-ynoic acid (2 g, 20.4 mmol, 1.05 equiv.) in DMF (40 mL) were added HATU (11.64 g, 30.6 mmol, 1.6 equiv.) and DIPEA (10.7 mL, 61.2 mmol) at 5° C. and the resulting solution stirred for 10 min at 5° C. Then, a solution of tert-butyl 1-amino-3,6,9,12-tetraoxapentadecan-15-oate (6.24 g, 19.4 mmol, 1 equiv.) in DMF (10 mL) was added dropwise and the resulting solution allowed to warm to room temperature. After 2 h, the solution was concentrated in vacuo and the residue taken up in ethyl acetate (300 mL). The organic layer was washed with 0.2 M citric acid (150 mL), saturated sodium bicarbonate solution (150 mL) and brine (150 mL). The organic layer was dried over magnesium sulfate and concentrated in vacuo. Purification by flash chromatography on silica using a gradient from hexane to acetone afforded tert-butyl 1-(pent-4-ynamido)-3,6,9,12-tetraoxapentadecan-15-oate (8.20 g, quant.). C₂₀H₃₅NO₇. HPLC (method A): t_{R}=10.6 min. MS (ESI): found m/z=424.4 [M+Na]⁺, calcd. m/z=424.2 [M+Na]⁺.

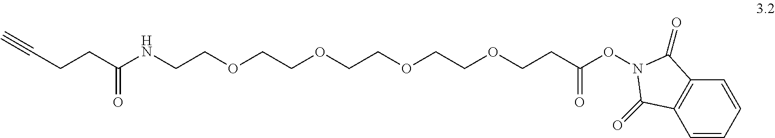 [SMILES: C#CCCC(=O)NCCOCCOCCOCCOCCC(=O)ON1C(=O)c2ccccc2C1=O]

tert-Butyl 1-(pent-4-ynamido)-3,6,9,12-tetraoxapentadecan-15-oate (8.20 g, max. 19.4 mmol, 1 equiv.) was dissolved in TFA (15.6 mL) and stirred for 1 h at 300 mbar. TFA was removed in vacuo and the residue co-evaporated twice with toluene. The residue obtained was dissolved in THE (abs., 50 ml) and N-Ethyl-N′-carbodiimide hydrochloride (4.66 g, 24.3 mmol, 1.25 equiv.) as well as DIPEA (11 mL, 63 mmol, 3.25 mmol) were added. Then, a solution of N-hydroxyphtalimide (3.96 g, 24.3 mmol, 1.25 equiv.) in THF (abs., 50 ml) was added dropwise and the resulting reddish solution stirred overnight at room temperature. Ethyl acetate (400 ml) was added and the organic layer washed with 2N HCl (100 ml), saturated sodium bicarbonate solution (100 mL) and brine (100 ml). The organic layer was dried over magnesium sulfate and concentrated in vacuo. Purification by flash chromatography on silica using a gradient from dichloromethane to acetone afforded intermediate 3.2 (4.96 g, 52%) as yellowish oil. C₂₄H₃₀N₂O₉. ¹H NMR (500 MHZ, DMSO-d6): δ (ppm)=8.01-7.89 (m, 4H), 7.87 (s, 1H), 3.77 (t, J=6.0 Hz, 2H), 3.63-3.46 (m, 12H), 3.40 (t, J=5.9 Hz, 2H), 3.20 (q, J=5.8 Hz, 2H), 3.01 (t, J=6.0 Hz, 2H), 2.70 (t, J=2.6 Hz, 1H), 2.39-2.31 (m, 2H), 2.31-2.23 (m, 2H). ¹³C NMR (126 MHZ, DMSO-d6): δ (ppm)=170.18, 168.21, 161.61, 135.39, 128.10, 123.85, 83.65, 71.00, 69.73, 69.68, 69.62, 69.60, 69.50, 69.01, 65.18, 38.50, 34.02, 31.55, 14.09. HPLC (method A): t_{R}=10.5 min. MS (ESI): found m/z=491.4 [M+H]⁺, calcd. m/z=491.2 [M+H]⁺.

### Example 1.6—Synthesis of Intermediates 4.x (N₃-PEGₙ-Beta/Gamma-CD

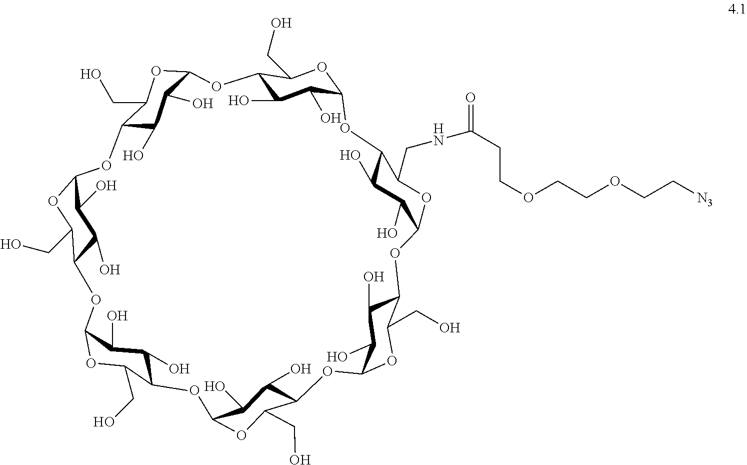 [SMILES: [N-]=[N+]=NCCOCCOCCC(=O)NCC1O[C@@H]2O[C@@H]3C(CO)O[C@H](O[C@@H]4C(CO)O[C@H](O[C@@H]5C(CO)O[C@H](O[C@@H]6C(CO)O[C@H](O[C@@H]7C(CO)O[C@H](O[C@@H]8C(CO)O[C@H](O[C@H]1[C@H](O)C2O)C(O)[C@H]8O)C(O)[C@H]7O)C(O)[C@H]6O)C(O)[C@H]5O)C(O)[C@H]4O)C(O)[C@H]3O]

Intermediate 4.1 was synthesized according to GP5 using azido-PEG (2)-NHS ester and 6-amino-6-deoxy-beta-cyclodextrin. The obtained crude product was used without further purification.

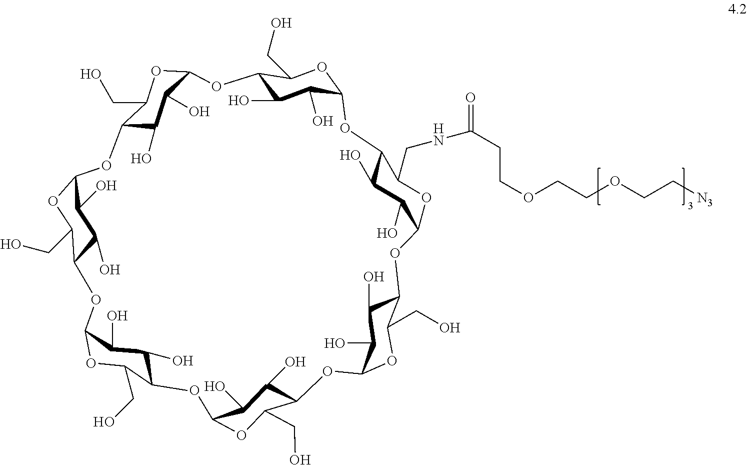

Intermediate 4.2 was synthesized according to GP5 using azido-PEG (4)-NHS ester and 6-amino-6-deoxy-beta-cyclodextrin. The obtained crude product was used without further purification.

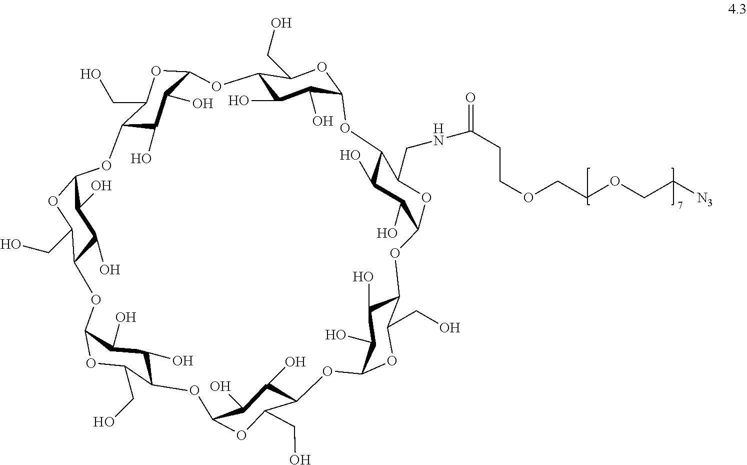

Intermediate 4.3 was synthesized according to GP5 using azido-PEG (8)-NHS ester and 6-amino-6-deoxy-beta-cyclodextrin. The obtained crude product was used without further purification.

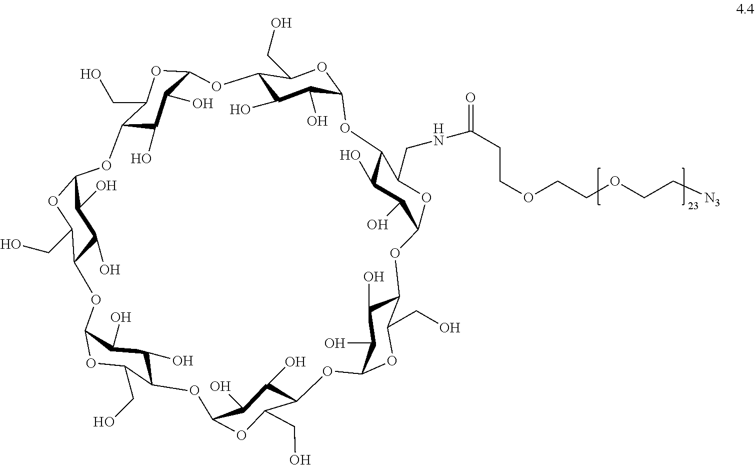

Intermediate 4.4 was synthesized according to GP5 using azido-PEG (24)-NHS ester and 6-amino-6-deoxy-beta-cyclodextrin. The obtained crude product was used without further purification.

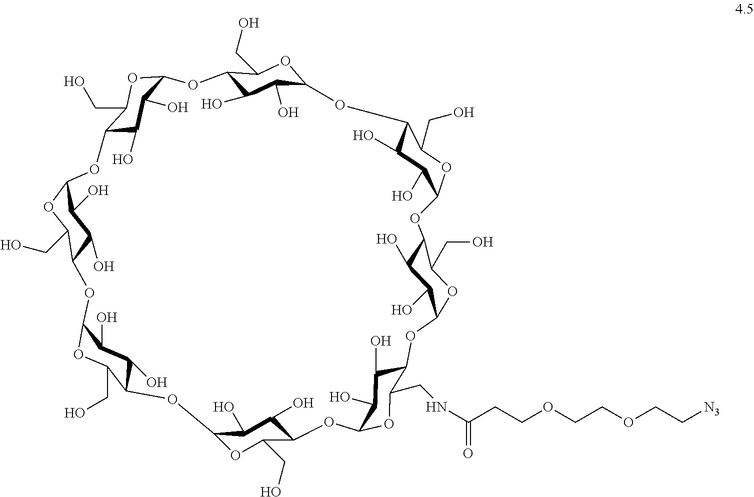 [SMILES: [N-]=[N+]=NCCOCCOCCC(=O)NCC1O[C@@H]2O[C@@H]3C(CO)O[C@@H](O[C@@H]4C(CO)O[C@H](O[C@@H]5C(CO)O[C@H](O[C@@H]6C(CO)O[C@H](O[C@@H]7C(CO)O[C@@H](O[C@@H]8C(CO)O[C@H](O[C@@H]9C(CO)O[C@H](O[C@H]1[C@H](O)C2O)C(O)[C@H]9O)C(O)[C@H]8O)C(O)[C@H]7O)C(O)[C@H]6O)C(O)[C@H]5O)C(O)[C@H]4O)C(O)[C@H]3O]

Intermediate 4.5 was synthesized according to GP5 using azido-PEG (2)-NHS ester and 6-amino-6-deoxy-gamma-cyclodextrin. The obtained crude product was used without further purification.

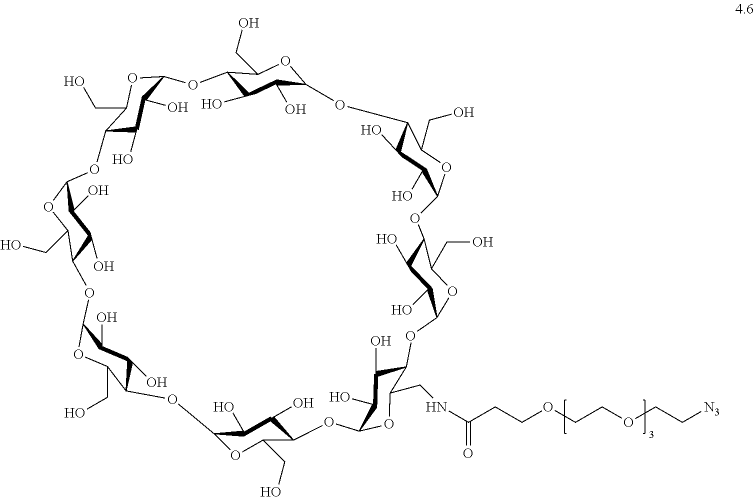

Intermediate 4.6 was synthesized according to GP5 using azido-PEG (4)-NHS ester and 6-amino-6-deoxy-gamma-cyclodextrin. The obtained crude product was used without further purification.

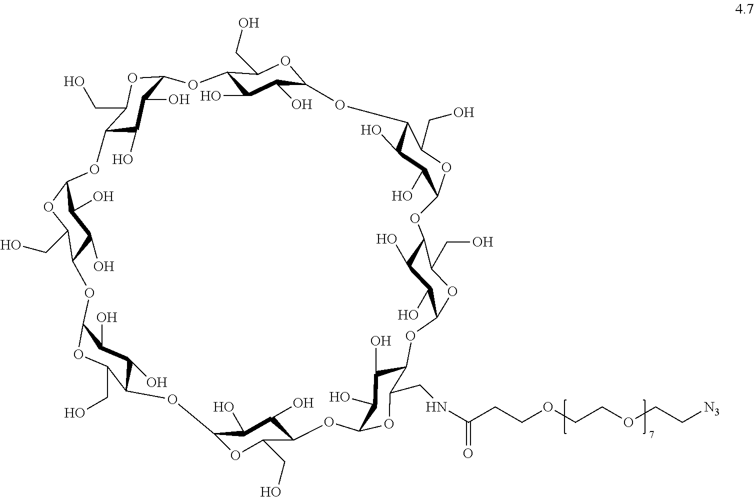

Intermediate 4.7 was synthesized according to GP5 using azido-PEG (8)-NHS ester and 6-amino-6-deoxy-gamma-cyclodextrin. The obtained crude product was used without further purification.

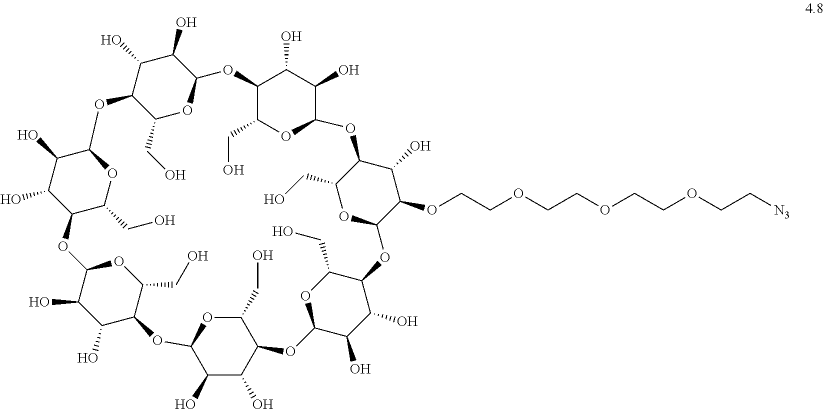 [SMILES: [N-]=[N+]=NCCOCCOCCOCCO[C@H]1[C@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@@H]([C@@H]1O)[C@@H](CO)O2)O[C@@H]8CO)O[C@@H]7CO)O[C@@H]6CO)O[C@@H]5CO)O[C@@H]4CO)O[C@@H]3CO]

Beta-cyclodextrin (2.000 g. 1.762 mmol) was co-evaporated twice with 30 mL dry DMSO (abs.) and re-dissolved in 30 mL DMSO. Lithium hydride (21 mg, 2.643 mmol, 1.5 equiv.) was added and the suspension was stirred under Argon atmosphere for 15 h. Subsequently, 1-azido-2-{2-[2-(2-bromoethoxy)ethoxy]ethoxy}ethane (497 mg, 1.762 mmol, 1.0 equiv.) dissolved in 2 mL DMSO (abs.) was added, followed by Lithium iodide (24 mg 176 μmol, 0.1 equiv.) and the mixture was stirred at 55° C. for 7.5 h and then at room temperature. After TLC (acetonitrile/water/conc. ammonia 6:3:1) indicated no further conversion, the mixture was concentrated to approximately 6 mL by in vacuo and the remaining was added dropwise to 160 mL ice cooled acetone. The resulting precipitate was isolated by centrifugation, resuspended in 120 mL cold acetone by sonification and centrifuged again. After drying in vacuo, the solid was taken up in 5 ml water, insoluble beta-cyclodextrin removed by filtration and the filtrate was freeze-dried. The crude product (2.263 g) was acidified to pH 2 with trifluoro acetic acid and purified by prep. RP-HPLC to result in 532 mg (23%) pure intermediate 4.8 as colorless lyophilisate. MS (ESI): found m/z=1336.8 [M+H]⁺, calcd. m/z=: 1336.5 [M+H]⁺.

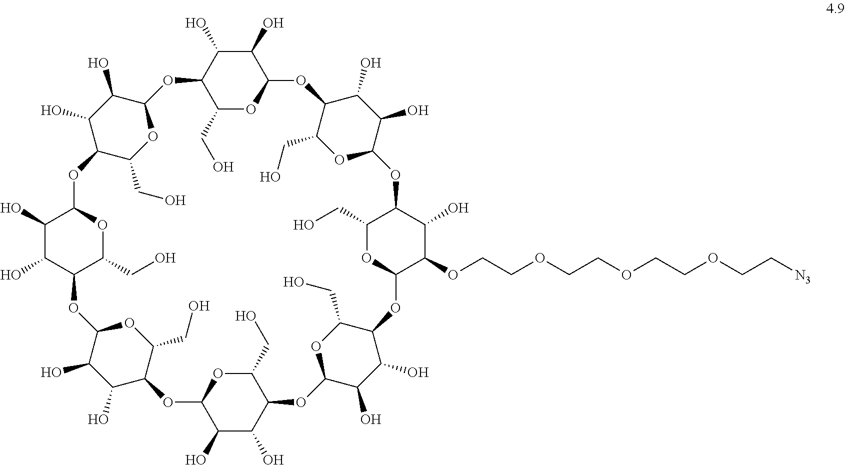 [SMILES: [N-]=[N+]=NCCOCCOCCOCCO[C@H]1[C@H]2O[C@H]3[C@H](O)[C@@H](O)[C@@H](O[C@H]4[C@H](O)[C@@H](O)[C@@H](O[C@H]5[C@H](O)[C@@H](O)[C@@H](O[C@H]6[C@H](O)[C@@H](O)[C@@H](O[C@H]7[C@H](O)[C@@H](O)[C@@H](O[C@H]8[C@H](O)[C@@H](O)[C@@H](O[C@H]9[C@H](O)[C@@H](O)[C@@H](O[C@@H]([C@@H]1O)[C@@H](CO)O2)O[C@@H]9CO)O[C@@H]8CO)O[C@@H]7CO)O[C@@H]6CO)O[C@@H]5CO)O[C@@H]4CO)O[C@@H]3CO]

Intermediate 4.9 was synthesized according to intermediate 4.8 using gamma-cyclodextrin. The target product was obtained in 22% yield as a colorless lyophilisate. MS (ESI): found m/z=1498.9 [M+H]⁺, calcd. m/z=1498.5 [M+H]⁺.

### Example 1.7—Synthesis of Intermediates 5.1 and 2

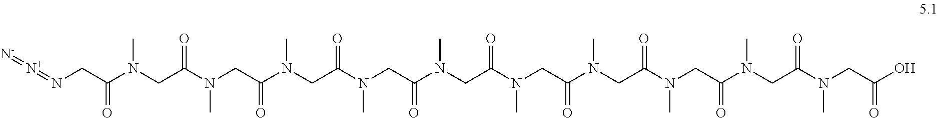 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN=[N+]=[N-]]

Intermediate 5.1 was synthesized according to WO2019081455 and WO2022207699 using Wang resin loaded with Fmoc-Sar, Fmoc-Sar-Sar-OH, Fmoc-Sar-OH and azidoacetic acid for N-terminal capping. Fmoc-deprotection was performed with 20% piperidine in DMF for 15 min (twice). Amino acid coupling was performed using amino acid (3 equiv.), HATU (2.9 equiv.) and DIPEA (6 equiv.) in DMF. Cleavage from resin was performed using neat TFA (10 mL/g resin) followed by precipitation from ice-cold methyl tert-butyl ether and purification by RP-HPLC. Intermediate 5.1 (166.7 mg, 41%) was obtained as colourless solid. C₃₂H₅₃N₁₃O₁₂. HPLC (method H): t_{R}=7.4 min. MS (ESI): found m/z=834.2 [M+Na]⁺, calcd. m/z=834.4 [M+Na]⁺.

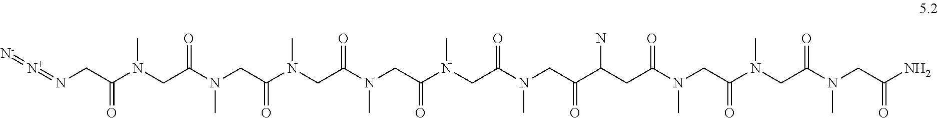 [SMILES: CN(CC(N)=O)C(=O)CN(C)C(=O)CN(C)C(=O)CC(N)C(=O)CN(C)C(=O)CN(C)C(=O)CN(C)C(=O)CN(C)C(=O)CN(C)C(=O)CN(C)C(=O)CN=[N+]=[N-]]

Intermediate 5.2 was synthesized according to WO2019081455 and WO2022207699 using NovaGel Rink-Amide resin loaded with Fmoc-Sar, Fmoc-Sar-Sar-OH, Fmoc-Sar-OH and azidoacetic acid for N-terminal capping. Fmoc-deprotection was performed with 20% piperidine in DMF for 15 min (twice). Amino acid coupling was performed using amino acid (3 equiv.), HATU (2.9 equiv.) and DIPEA (6 equiv.) in DMF. Cleavage from resin was performed using TFA/TIS 95/5 (10 mL/g resin) followed by precipitation from ice-cold methyl tert-butyl ether and purification by RP-HPLC. Intermediate 5.2 (83.6 mg, 54%) was obtained as colourless solid. C₃₂H₅₄N₁₄O₁₁. HPLC (method K): t_{R}=7.1 min. MS (ESI): found m/z=811.5 [M+H]⁺, calcd. m/z=811.4 [M+H]⁺.

### Example 1.8—Synthesis of Intermediates 6.1-4

### Synthesis of Intermediate 6.1

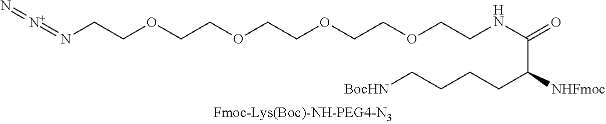

To a solution of Fmoc-Lys(Boc)-OH (3.056 g, 6.5 mmol, 1 equiv.) in DMF (30 mL) were added TBTU (2.467 g, 7.8 mmol, 1.2 equiv) and DIPEA (1.33 mL, 7.8 mmol, 1.2 equiv.) and the resulting solution mixed for 1 min. Then, the pre-activated amino acid solution was added to a solution of 14-azido-3,6,9,12-tetraoxatetradecan-1-amine (1.680 g, 6.5 mmol, 1 equiv.) in DMF (10 mL) and the resulting solution stirred for 2 h at room temperature. Water (5 mL) was added and the solution evaporated in vacuo. The residue was dissolved in ethyl acetate (100 mL) and the organic layer washed with aqueous 0.2 M citric acid solution (50 mL), aqueous saturated sodium bicarbonate solution (50 mL) and brine (50 mL). The organic layer was dried over magnesium sulfate and evaporated in vacuo. Purification by flash chromatography on silica using a gradient from MTBE to acetone afforded Fmoc-Lys(Boc)-NH-PEG4-N₃ (3.545 g, 76%) as colourless, sticky solid. C₃₆H₅₂N₆O₉. ¹H NMR (500 MHZ, DMSO-d6): δ (ppm)=7.88 (dt, J=7.7, 0.9 Hz, 2H), 7.82 (t, J=5.7 Hz, 1H), 7.76-7.68 (m, 2H), 7.42 (td, J=7.5, 1.0 Hz, 2H), 7.33 (tt, J=7.4, 1.2 Hz, 3H), 6.68 (s, 1H), 4.30-4.18 (m, 3H), 3.93 (td, J=8.6, 5.2 Hz, 1H), 3.62-3.56 (m, 2H), 3.55-3.48 (m, 12H), 3.41 (t, J=6.0 Hz, 2H), 3.37 (dd, J=5.6, 4.4 Hz, 2H), 3.21 (ddt, J=19.6, 13.6, 7.6 Hz, 2H), 2.89 (q, J=10.3, 8.3 Hz, 2H), 1.69-1.43 (m, 2H), 1.37 (s, 9H), 1.24 (s, 4H). ¹³C NMR (126 MHz, DMSO-d6): δ (ppm)=171.86, 155.78, 155.45, 143.79, 143.70, 140.60, 127.49, 126.92, 125.17, 119.95, 77.21, 69.71, 69.69, 69.67, 69.63, 69.60, 69.48, 69.11, 68.88, 65.50, 54.56, 49.93, 46.62, 38.47, 31.63, 29.08, 28.16, 22.69. HPLC (method A): t_{R}=14.3 min. MS (ESI): found m/z=735.4 [M+Na]⁺, calcd. m/z=735.4 [M+Na]⁺.

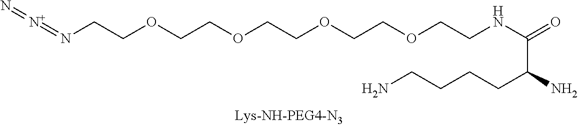 [SMILES: N=[N+]=NCCOCCOCCOCCOCCNC(=O)[C@@H](N)CCCCN]

To a solution Fmoc-Lys(Boc)-NH-PEG4-N₃ (2.00 g, 2.8 mmol, 1 equiv.) in DMF (20 mL) was added diethylamine (5.00 mL, 56.1 mmol, 17 equiv.) and the resulting solution stirred for 30 min at 600 mbar at room temperature. The solvent was removed in vacuo and purification by flash chromatography on silica using a gradient from dichloromethane to dichloromethane/MeOH/conc. ammonia (9:1:0.1) afforded the Fmoc-deprotected intermediate H-Lys(Boc)-NH-PEG4-N₃ (1.182 g, 86%) as colourless oil. As the next step, to a solution of H-Lys(Boc)-NH-PEG4-N₃ (1.163 g, 2.4 mmol, 1 equiv.) in 1,4-dioxane (20 mL) was added 1M HCl (11.85 mL, 11.8 mmol, 5 equiv.) and stirred for 10 min at 600 mbar at room temperature. The solvent was removed in vacuo, the residue dissolved in 1M HCl (11.85 mL, 11.8 mmol, 5 equiv.) and stirred for 10 min at 600 mbar at room temperature. The solvent was removed in vacuo and additionally two times co-evaporated with 1,4-dioxane. Lys-NH-PEG4-N₃ (1.119 g, quant.) was obtained as highly viscous, colorless oil. C₁₆H₃₄N₆O₅. ¹H NMR (500 MHZ, DMSO-d6): δ (ppm)=8.67 (t, J=5.6 Hz, 1H), 8.32 (s, 3H), 8.11 (s, 3H), 3.78-3.72 (m, 1H), 3.61-3.58 (m, 2H), 3.59-3.47 (m, 12H), 3.45 (t, J=5.9 Hz, 2H), 3.38 (dd, J=5.5, 4.4 Hz, 2H), 3.28 (d, J=5.9 Hz, 1H), 3.23 (dq, J=13.7, 5.8 Hz, 1H), 2.73 (d, J=8.7 Hz, 2H), 1.73 (q, J=7.3 Hz, 2H), 1.59 (qd, J=8.5, 7.8, 6.0 Hz, 2H), 1.42-1.33 (m, 2H). ¹³C NMR (126 MHZ, DMSO-d6): δ (ppm)=168.43, 69.74, 69.70, 69.66, 69.62, 69.49, 69.13, 68.63, 51.78, 49.97, 38.66, 38.11, 30.18, 26.08, 20.97. HPLC (method H): t_{R}=6.9 min. MS (ESI): found m/z=391.3 [M+H]⁺, calcd. m/z=391.3 [M+H]⁺.

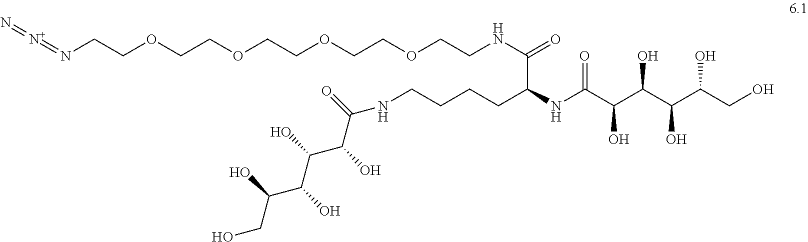 [SMILES: N=[N+]=NCCOCCOCCOCCOCCNC(=O)[C@H](CCCCNC(=O)[C@H](O)[C@@H](O)[C@H](O)[C@H](O)CO)NC(=O)[C@H](O)[C@@H](O)[C@H](O)[C@H](O)CO]

To a solution of Lys-NH-PEG4-N₃ (500 mg, 1.1 mmol, 1 equiv.) in DMF (10 mL) were added D-Glucono-1,5-lactone (481 mg, 2.7 mmol, 2.5 equiv.) and DIPEA (917 μL, 5.4 mmol, 5.0 equiv.) and the resulting solution stirred for 15 h at room temperature followed by 1 h at 80° C. After 16.5 h reaction time in total, D-Glucono-1,5-lactone (481 mg, 2.7 mmol, 2.5 equiv.) and DIPEA (917 μL, 5.4 mmol, 5.0 equiv.). After 21 h reaction time in total, the solvent was removed in vacuo and the residue co-evaporated with a mixture of methanol (10 mL) and DIPEA (917 μL) followed by co-evaporation with methanol (10 mL). The crude product was precipitated from ice-cold MTBE. Purification by preparative HPLC afforded intermediate 6.1 (294 mg, 36%) as colorless, glass-like solid. C₂₈H₅₄N₆O₁₇. ¹H NMR (500 MHZ, DMSO-d6): δ (ppm)=7.90 (t, J=5.7 Hz, 1H), 7.61 (d, J=8.4 Hz, 1H), 7.53 (t, J=5.9 Hz, 1H), 4.41 (s, 10H), 4.22 (td, J=8.4, 5.0 Hz, 2H), 4.06 (d, J=3.5 Hz, 1H), 3.97 (d, J=3.7 Hz, 1H), 3.93-3.86 (m, 3H), 3.62-3.56 (m, 4H), 3.56-3.43 (m, 14H), 3.43-3.35 (m, 4H), 3.20 (qd, J=6.0, 3.5 Hz, 4H), 3.05 (dp, J=13.9, 6.9 Hz, 2H), 1.73-1.64 (m, 1H), 1.56 (tdd, J=13.5, 9.4, 5.3 Hz, 1H), 1.45-1.33 (m, 2H), 1.23 (s, 2H). ¹³C NMR (126 MHZ, DMSO-d6): δ (ppm)=172.28, 172.17, 171.28, 73.50, 73.32, 72.36, 72.16, 71.46, 70.42, 70.10, 69.72, 69.64, 69.50, 69.14, 68.75, 63.29, 63.24, 51.99, 49.97, 38.52, 38.08, 31.75, 28.77, 22.46. HPLC (method M): t_{R}=13.5 min. MS (ESI): found m/z=747.4 [M+H]⁺, calcd. m/z=747.4 [M+H]⁺.

### Synthesis of Intermediate 6.2

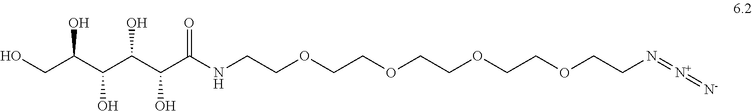 [SMILES: [N-]=[N+]=NCCOCCOCCOCCOCCNC(=O)[C@H](O)[C@@H](O)[C@H](O)[C@H](O)CO]

14-Azido-3,6,9,12-tetraoxatetradecan-1-amine (262 mg, 1.0 mmol, 1 equiv.) was dissolved in DMF (2 mL). D-Glucono-1,5-lactone (214 mg, 1.2 mmol, 1.2 equiv.) was added as solid followed by the addition of DIPEA (348 μL, 2.0 mmol, 2 equiv.) and the resulting solution stirred for 2 h at 110° C. After cooling to room temperature, the solvent was evaporated in vacuo. Purification by flash chromatography on silica using a gradient from dichloromethane to dichloromethane/methanol/water (5:1:0.1) afforded intermediate 6.1 (318 mg, 72%). C₁₆H₃₂N₄O₁₀. HPLC (method M): t_{R}=13.7 min. MS (ESI): found m/z=441.3 [M+H]⁺, calcd. m/z=441.2 [M+H]⁺.

### Synthesis of Intermediate 6.3

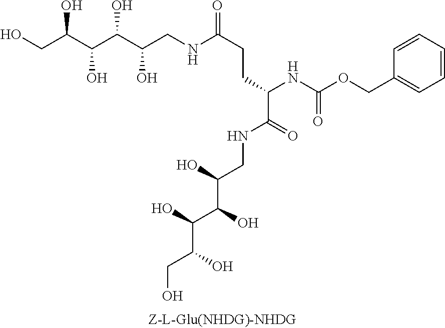 [SMILES: O=C(CC[C@H](NC(=O)OCc1ccccc1)C(=O)NC[C@H](O)[C@@H](O)[C@H](O)[C@H](O)CO)NC[C@H](O)[C@@H](O)[C@H](O)[C@H](O)CO]

To a solution of Z-L-Glu-OH (2.50 g, 8.9 mmol, 1 equiv.) in THF (abs., 50 mL) was added N-hydroxy-succinimid (2.32 g, 20.2 mmol, 2.3 equiv.) and the solution cooled to 0° C. Then, N,N′-dicyclohexylcarbodiimide (3.85 g, 18.7 mmol, 2.1 equiv.) was added and the resulting suspension was stirred 2 h at 0° C. followed by stirring overnight at room temperature. For work-up, the formed urea was removed by filtration and the oily residue was dissolved in ethyl acetate (75 mL). The organic layer was washed with saturated aqueous sodium bicarbonate solution, brine and water, dried over magnesium sulfate and the solvent removed in vacuo. After treatment with diethyl ether, Z-L-Glu(OSu)-OSu (3.68 g, 87%) was obtained as intermediate, which was used without further purification.

Next, to a solution of Z-L-Glu(OSu)-OSu (1.00 g, 2.1 mmol, 1 equiv.) in DMF (abs., 40 mL) was added D-glucamine (762.23 mg, 4.2 mmol, 2 equiv.) and the resulting suspension stirred for 3 h at 65° C. As the reaction progressed, the initial suspension became a solution. The solution was removed in vacuo and purification by RP-HPLC afforded Z-L-Glu (NHDG)-NHDG (933.9 mg, 73%). C₂₅H₄₁N₃O₁₄. HPLC (method H): t_{R}=8.0 min. MS (ESI): found m/z=630.3 [M+Na]⁺, calcd. m/z=630.2 [M+Na]⁺.

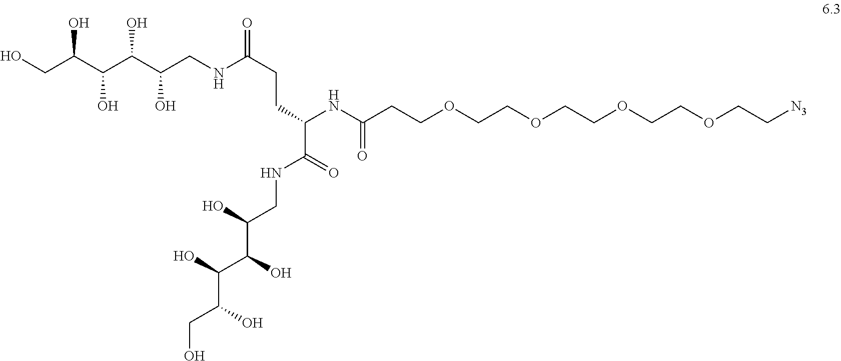 [SMILES: [N-]=[N+]=NCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO]

To a solution of Z-L-Glu (NHDG)-NHDG (924.9 mg, 1.5 mmol) in methanol (180 mL) was added Pd/C (10%, 161 mg) under Argon. The atmosphere was changed from argon to hydrogen, and the resulting suspension was stirred for 3.5 h at room temperature. The catalyst was removed by filtration through Celite and the solvent of the resulting filtrate was removed in vacuo affording H-L-Glu (NHDG)-NHDG (728.7 mg, 1.5 mmol, quant.). Next, H-L-Glu (NHDG)-NHDG (100.0 mg, 211.2 μmol, 1.5 equiv.) was dissolved in DMF (abs., 2 mL) and added to a solution of azido-PEG4-NHS ester (CAS-Nr. 944251-24-5, 54.68 mg, 140.8 μmol, 1 equiv.) under an atmosphere of argon. DIPEA (73.57 μL, 422.4 μmol, 3 equiv) was added and the resulting solution stirred for 1.5 h at room temperature. The solvent was removed in vacuo and purification by RP-HPLC afforded intermediate 6.3 (82.74 mg, 79%). C₂₈H₅₄N₆O₁₇. HPLC (method A): t_{R}=5.9 min. MS (ESI): found m/z=747.2 [M+H]⁺, calcd. m/z=747.4 [M+H]⁺.

### Synthesis of Intermediate 6.4

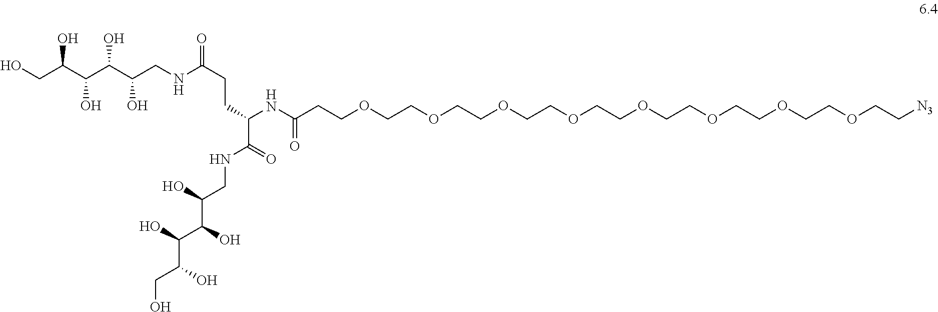 [SMILES: [N-]=[N+]=NCCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO]

Intermediate 6.4 (91.9 mg, 71%) was synthesized as described for intermediate 6.3 but using azido-PEG8-NHS ester (CAS-Nr. 1204834-00-3) instead of azido-PEG4-NHS ester. C₃₆H₇₀N₆O₂₁. HPLC (method A): t_{R}=6.7 min. MS (ESI): found m/z=923.6 [M+H]⁺, calcd. m/z=923.5 [M+H]⁺.

### Example 2—Synthesis of Multimeric Linker IV

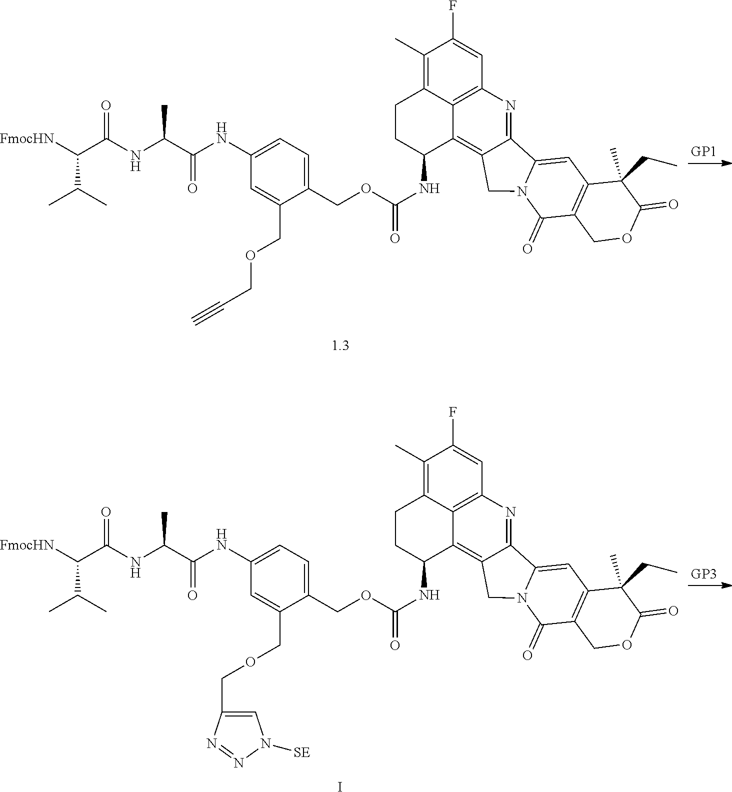

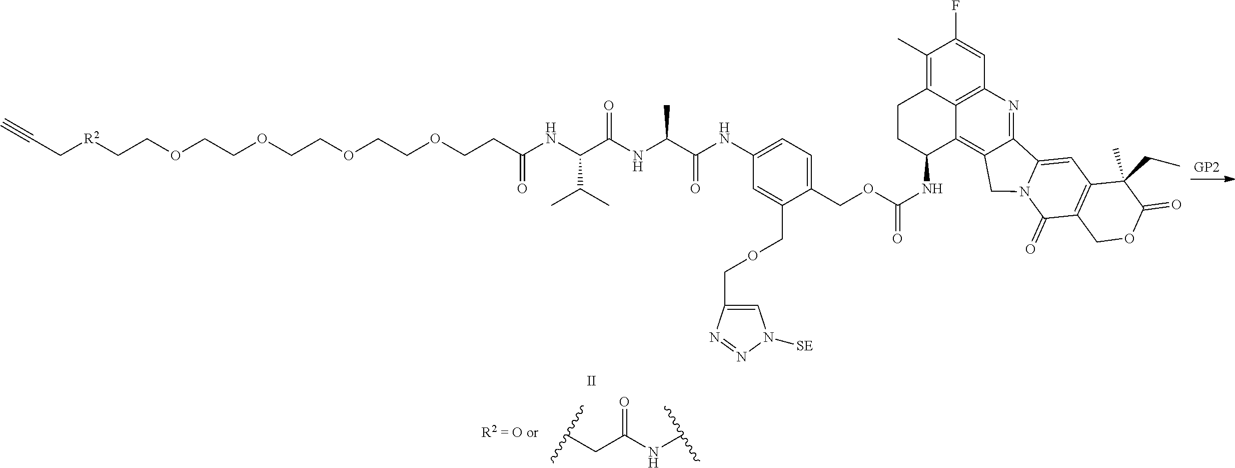

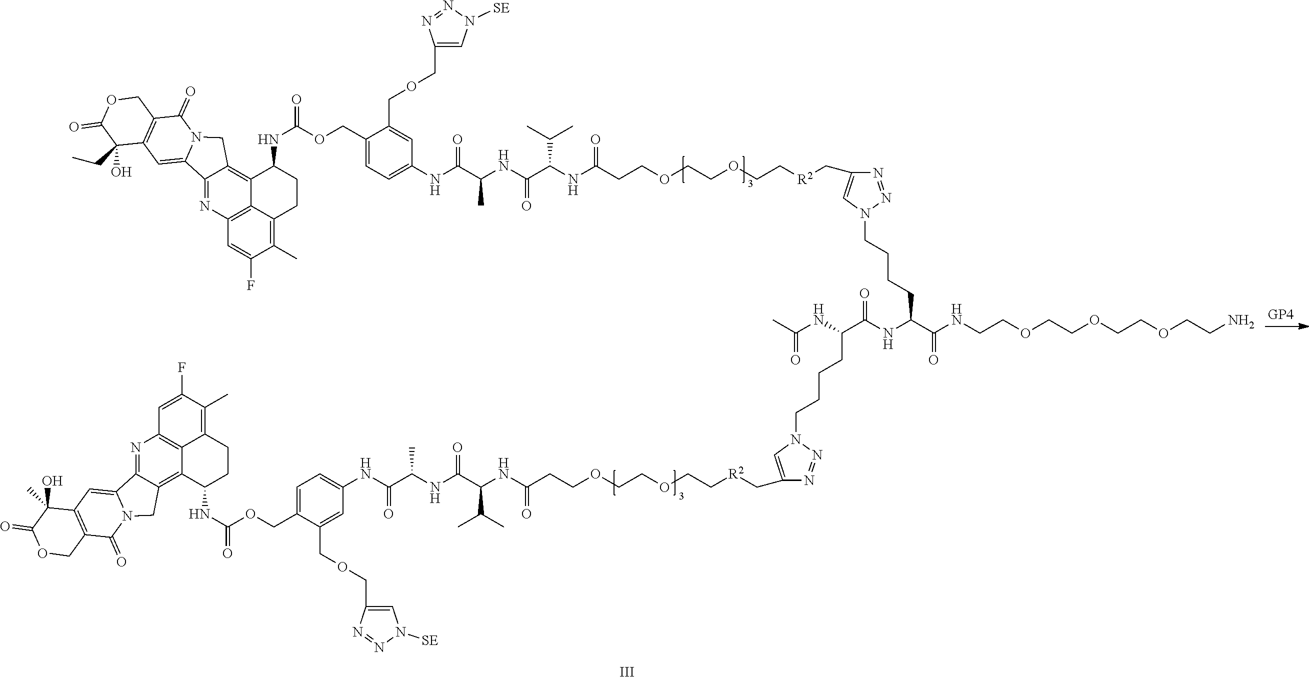

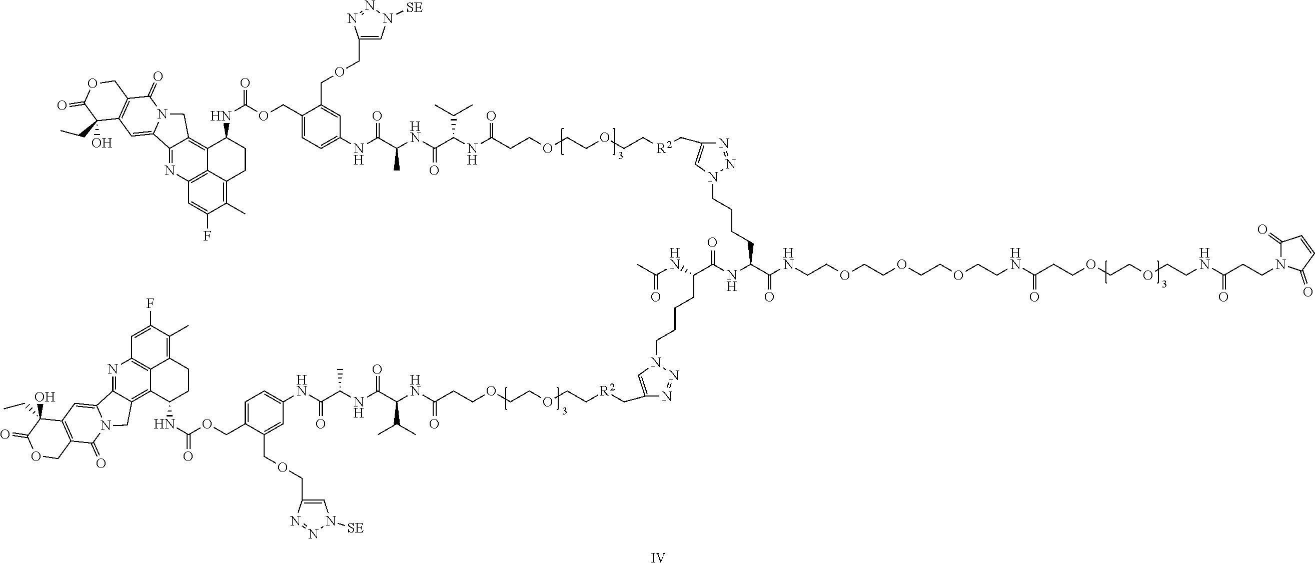

### Example 3—Synthesis of Compound IV.1

### Synthesis of I.1

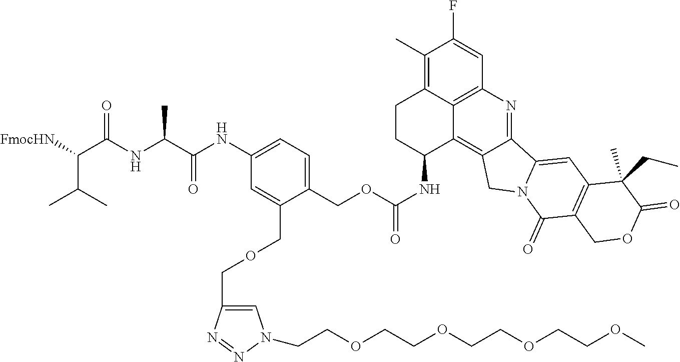

Intermediate I.1 was synthesized according to GP1 using m-PEG (4)-azide (40.4 mg, 173 μmol, 1.2 equiv.), intermediate 1.3 (150 mg, 144 μmol, 1.0 equiv.), copper (II) sulfate pentahydrate (3.6 mg, 14.4 μmol, 0.1 equiv.), tris(benzyltriazolylmethyl)amine (9.6 mg, 18 μmol, 0.125 equiv.) and sodium ascorbate (7.1 mg, 36 μmol, 0.25 equiv.) in DMF/water (1.65 mL, 10:1). Purification by RP-HPLC afforded intermediate I.1 (137 mg, 74%) as yellowish solid. HPLC (method D): t_{R}=11.8 min. MS (ESI): found m/z=1278.5 [M+H]⁺, calcd. m/z=1278.5 [M+H]⁺.

### Synthesis of II.1

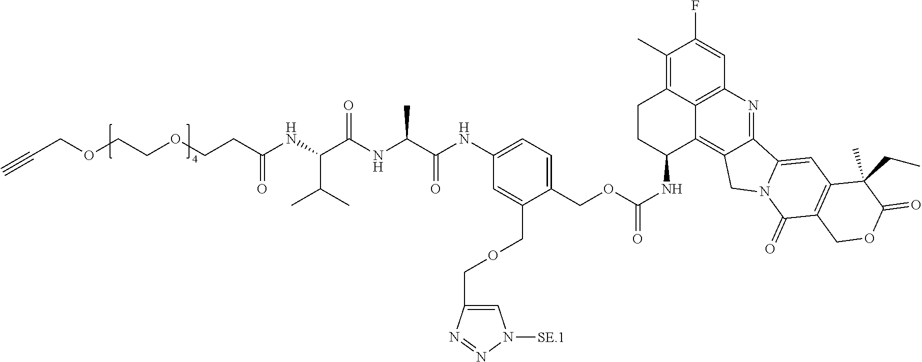

Intermediate II.1 was synthesized according to GP3 using 222 μL diethylamine (2.14 mmol, 20 equiv.) in 1.37 mL DMF for Fmoc-deprotection and intermediate I.1 (137 mg, 107 μmol, 1 equiv.), intermediate 3.1 (53.0 mg, 118 μmol, 1.1 equiv.), diisopropylethylamine (56 μL, 321 μmol, 3 equiv.) in DMF (1 mL) for the coupling reaction. Purification by RP-HPLC afforded intermediate II.1 (101 mg, 70%) as yellowish solid. HPLC (method A): t_{R}=11.1 min. MS (ESI): found m/z=1342.5 [M+H]⁺, calcd. m/z=1342.6 [M+H]⁺.

### Synthesis of III.1

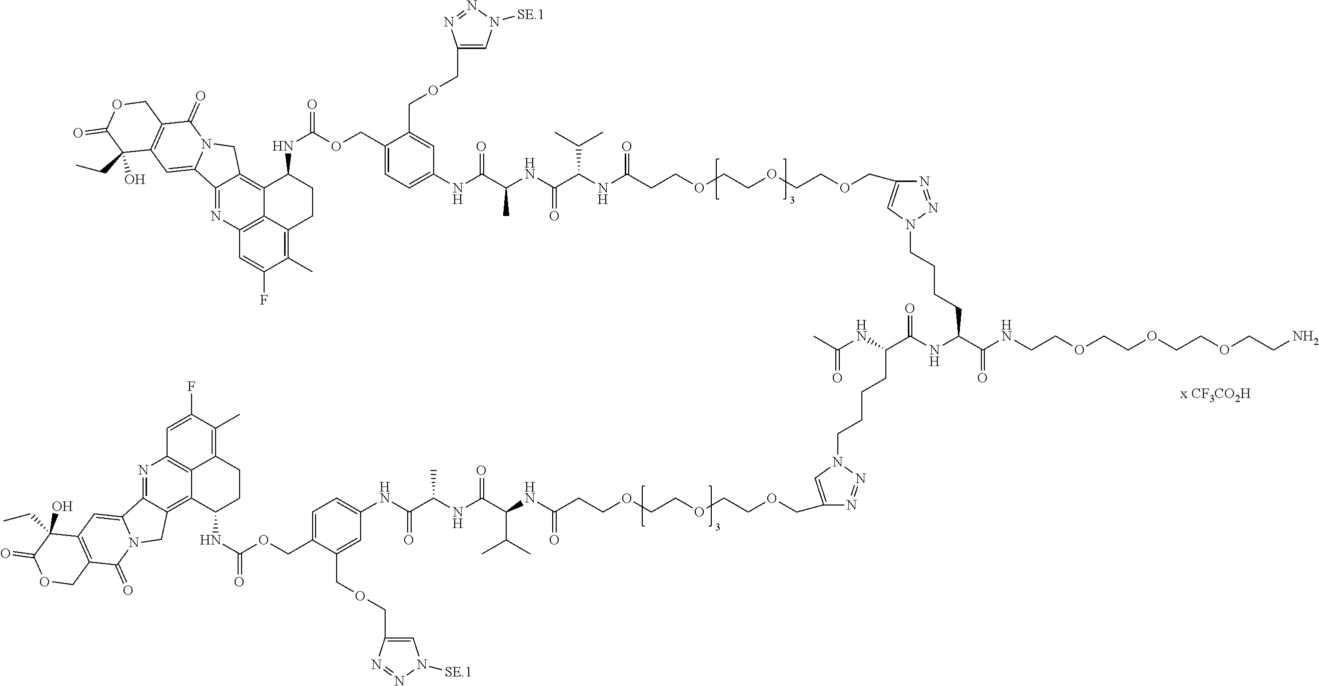

Intermediate III. 1 was synthesized according to GP2 using intermediate 2.1 (3.3 mg, 5.0 μmol, 1 equiv.), intermediate II.1 (17.3 mg, 12.9 μmol, 2.5 equiv.), copper (II) sulfate pentahydrate (1.6 mg, 6.5 μmol, 1.25 equiv.), Tris(3-hydroxypropyltriazolylmethyl)amine (5.6 mg, 12.9 μmol, 2.5 equiv.) and sodium ascorbate (4.2 mg, 21.4 μmol, 4.3 equiv.) in DMF/water (0.55 mL, 10:1). Purification by RP-HPLC afforded intermediate III.1 (22.4 mg, quant.) as yellowish solid. HPLC (method J): t_{R}=15.0 min. MS (ESI): found m/z=1614.17 [M+2H]²⁺, calcd. m/z=1613.8 [M+2H]²⁺.

### Synthesis of IV.1

Compound IV.1 was synthesized according to GP4 using the TFA-salt of intermediate III. 1 (16.3 mg, 4.9 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (10.8 mg, 21.0 μmol, 4.3 equiv.) and DIPEA (3.3 μL, 19.0 μmol, 3.9 equiv.) in DMF (652 μL). Purification by RP-HPLC afforded compound IV.1 (7.4 mg, 42%) as yellowish solid. C₁₇₄H₂₄F₂N₃₀O₅₂. HPLC (method): t_{R}=16.7 min. MS (ESI): found m/z=1209.6 [M+3H]³⁺, calcd. m/z=1208.9 [M+3H]³⁺.

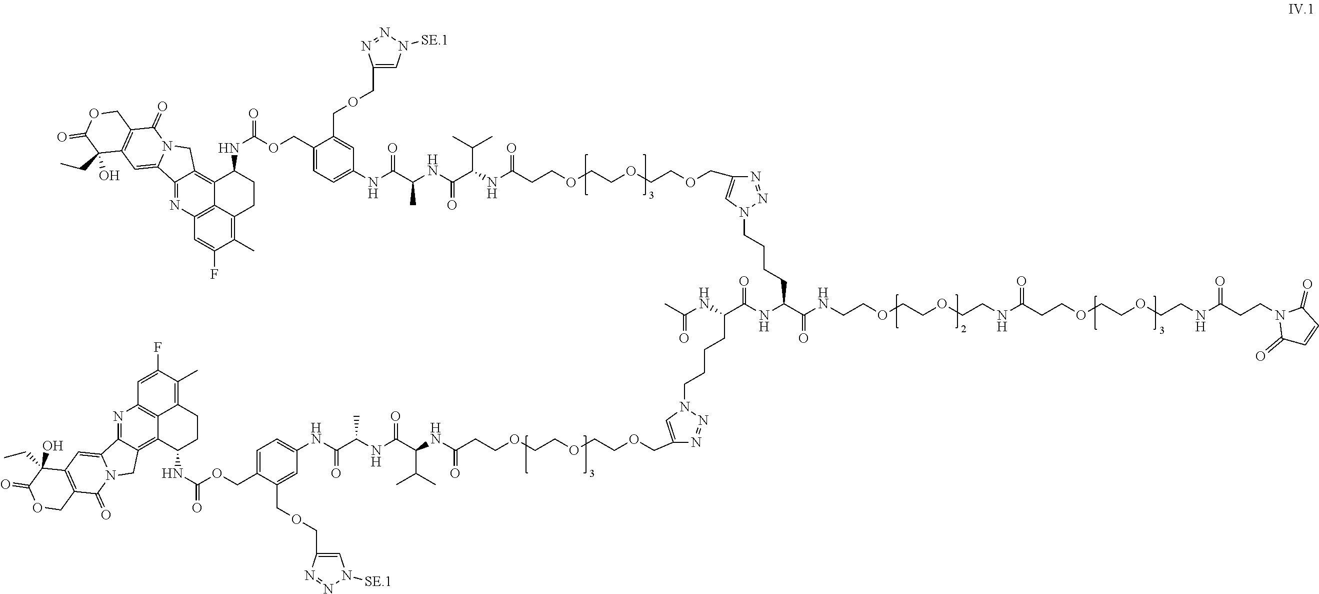

### Example 4—Synthesis of Compound IV.2

Compound IV.2 was synthesized as described for compound IV.1 but using intermediate 3.2. Compound IV.2 (30.76 mg) was obtained as yellowish solid. C₁₇₈H₂₅₀F₂N₃₂O₅₂. HPLC (method J): t_{R}=16.4 min. MS (ESI): found m/z=1236.8 [M+3H]³⁺, calcd. m/z=1236.3 [M+3H]³⁺.

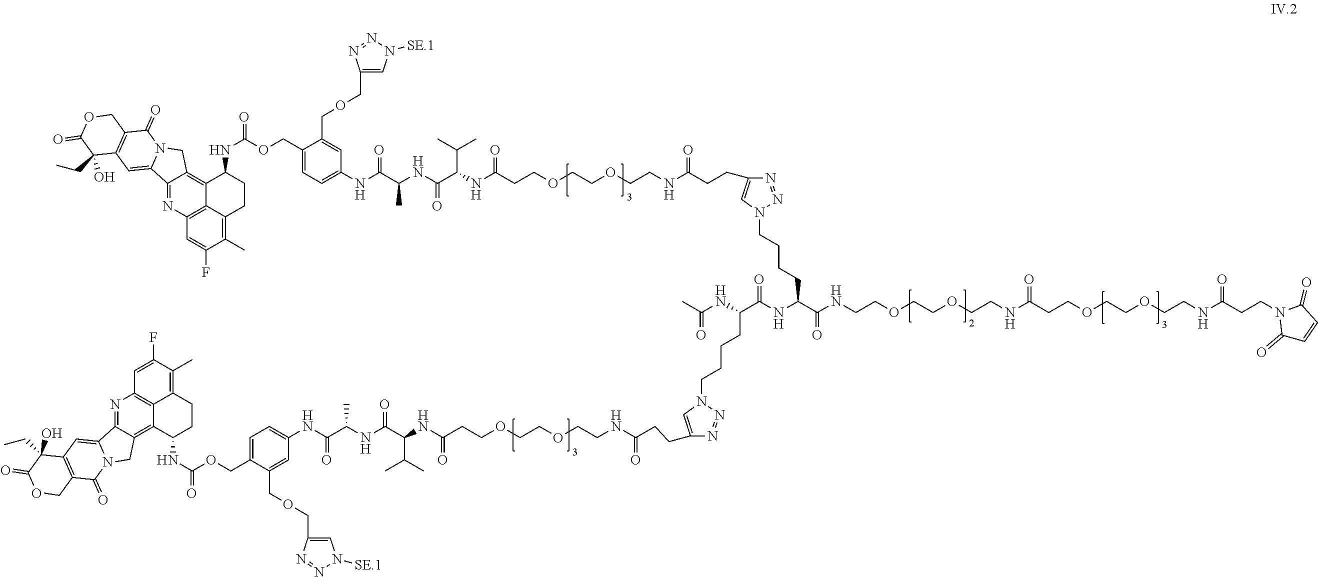

### Example 5—Synthesis of Compound IV.3

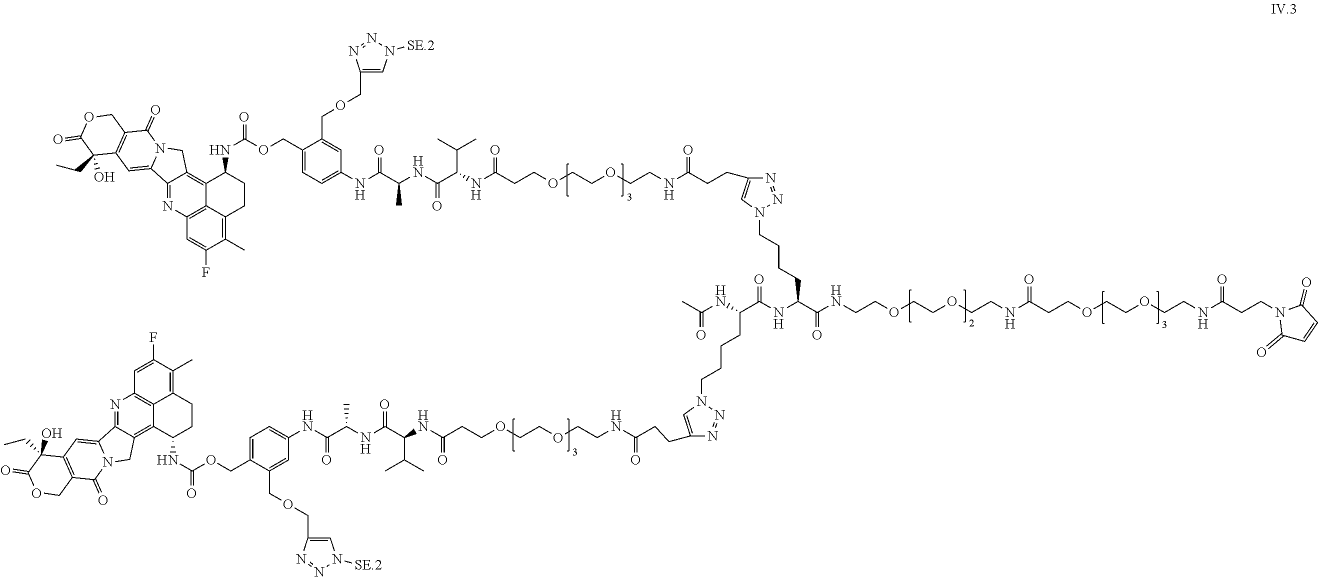

Compound IV.3 was synthesized as described for compound IV.1 but using m-PEG (12)-azide instead of m-PEG (4)-azide and intermediate 3.2 instead of intermediate 3.1. Compound IV.3 (5.07 mg) was obtained as yellowish solid. C₂₁₀H₃₁₄F₂N₃₂O₆₈. HPLC (method J): t_{R}=16.4 min. MS (ESI): found m/z=1471.7 [M+3H]³⁺, calcd. m/z=1471.1 [M+3H]³⁺.

### Example 6—Synthesis of Compound IV.4

### Synthesis of I.4

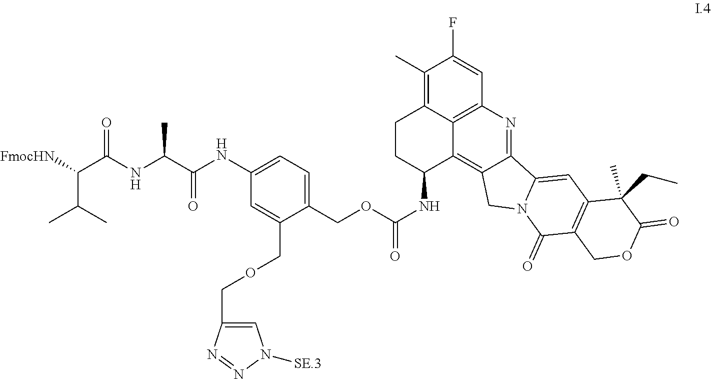

Intermediate I.4 was synthesized according to GP1 using intermediate 4.1 (22.4 μmol, 1 equiv.), intermediate 1.3 (26.16 mg, 25.0 μmol, 1.1 equiv.), copper (II) sulfate pentahydrate (1.23 mg, 4.9 μmol, 0.2 equiv.), tris(benzyltriazolylmethyl)amine (3.17 mg, 6.0 μmol, 0.26 equiv.) and sodium ascorbate (4.33 mg, 11.9 μmol, 0.5 equiv.) in DMF/water (0.28 mL, 10:1). Purification by RP-HPLC afforded intermediate 1.4 (31.51 mg, 60%) as yellowish solid. C₁₀₈H₁₃₉FN₁₀O₄₈. HPLC (method D): t_{R}=10.7 min. MS (ESI): found m/z=1182.5 [M+2H]²⁺, calcd. m/z=1182.4 [M+2H]²⁺.

### Synthesis of II.4

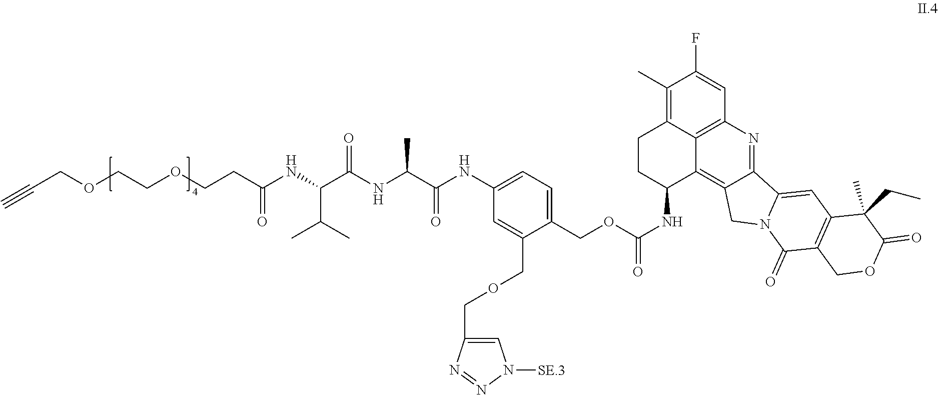

Intermediate II.4 was synthesized according to GP3 using intermediate 1.4 (31.51 mg, 13.3 μmol, 1 equiv.), 33 μL diethylamine (319.9 μmol, 24 equiv.) in 1.6 mL DMF for Fmoc-deprotection and intermediate 3.1 (8.98 mg, 20.0 μmol, 1.5 equiv.), diisopropylethylamine (6.8 μL, 40.0 μmol, 3 equiv.) in DMF (2.1 mL) for the coupling reaction. Purification by RP-HPLC afforded intermediate II.4 (15.51 mg, 50%) as yellowish solid. C₁₀₇H₁₅₁FN₁₀O₅₂. HPLC (method D): t_{R}=9.1 min. MS (ESI): found m/z=1214.5 [M+2H]²⁺, calcd. m/z=1214.5 [M+2H]²⁺.

### Synthesis of III.4

Intermediate III.4 was synthesized according to GP2 using intermediate 2.1 (1.54 mg, 2.6 μmol, 1 equiv.), intermediate II.4 (15.51 mg, 6.4 μmol, 2.5 equiv), copper (II) sulfate pentahydrate (0.87 mg, 3.5 μmol, 1.3 equiv.), Tris(3-hydroxypropyltriazolylmethyl)amine (2.83 mg, 6.5 μmol, 2.5 equiv.) and sodium ascorbate (1.97 mg, 9.9 μmol, 3.8 equiv.) in DMF/water (0.12 mL, 5:1). Purification by RP-HPLC afforded intermediate III.4 (9.61 mg, 70%) as yellowish solid. C₂₃₆H₃₄₄F₂N₃₀O₁₁₀. HPLC (method E): t_{R}=9.2 min. MS (ESI): found m/z=1800.6 [M+3H]³⁺, calcd. m/z=1799.7 [M+3H]³⁺.

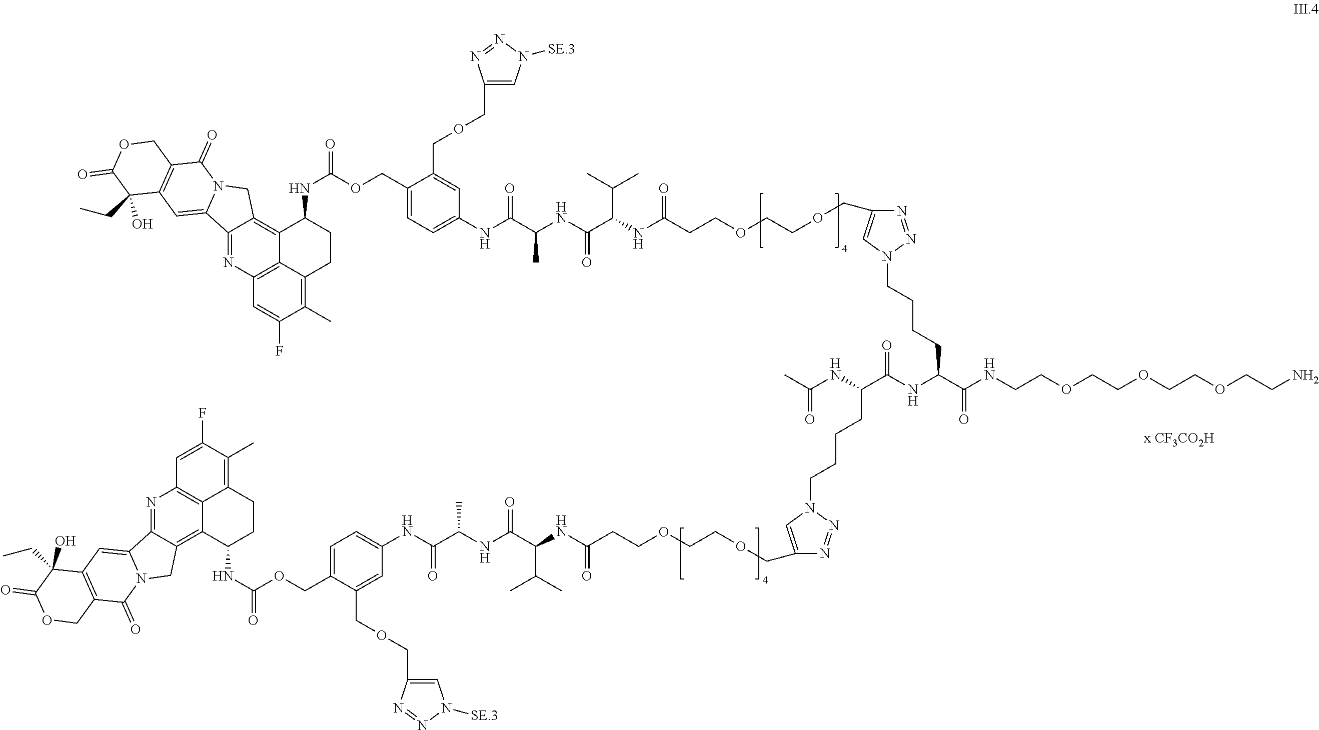

### Synthesis of IV.4

Compound IV.4 was synthesized according to GP4 using the TFA-salt of intermediate III.4 (9.61 mg, 1.74 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (1.89 mg, 3.68 μmol, 2.1 equiv.) and DIPEA (0.59 μL, 3.48 μmol, 2 equiv.) in DMF (0.4 mL). Purification by RP-HPLC afforded compound IV.4 (6.95 mg, 69%) as yellowish solid. C₂₅₄H₃₇₀F₂N₃₂O₁₁₈. HPLC (method J): t_{R}=12.9 min. MS (ESI): found m/z=1455.1 [M+3H+Na]⁴⁺, calcd. m/z=1455.7 [M+3H+Na]⁴⁺.

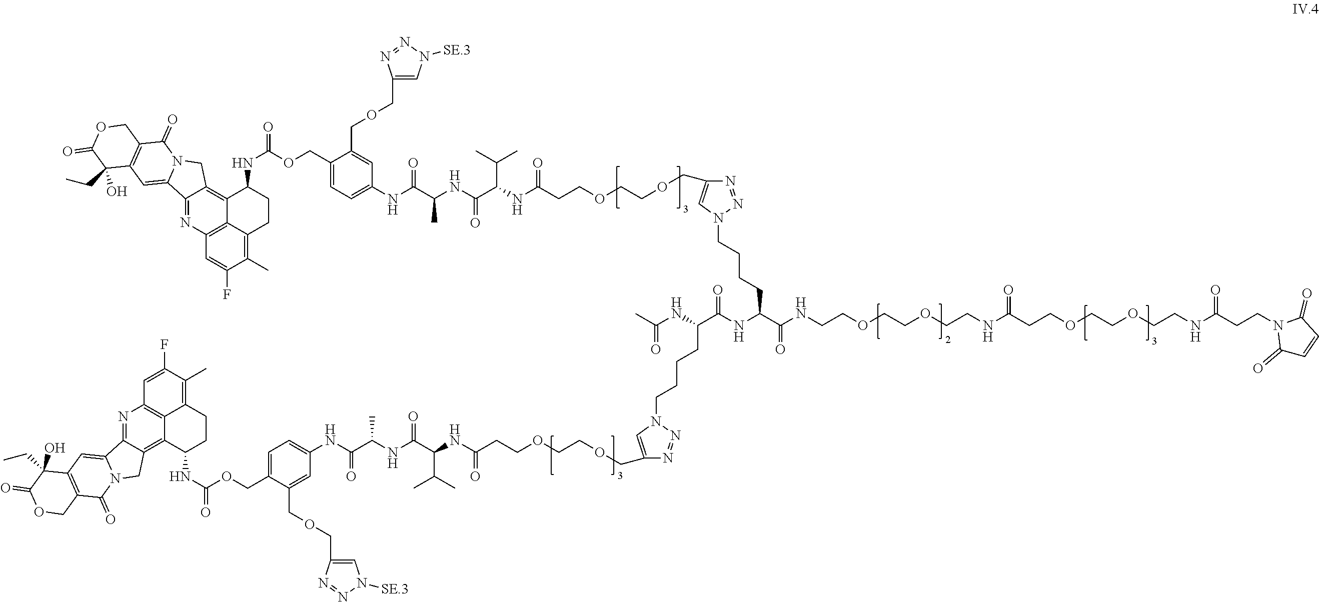

### Example 7—Synthesis of Compound IV.5

Compound IV.5 was synthesized as described for compound IV.4 but using intermediate 4.2. Compound IV.5 (25.55 mg, 68%) was obtained as yellowish solid. C₂₆₂H₃₈₆F₂N₃₂O₁₂₂. HPLC (method J): t_{R}=13.1 min. MS (ESI): found m/z=1494.3 [M+4H]⁴⁺, calcd. m/z=1493.6 [M+xH]^{x+}

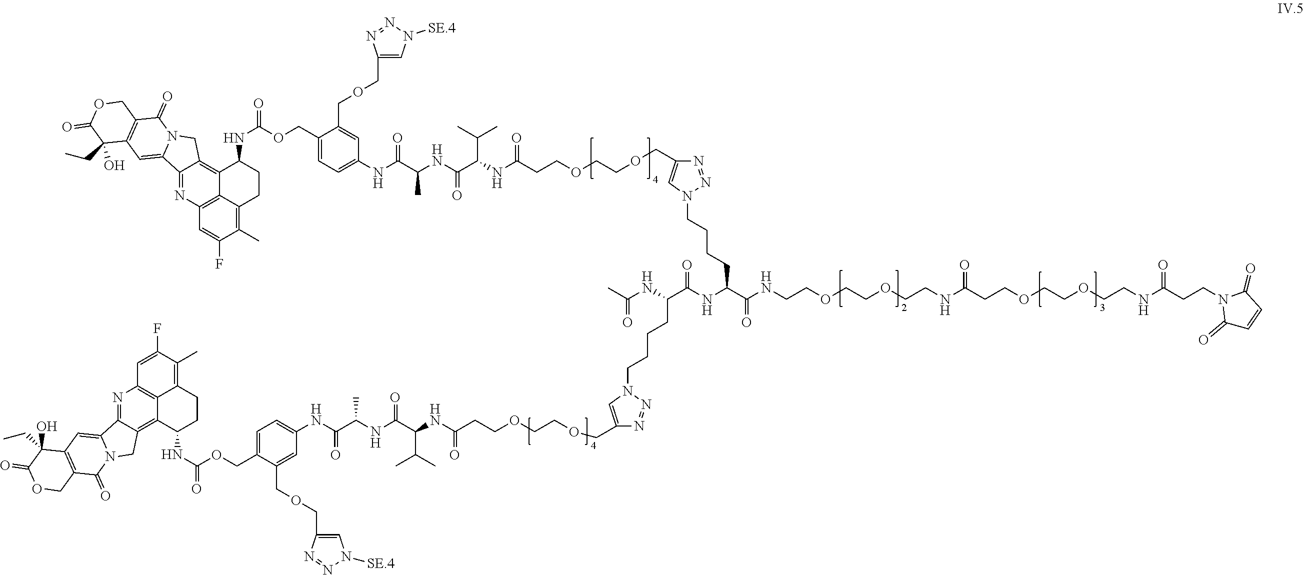

### Example 8—Synthesis of Compound IV.6

Compound IV.6 was synthesized as described for compound IV.4 but using intermediate 4.3. Compound IV.6 (6.70 mg, 83%) was obtained as yellowish solid. C₂₇₈H₄₁₈F₂N₃₂O₁₃₀. HPLC (method J): t_{R}=13.3 min. MS (ESI): found m/z=1582.3 [M+4H]⁴⁺, calcd. m/z=1581.7 [M+4H]⁴⁺.

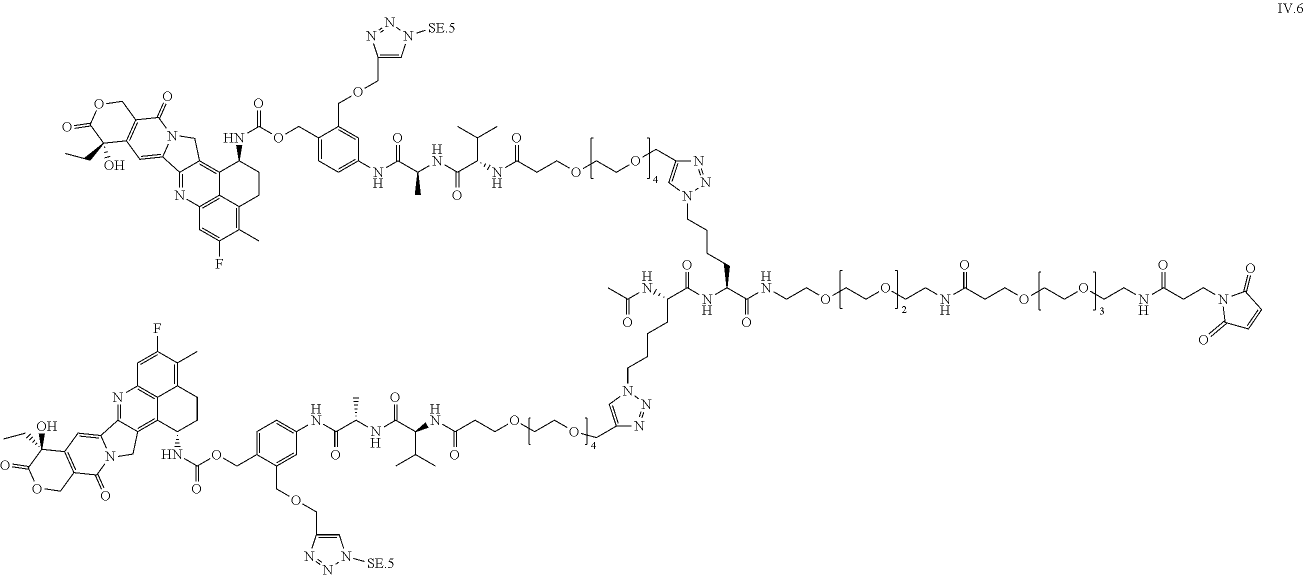

### Example 9—Synthesis of Compound IV.7

Compound IV.7 was synthesized as described for compound IV.4 but using intermediate 4.4. Compound IV.7 (10.41 mg, 51%) was obtained as yellowish solid. C₃₄₂H₅₄₆F₂N₃₂O₁₆₂. HPLC (method J): t_{R}=14.0 min. MS (ESI): found m/z=1934.3 [M+4H]⁴⁺, calcd. m/z=1933.9 [M+4H]⁴⁺.

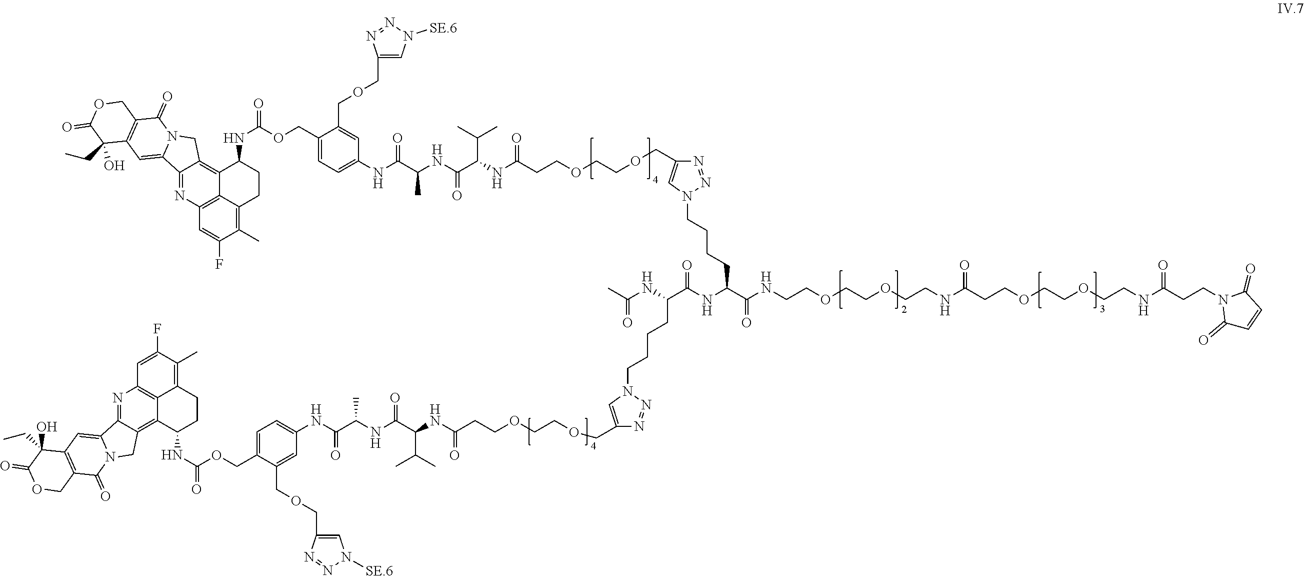

### Example 10—Synthesis of Compound IV.8

Compound IV.8 was synthesized as described for compound IV.4 but using intermediates 3.2 and 4.2. Compound IV.8 (19.3 mg, 35%) was obtained as yellowish solid. C₂₆₆H₃₉₂F₂N₃₄O₁₂₂. HPLC (method J): t_{R}=12.9 min. MS (ESI): found m/z=1514.6 [M+4H]⁴⁺, calcd. m/z=1514.1 [M+4H]⁴⁺.

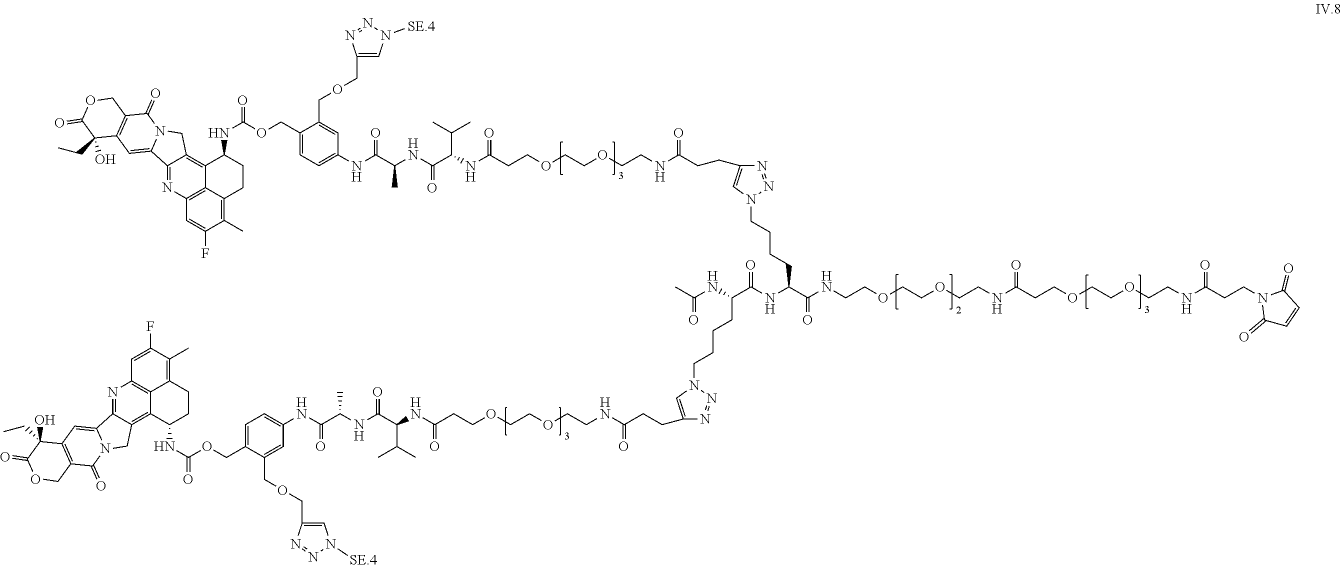

### Example 11—Synthesis of Compound IV.9

Compound IV.9 was synthesized as described for compound IV.4 but using intermediates 3.2 and 4.3. Compound IV.9 (8.87 mg, 49%) was obtained as yellowish solid. C₂₈₂H₄₂₄F₂N₃₄O₁₃₀. HPLC (method J): t_{R}=13.2 min. MS (ESI): found m/z=1602.7 [M+4H]⁴⁺, calcd. m/z=1602.2 [M+4H]⁴⁺.

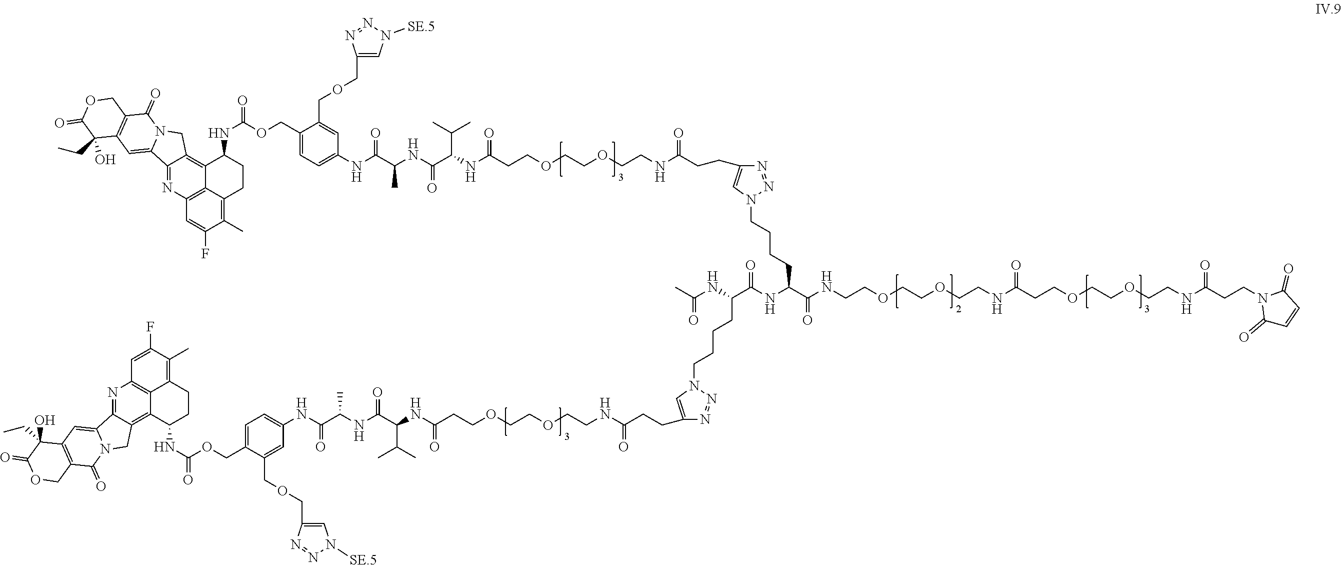

### Example 12—Synthesis of Compound IV.10

Compound IV.10 was synthesized as described for compound IV.4 but using intermediates 3.2 and 4.4. Compound IV.10 (12.26 mg, 49%) was obtained as yellowish solid. C₃₄₆H₅₂F₂N₃₄O₁₆₂. HPLC (method): t_{R}=13.9 min. MS (ESI): found m/z=1304.3 [M+6H]⁶⁺, calcd. m/z=1303.3 [M+6H]⁶⁺.

### Example 13—Synthesis of Compound IV.11

Compound IV.11 was synthesized as described for compound IV.4 but using intermediates 3.2 and 4.5. Compound IV.11 (5.69 mg, 56%) was obtained as yellowish solid. C₂₇₀H₃₉₆F₂N₃₄O₁₂₈. HPLC (method J): t_{R}=12.6 min. MS (ESI): found m/z=1551.8 [M+4H]⁴⁺, calcd. m/z=1551.1 [M+4H]⁴⁺.

### Example 14—Synthesis of Compound IV.12

Compound IV.12 was synthesized as described for compound IV.4 but using intermediates 3.2 and 4.6. Compound IV.12 (43.17 mg, 59%) was obtained as yellowish solid. C₂₇₈H₄₁₂F₂N₃₄O₁₃₂. HPLC (method J): t_{R}=12.4 min. MS (ESI): found m/z=1596.8 [M+4H]⁴⁺, calcd. m/z=1595.67 [M+4H]⁴⁺.

### Example 15—Synthesis of Compound IV. 13

Compound IV.13 was synthesized as described for compound IV.4 but using intermediates 3.2 and 4.7. Compound IV.13 (29.51 mg, 59%) was obtained as yellowish solid. C₂₉₄H₄₁F₂N₃₄O₁₄₀. HPLC (method): t_{R}=12.7 min. MS (ESI): found m/z=1684.5 [M+4H]⁴⁺, calcd. m/z=1683.2 [M+4H]⁴⁺.

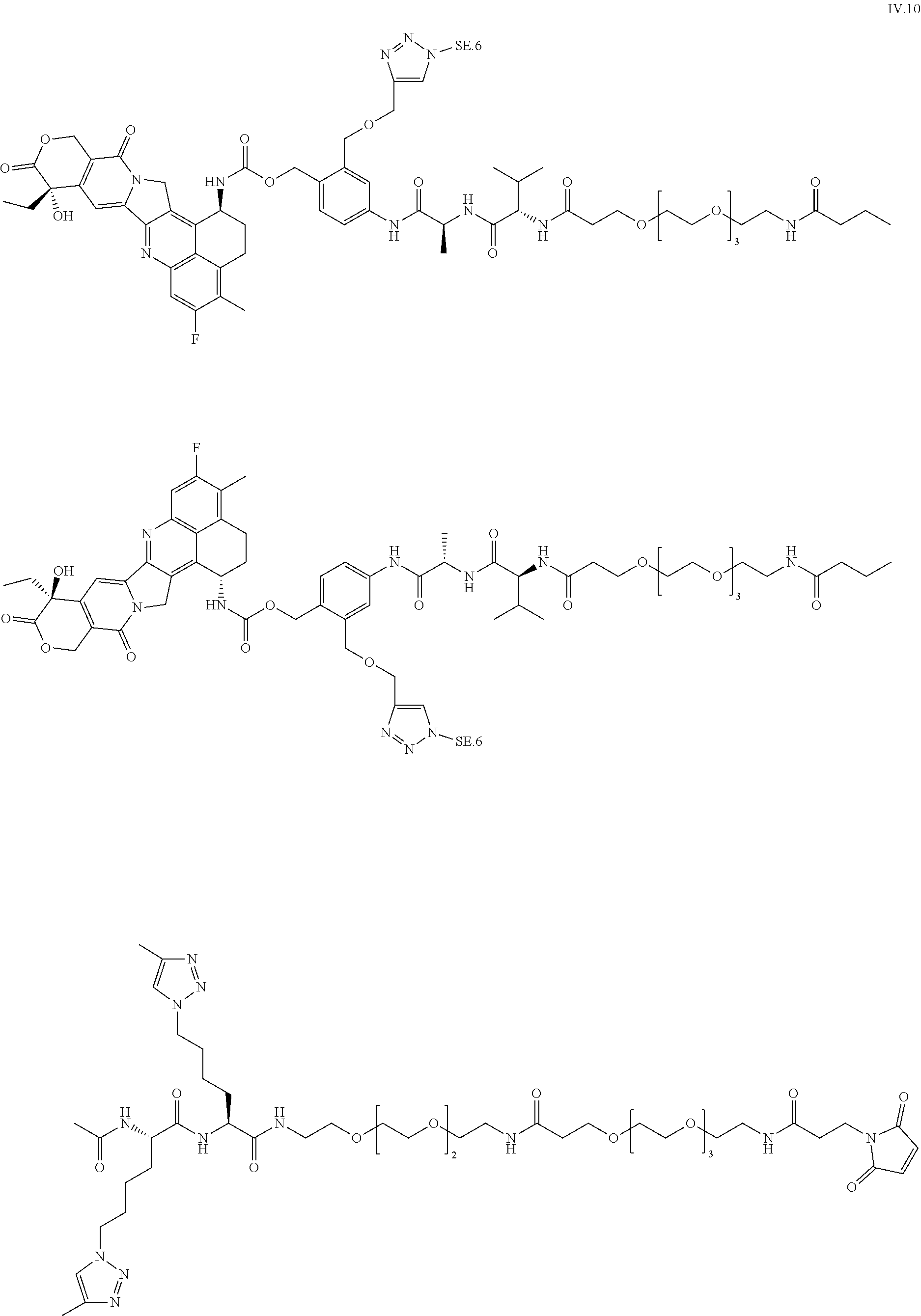

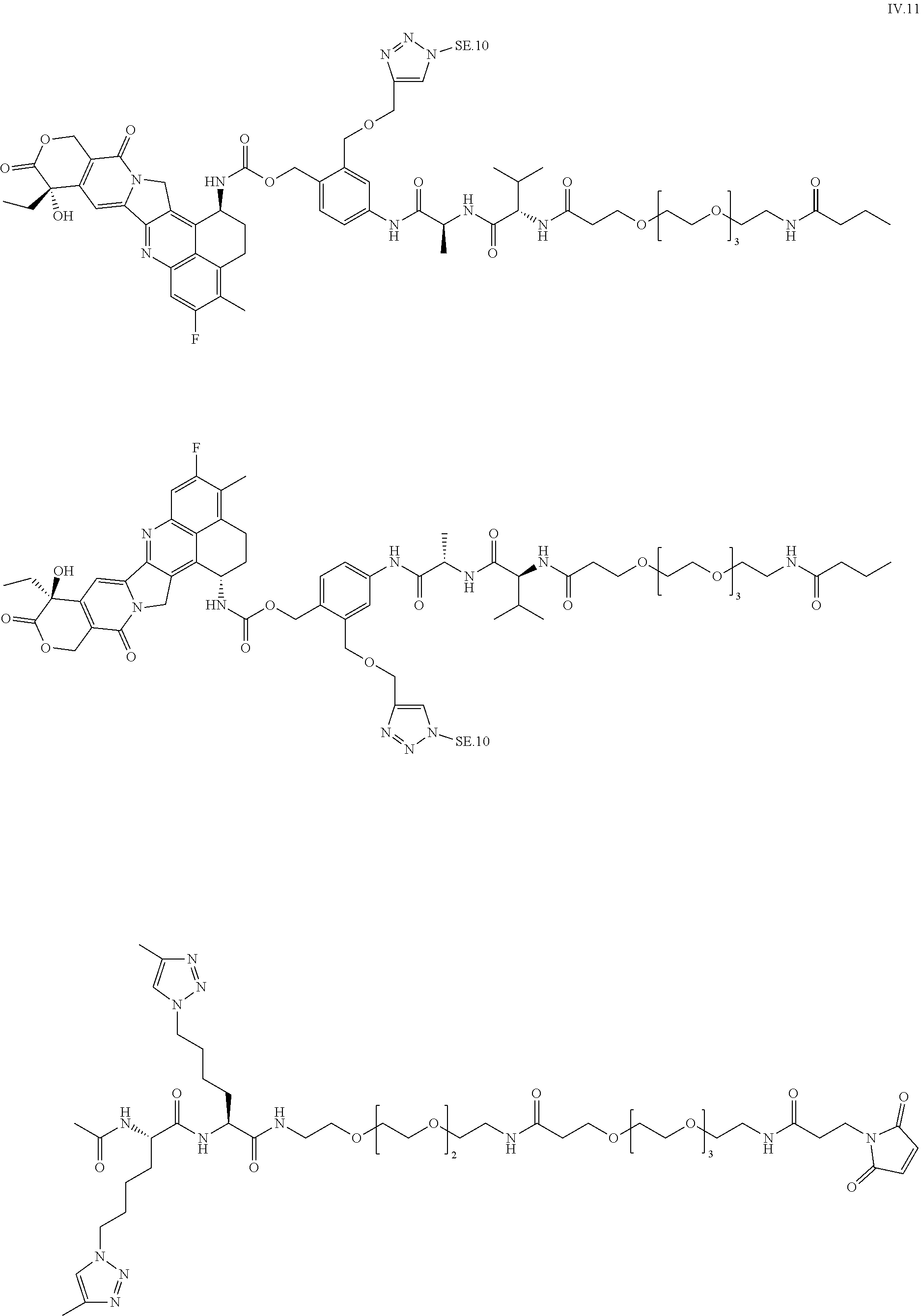

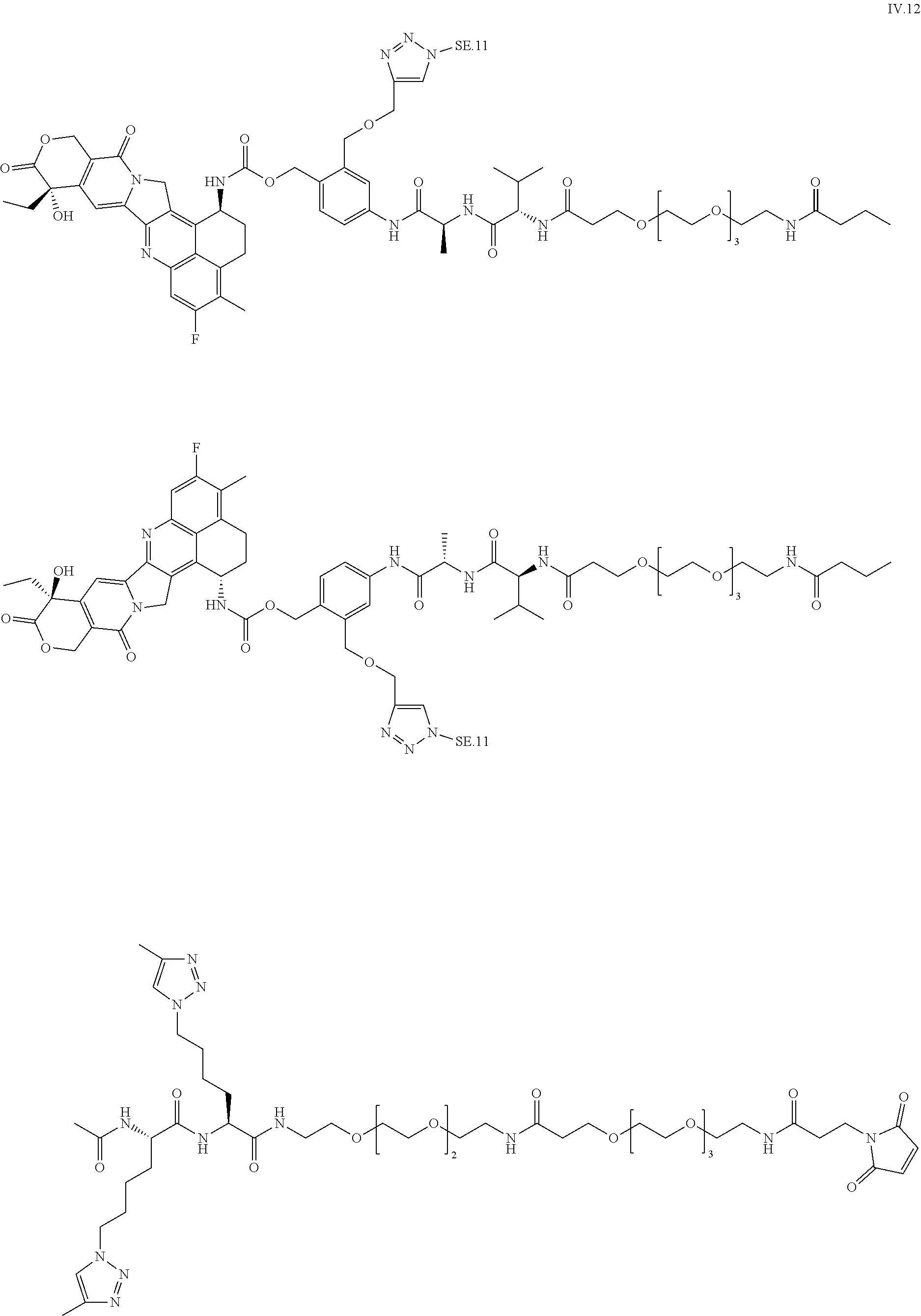

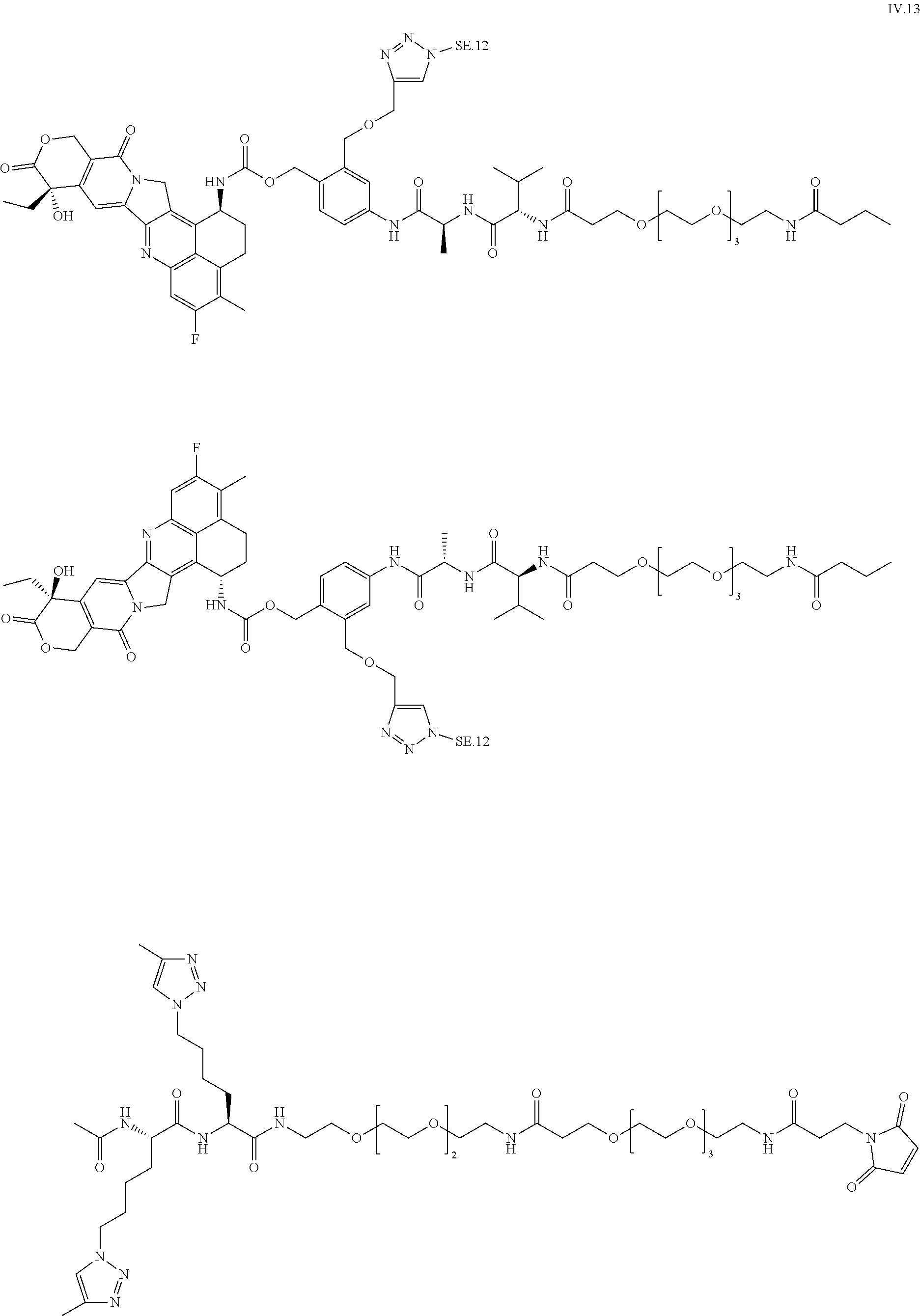

### Example 16—Synthesis of Compound IV.14

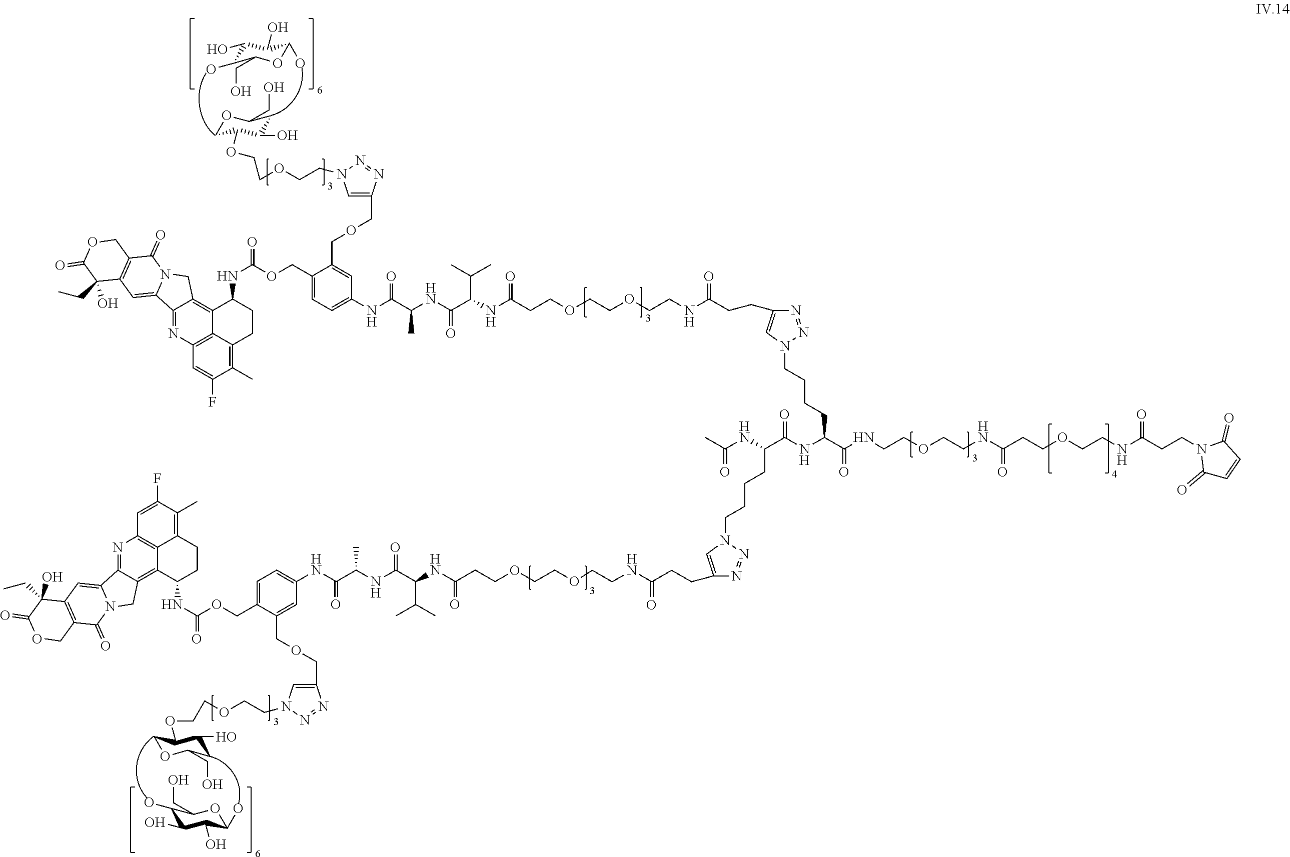

Compound IV.14 was synthesized as described for compound IV.4 but using intermediates 3.2 and 4.8. Compound IV.14 (61.21 mg, 81%) was obtained as yellowish solid. C₂₆₀H₃₈₂F₂N₃₂O₁₂₀. HPLC (method J): t_{R}=12.7 min. MS (ESI): found m/z=1479.8 [M+4H]⁴⁺, calcd. m/z=1478.6 [M+4H]⁴⁺.

### Example 17—Synthesis of Compound IV.15

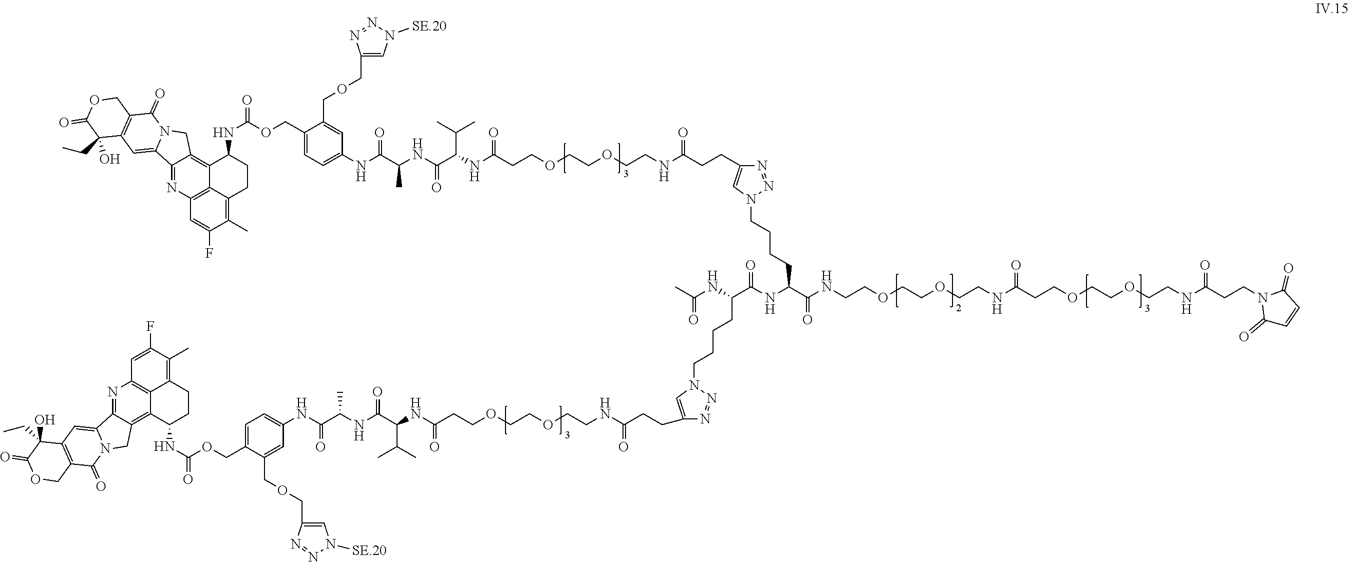

Compound IV.15 was synthesized as described for compound IV.4 but using intermediates 3.2 and 4.9. Compound IV.15 (29.68 mg, 68%) was obtained as yellowish solid. C₂₇₂H₄₀₂F₂N₃₂O₁₃₀. HPLC (method J): t_{R}=12.5 min. MS (ESI): found m/z=1560.1 [M+4H]⁴⁺, calcd. m/z=1559.7 [M+4H]⁴⁺.

### Example 18—Synthesis of Compound IV.16

### Synthesis of I.16

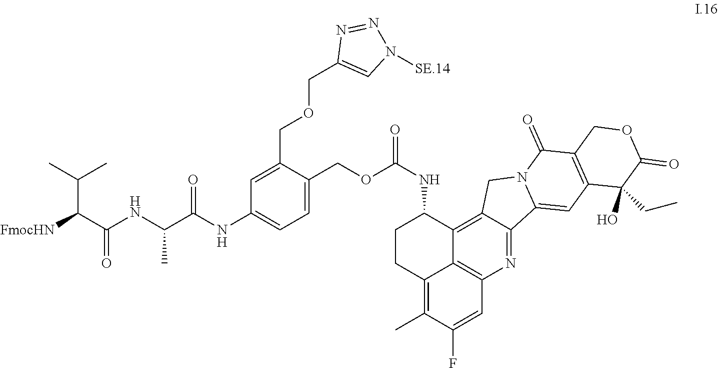

Intermediate I.16 was synthesized according to GP1 using intermediate 5.1 (30 mg, 37.0 μmol, 1 equiv.), intermediate 1.3 (42.5 mg, 40.7 μmol, 1.1 equiv.), copper (II) sulfate pentahydrate (1.85 mg, 7.4 μmol, 0.2 equiv.), tris(benzyltriazolylmethyl)amine (4.91 mg, 9.25 μmol, 0.25 equiv.) and sodium ascorbate (3.66 mg, 18.5 μmol, 0.5 equiv.) in DMF/water (0.33 mL, 10:1). Purification by RP-HPLC afforded intermediate I.16 (51.7 mg, 74%) as yellowish solid. C₉₁H₁₁₀FN₁₉O₂₃. HPLC (method A): t_{R}=11.1 min. MS (ESI): found m/z=1855.1 [M−H], calcd. m/z=1854.8 [M−H]⁻.

### Synthesis of II.16

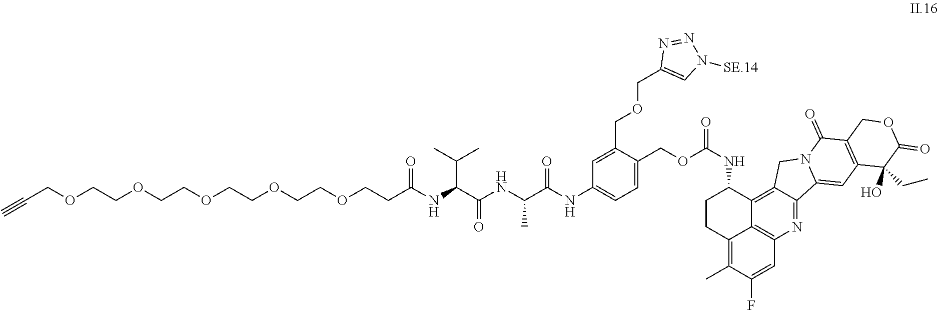

Intermediate II.16 was synthesized according to GP3 using intermediate 1.16 (51.7 mg, 27.8 μmol, 1 equiv.), diethylamine (68.7 μL, 667.0 μmol, 24 equiv.) in 2.5 mL DMF for Fmoc-deprotection and intermediate 3.1 (18.7 mg, 41.7 μmol, 1.5 equiv.), diisopropylethylamine (14.2 μL, 83.4 μmol, 3 equiv.) in DMF (3.44 mL) for the coupling reaction. Purification by RP-HPLC afforded intermediate II.16 (43.8 mg, 82%) as yellowish solid. C₉₀H₁₂₂FN₁₉O₂₇. HPLC (method A): t_{R}=9.4 min. MS (ESI): found m/z=961.6 [M+2H]²⁺, calcd. m/z=960.9 [M+2H]²⁺.

### Synthesis of III. 16

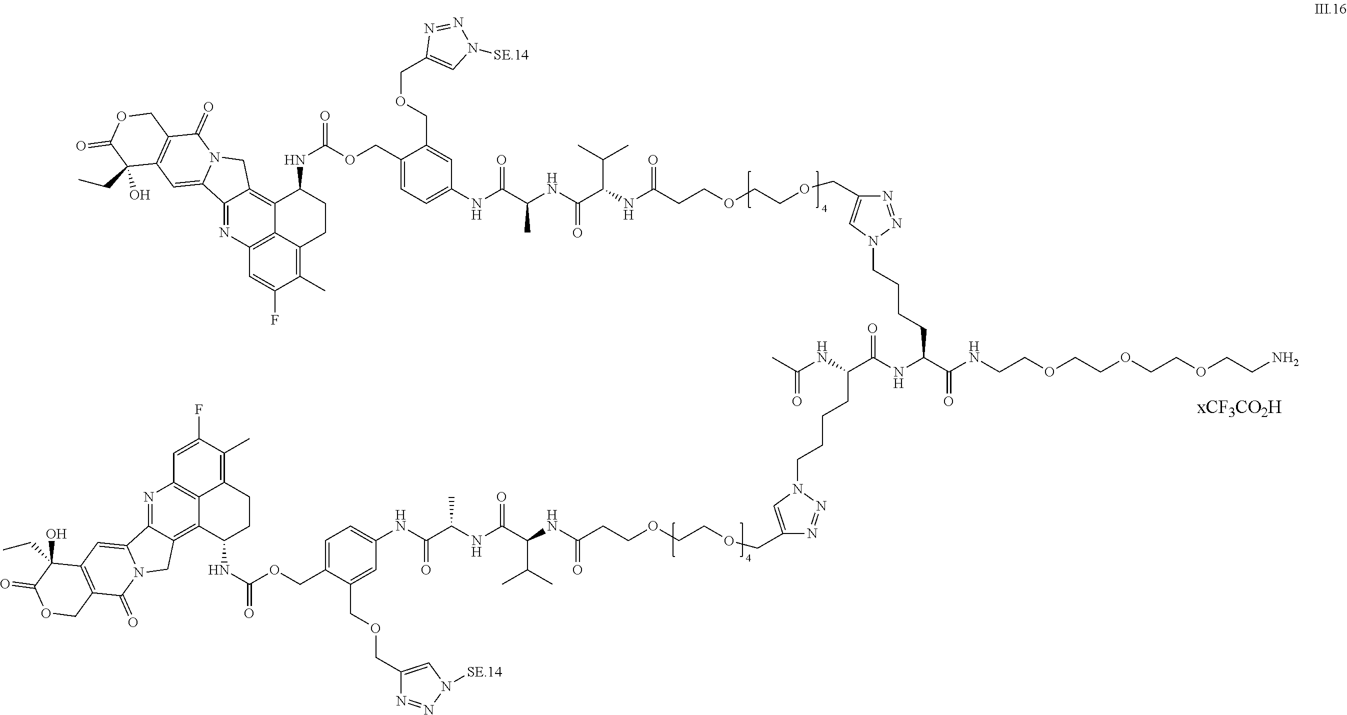

Intermediate III. 16 was synthesized according to GP2 using intermediate 2.1 (2.67 mg, 4.6 μmol, 1 equiv.), intermediate II.16 (20.4 mg, 10.6 μmol, 2.3 equiv), copper (II) sulfate pentahydrate (1.44 mg, 5.8 μmol, 1.25 equiv.), Tris(3-hydroxypropyltriazolylmethyl)amine (5.02 mg, 11.6 μmol, 2.5 equiv.) and sodium ascorbate (3.43 mg, 17.3 μmol, 3.8 equiv.) in DMF/water (0.44 mL, 10:1). Purification by RP-HPLC afforded intermediate III. 16 (9.2 mg, 46%) as yellowish solid. C₂₀₂H₂₈₆F₂N₄₈O₆₀. HPLC (method L): t_{R}=16.8 min. MS (ESI): found m/z=1462.5 [M+3H]³⁺, calcd. m/z=1461.7 [M+3H]³⁺.

### Synthesis of IV.16

Compound IV.16 was synthesized according to GP4 using the TFA-salt of intermediate III. 16 (7.4 mg, 1.26 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (0.72 mg, 1.39 μmol, 1.1 equiv.) and DIPEA (0.22 μL, 1.26 μmol, 1 equiv.) in DMF (0.3 mL). Purification by RP-HPLC afforded compound IV.16 (3.85 mg, 61%) as yellowish solid. C₂₂₀H₃₁₂F₂N₅₀O₆₈. HPLC (method J): t_{R}=14.1 min. MS (ESI): found m/z=1595.3 [M+3H]³⁺, calcd. m/z=1594.4 [M+3H]³⁺.

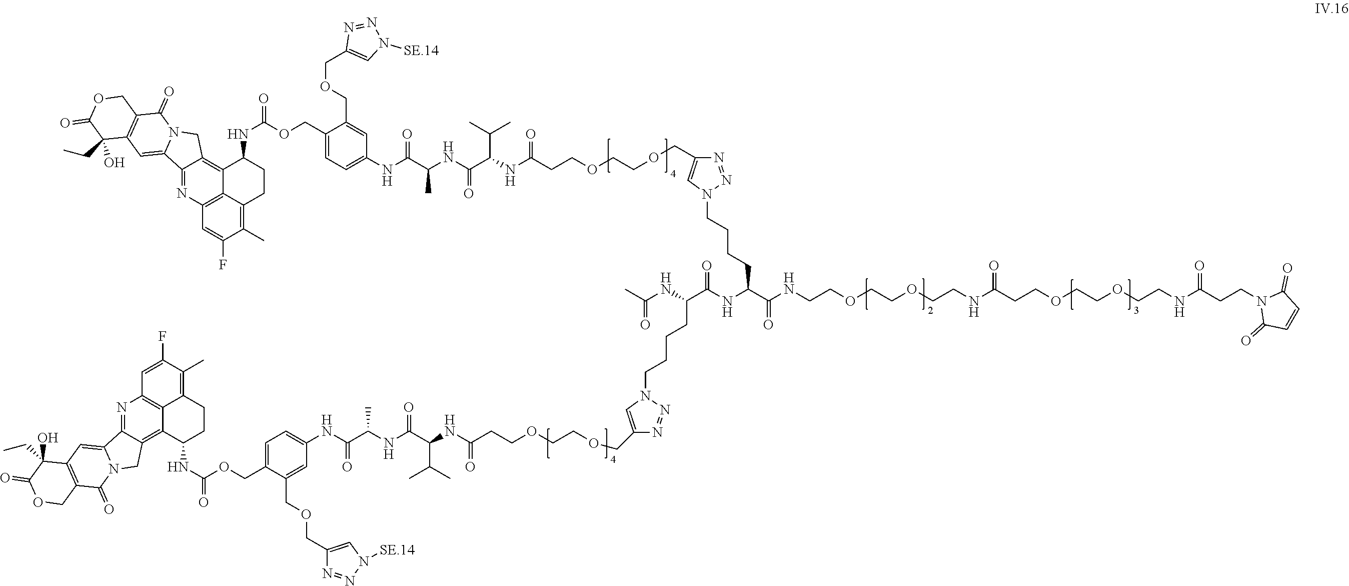

### Example 19—Synthesis of Compound IV.17

Compound IV.17 was synthesized as described for compound IV.16 but using intermediate 5.2. Compound IV.17 (23.5 mg, 75%) was obtained as yellowish solid. C₂₂₀H₃₁₄F₂N₅₂O₆₆. HPLC (method J): t_{R}=13.8 min. MS (ESI): found m/z=1594.2 [M+3H]³⁺, calcd. m/z=1593.8 [M+3H]³⁺.

### Example 20—Synthesis of Compound IV.18

Compound IV.18 was synthesized as described for compound IV.16 but using intermediates 3.2 and 5.2. Compound IV.18 (15.9 mg, 57%) was obtained as yellowish solid. C₂₂₄H₃₂₀F₂N₅₄O₆₆. HPLC (method J): t_{R}=12.9 min. MS (ESI): found m/z=1621.8 [M+3H]³⁺, calcd. m/z=1621.1 [M+3H]³⁺.

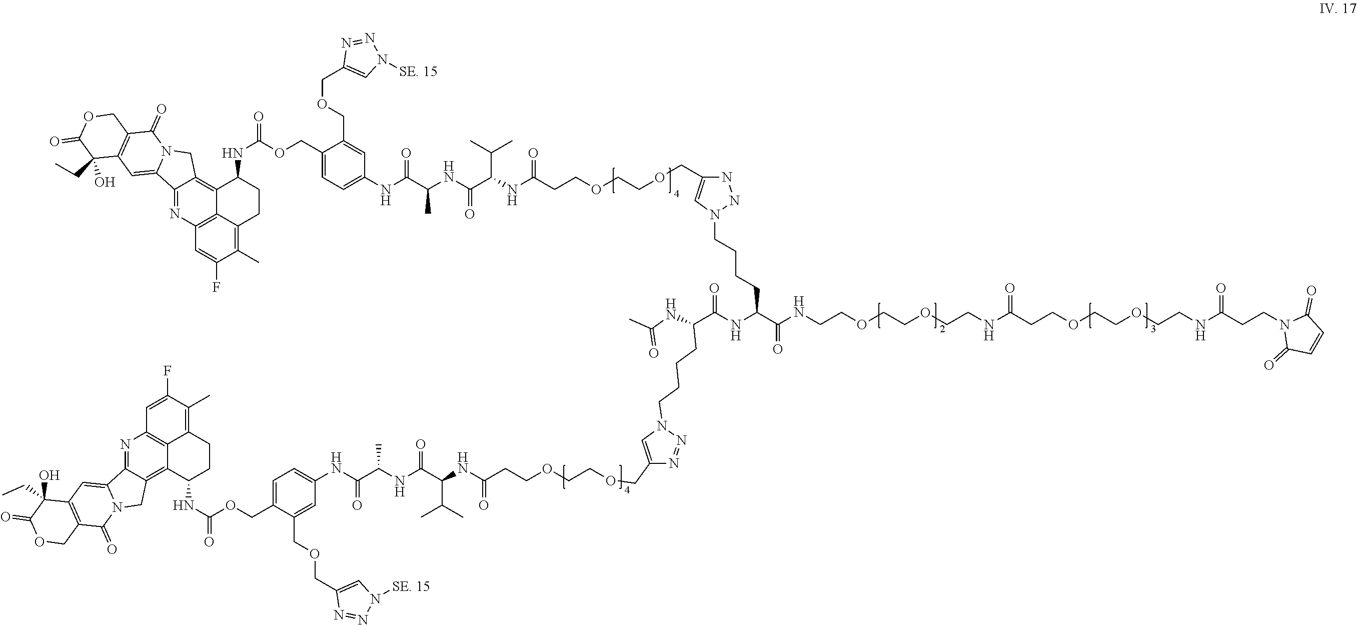

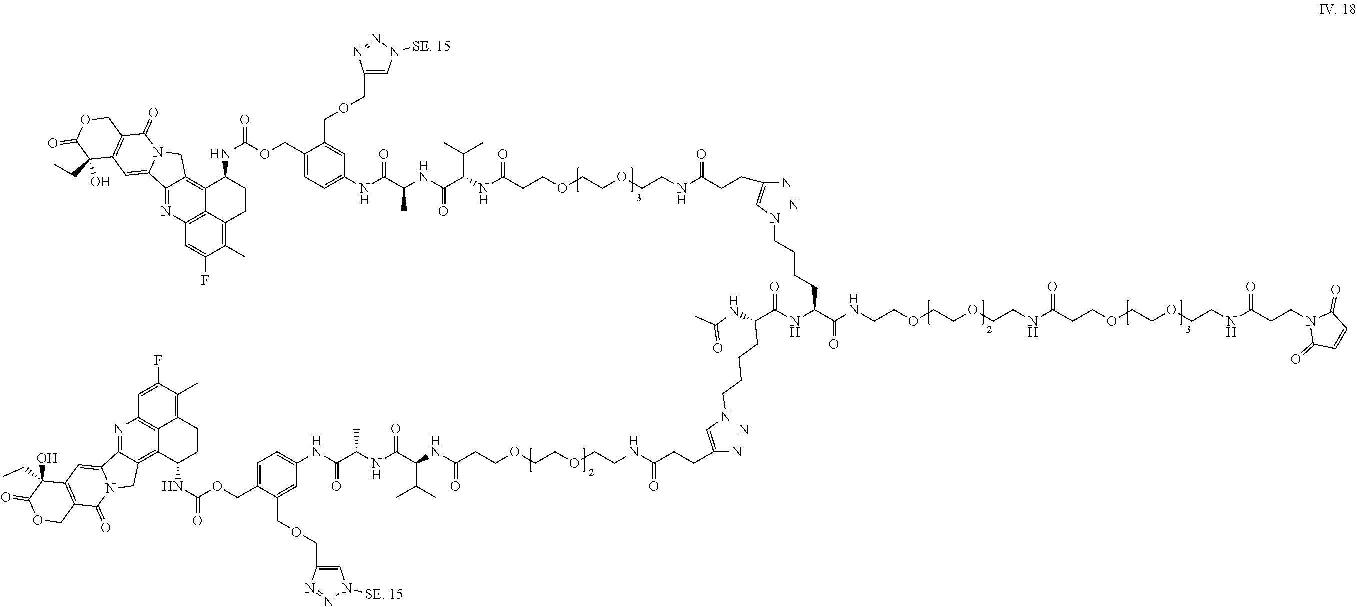

### Example 21—Synthesis of Compound IV. 19

### Synthesis of I.19

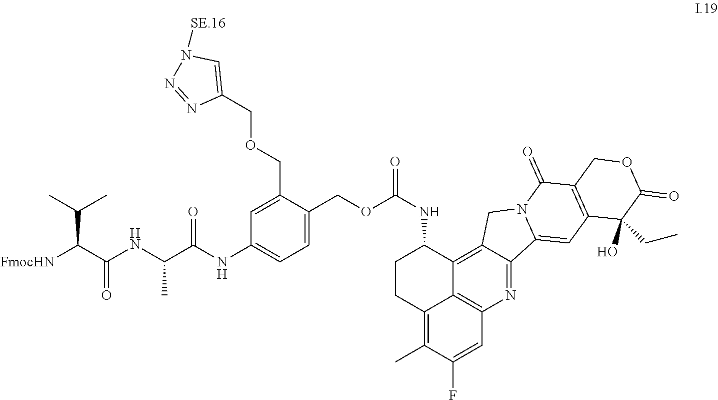

Intermediate I.19 was synthesized according to GP1 using intermediate 6.1 (64.31 mg, 86.11 μmol, 1 equiv.), intermediate 1.3 (60.0 mg, 57.4 μmol, 1.0 equiv.), copper (II) sulfate pentahydrate (14.3 mg, 57.4 μmol, 1 equiv.), THPTA (50.08 mg, 115.2 μmol, 2 equiv.) and sodium ascorbate (34.38 mg, 173.5 μmol, 3 equiv.) in DMF/water (1.72 mL, 2:1). Purification by RP-HPLC afforded intermediate 1.17 (76.18 mg, 74%) as yellowish solid. C₈₇H₁₁₁FN₁₂O₂₈. HPLC (method B): t_{R}=16.8 min. MS (ESI): found m/z=896.7 [M+2H]²⁺, calcd. m/z=896.4 [M+2H]²⁺.

### Synthesis of II.19

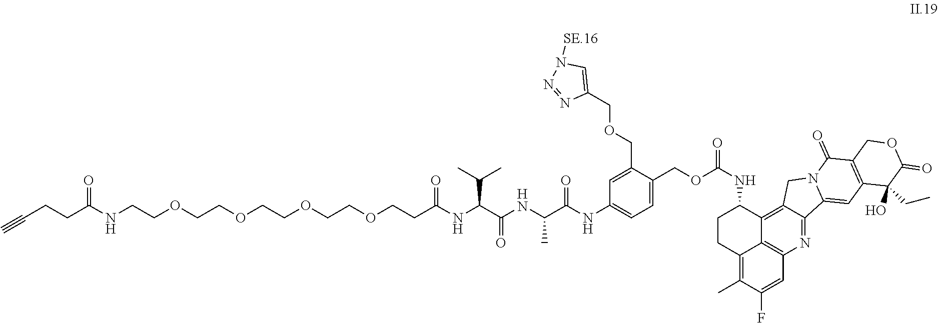

Intermediate II.19 was synthesized according to GP3 using intermediate I.19 (76.18 mg, 42.51 μmol, 1 equiv.), diethylamine (105 μL, 1020 μmol, 24 equiv.) in 5 mL DMF (abs.) for Fmoc-deprotection and intermediate 3.2 (31.28 mg, 63.8 μmol, 1.5 equiv.), diisopropylethylamine (21.7 μL, 127.5 μmol, 3 equiv.) in DMF (1 mL) for the coupling reaction. Purification by RP-HPLC afforded intermediate II.19 (38.97 mg, 48%) as yellowish solid. C₈₈H₁₂₆FN₁₃O₃₂. HPLC (method N): t_{R}=16.1 min. MS (ESI): found m/z=949.6 [M+2H]²⁺, calcd. m/z=948.9 [M+2H]²⁺.

### Synthesis of III. 19

Intermediate III. 19 was synthesized according to GP2 using intermediate 2.1 (4.96 mg, 8.6 μmol, 1 equiv.), intermediate II.19 (38.97 mg, 20.5 μmol, 2.4 equiv), copper (II) sulfate pentahydrate (2.57 mg, 10.3 μmol, 1.2 equiv.), Tris(3-hydroxypropyltriazolylmethyl)amine (8.95 mg, 20.6 μmol, 2.4 equiv.) and sodium ascorbate (6.10 mg, 30.8 μmol, 3.6 equiv.) in DMF/water (1.13 mL, 10:1). After 3 h, the same catalyst amount was added again and the solution stirred for additional 40 min at room temperature. Purification by RP-HPLC afforded intermediate III. 19 (29.03 mg, 76%) as yellowish solid. C₁₉₈H₂₉₄F₂N₃₆₀₇₀. HPLC (method O): t_{R}=12.3 min. MS (ESI): found m/z=1446.6 [M+3H]³⁺, calcd. m/z=1245.7 [M+3H]³⁺.

### Synthesis of IV.19

Compound IV.19 was synthesized according to GP4 using the TFA-salt of intermediate III. 19 (29.03 mg, 6.5 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (13.40 mg, 26.1 μmol, 4 equiv.) and DIPEA (4.43 μL, 26.1 μmol, 4 equiv.) in DMF (0.78 mL). Upon completion after 1.5 h, the product was precipitated from ice-cold MTBE acidified with TFA (6 equiv.) and purification by RP-HPLC afforded compound IV.17 (21.48 mg, 70%) as yellowish solid. C₂₁₆H₃₂₀F₂N₃₈O₇₈. HPLC (method J): t_{R}=13.3 min. MS (ESI): found m/z=1578.7 [M+3H]³⁺, calcd. m/z=1578.4 [M+3H]³⁺.

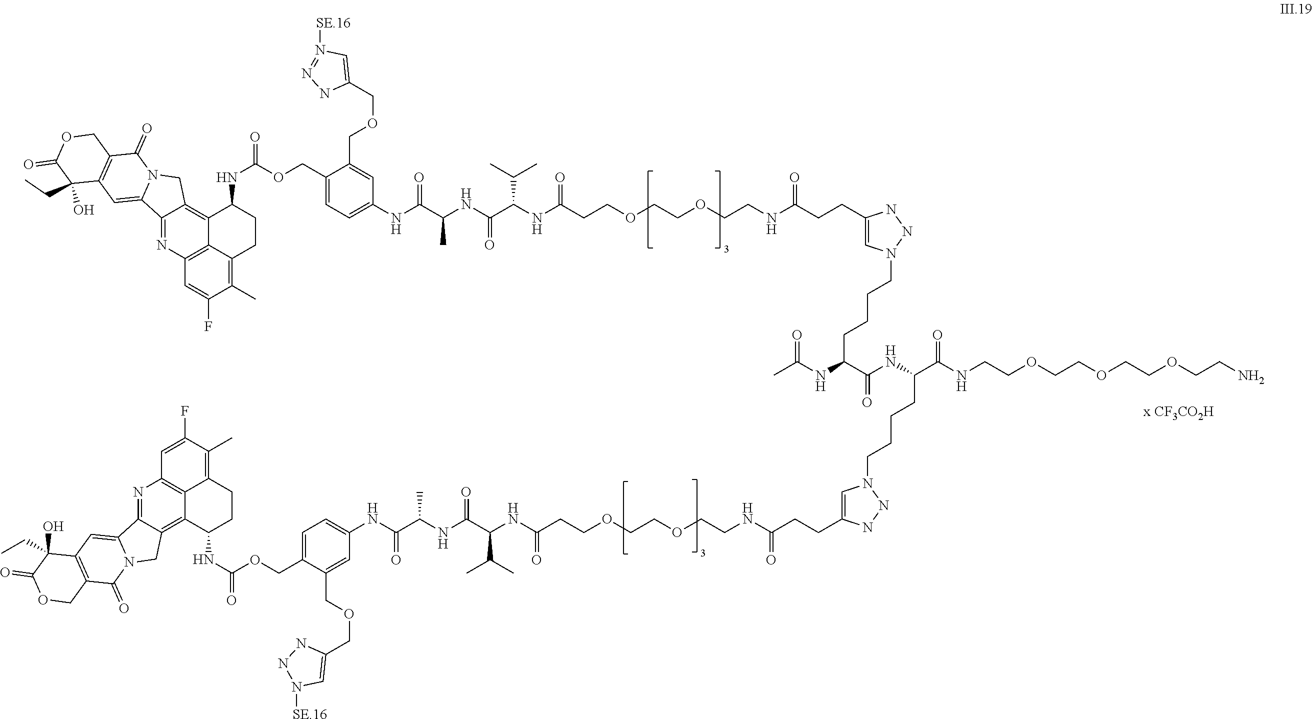

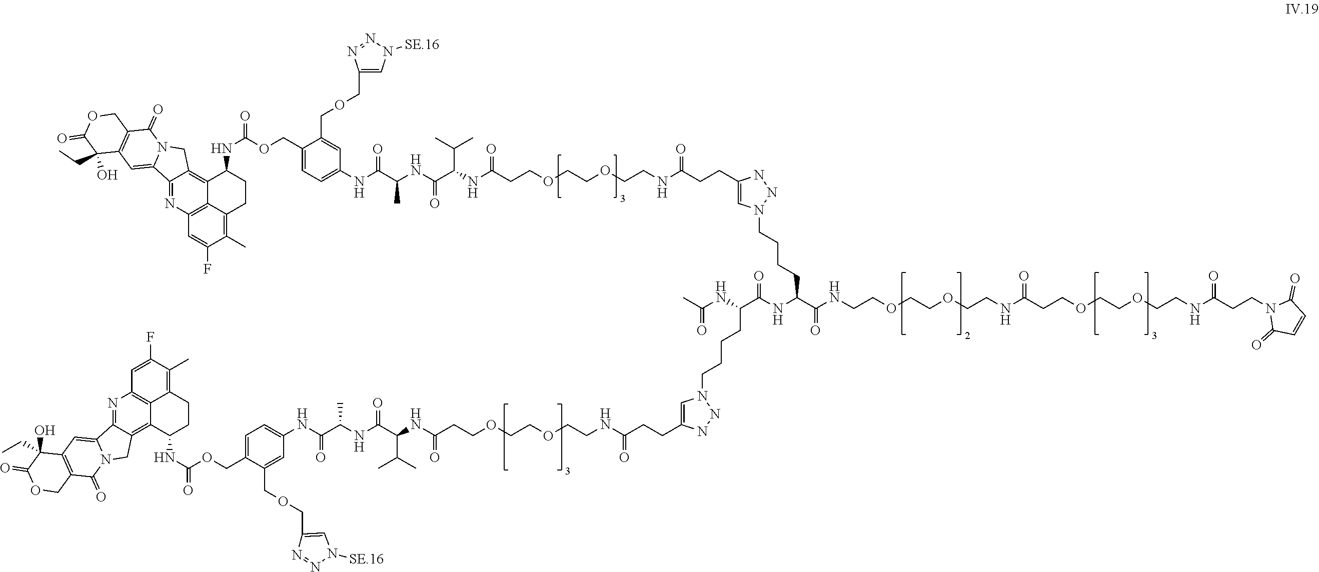

### Example 22—Synthesis of IV.20

Compound IV.20 was synthesized as described for compound IV.19 but using intermediate 6.2. Compound IV.20 (14.34 mg, 31%) was obtained as yellowish solid. C₁₉₂H₂₇₆F₂N₃₄O₆₄. HPLC (method J): t_{R}=13.5 min. MS (ESI): found m/z=1374.9 [M+3H]³⁺, calcd. m/z=1374.3 [M+3H]³⁺.

### Example 23—Synthesis of IV.21

Compound IV.21 was synthesized as described for compound IV.19 but using intermediate 6.3. Compound IV.21 (17.83 mg, 64%) was obtained as yellowish solid. C₂₁₆H₃₂₀F₂N₃₈O₇₈. HPLC (method J): t_{R}=13.1 min. MS (ESI): found m/z=1579.1 [M+3H]³⁺, calcd. m/z=1578.4 [M+3H]³⁺.

### Example 24—Synthesis of IV.22

Compound IV.22 was synthesized as described for compound IV.19 but using intermediate 6.4. Compound IV.22 (27.9 mg, 55%) was obtained as yellowish solid. C₂₃₂H₃₅₂F₂N₃₈O₈₆. HPLC (method J): t_{R}=13.4 min. MS (ESI): found m/z=1697.0 [M+3H]³⁺, calcd. m/z=1695.8 [M+3H]³⁺.

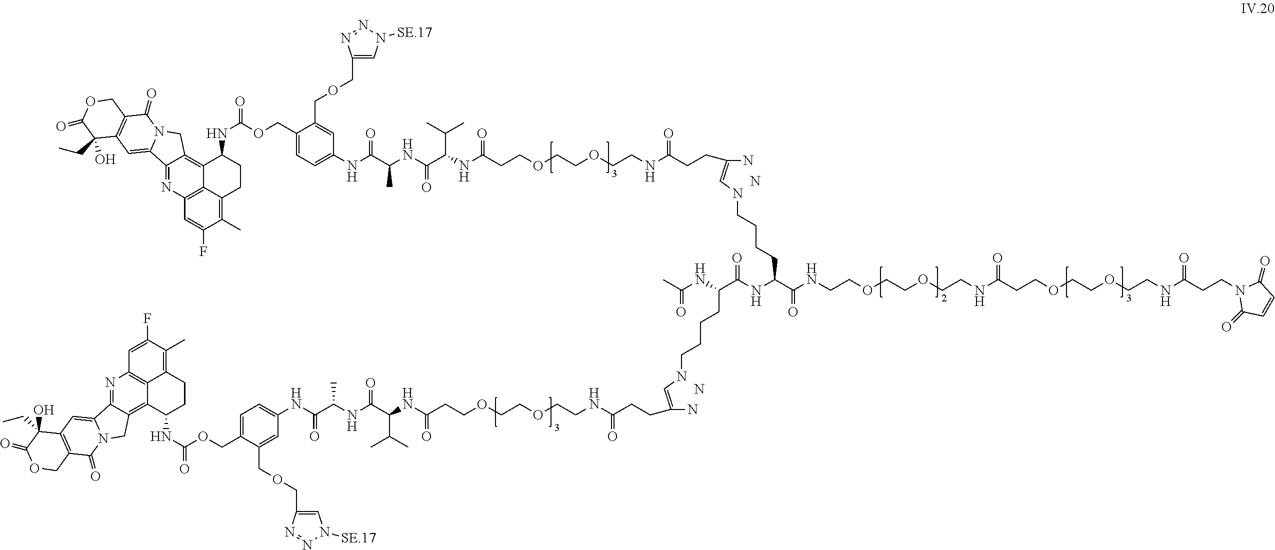

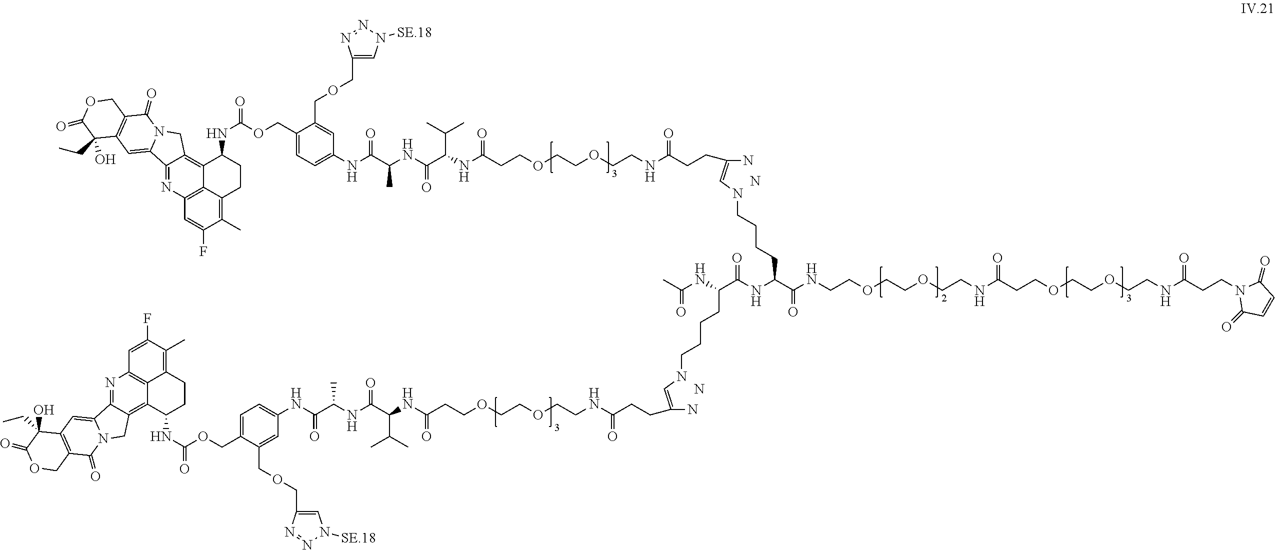

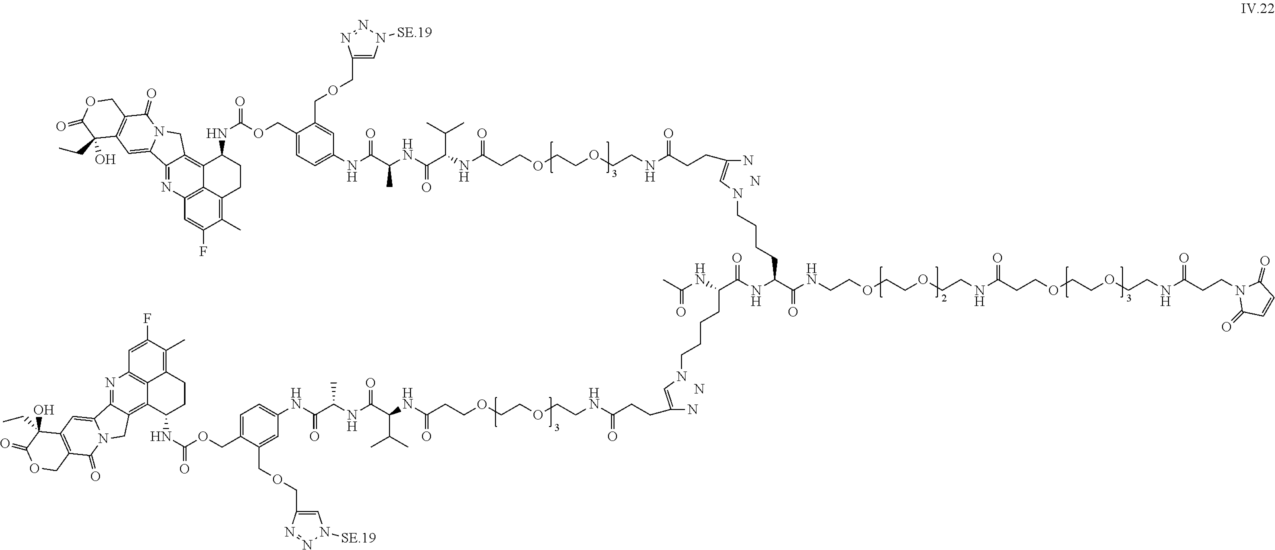

### Example 25—Synthesis of IV.23

### Synthesis of I.23a

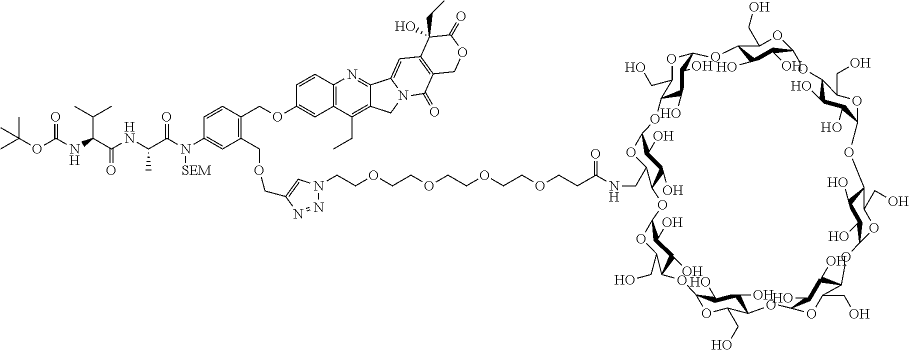 [SMILES: CCc1c2c(nc3ccc(OCc4ccc(N(COCC[Si](C)(C)C)C(=O)[C@H](C)NC(=O)[C@@H](NC(=O)OC(C)(C)C)C(C)C)cc4COCc4cn(CCOCCOCCOCCOCCC(=O)NCC5O[C@@H]6O[C@@H]7C(CO)O[C@H](O[C@@H]8C(CO)O[C@H](O[C@@H]9C(CO)O[C@H](O[C@@H]%10C(CO)O[C@@H](O[C@@H]%11C(CO)O[C@@H](O[C@@H]%12C(CO)O[C@@H](O[C@@H]%13C(CO)O[C@H](O[C@H]5[C@H](O)C6O)C(O)[C@H]%13O)C(O)[C@H]%12O)C(O)[C@H]%11O)C(O)[C@H]%10O)C(O)[C@H]9O)C(O)[C@H]8O)C(O)[C@H]7O)nn4)cc13)-c1cc3c(c(=O)n1C2)COC(=O)[C@]3(O)CC]

Compound I.23a was synthesized according to GP1 using Intermediate 4.6 (65 mg, 41.4 μmol, 1 equiv.), intermediate 4.6 (40 mg, 41.4 μmol, 1.0 equiv.), copper (II) sulfate pentahydrate (2.1 mg, 8.3 μmol, 0.2 equiv.), THPTA (4.5 mg, 10.3 μmol, 0.25 equiv.) and sodium ascorbate (4.1 mg, 20.7 μmol, 0.5 equiv.) in DMF/water (640 μL, 10:1). Purification by RP-HPLC afforded intermediate 1.23a (113 mg, quant.) HPLC (method A (380 nm)): t_{R}=10.5 min. MS (ESI): found m/z=1267.3 [M+2H]²⁺, calcd. m/z=1268.52

### Synthesis of I.23b

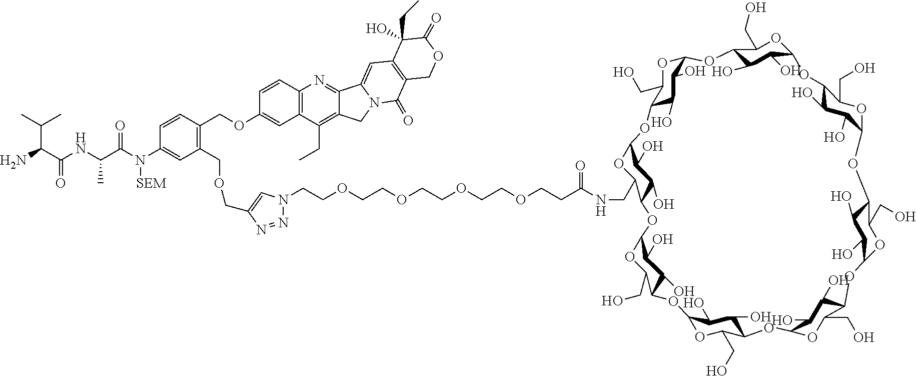 [SMILES: CCc1c2c(nc3ccc(OCc4ccc(N(COCC[Si](C)(C)C)C(=O)[C@H](C)NC(=O)[C@@H](N)C(C)C)cc4COCc4cn(CCOCCOCCOCCOCCC(=O)NCC5O[C@@H]6O[C@@H]7C(CO)O[C@H](O[C@@H]8C(CO)O[C@H](O[C@@H]9C(CO)O[C@H](O[C@@H]%10C(CO)O[C@@H](O[C@@H]%11C(CO)O[C@@H](O[C@@H]%12C(CO)O[C@@H](O[C@@H]%13C(CO)O[C@H](O[C@H]5[C@H](O)C6O)C(O)[C@H]%13O)C(O)[C@H]%12O)C(O)[C@H]%11O)C(O)[C@H]%10O)C(O)[C@H]9O)C(O)[C@H]8O)C(O)[C@H]7O)nn4)cc13)-c1cc3c(c(=O)n1C2)COC(=O)[C@]3(O)CC]

Intermediate I.23a (113.5 mg, 41 μmol, 1 equiv.) was taken up in TFA (3.14 mL, 41 mmol, 1000 equiv.). The resulting reaction mixture was rotated in a rotary evaporator at 400 mbar at room temperature. After 2 minutes the solution was concentrated to dryness. The residue was dissolved in 3 mL of H2O and adjusted to pH=10 by the dropwise addition of 1.2 mL of 3.2% aqueous ammonia at room temperature. The resulting mixture was freeze dried overnight to yield intermediate 1.23b as yellowish lyophilizate (0.047 g, 45%). HPLC (method A (380 nm)): t_{R}=7.2 min. MS (ESI): found m/z=1152.5 [M+2H]²⁺, calcd. m/z=1153.46

### Synthesis of II.23

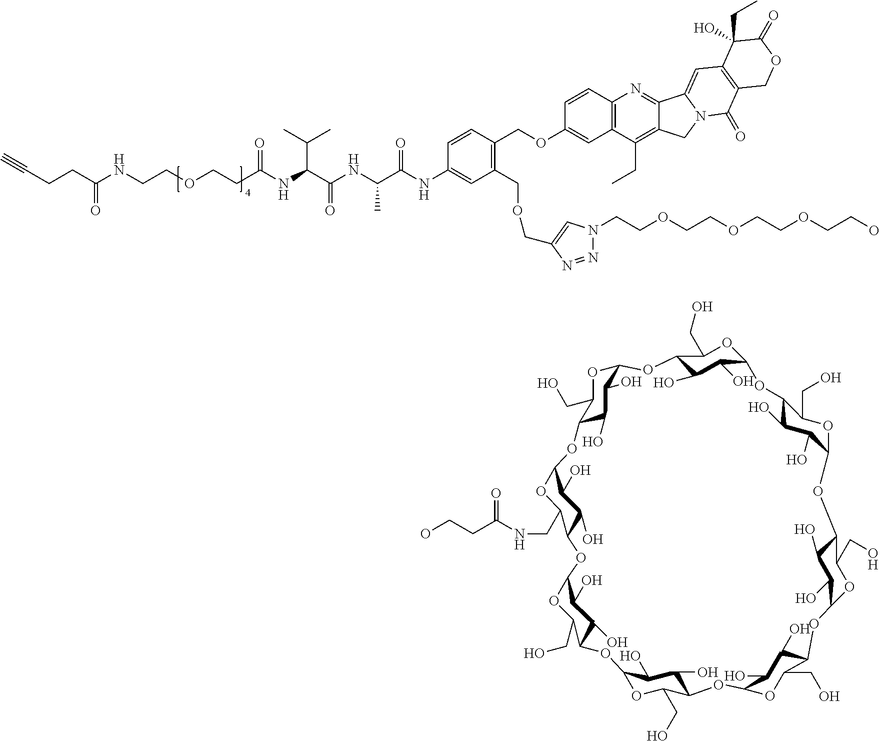

Intermediate I.23b (0.047 g, 0.02 mmol, 1 equiv.) was dissolved in 0.28 mL dry DMF. Under argon atmosphere intermediate 3.2 (0.01 g, 0.022 mmol, 1.1 equiv.) and DIPEA (0.0011 mL, 0.061 mmol, 3 equiv.) were added. The solution was stirred at room temperature and the progress followed by RP-HPLC. Upon completion (˜1 h), the reaction mixture was neutralized with TFA and purified directly without any pretreatment by prep HPLC to yield intermediate II.23 as yellowish lyophilizate (0.041 mg, 78%). HPLC (method N at 380 nm)): t_{R}=13.5 min. MS (ESI): found m/z=1316.2 [M+2H]²⁺, calcd. m/z=1317.04

### Synthesis of III.23

Intermediate III.23 was synthesized according to GP2 using intermediate 2.1 (4.6 mg, 7.9 μmol, 1 equiv.), intermediate II.23 (45.5 mg, 17.3 μmol, 2.2 equiv), copper (II) sulfate pentahydrate (4.92 mg, 19.7 μmol, 2.5 equiv.), Tris(3-hydroxypropyltriazolylmethyl)amine (17.1 mg, 39.3 μmol, 5 equiv.) and sodium ascorbate (11.7 mg, 59 μmol, 7.5 equiv.) in DMF/water (385.4 μL, 10:1). After 2 h the reaction was complete, and the reaction mixture was purified directly without any pretreatment by prep. HPLC to yield intermediate III.23 as yellowish lyophilizate (21.9 mg, 48%). C₂₅₄H₃₈₂N₃₀O. HPLC (method O): t_{R}=12.4 min. MS (ESI): found m/z=1936.2 [M+3H]³⁺, calcd. m/z=1936.16 [M+3H]³⁺.

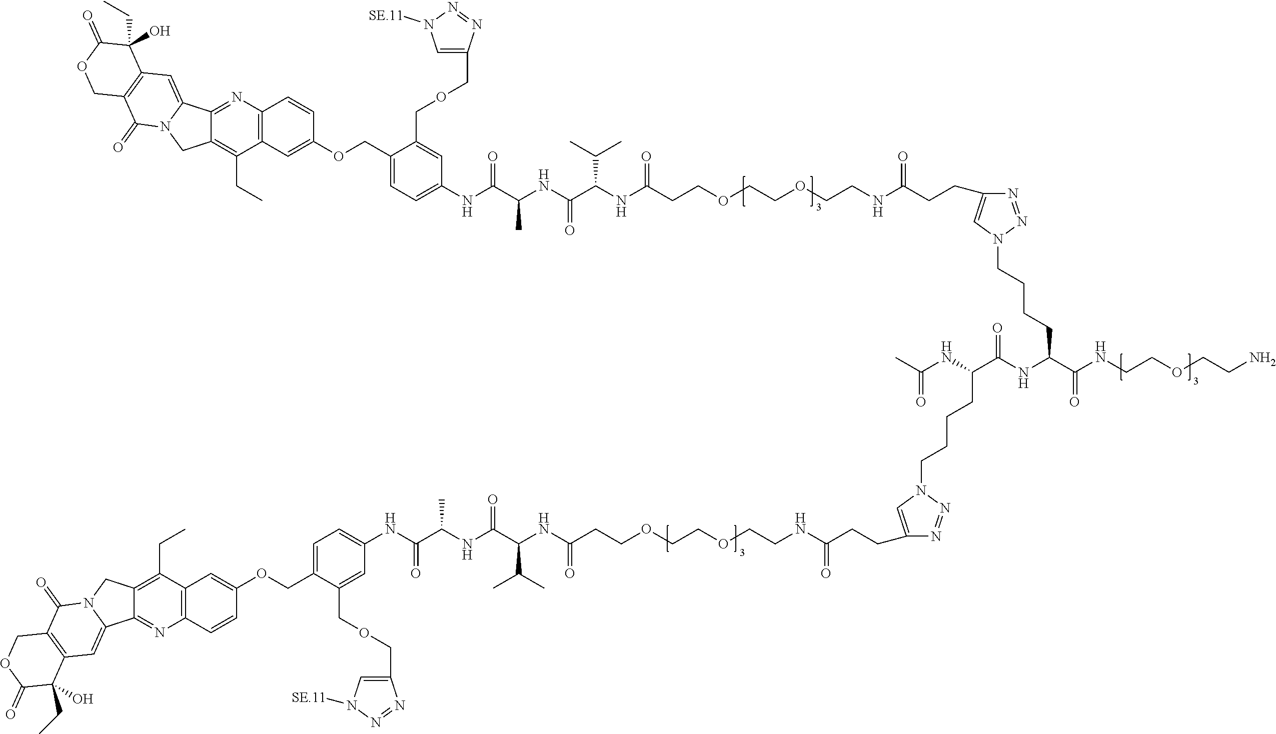

### Synthesis of IV.23

Compound IV.23 was synthesized as described for compound IV.16. Compound IV.23 (10.3 mg, 44%) was obtained as yellowish solid. C₂₇₂H₄₀₈N₃₂O₁₃₀. HPLC (method J): t_{R}=12.3 min. MS (ESI): found m/z=15551.7 [M+4H]⁴⁺, calcd. m/z=1551.41 [M+4H]⁴⁺.

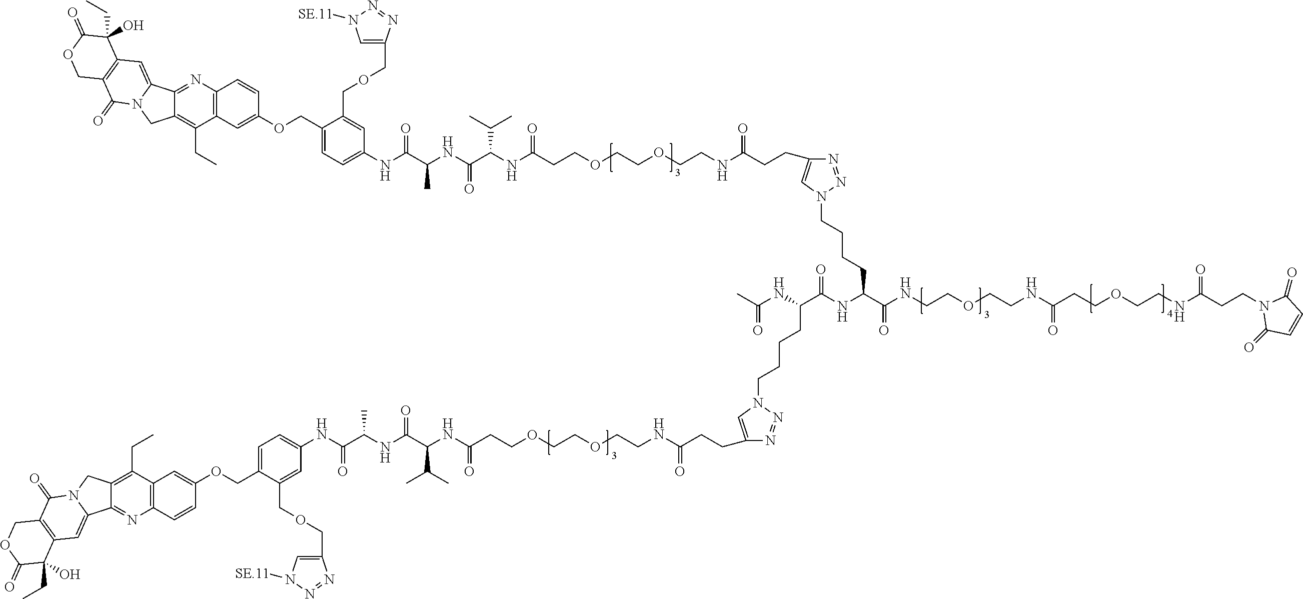

### Example 26—Synthesis of IV.24

### Synthesis of I.24

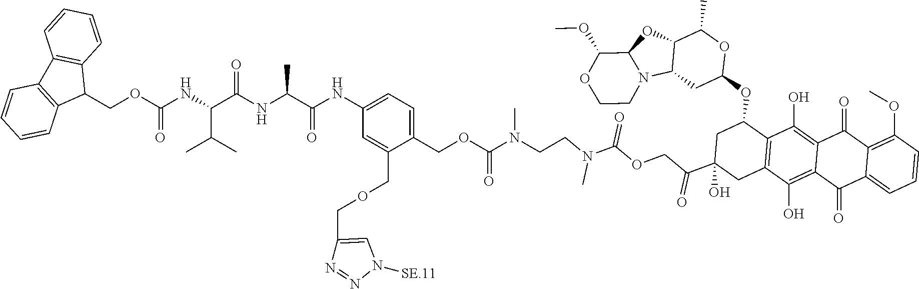

To a stirred solution of Intermediate 4.6 (31.5 mg, 20.1 μmol, 1 equiv.) and intermediate 1.16 (28.8 mg. 21.1 μmol, 1.05 equiv.) in 286 μL DMF was added 286 μL DMF containing TBTA (2.66 mg, 5.02 μmol, 0.25 equiv.) and 57.2 μL water containing, copper (II) sulfate pentahydrate (1 mg, 4.02 μmol, 0.2 equiv.) and Na-ascorbate (1.98 mg, 10 μmol, 0.5 equiv.). The reaction mixture was stirred at room temperature and the progress followed by RP-UHPLC. After 15 min the reaction mixture was added to 10 mL ice cold acetone. The resulting precipitate was isolated by centrifugation, resuspended in 6 mL cold acetone and centrifuged again. The crude product (54.3 mg, 92%) was used without further purification. UHPLC (method Q): t_{R}=2.7 min. MS (ESI): found m/z=1467.0 [M+2H]²⁺, calcd. m/z=1468.06

### Synthesis of II.24

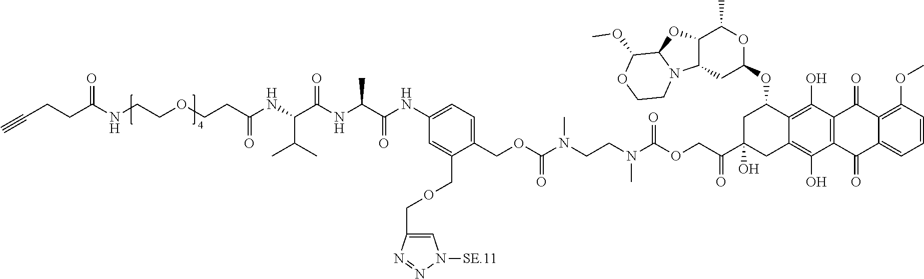

Intermediate II.24 was synthesized according to GP3 using intermediate 1.24 (54.3 mg, 18.5 μmol, 1 equiv.), 45.8 μL diethylamine (444.6 μmol, 24 equiv.) in 272 μL DMF for Fmoc-deprotection and intermediate 3.2 (10 mg, 20.4 μmol, 1.1 equiv.), diisopropylethylamine (7.57 μL, 55.5 μmol, 3 equiv.) in DMF (270 μL) for the coupling reaction. Purification by RP-HPLC afforded intermediate II.24 (17.2 mg, 31%) as red solid. C₁₃₂H₁₉₅N₁₁O₆₉. HPLC (method R): t_{R}=2.9 min. MS (ESI): found m/z=1520.61 [M+2H]²⁺, calcd. m/z=1520.0

### Synthesis of III.24

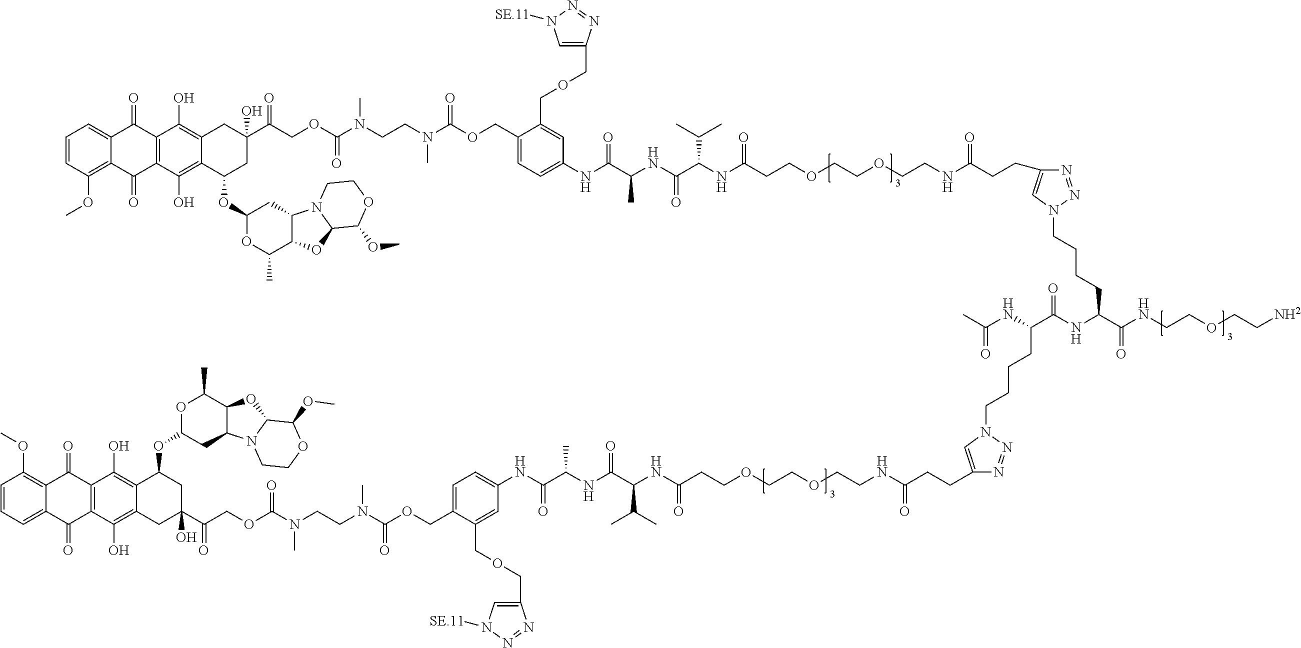

Intermediate III.24 was synthesized according to GP2 using intermediate 2.1 (1.98 mg, 3.14 μmol, 1 equiv.), intermediate II.24 (21 mg, 6.91 μmol, 2.2 equiv.), copper (II) sulfate pentahydrate (1.96 mg, 7.85 μmol, 2.5 equiv.), Tris(3-hydroxypropyltriazolylmethyl)amine (6.82 mg, 15.7 μmol, 5 equiv.) and sodium ascorbate (4.68 mg, 23.6 μmol, 7.5 equiv.) in DMF/water (165 μL, 10:1). After 30 min the reaction was complete, and the reaction mixture was purified directly without any pretreatment by prep. HPLC to yield intermediate III.24 as red lyophilizate (6.1 mg, 33%). C₂₈₆H₄₃₂N₃₂O₁₄. UHPLC (method S): t_{R}=3.3 min. MS (ESI): found m/z=1655.6 [M+4H]⁴⁺, calcd. m/z=1655.69

### Synthesis of IV.24

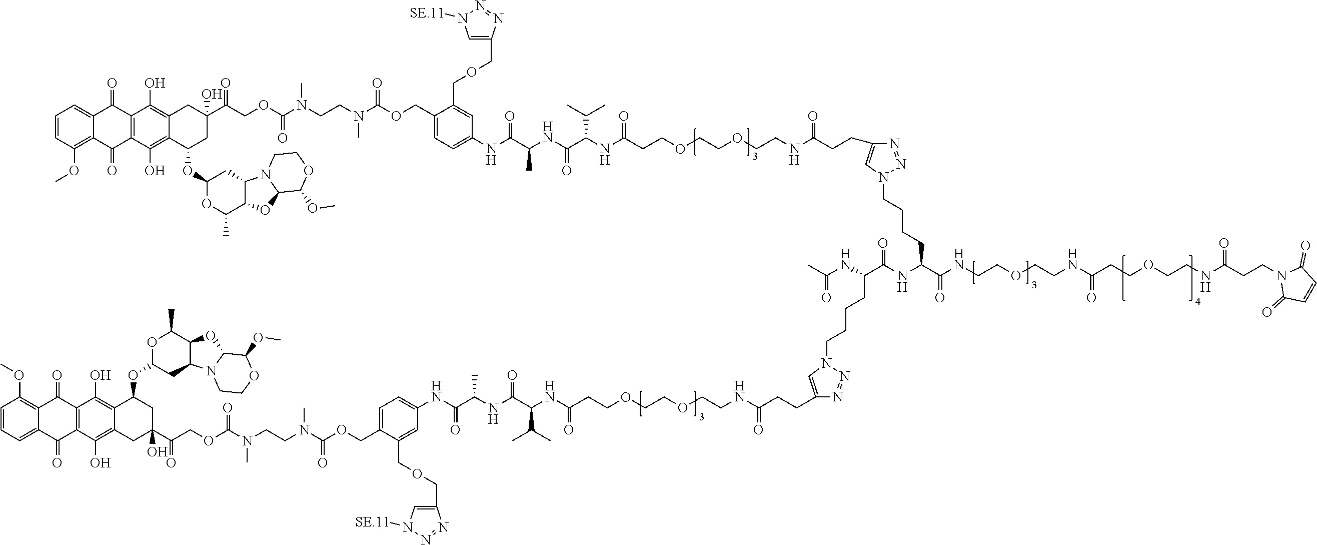

Compound IV.24 was synthesized as described for compound IV.16. Compound IV.24 (2.87 mg, 39%) was obtained as red solid. C₃₀₄H₄₅₈N₃₄O₁₅₂. UHPLC (method T): t_{R}=5.1 min. MS (ESI): found m/z=1755.0 [M+4H]⁴⁺, calcd. m/z=1755.23

### Example 27—Synthesis of IV.25

### Synthesis of I.25

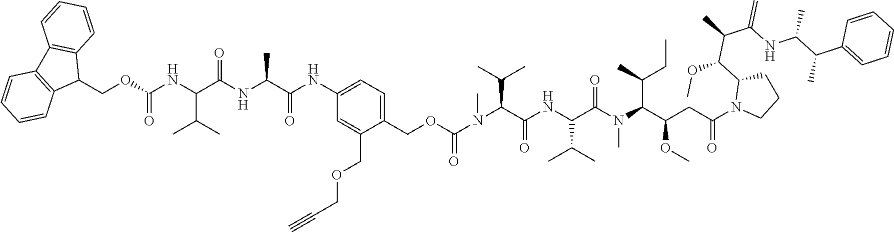

Intermediate I.25 was synthesized according to GP1 using Intermediate 4.3 (56.46 mg, 35.6 μmol, 1 equiv.), intermediate 1.6 (51.98 mg, 39.2 μmol, 1.1 equiv.), copper (II) sulfate pentahydrate (1.87 mg, 7.1 μmol, 0.2 equiv.), tris(benzyltriazolylmethyl)amine (4.7 mg, 8.9 μmol, 0.25 equiv.) and sodium ascorbate (3.5 mg, 17.8 μmol, 0.5 equiv.) in DMF/water (1188 μL, 10:1). Purification by RP-HPLC afforded intermediate 1.25 as colorless solid (69.98 mg, 68%) HPLC (method Q at 290 nm and TFA as additive): t_{R}=2.3 min. MS (ESI): found m/z=1328.9 [M+2H]²⁺, calcd. m/z=1327.75.

### Synthesis of II.25

Intermediate II.25 was synthesized according to GP3 using intermediate 1.25 (69.98 mg, 24.04 μmol, 1 equiv.), 59.4 μL diethylamine (577 μmol, 24 equiv.) in 3.5 mL DMF for Fmoc-deprotection and intermediate 3.2 (13 mg, 26.44 μmol, 1.1 equiv.), diisopropylethylamine (12.6 μL, 72.12 μmol, 3 equiv.) in DMF (350 μL) for the coupling reaction. Purification by RP-HPLC afforded intermediate II.25 (36 mg, 50%) as colorless solid. C₁₃₆H₂₂₃N₁₃O₆₁. HPLC (method Q with TFA as additive): t_{R}=2.0 min. MS (ESI): found m/z=1509.1 [M+2H]²⁺, calcd. m/z=1508.24

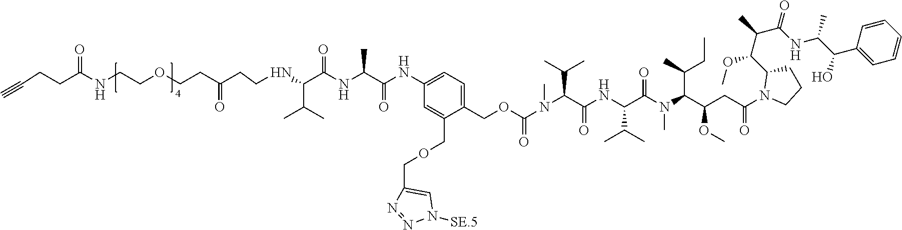

### Synthesis of III.25

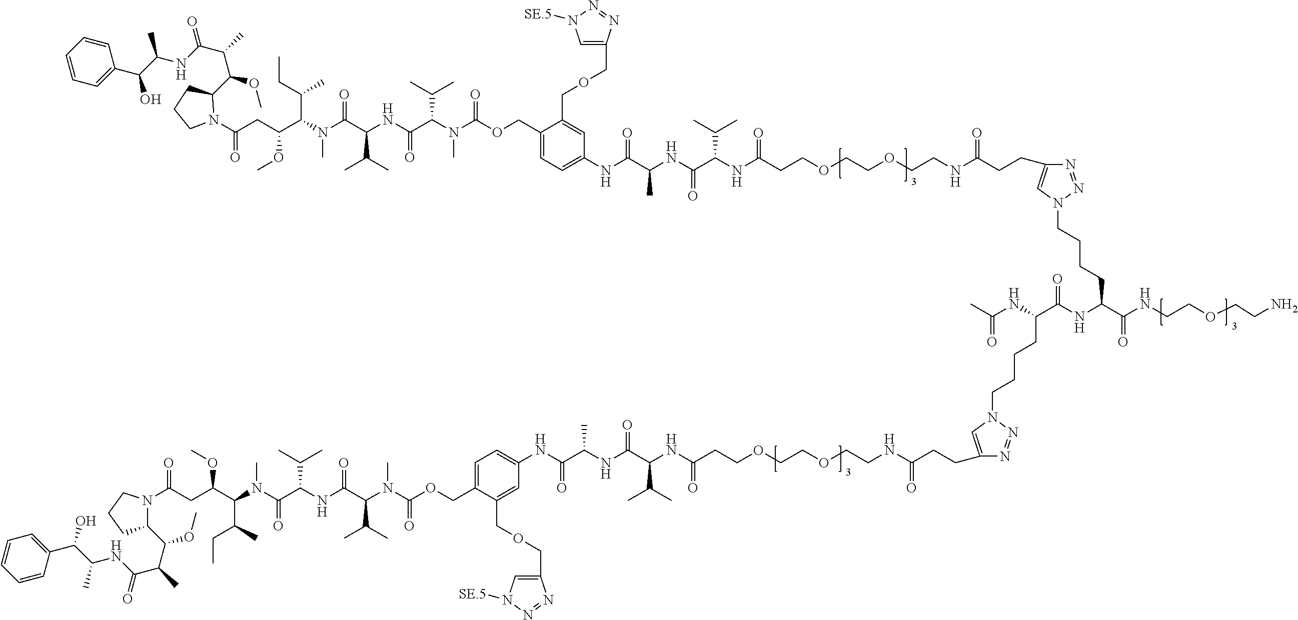

Intermediate III.25 was synthesized according to GP2 using intermediate 2.1 (1.53 mg, 2.66 μmol, 1 equiv.), intermediate II.25 (20 mg, 6.64 μmol, 2.5 equiv), copper (II) sulfate pentahydrate (0.8 mg, 3.32 μmol, 1.25 equiv.), Tris(3-hydroxypropyltriazolylmethyl)amine (2.89 mg, 6.64 μmol, 2.5 equiv.) and sodium ascorbate (1.97 mg, 9.96 μmol, 3.75 equiv.) in DMF/water (130.9 μL, 10:1). After 45 min the same amount of CuSO₄×5H₂O, THPTA, and Na-ascorbate was added to the reaction mixture. After 2.5 h the reaction was complete, and the reaction mixture was purified directly without any pretreatment by prep. HPLC to yield Intermediate III.25 as colorless lyophilizate (12.57 mg, 72%). C₂₉₄H₄₈₈N₃₆O₁₂₈. UHPLC (method U): t_{R}=2.8 min. MS (ESI): found m/z=1644.9 [M+4H]⁴⁺, calcd. m/z=1643.82

### Synthesis of IV.25

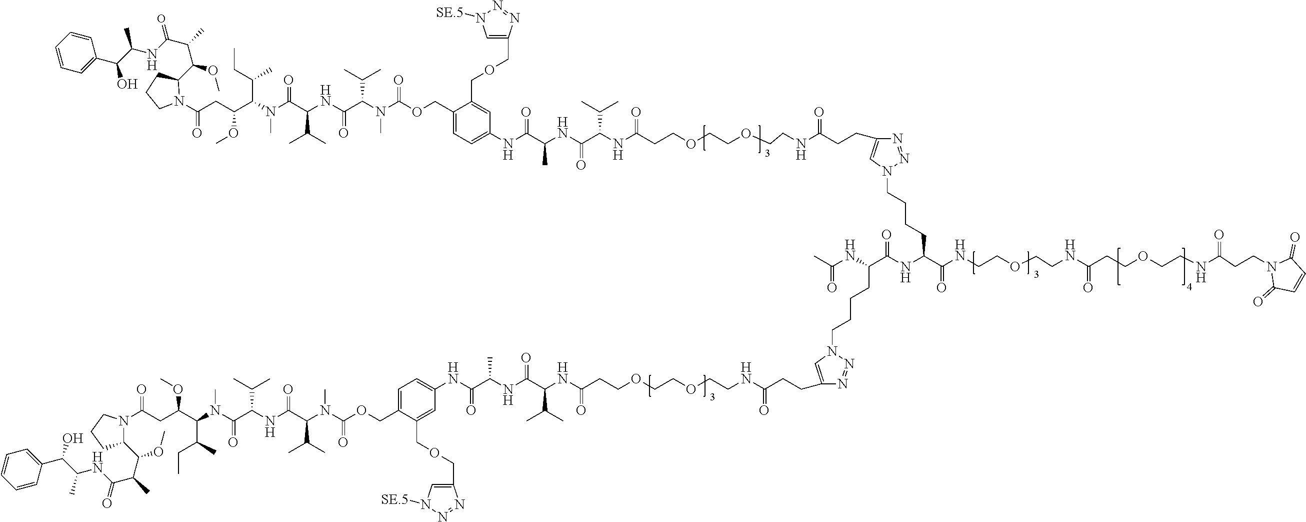

Compound IV.25 was synthesized as described for compound IV.16. Compound IV.25 (8.15 mg, 62%) was obtained as colorless solid. C₃₁₂H₅₁₄N₃₈O₁₃₆. UHPLC (method T): t_{R}=3.1 min. MS (ESI): found m/z=1744.6 [M+4H]⁴⁺, calcd. m/z=1744.12

### Example 28—Synthesis of IV.2

### Synthesis of I.26

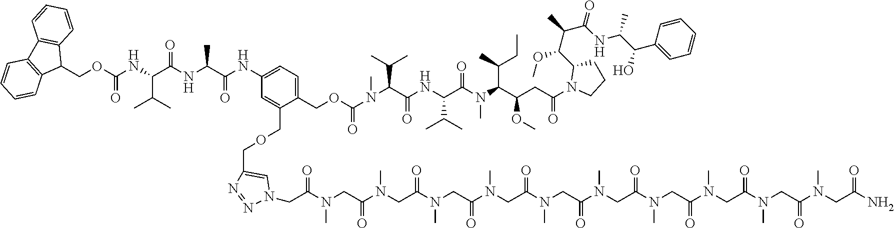 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](C)NC(=O)[C@@H](NC(=O)OCC2c3ccccc3-c3ccccc32)C(C)C)cc1COCc1cn(CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)nn1)C(C)C]

Intermediate I.26 was synthesized according to GP1 using intermediate 5.2 (29.3 mg, 35.8 μmol, 1 equiv.), intermediate 1.6 (52.3 mg, 39.4 μmol, 1.1 equiv.), copper (II) sulfate pentahydrate (1.79 mg, 7.2 μmol, 0.2 equiv.), tris(benzyltriazolylmethyl)amine (4.8 mg, 8.9 μmol, 0.25 equiv.) and sodium ascorbate (3.6 mg, 17.9 μmol, 0.5 equiv.) in DMF/water (1144 μL, 10:1). Purification by RP-HPLC afforded intermediate 1.26 as colorless solid (59.18 mg, 77%) HPLC (method Q at 290 nm and TFA as additive): t_{R}=2.4 min. MS (ESI): found m/z=1070.1 [M+2H]²⁺, calcd. m/z=1069.58

### Synthesis of II.26

Intermediate II.26 was synthesized according to GP3 using intermediate 1.26 (59.18 mg, 27.7 μmol, 1 equiv.), 68.4 μL diethylamine (664 μmol, 24 equiv.) in 3 mL DMF for Fmoc-deprotection and intermediate 3.2 (15.2 mg, 30.9 μmol, 1.1 equiv.), diisopropylethylamine (14.5 μL, 83 μmol, 3 equiv.) in DMF (296 μL) for the coupling reaction. Purification by RP-HPLC afforded intermediate II.26 (32.77 mg, 53%) as colorless solid. C₁₀₇H₁₇₁N₂₃O₂₉. HPLC (method U): t_{R}=2.7 min. MS (ESI): found m/z=1122.7 [M+2H]²⁺, calcd. m/z=1122.13

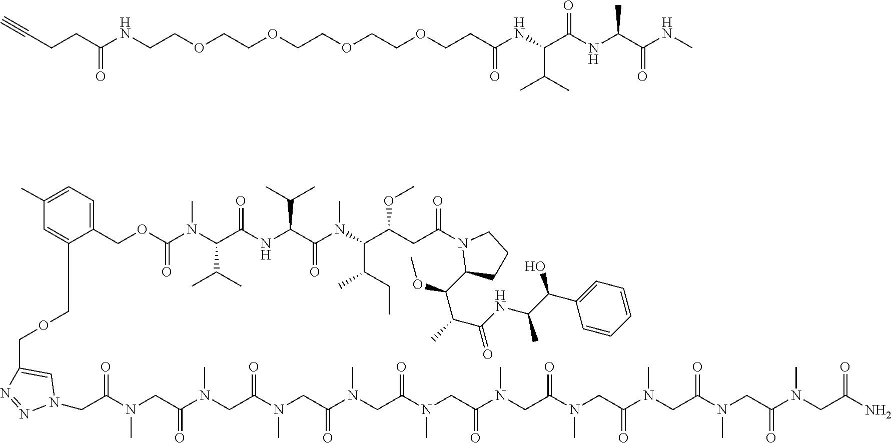 [SMILES: C#CCCC(=O)NCCOCCOCCOCCOCCC(=O)N[C@H](C(=O)N[C@@H](C)C(=O)NC)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(C)cc1COCc1cn(CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(=O)N(C)CC(N)=O)nn1)C(C)C]

### Synthesis of III.26

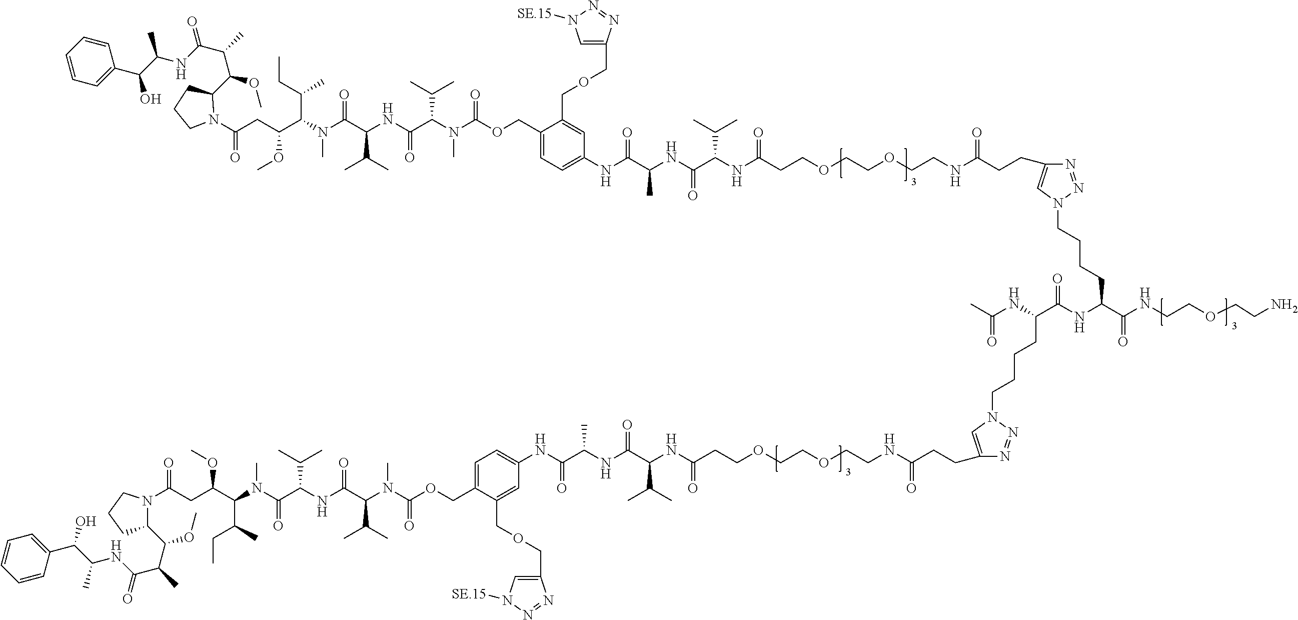

Intermediate III.26 was synthesized according to GP2 using intermediate 2.1 (3.36 mg, 5.8 μmol, 1 equiv.), intermediate II.26 (32.5 mg, 14.5 μmol, 2.5 equiv.), copper (II) sulfate pentahydrate (1.81 mg, 7.25 μmol, 1.25 equiv.), Tris(3-hydroxypropyltriazolylmethyl)amine (6.3 mg, 14.5 μmol, 2.5 equiv.) and sodium ascorbate (4.3 mg, 21.7 μmol, 3.75 equiv.) in DMF/water (286 μL, 10:1). After 30 min the same amount of CuSO₄×5H₂O, THPTA, and Na-ascorbate was added to the reaction mixture. After 1.5 h the reaction was complete, and the reaction mixture was purified directly without any pretreatment by prep. HPLC to yield Intermediate III.26 as colorless lyophilizate (22.9 mg, 78%). C₂₃₆H₃₈₄N₅₆O₆₄. UHPLC (method U): t_{R}=2.9 min. MS (ESI): found m/z=1258.4 [M+4H]⁴⁺, calcd. m/z=1257.71

### Synthesis of IV.26

Compound IV.26 was synthesized as described for compound IV.16. Compound IV.26 (18.37 mg, 76%) was obtained as colorless solid. C₂₅₄H₄₁₀N₅₈O₇₂. UHPLC (method U): t_{R}=3.3 min. MS (ESI): found m/z=1358.2 [M+4H]⁴⁺, calcd. m/z=1358.01

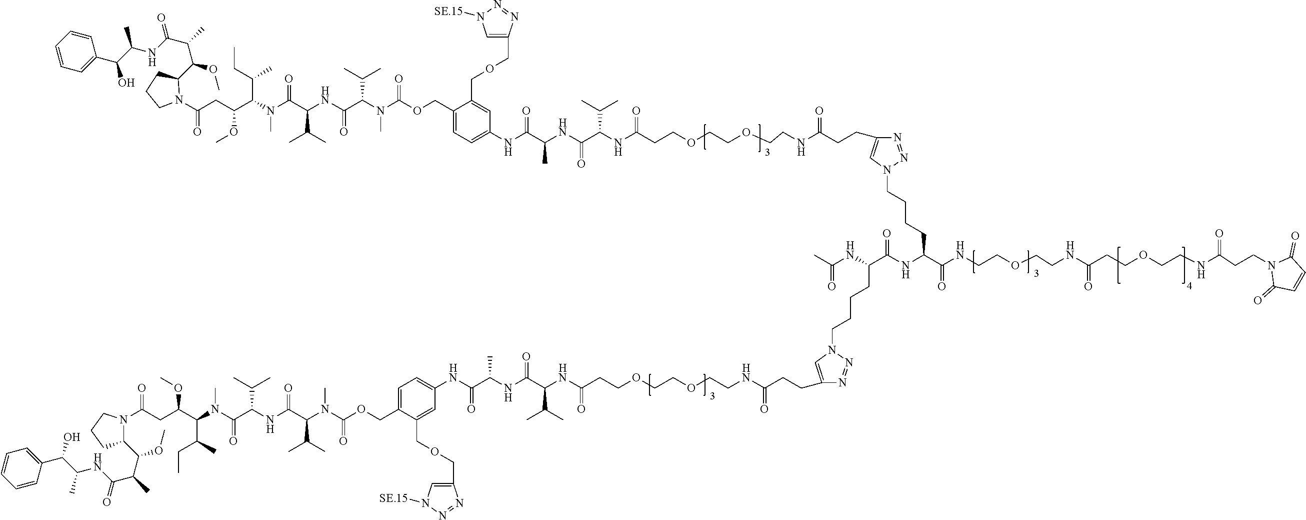

### Example 29—Synthesis of IV.27

### Synthesis of I.27

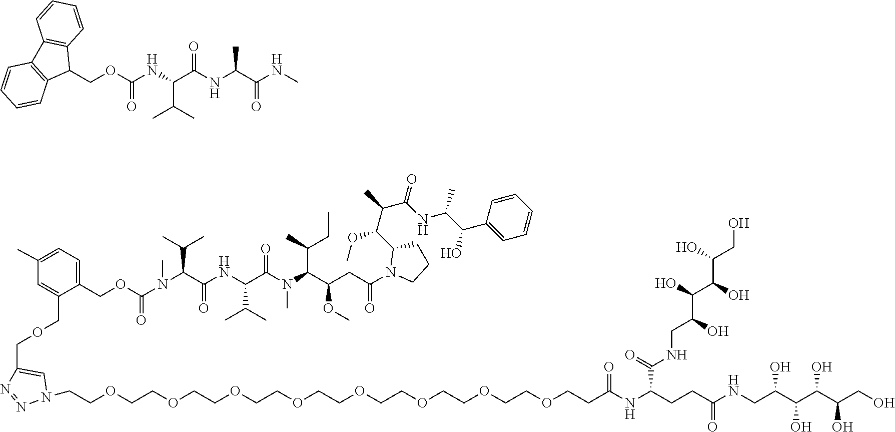 [SMILES: CNC(=O)[C@H](C)NC(=O)[C@@H](NC(=O)OCC1c2ccccc2-c2ccccc21)C(C)C ; CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(C)cc1COCc1cn(CCOCCOCCOCCOCCOCCOCCOCCOCCC(=O)N[C@@H](CCC(=O)NC[C@H](O)[C@@H](O)[C@H](O)[C@H](O)CO)C(=O)NC[C@H](O)[C@@H](O)[C@H](O)[C@H](O)CO)nn1)C(C)C]

Intermediate I.27 was synthesized according to GP1 using Intermediate 6.4 (35.5 mg, 38.3 μmol, 1 equiv.), intermediate 1.6 (55.5 mg, 41.8 μmol, 1.1 equiv.), copper (II) sulfate pentahydrate (1.9 mg, 7.6 μmol, 0.2 equiv.), tris(benzyltriazolylmethyl)amine (5 mg, 9.5 μmol, 0.25 equiv.) and sodium ascorbate (3.8 mg, 19 μmol, 0.5 equiv.) in DMF/water (1221 μL, 10:1). Purification by RP-HPLC afforded intermediate 1.27 as colorless solid (68.01 mg, 80%) HPLC (method Q at 290 nm and TFA as additive): t_{R}=2.4 min. MS (ESI): found m/z=1126.2 [M+2H]²⁺, calcd. m/z=1125.61

### Synthesis of II.27

Intermediate II.27 was synthesized according to GP3 using intermediate 1.27 (68.01 mg, 30.2 μmol, 1 equiv.), 74.7 μL diethylamine (725.3 μmol, 24 equiv.) in 3.4 mL DMF for Fmoc-deprotection and intermediate 3.2 (16.3 mg, 33.24 μmol, 1.1 equiv.), diisopropylethylamine (15.8 μL, 90.66 μmol, 3 equiv.) in DMF (340 μL) for the coupling reaction. Purification by RP-HPLC afforded intermediate II.27 (30.1 mg, 42%) as colorless solid. C₁₁₁H₁₈₇N₁₅₀₃₉. HPLC (method U): t_{R}=2.7 min. MS (ESI): found m/z=1178.7 [M+2H]²⁺, calcd. m/z=1178.16

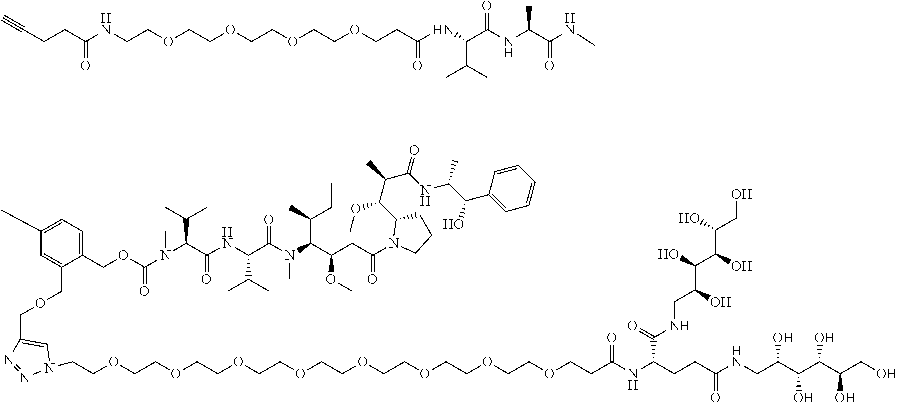

### Synthesis of III.27

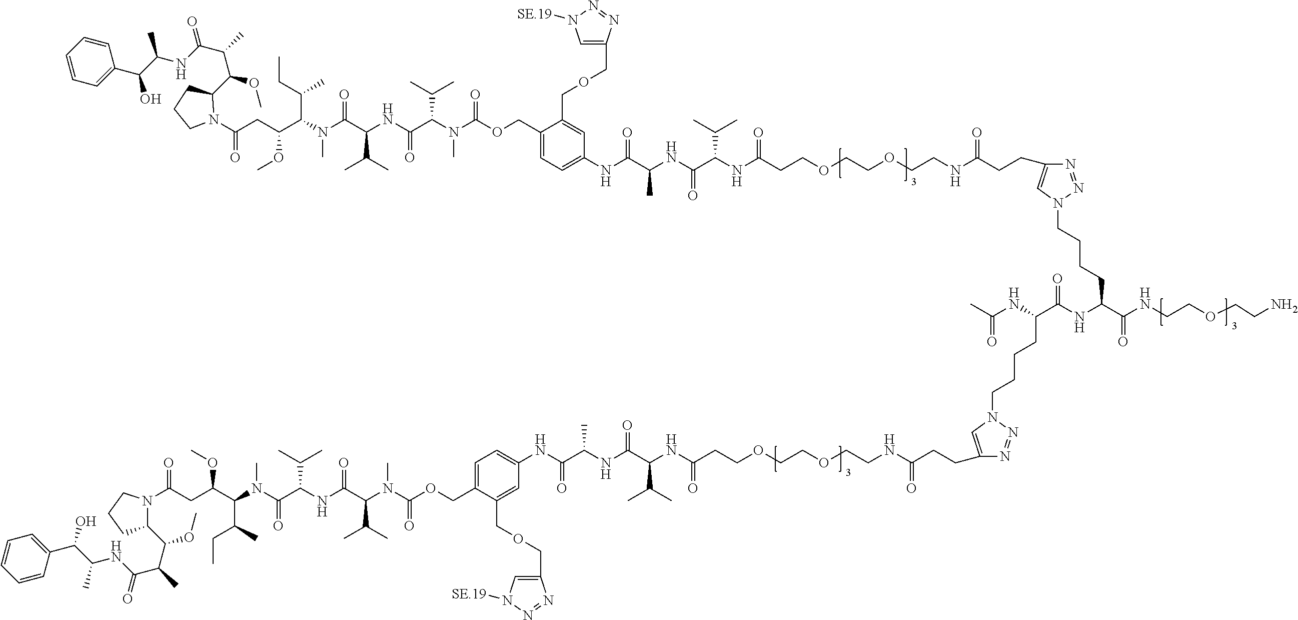

Intermediate III.27 was synthesized according to GP2 using intermediate 2.1 (3 mg, 5.18 μmol, 1 equiv.), intermediate II.27 (30.4 mg, 12.9 μmol, 2.5 equiv.), copper (II) sulfate pentahydrate (1.62 mg, 6.47 μmol, 1.25 equiv.), Tris(3-hydroxypropyltriazolylmethyl)amine (5.61 mg, 12.9 μmol, 2.5 equiv.) and sodium ascorbate (3.84 mg, 19.4 μmol, 3.75 equiv.) in DMF/water (130.9 μL, 10:1). After 30 min the same amount of CuSO₄×5H₂O, THPTA, and Na-ascorbate was added to the reaction mixture. After 1.5 h the reaction was complete, and the reaction mixture was purified directly without any pretreatment by prep. HPLC to yield intermediate III.27 as colorless lyophilizate (21.06 mg, 77%). C₂₉₄H₄₈₈N₃₆O₁₂₈. UHPLC (method U): t_{R}=2.9 min. MS (ESI): found m/z=1178.7 [M+4H]⁴⁺, calcd. m/z=1178.16

### Synthesis of IV.27

Compound IV.27 was synthesized as described for compound IV.16. Compound IV.27 (15.92 mg, 72%) was obtained as colorless solid. C₂₆₂H₄₄₂N₄₂O₉₂. UHPLC (method U): t_{R}=3.3 min. MS (ESI): found m/z=1414.2 [M+4H]⁴⁺, calcd. m/z=1414.04

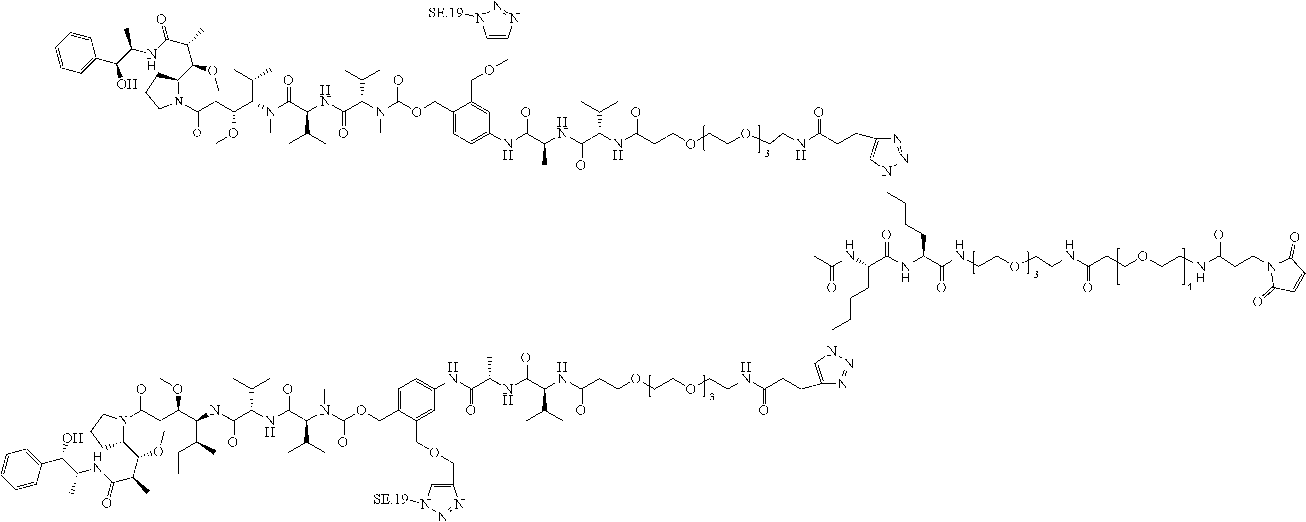

### Example 30—Synthesis of IV.28

### Synthesis of I.28

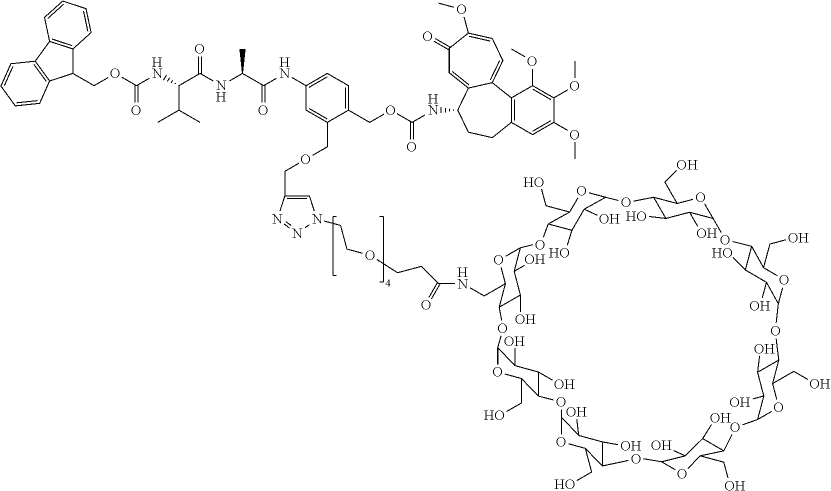

Intermediate I.28 was synthesized according to intermediate 1.24 using Intermediate 4.6 (268.92 mg, 171 μmol, 1 equiv.), intermediate 1.7 (182.24 mg, 188 μmol, 1.1 equiv.), copper (II) sulfate pentahydrate (8.1 mg, 34.2 μmol, 0.2 equiv.), tris(benzyltriazolylmethyl)amine (22.76 mg, 42.8 μmol, 0.25 equiv.) and sodium ascorbate (17 mg, 85.5 μmol, 0.5 equiv.) in DMF/water (4004 μL, 10:1). After 20 min the reaction mixture was precipitated in ice cold acetone to yield intermediate 1.28. The crude product (489.62 mg with a purity of 91%) was used without further purification. UHPLC (method R with 0.05% TFA as additive): t_{R}=2.0 min. MS (ESI): found m/z=1268.2 [M+2H]²⁺, calcd. m/z=1268.49.

### Synthesis of II.28

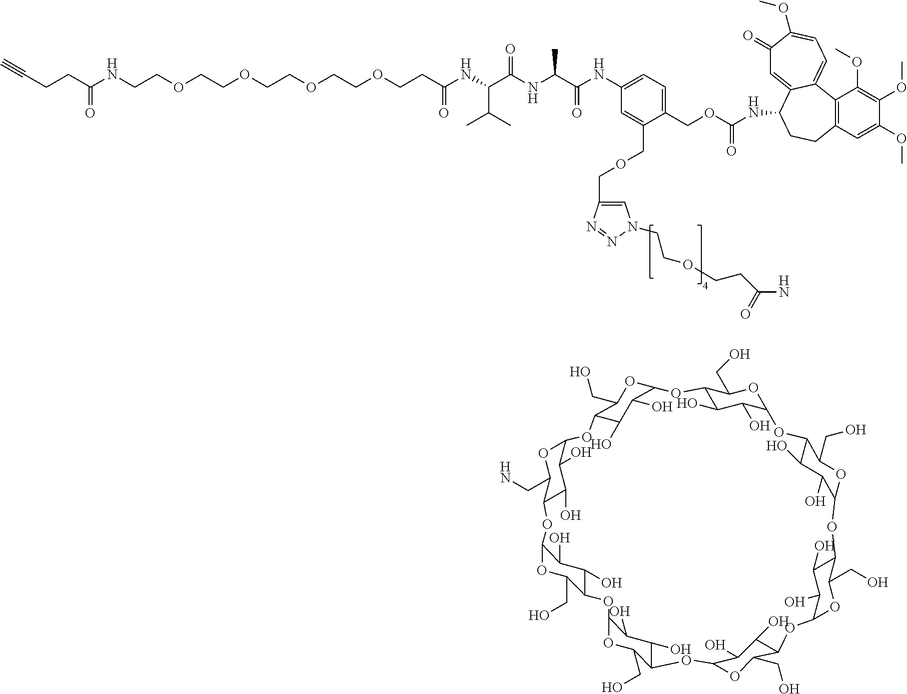

Intermediate II.28 was synthesized according to GP3 using intermediate 1.28 (200.52 mg, 39.4 μmol, 1 equiv.), 97.6 μL diethylamine (946 μmol, 24 equiv.) in 1 mL DMF for Fmoc-deprotection and intermediate 3.2 (21.2 mg, 43.3 μmol, 1.1 equiv.), diisopropylethylamine (20.6 μL, 1.2 mmol, 3 equiv.) in DMF (500 μL) for the coupling reaction. Purification by RP-HPLC afforded intermediate II.28 (93.5 mg, 50%) as yellow solid. C₁₃₆H₂₂₃N₁₃O₆₁. HPLC (method W): t_{R}=1.9 min. MS (ESI): found m/z=1320.5 [M+2H]²⁺, calcd. m/z=1321.04

### Synthesis of III.28

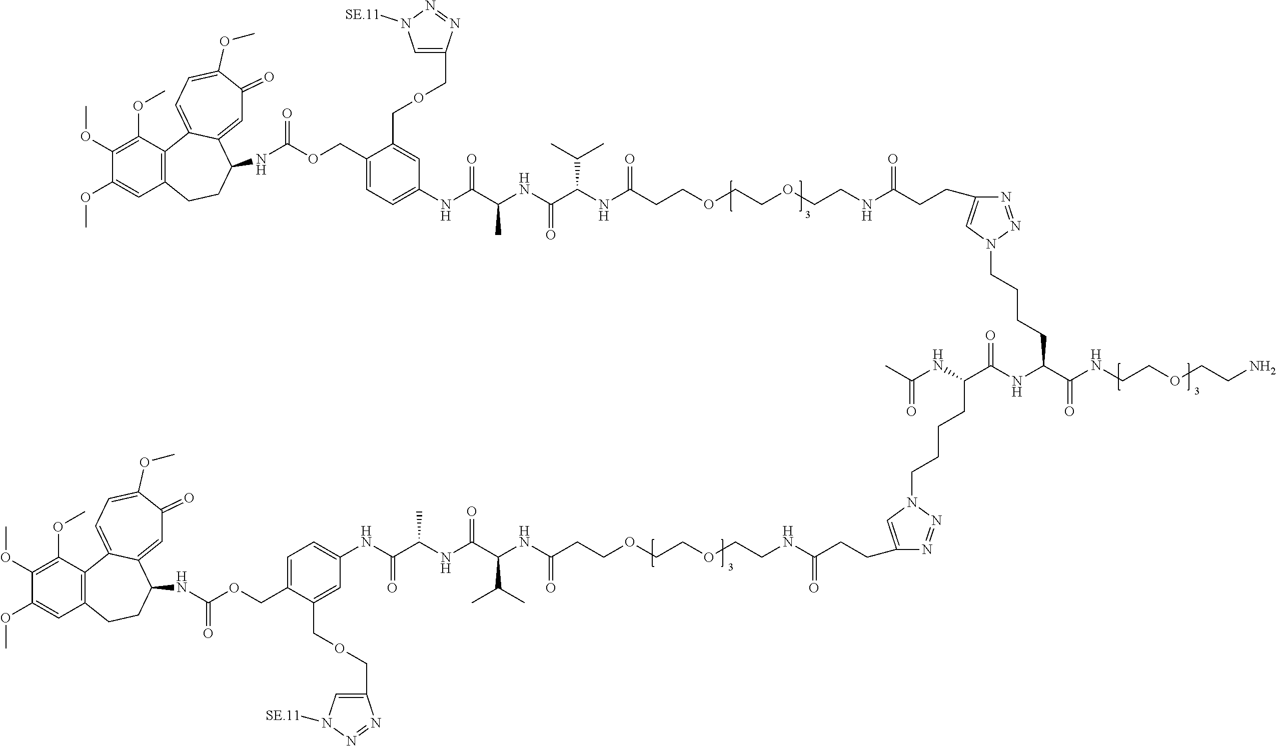

Intermediate III.28 was synthesized according to GP2 using intermediate 2.1 (3.37 mg, 5.8 μmol, 1 equiv.), intermediate II.28 (33.78 mg, 12.8 μmol, 2.2 equiv.), copper (II) sulfate pentahydrate (1.82 mg, 7.3 μmol, 1.25 equiv.), Tris(3-hydroxypropyltriazolylmethyl)amine (6.3 mg, 14.5 μmol, 2.5 equiv.) and sodium ascorbate (4.32 mg, 21.8 μmol, 3.75 equiv.) in DMF/water (130.9 μL, 10:1). After 30 min the same amount of CuSO₄×5H₂O, THPTA, and Na-ascorbate was added to the reaction mixture. After 2 h the reaction was complete, and the reaction mixture was purified directly without any pretreatment by prep. HPLC to yield Intermediate III.28 as colorless lyophilizate (21.56 mg, 67%). C₂₅₂H₃₈₈N₂₈O₁₂₆. UHPLC (method T with 0.05% TFA as additive at 380 nm): t_{R}=3.4 min. MS (ESI): found m/z=1456.4 [M+4H]⁴⁺, calcd. m/z=1456.62

### Synthesis of IV.28

Compound IV.28 was synthesized as described for compound IV.16. Compound IV.28 (17.2 mg, 76%) was obtained as yellow solid. C₂₇₀H₄₁₄N₃₀O₁₃₄. UHPLC (method T): t_{R}=3.9 min. MS (ESI): found m/z=1555.9 [M+4H]⁴⁺, calcd. m/z=1556.16

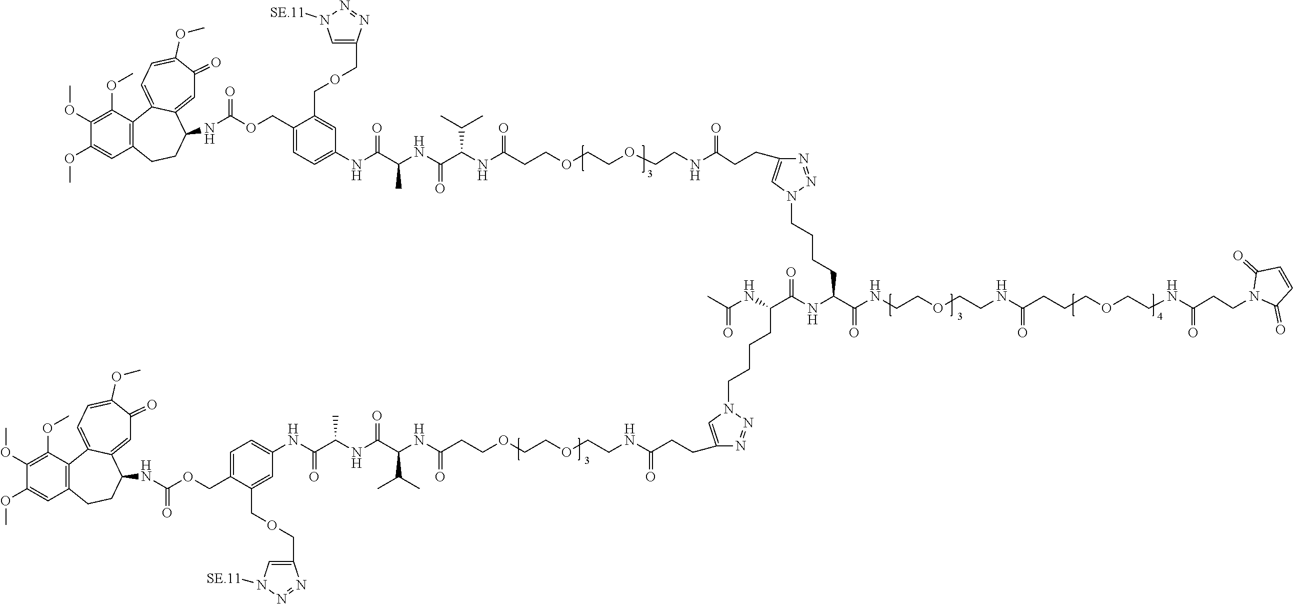

### Example 31—Synthesis of IV.29

### Synthesis of III.29

Intermediate III.29 was synthesized according to GP2 using intermediate 2.2 (4.4 mg, 5.3 μmol, 1 equiv.), intermediate II.28 (60.12 mg, 23.2 μmol, 4.4 equiv.), copper (II) sulfate pentahydrate (3.2 mg, 13.2 μmol, 2.5 equiv.), Tris(3-hydroxypropyltriazolylmethyl)amine (11.3 mg, 26.4 μmol, 5 equiv.) and sodium ascorbate (7.65 mg, 39.6 μmol, 7.5 equiv.) in DMF/water (130.9 μL, 10:1). After 30 min the same amount of CuSO₄×5H₂O, THPTA, and Na-ascorbate was added to the reaction mixture. After 2 h the reaction was complete, and the reaction mixture was purified directly without any pretreatment by prep. HPLC to yield Intermediate III.29 as pale yellow lyophilizate (32.58 mg, 67%). C₄₉₄H₇₅₄N₅₄O₂₅₅. UHPLC (method T with 0.05% TFA as additive at 380 nm): t_{R}=4.15 min. MS (ESI): found m/z=1902.7 [M+6H]⁶⁺, calcd. m/z=1902.8

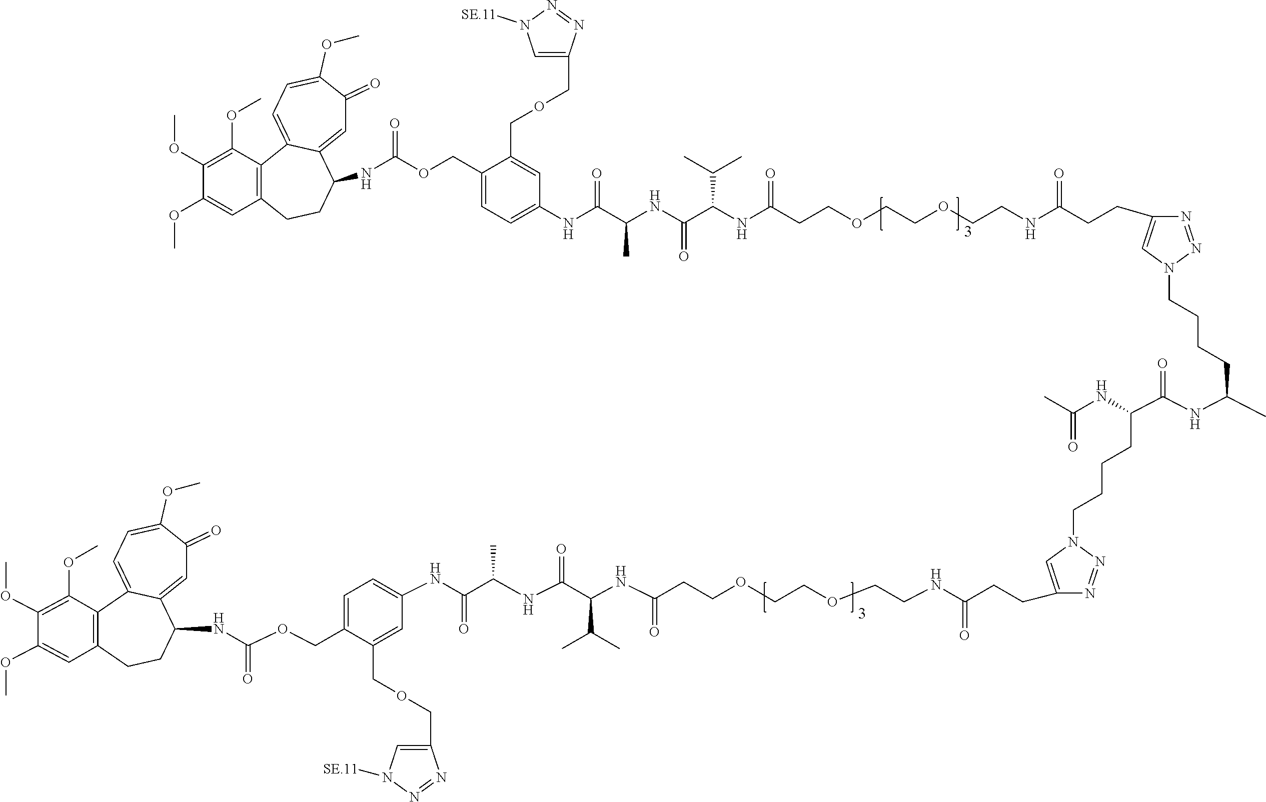

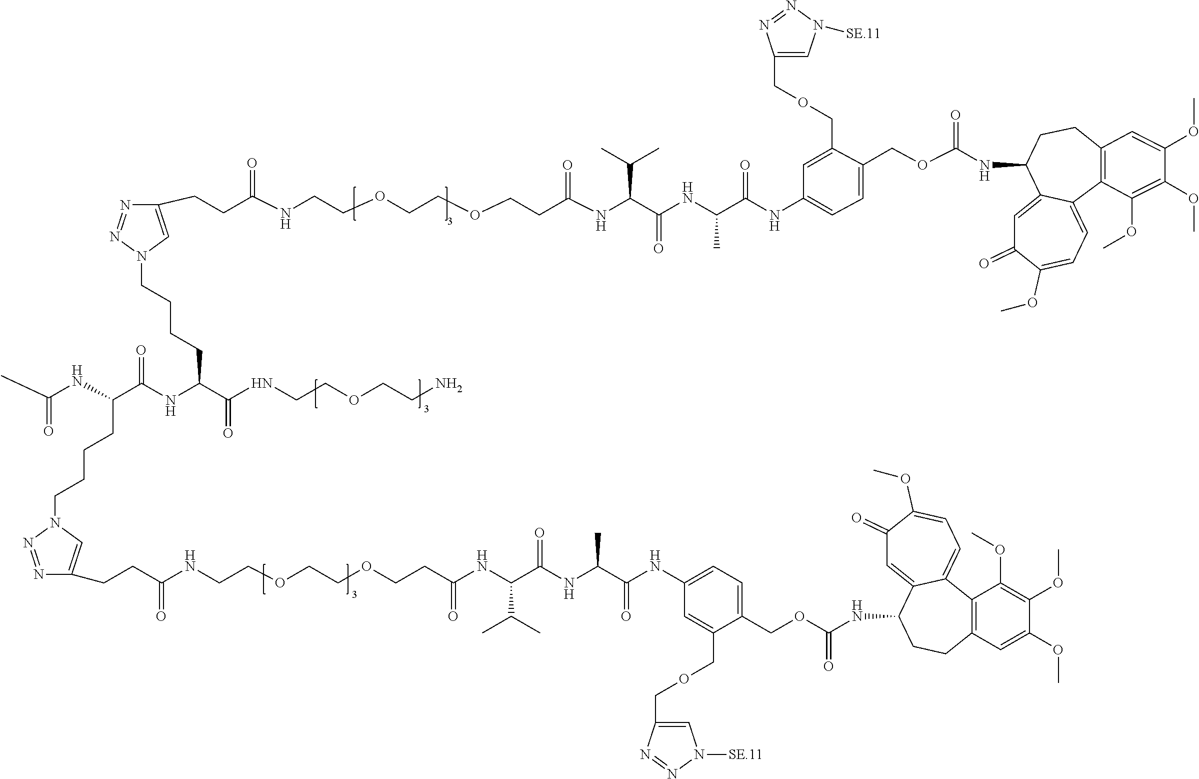

### Synthesis of IV.29

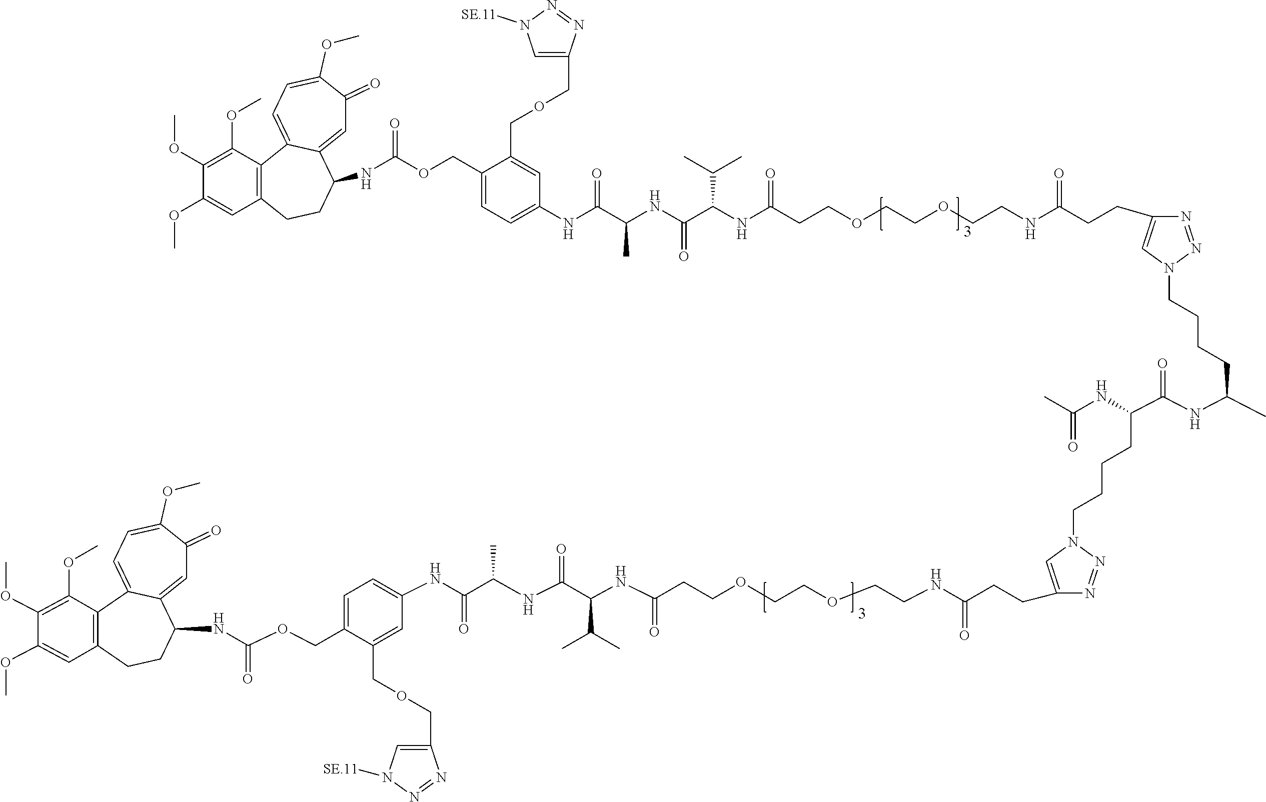

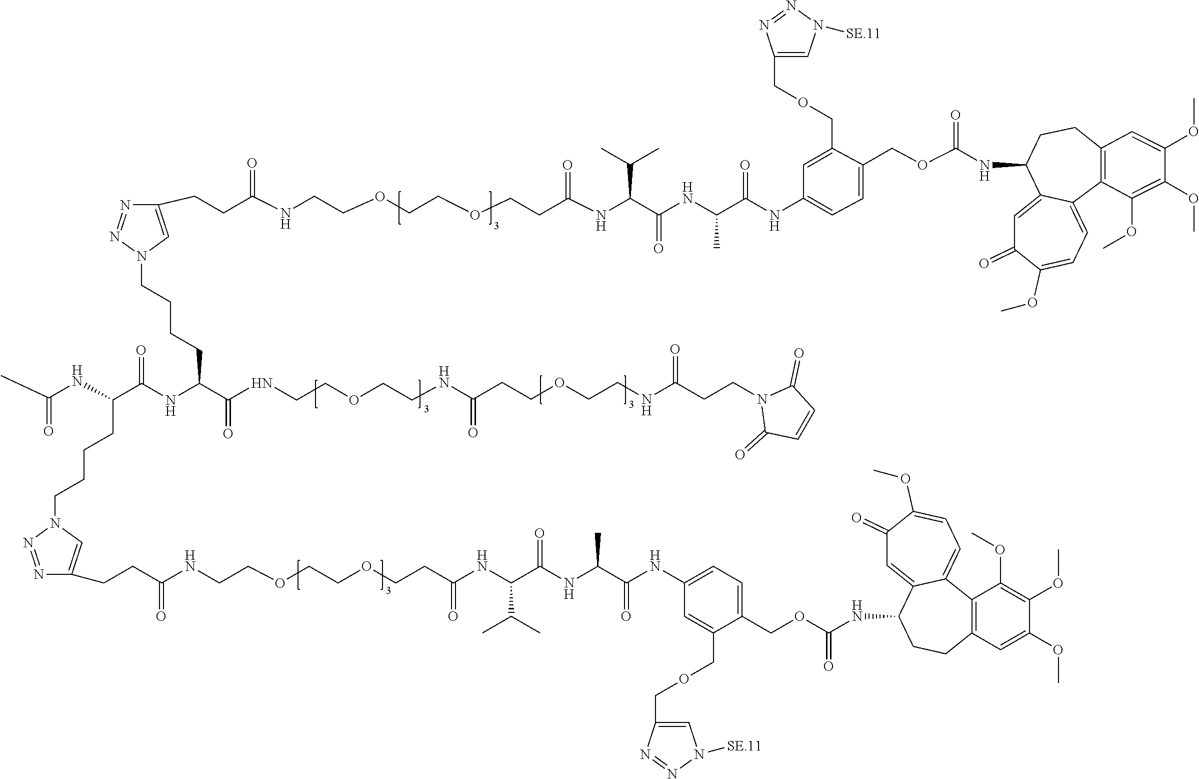

Compound IV.29 was synthesized as described for compound IV.16. Compound IV.29 (22.37 mg, 67%) was obtained as pale yellow solid. C₅₁₂H₇₈₀N₅₆O₂₅₆. UHPLC (method T): t_{R}=4.4 min. MS (ESI): found m/z=1969.0 [M+6H]⁶⁺, calcd. m/z=1969.16

### Synthesis of I.30

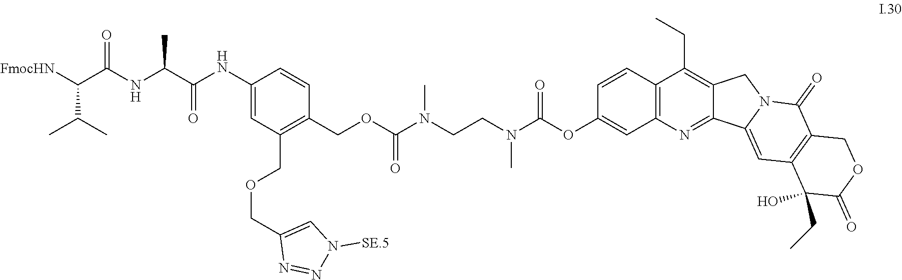

Intermediate I.30 was synthesized according to GP1 using intermediate 4.3 (59.7 mg, 38 μmol, 1 equiv.), intermediate 1.17 (42.1 mg, 38 μmol, 1 equiv.), copper (II) sulfate pentahydrate (4.7 mg, 19 μmol, 0.5 equiv.), tris(benzyltriazolylmethyl)amine (20.0 mg, 38 μmol, 1 equiv.) and sodium ascorbate (11.9 mg, 57 μmol, 1.5 equiv.) in DMF/water (4.05 mL, 20:1). Purification by RP-HPLC afforded intermediate 1.30 (100.4 mg, 99%) as yellowish solid. HPLC (method A): t_{R}=10.0 min. MS (ESI): found m/z=1350.3 [M+2H]²⁺, calcd. m/z=1350.0 [M+2H]²⁺.

### Synthesis of II.30

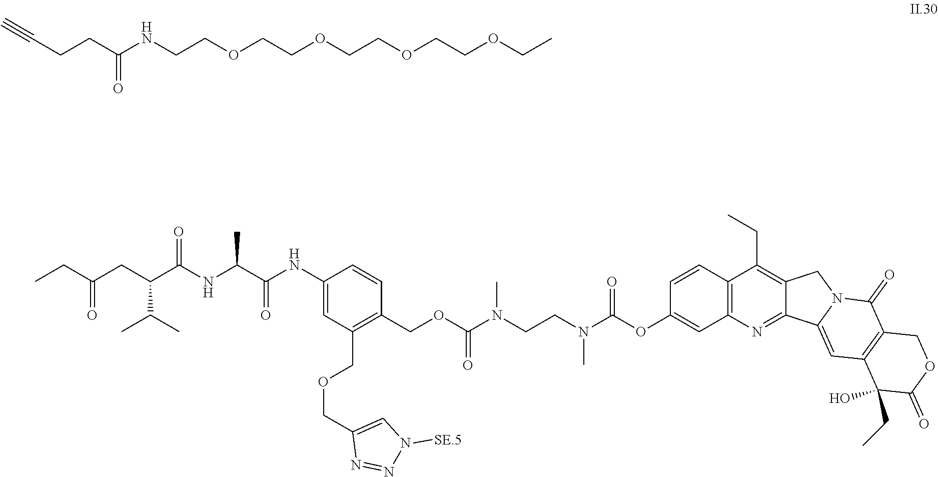

Intermediate II.30 was synthesized according to GP3 using 15.5 μl diethylamine (149 μmol, 4 equiv.) in 3.2 mL DMF for Fmoc-deprotection of intermediate 1.30 (100.4 mg, 37 μmol, 1 equiv.) and intermediate 3.2 (23.7 mg, 48 μmol, 1.3 equiv.), diisopropylethylamine (9.07 μL, 52 μmol, 1.4 equiv.) in DMF for the coupling reaction. Purification by RP-HPLC afforded intermediate II.30 (63.8 mg, 61%) as yellowish solid. HPLC (method N): t_{R}=14.1 min. MS (ESI): found m/z=1424.9 [M+2Na]²⁺, calcd. m/z=1424.6 [M+2Na]²⁺.

### Synthesis of III.30

Intermediate III.30 was synthesized according to GP2 using intermediate 2.1 (6.59 mg, 11 μmol, 1 equiv.), intermediate II.30 (63.8 mg, 23 μmol, 2 equiv), copper (II) sulfate pentahydrate (3.41 mg, 14 μmol, 1.2 equiv.), Tris(3-hydroxypropyltriazolylmethyl)amine (11.86 mg, 27 μmol, 2.4 equiv.) and sodium ascorbate (8.1 mg, 41 μmol, 3.6 equiv.) in DMF/water (0.42 mL, 5:1). Purification by RP-HPLC afforded intermediate III.30 (32.2 mg, 45%) as yellowish solid. C₂₇₀H₄₁₄N₃₄O₁₂₆. HPLC (method AD): t_{R}=20.9 min. MS (ESI): found m/z=1538.9 [M+4H]⁴⁺, calcd. m/z=1538.2 [M+4H]⁴⁺.

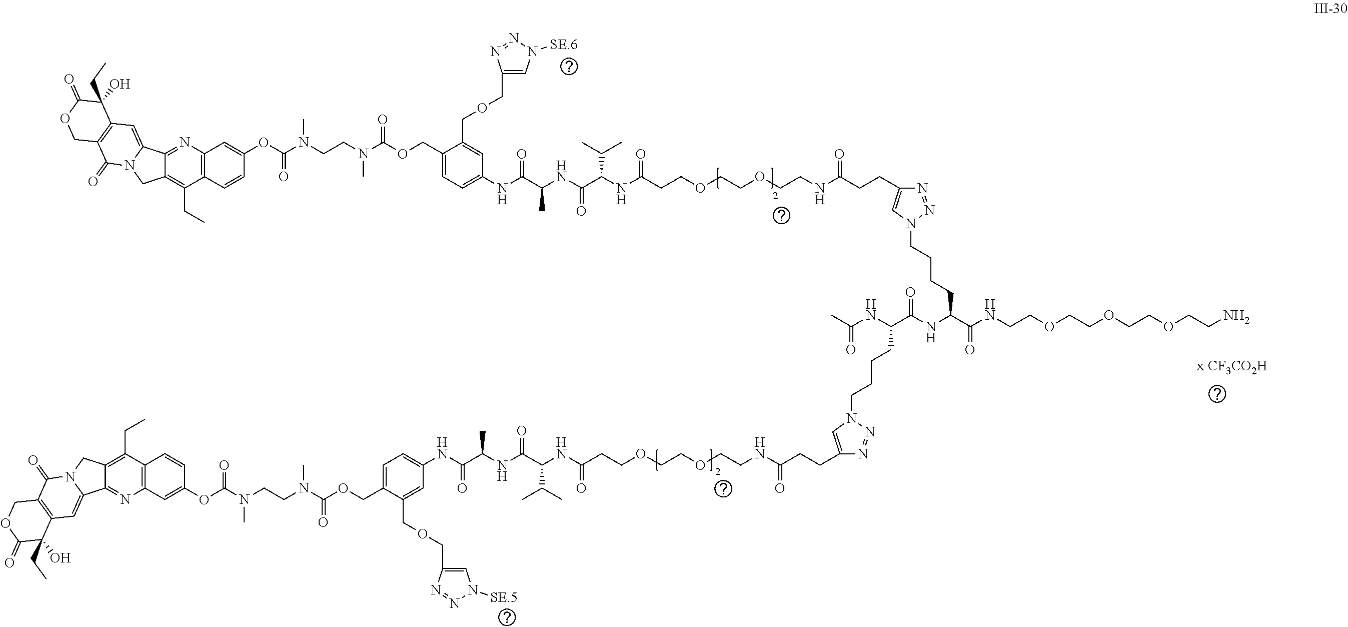

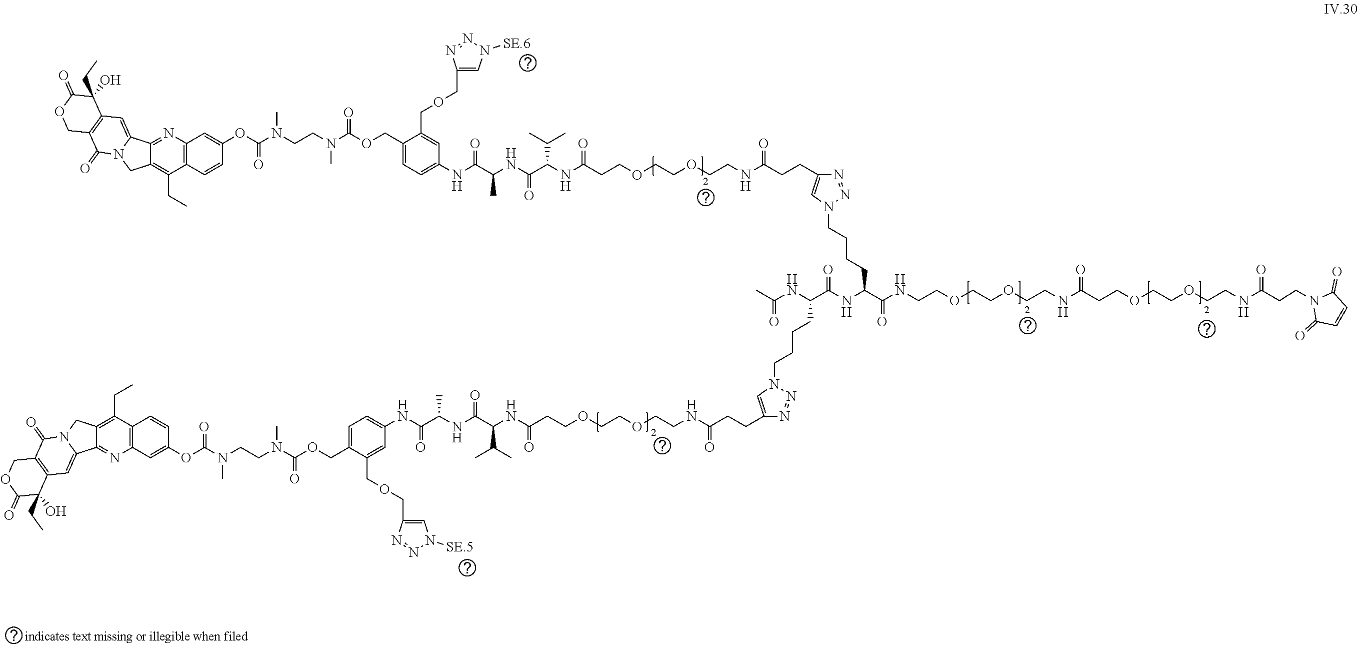

### Synthesis of IV.30

Compound IV.30 was synthesized according to GP4 using the TFA-salt of intermediate III.30 (31.5 mg, 5 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (5.17 mg, 10.1 μmol, 2 equiv.) and DIPEA (1.75 μL, 10.1 μmol, 2 equiv.) in DMF (0.87 mL). Purification by RP-HPLC afforded compound IV.30 (24.5 mg, 74%) as yellowish solid. C₂₈₈H₄₄₀N₃₆O₁₃₄. HPLC (method J): t_{R}=13.2 min. MS (ESI): found m/z=1092.4 [M+6H]⁶⁺, calcd. m/z=1092.1 [M+6H]⁶⁺.

### Synthesis of IV.31

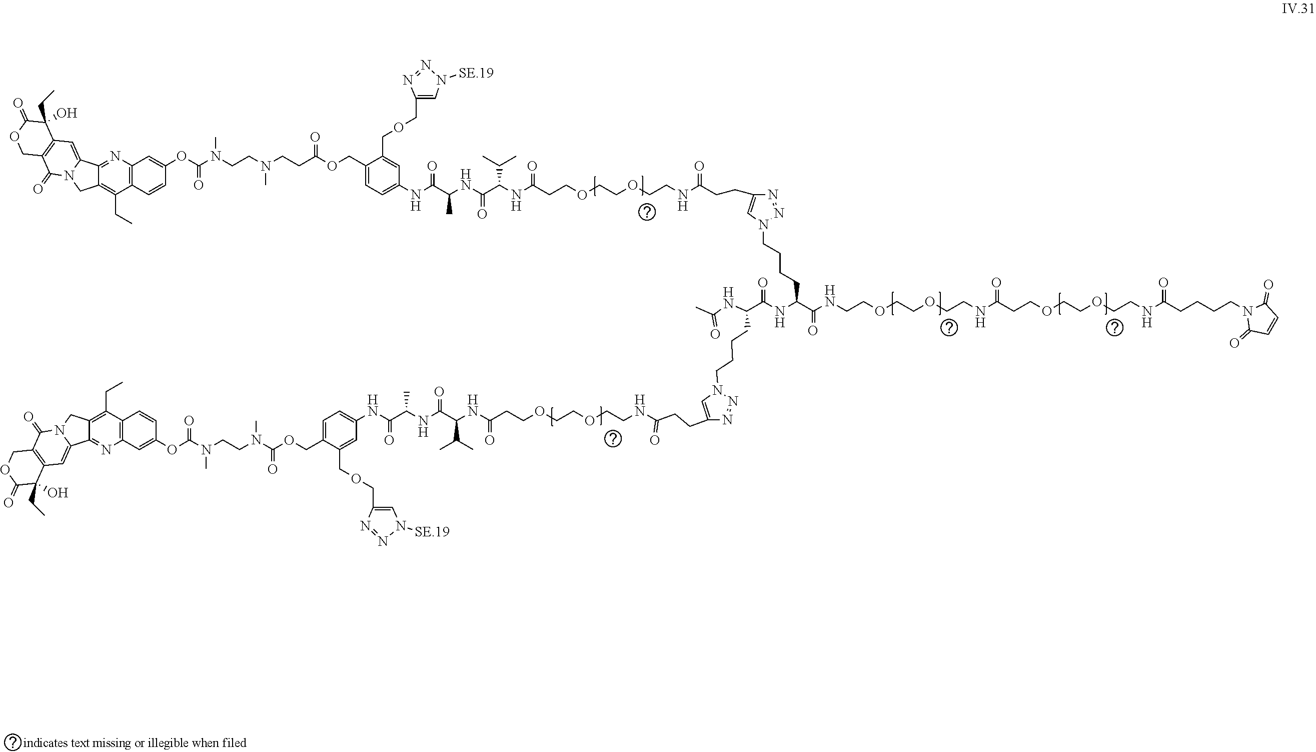

Compound IV.31 was synthesized as described for compound IV.30 but using intermediate 6.4. Compound IV.31 (32.76 mg, 67%) was obtained as yellowish solid. C₂₃₈H₃₆₈N₄₀O₉₀. HPLC (method AD): t_{R}=23.0 min. MS (ESI): found m/z=1329.5 [M+4Na]⁴⁺, calcd. m/z=1329.6 [M+4Na]⁴⁺.

### Synthesis of I.32

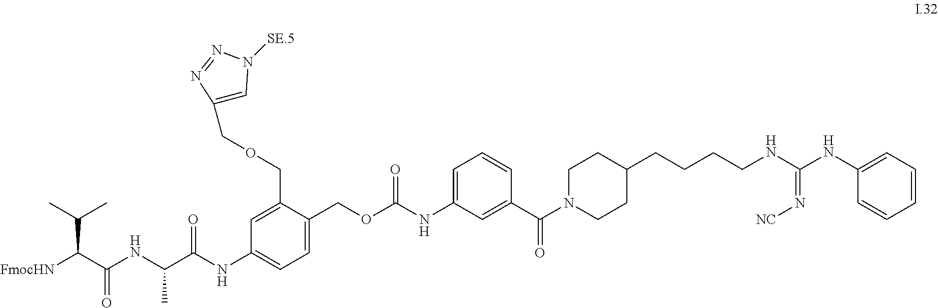

Intermediate I.32 was synthesized according to GP1 using intermediate 4.3 (76.9 mg, 48.5 μmol, 1 equiv.), intermediate 1.18 (50 mg, 48.5 μmol, 1 equiv.), copper (II) sulfate pentahydrate (2.4 mg, 9.7 μmol, 0.2 equiv.), tris(benzyltriazolylmethyl)amine (6.4 mg, 12.1 μmol, 0.25 equiv.) and sodium ascorbate (11.5 mg, 58.3 μmol, 1.2 equiv.) in DMF/water (769 μL, 10:1). Purification by RP-HPLC afforded intermediate 1.32 (70 mg, 55%) as colorless solid. HPLC (method X): t_{R}=8.3 min. MS (ESI): found m/z=1306.2 [M+2H]²⁺, calcd. m/z=1306.6 [M+2H]²⁺.

### Synthesis of II.32

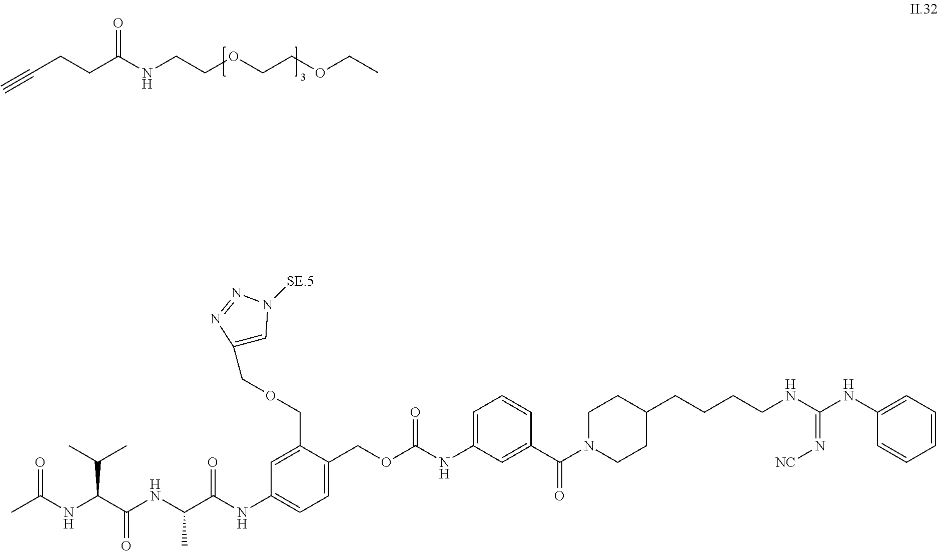

Intermediate II.32 was synthesized according to GP3 using 66.6 μL diethylamine (640 μmol, 24 equiv.) in 3.5 mL DMF for Fmoc-deprotection of intermediate 1.32 (70 mg, 26.8 μmol, 1 equiv.) and intermediate 3.2 (14.5 mg, 29.5 μmol, 1.1 equiv.), diisopropylethylamine (4.66 μL, 26.8 μmol, 1 equiv.) in DMF (1.75 mL) for the coupling reaction. Purification by RP-HPLC afforded intermediate II.32 (55.5 mg, 76%) as colorless solid. HPLC (method X): t_{R}=5.9 min. MS (ESI): found m/z=1359.0 [M+2H]²⁺, calcd. m/z=1359.1 [M+2H]²⁺.

### Synthesis of III.32

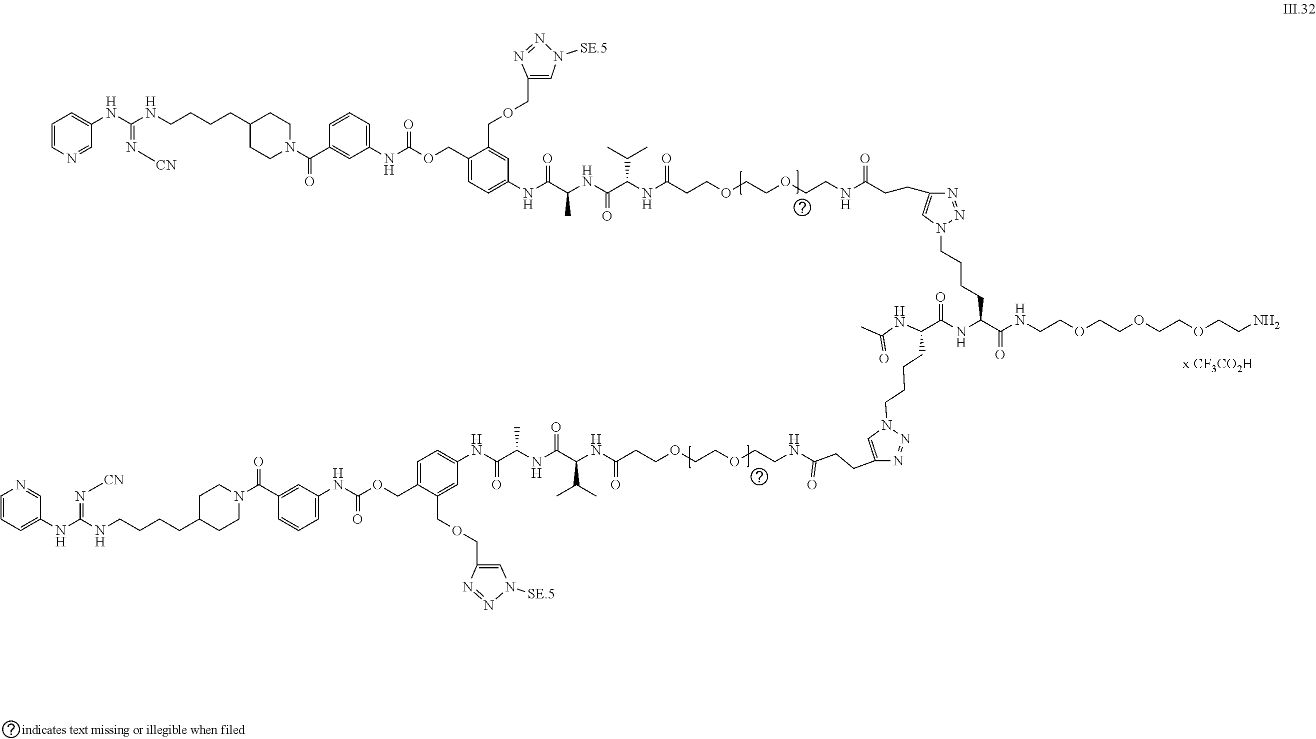

Intermediate III.32 was synthesized according to GP2 using intermediate 2.1 (5.14 mg, 8.9 μmol, 1 equiv.), intermediate II.32 (55.53 mg, 20.4 μmol, 2.3 equiv), copper (II) sulfate pentahydrate (2.77 mg, 11.1 μmol, 1.25 equiv.), Tris(3-hydroxypropyltriazolylmethyl)amine (9.7 mg, 22.3 μmol, 2.5 equiv.) and sodium ascorbate (6.6 mg, 33.2 μmol, 3.75 equiv.) in DMF/water (141 μL, 10:1). Purification by RP-HPLC afforded intermediate III.32 (20.39 mg, 38%) as yellowish solid. C₂₆₂H₄₁₂N₄₀O₁₁₆. HPLC (method X): t_{R}=10.4 min. MS (ESI): found m/z=1494.6 [M+4H]⁴⁺, calcd. m/z=1494.7 [M+4H]⁴⁺.

### Synthesis of IV.32

Compound IV.32 was synthesized according to GP4 using the TFA-salt of III.32 (20.39 mg, 3.4 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (1.95 mg, 3.8 μmol, 1.1 equiv.) and DIPEA (2.1 μL, 10.1 μmol, 3 equiv.) in DMF (0.82 mL). Purification by RP-HPLC afforded compound IV.32 (14.96 mg, 70%) as colorless solid. C₂₈₀H₄₃₈N₄₂O₁₂₄. HPLC (method Z): t_{R}=8.3 min. MS (ESI): found m/z=1594.6 [M+4H]⁴⁺, calcd. m/z=1594.2 [M+4H]⁴⁺.

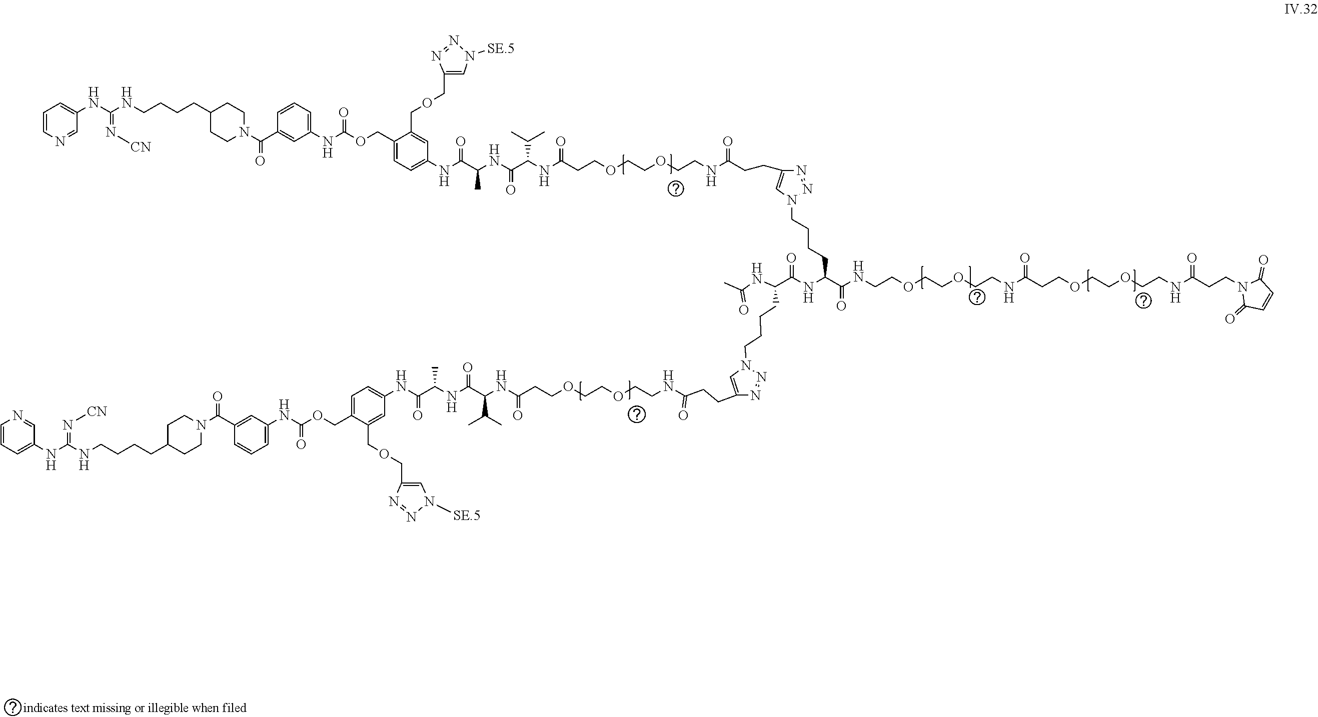

### Synthesis of I.33

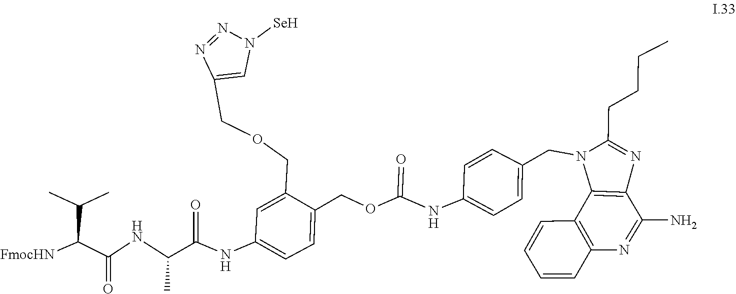

Intermediate I.33 was synthesized according to GP1 using intermediate 4.3 (60 mg, 37.9 μmol, 1 equiv.), intermediate 1.19 (44.3 mg, 41.7 μmol, 1.1 equiv.), copper (II) sulfate pentahydrate (1.9 mg, 7.6 μmol, 0.2 equiv.), tris(benzyltriazolylmethyl)amine (5.07 mg, 9.5 μmol, 0.25 equiv.) and sodium ascorbate (3.74 mg, 18.9 μmol, 0.5 equiv.) in DMF/water (660 μL, 10:1). Purification by RP-HPLC afforded 1.33 (95.5 mg, 91%) as colorless solid. C₁₁₇H₁₆₄N₁₂O₅₀. HPLC (method D): t_{R}=9.2 min. MS (ESI): found m/z=1269.8 [M+2H]²⁺, calcd. m/z=1269.5 [M+2H]²⁺.

### Synthesis of II.33

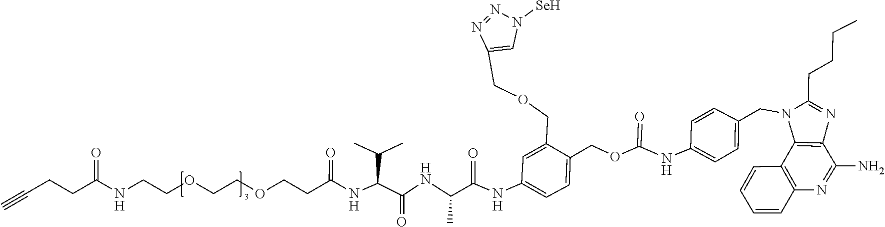

Intermediate II.33 was synthesized according to GP3 using 56 μL diethylamine (542 μmol, 24 equiv.) in 3 mL DMF for Fmoc-deprotection of intermediate 1.33 (60 mg, 22.6 μmol, 1 equiv.) and intermediate 3.2 (12.2 mg, 24.9 μmol, 1.1 equiv.), diisopropylethylamine (11.5 μL, 67.8 μmol, 3 equiv.) in DMF (1.5 mL) for the coupling reaction. Purification by RP-HPLC afforded intermediate II.33 (46.9 mg, 74%) as colorless solid. C₁₁₈H₁₇₉N₁₃O₅₄. HPLC (method AB): t_{R}=11.0 min. MS (ESI): found m/z=1322.6 [M+2H]²⁺, calcd. m/z=1322.1 [M+2H]²⁺.

### Synthesis of III.33

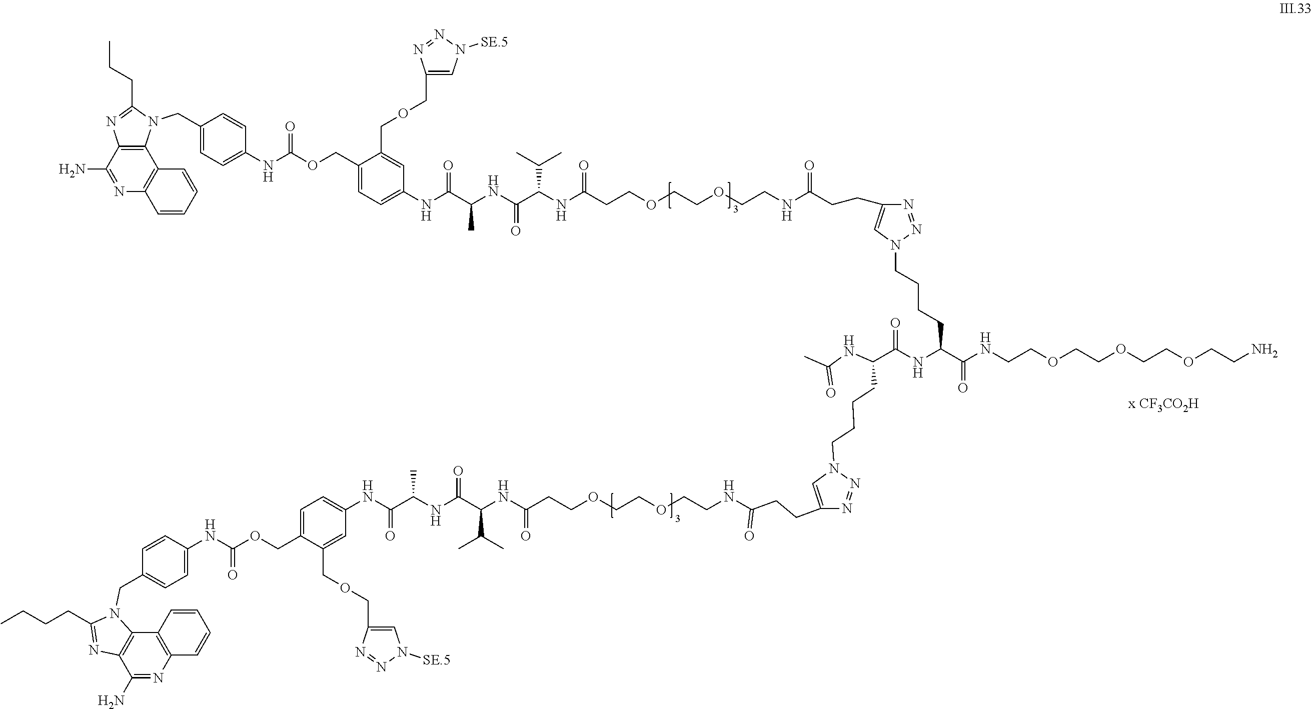

Intermediate III.33 was synthesized according to GP2 using intermediate 2.1 (3.62 mg, 6.2 μmol, 1 equiv.), intermediate II.33 (42.9 mg, 15.6 μmol, 2.5 equiv), copper (II) sulfate pentahydrate (1.95 mg, 7.8 μmol, 1.25 equiv.), Tris(3-hydroxypropyltriazolylmethyl)amine (6.78 mg, 15.6 μmol, 2.5 equiv.) and sodium ascorbate (4.64 mg, 23.4 μmol, 3.75 equiv.) in DMF/water (990 μL, 10:1). Purification by RP-HPLC afforded intermediate III.33 (19.7 mg, 50%) as colorless solid. C₂₅₈H₄₀₀N₃₆O₁₁₄. HPLC (method L): t_{R}=15.5 min. MS (ESI): found m/z=1458.7 [M+4H]⁴⁺, calcd. m/z=1457.7 [M+4H]⁴⁺.

### Synthesis of IV.33

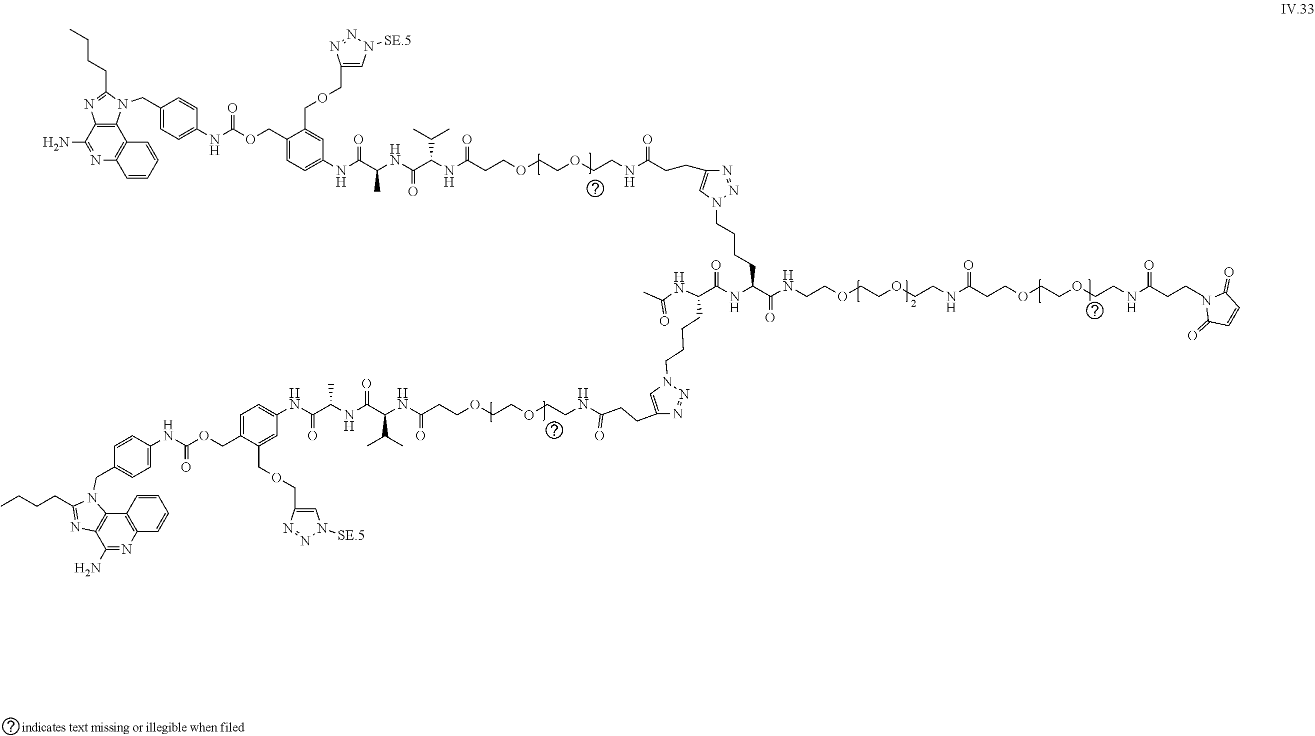

Compound IV.33 was synthesized according to GP4 using the TFA-salt of III.33 (19.7 mg, 3.1 μmol, 1 equiv.), N-[15-[(2,5-Dioxo-1-pyrrolidinyl)oxy]-15-oxo-3,6,9,12-tetraoxapentadec-1-yl]-2,5-dihydro-2,5-dioxo-1H-pyrrole-1-propanamide=Mal-PEG4-NHS-ester (1.77 mg, 3.4 μmol, 1.1 equiv.) and DIPEA (0.65 μL, 3.8 μmol, 1.2 equiv.) in DMF (0.79 mL). Purification by RP-HPLC afforded compound IV.33 (17.16 mg, 83%) as colorless solid. C₂₇₆H₄₂₆N₃₈O₁₂₂. HPLC (method L): t_{R}=16.1 min. MS (ESI): found m/z=1558.4 [M+4H]⁴⁺, calcd. m/z=1557.2 [M+4H]⁴⁺.

### Synthesis of IV.34

Compound IV.34 was synthesized as described for compound IV.33 but using intermediate 5.2. Compound IV.34 (6.34 mg, 65%) was obtained as colorless solid. C₂₁₈H₃₂₂N₅₈₀₅₈. HPLC (method AC): t_{R}=13.1 min. MS (ESI): found m/z=1172.6 [M+4H]⁴⁺, calcd. m/z=1171.1 [M+4H]⁴⁺.

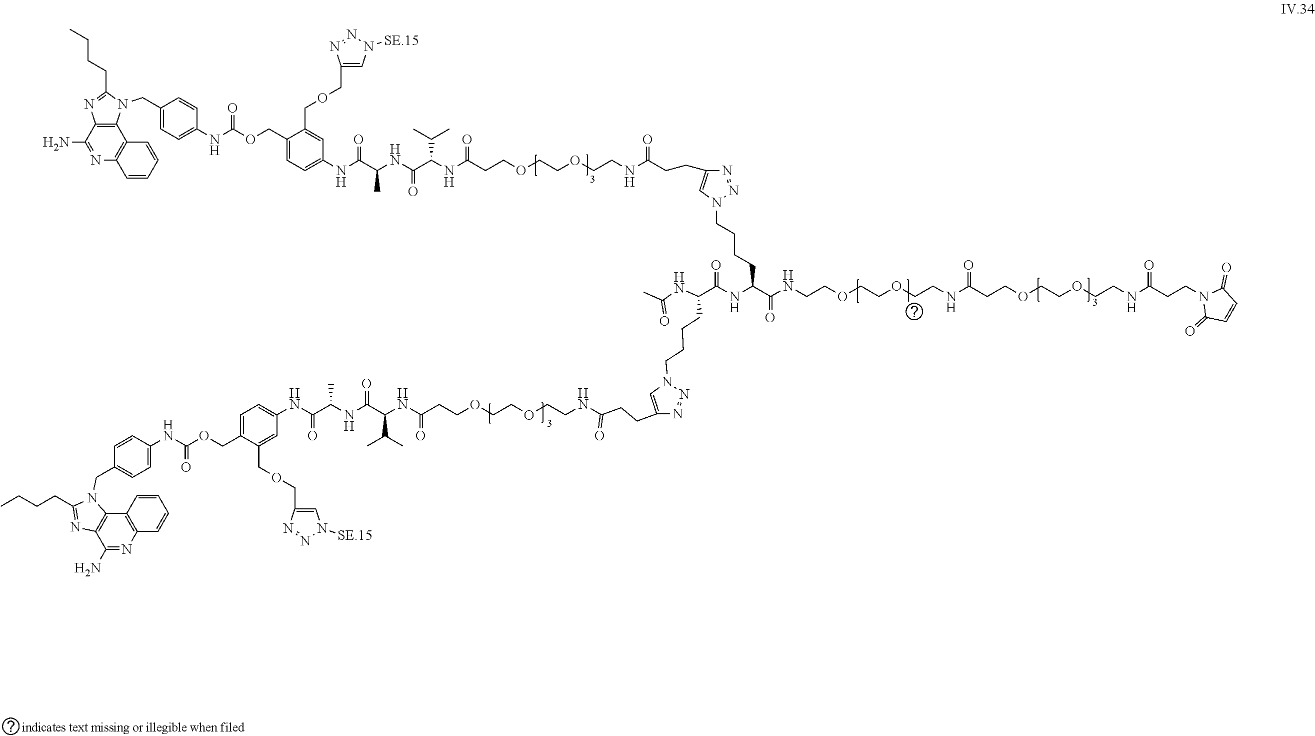

### Synthesis of I.35

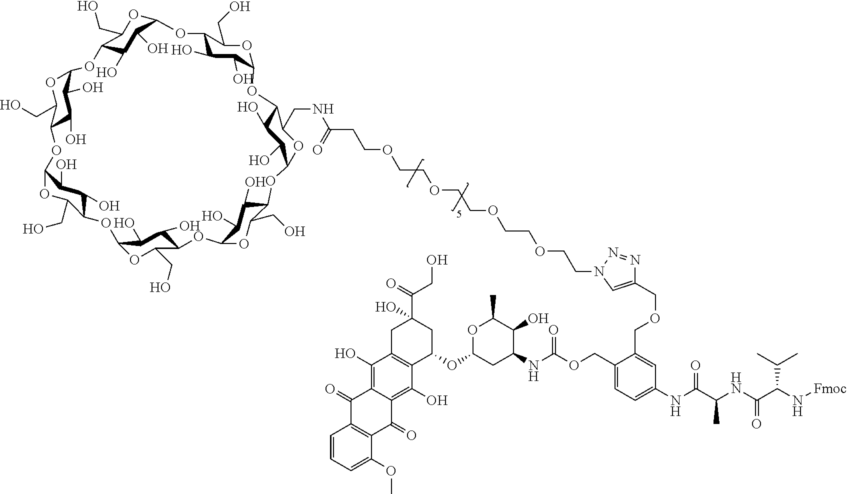

Intermediate I.35 was synthesized according to GP1 using Intermediate 4.3 (247 mg, 156 μmol, 1.2 equiv.), intermediate 1.17 (150 mg, 130 μmol, 1.0 equiv.) in DMF (1300 μL) with 325 μL aqueous catalyst solution containing 200 mM copper (II) sulfate pentahydrate, 240 mM tris(3-hydroxypropyltriazolylmethyl)amine and 600 mM sodium ascorbate. Purification by RP-HPLC afforded intermediate 1.35 as orange solid (306.8 mg, 90%) HPLC method A (480 nm): t_{R}=10.1 min. MS (ESI): found m/z=1368.5 [M+2H]²⁺, calcd. m/z=1369.0.

### Synthesis of II.35

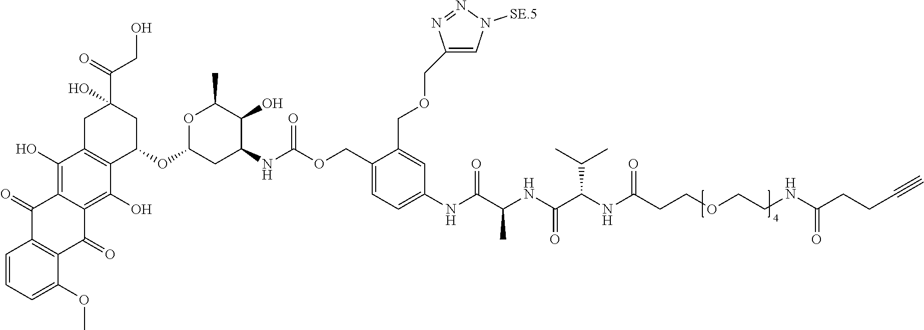

Intermediate II.35 was synthesized according to GP3 using intermediate 1.35 (218 mg, 79.7 μmol, 1 equiv.), 164 μL diethylamine (1.59 mmol, 20 equiv.) in 1.5 mL DMF for Fmoc-deprotection and intermediate 3.2 (49 mg, 99.6 μmol, 1.25 equiv.), diisopropylethylamine (41.7 μL, 239 μmol, 3 equiv.) in DMF (1600 μL) for the coupling reaction. Purification by RP-HPLC afforded intermediate II.35 (70.0 mg, 31%) as red solid. HPLC (method H): t_{R}=14.2 min. MS (ESI): found m/z=1421.5 [M+2H]²⁺, calcd. m/z=1421.58.

### Synthesis of III.35

Intermediate III.35 was synthesized according to GP2 using intermediate 2.1 (2.36 mg, 4.1 μmol, 1 equiv.), intermediate II.35 (27.8 mg, 9.78 μmol, 2.4 equiv.) in DMF (490 μL) with 49 μL aqueous catalyst solution containing 100 mM copper (II) sulfate, 120 mM tris(3-hydroxypropyltriazolylmethyl)amine and 300 mM sodium ascorbate. After 3 h the reaction was complete, and the reaction mixture was filtered through a 0.2 μm membrane filter and purified by prep. HPLC to yield Intermediate III.35 as a light red lyophilizate (17.4 mg, 64%). HPLC (method N, 480 nm): t_{R}=12.6 min. MS (ESI): found m/z=1556.8 [M+4H]⁴⁺, calcd. m/z=1557.4

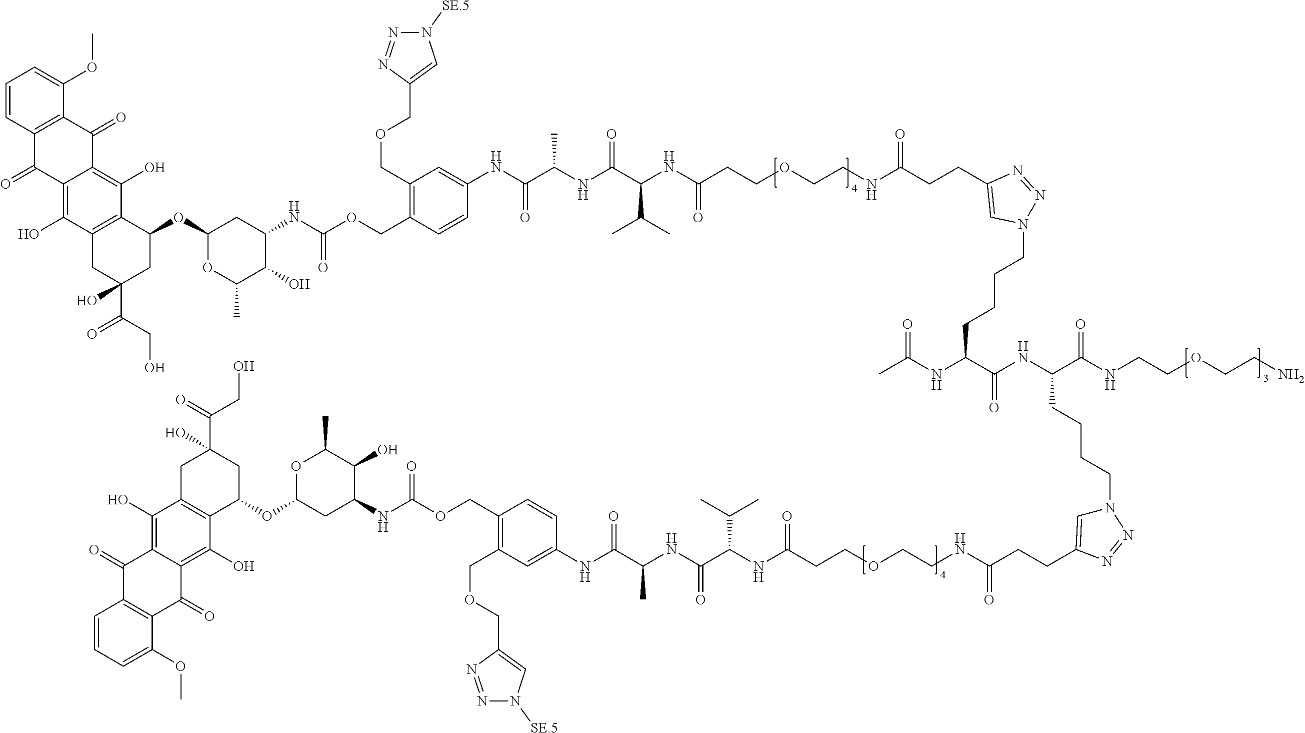

### Synthesis of IV.35

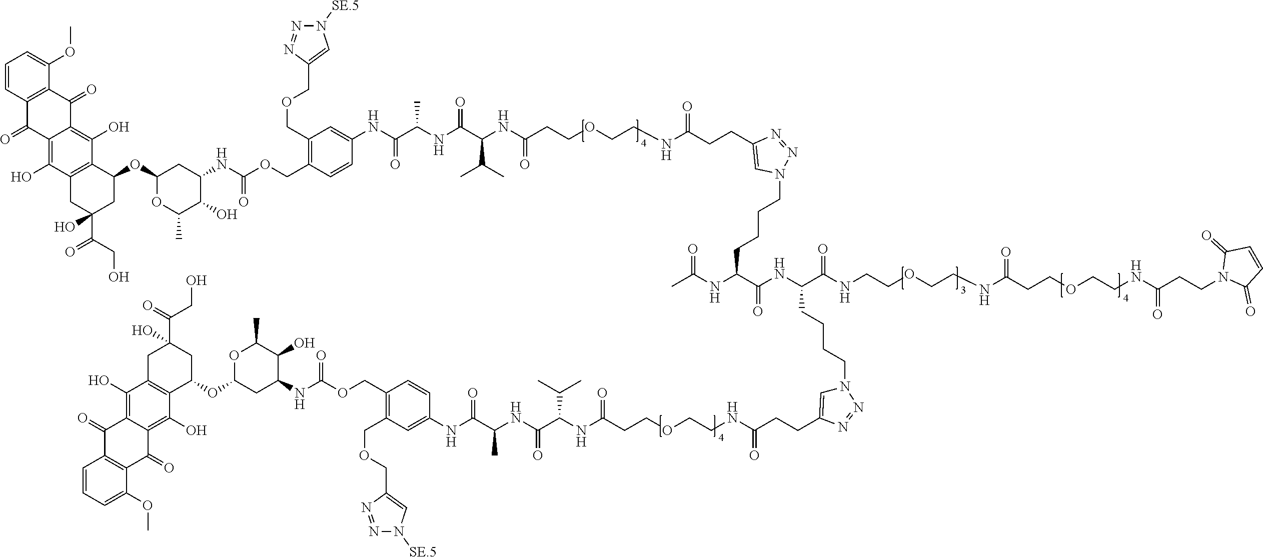

Compound IV.35 was synthesized according to GP4 using the TFA-salt of intermediate III.35 (17.4 mg, 2.79 μmol, 1 equiv.), Mal-PEG4-NHS-ester (5.75 mg, 11.2 μmol, 4 equiv.) and DIPEA (1.95 μL, 11.2 μmol, 4 equiv.) in DMF (560 μL). Upon completion after 0.5 h, the reaction mixture was acidified with TFA (1.7 μL), filtered through a 0.2 μm membrane filter (Nylon) and purified by RP-HPLC to afford compound IV.35 (15.83 mg, 86%) as light red solid. HPLC (method N): t_{R}=13.6 min. MS (ESI): found m/z=1656.9521 [M+4H]⁴⁺, calcd. m/z=1656.9566 [C₂₈₈H₄₄₂N₃₀O₁₄₄]⁴⁺.

### Synthesis of III.36

Intermediate III.36 was synthesized according to GP2 using intermediate 2.2 (2.94 mg, 3.45 μmol, 1 equiv.), intermediate II.35 (40.00 mg, 14.1 μmol, 4.1 equiv.), copper (II) sulfate pentahydrate (3.52 mg, 14.1 μmol, 4.1 equiv.), Tris(3-hydroxypropyltriazolylmethyl)amine (7.34 mg, 16.9 μmol, 4.9 equiv.) and sodium ascorbate (5.59 mg, 28.2 μmol, 8.2 equiv.) in DMF/water (1100 μL, 10:1). After 1.5 h the reaction was complete, and the reaction mixture was filtered through a 0.2 μm membrane filter and purified by prep. HPLC to yield Intermediate III.36 as a light red lyophilizate (27.9 mg, 66%). HPLC (method H, 480 nm): t_{R}=12.5 min.

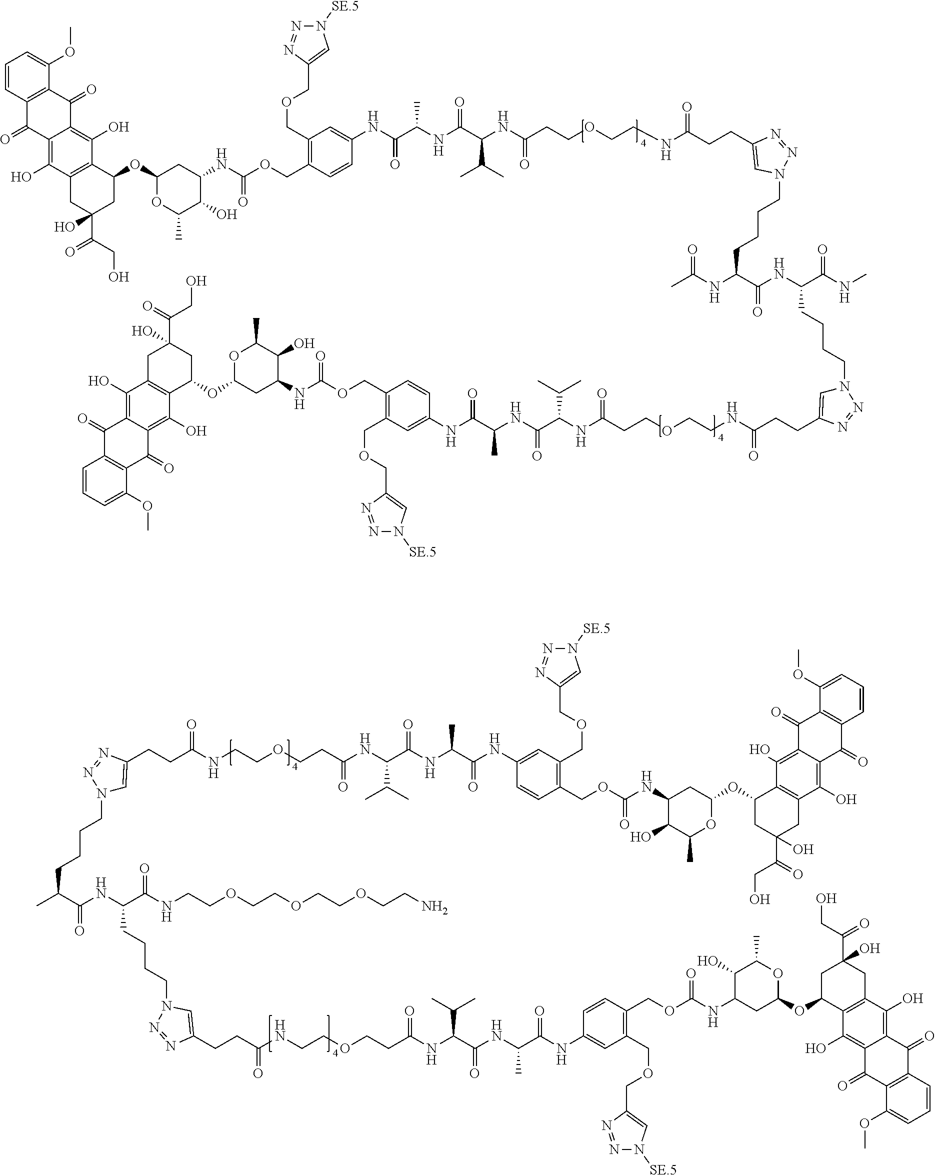

### Synthesis of IV.36

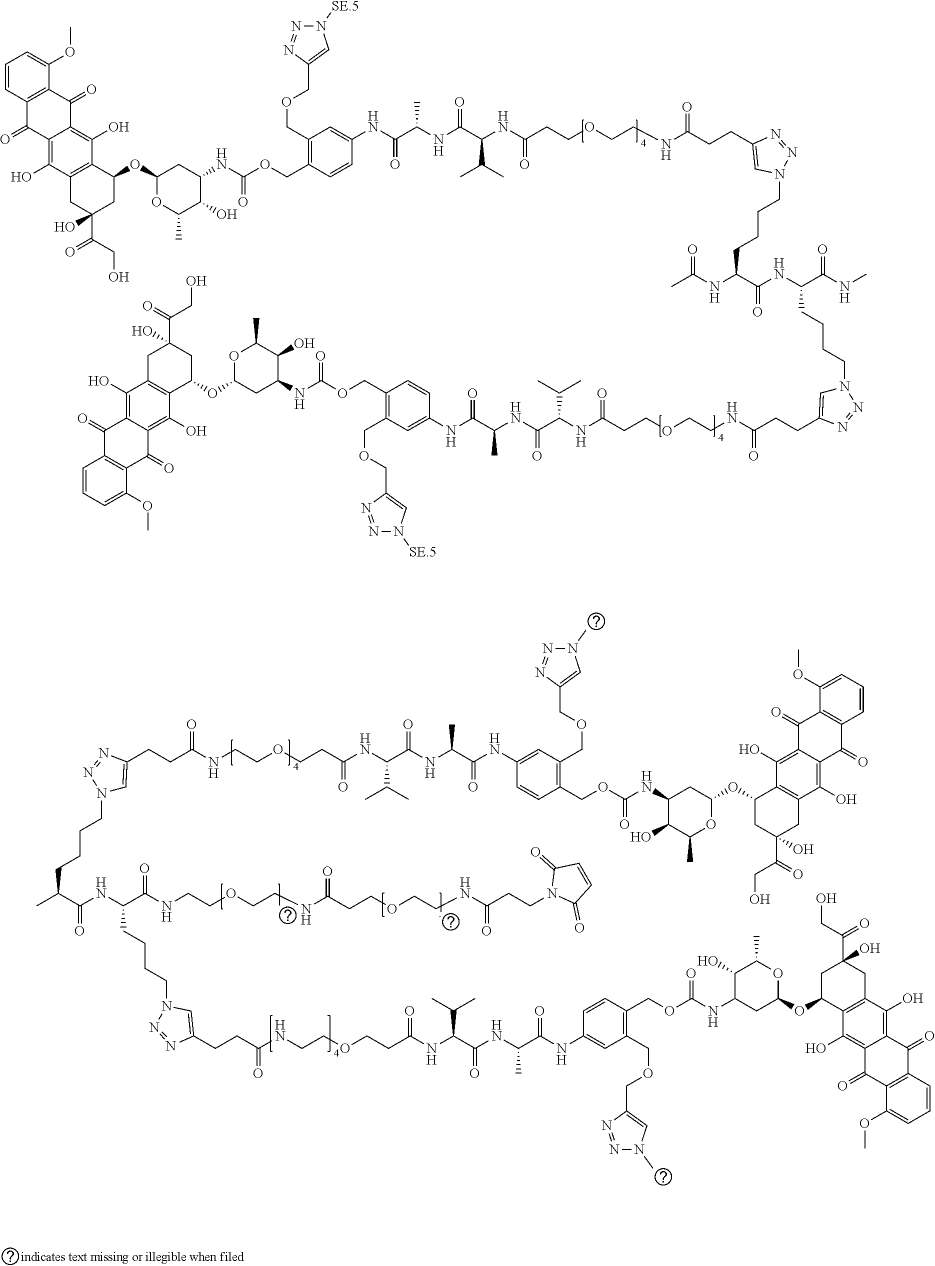

Compound IV.36 was synthesized according to GP4 using the TFA-salt of intermediate III.36 (10.0 mg, 0.82 μmol, 1 equiv.), Mal-PEG4-NHS-ester (1.68 mg, 3.27 μmol, 4 equiv.) and DIPEA (0.57 μL, 3.27 μmol, 4 equiv.) in DMF (160 μL). Upon completion after 0.5 h, the reaction mixture was acidified with TFA (1.0 μL), filtered through a 0.2 μm membrane filter (Nylon) and purified by RP-HPLC to afford compound IV.36 (9.62 mg, 93%) as light red solid. HPLC (method N): t_{R}=13.6 min. MS (ESI): found m/z=2103.7032 [M+6H]⁶⁺, calcd. m/z=2103.3847 [C₅₄₈H₈₃₄N₅₆O₂₇₆]⁶⁺.

### Synthesis of IV.37

Compound IV.37 was synthesized according to GP4 using the TFA-salt of intermediate III.36 (10.0 mg, 0.82 μmol, 1 equiv.), Mal-PEG₈-NHS-ester (2.26 mg, 3.27 μmol, 4 equiv.) and DIPEA (0.57 μL, 3.27 μmol, 4 equiv.) in DMF (160 μL). Upon completion after 45 min, the reaction mixture was acidified with TFA (1.0 μL), filtered through a 0.2 μm membrane filter (Nylon) and purified by RP-HPLC to afford compound IV.36 (8.1 mg, 77%) as light red solid. HPLC (method N): t_{R}=12.9 min. MS (ESI): found m/z=2133.3941 [M+6H]⁶⁺, calcd. m/z=2132.7355 [C₅₆H₈₅₀N₅₆O₂₈₀]⁶⁺.

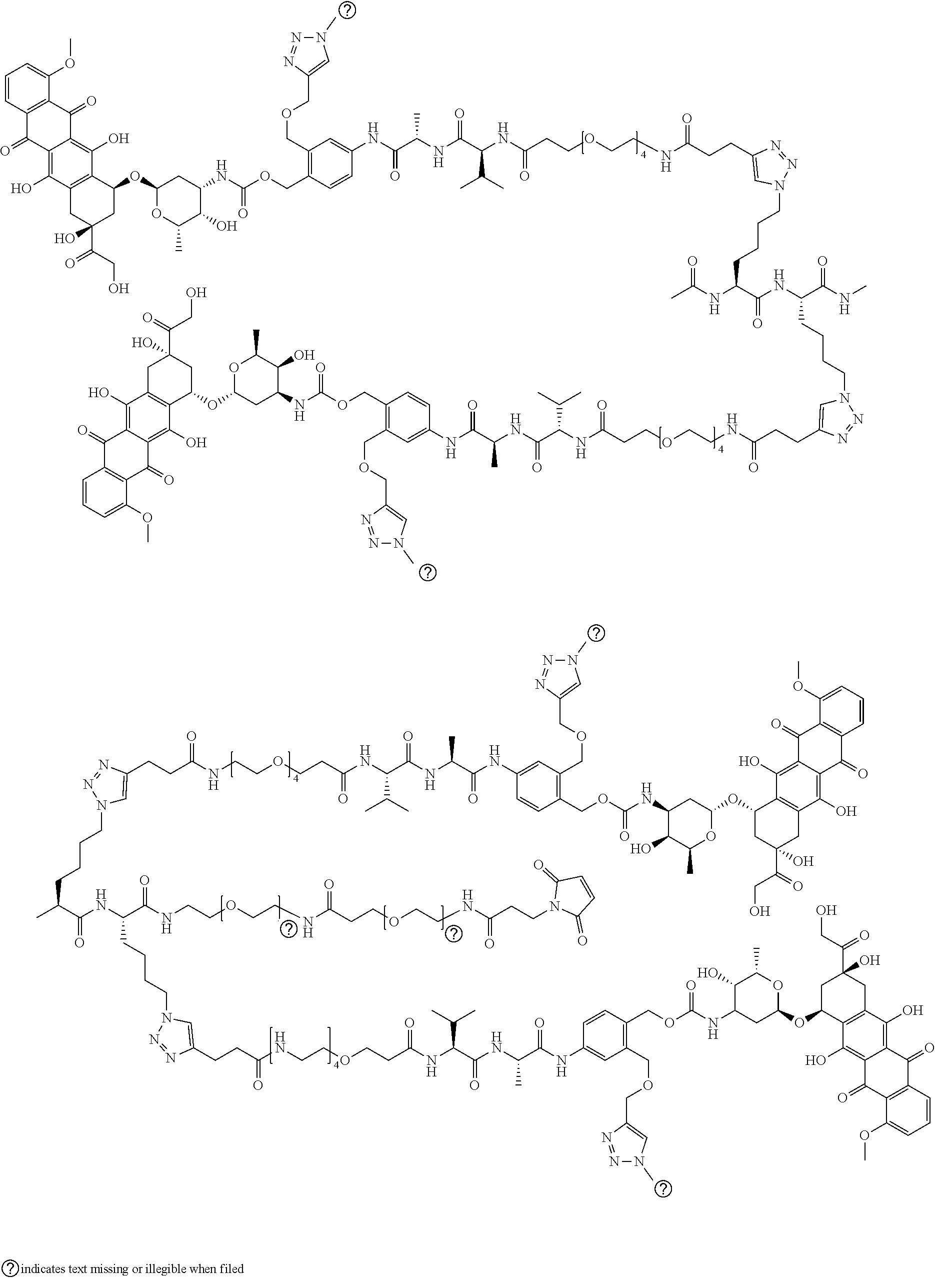

### Synthesis of I.38

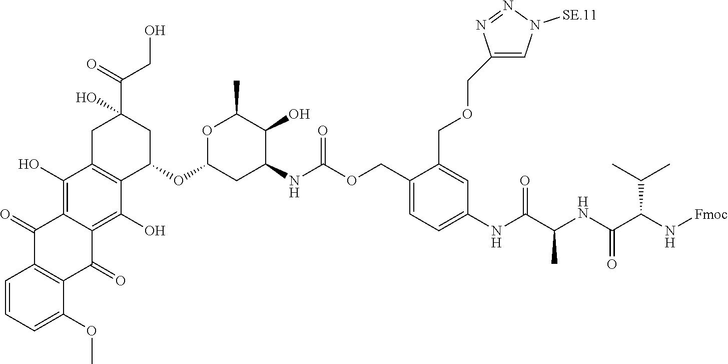

Intermediate I.38 was synthesized according to GP1 using Intermediate 4.9 (234 mg, 156 μmol, 1.2 equiv.), intermediate 1.17 (150 mg, 130 μmol, 1.0 equiv.) in DMF (1300 μL) with 325 μL aqueous catalyst solution containing 200 mM copper (II) sulfate pentahydrate, 240 mM tris(3-hydroxypropyltriazolylmethyl)amine and 600 mM sodium ascorbate. Purification by RP-HPLC afforded intermediate 1.38 as orange solid (252.9 mg, 74%) HPLC method A (480 nm): t_{R}=9.8 min.

### Synthesis of II.38

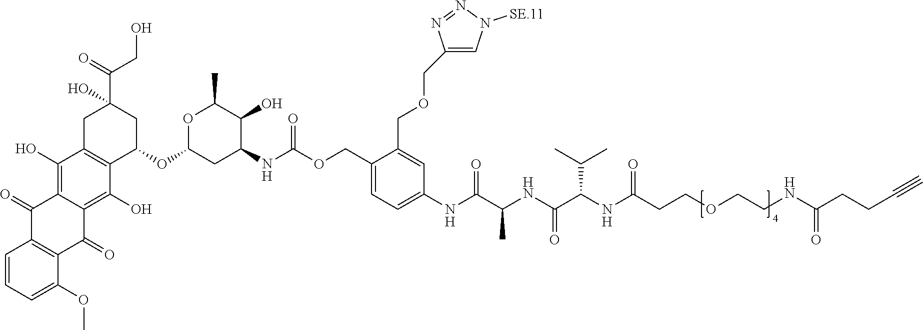

Intermediate II.38 was synthesized according to GP3 using intermediate 1.38 (252 mg, 95.4 μmol, 1 equiv.), 197 μL diethylamine (1.91 mmol, 20 equiv.) in 2.5 mL DMF for Fmoc-deprotection. After 30 min the crude was purified by prep. HPLC to result in 39 mg (17%) of the free amine, HPLC (method A): t_{R}=7.4 min. MS (ESI): found m/z=1215.5 [M+2H]²⁺, calcd. m/z=1215.5. The free amine was subsequently reacted with intermediate 3.2 (9.86 mg, 20.1 μmol, 1.25 equiv.), diisopropylethylamine (8.41 μL, 48.3 μmol, 3 equiv.) in DMF (390 μL) for the coupling reaction. Purification by RP-HPLC afforded intermediate II.38 (34.7 mg, 78%) as red solid. HPLC (method A): t_{R}=8.4 min. MS (ESI): found m/z=1379.7 [M+2H]²⁺, calcd. m/z=1379.0.

### Synthesis of III.38

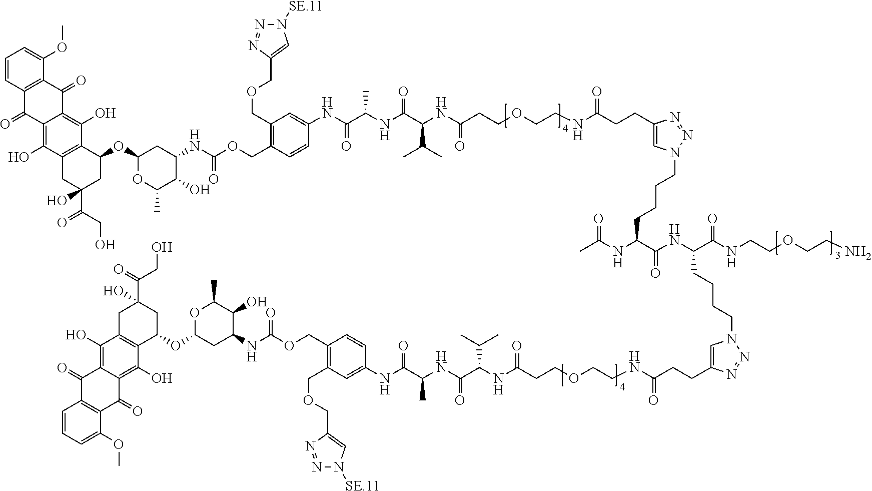

Intermediate III.38 was synthesized according to GP2 using intermediate 2.1 (3.3 mg, 5.7 μmol, 1 equiv.), intermediate II.38 (34.5 mg, 12.5 μmol, 2.4 equiv.) in DMF 5 (680 μL) with 68 μL aqueous catalyst solution containing 100 mM copper (II) sulfate pentahydrate, 120 mM tris(3-hydroxypropyltriazolylmethyl)amine and 300 mM sodium ascorbate. After 1 h the reaction was complete, and the reaction mixture was acidified with 1.95 μL TFA, filtered through a 0.2 μm membrane filter and purified by prep. HPLC to yield Intermediate III.38 as a light red lyophilizate (24.7 mg, 69%). HPLC (method N, 480 nm): t_{R}=11.05 min. MS (ESI): found m/z=1514.9 [M+4H]⁴⁺, calcd. m/z=1514.9.

### Synthesis of IV.38

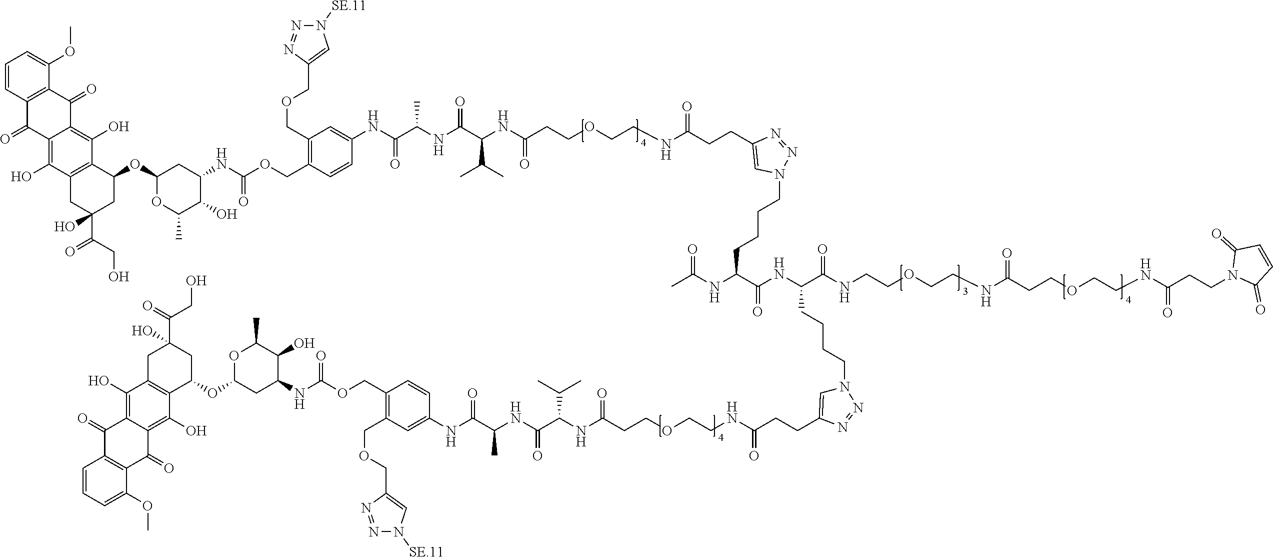

Compound IV.38 was synthesized according to GP4 using the TFA-salt of intermediate III.38 (12.0 mg, 1.98 μmol, 1 equiv.), Mal-PEG4-NHS-ester (4.07 mg, 7.92 μmol, 4 equiv.) and DIPEA (1.37 μL, 11.2 μmol, 4 equiv.) in DMF (560 μL). Upon completion after 0.5 h, the reaction mixture was acidified with TFA (1.7 L), filtered through a 0.2 μm membrane filter (Nylon) and purified by RP-HPLC to afford compound IV.38 (8.23 mg, 65%) as light red solid. HPLC (method N): t_{R}=12.5 min. MS (ESI): found m/z=1614.3937 [M+4H]⁴⁺, calcd. m/z=1614.1612 [C₂₇₈H₄₂₀N₂₈O₁₄]⁴⁺.

### Synthesis of I.39

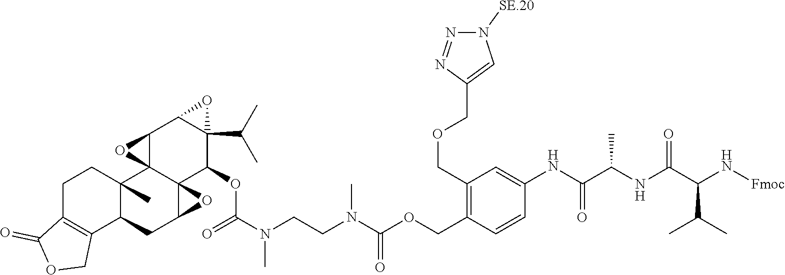

Intermediate I.39 was synthesized according to GP1 using Intermediate 4.9 (247 mg, 156 μmol, 1.2 equiv.), intermediate 1.21 (150 mg, 130 μmol, 1.0 equiv.) in DMF (672 μL) with 336 μL aqueous catalyst solution containing 100 mM copper (II) sulfate pentahydrate, 120 mM tris(3-hydroxypropyltriazolylmethyl)amine and 300 mM sodium ascorbate. Purification by RP-HPLC afforded intermediate 1.39 as colorless solid (140.19 mg, 81%) HPLC method A (260 nm): t_{R}=10.3 min. MS (ESI): found m/z=1292.1 [M+2H]²⁺, calcd. m/z=1292.0.

### Synthesis of II.39

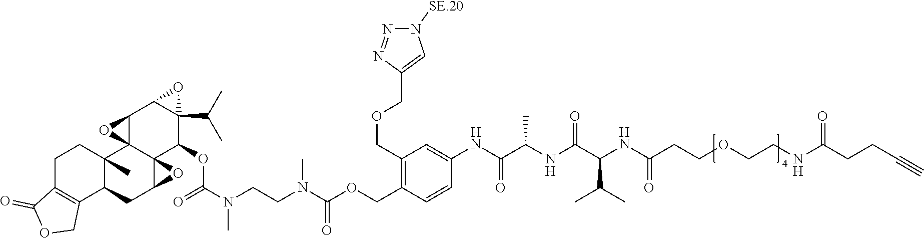

Intermediate II.39 was synthesized according to GP3 using intermediate 1.39 (140.19 mg, 54.28 μmol, 1 equiv.), 281 μL diethylamine (2.71 mmol, 50 equiv.) in 1.6 mL DMF for Fmoc-deprotection to the free amine, HPLC (method A): t_{R}=7.2 min. MS (ESI): found m/z=1181.4 [M+2H]²⁺, calcd. m/z=1180.98. The crude amine was subsequently reacted with intermediate 3.2 (34.61 mg, 70.57 μmol, 1.3 equiv.) and diisopropylethylamine (24.0 μL, 141 μmol, 2.6 equiv.) in DMF (1000 μL) for the coupling reaction. Purification by RP-HPLC afforded intermediate II.39 (103.77 mg, 71%) as colorless solid. HPLC (method H): t_{R}=13.5 min. MS (ESI): found m/z=1344.8 [M+2H]²⁺, calcd. m/z=1344.6.

### Synthesis of III.39

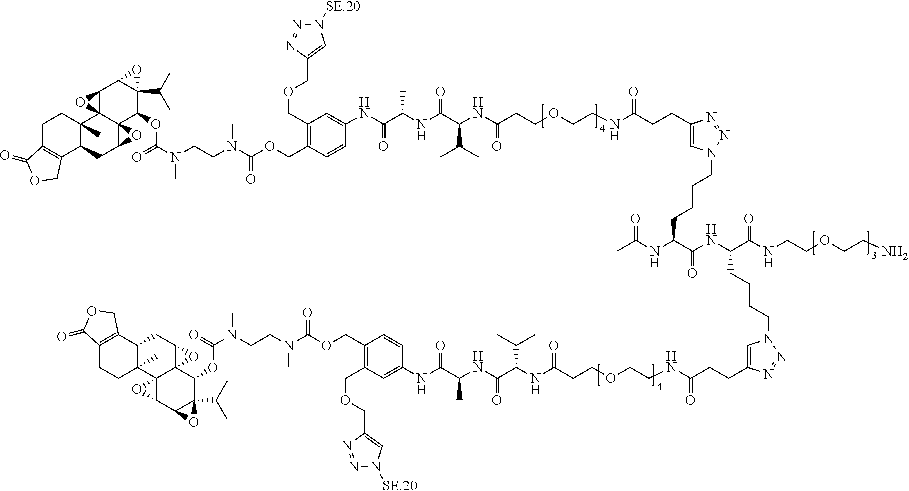

Intermediate III.39 was synthesized according to GP2 using intermediate 2.1 (2.41 mg, 4.17 μmol, 1 equiv.), intermediate II.30 (26.88 mg, 10 μmol, 2.4 equiv.) in DMF (500 μL) with 50 μL aqueous catalyst solution containing 100 mM copper (II) sulfate pentahydrate, 120 mM tris(3-hydroxypropyltriazolylmethyl)amine and 300 mM sodium ascorbate. After 50 min the reaction was complete, and the reaction mixture was filtered through a 0.2 μm membrane filter and purified by prep. HPLC to yield Intermediate III.39 as a colorless lyophilizate (16.60 mg, 53%). HPLC (method N, 250 nm): t_{R}=12.7 min. MS (ESI): found m/z=1973.7; [M+4H]⁴⁺, calcd. m/z=1973.53.

### Synthesis of IV.39

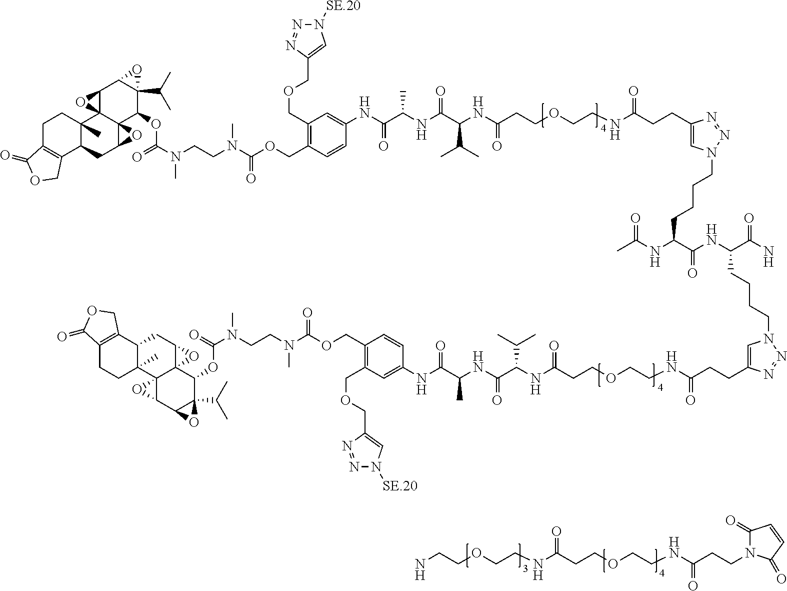

Compound IV.39 was synthesized according to GP4 using the TFA-salt of intermediate III.39 (16.06 mg, 2.66 μmol, 1 equiv.), Mal-PEG4-NHS-ester (2.73 mg, 5.32 μmol, 2 equiv.) and DIPEA (0.90 μL, 5.32 μmol, 2 equiv.) in DMF (266 μL). Upon completion after 2.5 h, the reaction mixture was filtered through a 0.2 μm membrane filter (Nylon) and purified by RP-HPLC to afford compound IV.39 (13.78 mg, 82%) as colorless lyophilizate. HPLC (method J, 250 nm): t_{R}=12.5 min. MS (ESI): found m/z=1580.1 [M+4H]⁴⁺, calcd. m/z=1579.69 [C₂₇₄H₄₃₀N₃₀O₁₃₆]⁴⁺.

### Synthesis of III.40

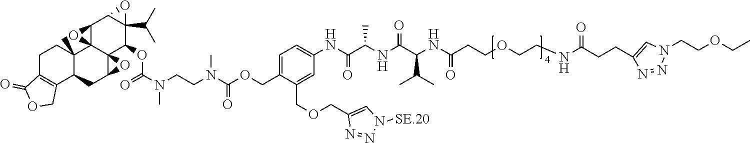

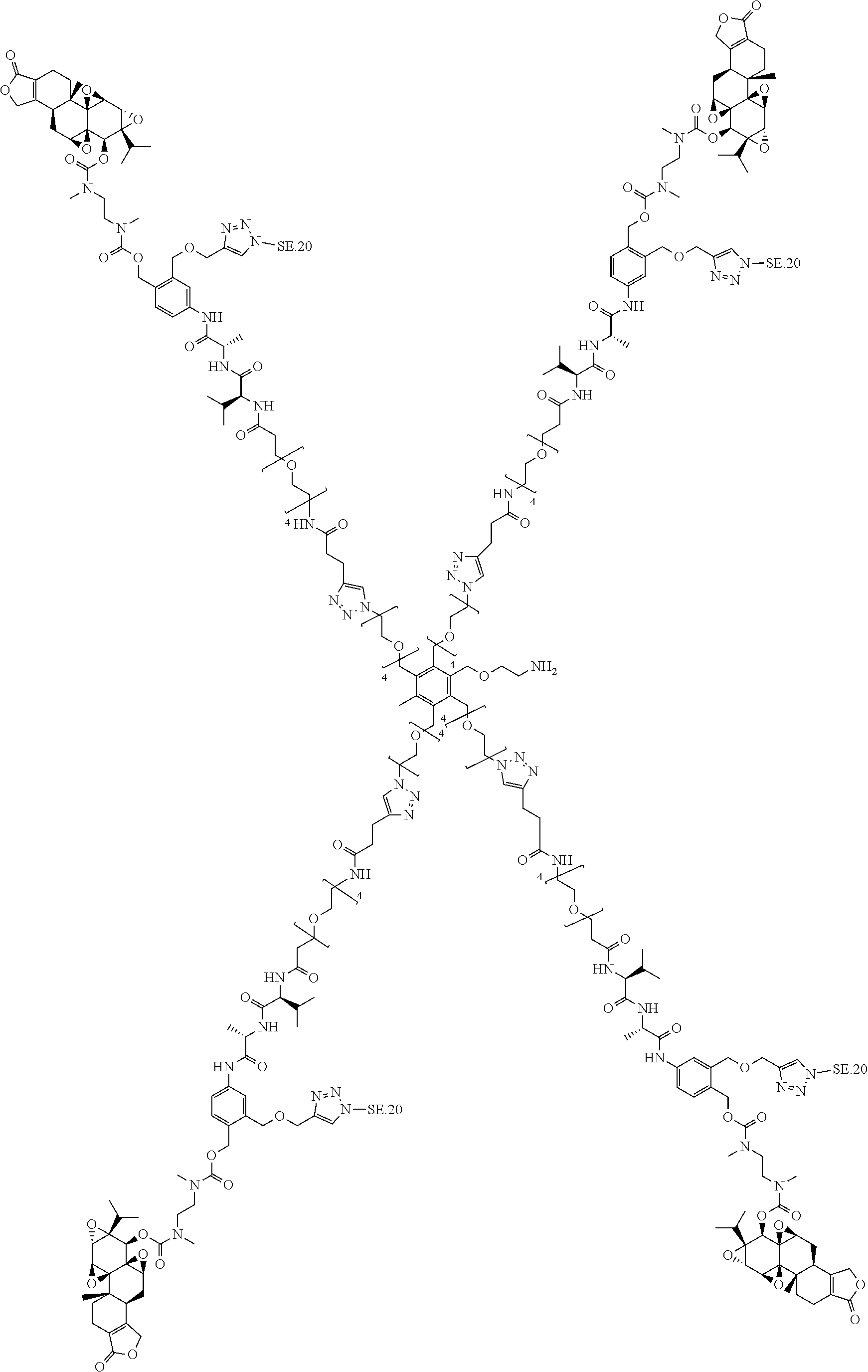

Intermediate III.40 was synthesized according to GP2 using intermediate 2.4 (5.18 mg, 3.33 μmol, 1 equiv.), intermediate II.39 (53.75 mg, 20.00 μmol, 6.0 equiv.), in DMF (1000 μL) with 100 μL aqueous catalyst solution containing 100 mM copper (II) sulfate pentahydrate, 120 mM tris(3-hydroxypropyltriazolylmethyl)amine and 300 mM sodium ascorbate. After 50 min the reaction was complete, and the reaction mixture was filtered through a 0.2 μm membrane filter and purified by prep. HPLC to yield Intermediate III.40 as a colorless lyophilizate (36.27 mg, 73%). HPLC (method N, 250 nm): t_{R}=15.2 min. MS (ESI): found m/z=2480.73; [M+6H]⁶⁺, calcd. m/z=2479.74.

### Synthesis of IV.40

Compound IV.40 was synthesized according to GP4 using the TFA-salt of intermediate III.40 (36.27 mg, 2.42 μmol, 1 equiv.), Mal-PEG12-NHS-ester solution, 100 mM in DMF (48.4 μL, 4.84 μmol, 2 equiv.) and DIPEA ester solution, 100 mM in DMF (48.4 μL, 4.84 μmol, 2 equiv.) in DMF (242 μL). Upon completion after 4.5 h, the reaction mixture added dropwise to 10 mL of ice-cooled MTBE and the precipitate was collected by centrifugation (4000×g, 4 min) and washed with 10 mL fresh MTBE. The crude was dissolved in 0.05% TFA/ACN 80:20, filtered through a 0.2 μm membrane filter (Nylon) and purified by RP-HPLC to afford compound IV.40 (32.75 mg, 87%) as colorless lyophilizate. HPLC (method AE): t_{R}=15.4 min. MS (ESI): found m/z=2608.65 [M+6H]⁶⁺, calcd. m/z=2604.80 [C₆₇₉H₁₀₆₉N₆₃O₃₄₅]⁶⁺.

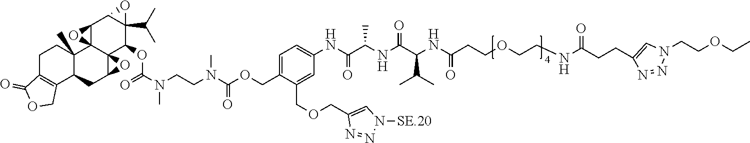

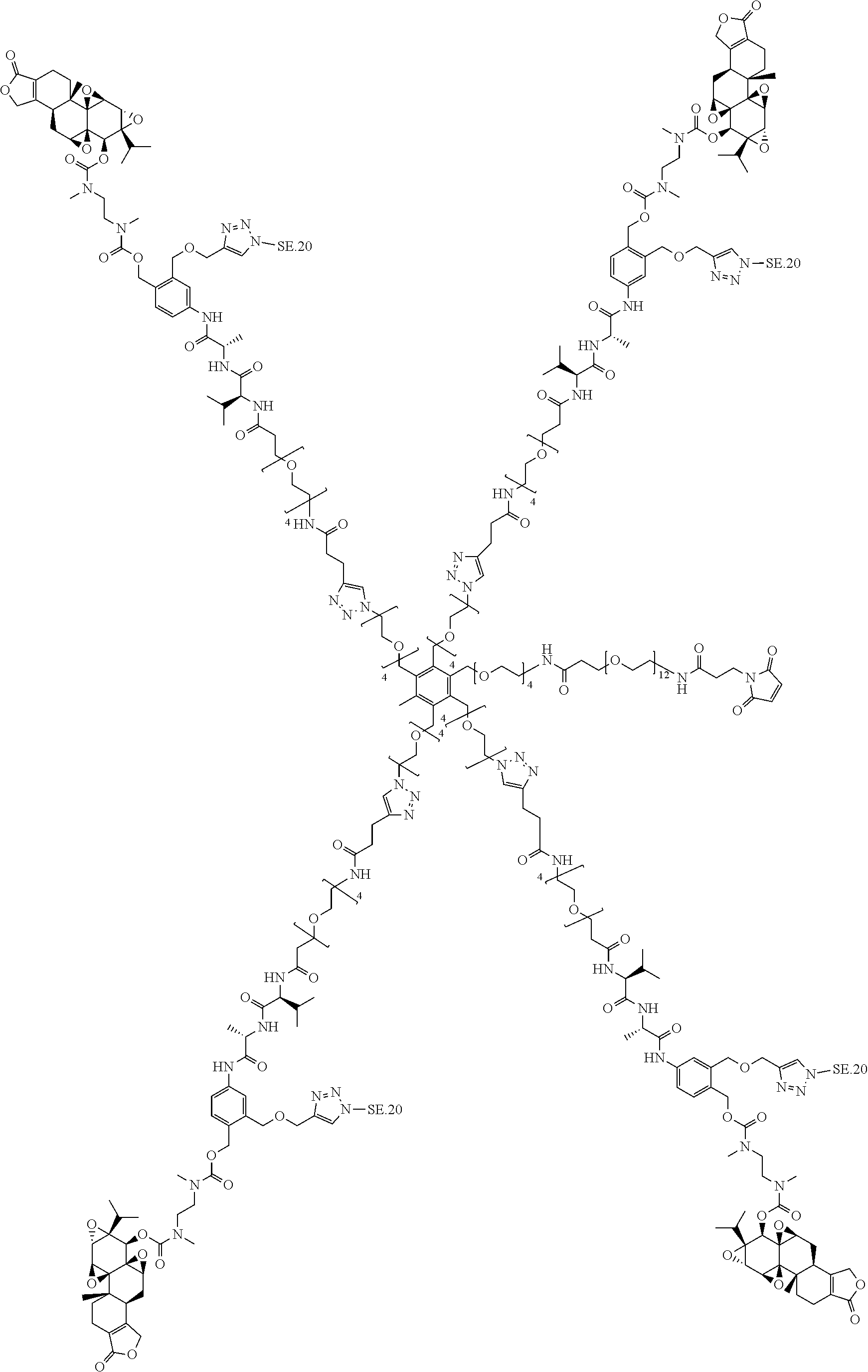

### Synthesis of I.41

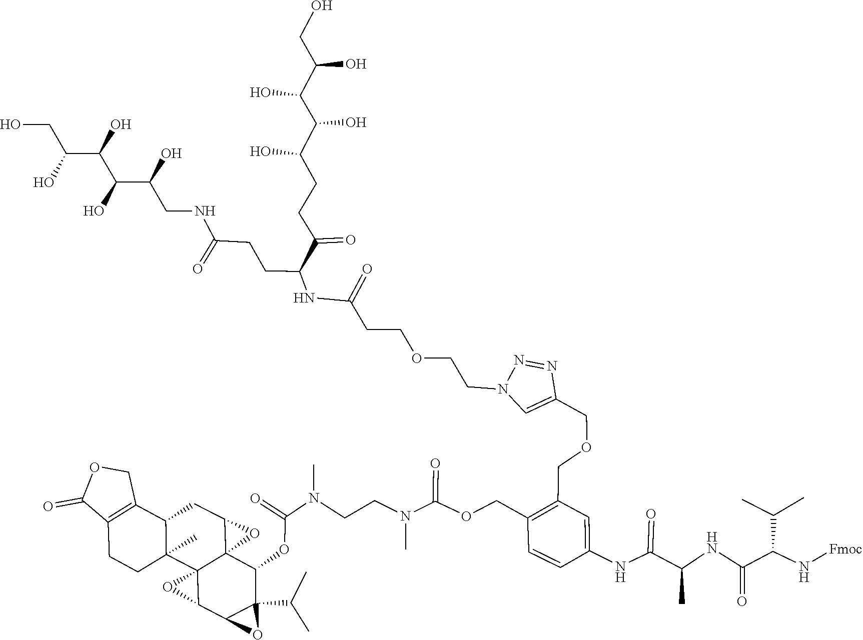

Intermediate I.41 was synthesized according to GP1 by stirring Intermediate 6.4 (72.97 mg, 79.06 μmol, 1.2 equiv.) and intermediate 1.21 (71.43 mg, 65.88 μmol, 1.0 equiv.) in DMF (659 μL) with 329 μL aqueous catalyst solution containing 100 mM copper (II) sulfate pentahydrate, 120 mM tris(3-hydroxypropyltriazolylmethyl)amine and 300 mM sodium ascorbate for 50 min. Purification by RP-HPLC afforded intermediate 1.41 as colorless solid (100.71 mg, 76%) HPLC method A (260 nm): t_{R}=10.9 min. MS (ESI): found m/z=1004.5 [M+2H]²⁺, calcd. m/z=1004.5 [C₉₆H₁₄₁N₁₁O₃₅]²⁺.

### Synthesis of II.41

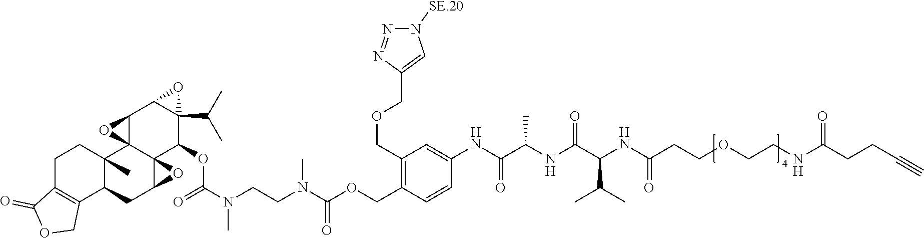

Intermediate II.41 was synthesized according to GP3 using intermediate 1.41 (140.19 mg, 54.28 μmol, 1 equiv.), 281 μL diethylamine (2.71 mmol, 50 equiv.) in 1.6 mL DMF for Fmoc-deprotection to the free amine, HPLC (method A): t_{R}=7.9 min. MS (ESI): found m/z=893.4 [M+2H]²⁺, calcd. m/z=892.9. The crude amine was subsequently reacted with intermediate 3.2 (31.99 mg, 65.23 μmol, 1.3 equiv.) and diisopropylethylamine (22.2 μL, 130.45 μmol, 2.6 equiv.) in DMF (1000 μL) for the coupling reaction. Purification by RP-HPLC afforded intermediate II.41 (71.32 mg, 67%) as colorless solid. HPLC (method H): t_{R}=13.5 min. MS (ESI): found m/z=1057.0 [M+2H]²⁺, calcd. m/z=1057.0 [C₉₇H₁₅₆N₁₂O₃₉]²⁺.

### Synthesis of III.41

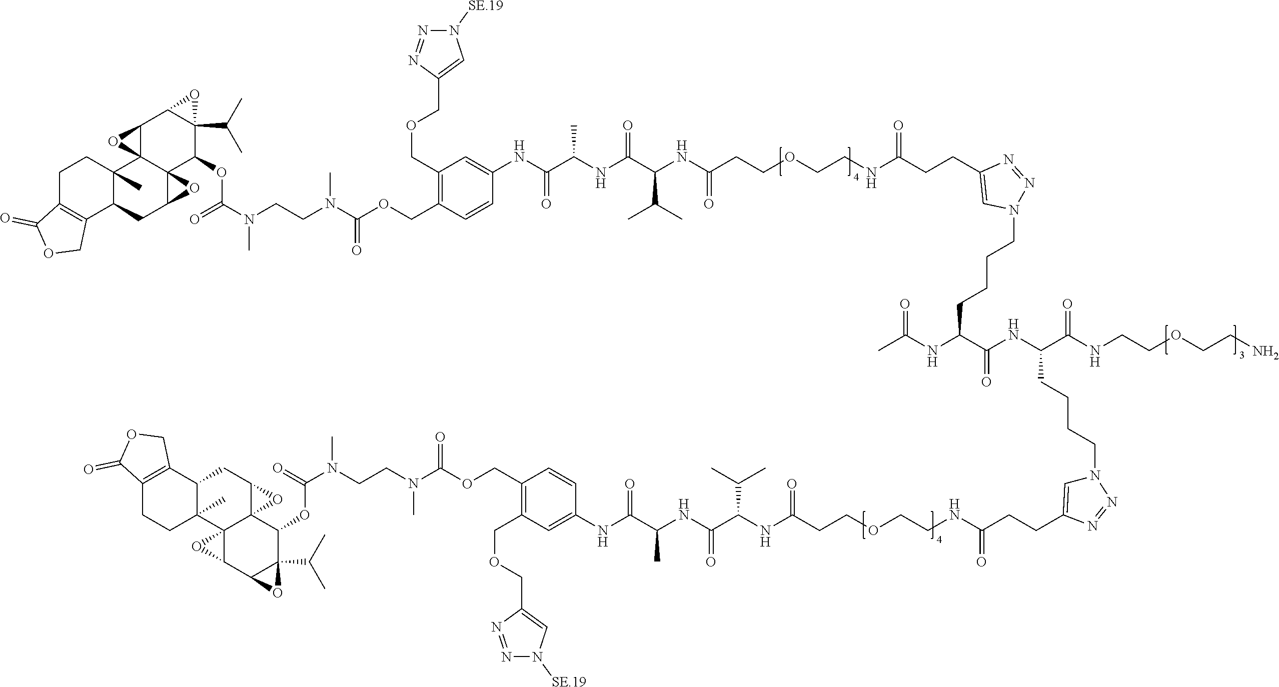

Intermediate III.41 was synthesized according to GP2 using intermediate 2.1 (2.41 mg, 4.17 μmol, 1 equiv.), intermediate II.41 (21.12 mg, 10.00 μmol, 2.4 equiv.) in DMF (500 μL) with 50 μL aqueous catalyst solution containing 100 mM copper (II) sulfate pentahydrate, 120 mM tris(3-hydroxypropyltriazolylmethyl)amine and 300 mM sodium ascorbate. After 50 min the reaction was complete, and the reaction mixture was acidified with 43.9 μL 5proz. TFA, filtered through a 0.2 μm membrane filter and purified by prep. HPLC to yield Intermediate III.41 as a colorless lyophilizate (14.30 mg, 59%). HPLC (method N, 250 nm): t_{R}=15.9 min. MS (ESI): found m/z=954.5 [M+5H]⁵⁺, calcd. m/z=954.3 [C₂₁₆H₃₅₅N₃₄O₈₄]⁵⁺.

### Synthesis of IV.41

Compound IV.41 was synthesized according to GP4 using the TFA-salt of intermediate III.41 (14.06 mg, 2.88 μmol, 1 equiv.), Mal-PEG4-NHS-ester solution, 100 mM in DMF (57.6 μL, 5.76 μmol, 2 equiv.) and DIPEA solution, 100 mM in DMF (57.6 μL, 5.76 μmol, 2 equiv.) in DMF (288 μL). Upon completion after 3 h, the reaction mixture was filtered through a 0.2 μm membrane filter (Nylon) and purified by RP-HPLC to afford compound IV.41 (12.52 mg, 84%) as colorless lyophilizate. HPLC (method J, 250 nm): t_{R}=13.8 min. MS (ESI): found m/z=1292.3 [M+4H]⁴⁺, calcd. m/z=1292.2 [C₂₃₄H₃₈₀N₃₆O₉₂]⁴⁺.

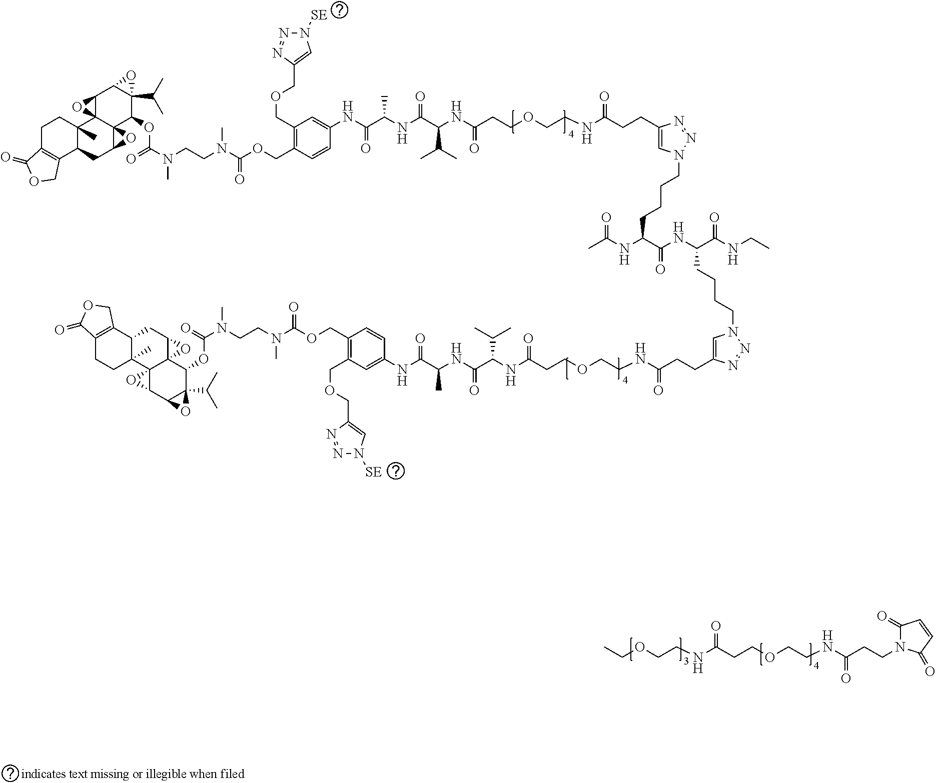

### Synthesis of III.42

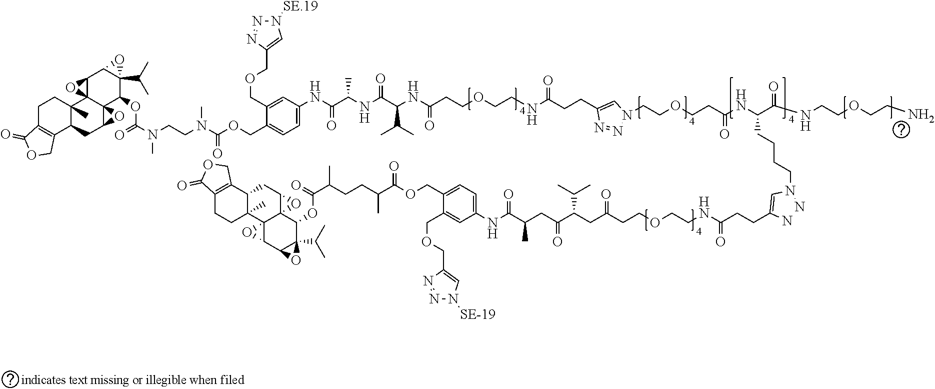

Intermediate III.42 was synthesized according to GP2 using intermediate 2.3 (3.99 mg, 3.33 μmol, 1 equiv.), intermediate II.41 (42.25 mg, 20.00 μmol, 6.0 equiv.) in DMF (1000 μL) with 100 μL aqueous catalyst solution containing 100 mM copper (II) sulfate pentahydrate, 120 mM tris(3-hydroxypropyltriazolylmethyl)amine and 300 mM sodium ascorbate. After 50 min the reaction was complete, and the reaction mixture was, filtered through a 0.2 μm membrane filter and purified by prep. HPLC to yield intermediate III.42 as a colorless lyophilizate (23.57 mg, 60%). HPLC (method N 250 nm): t_{R}=18.4 min. MS (ESI): found m/z=1941.3 [M+6H]⁶⁺, calcd. m/z=1941.0.

### Synthesis of IV.42

Compound IV.42 was synthesized according to GP4 using the TFA-salt of intermediate III.42 (23.57 mg, 2.00 μmol, 1 equiv.), Mal-PEG12-NHS-ester solution, 100 mM in DMF (40.1 μL, 4.01 μmol, 2 equiv.) and DIPEA solution, 100 mM in DMF (40.1 μL, 4.01 μmol, 2 equiv.) in DMF (200 μL). Upon completion after 4.5 h, the reaction mixture added dropwise to 10 ml of ice-cooled MTBE and the precipitate was collected by centrifugation (4000×g, 4 min) and washed with 10 mL fresh MTBE. The crude was dissolved in 0.05% TFA/ACN 80:20, filtered through a 0.2 μm membrane filter (Nylon) and purified by RP-HPLC to afford compound IV.42 (20.98 mg, 84%) as colorless lyophilizate. HPLC (method AE): t_{R}=18.4 min. MS (ESI): found m/z=1771.8 [M+7H]⁷⁺, calcd. m/z=1771.04 [C₅₆₂H₉₁₄N₈₃O₂₂₃]⁷⁺.

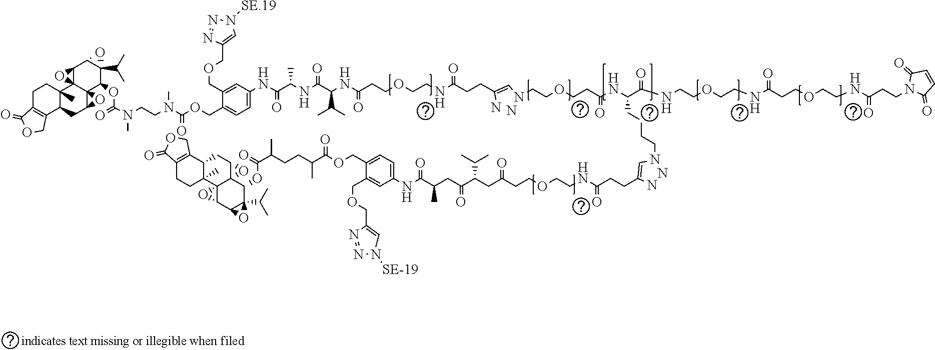

### Synthesis of II.43

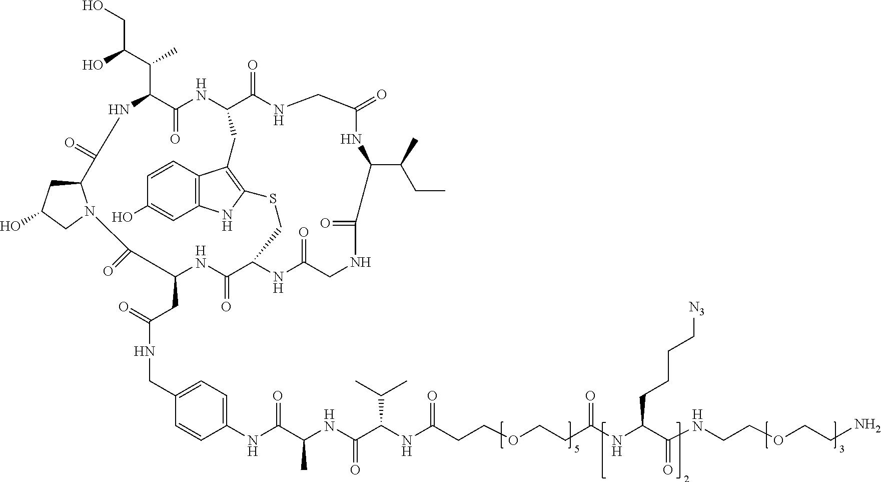

200 mg (approx. 40 μmol, 4 equiv.) resin loaded intermediate 2.5 was swollen for 10 min in abs. DCM (1 mL). filtered and a Fmoc-deprotection solution (1 mL) of 20% piperidine in DMF added to the resin. The suspension was incubated for 20 min, filtered and the deprotection procedure repeated once. Then, the resin was washed with DMF (3×1 min) and a solution of Bis-PEG 5-NHS ester (CAS: 756526-03-1, 85.2 mg, 160 μmol, 16 equiv.) and DIPEA (27.2 μL, 160 μmol, 16 equiv.) in 1 mL DMF was added followed by shaking overnight. The resin was then washed with 5×DMF, 3×DCM and 3×DMF. Next, intermediate 1.22 (12.92 mg, 10 μmol, 1.0 equiv.) and DIPEA (3.40 μL, 20 μmol, 2 equiv.) dissolved in 1 mL DMF were added and the resin was shaken at room temperature for 23 h. After filtration the resin was washed with 3×DMF and 3×DCM and cleavage cocktail DCM/TFA 1:1 (1 mL) was added and shake for 15 min. The resin was filtered off washed with 1 mL DCM and the combined filtrate was added to 10 mL of an ice-cooled mixture of MTBE and n-hexane (1:1). The resin was treated two more times with cleavage cocktail and each individual filtrate was dropped into 10 mL MTBE/hexane. The precipitates were isolated by centrifugation. The combined pellets were resuspended in 10 mL MTBE and isolated again by centrifugation and dried in vaccuo afterwards. Purification by prep. HPLC resulting in 6.78 mg (32% based on intermediate 1.22) intermediate II.43 as colorless lyophilizate. HPLC (method AF): t_{R}=15.5 min. MS (ESI): found m/z=991.4 [M+2H]²⁺, calcd. m/z=991.0 [C₈₈H₁₃₉N₂₃O₂₇S]²⁺.

### Synthesis of III.43

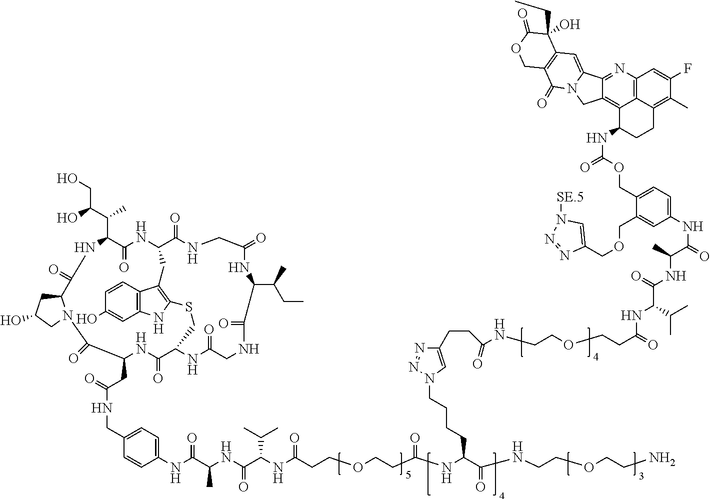

Intermediate III.43 was synthesized according to GP2 using intermediate II.43 (5.43 mg, 2.59 μmol, 1.0 equiv.) and intermediate 4.3 (17.00 mg, 6.22 μmol, 2.4 equiv.) in DMF (342 μL) with 31 μL aqueous catalyst solution containing 100 mM copper (II) sulfate pentahydrate, 120 mM tris(3-hydroxypropyltriazolylmethyl)amine and 300 mM sodium ascorbate. After 1 h the reaction was complete, filtered through a 0.2 μm membrane filter and purified by prep. HPLC to yield intermediate III.43 as a faint yellowish lyophilizate (6.23 mg, 32%). HPLC (method AG, 380 nm): t_{R}=19.4 min. MS (ESI): found m/z=1863.5 [M+4H]⁴⁺, calcd. m/z=1862.8 [C₃₃₀H₄₉₇F₂N₄₅O₁₄₃S]⁴⁺.

### Synthesis of IV.43

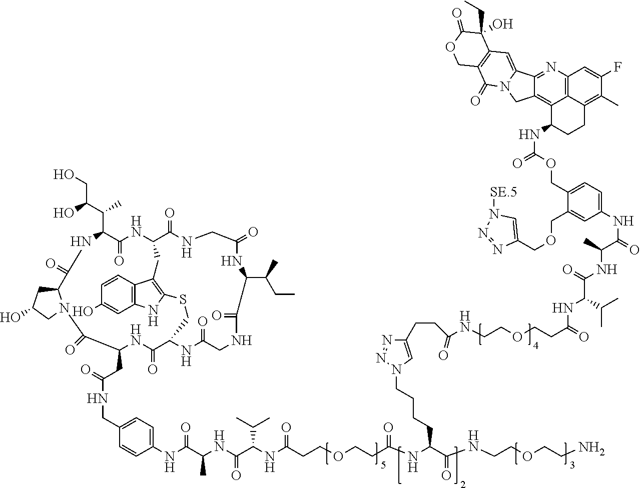

Compound IV.43 was synthesized according to GP4 using the TFA-salt of intermediate III.43 (6.23 mg, 0.82 μmol, 1 equiv.), Mal-PEG4-NHS-ester solution, 100 mM in DMF (16.5 μL, 1.65 μmol, 2 equiv.) and DIPEA solution, 100 mM in DMF (16.5 μL, 1.65 μmol, 2 equiv.) in DMF (82 μL). Upon completion after 5.5 h, the reaction mixture added dropwise to 5 mL of ice-cooled MTBE and the precipitate was collected by centrifugation (4000×g, 4 min) and washed with 5 mL fresh MTBE. The crude was dissolved in 400 μL DMF, filtered through a 0.2 μm membrane filter (Nylon) and purified by RP-HPLC to afford compound IV.43 (4.28 mg, 66%) as faint yellowish lyophilizate. HPLC (method J): t_{R}=12.96 min. MS (ESI): found m/z=1570.47581 [M+5H]⁵⁺, calcd. m/z=1570.0906 [C₃₄₈H₅₂₄F₂N₄₇O₁₅₁S]⁵⁺.

### Synthesis of II.44

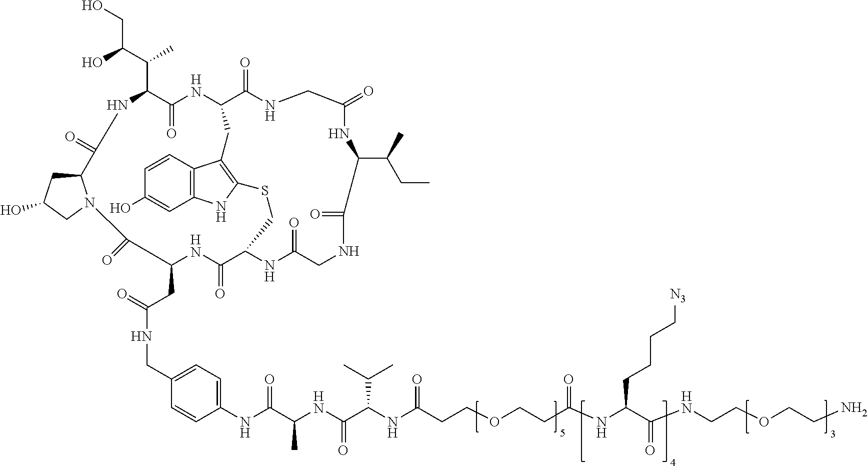

200 mg (approx. 40 μmol, 4 equiv.) resin loaded intermediate 2.6 was treaded with the same procedure as described for intermediate 4368 to obtain 11.60 mg (49% based on intermediate 1.22) intermediate II.44 as colorless lyophilizate. HPLC (method AF): t_{R}=18.5 min. MS (ESI): found m/z=1145.5 [M+2H]²⁺, calcd. m/z=1145.6 [C₁₀₀H₁₅₉N₃₁O₂₉S]²⁺.

### Synthesis of III.44

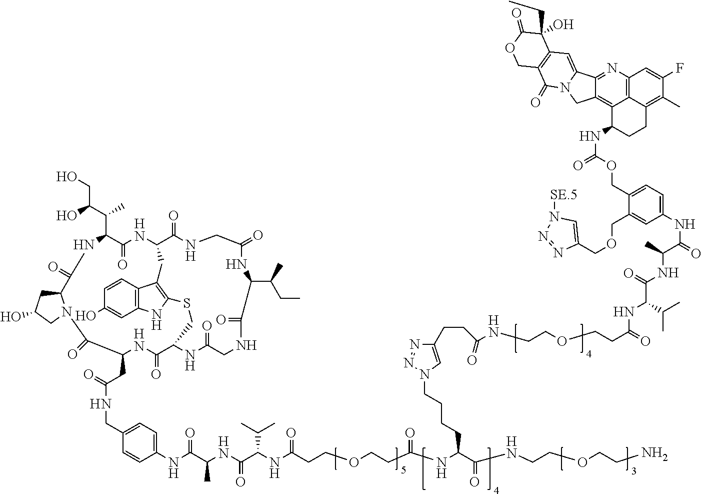

Intermediate III.44 was synthesized according to GP2 using intermediate II.44 (5.50 mg, 2.29 μmol, 1.0 equiv.) and Intermediate 4.3 (30.00 mg, 10.97 μmol, 4.8 equiv.) in DMF (548 μL) with 54.9 μL aqueous catalyst solution containing 100 mM copper (II) sulfate pentahydrate, 120 mM tris(3-hydroxypropyltriazolylmethyl)amine and 300 mM sodium ascorbate. After 1 h the reaction was complete, filtered through a 0.2 μm membrane filter and purified by prep. HPLC to yield intermediate III.44 as a faint yellowish lyophilizate (12.87 mg, 42%). HPLC (method AG, 380 nm): t_{R}=21.7 min. MS (ESI): found m/z=1890.8 [M+7H]⁷⁺, calcd. m/z=1889.8 [C₅₈₄H₈₇₆F₄N₇₅O₂₆₁S]⁷⁺.

### Synthesis of IV.44

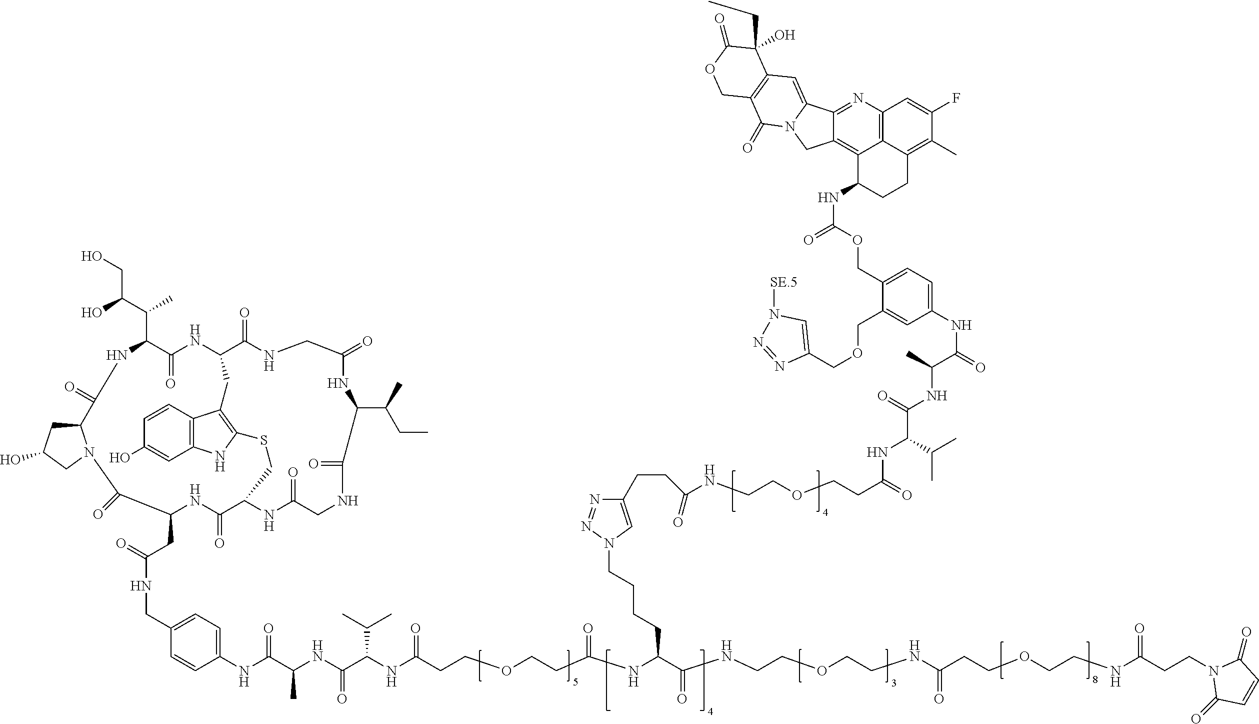

Compound IV.44 was synthesized according to GP4 using the TFA-salt of intermediate III.44 (12.87 mg, 0.96 μmol, 1 equiv.), Mal-PEG₃-NHS-ester solution, 100 mM in DMF (19.3 UL, 1.93 μmol, 2 equiv.) and DIPEA solution, 100 mM in DMF (19.3 μL, 1.93 μmol, 2 equiv.) in DMF (192 μL). Upon completion after 5.5 h, the reaction mixture added dropwise to 5 mL of ice-cooled MTBE and the precipitate was collected by centrifugation (4000×g, 4 min) and washed with 5 mL fresh MTBE. The crude was dissolved in 400 μL DMF, filtered through a 0.2 μm membrane filter (Nylon) and purified by RP-HPLC to afford compound IV.44 (10.03 mg, 75%) as faint yellowish lyophilizate. HPLC (method J): t_{R}=13.2 min. MS (ESI): found m/z=2300.99 [M+6H]⁶⁺, calcd. m/z=2300.33 [C₆₁₀H₉₁₇F₄N₇₇O₂₇₃S]⁶⁺.

### Synthesis of II.45

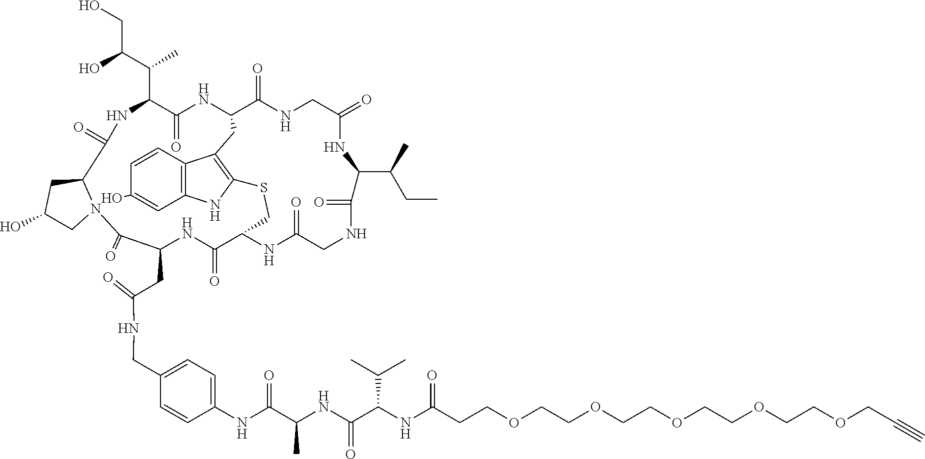 [SMILES: C#CCOCCOCCOCCOCCOCCC(=O)N[C@H](C(=O)N[C@@H](C)C(=O)Nc1ccc(CNC(=O)C[C@@H]2NC(=O)[C@@H]3CSc4[nH]c5cc(O)ccc5c4C[C@H](NC(=O)[C@H]([C@@H](C)[C@@H](O)CO)NC(=O)[C@@H]4C[C@@H](O)CN4C2=O)C(=O)NCC(=O)N[C@@H]([C@@H](C)CC)C(=O)NCC(=O)N3)cc1)C(C)C]

Intermediate II.45 was synthesized according to GP3 using crude intermediate 1.22 (48 μmol, 1 equiv.), intermediate 3.1 (43 mg, 96 μmol, 2.0 equiv.) and diisopropylethylamine (16.3 μL, 96 μmol, 2.0 equiv.) in DMF (2000 μL) for the coupling reaction. Purification by RP-HPLC afforded intermediate II.41 (52 mg, 74%) as colorless solid. HPLC (method H): t_{R}=12.4 min. MS (ESI): found m/z=1464.58 [MH]⁺, calcd. m/z=1464.67 [C₆₈H₉₈N₁₃O₂₁S]²⁺.

### Synthesis of III.45

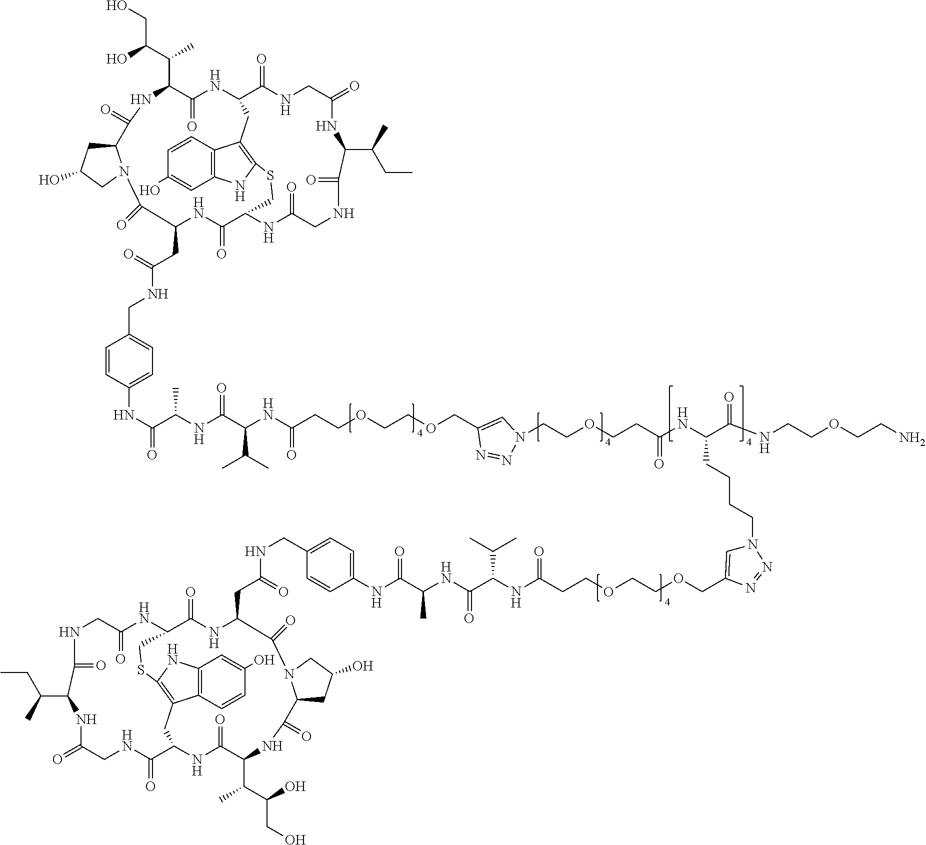

Intermediate III.45 was synthesized according to GP2 using intermediate II.45 (26 mg, 17.8 μmol, 6.0 equiv.) and intermediate 2.3 (3.2 mg, 2.96 μmol, 1.0 equiv.) in DMF (444 μL) with 88.8 μL aqueous catalyst solution containing 100 mM copper (II) sulfate pentahydrate, 120 mM tris(3-hydroxypropyltriazolylmethyl)amine and 300 mM sodium ascorbate. After 2 h the reaction was complete, filtered through a 0.2 μm membrane filter and purified by prep. HPLC to yield intermediate III.44 as a faint yellowish lyophilizate (15.9 mg, 63%). HPLC (method J, 305 nm): t_{R}=12.8 min. MS (ESI): found m/z=1682.2 [M+5H]⁵⁺, calcd. m/z=1681.8 [C₃₈₃H₅₆₉N₈₆O₁₁₇S₅]⁷⁺.

### Synthesis of IV.45

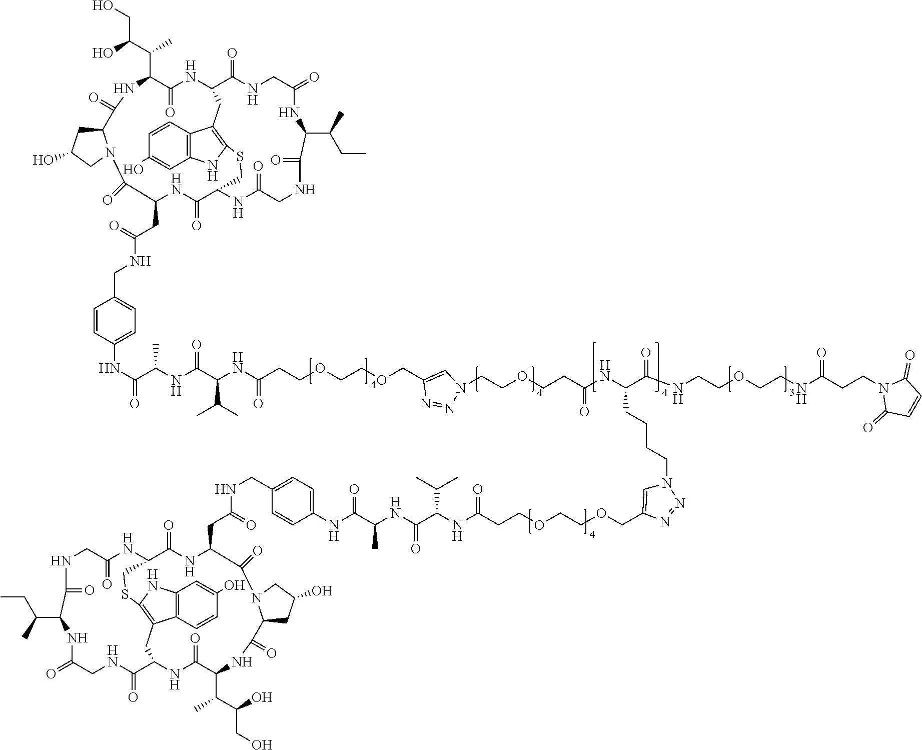

Compound IV.45 was synthesized according to GP4 using the TFA-salt of intermediate III.45 (9.03 mg, 1.06 μmol, 1 equiv.), dissolved in 500 μl DMF, BMPS (CAS: 55750-62-4, 1.13 mg, 4.24 μmol, 4 equiv.) dissolved in 100 μl and DIPEA (0.72 μL, 4.24 μmol, 4 equiv.). Upon completion after 5 h, the reaction mixture added dropwise to 5 mL of ice-cooled MTBE and the precipitate was collected by centrifugation (4000×g, 4 min) and washed with 5 mL fresh MTBE. The crude was dried in vaccuo, dissolved in 400 μL DMF/water 1:1, filtered through a 0.2 μm membrane filter (Nylon) and purified by RP-HPLC to afford compound IV.45 (4.94 mg, 54%) as colorless lyophilizate. HPLC (method J): t_{R}=13.2 min. MS (ESI): found m/z=1712.7 [M+5H]⁵⁺, calcd. m/z=1712.0 [C₃₉₀H₅₇₄N₈₇O₁₂₀S₅]⁵⁺.

### Synthesis of IV.46

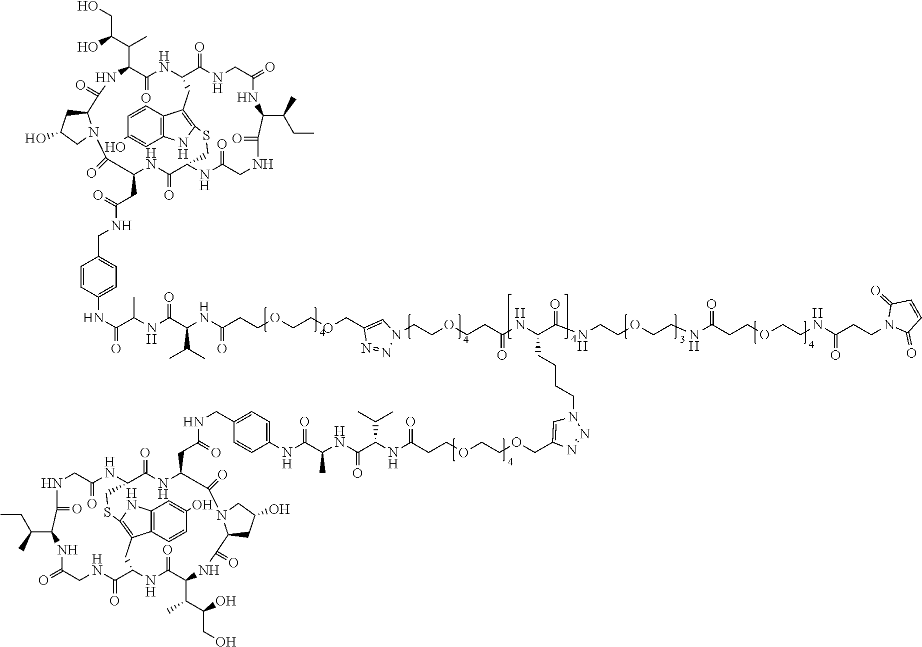

By repeating the procedure of intermediate IV.45 but with Mal-PEG4-NHS-ester (CAS: 756525-99-2) instead of BMPS the compound IV.46 was obtained in 59% yield as colorless lyophilizate. HPLC (method J): t_{R}=13.2 min. MS (ESI): found m/z=1761.8 [M+5H]⁵⁺, calcd. m/z=1761.4 [C₄₀₁H₅₉₅N₈₈O₁₂₅S₅]⁵⁺.

### Synthesis of IV.47

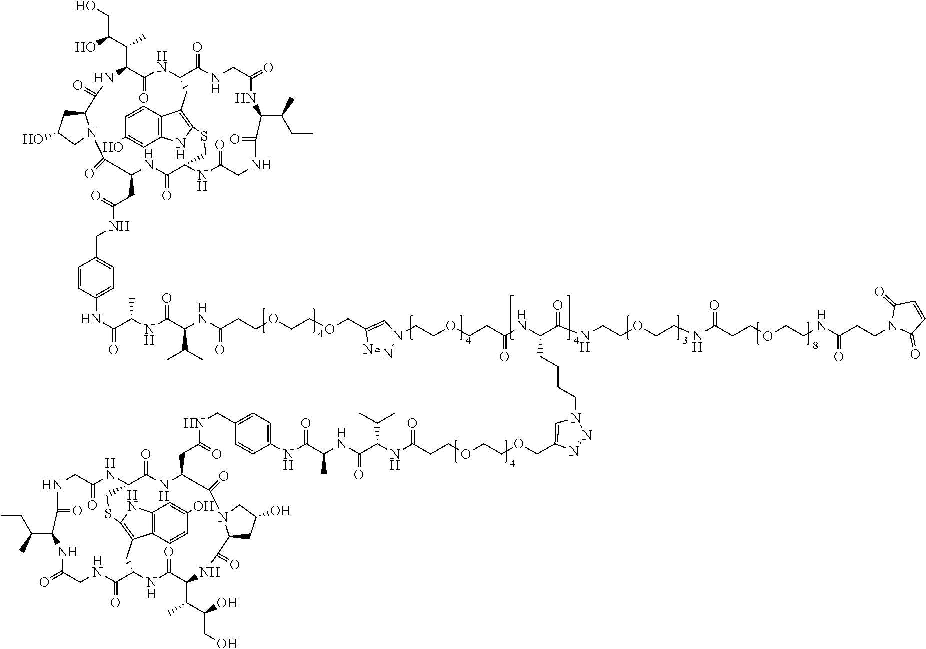

By repeating the procedure of compound IV.45 but with Mal-PEG8-NHS-ester (CAS: 756525-93-6) instead of BMPS the compound IV.47 was obtained in 44% yield as colorless lyophilizate. HPLC (method J): t_{R}=13.2 min. MS (ESI): found m/z=1797.0 [M+5H]⁵⁺, calcd. m/z=1796.7 [C₄₀₉H₆₁₁N₈₈O₁₂₉S₅]⁵⁺.

### Synthesis of IV.48

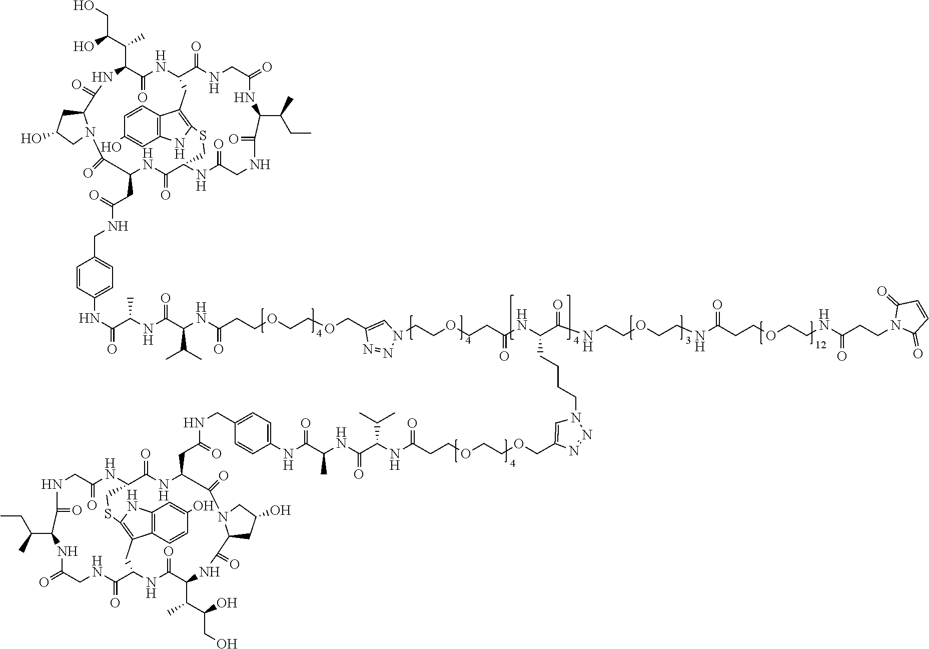

By repeating the procedure of compound IV.45 but with Mal-PEG12-NHS-ester (CAS: 2101722-60-3) instead of BMPS the compound IV.48 was obtained in 64% yield as colorless lyophilizate. HPLC (method J): t_{R}=13.3 min. MS (ESI): found m/z=1832.0 [M+5H]⁵⁺, calcd. m/z=1831.9 [C₄₁₇H₆₂₇N₈₈O₁₃₃S₅]⁵⁺.

### Synthesis of IV.49

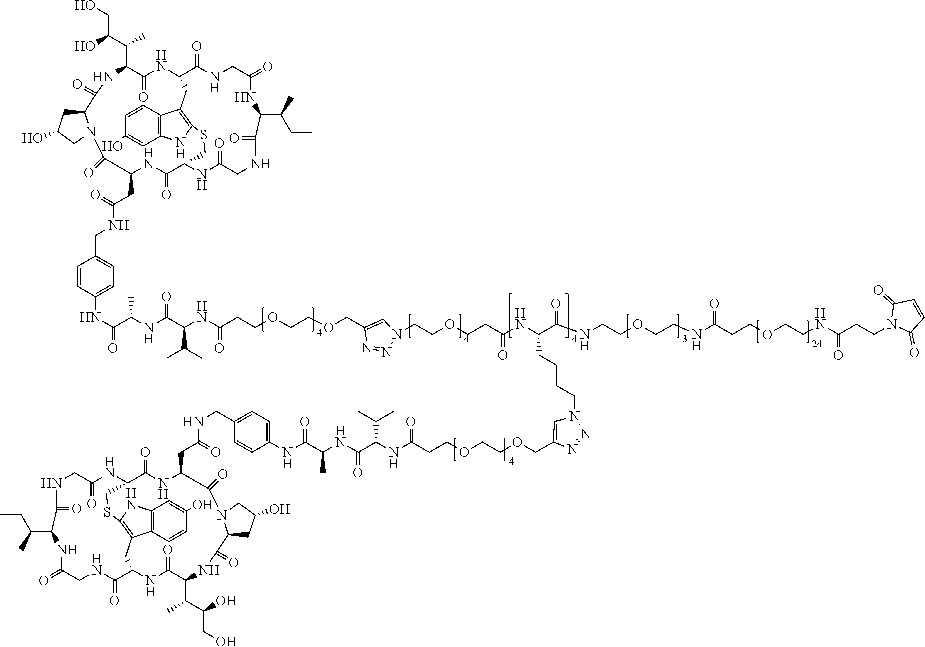

By repeating the procedure of compound IV.45 but with Mal-PEG₂₄-NHS-ester (CAS: 2226733-37-3) instead of BMPS the compound IV.49 was obtained in 64% yield as colorless lyophilizate. HPLC (method J): t_{R}=13.5 min. MS (ESI): found m/z=1937.8 [M+5H]⁵⁺, calcd. m/z=1937.7 [C₄₄₁H₆₇₅N₈₈O₁₄₅S₅]⁵⁺.

### Synthesis of III.50

Intermediate III.50 was synthesized according to GP2 using intermediate II.45 (26.0 mg, 17.8 μmol, 6.0 equiv.) and Intermediate 2.7 (4.65 mg, 2.96 μmol, 1.0 equiv.) in DMF (444 μL) with 88.8 μL aqueous catalyst solution containing 100 mM copper (II) sulfate pentahydrate, 120 mM tris(3-hydroxypropyltriazolylmethyl)amine and 300 mM sodium ascorbate. After 2 h the reaction was complete, filtered through a 0.2 μm membrane filter and purified by prep. HPLC to yield Intermediate III.50 as a colorless lyophilizate (16.9 mg, 63%). HPLC (method J, 305 nm): t_{R}=12.7 min. MS (ESI): found m/z=1780.2 [M+5H]⁵⁺, calcd. m/z=1779.7 [C₄₀₃H₆₁₂N₈₇O₁₂₉S₅]⁵⁺.

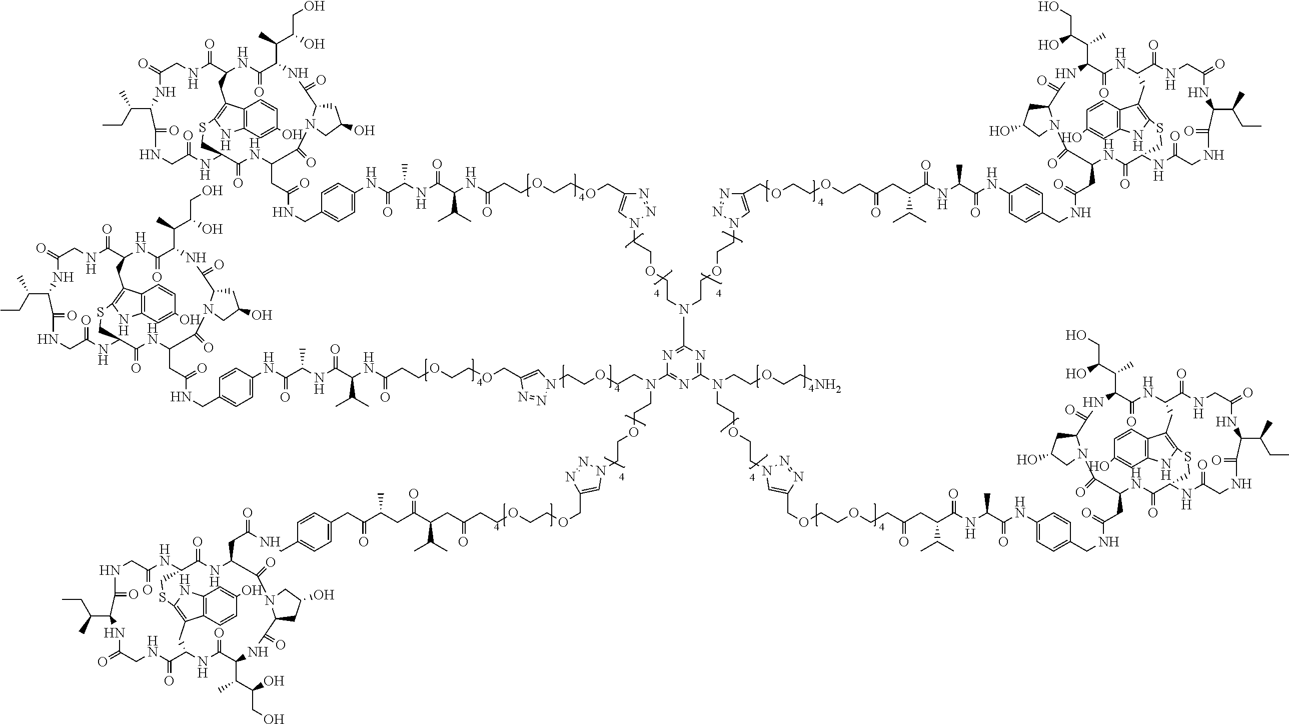

### Synthesis of IV.50

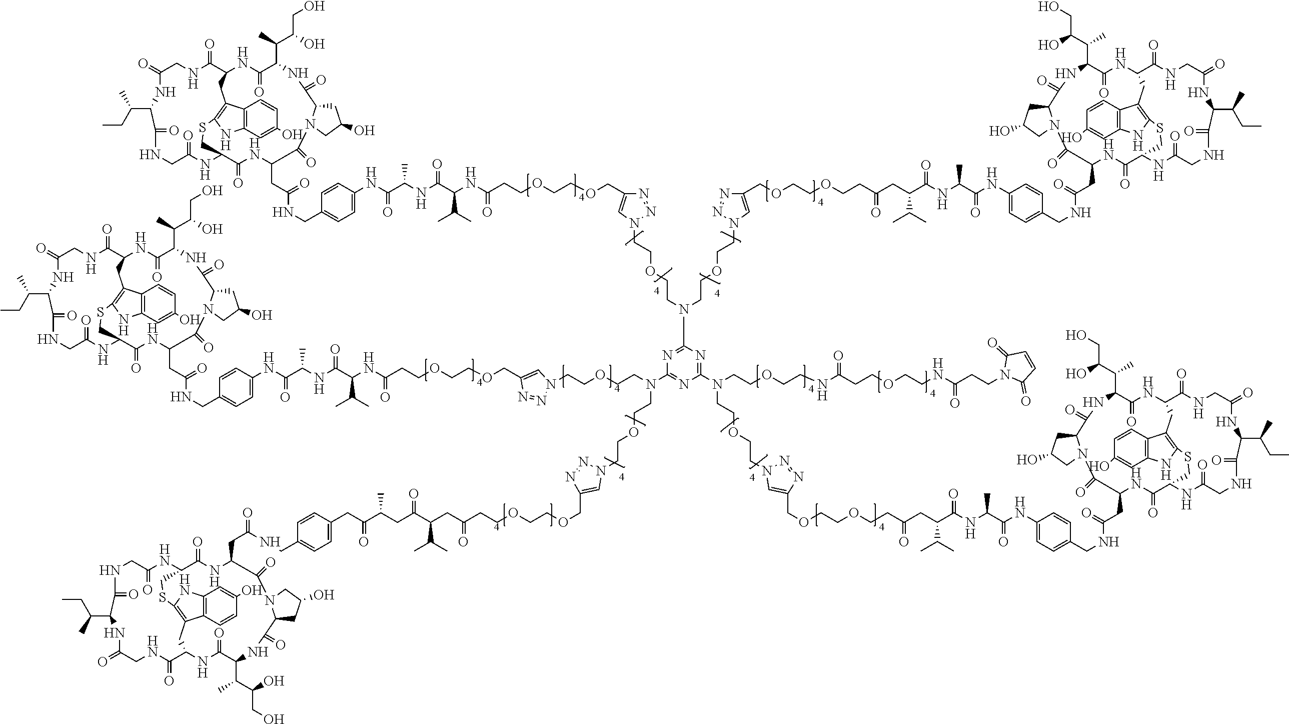

Compound IV.50 was synthesized according to GP4 using the TFA-salt of intermediate III.50 (4.22 mg, 0.47 μmol, 1 equiv.), dissolved in 500 μl DMF, Mal-PEG4-NHS-ester (CAS: 756525-99-2), 0.96 mg, 1.87 μmol, 4 equiv.) dissolved in 100 μl and DIPEA (0.32 μL, 1.87 μmol, 4 equiv.). Upon completion after 4 h, the reaction mixture was diluted with 100 μl 0.05% TFA, acidified with 5 μl 5% TFA, filtered through a 0.2 μm membrane filter (Nylon) and purified by RP-HPLC to afford compound IV.50 (3.50 mg, 80%) as colorless lyophilizate. HPLC (method J): t_{R}=13.2 min. MS (ESI): found m/z=1863.1 [M+4H+Na]⁵⁺, calcd. m/z=1863.7 [C₄₂₁H₆₃₇N₈₉O₁₃₇S₅]⁵⁺.

### Synthesis of IV.51

By repeating the procedure of intermediate IV.50 but with Mal-PEG8-NHS-ester (CAS: 756525-93-6) instead of Mal-PEG4-NHS-ester the intermediate IV51 was obtained in 72% yield as colorless lyophilizate. HPLC (method J): t_{R}=13.2 min. MS (ESI): found m/z=1894.8 [M+5H]⁵⁺, calcd. m/z=1894.7 [C₄₂₉H₆₅₄N₈₉O₁₄₁S₅]⁵⁺.

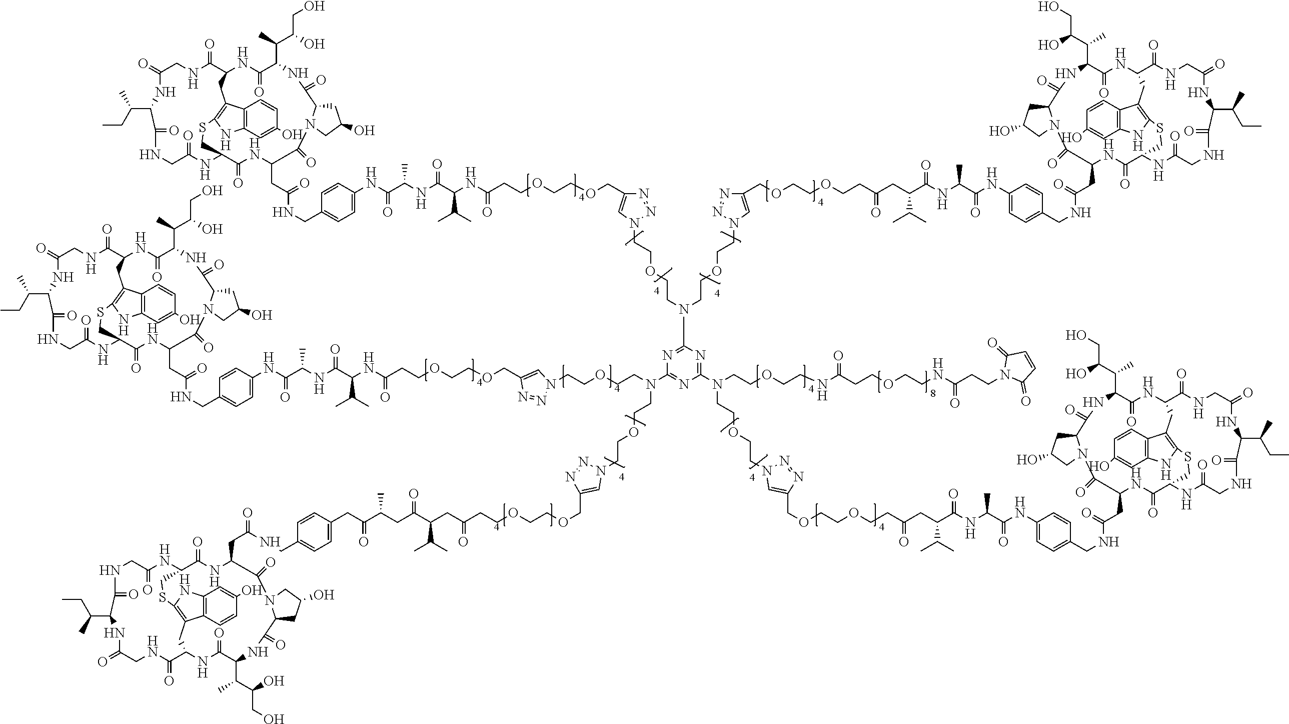

### Synthesis of IV.52

By repeating the procedure of intermediate IV.50 but with Mal-PEG12-NHS-ester (CAS: 2101722-60-3) instead of Mal-PEG4-NHS-ester the intermediate IV52 was obtained in 76% yield as colorless lyophilizate. HPLC (method J): t_{R}=13.3 min. MS (ESI): found m/z=1930.3 [M+5H]⁵⁺, calcd. m/z=1929.5 [C₄₃₇H₆₇₀N₈₉O₁₄₅S₅]⁵⁺.

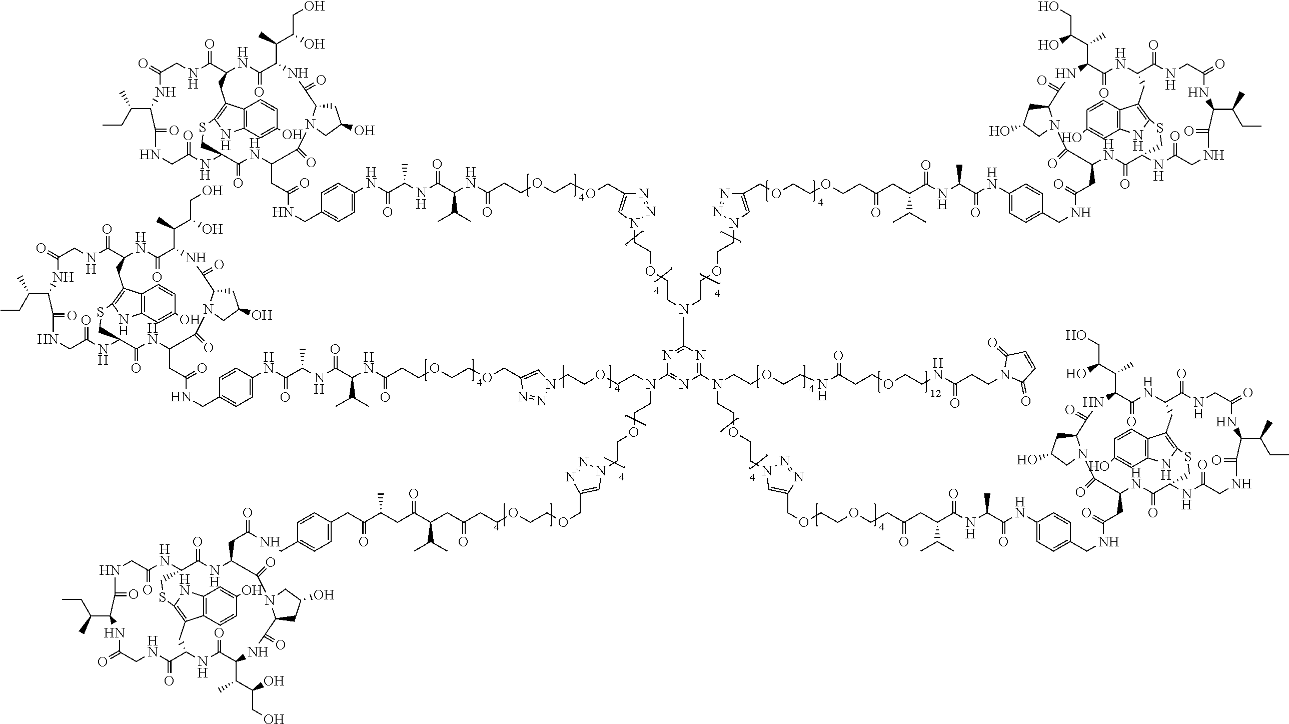

### Synthesis of IV.53

By repeating the procedure of intermediate IV.50 but with Mal-PEG₂₄-NHS-ester (CAS: 2226733-37-3) instead of Mal-PEG4-NHS-ester the intermediate IV53 was obtained in 82% yield as colorless lyophilizate. HPLC (method J): t_{R}=13.4 min. MS (ESI): found m/z=1696.7 [M+6H]⁶⁺, calcd. m/z=1696.5 [C₄₆₁H₇₁₉N₈₉O₁₅₇S₅]⁶⁺.

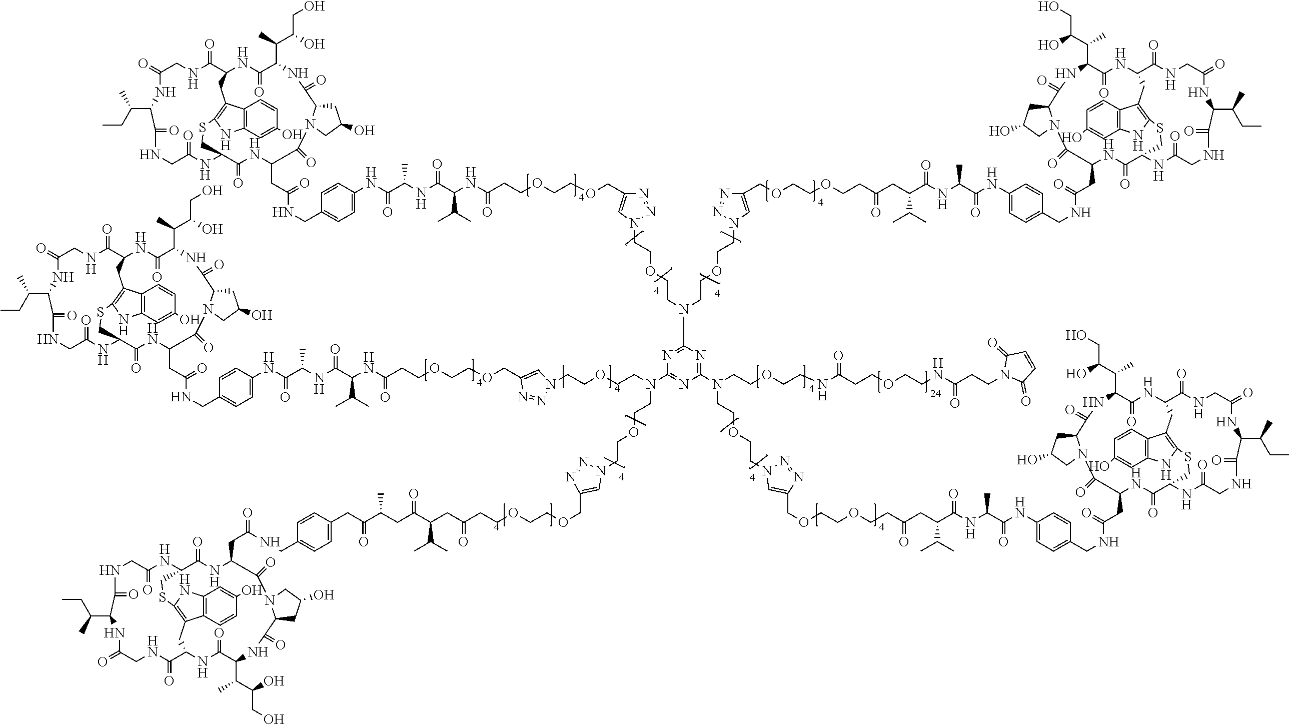

### Example 50—In Vitro Cytotoxicity Assays Using 2D and 3D Cell Cultures

Cytotoxicity of anti-GUCY2C ADCs with modular linker compounds of the present disclosure was assessed by using in vitro cytotoxicity assay with target-positive HEK293-GUCY2C and target-negative HEKwt cells based on 2D and 3D cell culture.

At the start of the 2D cytotoxicity assay, 2×10³ target-positive or target-negative cells were seeded per well in 96 Well plates and were incubated at 37° C. and 5% CO₂. Indicated ADCs were added 24 h later at various concentrations ranging from 1×10⁻⁶ M to 1.28×10⁻¹² M to the adherent cells before the plates were incubated for additional 4 days at 37° C. and 5% CO₂. Cytotoxicity was assessed by using Cell Proliferation ELISA, BrdU (colorimetric) of Roche.

At the start of the 3D cytotoxicity assay, 2×10³ target-positive or target-negative cells were seeded per well in ultra-low adhesion (ULA) 96-Well plates and were incubated at 37° C. and 5% CO₂. Spheroids were treated after 48 h with the indicated ADCs at various concentrations ranging from 1×10⁻⁶ M to 1.28×10⁻¹² M before the plates were incubated for additional 5 days at 37° C. and 5% CO₂. Cytotoxicity was assessed by using CellTiter-Glo® 2.0 Cell Viability Assay of Promega.

### Example 51—Study Design In Vivo Animal Models

Female NOD SCID mice were inoculated subcutaneously with 5×10⁶ GCC-overexpressing human embryonic kidney HEK293-GUCY2C-(HDP)-2B3 cells in 200 μL RPMI medium containing 50% Matrigel without phenol red per animal into their right flanks. Once a mean tumor volume of approximately 150 mm³ was reached, animals were allocated to control or treatment groups according to tumor size. On the same day (day 0) or the day after (day 1), the animals were treated with either a single intravenous dose of vehicle control or anti-GCC ADC conjugated to the respective modular linker compound of the present disclosure, indicated as (“IV.x”), or once a week for three weeks. The tumor volume was measured twice per week by caliper and body weights were determined in parallel. Clinical signs and survival were monitored daily. The animals were sacrificed, and necropsy was performed when one or more termination criteria arose or at study termination. The indicated dosing of the respective ADCs corresponds to the dose of total ADC in mg/kg as indicated in each case.

Many modifications and other embodiments of the present disclosure set forth herein will come to mind to the one skilled in the art to which the present disclosure pertains having the benefit of the teachings presented in the foregoing description and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### Example 52—Cytotoxicity Against Different Cell Lines

### Cytotoxicity Against NCI-N87

Table 1: In 96 h BrdU ELISA on Her2 and Trop2 expressing NCI-N87 cells, anti-Her2 ADCs and anti-Trop2 ADC showed cytotoxicity with EC50 values between 1.705×10⁻⁹ M and 3.279×10⁻¹² M.

| T-LALA-D265C | EC50 [M] |
|---|---|
| Compound IV.35 | 3.632 × 10⁻¹⁰ |
| Compound IV.25 | 3.279 × 10⁻¹² |
| Compound IV.26 | 4.626 × 10⁻¹² |
| Compound IV.27 | 1.749 × 10⁻¹¹ |
| Compound IV.30 | 5.202 × 10⁻¹⁰ |
| Compound IV.23 | 1.705 × 10⁻⁹ |
| Compound IV.43 | 4.388 × 10⁻¹¹ |
| Compound IV.44 | 4.383 × 10⁻¹¹ |
| Compound IV.39 | 4.335 × 10⁻¹⁰ |
| Compound IV.41 | 4.273 × 10⁻¹⁰ |
| Compound IV.40 | 8.659 × 10⁻¹⁰ |
| Compound IV.42 | 1.056 × 10⁻⁹ |
| Compound IV.28 | 7.702 × 10⁻¹⁰ |
| Compound IV.29 | 2.937 × 10⁻¹⁰ |

| hRS7-LALA-D265C | EC50 [M] |
|---|---|
| Compound IV.24 | 9.16 × 10⁻¹² |

### Cytotoxicity Against BxPC-3

Table 2: In 96 h BrdU ELISA on Trop2 expressing BxPC-3 cells, anti-Trop2 showed cytotoxicity with EC50 values between 3.667×10⁻⁹ M and 1.558×10⁻¹² M.

| hRS7-LALA-D265C | EC50 [M] |
|---|---|
| Compound IV.25 | 1.558 × 10⁻¹² |
| Compound IV.26 | 2.351 × 10⁻¹⁷ |
| Compound IV.27 | 5.418 × 10⁻¹² |
| Compound IV.30 | 1.749 × 10⁻⁹ |
| Compound IV.31 | 3.243 × 10⁻⁹ |
| Compound IV.23 | 3.667 × 10⁻⁹ |
| | (34% rest viability) |
| Compound IV.24 | 2.233 × 10⁻¹¹ |

### Cytotoxicity Against SKBR-3

Table 3: In 96 h BrdU ELISA on Her2 and Trop2 expressing SKBR-3 cells, anti-Her2 ADCs and anti-Trop2 ADC showed cytotoxicity with EC50 values between 1.178×10⁻¹⁰ M and 8.363×10⁻¹² M.

| | EC50 [M] |
|---|---|
| T-LALA-D265C | |
| Compound IV.25 | 8.363 × 10⁻¹² |
| Compound IV.26 | 1.388 × 10⁻¹¹ |
| Compound IV.27 | 1.018 × 10⁻¹¹ |
| Compound IV.43 | 1.178 × 10⁻¹⁰ |
| Compound IV.44 | 4.154 × 10⁻¹¹ |
| hRS7-LALA-D265C | |
| Compound IV.25 | 1.148 × 10⁻¹¹ |
| Compound IV.26 | 3.687 × 10⁻¹¹ |
| Compound IV.27 | 9.177 × 10⁻¹¹ |
| Compound IV.24 | 4.662 × 10⁻¹¹ |

Table 4: In 96 h BrdU ELISA on Her2 and Trop2 expressing JIMT-1 cells, anti-Her2 ADCs and anti-Trop2 ADC showed cytotoxicity with EC50 values between 1.205×10⁻⁹ M and 1.276×10⁻¹² M.

### Cytotoxicity Against JIMT-1

| | EC50 [M] |
|---|---|
| T-LALA-D265C | |
| Compound IV.25 | 5.534 × 10⁻¹⁰ |
| Compound IV.26 | 8.002 × 10⁻¹⁰ |
| Compound IV.27 | 8.175 × 10⁻¹⁰ |
| Compound IV.43 | 5.944 × 10⁻¹⁰ |
| Compound IV.44 | 1.205 × 10⁻⁹ |
| hRS7-LALA-D265C | |
| Compound IV.25 | 1.498 × 10⁻¹⁰ |
| Compound IV.26 | 1.276 × 10⁻¹⁰ |
| Compound IV.27 | 1.366 × 10⁻¹⁰ |

### Cytotoxicity Against MDA-MB-231 (Target Negative)

Table 5: In 96 h BrdU ELISA on target negative MDA-MB-231 cells, anti-Her2 ADCs and anti-Trop2 ADC showed no cytotoxicity.

| | EC50 [M] |
|---|---|
| T-LALA-D265C | |
| Compound IV.35 | — |
| Compound IV.25 | — |
| Compound IV.26 | — |
| Compound IV.27 | — |
| Compound IV.30 | — |
| Compound IV.39 | — |
| Compound IV.41 | — |
| Compound IV.40 | — |
| Compound IV.42 | — |
| Compound IV.28 | — |
| Compound IV.29 | — |
| hRS7-LALA-D265C | |
| Compound IV.25 | — |
| Compound IV.26 | — |
| Compound IV.27 | — |

### Cytotoxicity Against HEKwt (Target Negative)

Table 6: In 96 h BrdU ELISA on target negative HEKwt cells, anti-Her2 ADCs and anti-Trop2 ADC showed no cytotoxicity.

| hRS7-LALA-D265C | EC50 [M] |
|---|---|
| Compound IV.24 | 8.665 × 10⁻⁹ |

### Cytotoxicity Against L540

Table 7: In 96 h CTG on CD30 expressing L540 cells, anti-CD30 ADC showed cytotoxicity with EC50 value 3.421×10⁻¹⁰ M.

| Brentuximab-LALA-D265C | EC50 [M] |
|---|---|
| Compound IV.32 | 3.421 × 10⁻¹⁰ |
| | (rest viability: 29%) |

### ClogP Values

Table 8 depicts the ClogP values of the payloads (D) as well as the ClogP values for compounds according to the present disclosure with Solubility Enhancers (SE) or without SEs:

| Solubility Enhancer | Payload | IV Number | ClogP |
|---|---|---|---|
| | Exatecan | | 1.54 |
| | SN-38 | | 1.87 |
| | E104 (TLR7 agonist 1) | | 4.01 |
| | MMAE | | 3.51 |
| | Triptolide | | 1.32 |
| | Doxorubicine | | 0.53 |
| | PNU-159682 | | 3.17 |
| | NAMPTi | | 2.21 |
| | Deacetylcolchicine | | 1.64 |
| | alpha-Amantin | | −7.66 |
| none | Exatecan | | 0.28 |
| PEG4 | Exatecan | IV.1 | 0.51 |
| PEG4 | Exatecan | IV.2 | −0.65 |
| PEG12 | Exatecan | IV.3 | −1.4 |
| PEG2 - β-cyclodextrin | Exatecan | IV.4 | −24.79 |
| PEG4 - β-cyclodextrin | Exatecan | IV.5 | −24.97 |
| PEG8 - β-cyclodextrin | Exatecan | IV.6 | −25.35 |
| PEG24 - β-cyclodextrin | Exatecan | IV.7 | −26.85 |
| PEG4 - β-CD | Exatecan | IV.8 | −26.14 |
| PEG8 - β-CD | Exatecan | IV.9 | −26.52 |
| PEG24 - β-cyclodextrin | Exatecan | IV.10 | −28.02 |
| PEG2 - γ-cyclodextrin | Exatecan | IV.11 | −29.5 |
| PEG4 - γ-cyclodextrin | Exatecan | IV.12 | −29.68 |
| PEG8 - γ-cyclodextrin | Exatecan | IV.13 | −30.06 |
| PEG3 - beta-CD-2-O- | Exatecan | IV.14 | −24.41 |
| verknüpft | | | |
| PEG3 - gamma-CD-2- | Exatecan | IV.15 | −27.95 |
| O-verknüpft | | | |
| Polysarcosine -acid | Exatecan | IV.16 | −18.1 |
| Polysarcosine -amid | Exatecan | IV.17 | −19.4 |
| Polysarcosine -amid | Exatecan | IV.18 | −20.56 |
| PEG4-Diglucamide | Exatecan | IV.19 | −8.98 |
| (Gluconolacton) | | | |
| PEG4-Monoglucamide | Exatecan | IV.20 | −17.05 |
| (Gluconolacton) | | | |
| PEG4-Diglucamide | Exatecan | IV.21 | −18.18 |
| (Glucamin) | | | |
| PEG8-Diglucamide | Exatecan | IV.22 | −18.55 |
| (Glucamin) | | | |
| PEG4 - γ-cyclodextrin | SN-38 | IV.23 | −29.61 |
| PEG4 - γ-cyclodextrin | PNU-159682 | IV.24 | −26.46 |
| PEG8 - β-cyclodextrin | MMAE | IV.25 | −23.00 |
| Polysarcosine -amid | MMAE | IV.26 | −17.05 |
| PEG8-Diglucamide | MMAE | IV.27 | −15.04 |
| (Glucamin) | | | |
| PEG4 - γ-cyclodextrin | Deacetylcholchicin | IV.28 | −29.49 |
| PEG4 - γ-cyclodextrin | Deacetylcholchicin | IV.29 | −55.52 |
| PEG8 - β-cyclodextrin | SN-38 | IV.30 | −26.96 |
| PEG8-Diglucamide | SN-38 | IV.31 | −18.99 |
| (Glucamin) | | | |
| PEG8 - β-cyclodextrin | NAMPTi 1 | IV.32 | −24.70 |
| PEG8 - β-cyclodextrin | E104 (TLR7 agonist 1) | IV.33 | −21.08 |
| Polysarcosine -amid | E104 (TLR7 agonist 1) | IV.34 | −15.13 |
| PEG8 - β-cyclodextrin | Doxorubicin | IV.35 | −26.60 |
| 2 Toxine | | | |
| PEG8 - B-cyclodextrin | Doxorubicin | IV.36 | −49.75 |
| 4Toxine_Mal-PEG4 | | | |
| PEG8 - β-cyclodextrin | Doxorubicin | IV.37 | −49.94 |
| 4Toxine_Mal-PEG8 | | | |
| 2-Alkyl_PEG4 - γ- | Doxorubicin | IV.38 | −28.03 |
| cyclodextrin | | | |
| PEG8 - γ- | Triptolide | IV.39 | −28.42 |
| cyclodextrin_2_Alkyl- | | | |
| PEG4_2 Toxine | | | |
| PEG8 - PEG8- | Triptolide | IV.41 | −19.03 |
| Diglucamide_2 Toxine | | | |
| PEG8 - PEG8- | Triptolide | IV.42 | −42.97 |
| Diglucamide_5 Toxine | | | |
| PEG8 - β-cyclodextrin | 1 × Amanitin, 2 × Exatecan | IV.43 | −31.88 |
| PEG8 - β-cyclodextrin | 1 × Amanitin, 4 × Exatecan | IV.44 | −55.13 |
| Ohne | 5 × Amanitin, Azidolysine core; BMPS | IV.45 | −30.23 |
| Ohne | 5 × Amanitin, Azidolysine core; Mal-dPEG4 | IV.46 | −31.29 |
| Ohne | 5 × Amanitin, Azidolysine core; Mal-dPEG8 | IV.47 | −31.48 |
| Ohne | 5 × Amanitin, Azidolysine core; Mal-dPEG12 | IV.48 | −31.66 |
| Ohne | 5 × Amanitin, Azidolysine core; Mal-dPEG24 | IV.49 | −32.23 |
| Ohne | 5 × Amanitin, Triazol core; Mal-dPEG4 | IV.50 | −30.14 |
| Ohne | 5 × Amanitin, Triazol core Mal-dPEG8 | IV.51 | −30.33 |
| Ohne | 5 × Amanitin, Triazol core; Mal-dPEG12 | IV.52 | −30.51 |
| Ohne | 5 × Amanitin, Triazol core; Mal-dPEG24 | IV.53 | −31.08 |
| Glucuronid | Exatecan | IV.54 | −2.92 |

1. A modular linker compound of Formula (I) 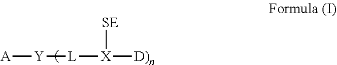
or a pharmaceutically acceptable salt or solvate thereof;
wherein:
X is self-immolative spacer 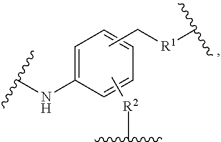
wherein:
R¹ covalently links X to D in para, meta or ortho to the amino group of X and is a bond, 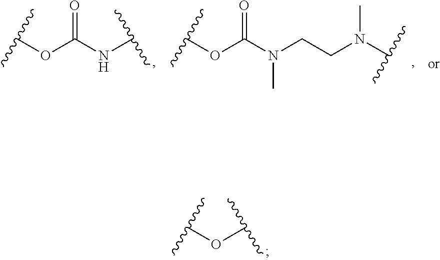
and
R² connects X to SE and is 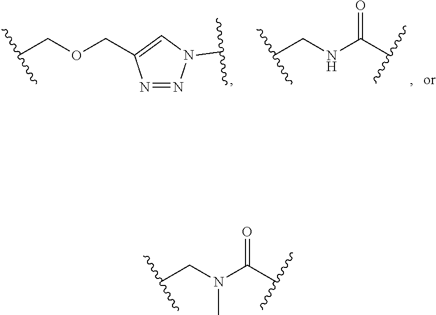
or —O—;
D is a pharmaceutically active substance;
SE is a solubility enhancing group;
L is a spacer comprising a peptide covalently linked to X;
n is an integer of at least 2, optionally n is an integer from 2 to 5;
Y is a branching unit, and
A is a reactive moiety for conjugation to a target-binding moiety.

2. The modular linker compound of claim 1, wherein
L is 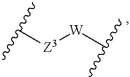
wherein:
W is a cleavable or non-cleavable peptide sequence connecting Z³ and X and is selected from the group consisting of:
Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gln, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gln, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and
wherein Z³ is a bond or a spacer comprising a C₁₋₂₀ alkyl chain or a 1 to 10 polyethylene glycol (PEG)₁₋₁₀ or a C₂-amide-PEG₁₋₁₀;
optionally L is

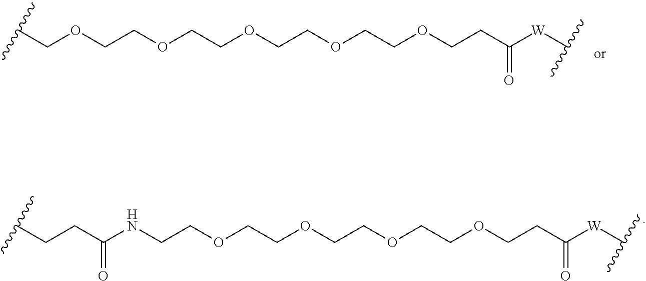

3. The modular linker compound of claim 1, wherein
Y is: 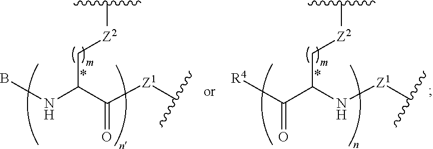
m is an integer from 1 to 6;
Z¹ connects Y to A and is 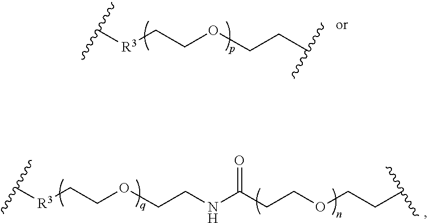
wherein
R³ is —O—, —NH—, —C(═O)—, —C(═O)N(CH₃)—, —C(═O) NH—, —NHC(═O)—, or —N(CH₃)C(═O)—, and
p is an integer from 1 to 24, optionally from 4 to 24, optionally from 8 to 24,
q is an integer from 1 to 8,
r is an integer from 1 to 24
Z² connects Y to Land is selected from the group consisting of: 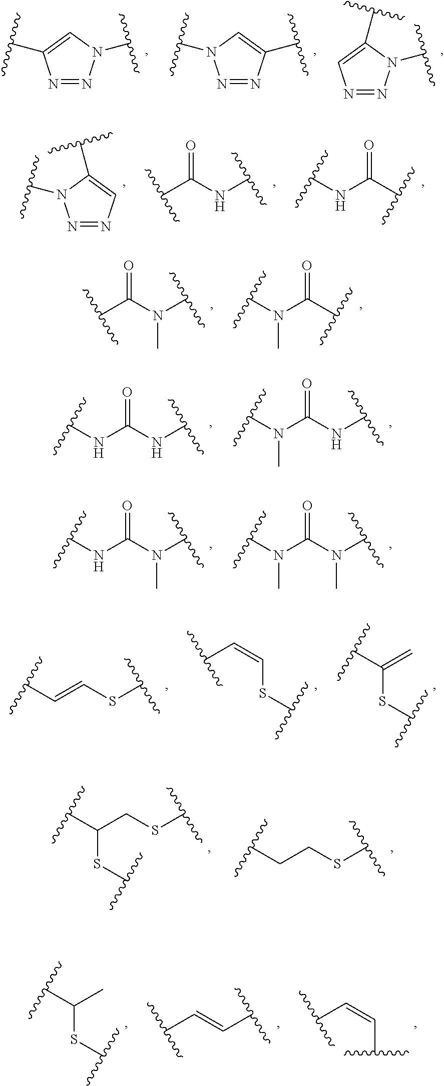 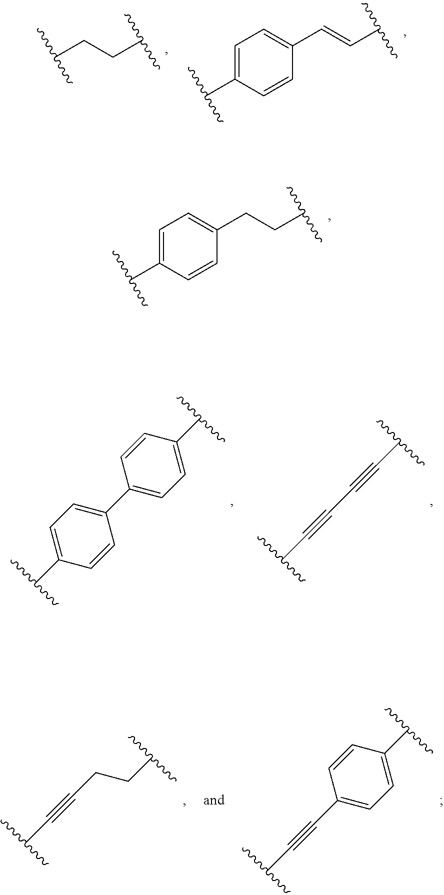
B is:
a cap, optionally an acyl group, and n′ is n; or 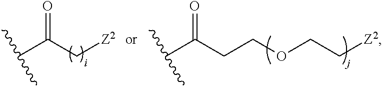
wherein
i is an integer from 1 to 20,
j is an integer from 1 to 10, and
n′ is n minus 1; or 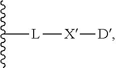
wherein
D′ is a pharmaceutically active substance different from D;
X′ is self-immolative spacer 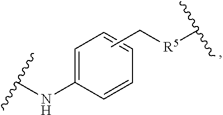
wherein:
R⁵ covalently links X′ to D′ in para or ortho to the amino group of X′ and is a bond, 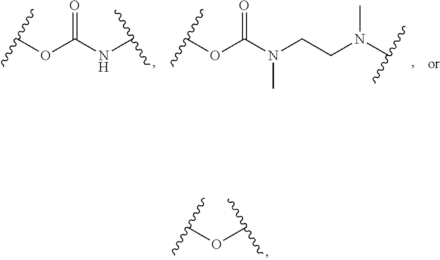
and n′ is n; and
R⁴ is OH or NH₂.

4. The modular linker compound of claim 1, wherein
Y is: 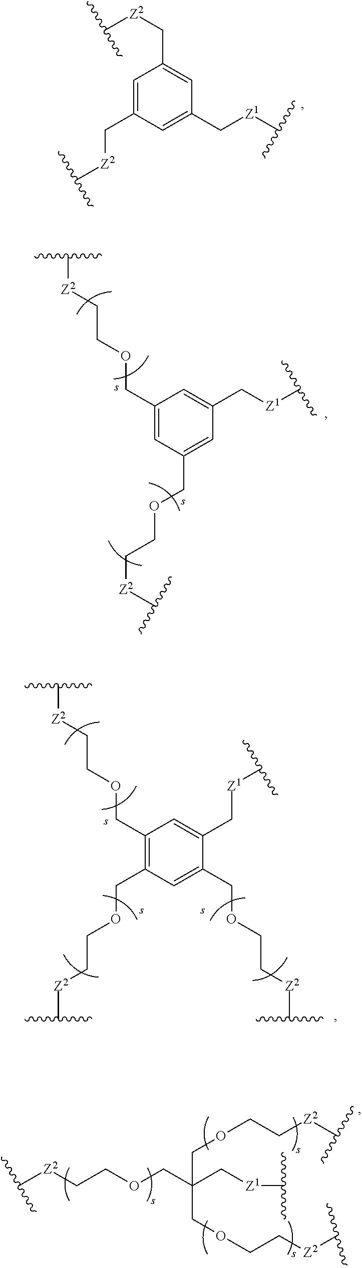 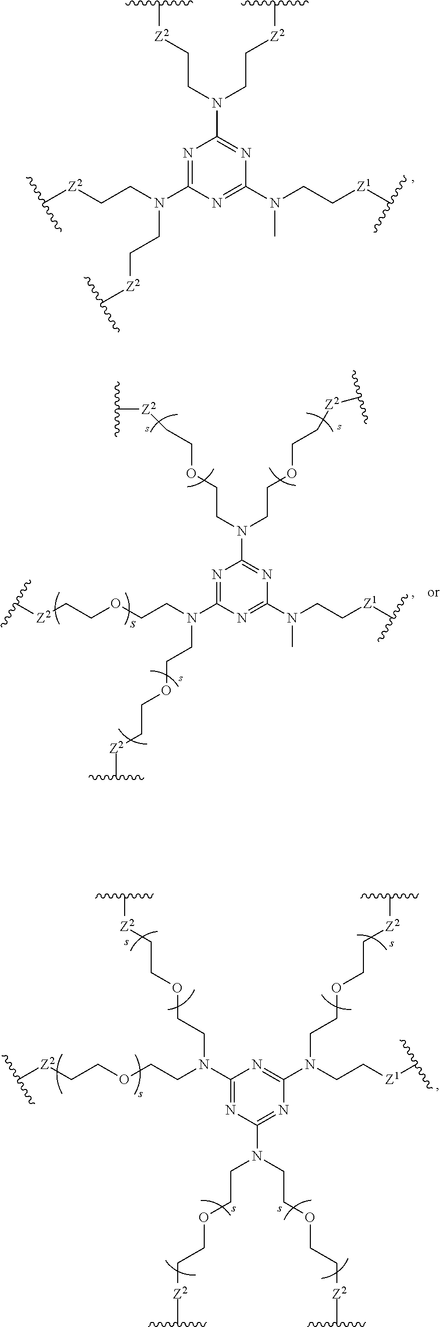 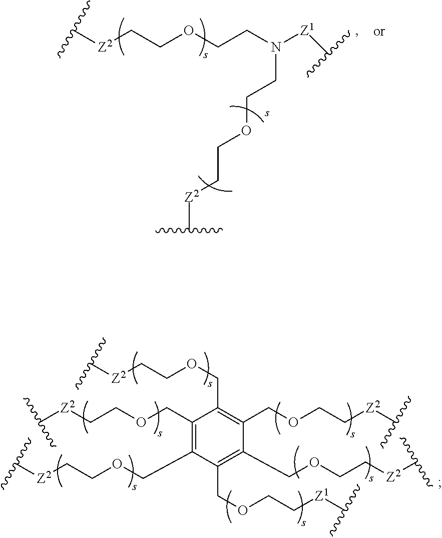
Z¹ connects Y to A and is 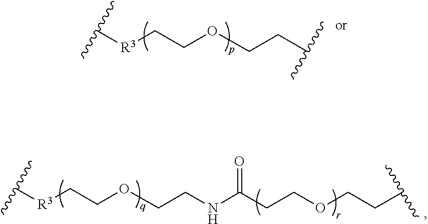
wherein
R³ is —O—, —NH—, —C(═O)—, —C(═O)N(CH₃)—, —C(═O) NH—, —NHC(═O)—, or —N(CH₃) C(═O)—,
p is an integer from 1 to 24,
q is an integer from 1 to 8,
r is an integer from 1 to 24,
s is an integer from 0 to 4,
Z² connects Y to L and is selected from the group consisting of:

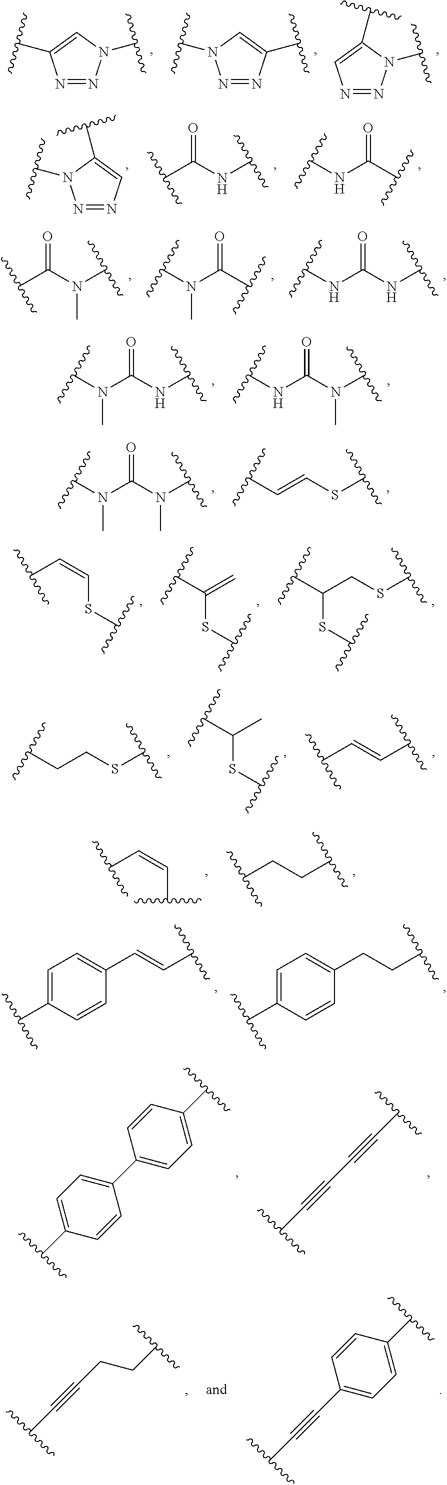

5. The modular linker compound of claim 1, wherein D and D′ are independently selected from the group consisting of: camptothecins, optionally exatecan, SN38 and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; receptor 7 toll-like (TLR7) agonists; nicotinamide phosphoribosyltransferase (NAMPT) inhibitor (NAMPTᵢ): pyrrolobenzodiazepines (PBDs); duocarmycins; anthracyclines, optionally doxorubicin; amatoxins, optionally alpha-amanitin, beta amanitin, amanin and amaninamide; cryptophycins; phalloidins; seco-cyclopropylpyrroloindoline (seco-CPI); seco-cyclopropylbenzoindoline (seco-CBI); taxols; vinblastines; colchicines; eribulins; methotrexate (MTX); triptolide; and derivatives thereof.

6. The modular linker compound of claim 1, wherein SE comprises an alpha-cyclodextrin, a beta-cyclodextrin, a gamma-cyclodextrin, a polysarcosine, a PEG, a galactoside, a glucuronide, or a polyalcohol made from carbohydrates, optionally SE comprises: 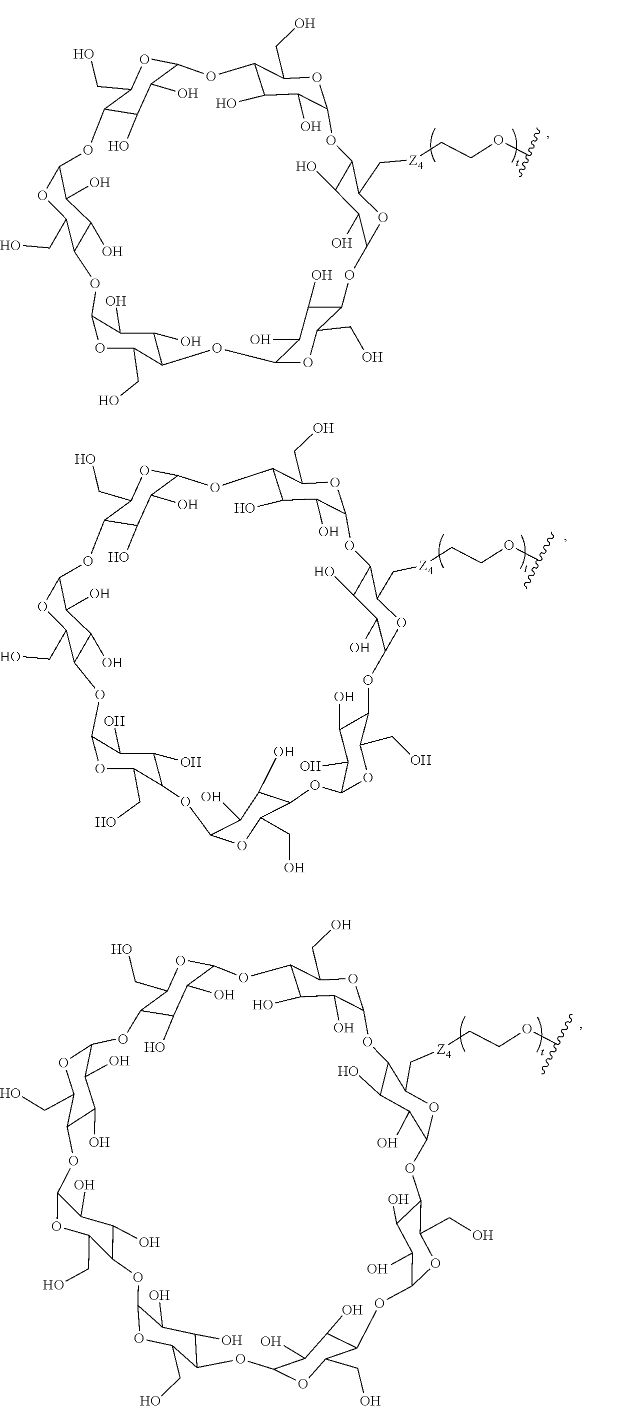 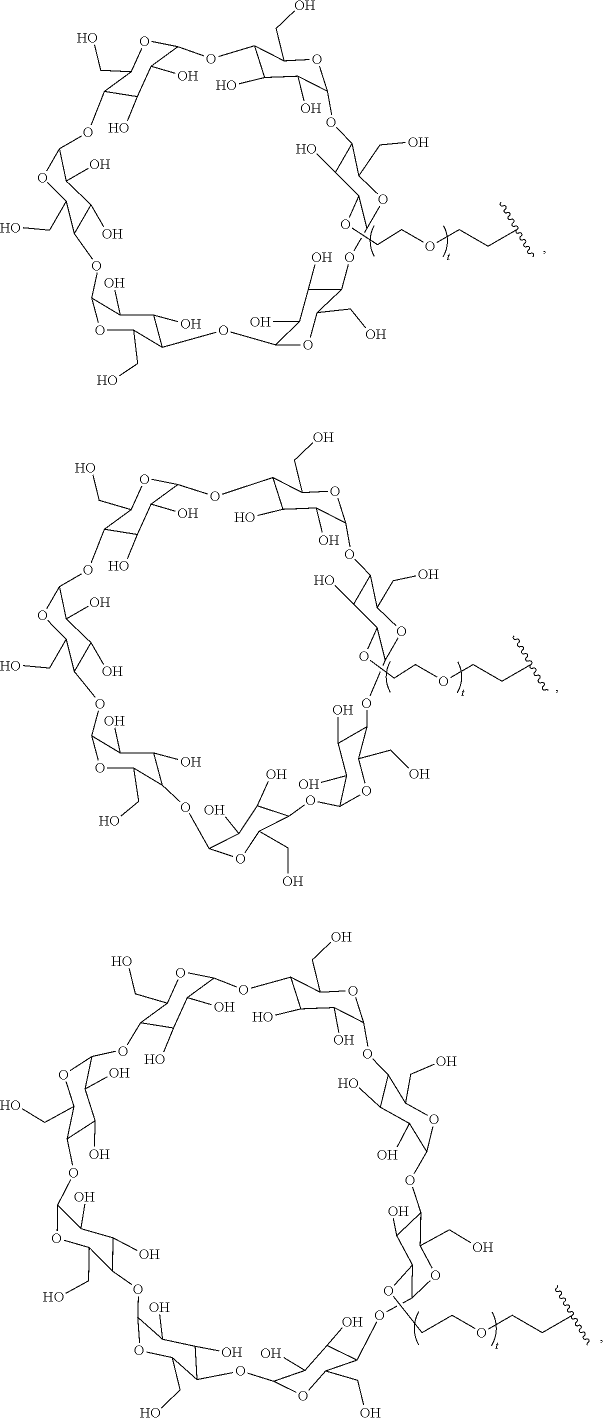 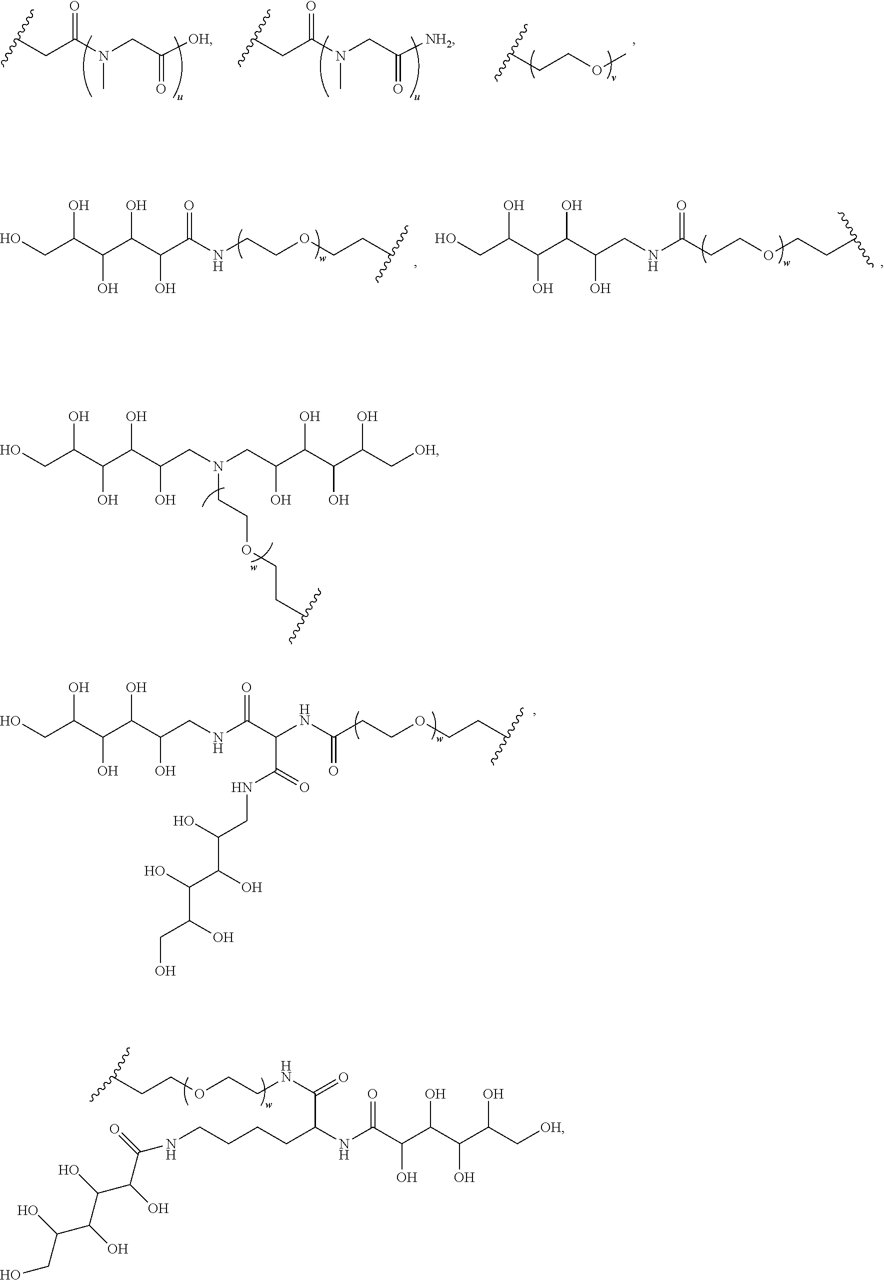 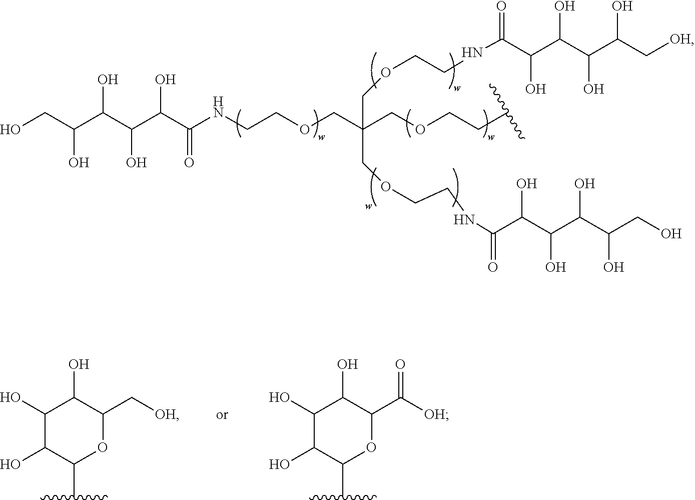
wherein Z⁴ is selected from the group consisting of: 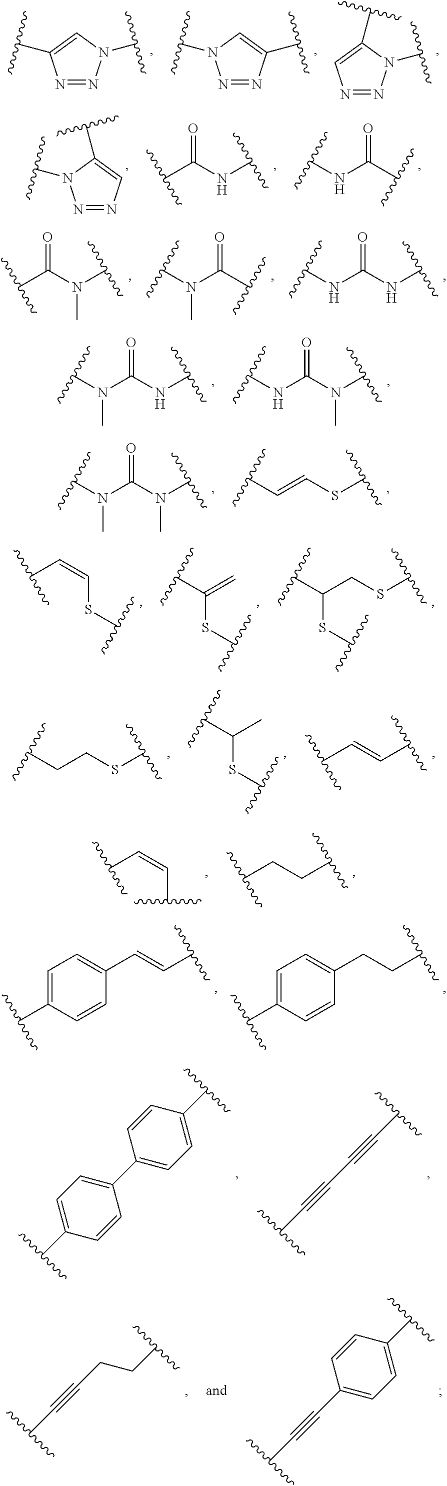
t is 2 to 18, optionally 8 to 12,
u is at least 1, optionally 10;
vis 4 to 24;
w is 2 to 24.

7. The modular linker compound of claim 1, wherein A is

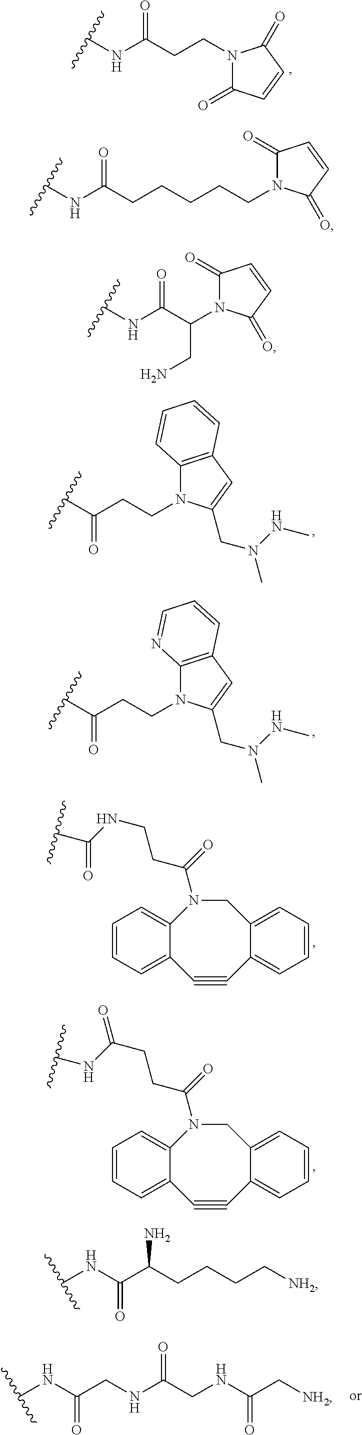

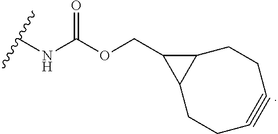 [SMILES: *NC(=O)OCC1C2CCC#CCCC21]

8. The modular linker compound of claim 1, wherein the modular linker compound is:

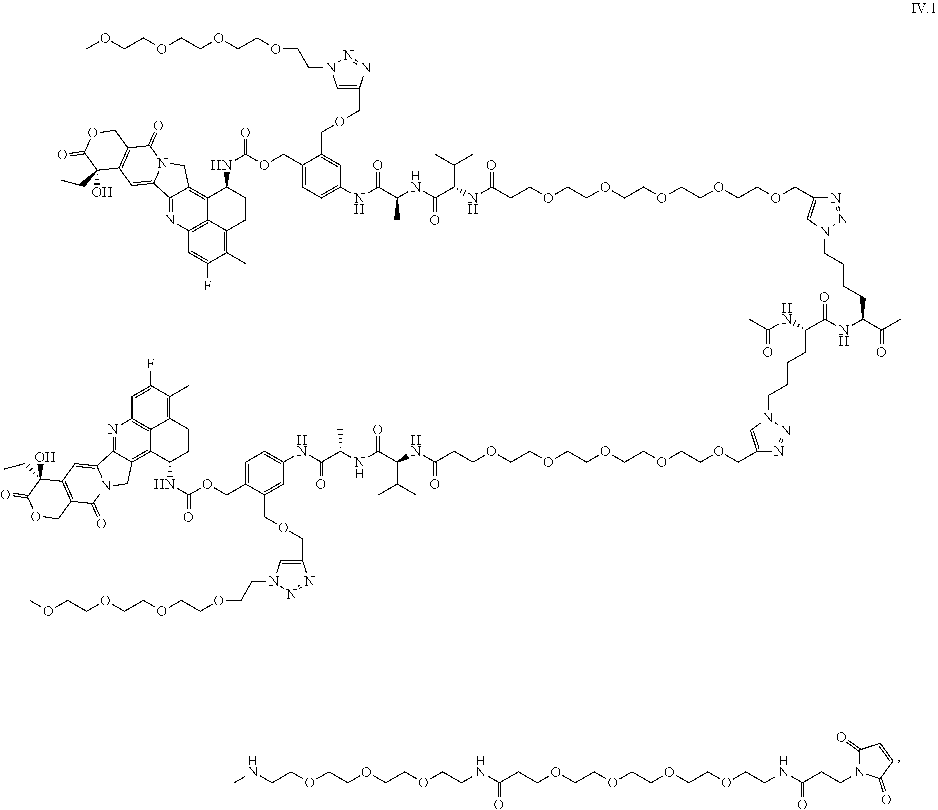

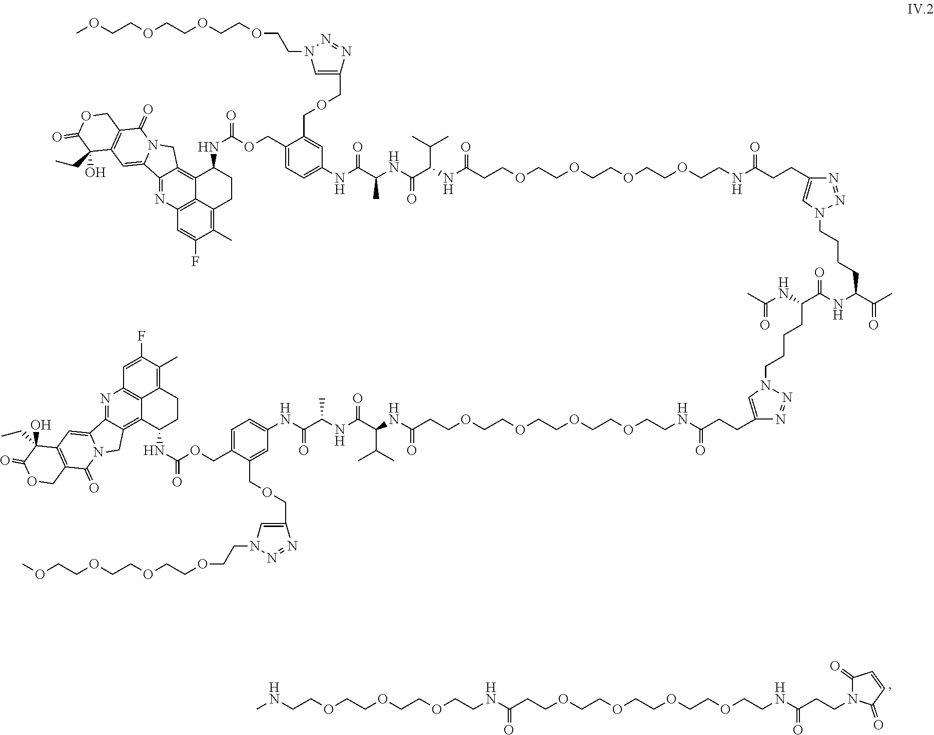

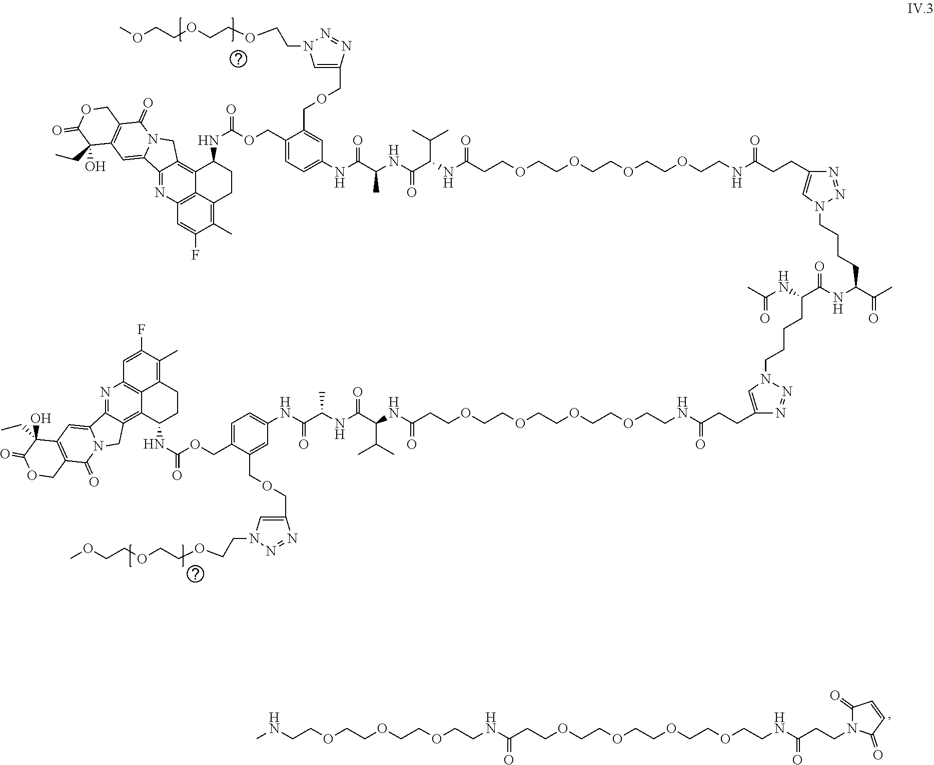

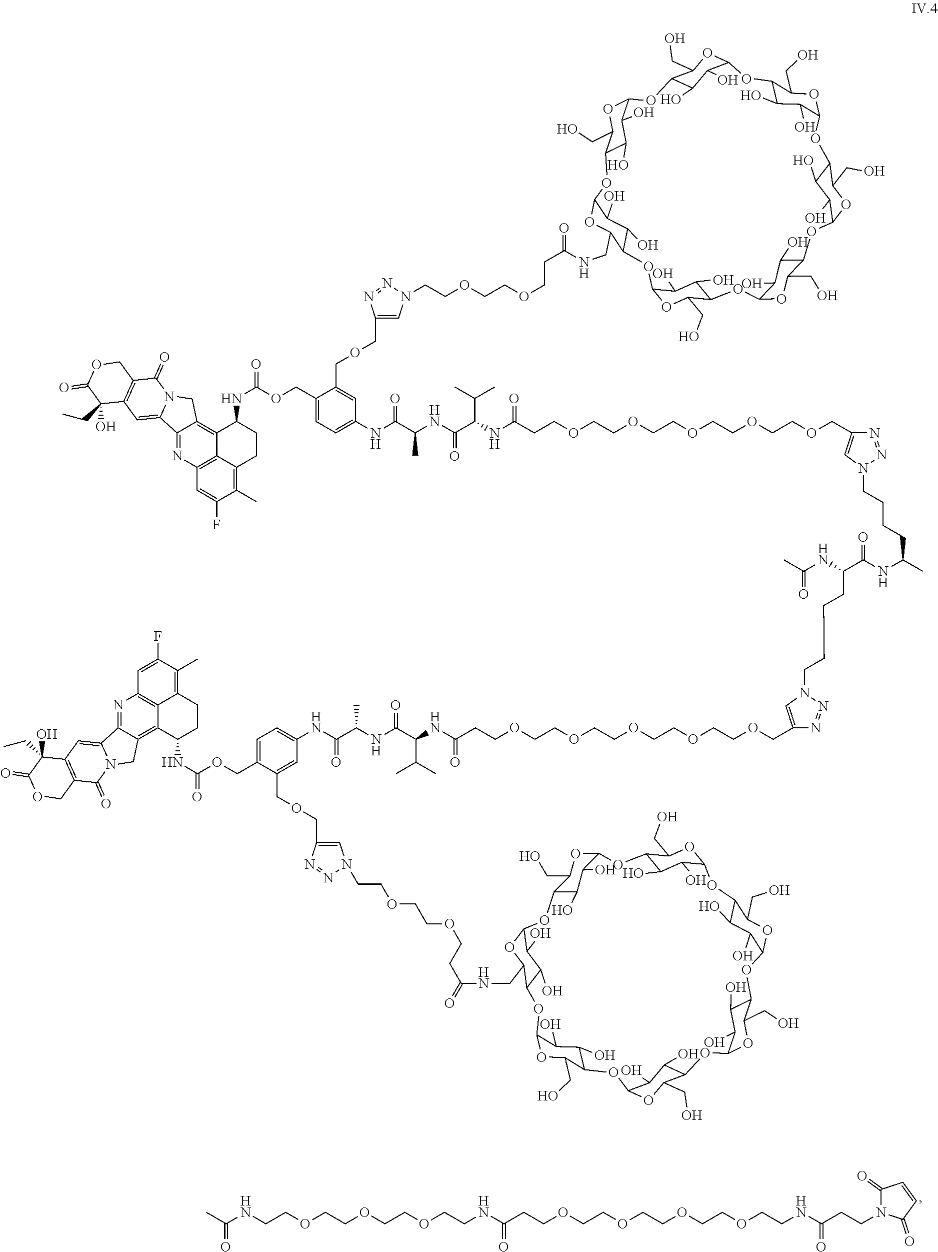

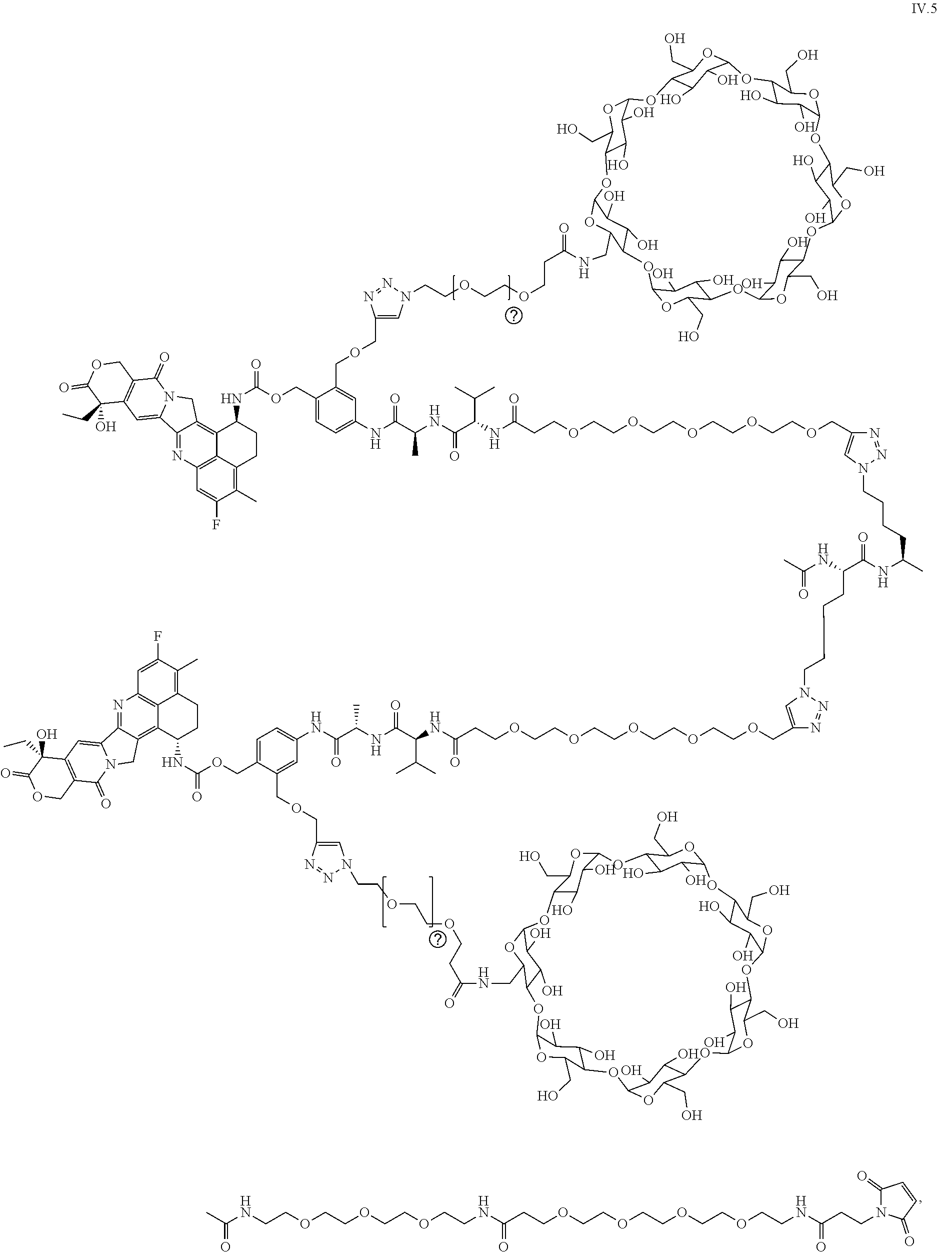

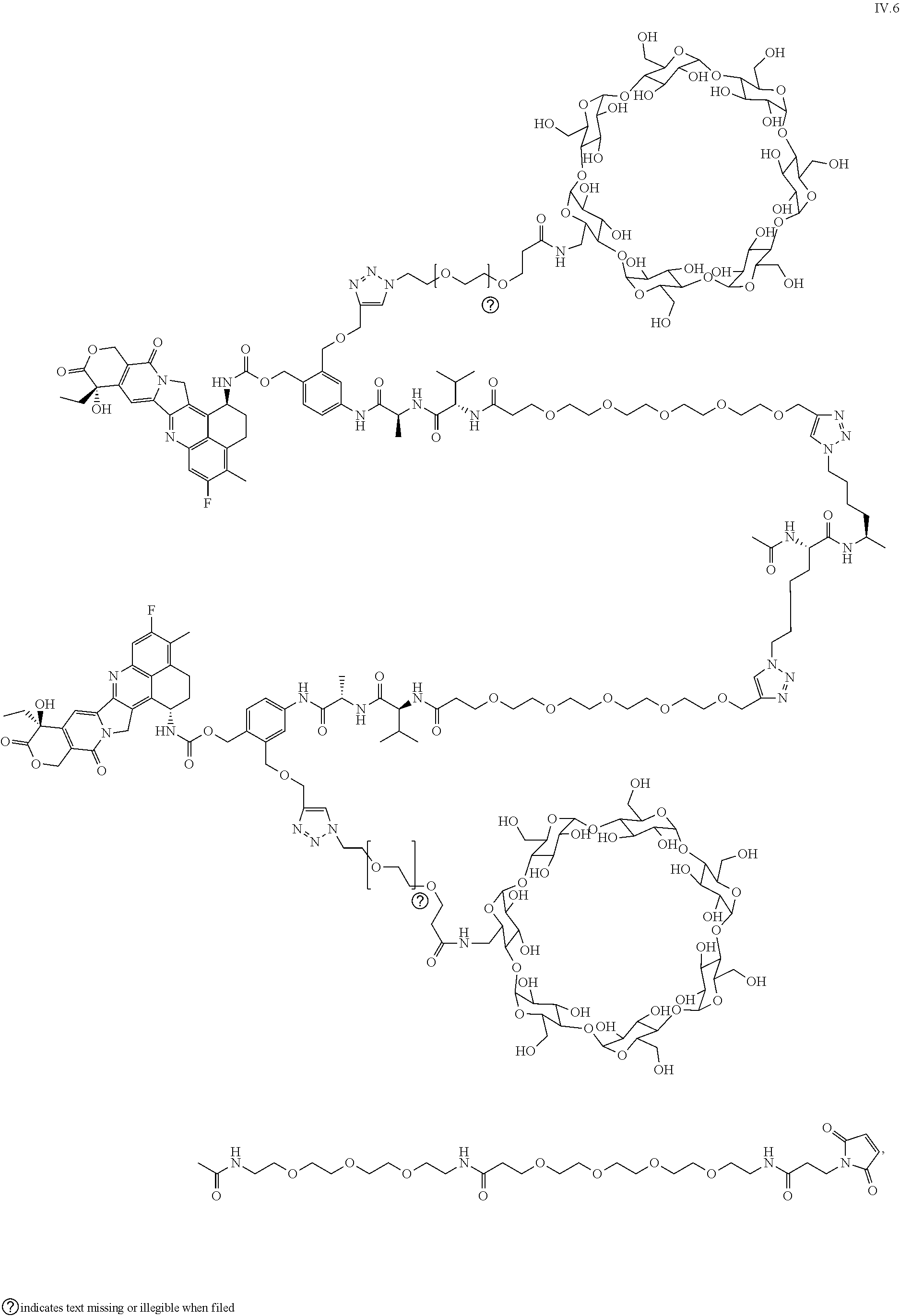

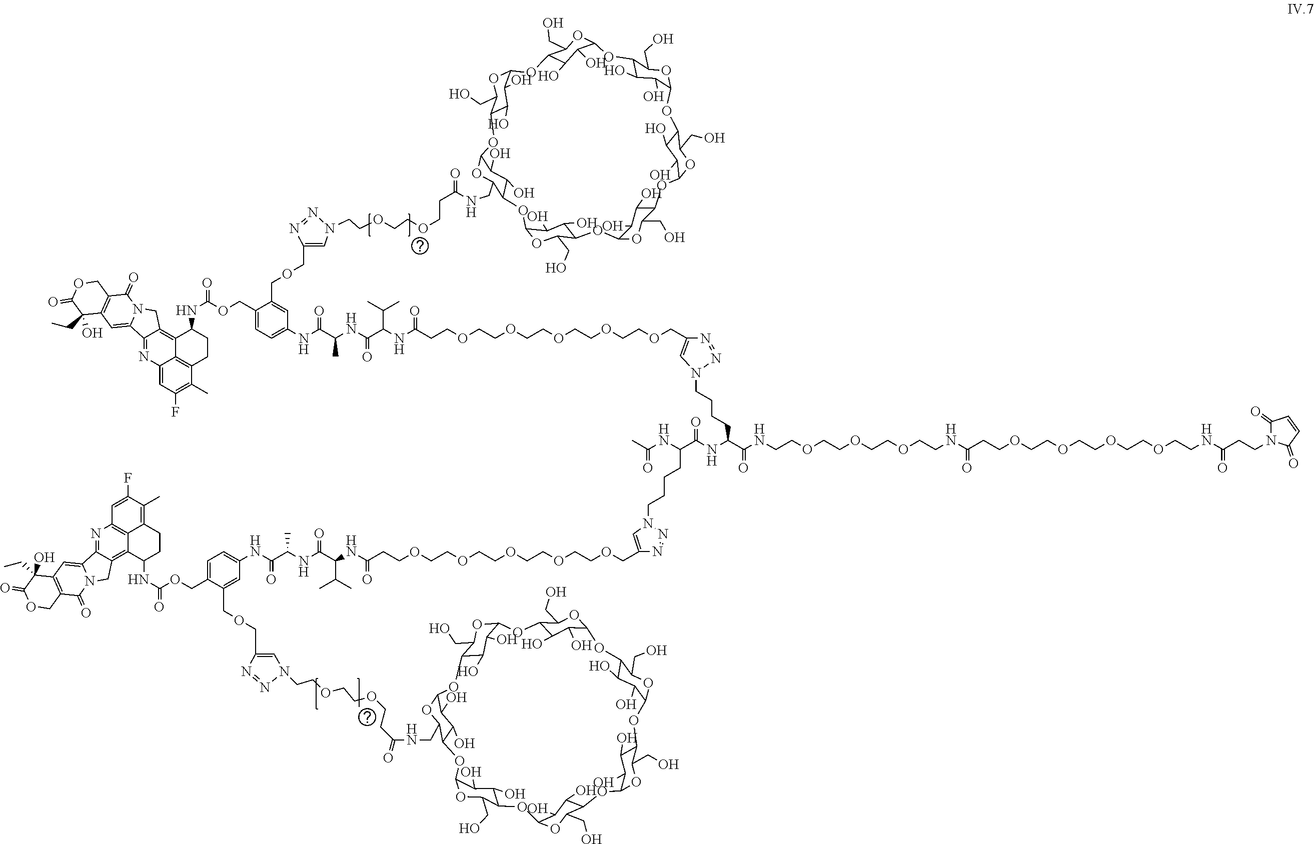

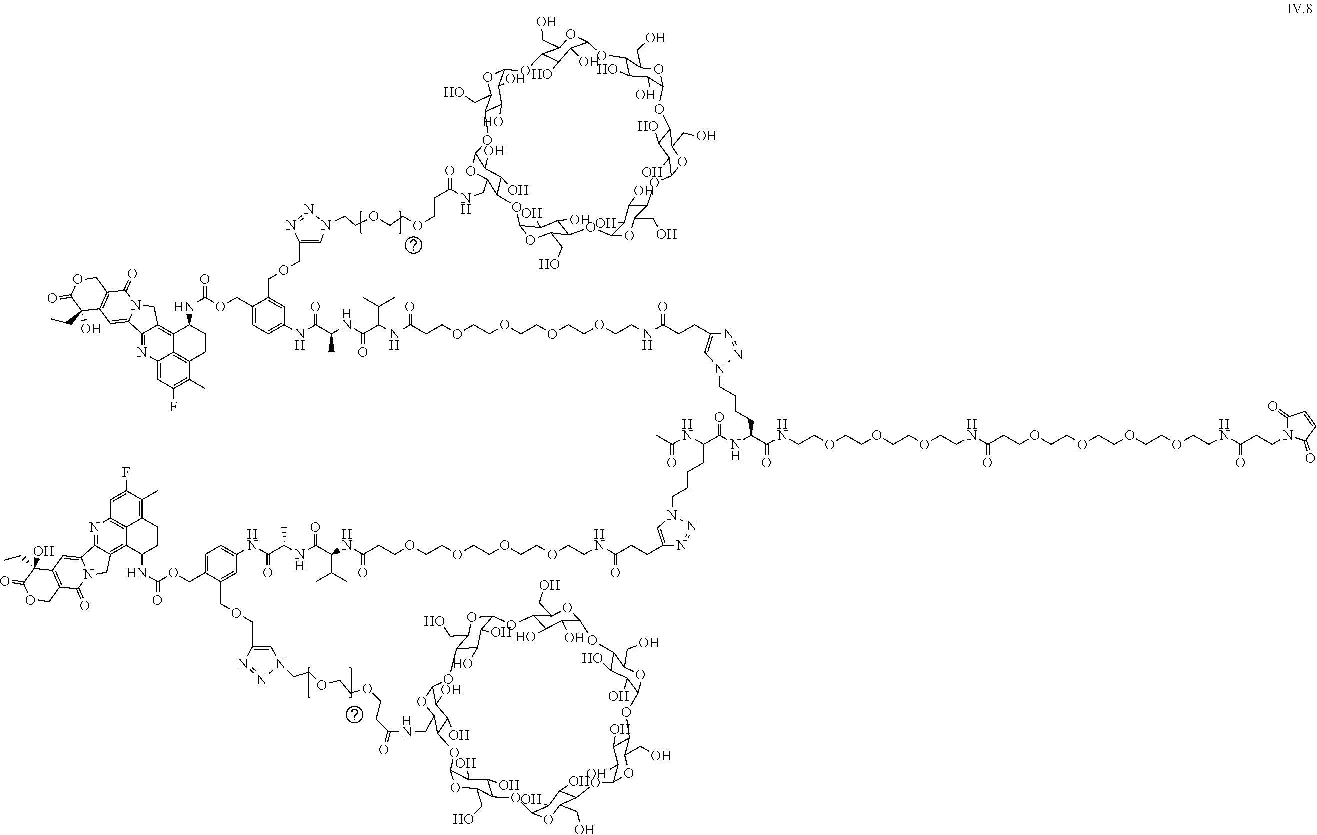

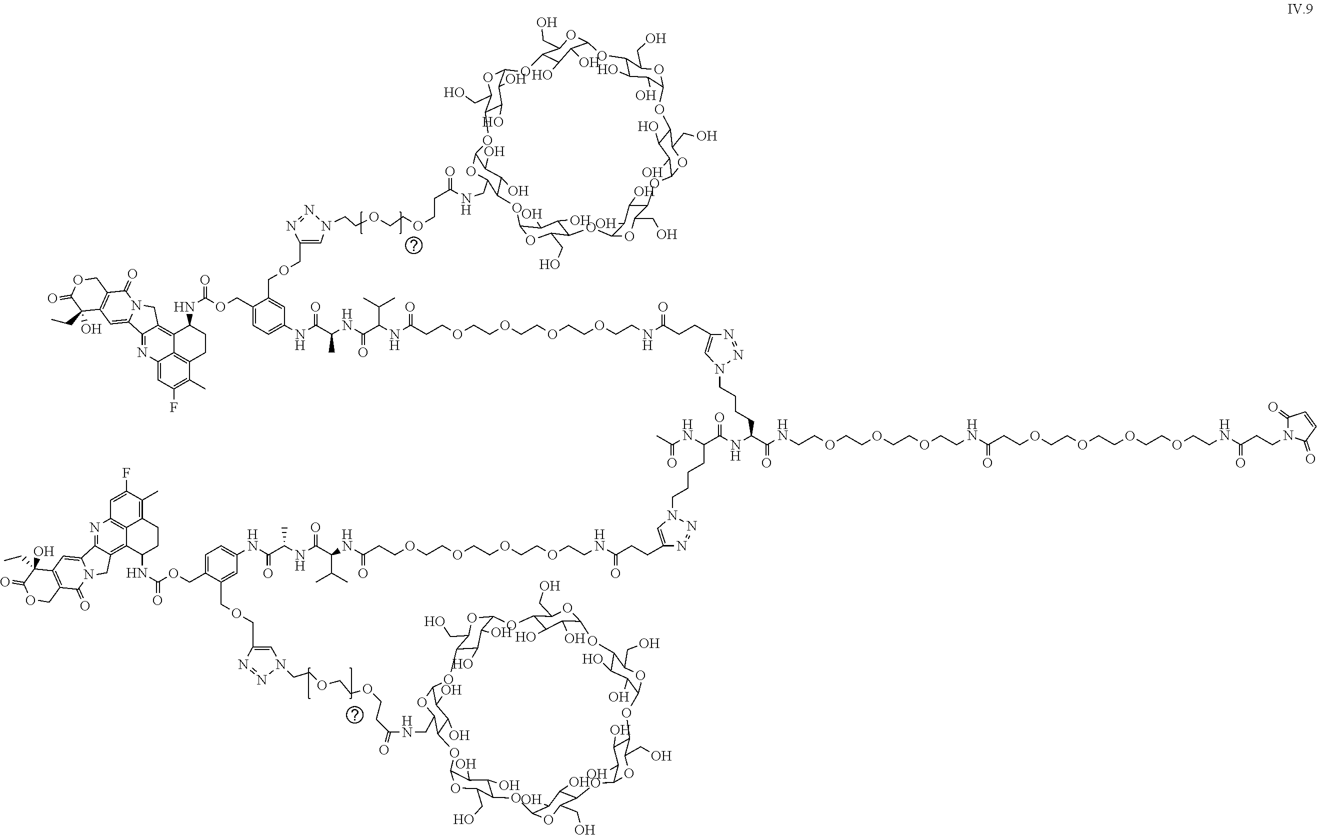

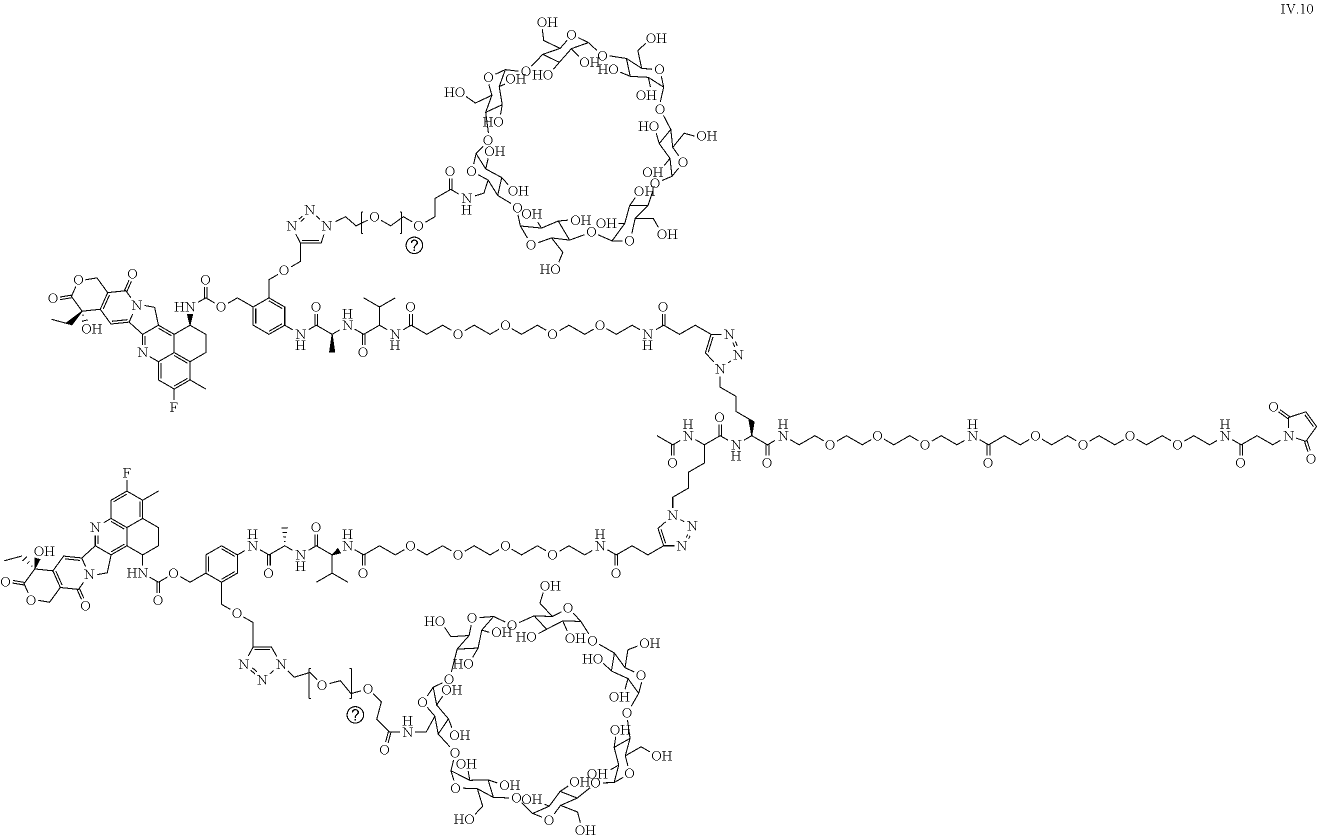

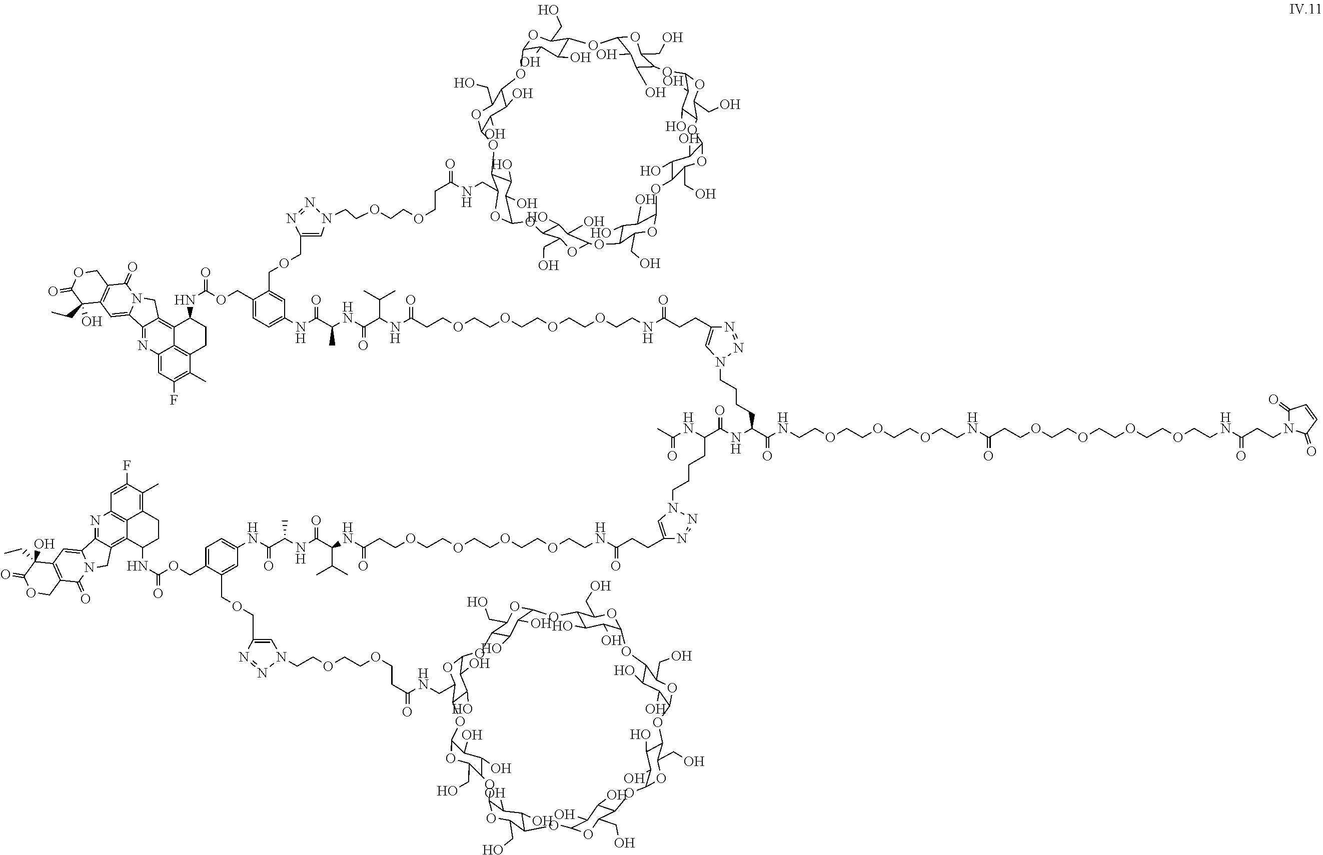

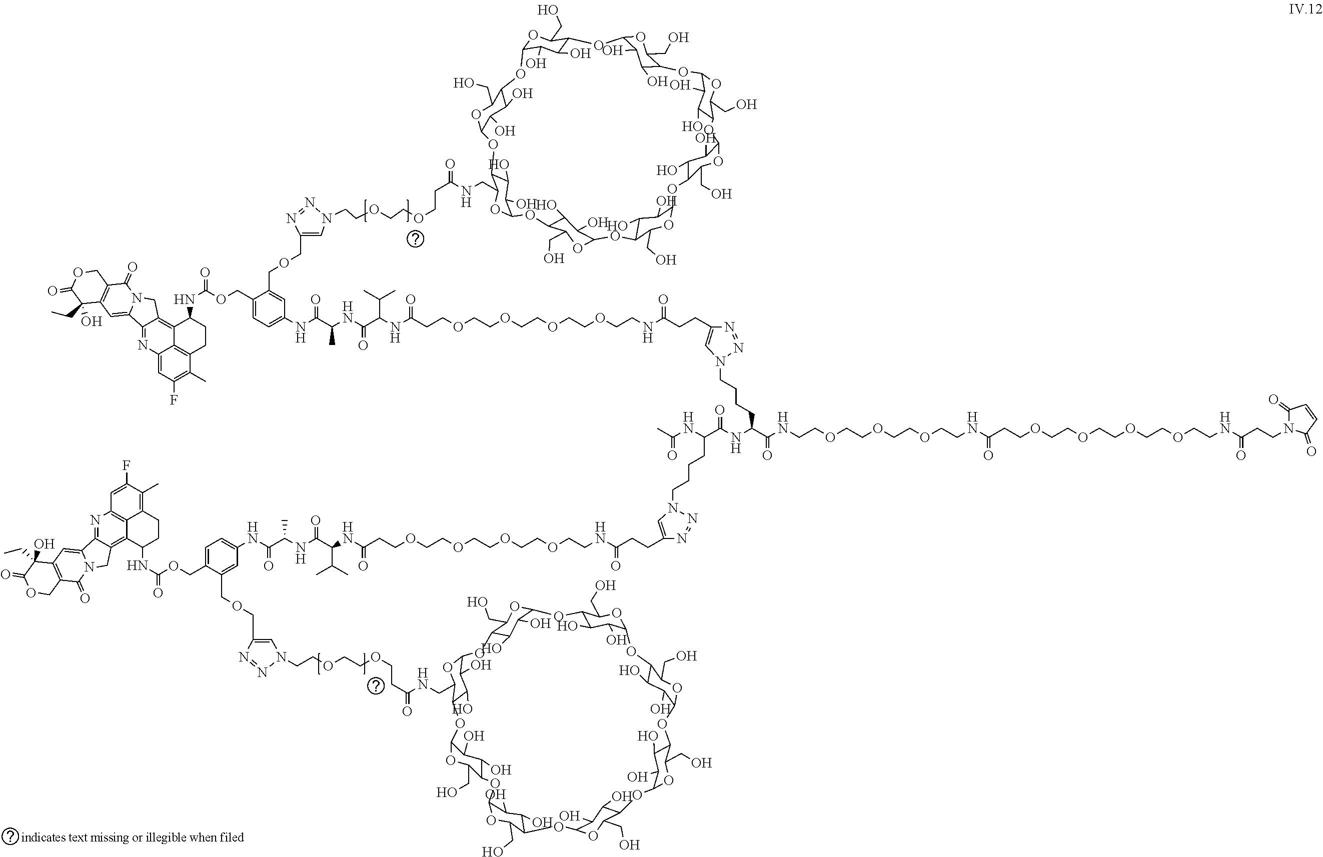

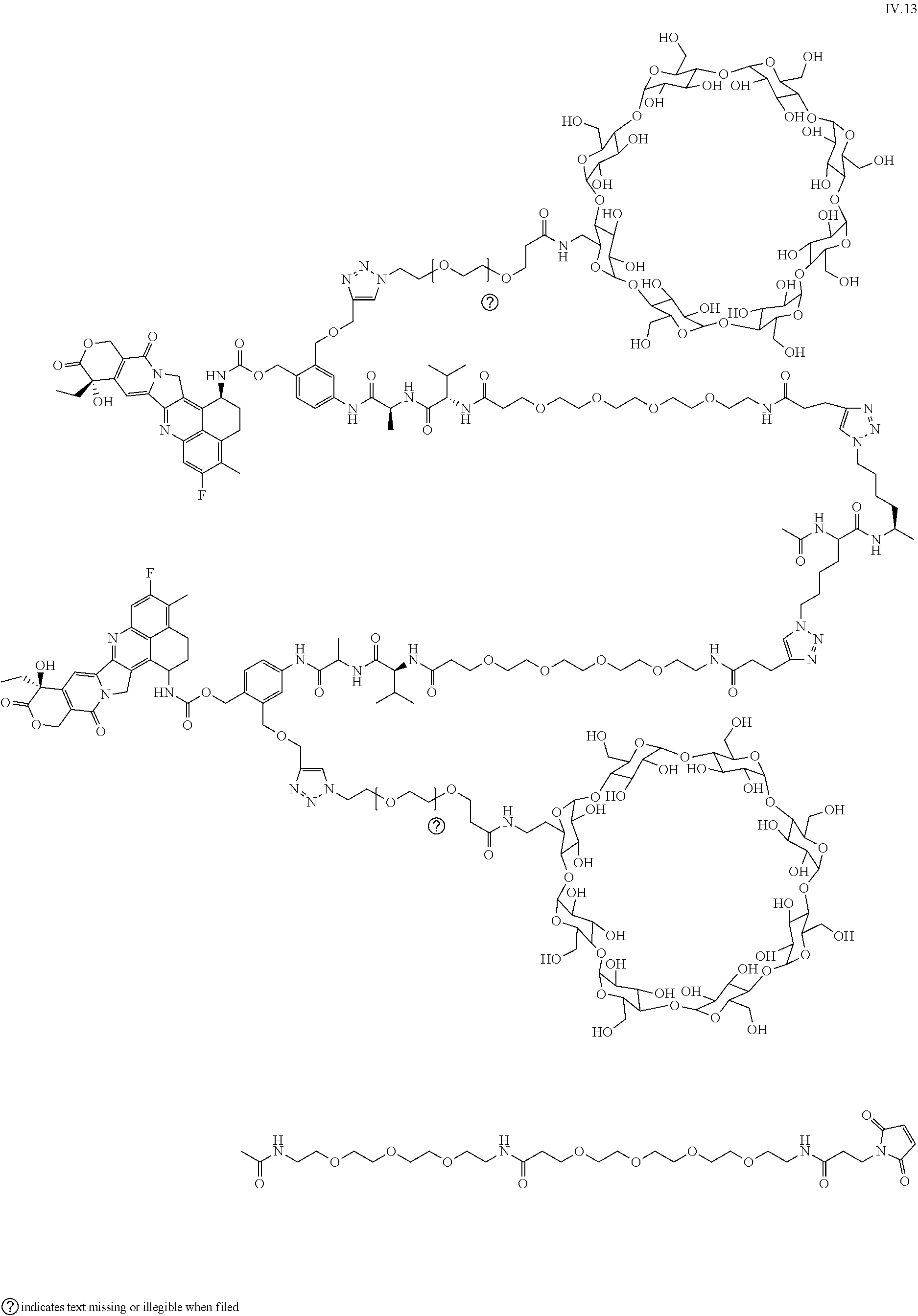

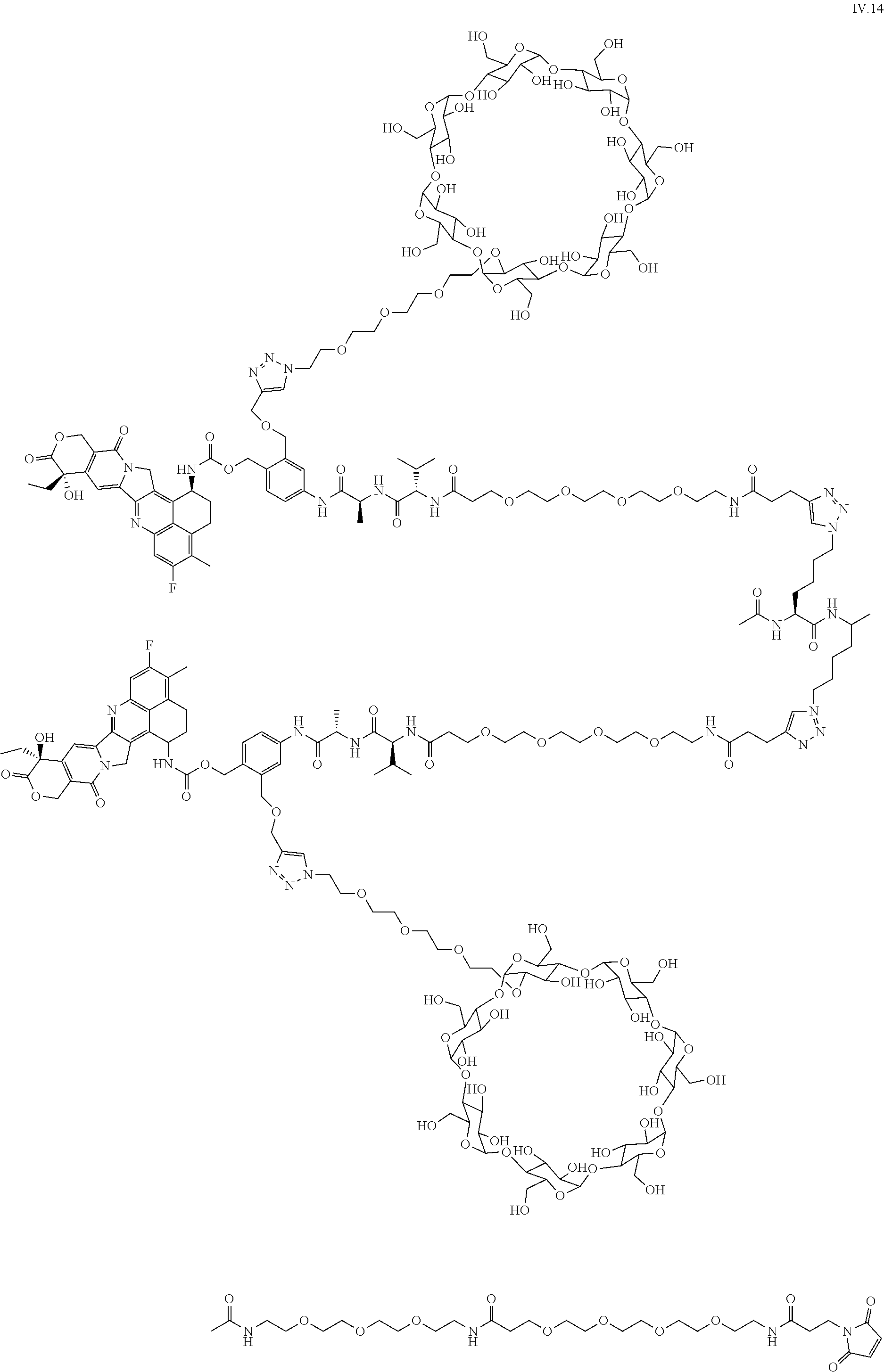

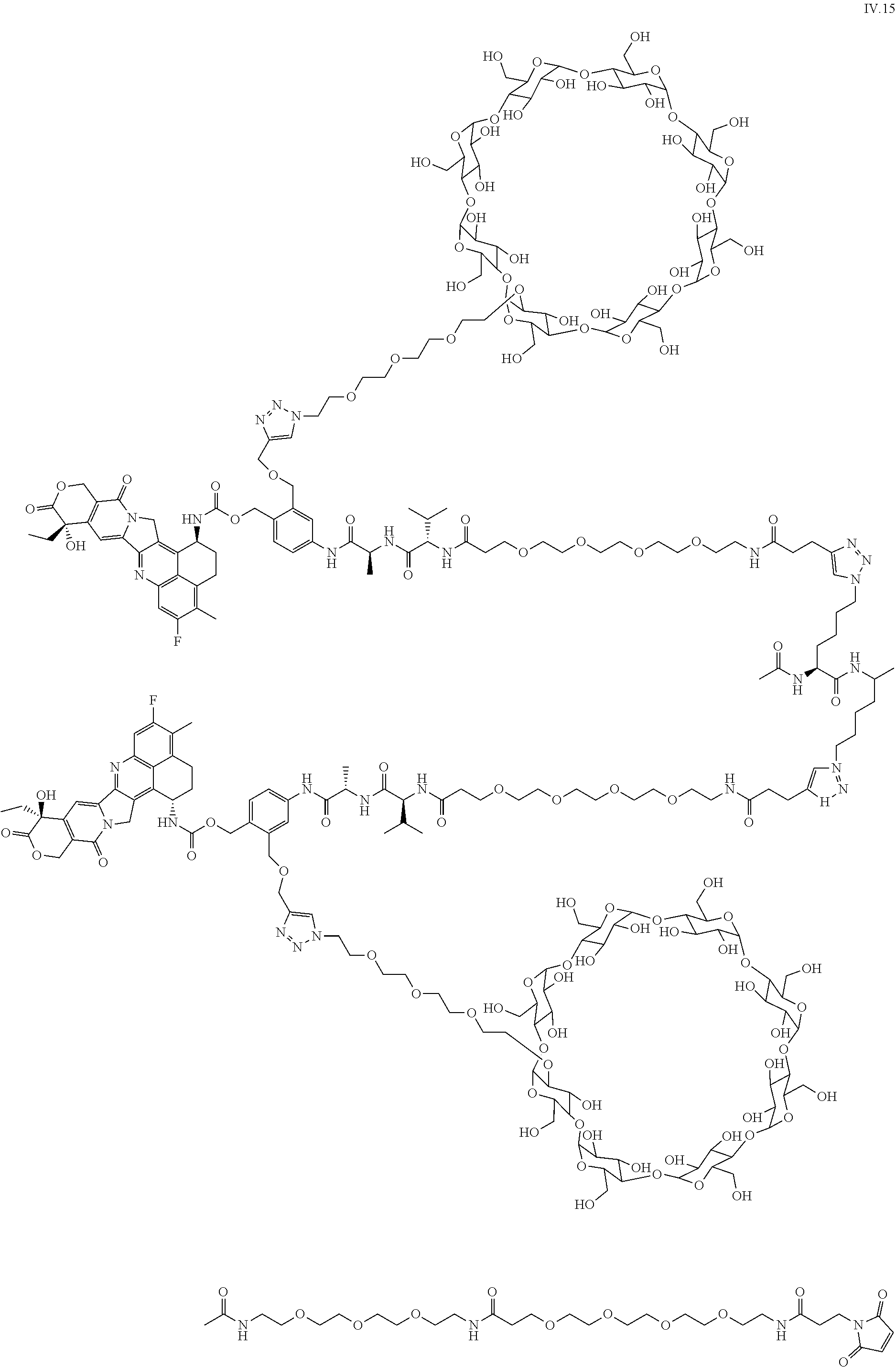

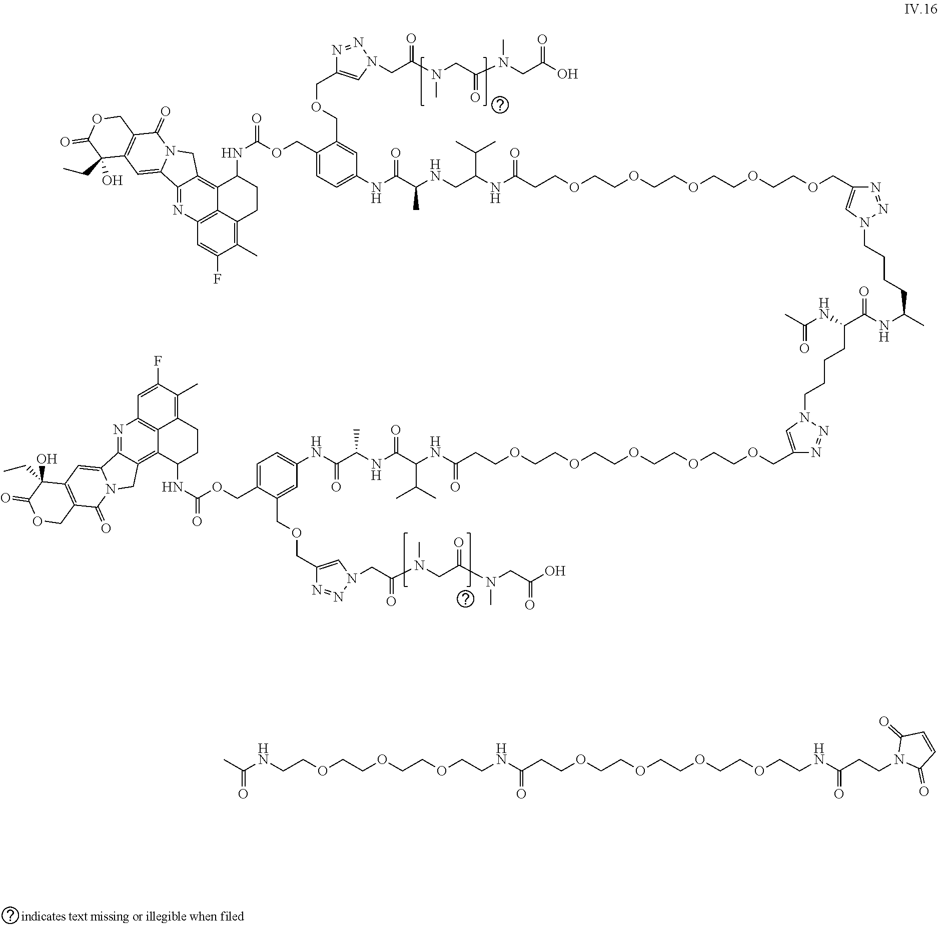

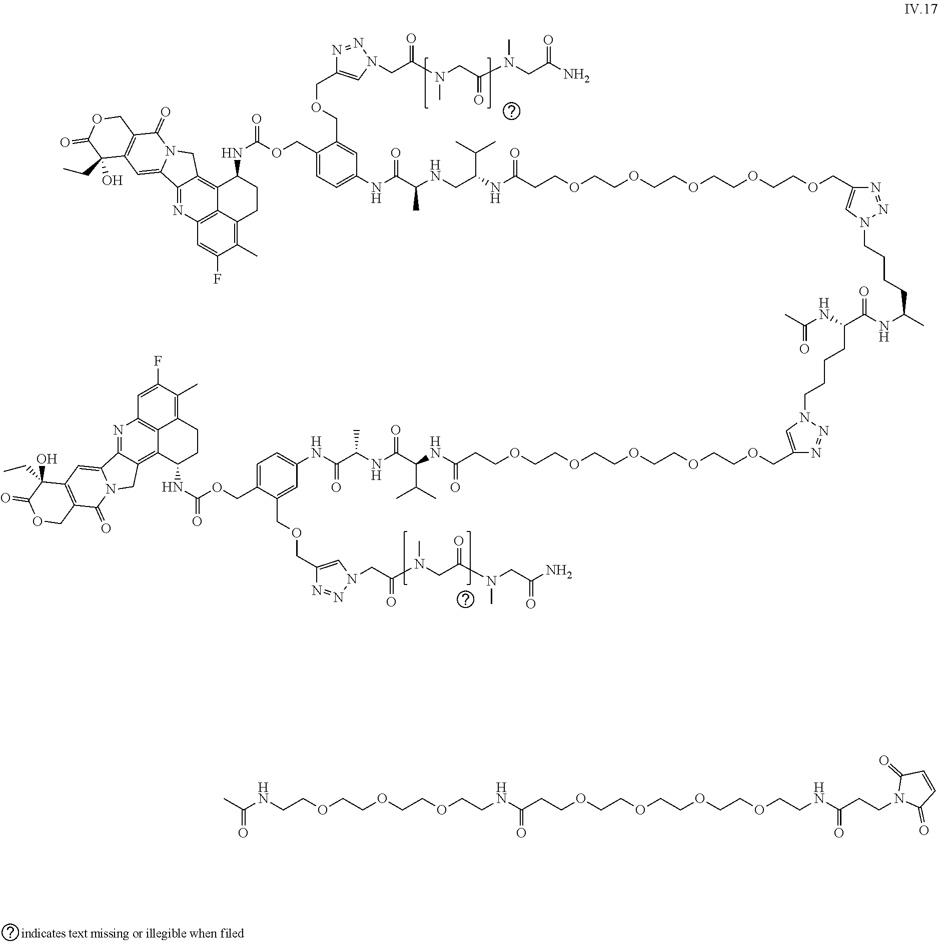

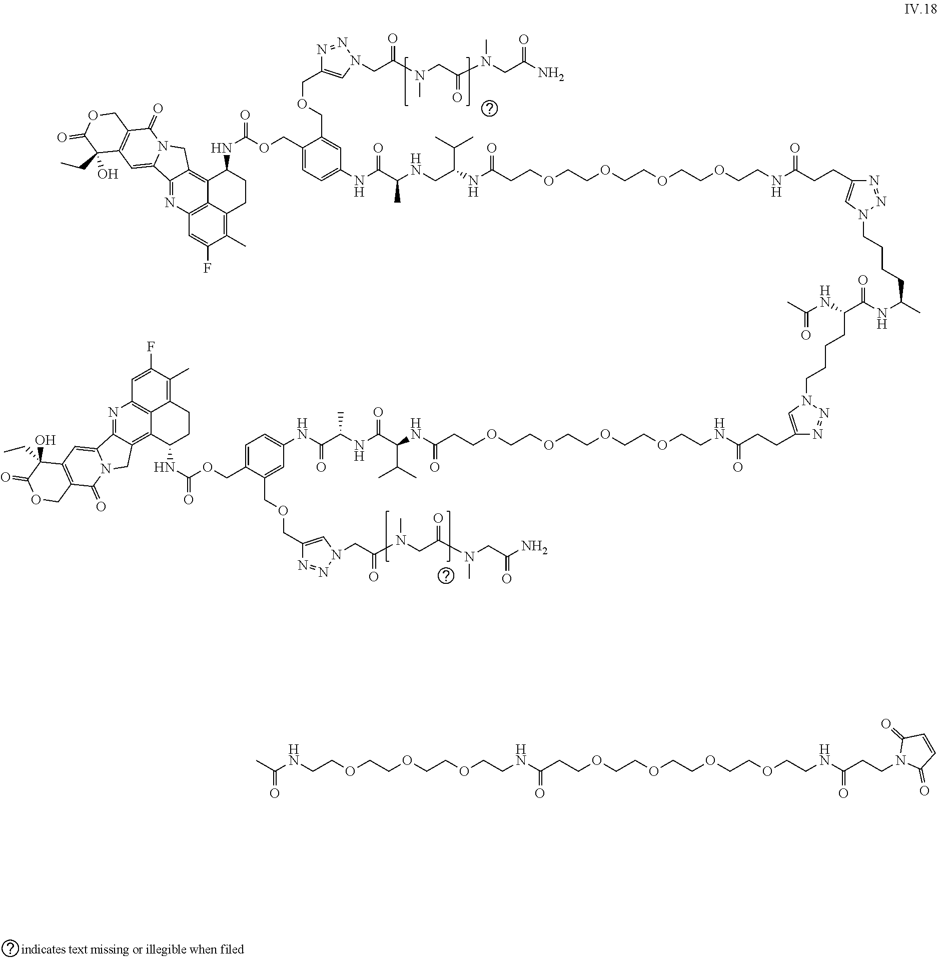

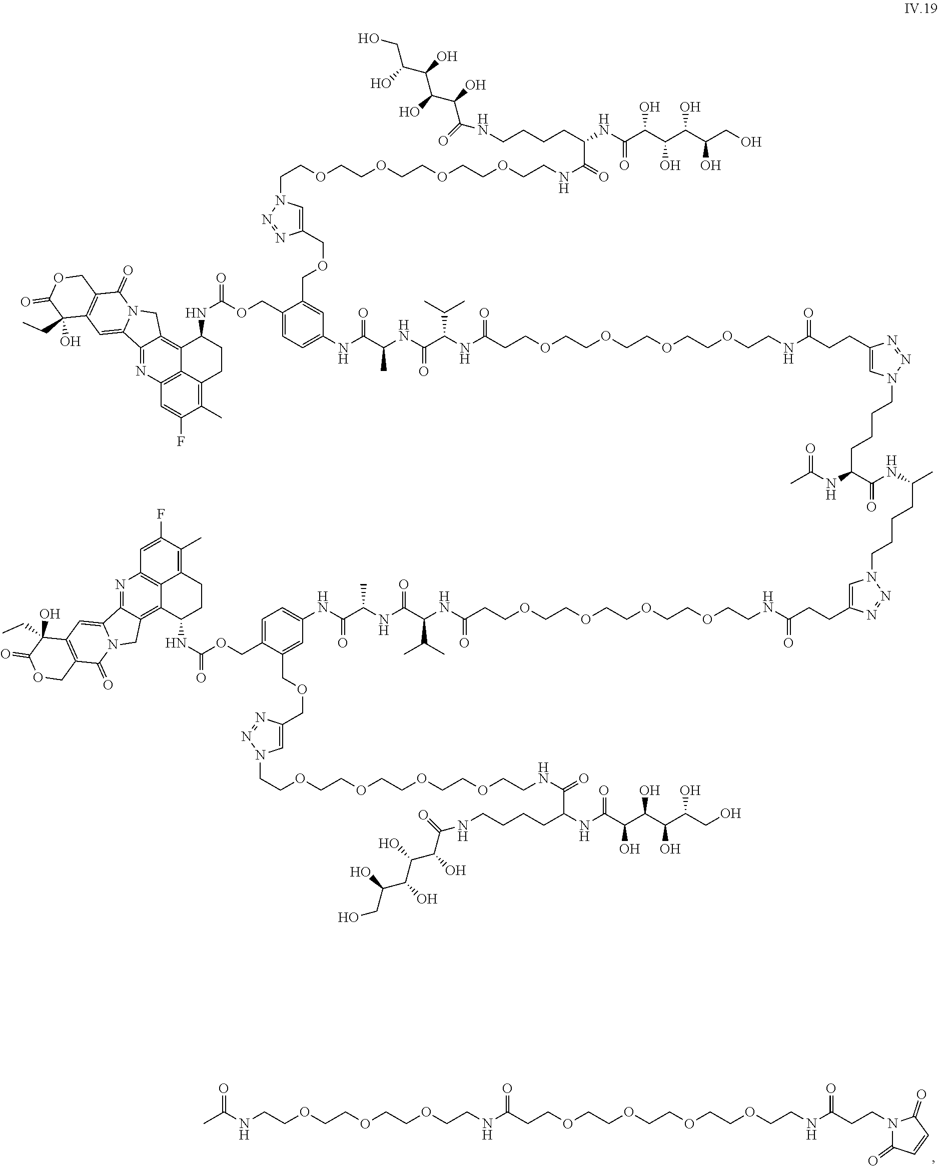

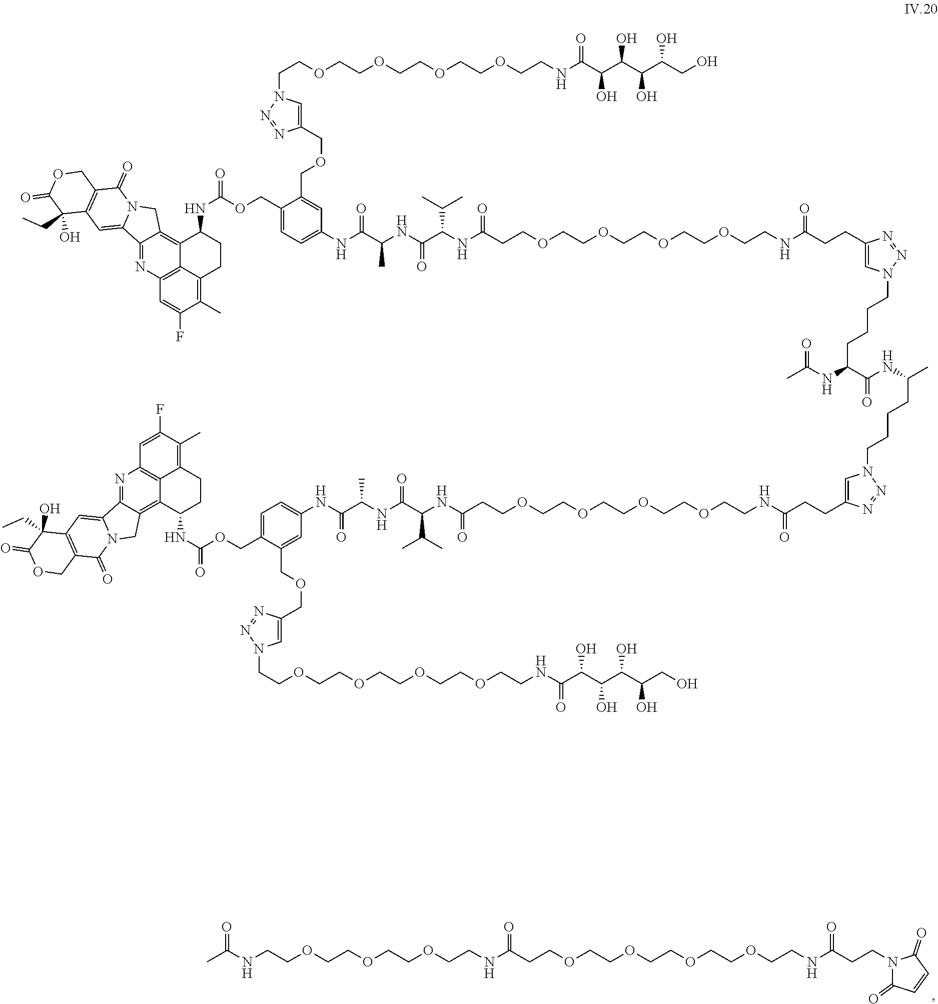

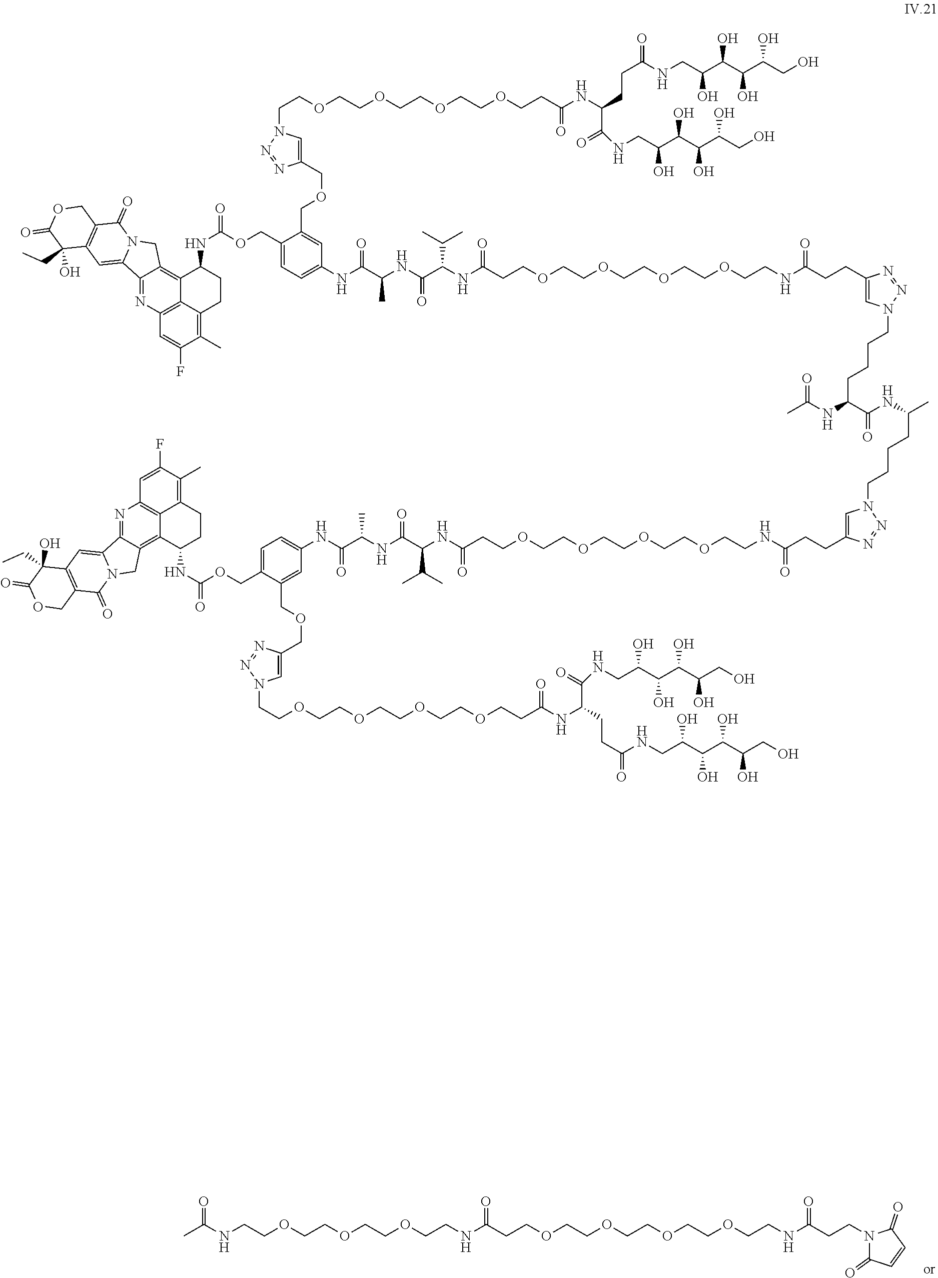

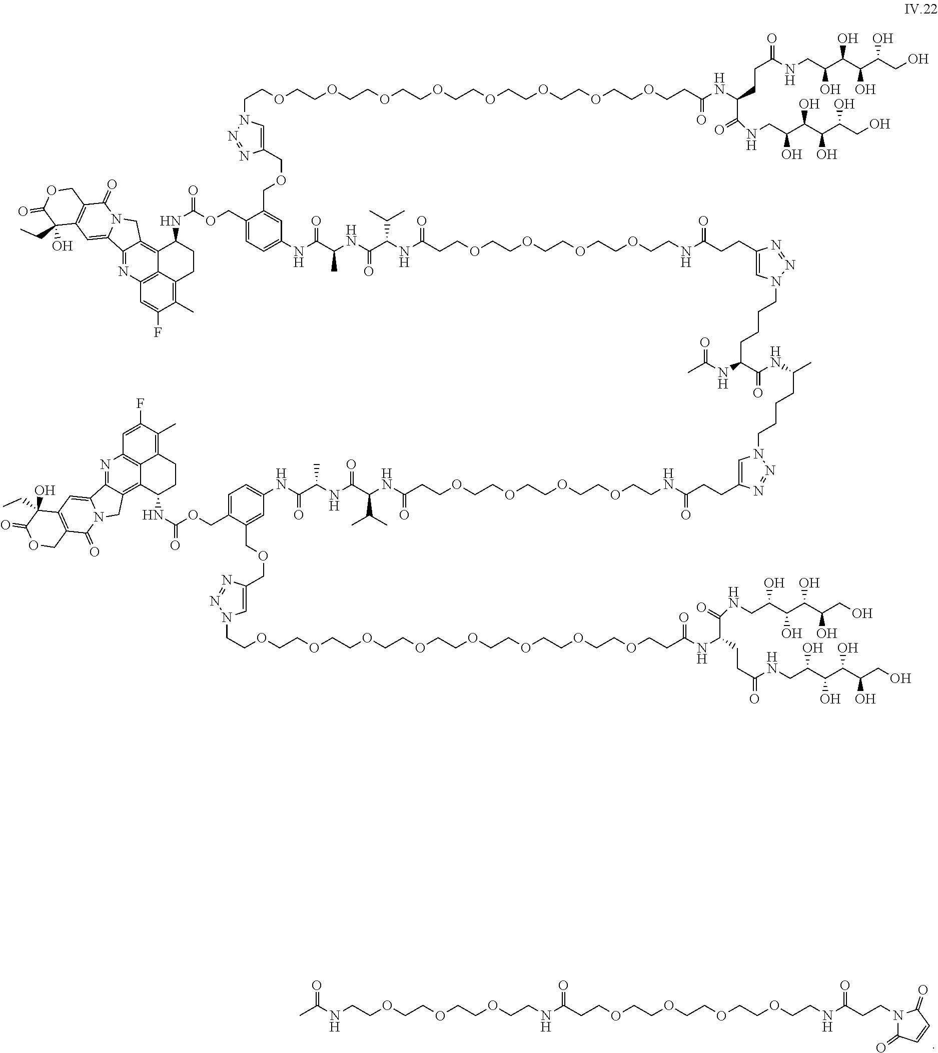

9. A method for synthesizing a modular linker compound of Formula (I) of claim 1, the method comprising:
(i) contacting a first compound comprising:
an immolative moiety,
a cleavable peptide sequence W,
an amine protecting group Pg¹, which is covalently linked to W,
a pharmaceutically active substance D and
a first complementary group Cg¹,
with a second compound comprising:
a solubility enhancing group SE and
a second complementary group Cg²
to form an intermediate of Formula II: 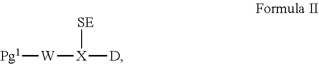
wherein SE is connected to X via R² and wherein R² is formed by the reaction of Cg¹ and Cg²;
(ii) removing the amine protecting group Pg¹ from Formula II to expose a primary amine;
(iii) contacting said primary amine with a third compound comprising
a spacer Z³,
an amine reactive group, and
a third complementary group Cg³
to form the intermediate of Formula III: 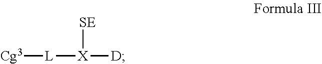
wherein L is 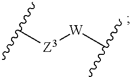
(iv) contacting the intermediate of Formula III with a branched fourth compound comprising
a fourth complementary group Cg⁴ and
a fifth complementary group Cg⁵,
wherein Cg³ reacts with Cg⁴ to form Z²; and
(v) contacting the product of (iv) with a fifth compound comprising
a reactive moiety A for conjugation to an target-binding moiety and
a sixth complementary group Cg⁶
wherein reaction of Cg⁵ and Cg⁶ forms Z¹, to form the modular linker compound of Formula I:

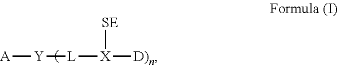

10. The method of claim 9, wherein:
X is a self-immolative spacer 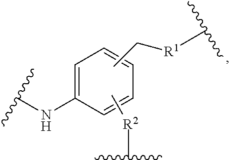
wherein:
R¹ covalently links X to D in para or ortho to the amino group of X and is a bond or 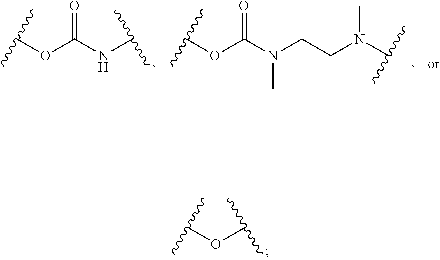
and
R² connects X to SE and is

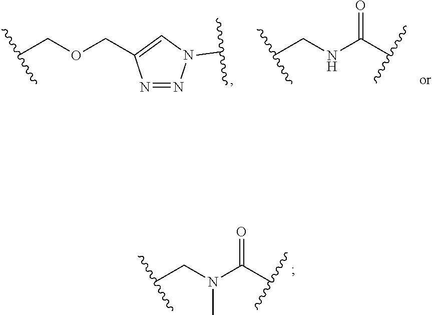

11. The method of claim 9,
wherein W is a cleavable or non-cleavable peptide sequence selected from the group consisting of:
Val-Ala, Val-Cit, Met-Thr, Thr-Thr, Phe-Lys, Glu-Val-Ala, Glu-Val-Cit, Gly-Gly-Phe-Gly, Gly-Gly-Tyr-Gly, cBu-Ala, cBu-Cit, Glu-cBu-Ala, Glu-cBu-Cit, Asp-cBu-Ala, Asp-cBu-Cit, Asp-Pro-Val, Asn-Pro-Val, iGlu-cBu-Ala, iGlu-cBu-Cit, Val-Lys, Val-Arg, Val-Gin, Val-Met, Val-Thr, AcLys-Val-Cit, AcLys-Val-Ala, Asp-Val-Ala, iGlu-Val-Ala, Asp-Val-Cit, iGlu-Val-Cit, Tyr-Arg, Tyr-Met, Phe-Cit, Phe-Ala, Phe-Arg, Phe-Met, Phe-Gin, Phe-Arg-Arg-Gly, Phe-Arg-Arg-Leu, Phe-Arg-Arg-Leu-Gly, Phe-Leu-Arg-Arg-Gly, Ala-Lys, Leu-Cit, Leu-Gln, Ile-Cit, Ala-Ala-Asn, Ala-Asn, Ala-Ala, Asn-Asn, Asn, beta-Ala, and Lys or combinations thereof; and
wherein Z³ is a bond or a spacer comprising a C₁₋₂₀ alkyl chain or a 1 to 10 polyethylene glycol (PEG)₁₋₁₀ or a C₂-amide-PEG₁₋₁₀;
wherein L optionally is

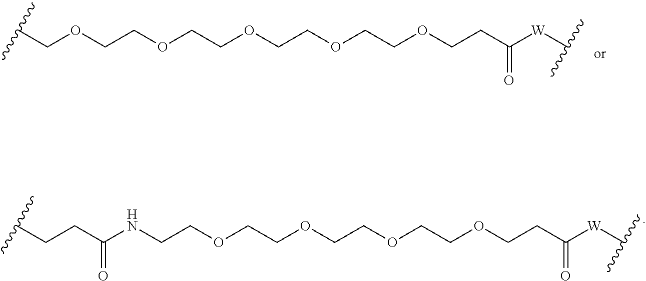

12. The method of claim 9, wherein
Y is: 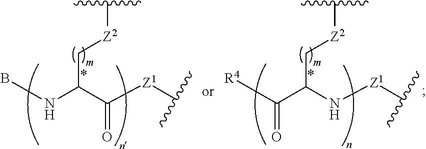
m is an integer from 1-6;
Z¹ connects Y to A and is 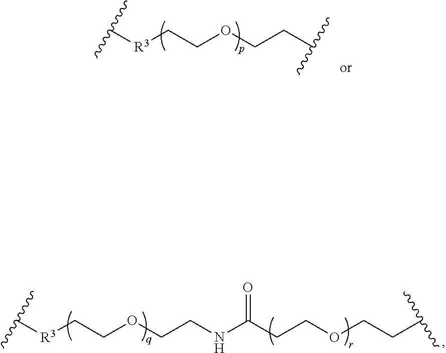
wherein,
R³ is —O—, —NH—, —C(═O)—, —C(═O)N(CH₃)—, —C(═O) NH—, —NHC(═O)—, or —N(CH₃) C(═O)—, and
p is an integer from 1 to 24, optionally from 4 to 24, optionally from 8 to 24,
q is an integer from 1 to 8,
r is an integer from 1 to 24,
Z² connects Y to Land is selected from the group consisting of: 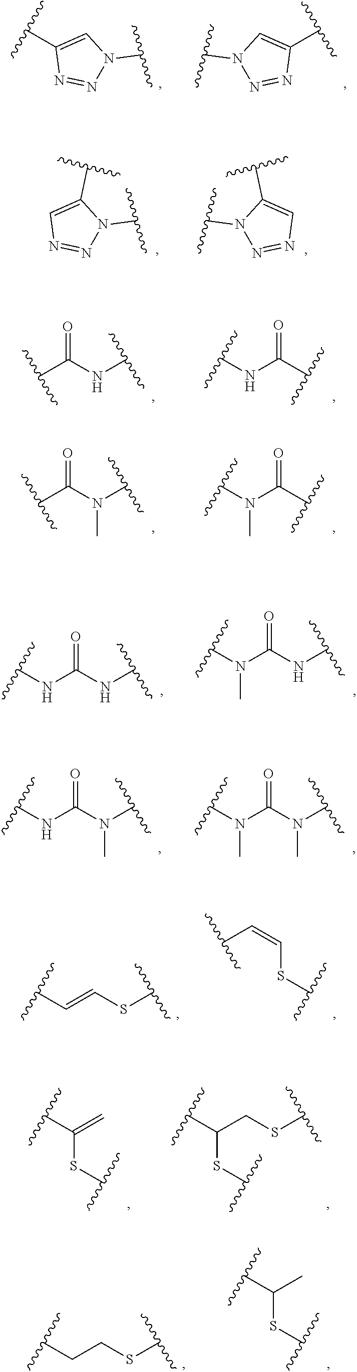 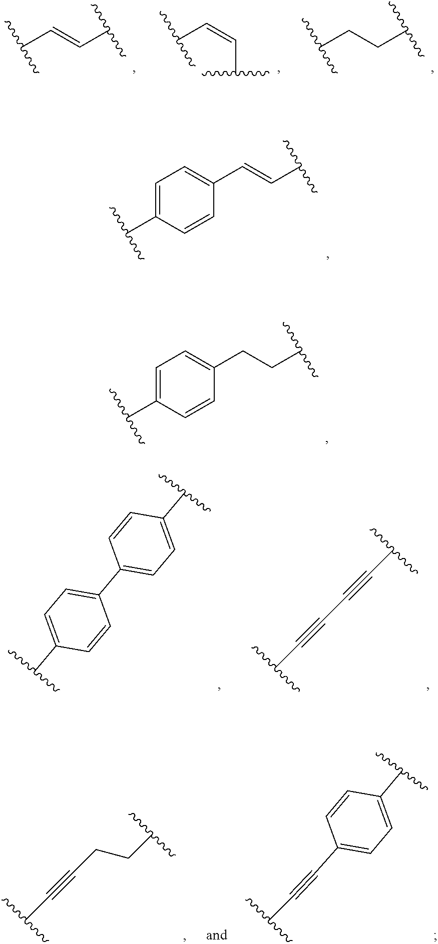
B is:
a cap, optionally an acyl group, and n′ is n; or 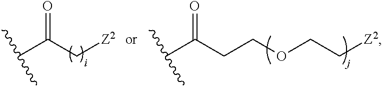
wherein
i is an integer from 1 to 20,
j is an integer from 1 to 10, and
n′ is n 1; or 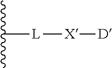
wherein,
D′ is a pharmaceutically active substance different from D;
X′ is self-immolative spacer 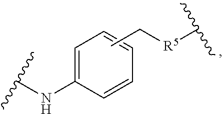
wherein:
R⁵ covalently links X′ to D′ in para or ortho to the amino group of X′ and is a bond, 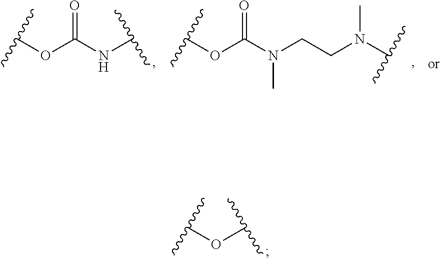
and n′ is n; and
R⁴ is OH or NH₂.

13. The method of claim 9, wherein
Y is: 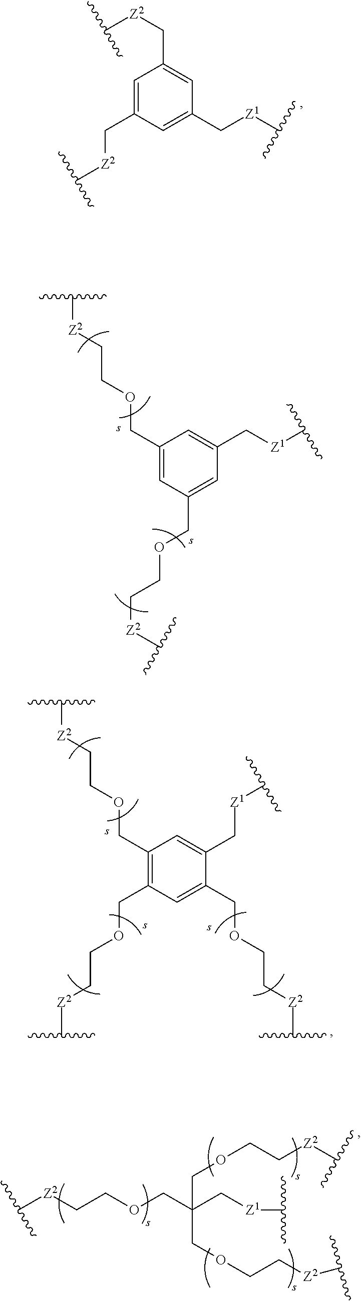 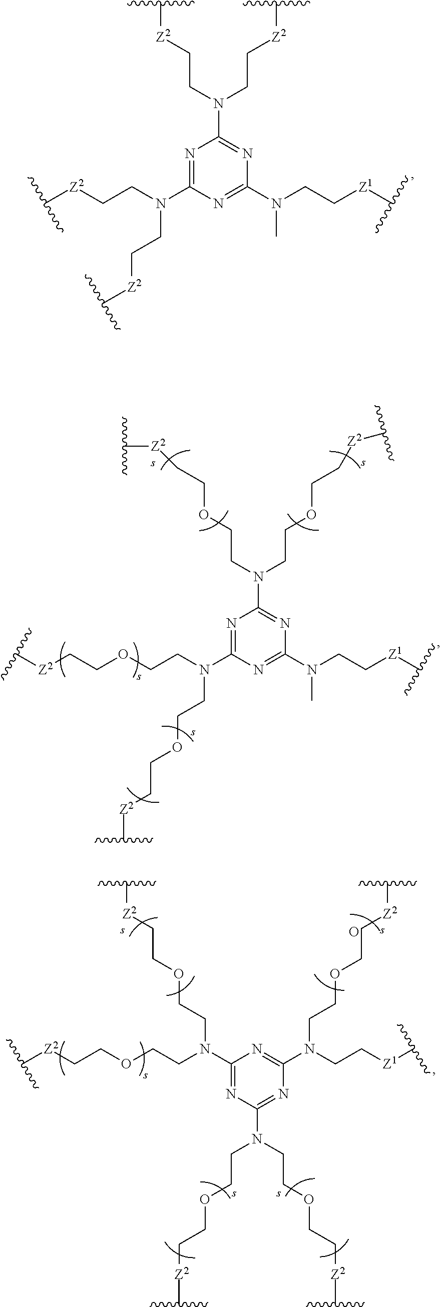 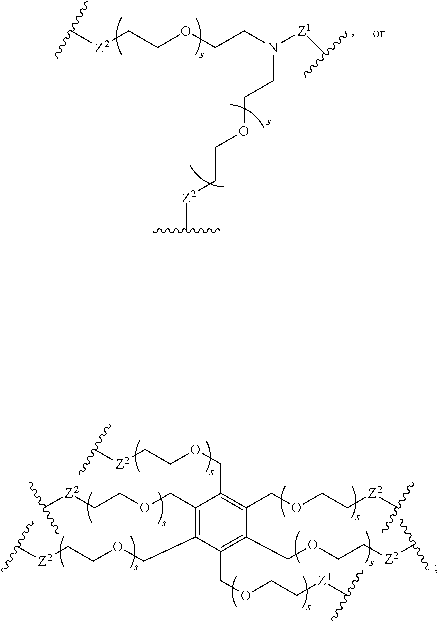
Z¹ connects Y to A and is 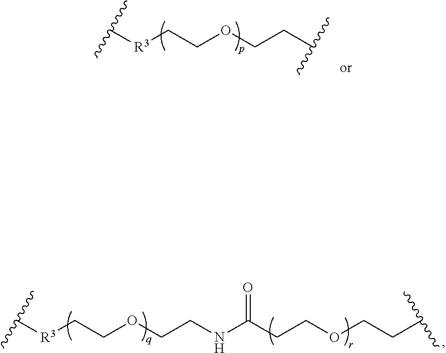
wherein
R³ is —O—, —NH—, —C(═O)—, —C(═O)N(CH₃)—, —C(═O) NH—, —NHC(═O)—, or —N(CHs) C(═O)—,
p is an integer from 1 to 24,
q is an integer from 1 to 8,
r is an integer from 1 to 24,
s is an integer from 0 to 4,
Z² connects Y to Land is selected from the group consisting of:

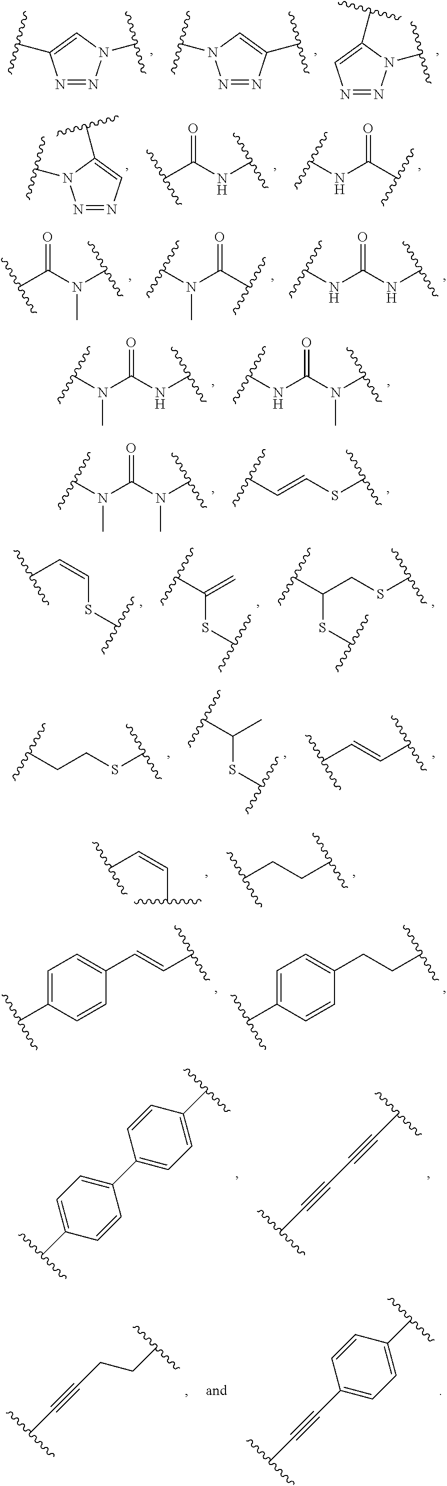

14. The method of claim 9, wherein D and D′ are independently selected from the group consisting of camptothecins, optionally exatecan, SN38 and topotecan; auristatins, optionally selected from monomethyl auristatin E (MMAE) and monomethyl auristatin F (MMAF); maytansinoids, optionally selected from mertansine (DM1) and ravtansine (DM4); calicheamicins; toll-like receptor 7 (TLR7) agonists; nicotinamide phosphoribosyltransferase (NAMPT) inhibitor (NAMPT); pyrrolobenzodiazepines (PBDs); duocarmycins; anthracyclines, optionally doxorubicin; amatoxins, optionally alpha-amanitin, beta amanitin, amanin and amaninamide; cryptophycins; phalloidins; seco-cyclopropylpyrroloindoline (seco-CPI); seco-cyclopropylbenzoindoline (seco-CBI); taxols; vinblastines; colchicines; eribulins; methotrexate (MTX); triptolide; and derivatives thereof.

15. The method of claim 9, wherein S comprises an alpha-cyclodextrin, a beta-cyclodextrin, a gamma-cyclodextrin, a polysarcosine, a PEG, a galactoside, a glucuronide or a polyalcohol made from carbohydrates, optionally 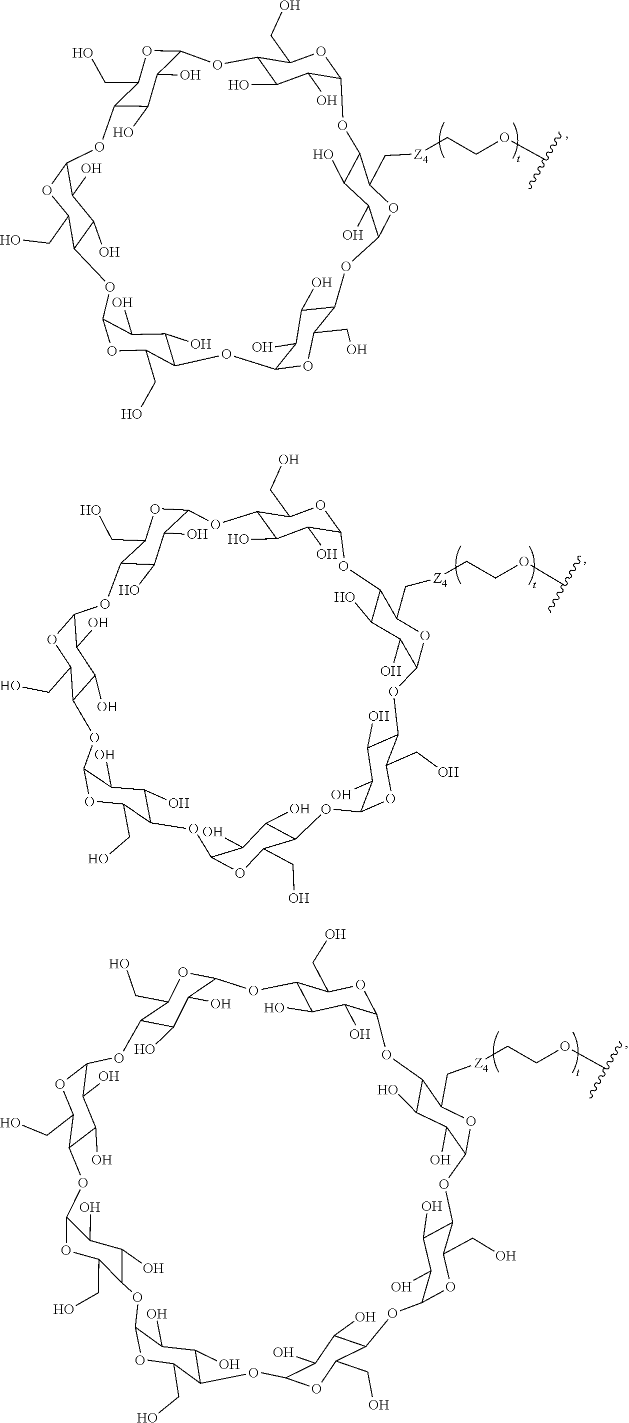 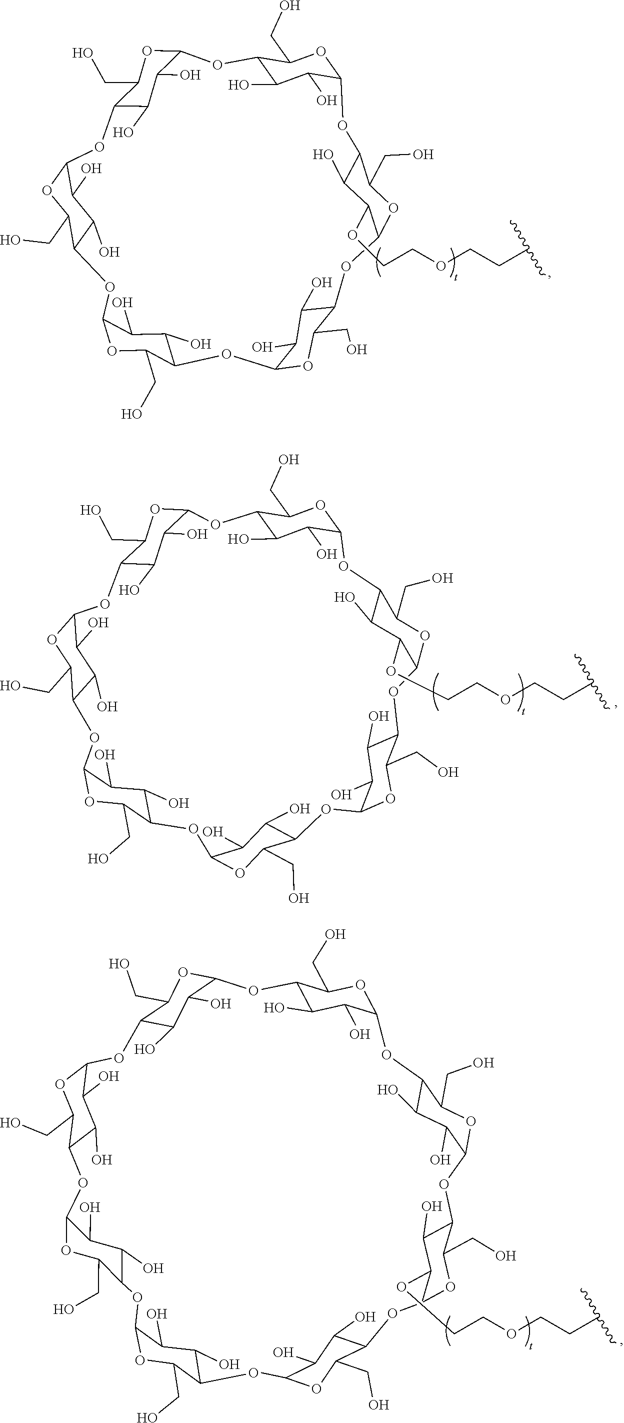 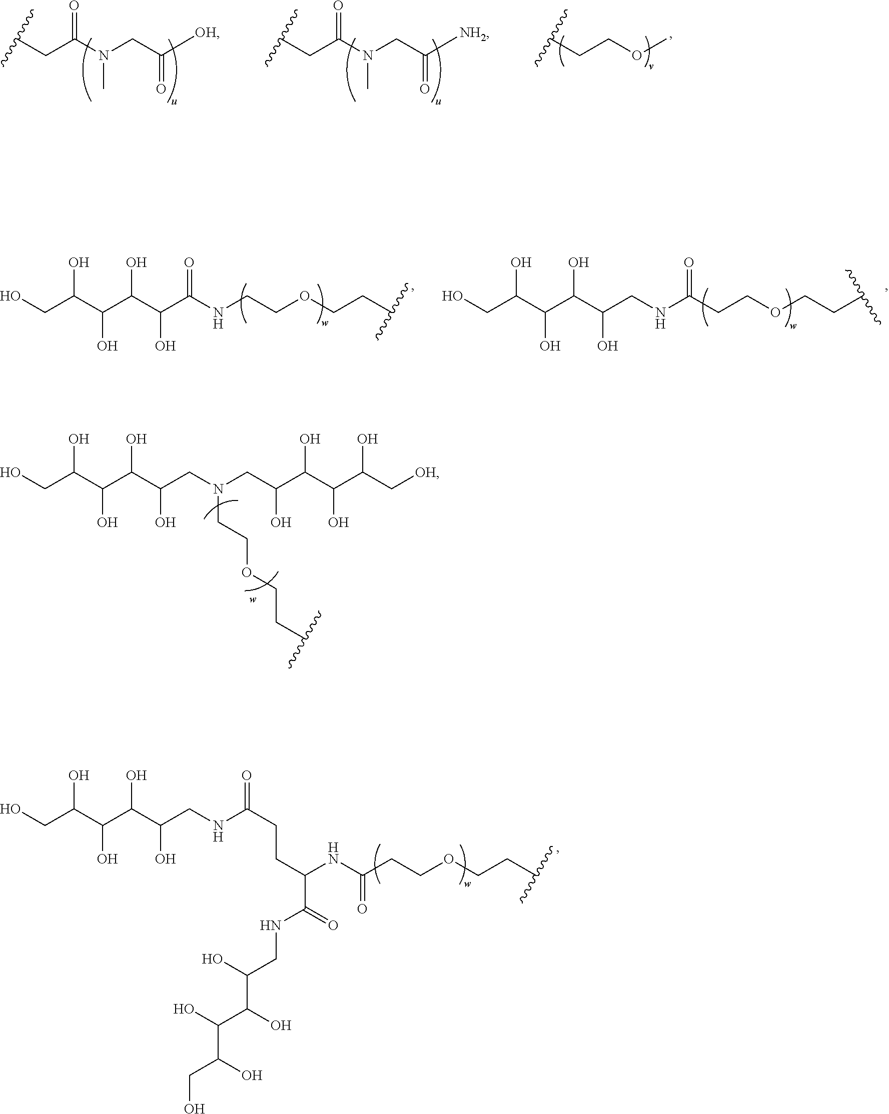 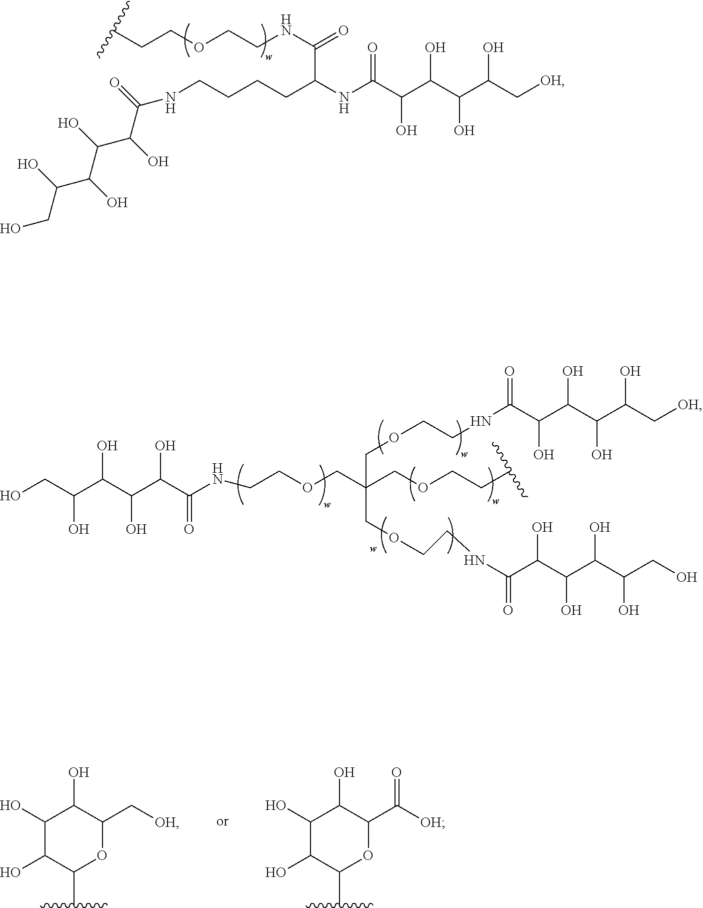
wherein Z⁴ is selected from the group consisting of: 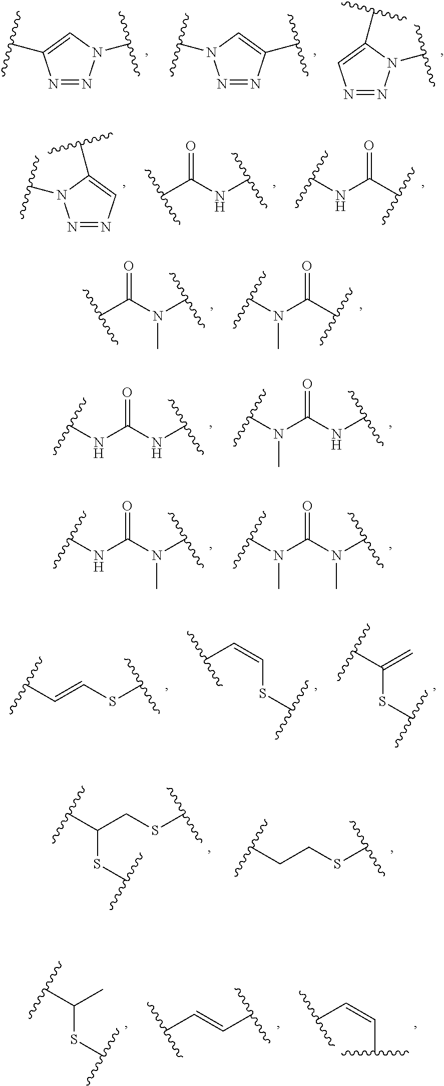 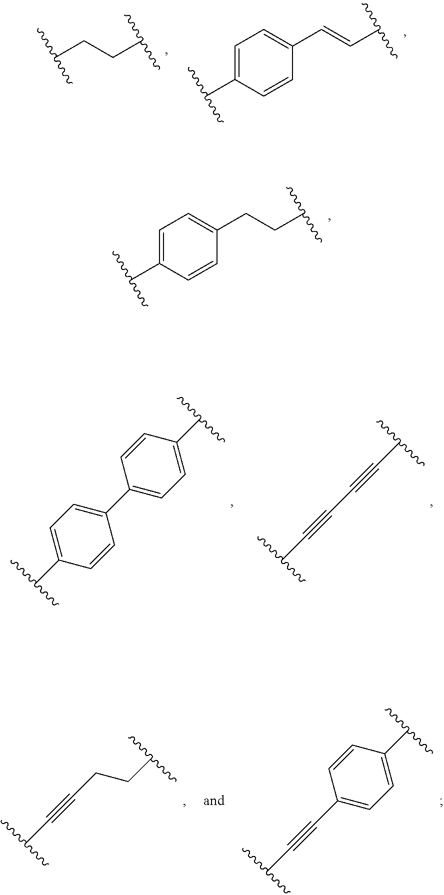
t is 2 to 18, optionally 8 to 12,
u is at least 1, optionally 10;
v is 4 to 24,
w is 2 to 24.

16. The method of claim 9, wherein
A is

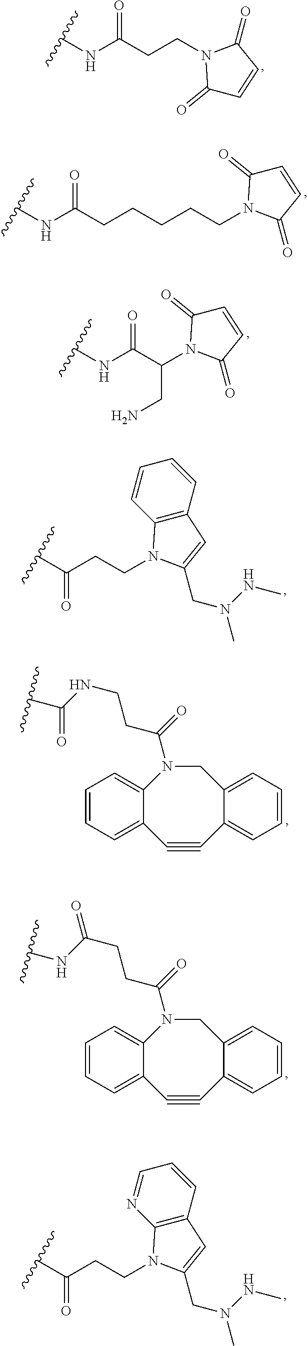

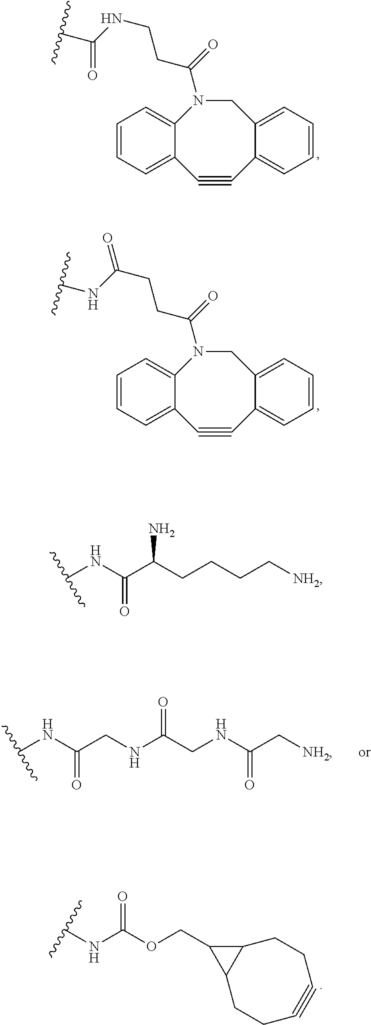 [SMILES: *C(=O)NCCC(=O)N1Cc2ccccc2C#Cc2ccccc21 ; *NC(=O)CCC(=O)N1Cc2ccccc2C#Cc2ccccc21 ; *NC(=O)[C@@H](N)CCCCN ; *NC(=O)CNC(=O)CNC(=O)CN ; *NC(=O)OCC1C2CCC#CCCC21]

17. The method of claim 9, wherein
Cg¹ is an alkyne group and Cg² is an azide group, or
Cg¹ is a primary or secondary amine group and Cg² is a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group.

18. The method of claim 9, wherein
Cg³ is an alkyne group and Cg⁴ is an azide group;
Cg² is an azide group and Cg⁴ is an alkyne group;
Cg³ is an primary or secondary amine group and Cg⁴ is a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group; or
Cg³ is a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide, amide, carbamate, carbamoyl halide or urea group and Cg⁴ is a primary or secondary amine group.

19. The method of claim 9, wherein
Cg⁵ is a primary or secondary amine group and Cg⁶ is a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group; or
Cg⁵ is a carboxylic acid, carboxylic ester, optionally N-hydroxysuccinimide ester, tetrafluorophenyl ester, pentafluorophenyl ester, or N-hydroxyphtalimide ester, carboxylic anhydride, acyl halide or amide group and Cg⁶ is a primary or secondary amine group.

20. The method of claim 9, wherein the amine protecting group Pg¹ is a carbobenzyloxy (Cbz), tert-butyloxycarbonyl (Boc), 9-fluorenylmethyloxycarbonyl (Fmoc), acetyl (Ac), benzoyl (Bz), benzyl (Bn), p-bethoxybenzyl (PMB), p-toluenesulfonyl (Ts) or carbamate group.

21. A target-binding drug conjugate comprising at least one reaction product of the modular linker compound of claim 1 and a correspondingly reactive group of a target-binding moiety, wherein said at least one reaction product is the product of a reaction between A of the modular linker compound and the correspondingly reactive group of the target-binding moiety and covalently couples the modular linker compound to the target-binding moiety, optionally the correspondingly reactive group of the target-binding moiety is a thiol group.

22. The target-binding drug conjugate of claim 21 comprising more than one of said reaction products such that more than one modular linker compound, optionally between 2 to 10 such as 2, 3, 4, 5, 6, 7, 8, 9, or 10, is coupled to said target-binding moiety.

23. The target-binding drug conjugate of claim 21, wherein the target-binding moiety specifically recognizes and binds a target sequence or epitope of an antigen, optionally the target-binding moiety is:
an antibody such as a monoclonal antibody;
a functional antibody fragment or antibody derivative such as a single-chain variable fragment (scFv), an antigen-binding fragment such as a Fab fragment, a F(ab′), fragment, a F(ab′)2 fragment or a bi- or tri-specific antibody construct;
a Diabody,
a Camelid Antibody,
a Domain Antibody,
a Nanobody,
a bivalent homodimer with two chains consisting of scFvs,
a shark antibody,
an antibody consisting of new-world primate framework sequences plus non-new world primate complementarity-determining regions (CDR) or
a dimerised construct comprising a constant heavy chain 3 (CH₃) domain, a variable light chain (V_{L}) and a variable heavy chain (V_{H})
a TCR.

24. The target-binding drug conjugate of claim 21, wherein the target-binding moiety specifically recognizes and binds a target sequence or epitope of Guanylate Cyclase C (GUCY2C), Cluster of Differentiation 33 (CD33), Cluster of Differentiation 30 (CD30), Cluster of Differentiation (CD5), Cluster of Differentiation 20 (CD20), Cluster of Differentiation 45 (CD45), Cluster of Differentiation 117 (CD117/c-Kit), Cluster of Differentiation 123 (CD123/IL3RA), Cluster of Differentiation 137 (CD137/4-1BB), Cluster of Differentiation 269 (CD269/B-cell maturation antigen, BCMA), claudin 18.2, CEA (Carcinoembryonic antigen), Epithelial Cell Adhesion Molecule (EpCAM), DKK1 (Dickkopf-related protein 1), glypican-3, muscin-1 (MUC-1/CD227), Mucin 5AC (MUC5AC), Carbonic Anhydrase 9 (CAIX), C—X—C Motif Chemokine Receptor 1 (CXCR1, CD181), C—X—C Motif Chemokine Receptor 2 (CXCR2/CD182), human epidermal growth factor receptor 2 (HER2), Cluster of Differentiation 22 (CD22), Cluster of Differentiation 79b (CD79b), Nectin Cell Adhesion Molecule 4 (Nectin 4), Tumor-associated calcium signal transducer 2 (Trop-2), Cluster of Differentiation 19 (CD19), Delta-like 3 (DLL3), Transmembrane glycoprotein NMB (gpNMB), prostate-specific membrane antigen (PSMA), Hepatocyte growth factor receptor (c-Met, CD 151), Solute Carrier Family 39 Member 6 (LIV-1), Folate receptor 1 (FOLR1), Cluster of Differentiation 56 (CD56), Cluster of Differentiation 138 (CD138), Mesothelin, Carbonic anhydrase 6 (CA6), epidermal growth factor receptor (EGFR), Cluster of Differentiation 37 (CD37), or Ectonucleotide pyrophosphatase/phosphodiesterase family member 3 (ENPP3).

25. The target-binding drug conjugate of claim 21, wherein the target-binding moiety is any one of Adecatumumab (anti-EpCam), Amatuximab, (anti-mesothelin), Amivantamab (anti-EGFR), Besilesomab (anti-CEA), blinatumomab (anti-CD19), brentuximab (anti-CD30), Cantuzumab, Cibisatamab (anti-CEACAM5), Cirmtuzumab (anti-ROR1), cetuximab (anti-EGFR), Clivatuzumab (anti-MUC1), Gatipotuzumab (anti-MUC1), Coltuximab (anti-CD19), Daratumumab (anti-CD38), Duligotuzumab (anti-ERBB3), Edrecolomab (anti-EpCam), Enfortumab (anti-Nectin-4), Enoblituzumab (anti-CD276), Ensituximab (anti-MUC5AC), Epratuzumab (anti-CD22), Fartetuzumab (anti-FOLR1), Igovomab (anti-CA-125), Inebilizumab (anti-CD19), Iratumumab (anti-CD30), Labetuzumab (anti-CEA), Margetuximab (anti-HER2/neu), Matuzumab (anti-EGFR), Modotuximab (anti-EGFR extracellular domain III), Naptumomab (anti-5T4), Naratuximab (anti-CD37), Necitumumab (anti-EGFR), Nimotuzumab (anti-EGFR), Obinutuzumab (anti-CD20), Ocaratuzumab (anti-CD20), Ocrelizumab (anti-CD20), Ofatumumab (anti-CD20), Otlertuzumab (anti-CD37), Ontuxizumab (anti-TEM1), Pertuzumab (anti-HER2/neu), Polatuzumab (anti-CD79b), Rituximab (anti-CD20), Rovalpituzumab (anti-DLL3), Sacituzumab (anti-TROP-2), Seribantumab (anti-ERBB3/HER3), Tafasitamab (anti-CD19), Tetulomab (anti-CD37), Timigutuzumab (anti-HER2), Trastuzumab (anti-HER2), Ublituximab (anti-CD20), or Zolbetuximab (anti-Claudin 18.2).

26. The target-binding drug conjugate of claim 25, wherein the target-binding moiety has been genetically engineered to harbor a cysteine substitution in each of its heavy chains selected from A118C, S239C or D265C (according to EU numbering system), optionally D265C, wherein said target-binding moiety is conjugated to at least one modular linker compound according to any one of claims 1 to 8 via one or more of the engineered cysteine-residues of said target-binding moiety.

27. (canceled)

28. A pharmaceutical composition comprising the target-binding drug conjugate of claim 21.

29. (canceled)

30. A method of treating cancer, wherein the method comprises administering the target-binding drug conjugate of claim 21 to a subject in need thereof.
